(19) United States
(12) Patent Application Publication
(10) Pub. No.: US 2005/0255458 A1
(43) Pub. Date: Nov. 17, 2005

(54) Drug discovery assays based on the biology of chronic disease

(76) Inventor: Hanan Polansky, Rochester, NY (US)

Correspondence Address: Hanan Polansky, 3159 S. Winton Rd., Rochester, NY, 14623, US

(21) Appl. No.: 10/611,217

(22) Filed: Jul. 1, 2003

Related U.S. Application Data
(63) Continuation-in-part of application No. 10/223,050, filed on Aug. 14, 2002.

(51) Int. Cl. C12Q 1/70; C12Q 1/68

(52) U.S. Cl. 435/5; 435/6

(57) ABSTRACT

Using the recently discovered biology of chronic disease, the invention presents new methods for evaluating the effectiveness of a compound for use in modulating the progression of chronic disease, for determining whether a subject has a chronic disease, or has an increased risk of developing clinical symptoms associated with such disease, and for treating chronic disease.

### RELATED APPLICATIONS

This application is a Continuation-In-Part of application Ser. No. 10/223,050, entitled: Drug discovery assays based on microcompetition for a limiting GABP complex, filed on Aug. 14, 2002.

### BACKGROUND OF THE INVENTION

Today, the drug industry is in the midst of a crisis. The number of new drugs coming to the market is at its lowest point in a decade. According to William Haseltine¹, chief executive of U.S.-based Human Genome Sciences Inc., “Big pharmaceutical companies need to rethink their approach to drug discovery if the industry is to overcome a chronic decline in productivity . . . Many large firms are heading up a blind alley by focusing on conventional chemical-based medicines rather than biological products.” The reason for the decline is a limited understanding of the biology of chronic disease.

Biology and chemistry are interrelated disciplines. According to The American Heritage Dictionary, Fourth Edition², biology is “the science of life and of living organisms, including their structure, function, growth, origin, evolution, and distribution” while chemistry is “the science of the composition, structure, properties, and reactions of matter, especially of atomic and molecular systems.” Biologists describe life processes and chemists react to them. This is how treatments are developed. The biologist discovers the disruption (mutation, pathogen, toxin, etc.) that causes the disease, and the chemist finds the compounds that reverse the effect of the disruption.

The pharmaceutical industry recognizes this relationship. The following statements by Merck, Galxo-Wellcome, Novartis, and Vertex, three of the largest pharmaceutical companies in the world and a successful biotechnology company, illustrate the significance of biology in drug discovery: According to Merck³, “All vaccine and drug discovery projects begin with basic research. Whether the objective is to isolate, synthesize, or rationally design drug candidates, a thorough understanding of the targeted disease at a molecular level is essential.” According to Galxo-Wellcome⁴, “The more a gene product of interest is placed in a general biological context, the better the chance of finding a valid target.” According to Vertex⁵ (a biotech company), “We do not go into projects where the biology is uncertain . . . We look for the biology to be well understood. We look for the chemistry and the biophysics to be doable in a short period of time.”

Dr. Mark C. Fishman⁶, head of Novartis' new research center says, “To develop a drug that is both effective and well tolerated, you need to understand the molecular mechanism that causes a disease.” However, the molecular mechanism that causes chronic disease is unknown. Available treatments focus on clinical symptoms associated with the disease rather than the cause. As a result, in many cases, the treatments show limited efficacy and serious negative side effects.

The National Cancer Institute (NCI) at the NIH concurs, and as a result, recently announced a program (NIH Guide 2000⁷) aimed to “reorganize the “front-end,” or gateway, to drug discovery in cancer. The new approach promotes a three stage discovery process; first, discovery of the molecular mechanisms underlying neoplastic transformations, cancer growth and metastasis; second, selection of a novel molecular target within the discovered biochemical pathway associated with the disease state; finally, design of a new drug that modifies the selected target. The program encourages moving away from screening based on a clinical effects, such as tumor cell shrinkage, either in vivo or in vitro, to screening, or drug design, based on molecular effects. According to the NCI, screening by a desired clinical effect identified drugs that traditionally demonstrated clear limitations in patients, while screening by a desired molecular effect should produce more efficacious and specific drugs.

The best drugs reverse the molecular events that cause a disease. Following the discovery of microcompetition between foreign polynucleotides and cellular genes as the cause of many chronic diseases, the present invention presents methods for treating chronic diseases, methods for evaluating the effectiveness of a compound for use in modulating the progression of chronic diseases, and methods for determining whether a subject has a chronic disease, or has an increased risk of developing clinical symptoms associated with such disease.

### BRIEF SUMMARY OF THE INVENTION

In one aspect, the invention presents methods for treating chronic diseases. In a preferred embodiment, the methods feature administration to a subject a therapeutically effective amount of a pharmaceutical or nutraceutical composition that attenuates microcompetition between a foreign polynucleotide and a cellular polynucleotide, attenuates an effect of such microcompetition, or attenuates an effect of another foreign polynucleotide-type disruption. A pharmaceutical or nutraceutical composition may include, but not limited to, small molecule (organic or inorganic), polynucleotide, polypeptide, or antibody.

For example, to ameliorate a disease symptom resulting from microcompetition between a foreign polynucleotide and a cellular polynucleotide, a pharmaceutical composition can be administered to the subject that reduces the cellular copy number of the foreign polynucleotide, reduces complex formation between the foreign polynucleotide and a cellular transcription factor, increases complex formation between the microcompeted cellular transcription factor and the cellular polynucleotide, or reverses an effect of microcompetition on the expression or activity of a polypeptide with expression regulated by the cellular polynucleotide. For example, in the case of a p300/cbp virus and the cellular Rb gene, a pharmaceutical composition can be administered to the subject that reduces the copy number of the p300/cbp virus by, for instance, reducing viral replication, reduces binding of a p300/cbp transcription factor, such as GABP, to the p300/cbp virus, increases expression of the p300/cbp transcription factor, increases binding of the p300/cbp transcription factor to the Rb promoter by, for instance, stimulating phosphorylation of the p300/cbp transcription factor, or increases expression of Rb, through, for instance, transfection of an exogenous Rb gene, reduced degradation of the Rb protein, or administration of exogenous Rb protein (see more examples below).

In the case of another foreign polynucleotide-type disruption, for example, the composition may reverse the effects of such disruption. For instance, microcompetition with a p300/cbp virus reduces expression of Rb. A mutation can also reduce the expression of Rb. Therefore, such mutation is a foreign polynucleotide-type disruption. Microcompetition with a p300/cbp virus can result in cancer, and, therefore, a mutation in the Rb promoter that reduces Rb expression can also result in cancer. To ameliorate the symptoms of cancer resulting from such mutation in the Rb gene, a pharmaceutical composition can be administered to the subject that stimulates complex formation between a p300/cbp transcription factor and Rb.

In second aspect, the invention provides assays for screening test compounds to find compounds that modulate microcompetition between a foreign polynucleotide and a cellular polynucleotide, an effect of such microcompetition, or an effect of another foreign polynucleotide-type disruption. A further aspect of the invention provides methods for determining the risk of developing the molecular, cellular, and clinical symptoms associated with a chronic disease. The method may include detecting in a biological sample obtained from a subject at least one of the following: (i) a foreign polynucleotide, specifically, a p300/cbp virus (ii) modified expression or bioactivity of a gene susceptible to microcompetition with a foreign polynucleotide, specifically, a p300/cbp regulated gene (iii) presence of a genetic lesion in a gene susceptible to microcompetition with a foreign polynucleotide, specifically, a gene encoding a p300/cbp factor, a p300/cbp regulated gene, p300/cbp factor kinase or p300/cbp phosphatase, or p300/cbp agent (iv) presence of a genetic lesion in a DNA binding box of a p300/cbp transcription factor.

### BRIEF DESCRIPTION OF THE FIGURES

FIG. 1: A collection of observations as dots

FIG. 2: A theory

FIG. 3: Observed effect of pSV2neo on pSV2CAT expression.

FIG. 4: Observed effect of pSV-rMSV, pSV2Neo, and pA10 on pSV2CAT expression.

FIG. 5: Observed effect of pX1.0 on pSV2CAT expression.

FIG. 6: Observed effect of pSV2neo on pSV2CAT expression in Ltk- or ML fibroblast cells.

FIG. 7: Observed effect of pSV2neo on hMT-HA-CAT expression.

FIG. 8: Observed effect of pCMV-bgal, or pSV40-bgal on PDGF-B-CAT.

FIG. 9: Observed effect of increasing concentrations of the empty pSG5 vector on MMTV-luc expression cotransfected with pIRES-AR, pcDNA-AR or pSG5-AR, and treated with R1881, an AR ligand

FIG. 10: Observed effect of the empty pSG5 vector on bGAL and pSG5-luc expression in COS-7 cells cotransfected with pSG5-AR and treated with R1881.

FIG. 11: Graphical illustrations of adhesion functions calculated for “slower/lower,” “faster,” and “higher” increase in signal intensity.

FIG. 12: Graphical illustrations of velocity functions calculated for “slower/lower,” “faster,” and “higher” increase in signal intensity.

FIG. 13: Observed effect of integrin receptor expression on adhesion and velocity in a fibronectin “gradient.”

FIG. 14: Observed effect of integrin receptor affinity on adhesion and velocity in a fibronectin “gradient.”

FIG. 15: Calculated velocities and distribution of signal intensities corresponding to the [0,1] range of signal intensities.

FIG. 16: Predicted distribution of cell velocities corresponding to the [0,1] range of signal intensities sorted from low to high.

FIG. 17: Observed distribution of cell migration velocities.

FIG. 18: A velocity and corresponding probability.

FIG. 19: Observed effect of shear on the number of monocytes remaining bound.

FIG. 20: Effect of a decrease in “b” parameter on adhesion and velocity.

FIG. 21: Velocity distribution for different values of “b” parameter, assuming mean signal intensity=0.5, and SD of signal intensity=0.05.

FIG. 22: Velocity distribution for different values of “b” parameter, assuming mean signal intensity=0.5, and SD of signal intensity=0.1.

FIG. 23: Velocity distribution for different values of “b” parameter presented in histograms.

FIG. 24: Observed distribution of monocyte velocity on VCAM-1 following treatment with MCP-1.

FIG. 25: Observed distribution of monocyte velocity VCAM-1 in the presence of MCP-1 alone, or in combination with TS2/16.

FIG. 26: Adhesion and velocity as function of “b” parameter for three levels of signal intensity: 0.15, 0.30, and 0.45.

FIG. 27: Adhesion and velocity as a function of the “a” parameter for the three signal intensities: 0.15, 0.30, and 0.45.

FIG. 28: Adhesion as a function of time for “high” and “low” levels of “s” parameter.

FIG. 29: Adhesion and velocity as function of “s” parameter for the three signal intensities: 0.15, 0.30, and 0.45.

FIG. 30: Velocity as a function of skewness for a given signal intensity.

FIG. 31: Observed average monocyte velocity on VCAM-1 in the context of the skewed-bell model of cell motility.

FIG. 32: Migration distance as a function of time.

FIG. 33: Distance as function of “b” parameter for three time intervals [0,15], [0,30], and [0,45], where a=20 and s=4.

FIG. 34: Distance as function of “a” parameter for the three time intervals: [0,15], [0,30], and [0,45], where b=0.25 and s=4.

FIG. 35: Distance as function of “s” parameter for the three time intervals: [0,15], [0,30], and [0,45], where a=8.5 and b=0.5.

FIG. 36: Observed relations between PMN velocity and LTB4 concentrations, where x-axis is presented with a logarithmic scale, in control and psoriatic patients.

FIG. 37: Observed relations between PMN velocity and 12-HETE concentrations, where x-axis is presented with a logarithmic scale, in control and psoriatic patients.

FIG. 38: Observed relations between PMN velocity and 12-HETE concentrations, where x-axis is presented with a logarithmic scale, in mild and severe psoriatic patients.

FIG. 39: Observed relations between PMN velocity and LTB4 concentrations, where x-axis is presented with a linear scale, in control and psoriatic patients.

FIG. 40: Observed relations between PMN velocity and 12-HETE concentrations, where x-axis is presented with a linear scale, in control and psoriatic patients.

FIG. 41: Observed relations between PMN velocity and 12-HETE concentrations, where x-axis is presented with a linear scale, in mild and severe psoriatic patients FIG. 42: PDF of Burr distribution.

FIG. 43: PDF of Fisk distribution.

FIG. 44: PDF of extreme-value distribution with a lower bound (ExtremeLB).

FIG. 45: Dynamics of LDL pollution in the intima.

FIG. 46: Motility of an LDL trucking cell in the intima according to the skewed-bell model.

FIG. 47: Course reversal of trucking cells in the intima.

FIG. 48: Reverse transmigration over time.

FIG. 49: Fibrinogen in aorta of mice after a 2-month atherogenic diet.

FIG. 50: Fibrinogen in proximal aorta of apoE(−/−)Fibrinogen(+/−) mice.

FIG. 51: Observed fibronectin in endothelial layer (ECL), superficial area of the fatty streak plaque (INNER), and deep area of the fatty streak plaque (OUTER), in aorta of albino rabbits fed a high cholesterol-diet.

FIG. 52: Velocity curves for forward and backward, high and low skewness, “b” parameter case.

FIG. 53: Remoteness curves for low and high skewness, “b” parameter case.

FIG. 54: Velocity curves for forward and backward, high and low skewness, “a” parameter case.

FIG. 55: Remoteness curves for low and high skewness, “a” parameter case.

FIG. 56: Position of trucking cells in the intima assuming a uniform distribution over time of cell entry into the intima.

FIG. 57: Predicted effect of excessive skewness on distance traveled by SMC and Mf toward circulation.

FIG. 58: Predicted effect of a small decrease in skewness on distance traveled by SMC and Mf toward circulation.

FIG. 59: Predicted effect of a large decrease in skewness on distance traveled by SMC and Mf toward circulation.

FIG. 60: Exemplary grafts from control transplanted mice and apoAI transgenic transplanted mice stained with CD68, a macrophage specific marker (brown).

FIG. 61: Exemplary grafts from control transplanted mice and apoAI transgenic transplanted mice stained with a-actin, a smooth muscle cell specific marker (red).

FIG. 62: GSH content in U937 cells treated with 7-ketocholesterol.

FIG. 63: TF mRNA in human THP-1 cells following treatment with Cu+2 or LPS.

FIG. 64: Structure of plasminogen and its fragments.

FIG. 65: Structure of apo(a).

FIG. 66: Binding composition of fibronectin, plasminogen, and tissue factor.

FIG. 67: Predicted effect of foreign N-boxes and lipoprotein on number of trapped macrophages.

FIG. 68: Observed odds ratio of a MI event as a function of lipoprotein(a) concentration.

FIG. 69: Relation between lipoprotein(a), number of trapped macrophages, and centenarians.

FIG. 70: Observed serum lipoprotein(a) concentration in myocardial infarction and surgical operation patients over time

FIG. 71: Observed staining area for macrophages and SMC in aortic arch (A) and coronary artery (B) of control and apo(a) transgenic rabbits.

FIG. 72: Predicted effect of an: increase in tenascin-C on skewness and migration distance.

FIG. 73: Observed effect of tenascin-C on migration distance of U251.3 glioma cells over time.

FIG. 74: Observed dose effect of tenascin-C on migration distance of U251.3 glioma cells.

FIG. 75: Observed combined effect of tenascin-C and an antibody against a2 or b1 integrin on migration distance of U251.3 glioma cells.

FIG. 76: Observed migration distance of monocytes isolated from subjects at different age groups.

FIG. 77: Observed atherosclerotic lesions at different age groups.

FIG. 78: Observed dose effect of aspirin on leukocytes migration

FIG. 79: Observed effect of anti-CD40L on content of macrophages (A), lipid core (B), and SMC (C) in atherosclerotic lesions.

FIG. 80: Observed effect of anti-CD40L on content of macrophages (A), lipid (B), and SMC (C) in atherosclerotic lesions.

FIG. 81: Observed dose effect of angiotensin II on migration velocity of human neutrophils.

FIG. 82: Observed dose effect of angiotensin II on migration velocity of human and rat smooth muscle cells.

FIG. 83: Observed dose effects of angiotensin II on migration velocity of neutrophils and smooth muscle cells, overlaid.

FIG. 84: Angiotensin II inhibition as a decrease in skewness.

FIG. 85: Predicted effect of a change in angiotensin II gradient on skewness and migration distance.

FIG. 86: Statistical tests in Kowala 1995 (ibid).

FIG. 87: Cholesterol synthesis pathway.

FIG. 88: Predicted effect of statin on skewness and migration distance.

FIG. 89: Observed effect of pravastatin and simvastatin on macrophage, SMC, and lipid content in abdominal aortas of adult male cynomolgus monkeys fed an atherogenic diet.

FIG. 90: Observed effect of pravastatin and simvastatin on TF expression in abdominal aortas of adult male cynomolgus monkeys fed an atherogenic diet.

FIG. 91: Predicted effect of microcompetition with foreign N-boxes on skewness and migration distance of macrophages.

FIG. 92: A photomicrograph of atheroma (type IV lesion) in proximal left anterior descending coronary artery from a 23-year old man who died of a homicide. Extracellular lipids form a confluent core in the musculoelastic layer of eccentric adaptive thickening. The region between the core and the endothelial surface contains macrophages and foam cells (FC). “A” indicates adventitia, “M,” media. Fixation was performed by pressure-perfusion with glutaraldehyde, section thickness about 1-micron, magnification about ×55.

FIG. 93: A photomicrograph of thick part of atheroma (type IV lesion) in proximal left anterior descending coronary artery from a 19-year-old man who committed suicide. The core of extracellular lipids includes cholesterol crystals. Foam cells (FC) overlie the core. Macrophages, which are not foam cells (arrows), occupy the proteoglycan layer (pgc) adjacent to endothelium (E) at lesion surface. “A” indicates adventitia, “M,” media. Fixation was performed by pressure-perfusion with glutaraldehyde, section thickness about 1-micron, magnification about ×220.

FIG. 94: Predicted effect of microcompetition with foreign N-boxes on skewness and migration distance of SMC.

FIG. 95: Predicted effect of microcompetition with foreign N-boxes on skewness and migration distance of SMC in an area clear of LDL pollution.

FIG. 96: Some molecules on the surface of DCs and T-cells that participate in binding.

FIG. 97: Relation between [B7] or [Ag] and the probability of Th1 vs. Th2 differentiation.

FIG. 98: Relation between skewness, migration distance, and time.

FIG. 99: Copy of FIG. 34 in the technical note on cell motility.

FIG. 100: Two peak model of b cell apoptosis.

FIG. 101: Two peak dynamics.

FIG. 102: Autoimmune disease according to the two peak model.

FIG. 103: Observed b cell apoptosis in 5-6 week old male C57B1/6 mice following 5 daily injections of low dose streptozotocin.

FIG. 104: Observed islet area in 5-6 week old male C57B1/6 mice following 5 daily injections of low dose streptozotocin.

FIG. 105: Observed b cell apoptosis in 3-, and 12-week old male non-obese diabetic (NOD/Lt) mice following a single intraperitoneal injection of cyclophosphamide (CY), or in 12-week old mice following a single intraperitoneal injection of nicotinamide (NA) followed 15 minutes later by a single CY injection.

FIG. 106: Predicted effect of a transgenic increase in thioredoxin (TRX) expression in b cells on b cell apoptosis according to the two peak model.

FIG. 107: Predicted effect of CD40L, pulsing, apoptosis of tissue of bystander cells, and transfection with a gene expressing an epitope, on number of trapped DCs.

FIG. 108: Experimental configuration of Barratt-Boyes 2000 (ibid).

FIG. 109: Observed effect of an “empty” pZipNeo vector on hormone sensitive lipase (HSL) mRNA levels.

FIG. 110: Observed effect of five growth environments on the percentage of Rb-null (pRb(−/−)) 3T3 preadipocytes in S-phase.

FIG. 111: Predicted effect of microcompetition with foreign DNA on number of cell cycles required for production of a Rb concentration above growth arrest threshold.

FIG. 112: ERK/MAP kinase and JNK SAPK pathways.

FIG. 113: ERK phosphatases.

FIG. 114: Microcompetition with foreign DNA.

FIG. 115: Predicted effect of a control on expression of a GABP kinase agent.

FIG. 116: Sensitization and signal amplification.

FIG. 117: Sensitized AGENT receptor and hyper-AGENT-emia.

FIG. 118: Catecholamine, microcompetition with foreign DNA, and lipolysis.

FIG. 119: Observed effect of (A) norepinephrine, (B) isoprenaline, (C) forskolin, and (D) cdAMP on glycerol release in abdominal subcutaneous adipocytes from 13 non-obese subjects with at least one first-degree relative with a body mass index of 27 kg/m2 or more (Hob) and 14 controls (Hnorm).

FIG. 120: Observed effect of epinephrine infusion on glycerol release in obese and lean subjects.

FIG. 121: Observed relation between plasma epinephrine concentration and glycerol release in obese and lean subjects.

FIG. 122: Observed relation between plasma epinephrine concentration and total glycerol release per fat mass (FM) in obese and lean subjects.

FIG. 123: Experimental design of cited studies.

FIG. 124: Adipocyte differentiation in (A) non-transfected F442A cells (WT control), (B) cells transfected with pZIP-neo (“empty” vector control), and (C) cells transfected with the test gene plasmid.

FIG. 125: Observed growth of non-transfected cells, cells transfected with the pSV-Neo “empty” vector, and cells transfected with pBARB, the test gene plasmid. (A) Cells incubated in IS-RPMI with 5% FBS. (B) Cells incubated in serum free IS-RPMI.

FIG. 126: Observed growth of non-transfected cells, cells transfected with the pcDNA3 “empty” vector, and cells transfected with the antisense sequence, the test gene plasmid.

FIG. 127: Observed, growth of: O-tumors injected with the icon vector (test gene), D-tumors injected with pcDNA3.1(+) (“empty” vector control), s-uninjected tumors on the other flank in pcDNA3.1(+) injected mice (WT control).

FIG. 128: Observed growth of: O-tumors injected with the icon vector (test gene), D-tumors injected with pcDNA3.1 (+) (“empty” vector control), s-uninjected tumors on the other flank in pcDNA3.1(+) injected mice (WT control).

FIG. 129: Predicted effect of an infection with a GABP virus on the relation between signal intensity and adhesion (A) and between signal intensity and velocity (B).

FIG. 130: S-shaped curves representing fG-Complex1.

FIG. 131: Effect of the AR (−530, −140) segment on rate of transcription in HeLa and LNCaP cells.

FIG. 132: Effect of change in signal intensity on rate of transcription under “empty,” “full,” and “variable” boxes, and formation of stripes.

FIG. 133: Aggregate transcription rate, the “early ridge” shape.

FIG. 134: Aggregate transcription rate, the “late ridge” shape.

FIG. 135: Aggregate transcription rate, the “early gorge” shape.

FIG. 136: Observed effect of TPA on AR mRNA in Sertoli cells.

FIG. 137: Observed effect of FSH on AR mRNA in Sertoli cells

FIG. 138: Observed effect of TPA (A), ionomycin (B), and IL-6 (C) on 5a-RI mRNA levels in Jurkat cells.

FIG. 139: Observed effect of cycloheximide on AR mRNA levels in Sertoli cells in context of the SS model of transcription.

FIG. 140: Observed effect of ATRA on TF mRNA levels in THP-1 cells in context of the SS model of transcription and.

FIG. 141: Predicted effect of R1881 on AR rate pf transcription according to the SS model of transcription.

FIG. 142: Observed effect of R1881 on LUC activity in LNCaP cells transfected with the pSLA3-H2/3-E3k vector that expresses LUC under control of the (−1400, +966) segment of the AR promoter.

FIG. 143: Observed effect of R1881 on LUC activity in LNCaP cells transfected with pSLA3-GRE-Oct, a vector that expresses LUC under control of a promoter that includes a glucocorticoid response element (GRE) in front of the minimal Oct-6 promoter, or pSLA3-H2/3-E3k, a vector that expresses LUC under control of the (−1400, +966) segment of the AR promoter.

FIG. 144: SS model of transcription for a signal that is an exclusive suppresser.

FIG. 145: Predicted effect of ATRA on LUC activity in cells transfected with a vector that expresses LUC under control of the TF promoter according to the SS model of transcription.

FIG. 146: Structure of a hair follicle.

FIG. 147: Synthesis of DHT.

FIG. 148: Observed growth rate of the dermal papilla cells isolated from anagen hair follicles of prepubertal juvenile prebald frontal scalp (“juvenile prebald frontal DP”), adult bald frontal scalp (“adult bald frontal DP”), and adult occipital scalp (“adult occipital DP”) of stumptailed macaques.

FIG. 149: Growth rate of dermal papilla cells isolated from non-balding and balding areas in culture supplemented with 20% human serum (HS): (A) or 20% fetal calf serum (FCS) (B).

FIG. 150: Observed effect of topical treatment with testosterone, DHT, 17b-estradiol, or acetone vehicle alone, on percent of mice with hair regrowth.

FIG. 151: Experimental configuration in Hodgins 1991 (ibid) and Hibberts 1998 (ibid).

FIG. 152: Pathways of arachidonic acid (AA) oxidation.

FIG. 153: Potential targets of treatment agents.

FIG. 154: Distances between equilibrium points.

FIG. 155: Observed effect of sodium butyrate on metallothionein (MT) mRNA levels.

FIG. 156: Observed effect of acarbose on body weight of non-insulin-dependent diabetes (NIDDM) patients.

FIG. 157: Observed effect of sodium orthovanadate on mRNA levels of the F-type PFK-2/FBPase-2 gene in rats with streptozotocin (STZ)-induced diabetes.

FIG. 158: Observed effect of AZT monotherapy, a combination of AZT+ddC, or a combination of AZT+ddI therapy on HIV-1 DNA and RNA load in 42 antiretroviral naive HIV-1 infected persons.

FIG. 159: Regression analysis with viral DNA level as dependent variable and number of years since seroconversion as independent variable.

### DETAILED DESCRIPTION OF THE INVENTION

A. Introduction of Invention

1. Detailed Description of New Elements

The following sections present descriptions of elements used in the present invention. Following each definition, one or more exemplary assays are provided to illustrate to one skilled in the art how to use the element. Each assay may include, as its own elements, standard methods in molecular biology, microbiology, cell biology, cell culture, transgenic biology, recombinant DNA, immunology, pharmacology, and toxicology, well known in the art. Details of the standard methods are available further below.

a) Microcompetition Related Elements

(1) Microcompetition

Definition

Assume the DNA sequences DNA₁ and DNA₂ bind the transcription complexes C₁ and C₂, respectively. If C₁ and C₂ include the same transcription factor, DNA₁ and DNA₂ are called “microcompetitors.” A special case of microcompetition is two DNA sequences that bind the same transcription complex.

Notes:

1. Transcription factors include transcription coactivators.

2. Sharing the same environment, such as cell, or chemical mix, is not required, to be regarded microcompetitors. For instance, two genes, which were shown once to bind the same transcription factor are, regarded microcompetitors independent of their actual physical environment. To emphasize such independence, the terminology “susceptible to microcompetition” may be used.

Exemplary Assays

1. If DNA₁ and DNA₂ are endogenous in the cell of interest, assay the transcription factors bound to the DNA sequences (see in “Detailed description of standard protocols” below, the section entitled “Identifying azpolypeptide bound to DNA or protein complex”) and compare the two sets of polypeptides. If the two sets include a common transcription factor, DNA₁ and DNA₂ are microcompetitors.

2. In the previous assay, if DNA₁ and/or DNA₂ are not endogenous, introduce DNA₁ and/or DNA₂ to the cell by, for instance, transfecting the cell with plasmids carrying DNA, and/or DNA₂, infecting the cell with a virus that includes DNA₁ and/or DNA₂, and mutating endogenous DNA to produce a sequence identical to DNA₁ and/or DNA₂.

Notes:

1. Introduction of exogenous DNA₁ and/or DNA₂ is a special case of modifying the cellular copy number of a DNA sequence. Such introduction increases the copy number from zero to a positive number. Generally, copy number may be modified by means such as the ones mentioned above, for instance, transfecting the cell with plasmids carrying a DNA sequence of interest, infecting the cell with a virus that includes the DNA sequence of interest, and mutating endogenous DNA to produce a sequence identical to the DNA sequence of interest.

2. Assume DNA₁ and DNA₂ microcompete for the transcription factor F. Assaying the copy number of at least one of the two sequences, that is, DNA₁ and/or DNA₂, is regarded as assaying microcompetition for F, and observing a change in the copy number of at least one of the two sequences is regarded as identification of modified microcompetition for F.

3. Assume the transcription factor F binds the DNA box DNA_{F}. Consider a specific DNA sequence, DNA₁ that includes a DNA_{F} box, then:
[F·DNA₁]=f([DNA_{F}], [F], F-affinity, F-avidity) Function 1

The concentration of F bound to DNA₁ is a function of the DNA_{F} copy number, the concentration of F in the cell, F affinity and avidity to its box. Using function f, a change in microcompetition can be defined asia change in [DNA_{F}], and a change in [F·DNA₁] as an effect of such change.

4. Note that under certain conditions (fixed [F], fixed F-affinity, fixed F-avidity, and limiting transcription factor (see below)), there is a “one to one” relation between [F·DNA₁] and [DNA_{F}]. Under such conditions, assaying [F·DNA₁] is regarded assaying microcompetition.

Examples

See studies in the section below entitled “Microcompetition with a limiting transcription complex.”

(2) Microavailable

Definition

Let L₁ and L₂ be two molecules. Assume L₁ can take s=(1 . . . n) shapes. Let L_{1,s} denote L₁ in shape s, and let [L_{1,s}] denote concentration of L_{1,s}. If L_{1,s} can bind L₂, an increase (or decrease) in [L_{1,s}] in the environment of L₂ is called “increase (or decrease) in microavailability of L_{1,s} to L₂.” Microavailability of L_{1,s} is denoted ₘₐL_{1,s}. A shape that does not bind L₂ is called “microunavailable to L₂.”

Let s=(1 . . . m) denote the set of all L_{1,s} that can bind L₂. Any increase (or decrease) in the sum of [L_{1,s}] over all s=(1 . . . m) is called “increase (or decrease) in microavailability of L₁ to L₂.” Microavailability of L₁ to L₂ is denoted ₘₐL₁.

Notes:

1. A molecule in a complex is regarded in a different shape relative to the same molecule uncomplexed, or free.

2. Consider, for example, an antibody against L_{1,J}, a specific shape of L₁. Assume the antibody binds L_{1,j} in the region contacting L₂. Assume the antibody binds a single region of L_{1,j}, and that antibody binding prevents formation of the L₁·L₂ complex. By binding L_{1,j}, the antibody changes the shape of L₁ from L_{1,j} to L_{1,k} (from exposed to hidden contact region). Since L_{1,k} does not bind L₂, the decrease in [L_{1,j}] decreases ₘₐL₁, or the microavailability of L₁ to L₂. If, on the other hand, the antibody converts L_{1,j} to L_{1,p}, a shape that also forms the L₁·L₂ complex with the same probability, ₘₐL₁ is fixed. The decrease in [L_{1,j}] is equal to the increase in [L_{1,p}], resulting in a fixed sum of [L_{1,s}] computed over all s that bind L₂.

Exemplary Assays

The following assays identify a change in ₘₐL₁ following treatment.

1. Assay in a biological system (e.g., cell, cell lysate, chemical mixture) the concentrations of all L_{1,s} where s is a shape that can bind L₂. Apply a treatment to the system which may change L_{1,s}. Following treatment, assay again the concentrations of all L_{1,s}, where s is a shape that can bind L₂. Calculate the sum of [L_{1,s}] over all s, before and after treatment. An increase (or decrease) in this sum indicates an increase (or decrease) in ₘₐL₁.

Examples

Antibodies specific for L_{1,s} may be used in immunoprecipitation, Western blot or immunoaffinity to quantify the levels of L_{1,s} before and after treatment. See also examples below.

(3) Limiting Transcription Factor

Definition

Assume the transcription factor F binds DNA₁. F is called “limiting with respect to DNA₁,” if a decrease in microavailability of F to DNA, decreases the concentration of F bound to DNA₁ (“bound F”).

Notes:

1. The definition characterizes “limiting” by the relation between the concentration of microavailable F and the concentration of F actually bound to DNA₁. According to the definition, “limiting” means a direct relation between a decrease in microavailable F and a decrease in bound F, and “not limiting” means no such relation between the two variables. For instance, according to this definition, a decrease in microavailable F with no corresponding change in bound F, means, “not limiting.”

2. Let G₁ denote a DNA sequence of a certain gene. Such DNA sequence may include coding and non-coding regions of a gene, such as exons, introns, promoters, enhancers, or other segments positioned 5′ or 3′ to the coding region. Assume the transcription factor F binds G₁. An assay can measure changes in G₁ mRNA expression instead of changes in the concentration of bound F. Assume F transactivates G₁. Since F is necessary for transcription, a decrease in ₘₐF decreases F·G₁, which, in turn, decreases G₁ transcription. However, an increase in concentration of F bound to G₁ does not necessarily increase transcription if binding of F is necessary but not sufficient for transactivation of G₁.

Exemplary Assays

1. Identify a treatment that decreases ₘₐF by trying different treatments, assaying ₘₐF following each treatment, and choosing a treatment that decreases ₘₐF. Assay concentration of F bound to DNA₁ in a biological system (e.g. cell). Use the identified treatment to decrease ₘₐF. Following treatment, assay again the concentration of bound F. A decrease in the concentration of F bound to DNA₁ indicates that F is limiting with respect to DNA₁.

2. Transfect a recombinant expression vector carrying the gene expressing F. Expression of this exogenous F will increase the intracellular concentration of F. Following transfection:
(a) Assay the concentration of F bound to DNA₁. An increase in concentration of bound F indicates that F is limiting with respect to DNA₁.
(b) If DNA, is the gene G₁, assay G₁ transcription. An increase in G₁ transcription indicates that F is limiting with respect to G₁ (such an increase in transcription is expected if binding of F to G₁ is sufficient for transactivation).

3. Contact a cell with antibodies that decrease ₘₐF. Following treatment:
(a) Assay the concentration of F bound to DNA₁. A decrease in concentration of bound F with any antibody concentration indicates that F is limiting with respect to DNA₁.
(b) If DNA, is the gene G₁, assay G₁ transcription. A decrease in G₁ transcription with any antibody concentration indicates that F is limiting with respect to G₁.

See Kamei 1996 (ibid) that used anti-CBP immunoglubulin G (IgG). (Instead of antibodies, some studies used E1A, which, by binding to p300, also converts the shape from microavailable to microunavailable.)

4. Modify the copy number of DNA₂, another DNA sequence, or G₂, another gene, which also bind F (by, for instance, transfecting the cell with DNA₂ or G₂, see above).
(a) Assay the concentration of F bound to DNA₁. A decrease in concentration of F bound to DNA₁ indicates that F is limiting with respect to DNA₁.
(b) If DNA₁ is the gene G₁, assay G₁ transcription. A decrease in G₁ transcription indicates that F is limiting with respect to G₁.

If DNA₁ is the gene G₁, competition with DNA₂ or G₂, which also bind F, decreases the concentration of F bound to G₁ and, therefore, the resulting transactivation of G₁ in any concentration of DNA₂ or G₂. In respect to G₁, binding of F to DNA₂ or G₂ decreases microavailability of F to G₁, since F bound to DNA₂ or G₂ is microunavailable for binding with G₁.

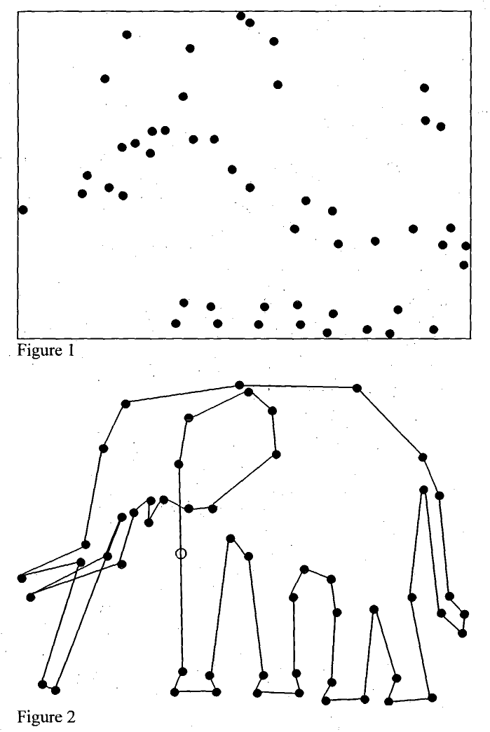

This assay is exemplified in a study reported by Kamei 1996 (ibid). The study used TPA to stimulate transcription from a promoter containing an AP-1 site. AP-1 interacts with CBP. CBP also interacts with a liganded retinoic acid receptor (RAR) and liganded glucocorticoid receptor (GR) (Kamei 1996, ibid, FIG. 1). Both RAR and GR exhibited ligand-dependent repression of TPA stimulated transcription. Induction by TPA was about 80% repressed by treatment with retinoic acid or dexamethasone. In this study, G is the gene controlled by the AP-1 promoter. In respect to this gene, the CBP·liganded-RAR complex is the microunavailable form. An increase in [CBP·liganded-RAR] decreases the concentration of microavailable CBP.

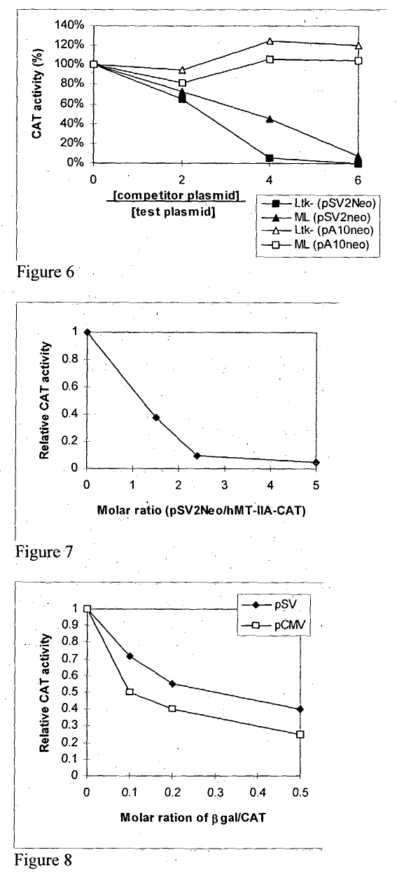

In another study (Hottiger 1998, ibid), the two genes are HIV-CAT, which binds NF-κB, and GAL4-CAT, which binds the fusion protein GAL4-Stat2(TA). NF-κB binds p300. The GAL4-Stat2(TA) fusion protein includes the Stat2 transactivation domain that also binds p300. The study showed a close dependent inhibition of gene activation by the transactivation domain of Stat2 following transfection of a RelA expression vector (Hottiger 1998, ibid, FIG. 6A).

5. Transfect F and modify the copy number of DNA₂, another DNA sequence, or G₂, another gene, which also bind F (by, for instance, transfecting the cell with DNA₂ or G₂, see also above).

Following Transfection:

(a) Assay concentration of F bound to DNA₁. Attenuated decrease in concentration of F bound to DNA₁ indicates that F is limiting with respect to DNA₁.
(b) If DNA1 is the gene G₁, assay G₁ transcription. Attenuated decrease in G₁ transactivation caused by DNA₂ or G₂ indicates that F is limiting with respect to G₁ (see Hottiger 1998, ibid, FIG. 6D).

6. Call the box that binds F the “F-box.” Transfect a cell with DNA₂, another DNA sequence, or G₂, another gene carrying a wild type F-box. Transfect another cell with DNA₂ or G₂, after mutating the F-box in the transfected DNA₂ or G₂.
(a) Assay the concentration of F bound to DNA₁. Attenuated decrease in the concentration of F bound to DNA₁ with the wild type but not the mutated F-box indicates that F is limiting with respect to DNA₁.
(b) If DNA1 is the gene G₁, assay G₁ transcription. Attenuated decrease in G₁ transactivation with the wild type but not the mutated F-box indicates that F is limiting with respect to G₁.

If DNA1 is the gene G₁, a mutation in the F-box results in diminished binding of F to DNA₂ or G₂, and an attenuated inhibitory effect on G₁ transactivation. In Kamei 1996 (ibid), mutations in the RAR AF2 domain that inhibit binding of CBP, and other coactivator proteins, abolished AP-1 repression by nuclear receptors.

7. Let t₁ and t₂ be two transcription factors that bind F. Let G₁ and G₂ be two genes transactivated by the t₁·F and t₂·F complexes, respectively.
(a) Transfect a cell of interest with t₁ and assay G₂ transcription. If the increase in [t₁] decreases transcription of G₂, F is limiting with respect to G. Call t₂·F the microavailable shape of F with respect to G₂. The increase in [t₁] increases [t₁·F], which, in turn, decreases [t₂·F]. The decrease in the shape of F microavailable to G₂ decreases transactivation of G₂. In Hottiger 1998 (ibid), t₁ is RelA, t₂ is GAL4-Stat2(TA) and G₂ is GAL4-CAT. See the effect of the increase in t₁ on G₂ transactivation in Hottiger (1998, ibid) FIG. 6A.
(b) Transfect F and assay the concentration of F bound to G, or transactivation of G. If the increase in F decreases the inhibitory effect of t₁, F is limiting with respect to G (see Hottiger 1998 (ibid), FIG. 6C showing the effect of p300 transfection).
(c) Assay the concentration of t₁, t₂, and F. If t₁ and t₂ have high molar excess compared to F, F is limiting with respect to G (see Hottiger 1998, (ibid)).

(4) Microcompetition for a Limiting Factor

Definition

Assume DNA₁ and DNA₂ microcompete for the transcription factor F. If F is limiting with respect to DNA₁ and DNA₂, DNA₁ and DNA₂ are called “microcompetitors for a limiting factor.”

Exemplary Assays

1. The assays 4-7 in the section entitled “Limiting transcription factor” above (p 16), can be used to identify microcompetition for a limiting factor.

2. Modify the copy number of DNA₁ and DNA₂ (by, for instance, co-transfecting recombinant vector carrying DNA₁ and DNA₂, see also above).
(a) Assay DNA₁ protection against enzymatic digestion (“DNase footprint assay”). A change in protection indicates microcompetition for a limiting factor.
(b) Assay DNA₁ electrophoretic gel mobility (“electrophoretic mobility shift assay”). A change in mobility indicates microcompetition for a limiting factor.

3. If DNA₁ is a segment of a promoter or enhancer, or can function as a promoter or enhancer, independently, or in combination of other DNA sequences, fuse DNA₁ to a reporter gene such as CAT or LUC. Co-transfect the fused DNA₁ and DNA₂. Assay for expression of the reporter gene. Specifically, assay transactivation of reporter gene following an increase in DNA₂ copy number. A change in transactivation of the reporter gene indicates microcompetition for a limiting factor.

4. A special case is when DNA₁ is the entire cellular genome responsible for normal cell morphology and function. Transfect DNA₂, and assay cell morphology and/or function (such as, binding of extracellular protein, cell replication, cellular oxidative stress, gene transcription, etc.). A change in cell morphology and/or function indicates microcompetition for a limiting factor.

Note: Preferably, following co-transfection of DNA₁ and DNA₂, verify that the polynucleotides do not produce mRNA. If the sequences transcribe mRNA, block translation of proteins with, for instance, an antisense oligonucleotide specific for the exogenous mRNA. Alternatively, verify that the proteins are not involved in binding of F to either sequence. Also, verify that co-transfection does not mutate the F-boxes in DNA₁ and DNA₂, and that the sequences do not change the methylation patterns of their F-boxes. Finally, check that DNA₁ and DNA₂ do not contact each other in the F-box region.

Examples

See studies in the section below entitled “Microcompetition, with a limiting transcription complex.”

(5) Foreign to

Definition 1

Consider an organism R with standard genome O. Consider Oₛ a segment of O. If a polynucleotide Pn is different from Oₛ for all Oₛ in O, Pn is called “foreign to R.”

Notes:

1. As example for different organisms, consider the list of standard organisms in the PatentIn 3.1 software. The list includes organisms such as, homo sapiens (human), mus musculus (mouse), ovis aries (sheep), and gallus gallus (chicken).

2. A standard genome is the genome shared by most representatives of the same organism.

3. A polynucleotide and DNA sequence (see above) are interchangeable concepts.

4. In multicellular organism, such as humans, the standard genome of the organism is not necessarily found in every cell. The genomes found in sampled cells can vary as a result of somatic mutations, viral integration, etc. (see definition below of foreign polynucleotide in a specific cell).

5. Assume Pn expresses the polypeptide Pp. If Pn is foreign to R, then Pp is foreign to R.

6. When the reference organism is evident, instead of the phrase “a polynucleotide foreign to organism R,” the “foreign polynucleotide” phrase might be used.

Exemplary Assays

1. Compare the sequence of Pn with the sequence, or sequences of the published, or self sequenced standard genome of R. If the sequence is not a segment of the standard genome, Pn is foreign to R.

2. Isolate DNA from O (for instance, from a specific cell, or a virus). Try to hybridize Pn to the isolated DNA. If Pn does not hybridize, it is foreign.

Notes:

1. Pn can still be foreign if it hybridizes with DNA from a specific O specimen. Consider, for example, the case of integrated viral genomes. Viral sequences integrated into cellular genomes are foreign. To increase the probability of correct identification, repeat the assay with N>1 specimens of O (for instance, by collecting N cells from different representatives of R). Define the genome of R as all DNA sequences found in all O specimens. Following this definition, integrated sequences, which are only segments of certain O specimens, are identified as foreign. Note that the test is dependent on the N population. For instance, a colony, which propagates from a single cell, might include a foreign polynucleotide in all daughter cells. Therefore, the N specimens should include genomes (or cells) from different lineages.

2. A polynucleotide can also be identified as potentially foreign if it is found episomally in the nucleus. If the DNA is found in the cytoplasm, it is most likely foreign. In addition, a large enough polynucleotide can be identified as foreign if many copies of the polynucleotide can be observed in the nucleus. Finally, if Pn is identical to sequences in genomes of other organisms, such as viruses or bacteria, known to invade R cells, and specifically nuclei of R cells, Pn is likely foreign to R.

Definition 2

Consider an organism R. If a polynucleotide Pn is immunologically foreign to R, Pn is called “foreign to R.”

Notes:

1. In Definition 1, the comparison between O, the genome of R, and Pn is performed logically by the observer. In definition 2, the comparison is performed biologically by the immune system of the organism R.

2. Definition 2 can be generalized to any compound or substance. A compound X is called foreign to organism R, if X is immunologically foreign to R.

Exemplary Assays

1. If the test polynucleotide includes a coding region, incorporate the test polynucleotide in an expressing plasmid and transfer the plasmid into organism R, through, for instance, injection (see DNA-based immunization protocols). An immune response against the expressed polypeptide indicates that the polynucleotide is foreign.

2. Inject the test polynucleotide in R. An immune response against the injected polynucleotide indicates that the test polynucleotide is foreign.

Examples

Many nuclear viruses, such as Epstein-Barr, and cytoplasmic viruses, such as Vaccinia, express proteins that are antigenic and immunogenic in their respective host cells.

Definition 3

Consider an organism R with standard genome O. Consider Oₛ, a segment of O. If a polynucleotide Pn is chemically or physically different than Oₛ for all Oₛ in O, Pn is called “foreign to R.”

Note: In Definition 3, the observer compares O, the genome of the R organism, with Pn using the molecules chemical or physical characteristics.

Exemplary Assays

In general, many assays in the “Detection of a genetic lesion” section below compare a test polynucleotide and a wild-type polynucleotide. In these assay, let OS be the wild-type polynucleotide and use the assays to identify a foreign polynucleotide. Consider the following examples.

1. Compare the electrophoretic gel mobility of Oₛ and the test polynucleotide. If mobility is different, the polynucleotides are different.

2. Compare the patterns of restriction enzyme cleavage of Oₛ and the test polynucleotide. If the patterns are different, the polynucleotides are different.

3. Compare the patterns of methylation of Oₛ and the test polynucleotide (by, for instance, electrophoretic gel mobility). If the patterns are different, the polynucleotides are different.

Definition 4

Consider an organism R with standard genome O. Let [Pn] denote the copy number of Pn in O. Consider a cell Cellᵢ. Let [Pn]ᵢ denote the copy number of Pn in Cellᵢ. If [Pn]ᵢ>[Pn], Pn is called “foreign to Cellᵢ.”

Notes:

1. [Pn]ᵢ is the copy number of all Pn in Cellᵢ, from all sources. For instance, [Pn] includes all Pn segments in O, all Pn segments of viral DNA in the cell (if available), all Pn segments of plasmid DNA in the cell (if available), etc.

2. If [Pn]=0, the definition is identical to definition 1 of foreign polynucleotide.

Exemplary Assays

1. Sequence the genome of Cellᵢ. Count the number of time Pn appears in the genome. Compare the result to the number of times Pn appears in the published standard genome. If the number is greater, Pn is foreign to Cellᵢ.

2. Sequence the genome of Cellᵢ and a group of other cells Cellⱼ, . . . , Cellⱼ₊ₘ. If [Pn]ᵢ>[Pn]ⱼ= . . . =[Pn]ⱼ₊ₘ, Pn is foreign to Cellᵢ.

(6) Natural to

Definition

Consider an organism R with standard genome O. If a polynucleotide Pn is a fragment of O, Pn is called “natural to R.”

Notes:

1. “Natural to” and “foreign to” are mutually exclusive. A polynucleotide cannot be both foreign and natural to R. If a polynucleotide is natural, it is not foreign to R, and if a polynucleotide is foreign, it is not natural to R.

2. If Pn is a gene natural to R, then, its gene product is also natural to R.

3. The products of a reaction carried out in a cell between gene products natural to the cell, under normal conditions, are natural to the cell. For instance, cellular splicing by factors natural to the cell produce splice products natural to the cell.

Exemplary Assays,

1. Compare the sequence of Pn with the sequence, or sequences of the published, or self sequenced standard genome of R. If the sequence is a segment of the standard genome, Pn is natural to R.

2. Isolate DNA from O (for instance, from a specific cell, or a virus). Try to hybridize Pn to the isolated DNA. If Pn hybridizes, it is natural.

Note: Hybridization with DNA from a specific O specimen of R is not conclusive evidence that Pn is natural to R. Consider, for example, the case of integrated viral genomes. Viral sequences integrated into cellular genomes are foreign. To increase the probability of correct identification, repeat the assay with N>1 specimens of O (for instance, by collecting N cells from different representatives of R). Define the genome of R as all DNA sequences found in all O specimens. Following this definition, integrated sequences, which are only segments of certain O specimens, are identified as foreign. Note that the test is dependent on the N population. For instance, a colony, which propagates from a single cell, right include a foreign polynucleotide in all daughter cells. Therefore, the N specimens should include genomes (or cells) from different lineages.

(7) Empty Polynucleotide

Definition

Consider the Pn polynucleotide. Consider an organism R with genome O_{R}. Let Pp(Pn), and Pp(O_{R}) denote a gene product (polypeptide) of a Pn or O_{R} gene, respectively. If Pp(Pn)≠Pp(O_{R}) for all Pp(Pn), Pn will be called an “empty polynucleotide” with respect to R.

Notes:

1. A vector is a specific example of a polynucleotide.

2. A vector that includes a non coding polynucleotide natural to R is considered empty with respect to R. (“natural to” is the opposite of “foreign to.” Note: A natural polynucleotide means, a polynucleotide natural to at least one organism. An artificial polynucleotide means a polynucleotide foreign to all known organisms. A viral enhancer is a natural polynucleotide. A plasmid with a viral enhancer fused to a human gene is artificial.)

3. A vector that includes a coding gene natural to Q, an organism different from R, can still be considered empty with respect to R. For instance, a vector that includes the bacterial chloramphenicol transacetylase (CAT), bacterial neomycin phosphotransferase (neo), or the firefly luciferase (LUC) as reporter genes, but no human coding gene is considered empty with respect to humans if it does not express a gene natural to humans.

Exemplary Assays

1. Identify all gene products encoded by Pn. Compare to the gene products of O_{R}. If all gene products are different, Pn is considered empty with respect to R.

Examples

pSV2CAT, which expresses the chloramphenicol acethyltransferase (CAT) gene under the control of the SV40 promoter/enhancer, pSV2neo, which expresses the neo gene under the control of the SV40 promoter/enhancer, HSV-neo, which expresses the neomycin-resistance gene under control of the murine Harvey sarcoma virus long terminal repeat (LTR), pZIP-Neo, which expresses the neomycin-resistant gene under control of the Moloney murine leukemia virus long terminal repeat (LTR), are considered empty polynucleotides, or empty vectors, with respect to humans and to the respective virus. See more examples below.

Note: These vectors can be considered as “double” empty, empty with respect to humans, and empty with respect to the respective virus.

(8) Latent Foreign Polynucleotide

Definition

Consider Pn, a polynucleotide foreign to organism R. Pn will be called latent in a Cellᵢ of R if over an extended period of time, either:

1. Pn produces no Pn transcripts.

2. Denote the set of gene products expressed by Pn in Cellᵢ with Cellᵢ Pp(Pn) and the set of all possible gene products of Pn with All_Pp(Pn), then, Cell_{i—}Pp(Pn)⊂All_Pp(Pn), that is, the set of Pn gene products expressed in Cellᵢ is a subset of all possible Pn gene products.

3. Pn shows limited or no replication.

4. Pn is undetected by the host immune system.

5. Cellᵢ shows no lytic symptoms.

6. R shows no macroscopic symptoms.

Notes:

1. A virus in a host cell is a foreign polynucleotide. According to the definition, a virus is considered latent if, over an extended period of time, it either shows partial expression of its gene products, no viral mRNA, limited or no replication, is undetected by the host immune system, causes no lytic symptoms in the infected cell, or causes no macroscopic symptoms in the host.

2. The above list of characterizations is not exhaustive. The medical literature includes more aspects of latency that can be added to the definition.

3. Some studies use the terms persistent infection or abortive replication instead of latent infection.

Exemplary Assays

1. Introduce, or identify a foreign polynucleotide in a host cell. Assay the polynucleotide replication, or transcription, or mRNA, or gene products over an extended period of time. If the polynucleotide shows limited replication, no transcription, or a limited set of transcripts, the polynucleotide is latent.

2. Introduce, or identify a foreign polynucleotide in a host cell. Assay the cell over an extended period of time, if the cell shows no lytic symptoms, the polynucleotide is latent.

Examples

Using PCR, a study (Gonelli 2001⁹) observed persistent presence of viral human herpes virus 7 (HHV-7) DNA in biopsies from 50 patients with chronic gastritis. The study also observed no U14, U17/17, U31, U42 and U89/90, HHV-7 specific transcripts highly expressed during replication. Based on these observations, the study concluded: “gastric tissue represents a site of HHV-7 latent infection and potential reservoir for viral reactivation.” To test the effect of treatment on the establishment of latent herpes simplex virus, type 1 (HSV-1) in sensory neurons, another study (Smith 2001¹⁰) assays the expression of the latency-associated transcript (LAT), the only region of the viral genome transcribed at high levels during the period of viral latency. A recent review (Young 2000¹¹) discusses the limited sets of Epstein-Barr viral (EBV) gene products expressed during the period of viral latency.

(9) Partial Description

Definition

Let cᵢ be a characteristic of a system. For every cᵢ, assume a non-trivial range of values. Let the set C={cᵢ|1≦i≦m} be the set of characteristics providing a complete description of the system. Any subset of C will be called a “partial description” of the system.

Exemplary Assays

1. Chose any set of characteristics describing the system and assay these characteristics.

Examples

Assaying blood pressure, blood triglycerides, glucose tolerance, body weight, etc. produces a partial description of a system.

(10) Equilibrium

Definition

The set of C characteristics where every characteristic is represented by one value from its respective range of values will be called a state, denoted St(C).

Definition

If a system persists in a state St(C)=St₀ over time, St₀ is called equilibrium.

Note: The definitions can be modified to accommodate partial descriptions. For example, consider a description of a system that includes the set Cₖ, which is a proper subset of C (Cₖ⊂C). Consider a state St(Cₖ)=St₁. If the system persists over time in St₁, the probability that the system is in equilibrium is greater than zero However, since the system is categorizes based on a subset of C, the probability is less than 1. Overall, an increase in the size of the subset of characteristics increases the probability.

Exemplary Assays

1. Assay the values of the complete (sub) set of the system characteristics. Repeat the assays over time. If the values persist, the system is (probably) in equilibrium.

Examples

Regular physicals include standard tests, such as blood count, cholesterol levels, HDL, cholesterol, triglycerides, kidney function tests, thyroid function tests, liver function tests, minerals, blood sugar, uric acid, electrolytes, resting electrocardiogram, an exercise treadmill test, vision testing, and audiometry. When the values in these tests remain within a narrow range over time, the medical condition of the subject can be labeled as a probable equilibrium. Other tests performed to identify deviations from equilibrium are mammograms and prostate cancer screenings.

(11) Stable Equilibrium

Definition

Consider equilibrium E₀. If, after small disturbances, the system always returns to E₀, the equilibrium is called “stable.” If the system moves away from E₀ after small disturbances, the equilibrium is called “unstable.”

Exemplary Assays

1. Take a biological system (e.g., cell, whole organism, etc.). Assay a set of characteristics. Verify that the system is in equilibrium, that is, the values of these characteristics persist over time. Apply treatment to the system and assay the set of characteristics again. Repeat assaying over time. If the treatment changed the values of the characteristics, and within a reasonable time the values returned to the original levels, the equilibrium is stable.

(12) Chronic Disease

Definition

Let a healthy biological system be identified with a certain stable equilibrium. A stable equilibrium different from the healthy system equilibrium is called “chronic disease.”

Note: In chronic disease, in contrast to acute disease, the system does not return to the healthy equilibrium on its own.

Exemplary Assays

1. Take a biological system (e.g., cell, whole organism, etc.). Assay a set of characteristics. Compare the results with the values of the same characteristics in healthy controls. If some values deviate from the values of healthy controls, and the values continue to deviate over time, the equilibrium of the system can be characterizes as chronic disease.

Examples

High blood pressure, high body weight, hyperglycemia, etc. indicate a chronic disease.

(13) Disruption

Definition

Let a healthy biological system be identified with a certain stable equilibrium. Any exogenous event, which produces a new stable equilibrium, is called “disruption.”

Notes:

1. Using the above definitions it can be said that a disruption is an exogenous event that produces a chronic disease.

2. A disruption is a disturbance with a persisting effect.

Exemplary Assays

1. Take a biological system (e.g., cell, whole organism, etc.). Assay a set of characteristics. Compare the results with the values of the same characteristics in healthy controls. Verify that the system is in healthy equilibrium. Apply a chosen treatment to the system. Following treatment, assay the same characteristics again. If some values deviate from the values of healthy controls, continue to assay these characteristics over time. If the values continue to deviate over time, the treatment produced a chronic disease, and, therefore, can be considered a disruption.

Examples

Genetic knockout, carcinogens, infection with persistent viruses (e.g., HIV, EBV), etc. are disruptions.

(14) Foreign Polynucleotide-Type Disruption (Cause of Disruption)

Definition

Let Pp be a polypeptide. Assume microcompetition with a foreign polynucleotide Pn directly, or indirectly reduces (or increases) Pp bioactivity. A disruption that directly, or indirectly reduces (or increases) Pp bioactivity is called “foreign polynucleotide-type disruption.”

Notes:

1. The first “indirectly” in the definition means that Pp can be downstream from the gene microcompeting with Pn. The second “indirectly” means that Pp can be downstream from the gene, or polypeptide, directly affected by the exogenous event. According to the definition, if both microcompetition with a foreign polynucleotide and an exogenous event increase, or both decrease bioactivity of Pp, the exogenous event can be considered as a foreign polynucleotide-type disruption.

2. Microcompetition with a foreign polynucleotide is a special case of foreign polynucleotide-type disruption.

3. Treatment is a special case of an exogenous event.

4. A foreign polynucleotide-type disruption can first affect a gene or a polypeptide. For instance, a mutation is an effect on a gene. Excessive protein phosphorylation is an effect on a polypeptide.

Exemplary Assays

1. Take a biological system (e.g., cell, whole organism, etc). Assay a set of characteristics. Compare the results with the values of the same characteristics in healthy controls to verify that the system is in a healthy equilibrium. Modify the copy number of Pn, a polynucleotide of interest (by, for instance, transfection, infection, mutation, etc. see above). Identify a gene with modified expression. Assume the assays show decreased expression of G. Take another specimen of the system in healthy equilibrium and apply a chosen treatment to the healthy specimen. Following treatment, assay G expression. Continue to assay G expression over time. If G expression is persistently decreased, the exogenous event can be considered a foreign polynucleotide-type disruption.

Examples

A mutation in the leptin receptor, a mutation in the leptin gene, etc (see more examples below).

(15) Disrupted (Gene, Polypeptide) (Result of Disruption)

Definition

Let Pp be a polypeptide. If a foreign polynucleotide-type disruption modifies (reduces or increases) Pp bioactivity, Pp and the gene encoding Pp are called “disrupted.”

Note: Pp can be downstream from G, the microcompeted gene.

Exemplary Assays

1. Take a biological system (e.g., cell, whole organism, etc). Modify the copy number of Pn, a polynucleotide of interest, (by, from instance, transfection, infection, mutation, etc, see above). Assay bioactivity of genes and polypeptides in the treated system and controls to identify genes and polypeptides with modified bioactivity relative to controls. These genes and polypeptides are disrupted.

Examples

See studies in the section below entitled “Microcompetition with a limiting transcription complex.” See also all GABP regulated genes below.

(16) Disrupted Pathway

Definition

Let the polypeptide Ppₓ be disrupted. A polypeptide Ppᵢ which functions downstream or upstream of Ppₓ, and the gene encoding Ppᵢ, are considered a polypeptide and gene, respectively, in a Ppₓ “disrupted pathway.”

Exemplary Assays

1. Take a biological system (e.g., cell, whole organism, etc). Apply a treatment to the system that modifies Ppᵢ bioactivity. Assay Ppₓ bioactivity. If the bioactivity of Ppₓ changed, Ppᵢ is in a Ppₓ disrupted pathway.

2. Take a biological system (e.g., cell, whole organism, etc). Apply a treatment to the system that modifies Ppₓ bioactivity. Assay Ppᵢ bioactivity. If the bioactivity of Ppᵢ changed, Ppᵢ is in a Ppₓ disrupted pathway.

Examples

See examples below.

(17) Disruptive Pathway

Definition

Consider a polypeptide Ppₖ and a foreign polynucleotide Pn. If a change in bioactivity of Ppₖ increases or decreases Pn copy number, Ppₖ and the gene encoding Ppₖ are considered a polypeptide and a gene in a Pn “disruptive pathway.”

Note: Consider, as an example, microcompetition between a cell and a viral polynucleotide, including the entire viral genome. Ppₖ can be any viral or cellular protein that increase or decreases viral replication.

Exemplary Assays

1. Take a biological system (e.g., cell, whole organism, etc). Apply a treatment to the system that modifies Ppₖ bioactivity, for instance, by increasing expression of a foreign or cellular gene encoding Ppₖ. Assay Pn copy number. If the copy number changed, Ppₖ and the gene encoding Ppₖ, are in a Pn disruptive pathway.

Examples

Consider a GABP virus. The viral proteins that increase viral replication increase the copy number of viral N-boxes in infected cells. According to the definition, these proteins belong to a disruptive pathway. See specific examples below.

b) p300/cbp Related Elements

(1) p300/cbp

Definition

A member of the p300/cAMP response element (CREB) binding protein (CBP) family of proteins is called p300/cbp.

Notes:

1. For reviews on the p300/cbp family of proteins, see, for instance, Vo 2001¹², Blobel 2000¹³, Goodman 2000¹⁴, Hottiger 2000¹⁵ Giordano 1999¹⁶, Eckner 1996¹⁷.

2. CREB binding protein (CBP, or CREBBP) is also called RTS, Rubinstein-Taybi syndrome protein, and RSTS.

3. See sequences of p300/cbp genes and p300/cbp proteins in the List of Sequences below.

Exemplary Assays

1. p300/cbp may be identified using antibodies in binding assays, oligonucleotide probes in hybridization assays, transcription factors such as GABP, NF-κB, E1A in binding assays, etc. (see protocols for binding and hybridization assays below).

Examples

See examples of below.

(2) p300/cbp Polynucleotide

Definition

Assume the polynucleotide Pn binds the transcription complex C. If C contains p300/cbp, Pn is called “p300/cbp polynucleotide.”

Exemplary Assays

1. Take a cell of interest. Modify the copy number of Pn (by, for instance, transfection, infection, mutation, etc, see also above). Use assays described in the section entitled “Identifying a polypeptide bound to DNA or protein complexes,” or similar assays, to test if the protein-Pn complexes contain p300/cbp.

2. See more assays below.

Examples

See below in p300/cbp virus and p300/cbp regulated gene.

(3) p300/cbp Factor

Definition

Assume the transcription factor F binds the complex C. If C contains p300/cbp, F is called “p300/cbp factor.”

Exemplary Assays

1. Use assays describe in the section entitled “Identifying a polypeptide bound to DNA or protein complexes,” or similar assays, to test whether the complexes which contain F also contain p300/cbp.

Examples

The following table lists some cellular and viral p300/cbp factors.

| | | | |
|---|---|---|---|
| p300/cbp | Gene | | |
| factor | symbol | Other names | References |

Cellular

| | | | |
|---|---|---|---|
| AML1 | RUNX1 | Acute myeloid leukemia 1 protein | Kitabayashi 1998¹⁸ |
| | CBFA2 | (AML1); core-binding factor α2 | |
| | AML1 | subunit (CBFα2); oncogene AML-1; | |
| | | Polyomavirus enhancer binding | |
| | | protein 2αB subunit (PEBP2αB); | |
| | | PEA2αB; SL3-3 enhancer factor 1, | |
| | | αB subunit; SL3/AKV core-binding | |
| | | factor αB subunit; SEF1; | |
| | | runt-related transcription factor | |
| | | 1; RUNX1; CBFA2 | |
| A-Myb | MYBL1 | Myb-related protein A; v-myb avian | Facchinetti 1997¹⁹ |
| | AMYB | myeloblastosis viral oncogene | |
| | | homolog-like | |
| ATF1 | ATF1 | Activating transcription factor 1 | Goodman 2000 (ibid) |
| | TREB36 | (ATF1); TREB36 protein; | |
| | | cAMP-dependent transcription factor | |
| | | ATF-1 | |
| ATF2 | ATF2 | Activating transcription factor 2 | Goodman 2000 (ibid), |
| | CREB2 | (ATF2); cAMP response element | Duyndam 1999²⁰ |
| | CREBP1 | binding protein 1 (CRE-BP1); HB16; | |
| | | cAMP-dependent transcription factor | |
| | | ATF-2; TREB7; CREB2 | |
| ATF4 | ATF4 | Activating transcription factor 4 | Goodman 2000 (ibid), |
| | CREB2 | (ATF4); DNA-binding protein | Yukawa 1999²¹ |
| | TAXREB67 | TAXREB67; tax-responsive enhancer | |
| | | element B67 (TAXREB67); TXREB; cAMP | |
| | | response element-binding protein 2 | |
| | | (CREB2); cAMP-dependent | |
| | | transcription factor ATF-4; | |
| | | CCAAT/enhancer binding protein | |
| | | related activating transcription | |
| | | factor (mouse); ApCREB2 (Aplysia) | |
| BRCA1 | BRCA1 | Breast cancer type 1 susceptibility | Goodman 2000 (ibid) |
| | PSCP | protein (BRCA1) | |
| C/EBPβ | CEBPB | CCAAT/enhancer binding protein | Goodman 2000 (ibid), |
| | TCF5 | β (C/EBPβ); nuclear factor | Mink 1997²² |
| | | NF-IL6 (NFIL6); transcription factor | |
| | | 5; CRP2; LAP; IL6DBP; CEBPB; TCF5 | |
| c-Fos | FOS | Proto-oncogene protein c-fos; | Goodman 2000 (ibid), |
| | GOS7 | cellular oncogene fos; G0/G1 switch | Sato 1997 (ibid) |
| | | regulatory protein 7; v-fos FBJ | |
| | | murine osteosarcoma viral oncogene | |
| | | homolog; FOS; G0S7 | |
| C2TA | MHC2TA | MHC class II transactivator; MHC2TA; | Goodman 2000 (ibid), |
| | CIITA | CIITA | Sisk 2000 (ibid) |
| | C2TA | | |
| AP1 | JUN | Transcription factor AP-1; | Goodman 2000 (ibid), |
| | | proto-oncogene c Jun (c-Jun); | Hottiger 2000 (ibid) |
| | | p39; v-jun avian sarcoma virus | |
| | | [symbol] oncogene homolog | |
| c-Myb | MYB | Myb proto-oncogene protein; MYB; | Goodman 2000 (ibid), |
| | | v-myb avian myeloblastosis viral | Hottiger 2000 (ibid) |
| | | oncogene homolo | |
| CREB | CREB1 | cAMP-response-element-binding | Hottiger 2000 (ibid) |
| | | protein (CREB) | |
| CRX | CRX | Cone-rod homeobox (CRX); CRD; cone | Yanagi 2000²³ |
| | CORD2 | rod dystrophy 2 (CORD2) | |
| | CRD | | |
| CID | CI-D | Cubitus interruptus dominant (CID) | Goodman 2000 (ibid) |
| DBP | DBP | D-site binding protein (DBP); | Lamprecht 1999²⁴ |
| | | albumin D bo [symbol] binding protein; | |
| | | D site of albumin promoter (albumin | |
| | | D-box) binding protein; TAXREB302 | |
| E2F1 | E2F1 | Retinoblastoma binding protein 3 | Goodman 2000 (ibid), |
| | RBBP3 | (RBBP-3); PRB-binding protein E2F-1; | Marzio 2000²⁵ |
| | | PBR3; retinoblastoma-associated | |
| | | protein 1 (RBAP- | |
| E2F2 | E2F2 | Transcription factor E2F2 | Marzio 2000 (ibid) |
| E2F3 | E2F3 | Transcription factor E2F3; KIAA0075 | Marzio 2000 (ibid) |
| | KIAA0075 | | |
| Egr1 | EGR1 | Early-growth response factor-1 | Silverman 1998²⁶ |
| | ZNF225 | (Egr1); Krox 24 protein; ZIF268; | |
| | | nerve growth factor-induced protein | |
| | | A; NGFI-A; transcription factor | |
| | | ETR103; zinc finger protein 225 | |
| | | (ZNF225); AT225; TIS8; G0S30; | |
| | | ZIF-268 | |
| ELK1 | ELK1 | Ets-domain protein ELK-1 | Hottiger 2000 (ibid) |
| ERα | ESR1 | Estrogen receptor α (ERα); | Kim 2001²⁷, |
| | NR3A1 | estrogen recepto 1; estradiol | Wang 2001²⁸, |
| | ESR | receptor | Speir 2000²⁹, |
| | | | Hottiger 2000 (ibid) |
| ERβ | ESR2 | Estrogen receptor β; ESR2; NR3A2; | Kobayashi 2000³⁰ |
| | NR3A2 | ESTRB | |
| | ESTRB | | |
| ER81 | | Ets translocation variant 1 (ETV1) | Papoutsopoulou 2000³¹ |
| Ets1 | ETS1 | C-ets-1 protein; v-ets avian | Goodman 2000 (ibid), |
| | | erythroblastosis virus E2 oncogene | Jayaraman 1999³² |
| | | homolog 1; p54 | |
| Ets2 | ETS2 | C-ets-2 protein; human | Jayaraman 1999 (ibid) |
| | | erythroblastosis viru [symbol] oncogene | |
| | | homolog 2; v-ets avian | |
| | | erythroblastosis virus E2 oncogene | |
| | | homolog 2 | |
| GABPα | GABPA | GA binding protein, α subunit | Bannert 1999³³ |
| | E4TF1A | (GABPA); GABP-alpha subunit; | |
| | | transcription factor E4TF1-60; | |
| | | nuclear respiratory factor-2 subunit | |
| | | alpha (NRF-2A) | |
| GABPβ1 | GABPB1 | GA binding protein beta-1 chain | Bannert 1999 (ibid) |
| | GABPB | (GABPB1) [symbol] GABP-beta-1 subunit; | |
| | E4TF1B | transcription factor E4TF1-53; | |
| | | nuclear respiratory factor-2 subunit | |
| | | beta 2 (NRF-2B) | |
| GABPβ2 | GABPB1 | GA binding protein beta-2 chain | Bannert 1999 (ibid) |
| | GABPB | (GABPB2) [symbol] GABP-beta-2 subunit; | |
| | E4TF1B | transcription factor E4TF1-47 | |
| GATA1 | GATA1 | Globin transcription factor 1; | Goodman 2000 (ibid) |
| | GF1 | GATA-bindin [symbol] protein 1 erythroid | |
| | ERYF1 | transcription factor; ERYF1; GF1; | |
| | NFE1 | NF-E1 | |
| Gli3 | GLI3 | Zinc finger protein GLI3; PAP-A; | Goodman 2000 (ibid) |
| | | GCPS; GLI-Kruppel family member | |
| | | GLI3 (Greig cephalopolysyndactyly | |
| | | syndrome); Pallister-Hall syndrome | |
| | | (PHS) | |
| GR | NR3C1 | Glucocorticoid receptor (GR); | Pfitzner 1998 (ibid), |
| | GRL | nuclear receptor subfamily 3, | Hottiger 2000 (ibid) |
| | GCR | group C, member 1 (NR3C1); GRL | |
| HIF1α | HIF1A | Hypoxia-inducible factor-1 α | Goodman 2000 (ibid), |
| | | (HIF1α); ARNT interacting | Bhattacharya 1999³⁴, |
| | | protein; member of PAS protein 1; | Kallio 1998³⁵, |
| | | MOP1 | Ema 1999³⁶, |
| | | | Hottiger 2000 (ibid) |
| HNF4α | HNF4A | Hepatocyte nuclear factor-1 α; | Goodman 2000 (ibid), |
| | NR2A1 | HNF-4-α; transcription factor | Soutoglou 2000³³ |
| | TCF14 | HNF-4; transcription factor 14; | |
| | HNF4 | MODY; maturity onset diabetes o [symbol] | |
| | | the young 1; MODY1; HNF4A; NR2A1; | |
| | | TCF14; HNF | |
| IRF-3 | IRF3 | Interferon regulatory factor-3 | Goodman 2000 (ibid), |
| | | (IRF-3) | Yoneyama 1998³⁸ |
| JunB | JUNB | Transcription factor JunB; proto- | Goodman 2000 (ibid) |
| | | oncogene JunB | |
| Mdm2 | MDM2 | Mouse double minute 2; human homolog | Goodman 2000 (ibid) |
| | | of p53-binding protein (Mdm2); | |
| | | ubiquitin-protein ligase E3 Mdm2; | |
| | | EC 6.3.2.-; p53-binding protein | |
| | | Mdm2; oncoprotein Mdm2; double | |
| | | minute 2 protein; Hdm2 | |
| MEF2C | MEF2C | Myocyte enhancer factor 2C (MEF2C); | Sartorelli 1997 (ibid) |
| | | myocyte-specific enhancer factor 2C; | |
| | | MAD [symbol] box transcription enhancer | |
| | | factor 2 polypeptide C | |
| Mi | MITF | Microphthalmia-associated | Goodman 2000 (ibid), |
| | | transcription factor | Sato 1997³⁹ |
| MyoD | MYOD1M | Myoblast determination protein 1 | Yuan 1996 Ref, |
| | YF3 | (MyoD); myogenic factor MYF-3; | Sartore [symbol] 1997⁴⁰ |
| | | myogenic factor 3; PUM | |
| NF-AT1 | NFAT1 | Nuclear factor of activated T cells, | Garcia-Rodriguez |
| | NFATC2 | cytoplasmic 2; T cell transcription | 1998⁴, Sisk 2000⁴² |
| | NFATP | factor NFAT1; NFAT pre-existing | |
| | | subunit; NF-AT | |
| NF-YB | NFYB | NF-Y protein chain B (NF-YB); | Li 1998⁴³, |
| | HAP3 | nuclear transcription factor Y | Faniello 1999⁴⁴ |
| | | subunit beta; α-CP1, CP1; CCAAT- | |
| | | binding transcription factor subunit | |
| | | A (CBF-A); CAAT-box DNA bindi [symbol] | |
| | | protein subunit B | |
| NF-YA | NFYA | NF-Y protein chain A (NF-YA); CCAAT- | Li 1998 (ibid) |
| | HAP2 | binding transcription factor subunit | |
| | | B (CBF-B); CAAT-box DNA binding | |
| | | protein subuni [symbol] A; nuclear | |
| | | transcription factor Y α | |
| RelA | RELA | NF-κB RelA, transcription factor | Hottiger 1998 (ibid), |
| | NFKB3 | p65; nucle [symbol] factor NF-kappa-B, p65 | Gerritsen 1997⁴⁵, |
| | | subunit; v-rel avian | Speir 2000 (ibid), |
| | | reticuloendotheliosis viral oncogene | Hottiger 2000 (ibid) |
| | | homolo [symbol] A; nuclear factor of kappa | |
| | | light polypeptide gene enhancer in | |
| | | B-cells 3 (p65) | |
| P/CAF | P/CAF | p300/cbp-associated factor | Goodman 2000 (ibid) |
| p/CIP | TRAM-1 | p300/cbp interacting protein | Goodman 2000 (ibid) |
| | NCOA3 | (p/CIP); thyroid hormone receptor | |
| | AIB1 | activator molecule; DJ1049g16.2; | |
| | | nuclear receptor coactivator 3 | |
| | | (thyroid hormone receptor activator | |
| | | molecul [symbol] TRAM-1; receptor-associated | |
| | | coactivator RAC3; amplified in | |
| | | breast cancer AIB1; ACTR | |
| PPARγ | PPARG | Peroxisome proliferator activated | Iannone 2001⁴⁶, |
| | NR1C3 | receptor γ (PPARG); PPAR-gamma; | Kodera 2000⁴⁷ |
| | | PPARG1; PPARG2 | |
| MRG1 | CITED2 | Cbp/p300-interacting transactivator | Bhattacharya 1999 |
| | MRG1 | 2; MSG-related protein 1; | (ibid), Han 2001⁴⁸ |
| | | melanocyte-specific gene 1 MRG1 | |
| | | protein | |
| p45 | NFE2 | Nuclear factor, erythroid-derived 2 | Goodman 2000 (ibid) |
| NF-E2 | | 45 kDa subunit; NF-E2 45 kDa subunit | |
| | | (p45 NF-E2) leucine zipper protein | |
| | | NF-E2 | |
| p53 | TP53 | Cellular tumor antigen p53; tumor | Goodman 2000 (ibid), |
| | P53 | suppresso p53;, phosphoprotein p53; | Avantaggiati 1997⁴⁹ |
| | | Li-Fraumeni syndrome | Van Order 1999⁵⁰, |
| | | | Hottiger 2000 (ibid) |
| p73 | TP73 | Tumor protein p73; p53-like | Goodman 2000 (ibid) |
| | P73 | transcription factor; p53-related | |
| | | protein | |
| Pit-1 | POU1F1 | Pituitary-specific positive | Goodman 2000 (ibid) |
| | PIT1 | transcription facto 1; PIT-1; | |
| | GHF1 | growth hormone factor 1, GHF-1; | |
| | | POU domain, class 1, trans- | |
| | | cription factor 1 | |
| RSK1 | RPS6KA1 | 90-kDA ribosomal S6 kinase, | Goodman 2000 (ibid), |
| | RSK1 | ribosomal protein S6 kinase alpha 1; | Hottiger 2000 (ibid) |
| | | EC 2.7.1.-; S6K-alpha 1; 90 kDa | |
| | | ribosomal protein S6 kinase 1; | |
| | | p90-RSK1;, ribosomal S6 kinase 1; | |
| | | RSK-1 pp90RSK1; HU-1 | |
| RSK3 | RPS6KA2 | Ribosomal protein S6 kinase alpha 2; | Hottiger 2000 (ibid) |
| | RSK3 | EC 2.7.1.-; S6K-alpha 2; 90 kDa | |
| | | ribosomal protein S6 kinase 2;, | |
| | | p90-RSK 2; ribosomal S6 kinase 3; | |
| | | RSK-3; pp90RSK3; HU-2 | |
| RSK2 | RPS6KA3 | Ribosomal protein S6 kinase alpha 3; | Hottiger 2000 (ibid) |
| | RSK2 | EC 2.7.1.-; S6K-alpha 3; 90 kDa | |
| | ISPK1 | ribosomal protein S6 kinase 3; | |
| | | p90-RSK 3; ribosomal [symbol] kinase 2; | |
| | | RSK-2; pp90RSK2; Insulin-stimulated | |
| | | protein kinase 1; ISPK-1; HU-2;, HU-3 | |
| RARγ | RARG | Retinoic acid receptor γ | Hottiger 2000 (ibid), |
| | NR1B3 | (RARγ); retinoic aci receptor | Yang 2001⁵¹ |
| | | gamma-1, RAR-gamma-1; RARC; retinoic | |
| | | acid receptor gamma-2; RAR-gamma-2 | |
| RNA | DDX9 | ATP-dependent RNA helicase A; | Goodman 2000 (ibid) |
| helicase A | NDH2 | nuclear DNA helicase II (NDH II); | |
| | | DEAD-box protein 9; leukophysin (LKP) | |
| RXRα | RXRA | Retinoic acid receptor RXR-α | Goodman 2000 (ibid), |
| | NR2B1 | | Yang 2001 (ibid) |
| ELK4 | ELK4 | ETS-domain protein ELK-4; serum | Goodman 2000 (ibid), |
| | SAP1 | response factor accessory protein | Hottiger 2000 (ibid) |
| | | 1 (SAP-1); SRF accessory protein 1 | |
| SF-1 | NR5A1 | Steroidogenic factor 1 (STF-1, SF-1); | Goodman 2000 (ibid) |
| | FTZF1 | steroi [symbol] hormone receptor AD4BP; | |
| | AD4BP | Fushi tarazu fact [symbol] (Drosophila) | |
| | SF1 | homolog 1; FTZ1; ELP; NR5A (nuclear | |
| | | receptor subfamily 5, group A, | |
| | | member 1) | |
| Smad3 | MADH3 | Mothers against decapentaplegic | Goodman 2000 (ibid), |
| | SMAD3 | (Drosophil homolog 3 (SMAD 3); | Janknecht 1998⁵², |
| | MAD3 | mothers against DPP homolog 3; Mad3; | Feng 1998⁵³, |
| | | hMAD-3; mMad3; JV15-2; hSMAD3 | Pouponnot 1998 (ibid) |
| Smad4 | MADH4 | Mothers against decapentaplegic | de Caestecker⁵⁴, |
| | SMAD4 | (Drosophil homolog 4 (SMAD 4); | Pouponnot 1998 (ibid) |
| | DPC4 | mothers against DPP homolog 4; | |
| | | deletion target in pancreatic | |
| | | carcinoma 4, hSMAD4 | |
| Smad1 | MADH1 | Mothers against decapentaplegic | Pearson 1999⁵⁵, |
| | SMAD1 | (Drosophil homolog 1 (SMAD 1); | Pouponnot 1998⁵⁶ |
| | MADR1 | mothers against DPP homolog 1; | |
| | BSP1 | Mad-related protein 1; transforming | |
| | | growth factor-beta signaling | |
| | | protein-1; BSP-1; hSMAD1; JV4-1 | |
| Smad2 | MADH2 | Mothers against decapentaplegic | Pouponnot 1998 (ibid) |
| | SMAD2 | (Drosophil homolog 2 (SMAD 2); | |
| | MADR2 | mothers against DPP homolog 2; | |
| | | Mad-related protein 2; hMAD-2 [symbol] | |
| | | JV18-1; hSMAD2 | |
| SRC-1 | SRC1 | Steroid receptor coactivtor - 1 | Goodman 2000 (ibid), |
| | NCOA1 | (SRC-1); F-SRC-1; nuclear receptor | Hottiger 2000 (ibid) |
| | | coactivator 1 (NCoA-1); SRC1 | |
| SREBP1 | SREBF1 | Sterol regulatory element binding | Goodman 2000 (ibid), |
| | SREBP1 | protein-1 (SREBP-1); sterol | Oliner 1996⁵⁷ |
| | | regulatory element-bindin | |
| | | transcription factor 1 | |
| SREBP2 | SREBF2 | Sterol regulatory element binding | Goodman 2000 (ibid), |
| | SREBP2 | protein-2 (SREBP-2); sterol | Oliner 1996 (ibid) |
| | | regulatory element-bindin | |
| | | transcription factor 2 | |
| Stat-1 | STAT1 | signal transducer and activator or | Goodman 2000 (ibid), |
| | | transcripti [symbol] - 1α/β; | Paulson 1999⁵⁸, |
| | | transcription factor ISGF-3 | Hottiger 1998 (ibid), |
| | | components p91/p84; signal | Gingras 1999 (ibid), |
| | | transducer and activator of | Zhang 1996⁵⁹ |
| | | transcription 1, 91kD (STAT91) | |
| Stat-2 | STAT2 | Signal transducer and activator or | Goodman 2000 (ibid), |
| | | transcription - 2 (STAT2); ; signal | Paulson 1999 (ibid), |
| | | transduce [symbol] and activator of | Hottiger 1998 (ibid), |
| | | transcription 2, 113kD (STAT113); | Gringras 1999 (ibid), |
| | | p113 | Bhattacharya 1996⁶⁰, |
| | | | Hottiger 2000 (ibid) |
| Stat-3 | STAT3 | Signal transducer and activator or | Paulson 1999 (ibid), |
| | APRF | transcription - 3; acute-phase | Hottiger 1998 (ibid) |
| | | response factor | |
| Stat-4 | STAT4 | Ssignal transducer and activator or | Paulson 1999 (ibid) |
| | | transcription - 4 | |
| Stat-5 | STAT5 | Signal transducer and activator or | Paulson 1999 (ibid) |
| | STAT5A | transcription - 5A (STAT5A); MGF; | check, Gingras 1999 |
| | STAT5B | signal transducer and activator or | (ibid), Pfitzner 1998⁶¹ |
| | | transcription -5B (STAT5B); STAT5 | |
| Stat-6 | STAT6 | Signal transducer and activator or | Paulson 1999 (ibid) |
| | | transcription - 6 (STAT6); IL-4 | check, Gingras 1999⁶² |
| | | Stat; D12S1644 | |
| TAL1 | TAL1 | T-cell acute lymphocytic leukemia-1 | Goodman 2000 (ibid) |
| | SCL | protein TAL-1 protein; STEM cell | |
| | TCL5 | protein; T-cell leukemia/lymphoma-5 | |
| | | protein | |
| TBP | TBP | TATA box binding protein (TBP); | Goodman 2000 (ibid) |
| | TFIID | transcription initiation factor | |
| | TF2D | TFIID; TATA-box factor; TATA | |
| | | sequence-binding protein; SCA17; | |
| | | GTF2D1; HGNC: 15735; GTF2D | |
| TFIIB | TFIIB | Transcription factor IIB (TFIIB, | Goodman 2000 (ibid), |
| | TF2B | TF2B); transcription initiation | Hottiger 2000 (ibid) |
| | GTF2B | factor IIB; general transcription | |
| | | factor IIB (GTFIIB, GTF2B) | |
| THRA | THRA | Thyroid hormone receptor α | Hottiger 2000 (ibid) |
| | NR1A1 | (THRA); C-erbA-alpha; c-erbA-1; | |
| | THRA1 | EAR-7; EAR7; AR7; avian | |
| | ERBA1 | erythroblastic leukemia viral | |
| | | (v-erb-a) oncogene homolog; ERBA; | |
| | | THRA1; THRA THRA3; EAR-7.1/EAR-7.2 | |
| THRB | THRB | Thyroid hormone receptor β1 | Hottiger 2000 (ibid) |
| | NR1A2 | (THRB); thyroid hormone receptor, | |
| | THR1 | beta; avian erythroblastic leukemia | |
| | ERBA2 | viral (v-erb-a) oncogene homolog 2; | |
| | | THRB1; THRB2; ERBA2; NR1A2; thyroid | |
| | | hormone receptor β2 (THRB) | |
| Twist | TWIST | Twist related protein; H-twist; | Goodman 2000 (ibid), |
| | | acrocephalosyndactyly 3 (Saethre- | Hamamori 1999⁶³ |
| | | Chotzen syndrome); twist | |
| | | (Drosophila) homolog; | |
| | | acrocephalosyndactyly 3 (ACS3) | |
| YY1 | YY1 | Ying Yang 1 (YY1); transcriptional | Goodman 2000 (ibid) |
| | | represso protein YY1; delta | |
| | | transcription factor; NF-E1; UCRBP; | |
| | | CF1; Yin Yang 1; DELTA; Y [symbol] | |
| | | transcription factor | |

Viral

| | | |
|---|---|---|
| E1A | | Goodman 2000 (ibid), |
| | | Hottiger 2000 (ibid) |
| EBNA2 | EBV | Goodman 2000 (ibid) |
| Py LT | Polyomavirus large T antigen | Goodman 2000 (ibid) |
| SV40 LT | Simian virus 40 large T antigen, | Goodman 2000 (ibid), |
| | TAg | Hottiger 2000 (ibid) |
| HPV E2 | Human papillomavirus E2 | Goodman 2000 (ibid) |
| HPV E6 | Human papillomavirus E6 | Goodman 2000 (ibid), |
| | | Hottiger 2000 (ibid) |
| Tat | HIV-1 | Goodman 2000 (ibid), |
| | | Hottiger 2000 (ibid) |
| Tax | Human T-cell leukemia virus type 1 | Goodman 2000 (ibid), |
| | | Hottiger 2000 (ibid) |

Bacterial

| | | |
|---|---|---|
| JMY | H pylori (cag) | Goodman 2000 (ibid) |

The two major lists are from reviews by Goodman and Smolik (2000, ibid) and Hottiger and Nabel (2000, ibid).

Mutations in some of these p300 factors are currently associated with chronic diseases, for instance, HNF4A with MODY, ESR1 with breast cancer and bronchial asthma, GR with cortisol resistance, etc. Consider the following definition.

(4) p300/cbp Regulated (Gene, Polypeptide)

Definition

Assume the gene G is transactivated, or suppressed by the transcription complex C. If C contains p300/cbp, the gene G, and the polypeptide encoded by G, are called “p300/cbp regulated.”

Exemplary Assays

1. Co-transfect a cell with the gene promoter fused to a reporter gene, such as CAT or LUC, and a vector expressing p300/cbp. Assay reporter gene expression in the p300/cbp-transfected cell and in control cells transfected with the fused gene promoter along with an “empty” plasmid. If reporter gene expression is higher or lower in the p300/cbp-transfected cell, the gene is p300/cbp regulated.

2. Select a cell that expresses the gene of interest and transfect it with a vector expressing p300/cbp. Assay endogenous gene expression in the p300/cbp-transfected cell and in control cells transfected with an “empty” plasmid. If gene expression is higher or lower in the p300/cbp-transfected cell, the gene is p300/cbp regulated.

Note: Preferably, verify that co-transfection did not induce a change in cellular microcompetition, a mutation in the gene promoter, or a change in methylation of gene promoter.

3. Transfect a cell with the gene promoter fused to a reporter gene, such as CAT or LUC. Contact the cell with an antibody against p300/cbp (or with a protein such as E1A). Assay gene expression in the antibody treated cell and in the untreated controls. If reporter gene expression is higher or lower in the antibody treated cell, the gene is p300/cbp regulated.

4. Select a cell, which expresses a gene of interest. Contact the cell with an antibody against p300/cbp (or with a protein such as E1A). Assay gene expression in both the treated cell and in the untreated controls. If gene expression is higher or lower in the antibody treated cell, the gene is p300/cbp regulated.

5. Perform chromatin assembly of the gene promoter, for instance, with chromatin assembly extract from Drosophila embryos. Add a transcription factor during the chromatin assembly reactions. After the chromatin assembly reaction is complete, add the p300/cbp proteins. Allow time for the interaction of the proteins with the chromatin template. Perform in vitro transcription reaction. Measure the concentration of the RNA products, by for instance, primer extension analysis. Compare to the RNA products before the addition of the p300/cbp proteins. If the addition of p300/cbp increased the concentration of the RNA products, the gene is p300/cbp regulated.

6. See more assays below.

Examples

Direct evidence shows transactivation of certain promoters by p300/cbp (Manning 2001⁶⁴, Kraus 1999⁶⁵, Kraus 1998⁶⁶).

Indirect evidence is available in studies with p300/cbp factors. Consider, for example, the p300/cbp factor GABP. GABP binds promoters and enhancers of many cellular genes including 82 leukocyte integrin (CD18) (Rosmarin 1998⁶⁷), interleukin 16 (IL-16) (Bannert 1999, ibid), interleukin 2 (IL-2) (Avots 1997⁶⁸), interleukin 2 receptor β-chain (IL-2Rβ) (Lin 1993⁶⁹), IL-2 receptor y-chain (IL-2 γc) (Markiewicz 1996⁷⁰), human secretory interleukin-1 receptor antagonist (secretory IL-1ra) (Smith 1998⁷¹), retinoblastoma (Rb) (Sowa 1997⁷²), human thrombopoietin (TPO) (Kamura 1997⁷³), aldose reductase (Wang 1993⁷⁴), neutrophil elastase (NE) (Nuchprayoon 1999⁷⁵, Nuchprayoon 1997⁷⁶), folate binding protein (FBP) (Sadasivan 1994⁷⁷), cytochrome c oxidase subunit Vb (COXVb) (Basu 1993⁷⁸, Sucharov 1995⁷⁹), cytochrome c oxidase subunit IV (Carter 1994⁸⁰, Carter 1992⁸¹), mitochondrial transcription factor A (mtTFA) (Virbasius 1994⁸²), β subunit of the FoF1 ATP synthase (ATPsynβ) (Villena 1998⁸³), prolactin (prl) (Ouyang 1996⁸⁴) and the oxytocin receptor (OTR) (Hoare 1999⁸⁵) among others. For some of these genes, for instance, CD18, COXVb, COXIV, GABP binds to the promoter while for others, for example IL-2 and ATPsynβ, GABP binds an enhancer. More examples see below.

Another p300/cbp factor is NF-Y (see above). Mantovani 1998⁸⁶ provides a list of genes, which include a NF-Y binding site (Mantovani 1998, ibid, Table 1). For the listed genes, the table indicates whether the referenced studies report the presence of a proven binding site for a transcription factor close to the NF-Y binding site, whether cross-competition data with bona fide NF-Y binding sites are available, whether EMSA supershift experiments with anti NF-Y antibodies were performed, and whether the studies performed in vitro or in vivo transactivation studies with NF-Y. Some of the genes listed in the paper are MCH II, Ii, Mig, GP91 Phox, CD10, RAG-1, IL4, Thy-1, globin α, ζ, γ^{D} γ^{P}, Coll α2 (I) α1 (I), osteopontin, BSP, apoA-I, aldolase B, TAT, γ-GT, SDH, fibronectin, arg lyase, factor VIII, factor X, MSP, ALDH, LPL, ExoKII, FAS, TSP-1, FGF-4, α1-chim, Tr Hydr, NaKATPsea-3, PDFGβ, FerH, MHC IA2 B8, Cw2Ld and B7, MDR1, CYP1A1, c-JUN, Grp78, Hsp70, ADH2, GPAT, FPP, HMG, HSS, SREBP2, GHR, CP2, β-actin, TK, TopoIIα, I, II, III, IV, cdc25, cdc2, cyclA, cyclB1, E2F1, PLK, RRR2, HisH2B, HisH3.

(5) p300/cbp Factor Kinase (p300/cbp Factor Phosphatase)

Definition

Assume F is a p300/cbp factor. If a molecule L stimulates phosphorylation or dephosphorylation of F, L is called “p300/cbp factor kinase” or “p300/cbp factor phosphatase,” respectively.

Exemplary Assays

1. Contact a system (for instance, organism, cell, cell lysate, chemical mixture) with a test molecule L. Use assays described in the section entitled “Assaying protein phosphorylation,” or similar assays, to uncover a change in phosphorylation of the p300/cbp factor of interest. An increase in phosphorylation indicates that L is a p300/cbp factor kinase, and a decrease indicates that L is a p300/cbp factor phosphatase.

Example

Ras, Raf, MEK1, MEK2, MEK4, ERK, JNK, three classes of ERK inactivators: type 1/2 serine/threonine phosphatases, such as PP2A, tyrosine-specific phosphatases (also called protein-tyrosine phosphatase, denoted PTP), such as PTP1B, and dual specificity phosphatases, such as MKP-1 which affect phosphorylation of a number of transcription factors, for instance, GABP, NF-κB. See also below.

(6) p300/cbp Agent

Definition

Assume the polynucleotide Pn binds the transcription complex C. Assume C contains p300/cbp. If a molecule L stimulates or suppresses binding of C to Pn, L is called “p300/cbp agent.” Specifically, such an agent can stimulate or suppress binding of p300/cbp to a p300/cbp factor, binding of p300/cbp to DNA, or binding of a p300/cbp factor to DNA.

Exemplary Assays

1. Contact a system (for instance, whole organism, cell, cell lysate, chemical mixture) with a test molecule L. Use assays described in the section entitled “Assaying binding to DNA,” or similar assays, to uncover a change in binding of the C to DNA. Specifically, assay for binding between p300/cbp and DNA, or p300/cbp and a p300/cbp factor, or p300/cbp factor and DNA.

Examples

Examples of p300/cbp agents include sodium butyrate (SB), trichostatin A (TSA), trapoxin (for SB, TSA and trapoxin see in Espinos 1999⁸⁷), phorbol ester (phorbol 12-myristate 13-acetate, PMA, TPA), thapsigargin (for PMA and thapsigargin see Shiraishi 2000⁸⁸, for PMA see Herrera 1998⁸⁹, Stadheim 1998⁹⁰), retinoic acid (RA, vitamin A) (Yen 1999⁹¹), interferon-γ (IFNγ) (Liu 1994⁹², Nishiya 1997⁹³), heregulin (HRG, new differentiation factor, NDF, neuregulin, NRG) (Lessor 1998⁹⁴, Marte 1995⁹⁵, Sepp-Lorenzino 1996⁹⁶, Fiddes 1998⁹⁷), zinc (Zn) (Park 1999⁹⁸, Kiss 1997⁹⁹), copper (Cu) (Wu 1999¹⁰⁰, Samet 1998¹⁰¹, both studies also show phosphorylation of ERK1/2 by Zn), estron, estradiol (Migliaccio 1996¹⁰², Ruzycky 1996¹⁰³, Nuedling 1999¹⁰⁴), interleukin 1β (IL-1β) (Laporte 1999¹⁰⁵, Larsen 1993¹⁰⁶), interleukin 6(IL-6) (Daeipour 1993¹⁰⁷), tumor necrosis factor α (TNFα) (Leonard 1999¹⁰⁸), transforming growth factor β (TGFβ) (Hartsough 1995¹⁰⁹, Yonekura 1999¹¹⁰, oxytocin (OT) (Strakova 1998¹¹¹, Copland 1999¹¹², Hoare 1999, ibid). All studies show phosphorylation of ERK1/2 by these agents. See more agents below.

Other examples include agents that modify oxidative stress, such as, diethyl maleate (DEM), a glutathione (GSH)-depleting agent, and N-acetylcysteine (NAC), an antioxidant and a precursor of GSH synthesis. See more agents below.

(7) Foreign p300/cbp Polynucleotide

Definition

Assume Pn is a polynucleotide foreign to organism R. If Pn is a p300/cbp polynucleotide, Pn is called “p300/cbp polynucleotide foreign to R.”

Exemplary Assays

Combine assays in the p300/cbp polynucleotide and foreign polynucleotide sections above.

Examples

See examples in “p300/cbp virus” below.

(8) p300/cbp Virus

Definition

Assume Pn is a p300/cbp polynucleotide. If Pn is a segment of the genome of a virus V, V is called a “p300/cbp virus.”

Exemplary Assays

1. Verify that Pn is a p300/cbp polynucleotide (see assays above). Compare the sequence of Pn with the sequence of the published V genome. If the sequence is a segment of the V genome, Pn is a p300/cbp virus. If the V genome is not published, its sequence can be determined empirically.

2. Verify that Pn is a p300/cbp polynucleotide (see assays above) by hybridizing Pn to the V genome. If Pn hybridizes, Pn is a p300/cbp virus.

Examples

Direct evidence shows transactivation of certain viruses by p300/cbp. See, for instance, Subramanian 2002¹¹³ on Epstein-Barr virus, Banas 2001¹¹⁴, Deng 2000¹¹⁵ on HIV-1, Cho 2001¹¹⁶ on SV40 and polyomavirus, Wong 1994¹¹⁷, on adenovirus type 5. See also Hottiger 2000 (ibid), a review on viral replication and p300/cbp.

Indirect evidence is available in studies with p300/cbp factors. Consider, for instance, the p300/cbp factor GABP. Since GABP binds p300/cbp (see above), a complex on DNA which includes GABP, also includes p300/cbp. The DNA motif (A/C)GGA(A/T)(G/A), termed the N-box, is the core binding sequence for GABP. The N-box is the core binding sequence of many viral enhancers including the polyomavirus enhancer area 3 (PEA3) (Asano 1990¹¹⁸), adenovirus E1A enhancer (Higashino 1993¹¹⁹), Rous Sarcoma Virus (RSV) enhancer (Laimins 1984¹²⁰), Herpes Simplex Virus 1 (HSV-1) (in the promoter of the immediate early gene ICP4) (LaMarco 1989¹²¹, Douville 1995¹²²), Cytomegalovirus (CMV) (IE-1 enhancer/promoter region) (Boshart 1985¹²³), Moloney Murine Leukemia Virus (Mo-MuLV) enhancer (Gunther 1994¹²⁴), Human Immunodeficiency Virus (HIV) (the two NF-κB binding motifs in the HIV LTR) (Flory 1996¹²⁵), Epstein-Barr virus (EBV) (20 copies of the N-box in the +7421/+8042 oriP/enhancer) (Rawlins 1985¹²⁶) and Human T-cell lymphotropic virus (HTLV) (8 N-boxes in the enhancer (Mauclere 1995¹²⁷) and one N-box in the LTR (Kornfeld 1987¹²⁸)). Moreover, some viral enhancers, for example SV40, lack a precise N-box, but still bind the GABP transcription factor (Bannert 1999, ibid).

Ample evidence exists which supports the binding of GABP to the N-boxes in these viral enhancers. For instance, Flory, et al., (1996, ibid) show binding of GABP to the HIV LTR, Douville, et al., (1995, ibid) show binding of GABP to the promoter of ICP4 of HSV-1, Bruder, et al., 1991¹²⁹ and Bruder, et al., 1989¹³⁰ show binding of GABP to the adenovirus E1A enhancer element I, Ostapchuk, et al., 1986¹³¹ show binding of GABP (called EF-1A in this paper) to the polyomavirus enhancer and Gunther, et al., (1994, ibid) show binding of GABP to Mo-MuLV.

Other studies demonstrate competition between these viral enhancers and enhancers of other viruses. Scholer and Gruss, 1984¹³² show competition between the Moloney Sarcoma Virus (MSV) enhancer and SV40 enhancer and competition between the RSV enhancer and the BK virus enhancer.

Another p300/cbp factor is NF-Y (see above). Mantovani 1998 (ibid) provides a list of viruses that include a NF-Y binding site (Table 1). The list includes HBV S, MSV LTR, RSV LTR, ad EIIL II, Ad MK, CMV gpUL4, HSV IE110k, VZV ORF62, MVM P4.

More Exemplary Assays for Identification of a Polynucleotide Pn as a p300/cbp Polynucleotide:

1. Take a cell of interest. Modify the copy number of Pn in the cell (by, for instance, transfection, infection, mutation, etc, see also above). Assay binding of all p300/cbp factors to Pn. If a p300/cbp factor binds Pn, Pn is a p300/cbp polynucleotide.

2. Assay binding of a p300/cbp factor to endogenous DNA or to exogenous DNA following introduction to the cell of interest. Modify the copy number of Pn in the cell. Assay binding of the p300/cbp factor again. If binding changed, Pn is a p300/cbp polynucleotide.

3. Identify a binding site on Pn for p300/cbp or a p300/cbp factor by computerized sequence analysis.

4. Take a cell of interest. Transfect the cell with a vector that expresses a reporter gene under the control of a promoter of a p300/cbp-regulated gene. Modify the copy number of Pn in the cell (by, for instance, transfection, infection, mutation, etc, see also above). Assay expression of the reporter gene and compare to cells with unmodified copy number of Pn. If expression in the Pn modified cell is different than controls, Pn is a p300/cbp polynucleotide.

5. Take a cell of interest that expresses an endogenous p300/cbp regulated gene. Modify the copy number of Pn in the cell (by, for instance, transfection, infection, mutation, etc, see also above). Assay expression of the p300/cbp-regulated gene and compare to cells with an unmodified copy number of Pn (for instance, in cells transfected with an empty plasmid). If expression in the Pn transfected cell is different than controls, Pn is a p300/cbp polynucleotide.

6. Take a cell of interest. Infect the cell with a p300/cbp virus. Modify the copy number of Pn in the cell (by, for instance, transfection, infection, mutation, etc, see also above). Assay viral replication and compare to cells with unmodified copy number of Pn (for instance, in cells infected with a non p300/cbp virus). If viral replication is different, Pn is a p300/cbp polynucleotide.

7. Compare the sequence of Pn to the genome of a p300/cbp virus using a sequence alignment algorithm such as BLAST. If a segment of the Pn sequence is identical (or homologous) to a segment in viral genome, Pn is a p300/cbp polynucleotide. A polynucleotide of at least 18 nucleotides should be sufficient to ensure specificity and validate alignment.

8. Try to hybridize Pn to the genome of a p300/cbp virus. If Pn hybridizes to the viral genome, Pn is a p300/cbp polynucleotide. Hybridization conditions should be sufficiently stringent to permit specific, but not promiscuous, hybridization. Such conditions are well known in the art.

c) GABP Related Elements

(1) GABP

Definition

A member of the GA binding protein (GABP) family of proteins is called GABP.

Notes:

1. GA binding protein (GABP) is also called Nuclear Respiratory Factor 2 (NRF-2)¹³³, E4 Transcription factor 1 (E4TF1)¹³⁴, and Enhancer Factor 1A (EF-1A)¹³⁵.

2. The literature lists five subunits of GABP: GABPα, GABPβ1, GABPβ2 (together called GABPβ), GABPγ1 and GABPγ2 (together called GABPγ). GABPα is an ets-related DNA-binding protein which binds the DNA motif (A/C)GGA(A/T)(G/A), termed the N-box. GABPα forms a heterocomplex with GABPβ, which stimulates transcription efficiently both in vitro and in vivo. GABPα also forms a heterocomplex with GABPγ, but the heterodimer does not stimulate transcription. The degree of transactivation by GABP appears to correlate with the relative intracellular concentrations of GABPβ and GABPγ. An increase in GABPβ relative to GABPγ increases transcription, while an increase of GABPγ relative to GABPβ decreases transcription. The degree of transactivation by GABP is, therefore, a function of the ratio between GABPβ and GABPγ. Control of this ratio within the cell regulates transcription of genes with binding sites for GABP (Suzuki 1998¹³⁶).

3. See sequences of GABP genes and GABP proteins in the List of Sequences below.

Exemplary Assays

1. GABP may be identified using antibodies in binding assays, oligonucleotide probes in hybridization assays, etc. (see protocols for binding and hybridization assays below).

Examples

See examples below.

(2) GABP Polynucleotide

Definition

Assume the polynucleotide Pn binds the transcription complex C. If C contains GABP, Pn is called a “GABP polynucleotide.”

Exemplary Assays

1. Take a cell of interest. Modify the copy number of Pn (by, for instance, transfection, infection, mutation, etc, see also above). Use assays described in the section entitled “Detailed description of standard elements,” or similar assays, to test if the protein-Pn complexes contain GABP.

2. See more assays below.

Examples

See below in GABP virus and GABP regulated gene.

(3) GABP Regulated (Gene, Polypeptide)

Definition

Assume the gene G is transactivated, or suppressed by the transcription complex C. If C contains GABP, the gene G, and the polypeptide encoded by G, are called “GABP regulated.”

Exemplary Assays

1. Co-transfect a cell with the gene promoter of interest fused to a reporter gene, such as CAT or LUC, and a vector expressing GABP. Assay reporter gene expression in the GABP transfected cell and in control cells transfected with the fused gene promoter along with an “empty” plasmid, that is, with a plasmid identical to the plasmid expressing GABP but without the GABP coding region. If the reporter gene expression is higher or lower in the GABP transfected cell compared to the “empty” plasmid transfected cell, the gene is GABP regulated.

2. Select a cell that endogenously expresses the gene of interest and transfect it with a vector expressing GABP. Assay the gene expression in the GABP transfected cell and in control cells transfected with an “empty” plasmid (see above). If gene expression is higher or lower in the GABP transfected cell compared to the “empty” plasmid transfected cell, the gene is GABP regulated.

Note: Preferably, verify that co-transfection did not induce a change in cellular microcompetition, a mutation in the gene promoter, or a change in methylation of the gene promoter.

3. Transfect a cell with the gene promoter of interest fused to a reporter gene, such as CAT or LUC. Contact the cell with an antibody against GABP. Assay gene expression in the antibody treated cell and untreated controls. If the reporter gene expression is higher or lower in the antibody treated cell compared to the untreated controls, the gene is GABP regulated.

4. Select a cell that expresses a gene of interest. Contact the cell with an antibody against GABP. Assay gene expression in both the treated cell and untreated controls. If gene expression is higher or lower in the antibody treated cell compared to the untreated controls, the gene is GABP regulated.

5. See more assays below.

Examples

GABP binds promoters and enhancers of many cellular genes including (see above). More examples see below.

(4) GABP Kinase (GABP Phosphatase)

Definition

If a molecule L stimulates phosphorylation or dephosphorylation of GABP, L is called “GABP kinase” or “GABP phosphatase,” respectively.

Exemplary Assays

1. Contact a system (for instance, organism, cell, cell lysate, chemical mixture) with a test molecule L. Use assays described in the section entitled “Detailed description of standard elements,” or similar assays, to uncover a change in phosphorylation of GABP. An increase in phosphorylation indicates that L is a GABP kinase; a decrease indicates that L is a GABP phosphatase.

Example

Ras, Raf, MEK1, MEK2, MEK4, ERK, JNK, three classes of ERK inactivators: type 1/2 serine/threonine phosphatases, such as PP2A, tyrosine-specific phosphatases (also called protein-tyrosine phosphatase, denoted PTP), such as PTP1B, and dual specificity phosphatases, such as MKP-1 which affect phosphorylation GABP. See also below.

(5) GABP Agent

Definition

Assume the polynucleotide Pn binds the transcription complex C. Assume C contains GABP. If a molecule L stimulates or suppresses binding of C to Pn, L is called “GABP agent.” Specifically, such agent can stimulate or suppress binding of GABP family members to each other, binding of GABP to other transcription factors, or binding of GABP to DNA.

Exemplary Assays

1. Contact a system (for instance, whole organism, cell, cell lysate, chemical mixture) with a test molecule L. Use assays described in the section entitled “Detailed description of standard elements,” or similar assays, to uncover a change in binding of the complex C to DNA. Specifically, assay binding of GABP family members to each other, binding of GABP to other transcription factors, or binding of GABP to DNA.

Examples

Examples of GABP agents include sodium butyrate (SB), trichostatin A (TSA), trapoxin (for SB, TSA and trapoxin see in Espinos 1999, ibid), phorbol ester (phorbol 12-myristate 13-acetate, PMA, TPA), thapsigargin (for PMA and thapsigargin see Shiraishi 2000, ibid, for PMA see Herrera 1998, ibid, Stadheim 1998, ibid), retinoic acid (RA, vitamin A) (Yen 1999, ibid), interferon-γ (IFNγ) (Liu 1994, ibid, Nishiya 1997, ibid), heregulin (HRG, new differentiation factor, NDF, neuregulin, NRG) (Lessor 1998, ibid, Marte 1995, ibid, Sepp-Lorenzino 1996, ibid, Fiddes 1998, ibid), zinc (Zn) (Park 1999, ibid Kiss 1997, ibid), copper (Cu) (Wu 1999, ibid, Samet 1998, ibid, both studies also show phosphorylation of ERK1/2 by Zn), estron, estradiol (Migliaccio 1996, ibid, Ruzycky 1996, ibid, Nuedling 1999, ibid), interleukin 1β (IL-1β) (Laporte 1999, ibid, Larsen 1998, ibid), interleukin 6 (IL-6) (Daeipour 1993, ibid), tumor necrosis factor α (TNFα) (Leonard 1999, ibid), transforming growth factor β (TGFβ) (Hartsough 1995, ibid, Yonekura 1999, ibid, oxytocin (OT) (Strakova 1998, ibid, Copland 1999, ibid, Hoare 1999, ibid). All studies show phosphorylation of ERK1/2 by these agents. See more agents below.

Other examples include agents that modify oxidative stress, such as, diethyl maleate (DEM), a glutathione (GSH)-depleting agent, and N-acetylcysteine (NAC), an antioxidant and a precursor of GSH synthesis. See details and more agents below.

(6) Foreign GABP Polynucleotide

Definition

Assume Pn is a polynucleotide foreign to organism R. If Pn is a GABP polynucleotide, Pn is called “GABP polynucleotide foreign to R.”

Exemplary Assays

Combine assays in the GABP polynucleotide and foreign polynucleotide sections above.

Examples

See examples in “GABP virus” below.

(7) GABP Virus

Definition

Assume Pn is a GABP polynucleotide. If Pn is a segment of the genome of a virus V, V is called a “GABP virus.”

Exemplary Assays

1. Verify that Pn is a GABP polynucleotide (see assays above). Compare the sequence of Pn with the sequence of the published V genome. If the sequence is a segment of the V genome, Pn is a GABP virus. If the V genome is not published, determine the sequence empirically and compare.

2. Verify that Pn is a GABP polynucleotide (see assays above) by hybridizing Pn to the V genome (see exemplary hybridization assays in the section entitled “Detailed description of standard elements”). If Pn hybridizes, Pn is a GABP virus.

3. Verify that Pn is a GABP polynucleotide by identifying in Pn the DNA motif (A/C)GGA(A/T)(G/A), termed the N-box. Preferably, identify two N-boxes separated by multiples of 0.5 helical turns (HT), up to 3.0 HT (there are 10 base pair per HT) in Pn (see more details below).

Examples

See above. See also below.

More Exemplary Assays for Identification of a Polynucleotide Pn as a GABP Polynucleotide:

1. Take a cell of interest. Assay binding of GABP to endogenous Pn, or exogenous Pn following introduction of Pn to the cell of interest. If a GABP binds Pn, Pn is a GABP polynucleotide.

2. Identify a polynucleotide Pn1 that binds GABP. Assay binding of a GABP to endogenous Pn1, or exogenous Pn1 following introduction of Pn1 to a cell of interest. Modify the copy number of a second polynucleotide, Pn2, in the cell. Assay binding of GABP to Pn1 again. If binding to Pn1 changed, Pn2 is a GABP polynucleotide.

3. Identify a binding site on Pn for GABP by computerized sequence analysis.

4. Take a cell of interest. Transfect the cell with a vector that expresses a reporter gene under the control of a promoter of a GABP regulated gene. Modify the copy number of Pn in the cell (by, for instance, transfection, infection, mutation, etc, see also above). Assay expression of the reporter gene and compare to cells with unmodified copy number of Pn. If expression in the Pn modified cell is different than controls, Pn is a GABP polynucleotide.

5. Take a cell of interest, which expresses an endogenous GABP, regulated gene. Modify the copy number of Pn in the cell (by, for instance, transfection, infection, mutation, etc, see also above). Assay expression of the GABP regulated gene and compare to cells with unmodified copy number of Pn. If expression in the Pn modified cell is different than controls, Pn is a GABP polynucleotide.

6. Take a cell of interest. Infect the cell with a GABP virus. Modify the copy number of Pn in the cell (by, for instance, transfection, infection, mutation, etc, see also above). Assay viral replication and compare to cells with unmodified copy number of Pn (for instance, in cells infected with a non GABP virus). If viral replication is different, Pn is a GABP polynucleotide.

7. Compare the sequence of Pn to the genome of a GABP virus using a sequence alignment algorithm such as BLAST. If a segment of the Pn sequence is identical (or homologous) to a segment in viral genome, Pn is a GABP polynucleotide. A polynucleotide of at least 18 nucleotides should be sufficient to ensure specificity and validate alignment.

8. Try to hybridize Pn to the genome of a GABP virus. If Pn hybridizes to the viral genome, Pn is a GABP polynucleotide. Hybridization conditions should be sufficiently stringent to permit specific, but not promiscuous hybridization. Such conditions are well known in the art.

d) Agents Related Elements

(1) Modulator

Definition

Consider a polynucleotide Pn. An agent, or treatment (called agent for short), is called “modulator” if the agent modifies microcompetition with Pn, modifies at least one effect of microcompetition with Pn, or modifies at least one effect of another foreign polynucleotide-type disruption.

Note: A treatment, such as irradiation, can also be a modulator. In principle, according to the definition, any foreign polynucleotide-type disruption is a modulator.

Exemplary Assays

1. Assay the effect of an agent on Pn copy number.

Specifically, take a biological system (e.g. cell, whole organism, etc). Modify the copy number of Pn (by, for instance, transfection, infection, mutation, etc, see above). Call this cell the Pn cell. Assay the Pn copy number in the Pn cell (see above). Contact the biological system with an agent of interest. Assay again the Pn copy number. If the Pn copy number is higher or lower compared to the copy number in Pn cells not contacted with the agent, the agent is a modulator.

2. Assay the effect of an agent on binding of p300/cbp to Pn; directly or in a complex.

Specifically, take a biological system (e.g. cell, whole organism, etc). Modify the copy number of Pn (by, for instance, transfection, infection, mutation, etc, see above). Call this cell the Pn cell. Assay binding of p300/cbp to Pn (see above). Contact the biological system with an agent of interest. Assay again the binding of p300/cbp to Pn. If the binding is higher or lower compared to binding in Pn cells not contacted with the agent, the agent is a modulator.

3. Assay the effect of an agent on binding of GABP to Pn.

Specifically, take a biological system (e.g. cell, whole organism, etc). Modify the copy number of Pn (by, for instance, transfection, infection, mutation, etc, see above). Call this cell the Pn cell. Assay binding of GABP to Pn (see above). Contact the biological system with an agent of interest. Assay again the binding of GABP to Pn. If binding is higher or lower compared to binding in Pn cells not contacted with the agent, the agent is a modulator.

4. Assay the effect of an agent on binding of p300/cbp to GABP.

Specifically, take a biological system (e.g. cell, whole organism, etc). Modify the copy number of Pn (by, for instance, transfection, infection, mutation, etc, see above). Call this cell the Pn cell. Assay binding of p300/cbp to GABP (see above). Contact the biological system with an agent of interest. Assay again the binding of p300/cbp to GABP. If binding is higher or lower compared to binding in Pn cells not contacted with the agent, the agent is a modulator.

5. Assay the effect of an agent on expression of a disrupted gene and/or polypeptide.

Specifically, take a biological system (e.g. cell, whole organism, etc). Modify the copy number of Pn (by, for instance, transfection, infection, mutation, etc, see above). Call this cell the Pn cell. Identify a disrupted gene and/or polypeptide (see assays above). Contact the biological system with an agent of interest. Assay the bioactivity of the disrupted gene and/or polypeptide. If the bioactivity of the disrupted gene and/or polypeptide is higher or lower compared to the bioactivity in Pn cells not contacted with the agent, the agent is a modulator.

Examples

See below in constructive/disruptive.

(2) Constructive/Disruptive

Definition

A modulator, which attenuates or accentuates microcompetition with a foreign polynucleotide, attenuates or accentuates at least one effect of microcompetition with a foreign polynucleotide, or attenuates or accentuates at least one effect of another foreign polynucleotide-type disruption, is called “constructive” or “disruptive,” respectively.

Notes:

1. A modulator can be both constructive and disruptive.

2. Consider a gene suppressed by microcompetition with a foreign polynucleotide. Consider such a gene in a cell without a foreign polynucleotide. Now consider a mutation, which reduces the gene bioactivity. An agent that stimulates expression of such mutated gene will also be called constructive. If, on the other hand, the mutation stimulates the gene bioactivity, an agent that suppresses its bioactivity will also be called constructive.

3. A constructive agent can be an agonist, if it stimulates expression of a gene suppressed by microcompetition with a foreign polynucleotide, or if is stimulates bioactivity of a polypeptide encoded by such a gene. A constructive agent can also be an antagonist if it inhibits expression of a gene stimulated by microcompetition with a foreign polynucleotide, or inhibits the bioactivity of a polypeptide encoded by such a gene.

4. A foreign polynucleotide-type disruption can be constructive.

Exemplary Assays

1. See assays in Modulator section above. In these assay if either;
(a) Pn copy number in the Pn cell contacted with the agent is higher relative to Pn cells not contacted by the agent;
(b) binding of p300/cbp to Pn in the Pn cell contacted with the agent is higher compared to binding in Pn cells not contacted with the agent;
(c) binding of GABP to Pn in the Pn cell contacted with the agent is higher compared to binding in Pn cells not contacted with the agent;
(d) binding of p300/cbp to GABP in the Pn cell contacted with the agent is higher or lower compared to binding in Pn cells not contacted with the agent;
(e) bioactivity of the disrupted gene and/or polypeptide in the Pn cell contacted with the agent is higher (for genes and/or polypeptides with suppressed bioactivity) compared to the bioactivity in Pn cells not contacted with the agent;
the agent is constructive.

If the effect is in the opposite direction, the agent is disruptive.

Examples

Antiviral drugs, sodium butyrate, garlic, etc. See more examples in Treatment section below.

2. Detailed Description of Standard Elements

a) General Comments

The elements of the present invention may include, as their own elements, standard methods in molecular biology, microbiology, cell biology, transgenic biology, recombinant DNA, immunology, cell culture, pharmacology, and toxicology, well known in the art. The following sections provide details for some standard methods. Complete descriptions are available in the literature. For instance, see the “Current Protocols” series published by John Wiley & Sons. The following list provides a sample of books in the series: Current Protocols in Cell Biology, edited by: Juan S. Bonifacino, Mary Dasso, Jennifer Lippincott-Schwartz, Joe B Harford, and Kenneth M Yamada; Current Protocols in Human Genetics, edited by: Nicholas C Dracopoli, Jonathan L Haines, Bruce R Korf, Cynthia C Morton, Christine E Seidman, JG Seidman, Douglas R Smith; Current Protocols in Immunology, edited by: John E Coligan, Ada M Kruisbeek, David H Margulies, Ethan M Shevach, and Warren Strober; Current Protocols in Molecular Biology, edited by: Frederick M Ausubel, Roger Brent, Robert E Kingston, David D Moore, J G Seidman, John A Smith, and Kevin Struhl; Current Protocols in Nucleic Acid Chemistry, edited by: Serge L Beaucage, Donald E Bergstrom, Gary D Glick, Roger A Jones; Current Protocols in Pharmacology, edited by: S J Enna, Michael Williams, John W Ferkany, Terry Kenakin, Roger D Porsolt, James P Sullivan; Current Protocols in Protein Science, edited by: John E Coligan, Ben M Dunn, Hidde L Ploegh, David W Speicher, Paul T Wingfield; Current Protocols in Toxicology, edited by: Mahin Maines (Editor-in-Chief), Lucio G Costa, Donald J Reed, Shigeru Sassa, I Glenn Sipes. The following lists include more books with standard methods. Basic DNA and RNA Protocols (Methods in Molecular Biology, Vol 58), edited by Adrian J Harwood, Humana Press, 1994; DNA-Protein Interactions: Principles and Protocols (Methods in Molecular Biology, Volume 148), edited by Tom Moss, Humana Press, 2001; Transcription Factor Protocols (Methods in Molecular Biology), edited by Martin J Tymms, Humana Press, 2000; Gene Transcription: A Practical Approach, edited by B D Hames, and S J Higgins, IRL Press at Oxford University Press, 1993; Gene Transcription, DNA Binding Proteins: Essential Techniques, edited by Kevin Docherty, Jossey Bass, 1997; Gene Probes Principles and Protocols (Methods in Molecular Biology, 179), edited by Marilena Aquino de Muro and Ralph Rapley, Humana Press, 2001; Gene Isolation and Mapping Protocols (Methods in Molecular Biology Vol 68), edited by Jackie Boultwood and Jacqueline Boultwood, Humana Press, 1997; Gene Targeting Protocols (Methods in Molecular Biology, Vol 133), edited by Eric B Kmiec and Dieter C Gruenert, Humana Press 2000; Epitope Mapping Protocols (Methods in Molecular Biology, Vol 66), edited by Glenn E Morris, Humana Press, 1996; Protein Targeting Protocols (Methods in Molecular Biology, Vol 88), edited by Roger A Clegg, Humana Press, 1998; Monoclonal Antibody Protocols (Methods in Molecular Biology, 45), edited by William C Davis, Humana Press, 1995; Immunochemical Protocols (Methods in Molecular Biology Vol 80), edited by John D Pound, Humana Press, 1998; Immunoassay Methods and Protocols (Methods in Molecular Biology), edited by Andrey L Ghindilis, Andrey R Pavlov and Plamen B Atanassov, Humana Press, 2002; In situ Hybridization Protocols (Methods in Molecular Biology, 123), edited by Ian A Darby, Humana Presse, 2000; Bioluminescence Methods & Protocols, edited by Robert A Larossa, Humana Press, 1998; Affinity Chromatography: Methods and Protocols (Methods in Molecular Biology), etided by Pascal Bailon, George K Ehrlich, Wen-Jian Fung, wo Berthold and Wolfgang Berthold, Humana Press, 2000; Protocols for Oligonucleotide Conjugates: Synthesis and Analytical Techniques (Methods in Molecular Biology, Vol 26), edited by Sudhir Agrawal, Humana Press, 1993; RNA Isolation and Characterization Protocols (Methods in Molecular Biology, No 86), edited by Ralph Rapley and David L Manning, Humana Press, 1998; Protocols for Oligonucleotides and Analogs: Synthesis and Properties (Methods in Molecular Biology, 20), edited by Sudhir Agrawal, Humana Press, 1993; Basic Cell Culture Protocols (Methods in Molecular Biology, 75), edited by Jeffrey W Pollard and John M Walker, Humana Press, 1997; Quantitative PCR Protocols (Methods in Molecular Medicine, 26), edited by Bernd Kochanowski and Udo Reischl, Humana Press, 1999; In situ PCR Techniques, edited by Omar Bagasra and John Hansen, John Wiley & Sons, 1997; PCR Cloning Protocols: From Molecular Cloning to Genetic Engineering (Methods in Molecular Biology, No 67), edited by Bruce A White, Humana Press, 1996; PRINS and In situ PCR Protocols (Methods in Molecular Biology, 71), edited by John R Gosden, Humana Press, 1996; PCR Protocols: Current Methods and Applications (Methods in Molecular Biology, 15), edited by Bruce A White, Humana Press 1993; Transmembrane Signaling Protocols (Methods in Molecular Biology, Vol 84), edited by Dafna Bar-Sagi, Humana Press, 1998; Chemokine Protocols (Methods in Molecular Biology, 138), edited by Amanda E I Proudfoot, Timothy N C Wells and Chris Power, Humana Press, 2000; Baculovirus Expression Protocols (Methods in Molecular Biology, Vol 39), edited by Christopher D Richardson, Humana Press, 1998; Recombinant Gene Expression Protocols (Methods in Molecular Biology, 62), edited by Rocky S Tuan, Humana Press, 1997; Recombinant Protein Protocols: Detection and Isolation (Methods in Molecular Biology, Vol 63), edited by Rocky S Tuan, Humana Press, 1997; DNA Repair Protocols: Eukaryotic Systems (Methods in Molecular Biology, Vol 113), edited by Daryl S Henderson, Humana Press, 1999; DNA Sequencing Protocols, editors Hugh G Griffin and Annette M Griffin, Humana Press, 1993; Protein Sequencing Protocols (Methods in Molecular Biology, No 64), edited by Bryan John Smith, Humana Press, 2001; Gene Transfer and Expression Protocols (Methods in Molecular Biology, Vol 7), edited by E J Murray, Humana Press, 1991; Transgenesis Techniques, Principles and Protocols (Methods in Molecular Biology, 180), edited by Alan R Clarke, Humana Press, 2002; Regulatory Protein Modification Techniques and Protocols (Neuromethods, 30), edited by Hugh C Hemmings, Humana Press, 1996; Downstream Processing of Proteins Methods and Protocols (Methods in Biotechnology, 9), edited by Mohamed A Desai, Humana Press, 2000; DNA Vaccines Methods and Protocols (Methods in Molecular Medicine, 29), edited by Douglas B Lowrie and Robert Whalen, Humana Press, 1999; DNA Arrays Methods and Protocols (Methods in Molecular Biology, 170), edited by Jang B Rampal, Humana Press, 2001; Drug-DNA Interaction Protocols, editor Keith Fox, Humana Press, 1997; In vitro Mutagenesis Protocols, edited Michael K. Trower, Humana Press, 1996; In vitro Toxicity Testing Protocols (Methods in Molecular Medicine, 43), edited by Sheila O'Hare and C K Atterwill, Humana Press, 1995; Mutation Detection: A Practical Approach (Practical Approach Series (Paper), No 188), edited by Richard G H Cotton, E Edkins and S Forrect, Irl Press, 1998; Herpes Simplex Virus Protocols (Methods in Molecular Medicine, 10), edited by S Moira Brown and Alasdair R MacLean, Humana Press, 1997; HIV Protocols (Methods in Molecular Medicine, 17), edited by Nelson Michael and Jerome H Kim, Humana Press, 1999; Cytomegalovirus Protocols (Methods in Molecular Medicine, 33), edited by John Sinclair, Humana Press, 1999; Antiviral Methods and Protocols (Methods in Molecular Medicine, 24), edited by Derek Kinchington and Raymond F Schinazi, Humana Press, 1999; Epstein-Barr Virus Protocols (Methods in Molecular Biology Vol 174), edited by Joanna B Wilson and Gerhard H W May, Humana Press, 2001; Adenovirus Methods and Protocols (Methods in Molecular Medicine, Vol 21), edited by William S M Wold, Humana Press, 1999; Molecular Methods for Virus Detection, edited by Danny L Wiedbrauk and Daniel H Farkas, Academic Press, 1995; Diagnostic Virology Protocols (Methods in Molecular Medicine, No 12), edited by John R Stephenson and Alan Warnes, Humana Press, 1998. A more extensive list of books with detailed description of standard methods is available at the Promega web site: http://www.promega.com/catalog/category.asp?catalog %5Fname=Promega/5FProducts&category%5Fname=Books&description %5Ftext=Books&Page=1. The Promega list includes 260 books.

For each element, one or more exemplary protocols are presented. All examples included in the application should be considered as illustrations, and, therefore, should not be construed as limiting the invention in any way.

More details regarding the presented exemplary protocols, and details of other protocols that can be used instead of the presented protocols, are available in the cited references, and in the books listed above. The contents of all references cited in the application, including, but not limited to, abstracts, papers, books, published patent applications, issued patents, available in paper format or electronically, are hereby expressly and entirely incorporated by reference.

The following sections first present protocols for formulation of a drug candidate, then protocols, that as elements of above assays, can be used to test a drug candidate for a desired biological activity during drug discovery, development and clinical trials. The assays can also be used for diagnostic purposes. Finally, the following sections also present protocols for effective use of a drug as treatment.

b) Formulation Protocols

One aspect of the invention pertains to administration of a molecule of interest, equivalent molecules, or homologous molecules, isolated from, or substantially free of contaminating molecules, as treatment of a chronic disease.

(1) Definitions

(a) Molecule of Interest

The terms “molecule of interest” or “agent, “is understood to include small molecules, polypeptides, polynucleotides and antibodies, in a form of a pharmaceutical or nutraceutical.

(b) Equivalent Molecules

The term “equivalent molecules” is understood to include molecules having the same or similar activity as the molecule of interest, including, but not limited to, biological activity, chemical activity, pharmacological activity, and therapeutic activity, in vitro or in vivo.

(c) Homologous Molecules

The term “homologous molecules” is understood to include molecules with the same or similar chemical structure as the molecule of interest.

In one exemplary embodiment, homologous molecules may be synthesized by chemical modification of a molecule of interest, for instance, by adding any of a number of chemical groups, including but not limited to, sugars (i.e. glycosylation), phosphates, acetyls, methyls, and lipids. Such derivatives may be derived by the covalent linkage of these or other groups to sites within a molecule of interest, or in the case of polypeptides, to the N-, or C-termini, or polynucleotides, to the 5′ or 3′ ends.

In one exemplary embodiment, homologous polypeptides or homologous polynucleotides include polypeptides or polynucleotides that differ by one or more amino acid, or nucleotides, respectively, from the polypeptide or polynucleotide of interest. The differences may arise from substitutions, deletions, or insertions into the initial sequence, naturally occurring or artificially formulated, in vivo or in vitro. Techniques well known in the art may be applied to introduce mutations, such as point mutations, insertions or deletion, or introduction of premature translational stops, leading to the synthesis of truncated polypeptides. In every case, homologs may show attenuated activities compared to the original molecules, exaggerated activities, or may express a subset or superset of the total activities elicited by the original molecule. In these ways, homologs of constructive or disruptive polypeptides or polynucleotides have biological activities either diminished or expanded compared to the original molecule. In every case, a homolog may, or may not prove more effective in achieving a desired therapeutic effect. Methods for identifying homologous polypeptides or polynucleotides are well known in the art, for instance, molecular hybridization techniques, including, but not limited to, Northern and Southern blot analysis, performed under variable conditions of temperature and salt, can formulate nucleic acid sequences with different levels of stringency. Suitable protocols for identifying homologous polypeptides or polynucleotides are well known in the art (see, for instance, Sambrook 2001¹³⁷ and above listed books of standard protocols). Homologous polypeptides or polynucleotides can also be generated, for instance by a suitable combinatorial approach.

It is well known in the art that the ribonucleotide triplets, termed codons, encoding each amino acid, comprise a set of similar sequences typically differing in their third position. Variations, known as degeneracy, occur naturally, and in practice mean that any given amino acid may be encoded by more than one codon. For instance, the amino acids arginine, serine, and leucine can be encoded by 6 codons. As a result, in one exemplary embodiment, homologous DNA and RNA polynucleotides can be produced which encode the same polypeptide of interest.

In another exemplary embodiment, a set of homologous polypeptides may be generated by incorporating a population of synthetic oligodeoxyribonucleotides into expression vectors already carrying additional portions of the polypeptide of interest. The site into which the oligonucleotide-gene fusion is incorporated must include appropriate transcriptional and translational regulatory sequences flanking the inserted oligonucleotides to permit expression in host cells. Once introduced into an appropriate host cell, the resulting collection of gene-oligonucleotide recombinant vectors expresses polypeptide variants of the polypeptide of interest. The expressed polypeptide may be separately purified by cloning the vector bearing host cells, or by employing appropriate bacteriophage vectors, such as gt-11 or its derivatives, and screening plaques with antibodies against the polypeptide of interest, or against an immunological tag included in the recombinants.

(d) Isolated

The terms “isolated from, or substantially free of contaminating molecules” is understood to include a molecule containing less than about 20% contaminating molecules, based on dry weight calculations, preferably, less than about 5% contaminating molecules.

The terms “isolated” or “purified” do not refer to materials in a natural state, or materials separated into elements without further purification. For example, separating a preparation of nucleic acids by gel electrophoresis, by itself, does not constitute purification unless the individual molecular species are subsequently isolated from the gel matrix.

In one exemplary embodiment, a polynucleotide encoding a polypeptide of interest is ligated into a fusion polynucleotide encoding another polypeptide which facilitates purification, for instance, a polypeptide with readily available antibodies, such as VP6 rotavirus capsid protein, a vaccinia virus capsid protein, or the bacterial GST protein. When expressed, the facilitator polypeptide enables purification of the polypeptide of interest and immunological identification of host cells that express it. In the case of GST-fusion proteins, purification may be achieved by use of glutathione-conjugated sepharose beads in affinity chromatographic techniques well known in the art (see, for instance, Ausubel 1998¹³⁸).

In a related exemplary embodiment, the fusion polypeptide includes a polyamino acid tract, such as the polyhistidine/enterokinase cleavage site, which confers physical properties that inherently enable purification. In this example, purification may be achieved through nickel metal affinity chromatography. Once purified, the polyhistidine tract included to enable purification can be removed by treatment with enterokinase in vitro to release the polypeptide fragment of interest.

For molecules synthesized by an organism, for instance, polypeptides or polynucleotides synthesized by human subjects, in a preferred exemplary embodiment, a purified polynucleotide or polypeptide is free of other molecules synthesized by same organism, accomplished, for example, by expression of a human gene in a non-human host cell.

The following sections present standard protocols for the formulation of certain types of agents.

(2) Small Molecules

One aspect of the invention pertains to administration of a small molecule of interest, equivalent small molecules, or homologous small molecules, isolated from, or substantially free of contaminating molecules, as treatment of a chronic disease.

The following sections present standard protocols for formulation of small molecules.

(a) Production

Small molecules, organic or inorganic, may be synthesized in vitro by any of a number of methods well known in the art. Those small molecules, and others synthesized in vivo, may by purified by, for instance, liquid or thin layer chromatography, high performance liquid chromatography (HPLC), electrophoresis, or some other suitable technique.

(3) Polypeptides

Another aspect of the invention pertains to administration of a polypeptide of interest, equivalent polypeptides, or homologous polypeptides, isolated from, or substantially free of contaminating molecules, as treatment of a chronic disease.

The following sect ions present standard protocols for the formulation of polypeptides.

(a) Production

(i) In Vitro

In one exemplary embodiment, a polypeptide of interest is produced in vitro by introducing into a host cell by any of a number of means well known in the art (see protocols below) a recombinant expression vector carrying a polynucleotide, preferably obtained from vertebrates, especially mammals, encoding a polypeptide of interest, equivalents of such polypeptide, or homologous polypeptides. The recombinant polypeptide is engineered to include a tag to facilitate purification. Such tags include fragments of the GST protein, or polyamino acid tracts either recognized by specific antibodies, or which convey physical properties facilitating purification (see also below). Following culture under suitable conditions, the cells are lysed and the expressed polypeptide purified. Typical culture conditions include appropriate host cells, growth medium, antibiotics, nutrients, and other metabolic byproducts. The expressed polypeptide may be isolated from a host cell lysate, culture medium, or both depending on the expressed polypeptide. Purification may involve any of many techniques well known in the art, including but not limited to, gel filtration, affinity chromatography, gel electrophoresis, ion-exchange chromatography, and others.

Polynucleotides, both mRNA and DNA, can be extracted from prokaryotic or eukaryotic cells, or whole animals, at any developmental stage, for instance, adults, juveniles, or embryos. Polynucleotides may be isolated, or cloned from a genomic library, cDNA library, or freshly isolated nucleic acids, using protocols well known in the art. For instance, total RNA is isolated from cells, and mRNA converted to cDNA using oligo dT primers and viral reverse transcriptase. Alternatively, a polynucleotide of interest may be amplified using PCR. In any case, the initial nucleic acid preparation may include either RNA or DNA and the protocols chosen accordingly. The resulting DNA is, inserted into an appropriate vector, for instance, bacterial plasmid, recombinant virus, cosmid, or bacteriophage, using procedures well known in the art.

Nucleotide sequences are considered functionally linked if one sequence regulates expression of the other. To facilitate expression of a polypeptide of interest, the cloning vector should include suitable transcriptional regulatory sequences well known in the art, for instance, promoter, enhancer, polyadenylation site, etc., functionally linked to the polynucleotide expressing the polypeptide of interest. In one exemplary embodiment, an expression vector is constructed to carry a polynucleotide, a naturally occurring sequence, a gene, a fusion of two or more genes, or some other synthetic variant, under control of a regulatory sequence, such that when introduced into a cell expresses a polypeptide of interest.

Both viral and nonviral gene transfer methods may be used to introduce desirable polynucleotides into cells. Viral methods exploit natural mechanisms for viral attachment and entry into target cells. Nonviral methods take advantage of normal mammalian transmembrane transport mechanisms, for example, endocytosis. Exemplary protocols employ packaging of deliverable polynucleotides in liposomes, encasement in synthetic viral envelopes or poly-lysine, and precipitation with calcium phosphate (see also below).

The variety of suitable expression vectors is vast and growing. For example, mammalian expression vectors typically include prokaryotic elements, which facilitate propagation in the laboratory, eukaryotic elements which promote and regulate expression in mammalian cells, and genes encoding selectable markers. The list of appropriate vectors includes, but is not limited to, pcDNA/neo, pcDNA/amp, pRSVneo, pZIPneo, and a host of others. Many viral derivatives are also available, for instance, pHEBo, derived from the Epstein-Barr virus, BPV-a derived from the bovine papillomavirus, and the pLRCX system (BD Biosciences Clontech, Inc.). The use of mammalian expression vectors is well known in the art (see, for example, Sambrook 2001, ibid, chapters 15 and 16). Similarly, many vectors are available for expression of recombinant polypeptides in yeast, including, but not limited to, YEP24, YEP5, YEP51, pYES2. The use of expression vectors in yeast is well known in the art.

In addition to mammalian and yeast expression systems, a system of vectors is available which permits expression in insect cells. The system, derived from baculoviruses, includes pAcUW-based vectors (for instance, pAcUW1), pVL-based vectors (for instance, pVL1292 and pVL1393), and pBlueBac-based vectors, which carry the gene encoding β-galactosidase to facilitate selection of host cells harboring recombinant vectors.

(ii) In Situ

In another exemplary embodiment, a polypeptide of interest is expressed in situ by administering to an animal or human subject by any of a number of means well known in the art (see protocols below) a recombinant expression vector carrying a polynucleotide encoding the polypeptide of interest, equivalent polypeptides, or homologous polypeptides.

In the present invention, such vectors may be used as therapeutic agents to introduce polynucleotides into cells that express constructive or disruptive polypeptides (for exemplary applications see, for instance, Friedmann 1999¹³⁹).

It is critical that the potential effects of microcompetition between the enhancer, or other polynucleotide sequences carried in the delivery vector, and cellular genes be considered and manipulated where needed. As an example consider a case where the polypeptide of interest binds an enhancer carried by the vector, for instance, a delivery vector that expresses GABP under control of a promoter that includes an N-box. In one exemplary embodiment, the vector expresses, in situ, a high enough concentration of the polypeptide of interest such that any binding of the polypeptide to the enhancer sequences within the vector itself is negligible. In other words, the vector expresses enough free polypeptides to produce the desired biological activity in treated cells. In another example, the polypeptide is not a transcription factor, but the delivery vector carries a polynucleotide that microcompetes with cellular genes for a cellular transcription factor, for instance, a vector that expresses Rb and microcompetes with cellular genes for GABP. In an exemplary embodiment, the delivery vector also includes a polynucleotide sequence that expresses the microcompeted transcription factor, or is delivered in conjunction with another vector that expresses the microcompeted transcription factor. In the example, the Rb vector includes a sequence that expresses GABP, or is delivered in conjunction with a vector that expresses GABP.

(4) Polynucleotides

Another aspect of the invention pertains to administration of a polynucleotide as antisense/antigene, ribozyme, triple helix, homologous nucleic acids, peptide nucleic acids, or microcompetitors, equivalent polynucleotides, or homologous polynucleotides, isolated from, or substantially free of contaminating molecules, as treatment for a chronic disease.

The following sections present standard protocols for the formulation of such polynucleotides. Since antisense/antigene, ribozyme, triple helix, homologous nucleic acids, peptide nucleic acids, and microcompetition agents are nucleic acid based, they share protocols for their synthesis, mechanisms of delivery and potential pitfalls in their use including, but not limited to, susceptibility to extracellular and intracellular nucleases, instability and the potential for nonspecific interactions. In consideration of these common issues, the general methods for the formulation and delivery, as well as caveats regarding the use of nucleic agents, described first, apply similarly to each subsequent agent.

(a) Antisense/Antigene

In the present invention, the terms “antisense” and “antigene” polynucleotides is understood to include naturally or artificially generated polynucleotides capable of in situ binding to RNA or DNA, respectively. Antisense binding to mRNA may modify translation of bound mRNA, while antigene binding to DNA may modify transcription of bound DNA. Antisense/antigene binding may modify binding of a polypeptide of interest to RNA or DNA, for instance binding of an antigene to a foreign N-box may reduce binding of cellular GABP to the foreign N-box resulting in attenuated microcompetition between the foreign polynucleotide and a cellular gene for GABP. Antisense/antigene binding may also modify, i.e., decrease or increase, expression of a polypeptide of interest.

Binding, or hybridization of the antisense/antigene agent, may be achieved by base complementarity, or by interaction with the major groove of the cellular DNA duplex. The techniques and conditions for achieving such interactions are well known in the art.

The target of antisense/antigene agents has been thoroughly studied and is well known in the art. For instance, the antisense preferred target is the translational initiation site of a gene of interest, from approximately 10 nucleotides upstream to approximately 10 nucleotides downstream of the translational initiation site. Oligonucleotides targeting the 3′ untranslated mRNA regions are also effective inhibitors of translation. Therefore, oligonucleotides targeting the 5′ or 3′ UTRs of a polynucleotide of interest may be used as antisense agents to inhibit translation. Antisense agents targeting the coding region are less effective inhibitors of translation but may be used when appropriate.

Effective synthetic agents are typically between 20 and 30 nucleotides in length. However, to be effective, a complementary sequence must be sufficiently complementary to bind tightly and uniquely to the polynucleotide of interest. The degree of complementarity is generally understood by those skilled in the art to be measured relative to the length of the antisense/antigene agent. In other words, three bases of mismatch in a 20 base oligonucleotide have a more profoundly detrimental effect than three bases of mismatch in a 100 base oligonucleotide. Inadequate complementarity results in ineffective inhibition, or unwanted binding to sequences other than the polynucleotide of interest. In the latter case, inadvertent effects may include unwanted inhibition of genes other than a gene of interest. Specificity and binding avidity are easily determined empirically by methods known in the art.

Several methods are suitable for the delivery of antisense/antigene agents. In one exemplary embodiment, a recombinant expression plasmid is engineered to express antisense RNA following introduction into host cells. The RNA is complementary to a unique portion of DNA or mRNA sequence of interest. In an alternative embodiment, chemically derivatized synthetic oligonucleotides are used as antisense/antigene agents. Such oligonucleotides may contain modified nucleotides to attain increased stability once exposed to cellular nucleases. Examples of modified nucleotides include, but are not limited to, nucleotides carrying phosphoramidate, phosphorothioate, and methylphosphonate groups.

Which sequence of the polynucleotide of interest is targeted by antisense/antigene agents, in vitro studies should be undertaken first to determine the effectiveness and specificity of the agent. Control treatments should be included to differentiate between effects specifically elicited by the agent and non-specific biological effects of the treatment. Control polynucleotides should have same length and nucleotide composition as the agent with the base sequence randomized.

Antisense/antigene agents can be oligonucleotides of RNA, DNA, mixtures of both, chemical derivatives of either, and single or double stranded. Nucleotides within the oligonucleotide may carry modifications on the nucleotide base, the sugar or the phosphate backbone. For example, modifications to the nucleotide base involves a number of compounds including, but not limited to, hypoxanthine, xanthine, 2-methyladenine, 2-methyl guanine, 7-methylguanine, 5-fluorouracil, 3-methylcytosine, 2-thiocytosine, 2-thiouracil, 5-methylcytosine, 5-methylaminomethyluracil, and a host of others well known in the art. Modifications are generally incorporated to increase stability, e.g. infer resistance to cellular nucleases, stabilize hybridization, or increase solubility of the agent, increased cellular uptake, or some other appropriate action.

In a related exemplary embodiment, adducts of polypeptides, to target the agent to cellular receptors in vivo, or other compounds which facilitate transport into the target cell are included. Additional compounds may be adducted to the antisense/antigene agent to enable crossing of the blood-brain barrier, cleavage of the target sequence upon binding, or to intercalate in the duplex, which results from hybridization to stabilize that complex. Any such modification, intended to increase effectiveness of the antisense/antigene agent, is included in the present invention.

Similarly, the antisense/antigene agent may include modifications to the phosphate backbone including, but not limited to, phosphorothioates, phosphordamidate, methylphosphonate, and others. The agent may also contain modified sugars including, but not limited variants of arabinose, xylulose, and hexose.

In another exemplary embodiment, the antisense/antigene agent is an alpha anomeric oligonucleotide capable of forming parallel, rather than antiparallel, hybrids with a cellular mRNA of interest.

It is common for antisense agents to be targeted against the coding regions of an RNA of interest to effect translational inhibition. In a preferred embodiment, antisense agents are targeted instead against the transcribed but untranslated region of an RNA transcript. In this case, rather than achieving translational inhibition, it is likely that oligonucleotides hybridized to the target transcript will lead to mRNA degradation through a pathway mediated by RNaseH or similar cellular enzymes.

For optimal efficacy, the antisense/antigene agents must be delivered to cells carrying the polynucleotide of interest in vivo. Several delivery methods are known in the art, including but not limited to, targeting techniques employing polypeptides linked to the antisense/antigene agent that bind to specific cellular receptors. In this instance, the agents may be provided systemically. Alternatively, the agents may be injected directly into the tissue of interest, or packaged in a virus, including retroviruses, chosen because its host range includes the target cell. In every case, the agent must enter the target cell to be effective.

Antisense/antigene methodologies often face the problem of achieving sufficient intracellular concentration of the agent to effectively compete with cellular transcription and/or translation factors. To overcome this challenge, those skilled in the art introduce recombinant expression vectors carrying the antisense/antigene agent. Once introduced into the target cell, expression of the antisense/antigene agent from the incorporated RNA polymerase II or III promoter results in sufficient intracellular concentrations. Vectors can be chosen to integrate into the host cell chromosomes, thereby becoming stable through multiple rounds of cell division, or vectors may be used, which remain, unintegrated and therefore are lost when the target cell divides. In either case, the primary goal is attaining levels of transcription that produce sufficient antisense/antigene agents to be effective. The choice of a suitable vector and the development of an effective antisense construct involves techniques standard in the art.

Antisense/antigene expression man be regulated by any promoter known to be active in mammalian, especially human, cells and may be either constitutively active or inducible. Regardless of the promoter chosen, it is important to test for the effect of any enhancer regions intrinsic to those promoters as they may participate in microcompetition with cellular genes. In the case of inducible promoters, the biological effects of the expressed antisense can be discerned from any effect the promoter has on microcompetition by assaying any bioactivity with and without induced gene expression. Suitable promoters, inducible or not, are well known in the art (see, for example, Jones 1998¹⁴⁰).

Antisense agents may be prepared using any of a number of methods commonly known to those skilled in the art. In on exemplary embodiment, oligonucleotides, up to approximately 50 nucleotides in length, may be synthesized using automated processes employing solid phase, e.g. controlled pore glass (CPG) technology, such as that used on the Applied Biosystems model 394 medium throughput synthesizer, or 5′-phosphate ON (cyanoethyl phosphoramidite) chemistry developed by Clonotech Laboratories, Inc. In each of these procedures, oligonucleotides are synthesized from a single nucleotide using a series of deprotection and ligation steps. The underlying chemistry of the reactions is standard practice and the availability and accessibility of automated synthesizers bring these synthetic technologies within the grasp of anyone skilled in the art.

Despite the ease of synthesis, the selection of effective antisense agents involves the identification of a suitable target for the agent. This process is simplified somewhat by the many software programs available, such as, for example, Premier Primer 5, available from Premier Biosoft International or Primer 3, available online at http://www-genome.wi.mit.edu/cgi-bin/primer/primer3.cgi. Alternatively, a scientist skilled in the art may design antisense agents manually. Relevant aspects of the design process that need attention include selection of the target region to which the antisense agent will bind. Ideally, it will be the gene promoter, if the target is DNA, or the translation initiation site if the target is an mRNA. Attention also needs to be paid to the length of the agent; typically, at least 20 nucleotides are needed for specificity. Shorter oligonucleotides carry the risk of non-specific binding and therefore may lead to undesired side effects. In addition, the agents must be composed of a sequence that will not promote hybridization between the oligonucleotides in the agent during application. Taken together, these considerations are well known and are addressed by standard procedures well known in the art.

Longer antisense agents may be produced within the target cell from recombinant expression vectors. In one exemplary embodiment, the desired antisense-encoding sequences can be incorporated into an appropriate expression vector selected because it contains the regulatory sequences necessary to ensure expression in the target cell type. Selection of the sequence composition of the antisense agent must take into account the same considerations used to design shorter oligonucleotides as described in the previous paragraph including, but not limited to, binding specificity for the target sequence and minimizing interactions between the expressed agents. Techniques for the design and construction of appropriate recombinant expression vectors are well known to those skilled in the art.

Control agents, whether synthetic oligonucleotides or longer antisense agents expressed in vivo by expression vectors, are employed to validate the efficacy and specificity of the therapeutic agents. Each control agent should have the same nucleotide composition and length as the therapeutic agent but the sequence should be random. Employment of this agent will permit the determination of whether any effects observed after treatment with the therapeutic agent are indeed specific. Specificity will reduce the potential for binding to targets other than those desired, thereby reducing associated unwanted side effects.

Purification of Oligonucleotides: The efficacy of synthetic oligonucleotide agents is impacted by their purity. Under typical conditions, approximately 75% of the synthesis products are full length while the remaining 25% of the oligonucleotides are shorter. This proportion of full length to shorter products varies with the length of the desired product. The synthesis of longer oligonucleotides is less efficient, and therefore the synthesis products contain a smaller proportion of full-length products, than that of shorter ones. Unwanted, shorter synthesis products have reduced specificity compared to the full length products and are therefore undesirable in a therapeutic formulation due to their reduced specificity which in turn leads to an increased risk of side effects.

In one exemplary embodiment, full-length oligonucleotides greater than 50 bp in length are purified by virtue of their size. Gel permeation chromatography is used to separate full-length products from the shorter synthetic byproducts. In a complementary exemplary embodiment, full length synthetic oligonucleotides shorter than 50 bp may be purified by liquid chromatography using charged resins such as hydroxyapatite or nucleic acid specific resins such as RPC-5 (which is composed of trioctylmethylamine adsorbed onto hydrophobic plastic particles). This latter technique exploits both hydrophobic and ion exchange methods to achieve high reagent purity and is amenable to use in HPLC.

Regardless of the method of purification used, the desired oligonucleotides are concentrated by precipitation with ice-cold ethanol followed by lyophilization and dissolution in an appropriate carrier for treatment. Carrier selection is another important component of agent formulation. It is essential that the carrier used be first tested for biological activity in the target cell type. This control measure, well known to those skilled in the art, will ensure that any effects observed upon administration of the nucleic acid agent are indeed due to the agent and not the carrier in which it is administered (on purification of oligonucleotides see, for instance, Deshmukh (1999¹⁴¹).

Delivery of Oligonucleotides: Methods for effective administration of antisense agents vary with the agent used. In one exemplary embodiment, synthetic oligonucleotides are delivered by simple diffusion into the target cells. Advantages of this delivery method include the ability to administer the agent systemically, for example by intravenous injection. This method, while effective carries several risks, not the least of which is the potential to introduce oligonucleotides into cells other than those of the desired target. Another disadvantage involves the risk of degradation by nucleases in blood and interstitial fluid. This second disadvantage may be partially avoided by modification of the synthetic oligonucleotide in such a way, for example by incorporated modified nucleotides such as those carrying phosphorothioate or methyl phosphonate moieties, which renders them relatively resistant to exonuclease degradation.

In a related embodiment, the same agents may be delivered by way of liposome-mediated transfection as described by Daftary and Taylor (2001¹⁴²). This method enhances diffusion into the target cell by encasing the antisense agent in a lipophilic liposome. However, this method too has drawbacks. While cellular uptake is enhanced, the ratio of liposome components to DNA must be carefully controlled in order to maximize delivery efficiency. This technique is commonly employed and is well known to those skilled in the art.

In another exemplary embodiment, antisense expressing viral vectors may be used to confer target cell specificity. In some cases, viral delivery agents may be selected which include the target cell type in their respective host range. This delivery method minimizes unwanted side effects that otherwise may arise from delivery of the therapeutic agent to the incorrect cell type. However, this advantage may be negated if the multiplicity of infection is too high and non-specific infection is thereby promoted. This potential problem may be avoided by thoroughly testing any viral deliver agent, using techniques well known in the art, prior to its clinical administration.

(b) Ribozymes

While antisense agents act by either inhibiting transcription or translation of the target gene, or by inducing enzyme-mediated transcript degradation by RNase H or a similar enzyme, ribozymes offer an alternative approach. Ribozymes are RNA molecules that natively bind to and cleave target transcripts. Typical ribozymes bind to and cleave RNA at specific sites, however hammerhead ribozymes cleave target transcripts at sites directed by flanking nucleotide sequences that bind to the target site. The use of hammerhead ribozymes is preferred because the only sequence requirement for their activity is the UG dinucleotide arranged in the 5′-3′ orientation. Hammerhead technologies are well known in the art (see, for example Doherty 2001¹⁴³, or Goodchild 2000¹⁴⁴). In a preferred embodiment, the sequence targeted by the ribozyme lies near the 5′ end of the transcript. That will result cleavage of the transcript near the translation initiation site thereby blocking translation of a full-length protein.

Ribozymes identified in Tetrahymena thermophila, which employ an eight base pair active site which duplexes with the target RNA molecule, are included in this invention. This invention includes those ribozymes, described and characterized by Cech and coworkers (i.e. IVS or L-19IVS RNA), which target eight base-pair sequences in a gene of interest and any others which may be effective in inhibiting expression of a disrupted gene or a gene in a disrupting pathway. For the catalytic sequence of these agents see, for instance, U.S. Pat. No. 5,093,246, incorporated entirely herein by reference. Any ribozyme or hammerhead ribozyme molecules that target RNA sequences expressed by a foreign polynucleotide, disrupted gene or gene in a disrupted pathway, are included in this invention.

Ribozymes, being RNA molecules of specific sequence, may be synthesized with modified nucleotides which enable better targeting to the host cell of interest or which improve stability. As described above for conventional antisense agents, the preferred method of delivery involves introduction into the target cell, a recombinant expression vector encoding the ribosome. Inclusion of an appropriate transcriptional promoter will ensure sufficient expression to cleave and disrupt transcripts of foreign DNA or disrupted genes or genes in a disrupting pathway. The catalytic nature of ribozymes permits their effective use at concentrations below those needed for traditional antisense agents.

Identification of ribozyme cleavage sites within a transcript of interest is accomplished with any of a number of computer algorithms, which scan linear oligonucleotide sequences for alignments with a query sequence. The identified sequence, commonly containing the trinucleotide sequences GUC, GUA, or GUU, will serve as the nucleus of a longer sequence of approximately 20 nucleotides in length. That longer sequence will be examined, again with appropriate computer algorithms well known in the art, for their potential to form secondary structures, which may interfere with the action of targeted ribozyme agents. Alternatively, empirical assays employing ribonucleases may be used to probe the accessibility of identified target sequences.

Ribozymes comprise a unique class of oligonucleotides, which bind to specific ribonucleic acid targets and promote their hydrolysis. The design of ribozyme agents is well known to those skilled in the art. In order to prepare effective ribozyme agents, initially a suitable target sequence must be identified which confers specificity to the agent in order to minimize unwanted side effects and maximize efficacy. Once that target is identified, the ribozyme agent is synthesized using standard oligonucleotide synthesis procedures such as those exemplified herein. Delivery to the target cell may be accomplished by direct transfection ex vivo or by liposome-mediated transfection.

Ensuring the purity and efficacy of ribozyme agents may be more important than for other nucleic acid agents because their intended effects, namely the hydrolysis of target sequences, are irreversible. In this light extensive preclinical testing is essential to minimize unwanted side effects. These risks are, however, outweighed by the potential effectiveness of ribozyme agents.

(c) Triple Helix

In a related embodiment, synthetic single-stranded deoxyribonucleotides can be chosen which form triple helices according to the Hoogsteen base pairing rules. The rules necessitate long stretches of either purines or pyrimidines on one strand of the DNA duplex. In either case, triplexes are formed, with pyrimidines pairing with purines within the target sequence and vice versa, which inhibit transcription of the target sequence. The effectiveness of a targeted triplex forming oligonucleotide may be enhanced by including a “switchback” motif composed of alternating 5′-3′ and 3′-5′ regions of purines and pyrimidines. This “switchback” reduces the length of the required purine or pyrimidine tract in the target because the oligonucleotide can form duplexes alternatively with each strand of the target sequence.

Triple helix forming agents are oligonucleotides that have been designed to interact with cellular nucleic acids and form triple helices. The resulting structure may be targeted by intracellular degradation pathways or may provide a steric block to nucleic acid replication, transcription, or translation depending on the target.

Triplex agent formulation begins with selection of an appropriate target sequence within the cells to be treated. That target may be within the cellular DNA or RNA or within that of an exogenous source such as an infecting virus. Suitable target sequences should contain long stretches of homopyrimidines or homopurines and the most effective targets contain alternative stretches of each. If the target is double stranded DNA, the most effective targets surround and include the transcriptional regulatory regions. Formation of a triplex between the agent and the target will inhibit the binding of RNA polymerase or other requisite transcriptional regulatory factors which otherwise bind the promoter and upstream regulatory regions.

Triplex agents may be synthesized to be more resistant to cellular and extracellular nucleases by the inclusion of modified nucleotides such as those containing phosphorothioate or methyl phosphonate groups. In the event that such modifications interfere with base pairing, additional adducts, such as derivatives of the base intercalating agent acridine, may be incorporated into the therapeutic agent to restore desirable binding properties to the triplex forming oligonucleotide. Alternatively, if the intracellular target is an mRNA, C-5 propyne pyrimidines may be included in the synthetic oligophosphorothioate agent to increase its binding affinity for mRNA and therefore decrease the concentration required for effectiveness.

The affinity of triplex agents for their respective targets may be assessed by electrophoretic gel retardation assays. The formation of triplex structures will retard migration through an electrophoretic gel. Similarly, UV melting experiments can assess the stability of any triplex agent binding to its target. In these assays, triplex agents are mixed with their intended target in vitro and the resulting triplexes are heated (with, for example, a Haake cryothermostat) while monitoring their UV absorbance (with, for example, a Kontron-Uvikon 940 spectrophotometer) (on design of triplex forming oligonucleotides see, for instance, Francois (1999¹⁴⁵)).

Triplex forming agents are simply oligonucleotides designed to form triple helices with the target intracellular nucleic acid. Accordingly, their synthesis, purification, and delivery parallels the procedures described herein for other oligonucleotide agents. Each of these processes is commonly known to those skilled in the art.

(d) Homologous Recombination Agents

Binding of factors to foreign polynucleotides (either DNA or RNA), or polynucleotides of disrupted genes, or polynucleotides of a gene in a disrupted or disrupting pathway, or expression of a foreign gene, or a disrupted gene, or a gene in a disrupted or disrupting pathway can also be reduced by mutating the DNA, inactivating, or “knocking out” the gene or its promoter using targeted homologous recombination.

In one exemplary embodiment, a polynucleotide of interest flanked by DNA homologous to the polynucleotide interest (encompassing either the coding or regulatory regions of the polynucleotide) can be introduced into cells carrying the same sequence. Homologous recombination mediated by the flanking sequences disrupts expression of the polynucleotide of interest and result in reduced expression. The technique is frequently used by those skilled in the art to engineer transgenic animals that produce offspring with same disruption. However, the same approach may be used in humans by administering the engineered construct into target cells. Regardless of expression vector platform chosen, it is important to recognize and control for any microcompetition effects that may be elicited by transcriptional enhancers carried by the viral vectors (see also above). Control experiments must be carried out which study the biological activity of a non-recombinant viral vector to reveal any effects its intrinsic enhancers have on the target biological activities.

Nucleic acid agents for homologous recombination are designed to interact with specific cellular DNA targets and undergo recombination. The specificity of the therapeutic agent is conferred by the nucleotide sequences at its termini; they must be complementary to adjacent cellular targets and bind them through Watson-Crick base pairing.

Formulation of these agents involves careful selection of the desired cellular target. The nucleotide sequence of that target must be available in public or private sequence databases. The agent itself may be comprised of a synthetic oligonucleotide or a recombinant nucleic acid carried in a suitable vector.

In one exemplary embodiment, a synthetic oligonucleotide may be used for homologous recombination in order to interrupt the coding sequence or regulatory sequences of the target gene. The oligonucleotide is designed to include nucleotides at its termini which are complementary to those of the target sequence and the central regions may contain any sequence that is neither complementary to the target sequence nor carry an in-frame insertion into the target sequence.

In a related embodiment, a longer sequence of nucleic acid may be used. The sequence of interest, which is intended to either interrupt a cellular gene or insert additional coding capacity into it, is flanked by sequences homologous to the cellular target. That entire DNA fragment is then inserted into an appropriate prokaryotic or viral vector for delivery to the target cells. Once inside the cell the agent will bind to and recombine with the target gene.

(e) Peptide Nucleic Acids

In various embodiments, hybridization of the nucleic acid agents described herein may be enhanced by the substitution of amino acids for the deoxyribose of the nucleic acid backbone, thereby creating peptide nucleic acids (see, for example, Hyrup 1996¹⁴⁶). This modification leads to a reduction of the overall negative charge on the backbone and therefore reduces the need for counter ions to permit sequence-specific hybridization of two strands of negatively charged polynucleotides. Peptide nucleic acids can be synthesized using techniques well known in the art such as the solid phase protocols described by Hyrup and Nielsen (1996, ibid), and Perry-O'Keefe 1996¹⁴⁷, included herein in their entirety by reference.

Oligonucleotides so modified can be used in the same therapeutic techniques as unmodified homologs. They can be used as antisense agents designed to interfere with the expression of a foreign polynucleotide, a disrupted gene, or a gene in a disrupted pathway. Similarly, by virtue of their enhanced hybridization qualities, peptide nucleic acids can be used, for example, as primers for the PCR, for S1 nuclease mapping of single stranded regions and for other enzyme-based techniques. Similarly, peptide nucleic acids may be modified by the addition of lipophilic moieties to enhance the cellular uptake of therapeutic oligonucleotide agents. In related embodiments, peptide nucleotide agents may be synthesized as chimeras comprised of peptide nucleic acids and unmodified DNA. This configuration exploits the advantages of a peptide nucleic acid while the DNA portion of the molecule can serve as a substrate for cellular enzymes.

Peptide Nucleic Acid (PNA) is a DNA analog in which the sugar-phosphate backbone contains a pseudopeptide rather than the sugars characteristic of DNA. Like DNA, PNA agents bind complementary nucleic acid strands thereby mimicking the behavior of DNA. This activity is enhanced by the neutral, rather than negatively charged, backbone of PNA, which promotes more tenacious and more specific binding than that of DNA. These are among many favorable properties of PNA, include, in addition, increased stability, and exhibit improved hybridization properties compared to their DNA analogs. While the mechanism of PNA action is currently not fully understood, for example PNA-RNA hybrids are not targets for RNase H degradation as are DNA-RNA hybrids, it is likely that they inhibit translation by blocking the binding of RNA polymerase or other critical factors to the target mRNA.

In this light, it is important to select targets that include the translation initiation codon. Other target sites further downstream on the mRNA may be effective at inhibiting translation by interfering with ribosome transit although the role of this activity will need to be determined empirically for each agent developed. In any case the actual mechanism of action, while interesting, is not necessary to ascertain as long as the agent is effective and does not induce undesired side effects.

Homopurines are best targeted by homopyrimidine PNAs with stretches of greater than 8 bp providing suitable targets within double stranded DNA. The synthesis of PNA agents is achieved using automated solid-phase techniques employing Boc-, Fmoc- or Mmt-protected monomers. Alternatively, commercial sources of custom synthetic PNAs, including Applied Biosystems (Foster City, Calif.) may be exploited to minimize in-house expenses and expertise (on design of PNA see, for instance, Nielsen 1999¹⁴⁸).

(5) Antibodies and Antigens

Another aspect of the invention pertains to the administration of an antibody of interest, equivalent of such antibody, homolog of such antibody, as treatment of a chronic disease.

For example, using standard protocols, one skilled in the art can use immunogens derived from a foreign polynucleotide, foreign polypeptide, disrupted gene, disrupted polypeptide, gene or polypeptide in a disruptive or disrupted pathway, to produce anti-protein, anti-peptide antisera, or monoclonal antibodies (see, for example, Harlow and Lane 1999¹⁴⁹, Sambrook 1989¹⁵⁰).

Animals, which have been injected with an immunogenic agent, can serve as sources of antisera containing polyclonal antibodies. Monoclonal antibodies, if desired, may be prepared by isolating lymphocytes from the immunized animals and fusing them, in vitro with immortal, oncogenically transformed cells. Clonal lines from the resulting somatic cell hybrids, or hybridomas, can be used as sources of monoclonal antibodies specific for the immunogen of interest. Techniques for developing hybridomas and for isolating and characterizing monoclonal antibodies are well known in the art (see for instance, Kohler 1975¹⁵¹ and Zola 2000¹⁵²).

In the context of this invention, “antibody” refers to entire molecules or their fragments, which react specifically with polypeptides or polynucleotides of interest, whether they are monospecific, bispecific, or chimeras that recognize more than two antigenic determinants. Those skilled in the art employ well-known methods for producing specific antibodies and for fragmenting them. While several methods are known to produce antibody fragments, pepsin, for example, is used to treat whole antibody molecules to produce F(ab)₂ fragments. These fragments can be further dissociated with chemicals, such as beta mercaptoethanol or dithiothreotol, which reduce intra and intermolecular disulfide bridges resulting in the release of Fab fragments.

Once produced, isolated, and characterized, antibodies, or fragments thereof, which bind to antigenic determinants of interest, may be used for diagnostic and analytical purposes. For example, they may be used in immunohistochemical assays to assess expression levels of polynucleotides or polypeptides of interest. They may also be employed in other immunoassays, including but not limited to, Western blots, immunoaffinity chromatography, and immunoprecipitation carried out to quantify protein levels in cells or tissues of interest. The assays, individually or together, may also be used by one skilled in the art to measure the concentration a protein of interest before and after therapy to assess therapeutic efficacy.

Similarly, it is common in the art to use specific antibodies to screen libraries of recombinant expression vectors for those expressing a protein or polypeptide of interest. Suitable expression vectors are commonly derived from bacteriophage, including, for example, λgt11 and its derivatives. Identification of expression vectors, from among a library of similar recombinants, can lead to the identification of vectors expressing a polypeptide of interest which may then itself be used in diagnostic or therapeutic assays. In a preferred embodiment, antibodies specific for a particular polypeptide, protein or antigenic determinant carried thereon, will cross-react with homologous counterparts from different species to facilitate antibody characterization and assay development.

Antibodies may serve as effective therapeutic agents for the inactivation of specific cellular proteins or for targeting other therapeutic agents to cells expressing particular surface antigens to which an antibody may bind. Polyclonal antibodies are prepared in a suitable host organism, typically rabbit, goat or horse, by injecting the appropriate purified antigen into the host. Following a regimen of repeated challenges by the desired antigen, using protocols well known to those skilled in the art, serum is drawn from the host and assayed for the presence of antibodies. Once a suitable response is detected, additional serum is removed, perhaps leading to exsanguination of the producing organism, and the desired antibodies are purified.

Monoclonal antibodies may be prepared by any number of techniques well known to those skilled in the art. In one exemplary embodiment, cells expressing the desired target antigen are fused with immortalized cells in vitro. The resulting hybridomas are cultured and clonal lines are derived using standard tissue culture techniques. Each resulting clone is assayed for expression of antibodies against the desired antigen, typically but not necessarily by ELISA.

Antibodies may be purified by a number of chromatographic techniques. In one exemplary embodiment, antibodies may be bound to S. aureus protein A cross-linked to a suitable support resin (e.g. sepharose). The crude antibody preparation is slowly applied to the chromatographic column under conditions that permit antibody-protein A interactions. The resin is then washed with several column volumes of buffer to remove adventitiously bound and trapped proteins, leaving only specifically bound antibodies on the column. Those are eluted by washing the column with 100 mM glycine (pH 3.0) and monitoring protein elution spectrophotometrically.

In an alternative embodiment, antibodies are purified by binding to an affinity column comprised of antigen cross-linked to an appropriate solid support. Bound antibodies may be eluted by any of a number of methods and may include the use of an elution buffer containing glycine at low (e.g. 3.0) pH or 3M potassium thiocyanate and 0.5M NH₄OH. Due to the varied mechanisms involved with antibody-antigen interactions, the actual optimal elution conditions must determined empirically.

The therapeutic efficacy of polyclonal compared to monoclonal antibodies cannot be predicted. Each has strengths and weaknesses. For example, polyclonal antibodies necessarily target multiple antigenic determinants on the target antigen. This feature may increase reactivity but, at the same time, may decrease specificity. On the other hand, monoclonal antibodies are exquisitely specific for a single antigenic determinant on the target antigen. This specificity greatly reduces the risk of unwanted reactivity with other antigens, and the associated side effects, yet carries the risk that the target antigenic determinant may be inaccessible in the cellular environment, either due to the natural folding of the protein or through interactions with other cellular molecules. In every case, the efficacy of any antibody agent must be determined empirically using a variety of techniques well known to those skilled in the art.

Antibody production is necessarily preceded by the isolation and purification of appropriate antigens. Cellular proteins may be purified by any of a number of techniques well known to those skilled in the art. In one exemplary embodiment, cells expressing the desired antigen are lysed in the presence of non-ionic detergents and the resulting lysate is subjected to purification. That lysate is then fractionated by precipitation in the presence of ammonium sulfate. Sequentially higher concentrations of ammonium sulfate are used to derive protein mixtures that differ by their solubility in ammonium sulfate. Each fraction is then assessed for the presence of the desired antigen.

The fraction carrying the protein of interest is subjected to further purification by any of a number of well-known methods. For instance, if an antibody against the protein is available, the protein may be purified by affinity chromatography using a resin of substrate, typically sepharose, dextran or some similar insoluble polymer, to which the antibody is conjugated. The protein mixture containing the desired antigen is exposed to the resin under conditions that promote antibody-antigen interactions. Adventitiously bound proteins are washed from the resin with an excess of binding buffer and the antigens are eluted with buffer containing an ionic detergent such as sodium dodecylsulfate (SDS).

In an alternative embodiment, crude fractions of cellular proteins are further purified using methods well known in the art involving ion exchange or molecular exclusion chromatographic techniques. The purity of antigens isolated by any technique may be assessed by electrophoresis through denaturing polyacrylamide gels followed by visualization by staining.

c) Assay Protocols

One aspect of the invention pertains to assaying the effect of an agent on a molecule of interest, equivalent molecules, or homologous molecules during drug discovery, development, use as treatment, or during diagnosis.

(1) Definitions

(a) Molecule of Interest

The term “molecule of interest” is understood to include, but not limited to, p300/cbp, p300/cbp polynucleotides, p300/cbp factors, p300/cbp regulated genes, p300/cbp regulated polypeptides, p300/cbp factor kinases, p300/cbp factor phosphatases, p300/cbp agents, foreign p300/cbp polynucleotides, p300/cbp viruses, disrupted genes, disrupted polypeptides, genes in disrupted pathways, polypeptides in disrupted pathways, genes in disruptive pathways, polypeptides in disruptive pathways.

Every gene and protein mentioned in this invention is uniquely defined by its sequence as published in public databases. See, for instance, the sequences in the nucleotide and protein sequence databases at NCBI (also known as Entrez, the name of the search and retrieval system), GenBank, the NIH genetic sequence database, DDBJ, the DNA DataBank of Japan, EMBL, the European Molecular Biology Laboratory database (GenBank, DDBJ and EMBL comprise the International Nucleotide Sequence Database Collaboration), SWISS-PROT, the protein knowledgebase, and TrEMBL, the computer-annotated supplement to SWISS-PROT (see also the search and retrieval system Expasy), PROSITE, the database of protein families and domains, and TRANSFAC, the database of transcription factors. By a gene it is meant the coding and non-coding regions, the promoters, enhancers, and the 5′ and 3′ UTRs. Published sequences are considered standard information and are well known in the art. In one exemplary embodiment, sequences for certain genes and proteins of interest in this invention are listed in the following section. For most genes, the list includes the human sequence. However, homologous sequences (see definition below) are available in the above databases for other organisms, such as mouse, rat, etc. The following listed sequences should be regarded as illustrations, and, therefore, should not be construed as limiting the invention in any way.

List of Sequences

Metallothionein IIA (J00271, V00594, X97260, S52379, P02795)
Interferon gamma (AF330164)
Platelet-derived growth factor B chain (PDGFB) (Y14326, XM_{—}009997)
Platelet-derived growth factor alpha polypeptide (PDGFA) (NM_{—}002607)
Neuregulin 1 (NRG1) (NM_{—}013964)
Heregulin-beta1 (M94166)
TNF-alpha (AB048818)
TNF-beta (Lymphotoxin) (D12614)
Oxytocin receptor (OXTR) (NM_{—}000916, X80282 M25650)
Kappa light chain nuclear factor, NFKB (L01459)
Selectin P (NM_{—}003005)
Selectin E (NM_{—}000450)
Integrin, alpha (NM_{—}000885)
Hormone-sensitive lipase (NM_{—}005357)
TGF-beta 1 (A18277)
ICAM-1 (X84737)
GM-CSF (AJ224149)
CD8 antigen (NM_{—}004931)
CD11A antigen, integrin alpha L (XM_{—}008099)
CD11b (NM_{—}000632)
CD11C (NM_{—}000887)
CD28 glycoprotein (AH002636)
CD34 antigen (CD34) (NM_{—}001773)
CD40 (XM_{—}009624)
CD40 ligand (X67878 S50586)
CD44 (NT_{—}024229)
CD54 (NT_{—}011130 NT_{—}004939)
CD58 (XM_{—}001325)
CD62L (NT_{—}004939)
CD69 antigen (BC007037)
CD80 antigen (CD28 antigen ligand 1, B7-1 antigen) (XM_{—}002948)
CD86 antigen (CD28 antigen ligand 2, B7-2 antigen) (XM_{—}002802)
Interleukin 1, beta (IL1B) (NM_{—}000576)
Interleukin 1 receptor antagonist (IL1-RA) (XM_{—}010756 P18510 NM_{—}000577 AJ005835 BC009745 M55646 M63099 X52015 X53296 X64532 X84348 AF043143)
Interleukin 2 (IL2) (AF359939)
Interleukin 2 receptor, beta (IL2R) (XM_{—}009962)
Interleukin 4 (IL4) (AF395008)
Interleukin 5 (IL5) (AF353265)
Interleukin 6 (IL6) (AF048692)
Interleukin 10 (IL10) (XM_{—}001409)
Interleukin 12A (NM_{—}000882)
Interleukin 12B (NM_{—}002187)
Interleukin 13 (IL13) (AF377331)
Interleukin 16 (NM_{—}004513)
Aldose reductase (BC010391)
Neutrophil elastase (AC004799)
Folate binding protein (FBP) (X62753)
Cytochrome c oxidase subunit Vb (Cox Vb) (M1996¹)
Cytochrome c oxidse subunit IV (Cox IV) (BC008704)
Transcription factor A, mitochondrial (TFAM) (NM_{—}012251)
ATP synthase beta (NM_{—}001686)
Prolactin (PRL) (XM_{—}004269)
Retinoic acid receptor, beta (RARB) (XM_{—}003071)
Choline acetyltransferase (CHAT) (XM_{—}011848)
Cholinergic receptor, nicotinic, beta polypeptide 4 (CHRNB4) (NM_{—}000750)
RAF1 (NM_{—}002880)
Nicotinic acetylcholine receptor (AChR) (X17104)
Acetylcholine receptor delta subunit (X55019 X53091 X53516)
Cholinergic receptor, nicotinic, epsilon polypeptide (XM_{—}008520)
PKC alpha (X52479)
v-Ha-ras (XM_{—}006146)
v-fos FBJ murine osteosarcoma viral oncogene homolog (FOS) (NM_{—}005252)
Cytochrome P450 monoxygenase CYP2J2 (U37143)
Fibronectin (E01162)
Vascular cell adhesion molecule 1 (VCAM-1) (X53051)
PECAM1 (NM_{—}000442)
MCP-1 (Y18933)
AP-2 (X77343)
Apob-100 (M14162)
Actin, beta (ACTB) (XM_{—}004814)
GAPDH (NT_{—}009731)
Cyclin-dependent kinase 4 (CDK4) (NM_{—}000075)
Cyclin-dependent kinase 2 (CDK2) (XM_{—}006726)
Human cyclin D1 (M64349)
Human cyclin D2 (X68452)
Human cyclin A1 (NM_{—}003914)
Skeletal muscle alpha-actin (ACTA1) (AF182035)
Retinoic acid receptor, alpha (BC008727)
Transforming growth factor-beta (TGF-beta) (X02812 J05114)
Beta-1-adrenergic receptor (ADRB1) (AF169007)
Adrenergic, beta-2-, receptor, surface (ADRB2) (NM_{—}000024)
Insulin (BC005255)
Leptin (Lep) (U65742)
Leptin receptor db form (OB-Rdb) (U58863)
Myelin basic protein (MBP) (XM_{—}008797)
RANTES (AF088219)
MIP-1 alpha/RANTES receptor (E13385)
MIP-1 beta (NT_{—}010795)
Chemokine (C—C motif) receptor 5 (CCR5) (NM_{—}000579)
Thioredoxin (TXN) (XM_{—}015718)
Thrombopoietin (XM_{—}002815)
Polyomavirus (NC_{—}001515 NC_{—}001516)
JC virus (J02226 J02227 NC_{—}001699)
SV40 (J02400 J02402-3 J02406-10 J04139 M24874 M24914 M28728 V01380 NC_{—}001669)
BK virus (NC_{—}001538 V01108 J02038 strain dunlop V01109 J02039 strain MM J02038 K00058 V01108 strain dunlop M23122 strain AS)
Lymphotropic polyomavirus (K02562)
Human adenovirus type 2 (NC_{—}001405)
Human adenovirus 5 (NC_{—}001406 M73260 M29978)
Human adenovirus type 5 E1A enhancer (M13156)
Human adenovirus 17 (NC_{—}002067 AF108105)
Human adenovirus 40 (L19443)
Human herpesvirus 1 (NC_{—}001806 X14112 D00317 D00374 S40593)
Human herpesvirus 2 (NC_{—}001798)
Human herpesvirus 3 (NC_{—}001348)
Human herpesvirus 4 (NC_{—}001345)
Human herpesvirus 5 (NC_{—}001347 X04650 D00328 D00327 X17403 (strain AD169) M17956 M21295 U33331 D63854 K01263 M60321 X03922 M1129 M18921)
Human herpesvirus 6 (NC_{—}001664 X83413 (U1102, variant A) AB021506 (variant B, strain HST))
Human herpesvirus 6B (NC_{—}000898 AF157706 L13162 L14772 L16947 (strain Z29))
Human herpesvirus 7 (NC_{—}001716 U43400 (JI) AF037218 (strain RK))
Epstein-Barr virus (EBV) (V01555 J02070 K01729-30 V01554 X00498-99 X00784 (strain B95-8) L07923 X58140 D10059)
Rous sarcoma virus (NC_{—}001407)
Y73 sarcoma virus (NC_{—}001404)
Human coxsackievirus A (NC_{—}001429)
Coxsackievirus B3 (NC_{—}001473)
Moloney murine leukemia virus (NC_{—}001501 J02255 J02256 J02257 M76668 AF033811)
Human immunodeficiency virus type 1 (AJ006022 NC 001802 K02013 K03455 M38432 AF286239 U86780 AF256211 AF256205 AF256207 AF256206 X04415 K03456)
Human immunodeficiency virus type 2 (NC_{—}001722 J04542 U27200 L14545 D00835 U38293 X05291 M31113 X52223 M15390 J04498 M30502 U22047 L07625 M30895 D00477 X61240 X16109 AF082339)
Human T-cell lymphotropic virus type 1 (AF033817 NC_{—}001436 AF259264 U19949 AF042071 J02029 M33896 AF139170 L03561)
Human T-cell lymphotropic virus type 2 (AF326584 NC_{—}001488 AF326583 AF139382 Y13051 Y14365 AF074965 NC_{—}001877)
LCMV (Y16308 M20869 M22138 AF079517 AF186080 AJ233196 AJ297484 AJ233200 AJ233161 AH004719 AH004717 AH004715 S75753 S75741 S75739 912860 912868)
TMEV (NC_{—}001366 AF030574 M80890 M80889 M80888 M80887 M80886 M80885 M80884 M80883 M16020 M14703 M20562 M20301 M94868)
Hepatitis B virus (NC_{—}001707 AF330110 AB042283 AB042282 AB050018 AB042284 AB049609 AB049610 AF182803 AB042285 AF182804 AF182805 AF182802 AF384371 AF363961 AF384372)
Collagen type 1 alpha2 (COL1A2) (M35391 K02568 AF004877 AC002528 M22817 M20904 XM_{—}004658 Z74616 L47668 NM_{—}000089 M22816 M20904 J03464 Ml 8057 X02488 M21671 Y00724 V00503 S89896 M64229 S96821 AB004317 L00613 U79752 S62614 S59218 S59211 S89898 X67667 P08123)
Collagen type 1 alpha 1 (COL1A1) (XM_{—}037910 AF017178)
Tissue factor (XM_{—}001322 J02931 J02681 NM_{—}001993 M16553 J02846 M27436 AL138758 A19048 P13726 P30931 AAB20755 KFBO3 X53521 KFRB3 P24055 AAA63469 CAA37597 AAF36523 Q9JLU8 M26071 AAA40414 KFMS3 NP_{—}034301 P20352 AAA63400 AAA16966 P42533 NP_{—}037189)
Integrin, beta 2 (CD18) (X64074 X63835 X64075 X63835 X64076 X63835 X64077 X63835×64078 X63835 X64079 X63835 X64080 X63835 X64081 X63835 X64082 X63835×64083 X63835 X63924 X63835 X63925 X63835 X63926 X63835 X64073 X63835 AL163300 AP001755 BA000005 BC005861 S81234 Y00057 M19545 M15395 NM_{—}000211 X64071 X63835 X63926 X63835 AH003850 S81231 S81252 S81247 S75381 S75297 M95293 M38701 X54481 M77675 PO₅₁₀₇)
Rb1 (L11910 M27845 M27846 M27847 M27848 M27849 M27850 M27851 L35146 M27852 M27853 M27854 M27855 M27856 M27857 M27858 M27859 M27860 L35147 M27862 M27863 M27864 M27865 M27866 X16439 L41890 L41891 L41893 L41894 L41895 L41896 L41897 L41898 L41899 L41997 L41999 L41907 L41914 L41904 L41921 L41996 L41998 L42000 L41911 L41924 L41923 L41920 L41918 L41870 L49209 L49212 L49213 L49218 L49220 L49223 L49230 L49231 L49232 AH006304 AH005289 AH005290 AH005288 M26460 M28736 M15400 M28419 M33647 J02994 NM_{—}000321 AF043224 XM_{—}007211 M19701 J03809 AAA53483)
BRCA1 (U37574 XM_{—}008213 XM_{—}008214 XM_{—}008215 XM_{—}008216 XM_{—}008217 XM_{—}008219 XM_{—}008220 XM_{—}008221 XM_{—}008222 XM_{—}017568 XM_{—}017569 XM_{—}017570 NM_{—}007294 NM_{—}007295 NM_{—}007296 NM_{—}007297 NM_{—}007298 NM_{—}007299 NM_{—}007300 NM_{—}007301 NM_{—}007302 NM_{—}007303 NM_{—}007304 NM_{—}007305 NM_{—}007306 U14680 AF005068 U68041 U64805 Y08864 XP_{—}017569 XP_{—}008212)
Fas (X63717 NM_{—}000043 X83493 X89101 Z47993 Z47994 Z47995 Z70519 Z70520 P25445)
p300 (XM_{—}010013 U01877 NM_{—}001429 Q09472 S67605 AL096765)
CREB-binding protein (CBP) (AC004760 NP_{—}004371 AJ251844 U47741 U85962 U89354 U89355 XM_{—}036668 XM_{—}036667 XM_{—}036669 BG710081 S66385 U88570)
ZF_TAZ matrix, p300/cbp protein binding site (PS50134 XM_{—}017011 XM_{—}009709 XM_{—}017011 AF078104 M74515 M74511 AF057717)
E4 TF1-60 (D13318 X84366)
E4 TF1-53 (D13317)
E4 TF1-47 (D13316)
Human nuclear respiratory factor-2 subunit alpha (U13044)
Human nuclear respiratory factor-2 subunit beta 1 (U13045)
Human nuclear respiratory factor-2 subunit beta 2 (U13046)
Human nuclear respiratory factor-2 subunit gamma 2 (U13048)
GA-binding protein, subunit beta 1 (NM_{—}005254 NM_{—}016654 BC004103 M74516 M74512)
GA-binding protein, subunit beta 2 (NM_{—}002041 NM_{—}016655 M74517 M74513)
GA-binding protein, subunit gamma 1 (U13047)
Ets1 (J04101 X14798 NM_{—}005238 M11921 XM_{—}015368 XP_{—}015368)
ERK1 (AJ222708 NM 002745 M84490 BC000205 Z11696 S38872 P27361 Z11694 S38867 Z11695 S38869)
ERK2 (M84489 P28482)
JNK1 beta 2 (U35005)
JNK1 beta 1 (U35004)
JNK2 beta 2 (U35003)
JNK2 beta 1 (U35002)
JNK1 alpha 2 (U34822)
JNK2 alpha 1 (U34821)
JNK3 alpha 1 (U34820)
JNK3 alpha 2 (U34819)
JNK2 (L31951)
JNK1 beta 2 (AAC50611)
MEK1 (L05624 NM_{—}002755 Q02750)
MEK kinase 1 (MEKK1) (AF042838)
MEK kinase 3 (MEKK3) (U78876)
Human STAT1 (P42224 NM_{—}007315 AF182311 BC002704 M97936 U18662 U18663 U18664 U18665 U18666, U18667 U18668 U18669 U18670)
Human STAT2 (U18671 M97934 S81491 P52630)
Human IL-2 receptor, gamma (NM_{—}000206 D11086 L12183 AC087668 L19546 P31785)
Alpha 2 adrenergic receptor (MI 8415)
Beta 3 adrenergic receptor (P13945 X72861)
Beta 3 adrenergic receptor X70811)
Beta 3 adrenergic receptor (X70812)
Beta 3 adrenergic receptor (S53291)
CCAAT/enhancer binding protein (C/EBP)(NM_{—}005194)
Cbp/p300-interacting transactivator (BC004240)
AML1 (AF312387 AF025841 AF312386 AY004251)
AML (D10570)
AML1 (D43967 D43969 D89788 D89789 D89790 L21756 L34598 M83215 U19601 X79549 X90976 X90978 X90981 AP001721 Q01196)
A-Myb (X66087 S75881 X13294 P10243)
ATF1 (X55544)
ATF2 (P15336 AY029364 M31630 U16028 X15875)
ATF4 (P18848 AL022312 BC008090 BC011994 D90209 M86842)
c-Fos (P01100 AB022276 AF111167 BC004490 K00650 V01512)
AP1 (P05412 AL136985 BC002646 BC006175 BC009874 J04111)
C2TA (P33076 AF410154 U18259 U18288 U31931 X74301)
c-Myb (P10242 AF104863 M13665 M13666 M15024 U22376 X52125 P17676 AL161937 BC005132 BC007538 X52560 P16220 BC010636 M27691 M34356 S72459 X555450
CREB (X60003 O431860
CRX (AF024711)
CID (P19538)
DBP (Q10586 BC011965 D28468 U06936 U48213 U792830
E2F1 (Q01094 AF086380 AL121906 BC005098 M96577 S49592 S74230 U47675 U47677)
E2F2 (Q14209 AL021154 L22846)
E2F3 (O00716 AL136303 D38550 Y10479)
Egr1 (P18146 AJ243425 M62829 M80583 X52541)
ELK1 (P19419 AB016193 AB016194 AF000672 AF080615 AF080616 AL009172 M25269)
Ets2 (P15036 AF017257 AL163278 AP001732 J04102 M11922 X55181)
ER81 (P50549 AC004857 U17163 X87175 P03372 AF120105 AF172068 AF172069 AF258449 AF258450 AF258451 AL078582 AL356311 M12674 S80316 U476780
ER alpha (X03635 X624620
ER beta (Q92731 AB006589 AB006590 AF051427 AF051428 AF060555 AF061054 AF061055 AF074598 AF074599 AF124790 AF215937 X99101).
GATA1 (P15976 AF196971 BC009797 M30601 X17254)
Gli3 (P10071 AC005028 AJ250408 M20674 M57609 PO₄₁₅₀ AC005601 BC015610 M109010
GR (M69104 M73816 U01351 U80946 X03225 X03348 Q16665 AF050127 AF207601 AF207602 AF2084870
HIF1A (AF304431 BC012527 U22431 U29165 U85044 X72726)
HNF4A (P41235 AL132772 U72967 X76930 X87870 X87871 X87872 Z49825)
JunB (P17275 BC004250 BC009465 BC009466 M29039 U20734 X51345)
MDM2 (Q00987 AF201370 AF385322 AF385323 AF385324 AF385326 AF385327 AJ276888 AJ278975 AJ278976 AJ278977 AJ278978 BC009893 M92424 U33199 U33200 U33201 U33202 U33203 Z12020 NM_{—}006878 NM_{—}006879 NM_{—}006880 NM_{—}006881 NM_{—}006882)
MDMD2 (AF385325)
MEF2C (Q06413 L08895 S57212)
Mi (O75030 AB006909 AB009608 AB032357 AB032358 AB032359 AL110195 Z29678)
MyoD (P15172 AF027148 BC000353 X17650 X56677)
RelA (Q04206 BC011603 BC014095 L19067 M62399 Z22948 Z22951)
NFAT1 (Q13469 AL035682 U43341 U43342)
NF-YB (P25208 BC005316 BC005317 BC007035 L06145 X59710)
NF-YA (P23511 NM_{—}021705 AK025201 AL031778 M59079 X59711)
P/CAF (Q92831)
p/CIP (Q9Y6Q9 AL0344180 Q9UPG4)
MRG1 (Q99967 AF109161 AF129290 BC004377 U65093)
NFE2 (Q16621 BC005044 L13974 L24122 S77763 P04637 AF052180 AF066082 AF135121 AF136271 AF307851 BC003596 K03199M13121 M14694 M14695 M22881 M22898 U94788 X01405 X02469 X541560 X60010 X600110
p53 (X60012 X60013 X60014 X60015 X60016 X60017 X60018 X60019 X60020)
p73 (O15350 AF077628 AL136528 Y11416)
RSK1 (NM_{—}002953 AL109743 BC014966 L07597 Q15418)
RSK3 (AL022069 AX019387 BC002363 L07598 X85106)
RSK2 (P51812 L07599 U08316)
PIT1 (P28069 D10216 D12892 L18781 X62429 X72215)
RARG (P13631 AJ250835 L12060 M24857 M38258 M57707 P22932)
RXRA (AF052092 BC007925 BC009882 U66306 X52773 Q08211 L13848 U03643 Y10658 P28324 NM_{—}001973 M85164 M85165 Q13285 D842060
SF-1 (D84207 D84208 D842090 D84210 D88155 U76388 Q13485 AF0454470
SMAD4 (BC002379 U44378 Q15797 BC001878 U548260
SMAD1 (U57456 U59423 U59912)
SMAD2 (Q15796 AF027964 BC014840 U59911 U65019 U68018 U78733)
SMAD3 (Q92940 U68019 U76622)
SRC1 (AJ000882 NM_{—}0037430 AJ000881 U19177 U19179 U40396 U59302 U90661)
SREBP1 (P36956 U00968)
SREBP2 (Q12772 U02031 Z99716)
STAT3 (P40763 AJ012463 BC000627 BC014482 L29277)
STAT4 (Q14765)
STAT5A (P42229 L41142 U43185)
STAT5B (P51692 U47686 U48730 P42226 AF067572 AF067573 AF067574 AF067575 BC004973 BC0058230
STAT6 (U16031 U66574)
TAL1 (P17542 AJ131016 AL135960 M29038 M61108 S53245 X51990)
TBP (P20226 AL031259 M34960 M55654 X54993)
TF2B (Q00403 AL445991 S44184)
THRA (P10827 BC000261 BC002728 J03239 M24748 M24899 X55005 X55074 Y00479)
THRB (P10828 M26747 X04707 P37243)
TWIST (Q15672 U80998 X91662 X99268 Y10871)
IRF3 (Q14653 AF112181 AX015330 AX015339 BC009395 U86636 Z56281)
YY1 (P25490 AF047455 M76541 M77698 Z14077)
PPARG (P37231 NM_{—}015869 BC006811 D83233 L40904 U63415 U79012 X90563)
AR (P10275 AF162704 L29496 M20132 M20260 M21748 M23263 M27430 M34233 M35851 M58158 S79366 S79368 M27424 M27425 M27426 M27427 M27428 M27429 M35845 M35846 M35847 M35848 M35849 M35850)
SRD5A1 (P18405 AF052126 AF113128 AL008713 BC006373 BC007033 BC008673 M32313 M68886 M68882 M68883 M68884 M68885 AF073302 AF073304)

(b) Equivalent Molecules

The term “equivalent molecules” is understood to include molecules having the same or similar activity as the molecule of interest, including, but not limited to, biological activity and chemical activity, in vitro or in vivo.

(c) Homologous Molecules

The term “homologous molecules” is understood to include molecules with the same or similar chemical structure as the molecule of interest (see exemplary embodiments above).

The following section presents standard assays, which can be used, in conjunction with the assays in the new elements section, to test the effect of an agent on a molecule of interest.

(d) During

The term “during drug discovery, development, use as treatment, or during diagnosis” is understood to include, but not be limited to, drug screening, rational design, optimization, in laboratory or clinical trials, in vitro or in vivo (see exemplary embodiment below).

(2) Assaying Protein Concentration

(a) UV Absorbance

In one exemplary, embodiment, cellular protein concentration is measured by virtue of its absorbance of ultraviolet light at the wavelength of 280 nm (Ausubel 1999¹⁵³); To calibrate the reagents used, and to validate the spectrophotometer, a standard curve is established using protein solutions of known concentration. Typically solutions of bovine serum albumin, a commonly available protein, are used to establish the standard curve. Cells are lysed in a detergent-rich buffer to liberate membrane associated and intracellular proteins. Following lysis, insoluble materials are removed by centrifugation. The absorbance of UV light by the supernatant, which contains soluble proteins of unknown concentration, is then measured and compared to the standard curve. Comparison of the data obtained from the cellular extracts with those represented by the standard curve provides an indication of cellular protein concentration.

(b) Bradford Method

In another exemplary embodiment, protein concentration is determined using the Bradford method (Sapan 1999¹⁵⁴, Ausubel 1999, Ibid). A standard curve is constructed using solutions of known protein concentration mixed with coomassie brilliant blue. Following a brief incubation at room temperature, the absorbance of light at 595 nm is measured and a standard curve is constructed. Cells are lysed as described above; the lysate is mixed with coomassie brilliant blue and the absorbance measured in a manner identical to that of the standard curve. Comparison of the values obtained from the cellular extract with those of the solutions of known concentration reveals the concentration of cellular proteins.

(c) Immunoaffinity Chromatography

To measure concentration of a specific cellular protein, for instance, p300, GABP or CBP, additional steps are employed to purify the protein away from other cellular proteins. One exemplary embodiment involves the use of specific antibodies targeted against the protein of interest to remove it from the cellular lysate. Specific antibodies, for instance, anti-p300, anti-GABP, or anti-CBP, are chemically bound to a resin and contained within a vertical glass or plastic column. Cell lysate is passed over that resin to permit antibody-antigen interactions, thereby allowing the protein to bind to the immobilized antibodies. Efficient removal of the protein of interest from the cell lysate is accomplished by using an excess of antibody. Protein bound to the column is removed which releases the bound protein. The eluted protein is collected and its concentration determined by an assay for protein concentration such as those exemplified above.

(3) Assaying mRNA Concentration

(a) UV Absorbance

In certain embodiments, RNA concentration is measured by absorption of ultraviolet light at a wavelength of 260nm (Manchester 1995¹⁵⁵, Davis 1986¹⁵⁶, Ausubel 1999, Ibid). RNA is purified from cells by first lysing the cells in a detergent rich buffer. Proteins in the cellular lysate are degraded by incubation overnight at 65° C. with proteinase K. After enzymatic degradation, proteins are extracted from the solution by mixing with phenol/chloroform/isoamyl alcohol followed by extraction with chloroform/isoamyl alcohol. Nucleic acids in the resulting protein deficient solution are precipitated by addition of salt, typically sodium acetate or ammonium acetate, and ethanol. After a brief incubation of the mixture at −20° C., the insoluble nucleic acids are removed by centrifugation, dried, and redissolved in a sterile, RNase free solution of Tris and EDTA. Contaminating DNA is removed from the lysate by treatment with RNase-free DNase I. Degraded DNA is removed by precipitation of the intact RNA with salt and ethanol. The dried, purified RNA is dissolved in Tris-EDTA and quantified by virtue of its absorbance of light at 260 nm. Since the molar extinction coefficient of RNA at 260 nm is well known, the concentration of RNA in the solution can be determined directly.

(b) Northern Blot

The concentration of a particular RNA species can also be determined. In one exemplary embodiment, the amount of mRNA which encodes a protein of interest, for instance, p300, GABP, CBP, within a population of cells is measured by Northern blot analysis (Ausubel 1999, Ibid, Gizard 2001¹⁵⁷). Total cellular RNA is isolated and separated by electrophoresis through agarose under denaturing conditions, typically in a gel containing formaldehyde. The RNA is then transferred to, and immobilized upon a charged nylon membrane. The membrane is incubated with a solution of detergent and excess of low molecular weight DNA, typically isolated from salmon sperm, to prevent adventitious binding of the gene specific, for instance, p300-, GABP-, CBP-specific, radiolabeled DNA probe to the membrane. Radiolabeled cDNA probes representing the protein, e.g., p300, GABP, CBP, are then hybridized to the membranes and bound probe is visualized by autoradiography.

(c) Reverse Transcriptase—Polymerase Chain Reaction (RT-PCR)

In another exemplary embodiment, the amount of mRNA encoding a protein of interest, for instance, p300, GABP, CBP, expressed by a population of cells is measured by first isolating RNA from cells and preparing cDNA by binding oligo deoxythymidine (dT) to the polyadenylated mRNA within the prepared RNA. Reverse transcriptase is then used to extend the bound oligo dT primers in the presence of all four deoxynucleotides to create DNA copies of the mRNA. The cDNA population is then amplified by the polymerase chain reaction in the presence of oligonucleotide primers specific for the sequence of the gene or RNA of interest and Taq DNA polymerase. The amplification product scan be visualized by gel electrophoresis followed by staining with ethidium bromide and exposure to ultraviolet light. Quantification can be achieved by adding a radiolabeled deoxynucleotide to the PCR reaction. Radiolabel incorporated into the amplification products is visualized by autoradiography and quantified by densitometric analysis of the autoradiograph or by direct phosphorimager analysis of the electrophoretic gel.

(d) S1 Nuclease Protection

In a related exemplary embodiment, expression of RNA encoding a protein of interest, for instance, p300, GABP, CBP, can be assessed by hybridizing isolated cellular RNA with a radiolableled synthetic DNA sequence homologous to the 5′ terminus of the RNA of the protein of interest. The synthetic deoxyribonucleotide, less than 40 nucleotides in length, is labeled at it 5′ end with T4 polynucleotide kinase and γ-³²P ATP. Once the oligonucleotide is bound to the RNA, the mixture is incubated in the presence of the single strand-specific nuclease S1. Any unhybridized, and therefore single stranded, molecules of RNA or DNA are degraded, leaving the DNA-RNA hybrids of the protein of interest intact. The undegraded hybrids are removed from the solution by precipitation with ammonium acetate and ethanol and resolved by nondenaturing gel electrophoresis. Radiolabeled bands on the gel are then visualized by autoradiography. The radiolabel can be quantified by densitometric analysis of the autoradiographs or by phosphorimager analysis of the electrophoretic gels themselves.

(4) Assaying Polynucleotide Copy Number

(a) S1 Nuclease Protection

This same technique can be used to quantify the level of any nucleic acid, naturally expressed or exogenous, within a population of cells. In every case, the sequence of the single stranded synthetic oligonucleotide must be designed so that it is complementary to the 5′ terminal sequence of the species to be measured.

(b) Real Time PCR

In another exemplary embodiment, DNA copy number can be measured using real time PCR (Heid 1996¹⁵⁸). This technique employs oligonucleotides doubly labeled. At the 5′ ends they carry a reporter dye that fluoresces upon excitation by the appropriate wavelength of light. At the 3′ end they carry a quencher dye that suppresses the fluorescence of the first dye. These oligonucleotides are prepared so that their sequence is complementary to the region of interest, which lies between the forward and reverse PCR primers. Once hybridized to the DNA sequence of interest, the close proximity of the quencher dye and the fluorescent dye suppresses the fluorescent emissions of the reporter dye. However, during the process of PCR, Taq polymerase cleaves the reporter dye from the oligonucleotide and releases it. Once removed from the nearby quencher dye, fluorescence is permitted. Free fluorescent dye is quantified with a fluorimeter and is directly related to the number of molecules of interest present prior to PCR.

(5) Detection of Binding

(a) General

In one exemplary embodiment, an assay to identify compounds that bind to a polynucleotide or polypeptide of interest involves binding of a test compound to wells of a microtiter plate by covalent or non-covalent binding. For instance, the assay may anchor a specific test compound to a microtiter plate substrate using a mono or polyclonal immobilized antibody. A solution of the test compound can also be used to coat the solid surface. Then, the nonimmobilized polynucleotide or polypeptide of interest may be added to the surface coated wells. After sufficient time is allowed for the reaction to complete, the residual components are removed by, for instance, washing. Care should be taken not to remove complexes anchored on the solid surface. Anchored complexes may be detected by several methods known in the art. For instance, if the nonimmobilized polynucleotide or polypeptide of interest, or test compound were labeled before the reaction, the label may be used to detect the anchored complexes. If the components were not prelabeled, a label may be added during or after complex formation, for instance, an antibody directed against the nonimmobilized polynucleotide or polypeptide of interest, or test compound, can be added to the surface coated wells.

In a variation of this assay, the polynucleotide or polypeptide of interest is anchored to a solid surface and the nonimmobilized test compound is added to the surface coated wells.

In another variation of this assay, the reactions are performed in a liquid phase, and the complexes are removed from the reaction mixture by immunoaffinity chromatography, or immunoprecipitation, as described herein.

(b) Detection of Binding to DNA

In one exemplary embodiment, DNA fragments carrying a known, or suspected binding domain for a polypeptide of interest, for instance, p300, GABP, etc., are purified by gel electrophoresis and labeled with T4 polynucleotide kinase in the presence of γ³²P-ATP (Bulman et al. 2001). Labeled DNA is then added to a solution containing the polypeptide of interest under conditions, ionic and thermal, which permit formation of DNA-polypeptide complexes. The solution is then maintained for a period of time sufficient for the reaction to complete. Following completion, the mixture is separated by electrophoresis through nondenaturing polyacrylamide in parallel to labeled, but otherwise unreacted test DNA. Following electrophoresis, the labeled DNA is detected by autoradiography or by phosphorimager analysis. Formation of complexes is detected by the shift in electrophoretic mobility (see also below).

The assay detects polypeptide-DNA complexes formed by direct binding of the polypeptide of interest with DNA, or by indirect binding through intermediary polypeptides, as long as the intermediary polypeptides are present in the reaction mixture. Further, the magnitude of the gel shift provides a semi-quantitative measure of the relative concentration of the polypeptide-DNA binding in the assay mixture. As such, changes in concentration can also be detected.

(i) Affinity Chromatography

In one exemplary embodiment, binding of a polypeptide of interest, that is, disrupted polypeptide, or polypeptide in a disrupted or disruptive pathway, such as p300, GABP, CBP, to DNA is measured by first expressing fragments of the polypeptide of interest as GST (glutathione sulfonyl transferase) fusion proteins in E. Coli (Gizard 2001, Ibid). The expressed polypeptides are then bound to glutathione coupled sepharose. Radiolabeled DNA fragments, carrying ³²P, representing the polypeptide binding site, are incubated with protein-bead complexes and subsequently washed three times to remove adventitiously bound DNA. Any. DNA bound to the immobilized polypeptide of interest is released by boiling in presence of the ionic detergent SDS. Liberated radiolabeled DNA is quantified by liquid scintillation counting, or by direct measurement of Cerenkov radiation.

(ii) Electrophoretic Gel Mobility Shift Assay

In another exemplary embodiment, binding of a polypeptide of interest, or a group of polypeptides to DNA is assessed by electrophoretic gel mobility shift assay (Gizard 2001, Ibid, Ausubel 1999, Ibid, Nuchprayoon 1999¹⁵⁹). Radiolabeled DNA carrying the polypeptide binding site, for instance, the p300 binding site, or N-box, is mixed with the recombinant polypeptide, for instance, p300, GABP, expressed as GST fusion protein. Subsequent resolution by electrophoresis through nondenaturing polyacrylamide gels in parallel with labeled DNA alone reveals a shift in electrophoretic mobility only if the polypeptide is bound to DNA in the DNA/polypeptide mixtures. If the DNA binding site is unknown, or one is suspected to be carried in a collection of DNA fragments, this assay can be performed to test for, and potentially affirm the presence of such a binding site.

(6) Detection of Binding Interference

A polynucleotide or polypeptide of interest may bind with one or many cellular or extracellular proteins in vivo. Compounds that interfere with, or disrupt the binding may include, but are not limited to, antisense oligonucleotides, antibodies, peptides, and similar molecules.

In one exemplary embodiment, binding interference of a test compound is assessed by adding the compound to a mixture containing a polynucleotide or polypeptide of interest and a binding partner. After enough time is allowed for the reaction to be completed, the complex concentration in the test reaction mixture is compared to a control mixture prepared without the test compound, or with a placebo. A decreased concentration in the test reaction indicates interference. Reactants may be added at different orders regardless of the method used. For example, a test compound may be added to the reaction mixture before adding the polynucleotide or polypeptide of interest and their binding partners, or at the same time. A test compound that can disrupt an already formed complex, for instance, by displacing a complex component, can be added to the reaction mixture after complex formation. The interference assay can be conducted in two ways, in liquid, or in solid phases, as described above.

In another embodiment, a polynucleotide or polypeptide of interest is prepared for immobilization by fusion to glutathione-S-transferase (GST), while maintaining the binding capacity of the fusion protein. Another complex component, a cellular polynucleotide or polypeptide, or extracellular protein, can be purified, and then utilized in developing a monoclonal antibody using methods well known in the art. The GST-polynucleotide fusion protein is coupled to glutathione-agarose beads and exposed to the other complex component in the presence or absence of a test compound. After sufficient time has been allowed for the reaction to complete, unbound components are removed, for instance, by washing, and the labeled monoclonal antibody is added. Bound radiolabeled antibody is then measured to quantify the extent of complex formation. Inhibition of complex formation by a test compound decreases measured radioactivity. As above, a test compound capable of complex disruption can also be added after complex formation.

In one variation of the assay, the fusion protein is mixed with the other complex component in liquid, that is, without solid glutathione-agarose beads.

In another variation of the assay, peptide fragments of the binding domains, instead of full-length complex components are used. Several methods well known in the art can be used to identify and isolate binding domains. For instance, one method-entails mutating a gene and screening for a disruption in normal binding of the polypeptide encoded by the gene by co-immunoprecipitation or immunoaffinity. If the polypeptide shows disrupted binding, analysis of the gene sequence can reveal the binding domain, or the region of the polypeptide involved in binding. Another approach partially proteolyzes a labeled polypeptide anchored to a solid surface. Non-bound fragments are removed by washing leaving a labeled polypeptide comprising the binding domain immobilized on the solid surface. The polypeptide fragments bound to the immobilized proteins are than isolated and analyzed by amino acid sequencing, using for instance the Edman degradation procedure (Creighton 1983¹⁶⁰) Another approach expresses specific fragments of a polynucleotide, or gene, and tests the fragments for binding activity.

In another embodiment, an assay uses a complex with one component labeled. However, binding to the complex quenches the signal generated by the label (see, for instance, U.S. Pat. No. 4,109,496). A test compound that disrupts the complex, for instance, by displacing a part of the complex, restores the signal. This assay can be used to identify compounds, which either interfere with complex formation, or disrupt an already formed complex.

Specifically, a test compound can interfere with binding between a disrupted gene or polypeptide, or a gene or polypeptide in a disruptive or disrupted pathway, for instance, a microcompeted or mutated gene or polypeptide, and their binding partner. The assay may be especially useful in identifying compounds capable of interfering in binding reactions between foreign polynucleotides and cellular polypeptides without interfering in binding between cellular polynucleotide and cellular polypeptides. The assay is also especially useful in identifying compounds capable of interfering in binding between mutant cellular polynucleotide, or polypeptide, and normal cellular polynucleotide, or polypeptide, without interfering in binding between normal polynucleotide or polypeptides.

(7) Identification of a Polypeptide Bound to DNA or Protein Complex

(a) Immunoprecipitation

In one exemplary embodiment, the identity of a bound polypeptide, for instance, p300, GABP, CBP, is confirmed by reacting antibodies specific to the polypeptide of interest with polypeptides bound to DNA. For example, p300-specific antibodies are mixed with the polypeptide-DNA complexes and incubated overnight at 4° C. Immune complexes are then precipitated by the addition of a secondary antibody directed against the primary p300-specific antibody. Precipitated antibody-antigen complexes are resolved by denaturing gel electrophoresis and the constituent proteins are visualized by staining with coomassie brilliant blue.

In a related exemplary embodiment, the interaction between a polypeptide of interest, for instance, p300, GABP, CBP, and other cellular proteins, such as transcription factors, may be detected by co-immunoprecipitation of the polypeptide of interest with antibodies specific to the polypeptide, for instance, p300-specific antibodies. For example, in the case of p300, cellular protein extracts are incubated with purified p300-GST fusion proteins to enable protein-protein interactions. p300-specific antibodies are then added and the mixture is incubated overnight at 4° C. Immune complexes are precipitated by addition of a secondary antibody directed against the primary p300 antibodies and the precipitates are resolved by electrophoresis on denaturing polyacrylamide gels. Proteins are subsequently detected by staining with coomassie brilliant blue.

(b) Antibody Supershift Assay

In a related exemplary embodiment, DNA-protein complexes are detected by electrophoretic gel mobility shift assay (Gizard 2001, Ibid, Ausubel 1999, Ibid). Radiolabeled DNA carrying the polypeptide binding site, for instance, p300 binding site, or N-box, is mixed with a recombinant polypeptide, for instance, p300, or GABP, expressed as GST fusion protein. Subsequent resolution by electrophoresis through nondenaturing polyacrylamide gels in parallel with labeled DNA alone, reveals a shift in electrophoretic mobility only if the polypeptide is bound to DNA in the DNA/polypeptide mixture. To identify the bound polypeptide, a specific antibody is reacted to the DNA/polypeptide mixture prior to electrophoresis. Bound antibody molecules cause a further change in gel mobility, namely a supershift, and serve to identify the polypeptide bound to DNA.

(8), Identification of a DNA Consensus Binding Site

(a) PCR and DNA Sequencing

In one exemplary embodiment, DNA fragments are prepared containing potential polypeptide binding sites, either wild type or variants, flanked by DNA fragments of known nucleotide sequence. The fragments are then reacted with the polypeptide-GST fusion proteins immobilized on sepharose beads. After washing to remove adventitiously bound DNA, bound fragments are eluted by heating in presence of a detergent. The eluted fragments are amplified by the polymerase chain reaction (PCR) using primers specific for the flanking DNA sequences. The nucleotide sequence of the amplification products is then determined by any sequencing method known in the art, for instance, the dideoxy chain termination sequencing method of Sanger (Sanger 1977¹⁶¹), using as sequencing primer one of the two PCR primers. Several sequence variants of the binding site are likely to be identified. Together they can be used to establish a consensus DNA sequence for the polypeptide binding site.

(9) Detection of a Genetic Lesion

Existence of a genetic lesion can be determined by observing one or more of the following irregularities.

1. Deletion of at least one nucleotide from a disrupted gene, or gene in a disrupted pathway.

2. Addition of at least one nucleotide to a disrupted gene, or a gene in a disrupted pathway.

3. Substitution of at least one nucleotide to a disrupted gene, or gene in a disrupted pathway.

4. Irregular modification of a disrupted gene, or gene in a disrupted pathway, such as change in DNA methylation patterns.

5. Gross chromosomal rearrangement of a disrupted gene, or gene in a disrupted pathway, for instance, translocation.

6. Allelic loss of disrupted gene, or gene in a disrupted pathway.

7. Different than wild-type mRNA concentration of a disrupted gene, or gene in a disrupted pathway.

8. Irregular splicing pattern of mRNA transcript of a disrupted gene, or gene in a disrupted pathway.

9. Irregular post-transcriptional modification of an mRNA transcript other than splicing, for instance, editing, capping or polyadenylation, of a disrupted gene or gene in a disrupted pathway.

10. Different than wild-type concentration of a disrupted polypeptide, or polypeptide in a disrupted pathway.

11. Irregular post-translational modification of a disrupted polypeptide, or a polypeptide in a disrupted pathway.

Many assays are known in the art for detection of the above, or other irregularities associated with a genetic lesion. Consider the following exemplary assays. Also consider the exemplary assays discussed in the following reviews on detection of genetic lesions, Kristensen 2001¹⁶², Tawata 2000¹⁶³, Pecheniuk 2000¹⁶⁴, Cotton 1993¹⁶⁵, Prosser 1993¹⁶⁶, Abrams 1990¹⁶⁷, Forrest 1990¹⁶⁸.

(a) Sequencing

In one exemplary embodiment, a polynucleotide of interest can be sequenced using any sequencing techniques known in the art to reveal a lesion by comparing the test sequence to wild-type control, known mutant sequence, or sequences available in public databases.

An introduction to sequencing is available in Graham 2001¹⁶⁹. Exemplary sequencing protocols are available in Rapley 1996¹⁷⁰. Recent sequencing methods are available in Marziali 2001¹⁷¹, Dovichi 2001¹⁷², Huang 1999¹⁷³, Schmalzing 1999¹⁷⁴, Murray 1996¹⁷⁵, Cohen 1996¹⁷⁶; Griffin 1993¹⁷⁷. Automated sequencing methods are available in Watts 2001¹⁷⁸, MacBeath 2001¹⁷⁹, and Smith 1996¹⁸⁰. For classical sequencing methods, see Maxam 1977¹⁸¹, Sanger 1977 (Ibid).

(b) Restriction Enzyme Cleavage Patterns

In another exemplary embodiment, patterns of restriction enzyme cleavage are analyzed to reveal lesions in a polynucleotide of interest. For example, sample and control DNA are isolated, amplified, if necessary, digested with one or several restriction endonucleases, and the fragments separated by gel electrophoresis. Sequence specific ribozymes are then used to detect specific mutations by development or loss of a ribozyme cleavage site.

(c) Protection from Cleavage Agents

In another exemplary embodiment, cleavage agents, such as certain single-strand specific nucleases, hydroxylamine, osmium tetroxide or piperidine, are used to detect mismatched base pairs in nucleic acid hybrids comprised of either RNA/RNA or RNA/DNA duplexes. Wild-type and test DNA or RNA, with one or the other molecule labeled with radioactivity, are mixed under conditions permitting formation of heteroduplexes between the two species. Following hybridization, the duplexes formed are treated with an agent capable of cleaving single, but not double stranded nucleic acids. Examples include, but are not limited to S1 nuclease, piperidine, hydroxylamine, and RNase H, in the case of RNA/DNA heteroduplexes. Since mismatches between wild-type and mutant oligonucleotide result in single stranded regions, mismatch sites are susceptible to digestion. Once cleaved, the nucleic acid fragments are separated according to size by native polyacrylamide gel electrophoresis. Genetic lesion are detected by, for instance, observing different fragment sizes in test relative to wild-type DNA or RNA.

Examples of such assay in practice are available in Saleeba 1992¹⁸², Takahashi 1990¹⁸³, Cotton 1988¹⁸⁴, Myers 1985A¹⁸⁵, Myers 1985B¹⁸⁶.

(d) Mismatched Base Pairs Recognition

In another exemplary embodiment, mismatch cleavage reactions are carried out using one or more proteins capable of recognizing mismatched base pairs. The proteins are typically components of the naturally occurring DNA mismatch repair mechanism. In a preferred embodiment, the mutY enzyme derived from E. coli cleaves the adenine at a G/A mismatch (Xu 1996¹⁸⁷). The enzyme thymidine DNA glycosylase, isolated from the human cell line HeLa, cleaves the thymidine at G/T mismatches (Hsu 1994¹⁸⁸). In practice, a probe is used comprising the wild-type sequence of interest. The probe is hybridized to DNA, or cDNA corresponding to mRNA of interest. Once duplex formation has reached completion, a DNA mismatch repair enzyme is added to the reaction, and the products of the cleavage are detected by, for instance, separating reactants by denaturing polyacrylamide gel electrophoresis.

(e) Alterations in Electrophoretic Mobility

In another exemplary embodiment, variations in electrophoretic mobility are used to identify genetic lesions, by standard techniques, such as single strand conformation polymorphism (SSCP) (Miterski 2000¹⁸⁹, Jaeckel 1998¹⁹⁰, Cotton 1993, Ibid, Hayashi 1992¹⁹¹). Dilute preparations of radiolabeled single-stranded DNA fragments of test and control nucleic acids, separately, are denatured by heat and permitted to renature slowly. Upon renaturation, single stranded nucleic acids in the dilute solutions form secondary structures. Each molecule forms internal base paired regions depending on each molecule sequence. Consequently, wild-type and mutant sequences, otherwise identical except for regions of mutation, form different secondary structures. Each preparation is separated in adjacent lanes by electrophoresis through native polyacrylamide gels while preserving the secondary structure formed during renaturation. Alterations in electrophoretic mobility reveal differences between wild-type and mutant oligonucleotides as small as single nucleotide differences. Following electrophoresis, the radiolabeled nucleic acids are detected by autoradiography or by phosphorimager analysis. A variation of this assay employs RNA rather than DNA.

In a related exemplary embodiment, wild-type and mutant DNA molecules are separated by electrophoresis through polyacrylamide gels containing a gradient of denaturant. The method, termed “denaturing gradient gel electrophoresis,” (DGGE) (Myers 1985B, Ibid) is commonly used to detect differences between similar oligonucleotides. Prior to analysis, test DNA is often modified by addition of up to 40 base pairs of GC rich DNA through PCR. The relatively stable region, termed “GC clamp,” ensures only partial denaturation. A variation of the assay employs a temperature rather than chemical gradient of denaturant.

(f) Selective Oligonucleotide Hybridization

In another embodiment, selective hybridization involves the use of synthetic oligonucleotide primers prepared to carry a known mutation in a central position. Primers are then mixed with test DNA under conditions permitting hybridization for perfectly matched molecules (Lipshutz 1995¹⁹², Guo 1994¹⁹³, Saiki 1989¹⁹⁴). The allele specific oligonucleotide (ASO) hybridization method can be used to test a single mutation per reaction mixture, or many different mutations if the ASO is first immobilized on a suitable membrane. The technique, termed “dot blotting,” permits rapid screening of many mutations when nonimmobilized DNA is first radiolabeled to permit visualization of the immobilized hybrids.

(g) Allele Specific Amplification

Under certain conditions, polymerase extension occurs only if there is a perfect match between primer and the 3′ terminus of the 5′, left-most or upstream region of a sequence of interest. Therefore, in another embodiment, allele specific amplification, a selective PCR amplification based assay, a synthetic oligonucleotide primer is prepared carrying a mutation at the center, or extreme 3′ end of the primer, such that mismatch between primer and test DNA prevents, or reduces efficiency of the polymerase extension during amplification (Efremov 1991¹⁹⁵, Gibbs 1989¹⁹⁶). A mutation in the test DNA is detected by a change in amplification product concentration relative to controls, or, in special cases, by the presence or absence of amplification products.

A variation of the assay introduces a novel restriction endonuclease recognition site in the expected mutation region to permit detection by restriction endonuclease cleavage of the amplification products (see also above).

(h) Protein Truncation Test

Another embodiment uses the protein truncation test (PTT). If a mutation introduces a premature translation stop site, PTT offers an effective detection assay Geisler 2001¹⁹⁷, Moore 2000¹⁹⁸, van der Luijt 1994¹⁹⁹, Roest 1993²⁰⁰) In this assay, RNA is isolated from sample cells or tissue and converted to cDNA by reverse transcriptase. The sequence of interest is amplified by the PCR, and the products are subjected to another round of amplification with a primer carrying a promoter for RNA polymerase, a sequence for translation initiation. The products of the second round of PCR are subjected to transcription and translation in vitro. Electrophoresis of the expressed polypeptides through sodium dodecyl sulfate (SDS) containing polyacrylamide gels reveals the presence of truncated species arising from the presence of premature translation stop sites. In a variation of this assay, if the sequence of interest is contained within a single exon, DNA rather than cDNA can be used as PCR amplification template.

(i) General Comments

Any tissue or cell type expressing a sequence of interest may be used in the described assays. For instance, bodily fluids, such as blood obtained by venipuncture or saliva, or non-fluid samples, such as hair, or skin, may be used. Samples of fetal polynucleotides collected from maternal blood, amniocytes derived from amniocentesis, or chorionic villi obtained for prenatal testing, can also be used.

Pre-packaged diagnostic kits containing one or more nucleic acid probes, primer set, and antibody reagent may be useful in performing the assays. Such kits are designed to provide an easy to use instrument especially suitable for use in the clinic.

The assays may also be applied in situ directly on the tissue to be tested, fixed, or frozen. Typically, such tissue is obtained in biopsies, or surgical procedures. In situ analysis precludes the need for nucleic acid purification.

While the exemplary assays described so far primarily permit the analysis of one nucleic acid sequence of interest, they may be also used to generate a profile of multiple sequences of interest. The profile may be generated, for example, by employing Northern blot analysis, a differential display procedure, or reverse transcriptase-PCR (RT-PCR).

In addition to nucleic acid assays, antibodies directed against a mutated polynucleotide, or polypeptide product of a mutated polynucleotide may be used in various assays (see below).

(10) Assaying Methylation Status of DNA

(a) Sodium Bisulfite Method

In one exemplary embodiment, the methylation status of DNA sequences can be determined by first isolating cellular DNA, and then converting unmethylated cytosines into uracil by treatment with sodium bisulfite, leaving methylated cytosines unchanged. Following treatment, the bisulfite is removed, and the chemically treated DNA is used as a template for PCR. Two parallel PCR reactions are performed for each DNA sample, one using primers specific for the DNA prior to bisulfite treatment, and one using primers for the chemically modified DNA. The amplification products are resolved on native polyacrylamide gels and visualized by staining with ethidium bromide followed by UV illumination. Amplification products detected from the sodium bisulfite treated samples indicate methylation of the original sample.

Specifically, this assay can be used to asses the methylation status of DNA binding sites of a polypeptide of interest, such as GABP, p300, CBP, etc.

(11) Assaying Protein Phosphorylation

(a) Western Blot with Antiphosphotyrosine

In one exemplary embodiment, protein phosphorylation is measured using anti-phosphotyrosine antibodies (for instance, antibodies available from Santa Cruz Biotechnology, catalog numbers sc-508 or sc-7020). Boiling in detergent-containing buffer lyses cultured cells. Proteins contained in the cell lysate are separated by electrophoresis through SDS polyacrylamide gels followed by transfer to a nylon membrane by electrophoresis, a process termed electroblotting (Burnett 1981²⁰¹). Prior to incubation with antibody, the membrane is incubated with blocking buffer containing the nonionic detergent Tween 20 and nonfat dry milk as a source of protein to later block adventitious binding of specific antibodies to the nylon membrane. The immobilized proteins are then reacted with anti-phosphotyrosine antibodies and visualized after reaction with a secondary antibody conjugated to horse radish peroxidase. Exposure to hydrogen peroxide in presence of the chromogenic indicator diaminobenzidine produces visible bands where secondary antibodies are bound, thereby enabling their localization.

A variation of this assay can be performed with antibodies directed against phosphothreonine (for instance, those available from Santa Cruz Biotechnology, catalog number sc-5267) or a host of phosphorylated molecules. Sources of available phosphoprotein specific antibodies include, but are not limited to, Santa Cruz Biotechnology of Santa Cruz, Calif., Calbiochem of San Diego, Calif., and Chemicon International, Inc. of Temecula, Calif.

The protein phosphorylation detection assays may be employed before and/or after treatment with an agent of interest to detect changes in phosphorylation status of a polypeptide, or group of polypeptides. Moreover, detection of changes in phosphorylation status of polypeptides of interest may be used to monitor efficacy of a therapeutic treatment or progression of a chronic disease.

(b) Immunoprecipitation

In one complementary embodiment, the relative levels of phosphorylated and nonphosphorylated forms of any particular protein may be measured. The levels of the phosphorylated forms are measured as described above. Nonphosphorylated proteins are measured by first immunoprecipitating all forms of the protein of interest with a specific antibody directed toward that protein. Western blotting as described then analyzes the immune complexes. Comparison of the levels of total protein of interest to those of the phosphorylated forms provides some insight into the relative levels of each form of the polypeptide of interest.

(12) Assaying Gene Activation and Suppression

(a) Co-Transfection with Report Gene to Identify Transactivators

In one exemplary embodiment, interactions between regulatory proteins and a DNA sequence of interest can be revealed through co-transfection of two recombinant vectors. The first vector carries a full-length cDNA for the regulatory factor driven by a promoter known to be active in the transfected cells. The second recombinant vector carries a reporter gene driven by the DNA sequence of interest. Examples of suitable reporter genes include chloramphenicol acetyltransferase (CAT), luciferase, or β-galactosidase (Virts 2001²⁰²). Detection of reporter gene expression by methods known in the art (see examples below) indicates transactivation of the DNA sequence of interest by the regulatory factor.

Transfection of appropriate recombinant vectors can be mediated either with calcium phosphate (Chen 1988²⁰³) or DEAE-dextran (Lopata 1984²⁰⁴); In one exemplary embodiment, exponentially growing cells are exposed to precipitated DNA. A DNA solution, prepared in 0.25M CaCl₂ is added to an equal volume of HEPES buffered saline and incubated briefly at room temperature. The mixture is then placed over cells and incubated overnight to permit DNA adsorption and absorption into the cells. The next day the cells are washed and cultured in complete growth medium.

In a related exemplary embodiment, calcium chloride precipitation is replaced with DEAE-dextran as a carrier for the DNA to be transfected. Growth medium is made 2.5% with respect to fetal bovine serum (FBS) and 10 μM with respect to chloroquine. The medium is prewarmed, and DNA is added prior to addition of DEAE-dextran. The mixture is then added to exponentially growing cells, and incubated for 4 hours to allow DNA adsorption. The transfection medium is replaced by a 10% solution of DMSO causing the DNA to enter the cells. The cells are incubated for 2-10 hours. The DMSO solution is then replaced by growth medium, and the cells are incubated until assayed for exogenous gene expression.

CAT

Detection of CAT gene expression is achieved by mixing lysates of the cells in which the reporter gene has been co-transfected along with a recombinant vector carrying the putative activating factor with ¹⁴C-labeled chloramphenicol (Gorman 1982²⁰⁵). Acetylated and unacetylated forms of the compound, the latter resulting from enzymatic degradation of the substrate by expressed CAT, are separated by thin layer chromatography and visualized by autoradiography. Measurements of each radiolabeled species are attained by densitometric analysis of the autoradiograph, or by direct phosphorimager analysis of the chromatograph.

Luciferase

Detection of expressed luciferase is achieved by exposure of transfected cell lysates to the luciferase substrate luciferin in presence of ATP, magnesium, and molecular oxygen (Luo 2001²⁰⁶) The presence of luciferase results in transient release of light detected by luminometer.

β-Galactosidase

Detection of β-galactosidase gene expression is achieved by mixing cell lysates with a chromogenic substrate for the enzyme, such as o-nitrophenyl-β-D-galactopyranoside (ONPG), or a chemiluminescent substrate containing 1,2 dioxetane. Products of the catalytic degradation of the chromogenic substrate are easily visualized, or alternatively, quantified by spectrophotometry, while the products of the chemiluminescent substrate are detected by luminometer. The latter assay is especially sensitive and can detect minute levels, or minute changes in levels of β-galactosidase reporter gene expression.

These assays were applied to demonstrate binding of GABP to the promoter regions of a number of genes including the retinoblastoma gene (Sowa 1997²⁰⁷), CD18 (Rosmarin 1998, ibid), cytochrome C oxidase Vb (Sucharov 1995²⁰⁸) and the prolactin gene (Ouyang 1996²⁰⁹).

(b) Co-Transfection with Reporter Gene to Identify Trans-Acting Repressors

These assays can be applied to assess trans-acting factors that potentially repress rather than stimulate reporter gene expression. In this embodiment, putative repression factors are expressed from a recombinant vector in cells that carry a reporter gene driven by a constitutively active promoter that may interact with the repression factor. The assays described above are applied to determine whether expression of the repression factor reduces reporter gene activity.

(13) Assaying Gene Expression Levels

(a) Northern Blot Analyses

In one exemplary embodiment, the relative expression levels of a gene of interest are measured by Northern blot analysis (Ausubel 1999, Ibid). RNA is isolated from untreated cells and cells after treatment with an agent expected to modulate gene expression. The RNA is separated by electrophoresis through a denaturing agarose gel, typically incorporating the denaturant formaldehyde, and transferred to a nylon membrane. Immobilized RNA is hybridized to a radiolabeled DNA probe representing the gene of interest. Bound radiolabel is visualized by autoradiography. Scanning the resulting autoradiograph with a densitometer and integrating the area under the traces can quantify levels of bound radiolabel. Alternatively, incorporated radiolabel can be quantified by phosphorimager analysis of the blot itself.

(b) RT-PCR

In a related embodiment, RNA is isolated from similarly treated cells. The RNA is then subjected to reverse transcription (RT) and amplification by the polymerase chain reaction (PCR) in the presence of radiolabeled deoxynucleotides. The amplification products are resolved by gel electrophoresis and visualized by autoradiography. Scanning the resulting autoradiograph with a densitometer and integrating the area under the traces can quantify levels of incorporated radiolabel. Alternatively, incorporated radiolabel can be quantified by phosphorimager analysis of the electrophoretic gel.

(14) Assaying Viral Replication

(a) Viral Titer

In one exemplary embodiment, viral replication is measured by titration of infectious particles on cultured host cells. Virus replication is permitted in host cells, with or without chemical treatment, or with or without co-expression of a regulatory gene, for a measured period of time. The cells are lysed by exposure to a hypotonic solution, and the lysates are subjected to a series of dilutions in isotonic buffer. Several concentrations of cell lysate are separately plated onto cultured host cells. The culture cells are incubated until the cytopathic effects (CPE) are evident. The cultured cells are then fixed and stained with a contrast enhancing dye, such as crystal violet, to facilitate identification of viral plaques. Several culture plates are counted, and the number of plaques multiplied by the appropriate dilution factor, representing the dilution from the original cell lysate. The result reveals the viral titer of the original cell lysate.

(b) In Situ PCR

In a related exemplary embodiment, a latent, low copy number virus can be detected with the polymerase chain reaction in situ (Staskus 1994²¹⁰). Cells grown either in suspension culture or on a solid substrate are fixed and permeabilized. PCR reaction components, including synthetic primers complementary to the gene of interest, Taq polymerase, deoxyribonucleotides, are then added to the cells and subjected to thermal cycling typical of PCR. The amplification products, retained in each cell, are detected by in situ hybridization with appropriately labeled DNA probes. An exemplary detection method involves hybridization with radiolabeled probes followed by autoradiography. Similarly, hybridization probes may be nonradioactively labeled by including digoxygenin-11-dUTP into the PCR reaction. Incorporated label is detected either enzymatically or chemically.

(15) Assaying Cell Morphology and Function

(a) Light Microscopy

In one exemplary embodiment, the morphology of cells is ascertained by microscopic examination. Statin trypan blue can distinguish between living and dead cells (Schuurhuis 2001²¹¹). Living cells, with intact cellular membranes, exclude trypan blue while dead cells, with leaky, or perforated outer membranes, permit trypan blue to enter the cytoplasm. Following treatment, examination by phase contrast microscopy reveals the proportion of dead vs. living cells. Similarly, cellular morphology can be ascertained by examination with phase contrast microscopy, with or without prior staining, with, for example, crystal violet, to enhance contrast. Such examination reveals morphologies common to known cell types, and concomitantly reveals irregularities present in the cell population under examination.

(b) Functional Assessment by Immunocytochemistry

In a related exemplary embodiment, the functional status of a given cell population may be determined by treatment with specific antibodies. Cells are dehydrated and fixed with a series of methanol washes using increasing concentrations of methanol. Once fixed, the cells are exposed to cell-type specific antibodies. Examples of suitable antibodies include, but are not limited to, anti-filaggrin for epidermal cells, anti-CD4 for T cells, thymocytes and monocytes, and anti-macrosialin for macrophages. After incubation with differentiation-specific marker antibodies, fluorescently labeled secondary antibodies specific for the first antibody are added. Bound secondary antibodies are visualized by illumination with light of appropriate wavelength to excite the bound fluorochrome followed by microscopic examination. The use of different antibodies, each conjugated to a different fluorochrome, permits the identification of multiple differentiation-specific antigens simultaneously in the same population of cells.

(16) Assaying Cellular Oxidation Stress

(a) Cellular Indicators

In one exemplary embodiment, oxidation stress within a population of cells can be measured by assaying the activity levels of certain indicators such as lipid hydroperoxides (Weyers 2001²¹²). Cell lysates are prepared and mixed with the substrate 1-napthyldiphenylphosphiine (NDPP). Any resulting oxidized form of the substrate, ONDPP, can be quantified by high performance liquid chromatography (HPLC). ONDPP concentration provides an indirect measure of the oxidation capacity of the cell lysate.

(b) H2DCFDA as Indicator

In another exemplary embodiment, the production of cellular reactive oxygen species can be detected by mixing cell lysates with 2′,7′-dichlorodihydrofleuoescein diacetate (H2DCFDA) (Brubacher 2001²¹³). In the presence of cellular esterases, H2DCFDA is deacetlyated to produce 2′,7′-dichlorodihydrofleuoescein (H2DCF), an oxidant-sensitive indicator. Increased cellular oxidation excites the fluorogenic indicator. Using H2DCF directly can attain increased sensitivity, but caution must be exercised by one skilled in the art to ensure that none of the experimental buffers contain contaminants, such as metals, which may lead to spontaneous fluorescence.

d) Optimization Protocols

Once a single constructive or disruptive agent (polynucleotide, polypeptide, small molecule, etc.) is identified in the manner described above, variant agents can be formulated that improve upon the original agent.

The expression “variant agents . . . that improve upon the original agent” is understood to include, but not be limited to, agents that increase therapeutic efficacy, increase prophylactic potential, increase, or decrease stability in vivo or in storage, or increase the number, or variety of post-translational modifications in vivo, including, but not limited to, phosphorylation, acetylation and glycosylation, relative to the original agent.

Variant agents are not limited to those produced in the laboratory. They may include naturally occurring variants. For example, variants with increased stability, due to alterations in ubiquitination or modifications of other target sites conferring resistance to proteolytic degradation.

e) Treatment Protocols

(1) Introduction

According to the present invention, a polypeptide has a constructive effect if it attenuates microcompetition with a foreign polynucleotide or attenuates at least one effect of microcompetition with a foreign polynucleotide, or one effect of another foreign polynucleotide-type disruption. For example, a constructive polypeptide can reduce copy number of the foreign polynucleotide, stimulate expression of a GABP regulated gene, increase bioactivity of a GABP regulated protein, through, for instance, GABP phosphorylation and/or increase bioavailability of a GABP regulated protein, through, for instance, a reduction in copy number of microcompeting foreign polynucleotides which bind GABP. A constructive polypeptide can also, for example, inhibit expression of a microcompetition-suppressed gene, such as, tissue factor, androgen receptor, and/or inhibit replication of a p300/cbp virus (see more examples below).

Agents of the present invention are designed to address and ameliorate symptoms of chronic diseases, specifically, diseases resulting from microcompetition between a foreign polynucleotide and cellular genes. For instance, introduction of an oligonucleotide agent into a cell may disrupt this microcompetition and restore normal regulation and expression of a microcompeted gene. Agents directed against a foreign polynucleotide may reduce binding or cellular transcription factors to the foreign polynucleotide by, for instance, reducing the copy number of the foreign polynucleotide, or its affinity to the transcription factor, resulting in increased microavailability of the factors towards normal levels. Alternatively, binding of the transcription factors to cellular genes can be stimulated. In yet another exemplary embodiment, insufficient, or excessive expression of a cellular gene in a subject can be modified by administration of nucleic acids or polypeptides to the subject that return the concentration of a cellular polypeptide of interest towards normal levels.

The following section describes standard protocols for determining effective dose, and for agent formulation for use. Additional standard protocols and background information are available in books, such as In vitro Toxicity Testing Protocols (Methods in Molecular Medicine, 43), edited by Sheila O'Hare and C K Atterwill, Humana Press, 1995; Current Protocols in Pharmacology, edited by: S J Enna, Michael Williams, John W Ferkany, Terry Kenakin, Roger D Porsolt, James P Sullivan; Current Protocols in Toxicology, edited by: Mahin Maines (Editor-in-Chief), Lucio G Costa, Donald J Reed, Shigeru Sassa, I Glenn Sipes; Remington: The Science and Practice of Pharmacy, edited by Alfonso R Gennaro, 20^{th} edition, Lippincott, Williams & Wilkins Publishers, 2000; Pharmaceutical Dosage Forms and Drug Delivery Systems, by Howard C Ansel, Loyd V Allen, Nicholas G Popovich, 7^{th} edition, Lippincott Williams & Wilkins Publishers, 1999; Pharmaceutical Calculations, by Mitchell J Stoklosa, Howard C Ansel, 10^{th} edition, Lippincott, Williams & Wilkins Publishers, 1996; Applied Biopharmaceutics and Pharmacokinetics, by Leon Shargel, Andrew B C Yu, 4t edition, McGraw-Hill Professional Publishing, 1999; Oral Drug Absorption: Prediction and Assessment (Drugs and the Pharmaceutical Sciences, Vol 106), edited by Jennifer B Dressman, Hans Lennernas, Marcel Dekker, 2000; Goodman & Gilman's The Pharmacological Basis of Therapeutics, edited by Joel G Hardman, Lee E Limbird, 10^{th} edition, McGraw-Hill Professional Publishing, 2001. See also above referenced.

(2) Effective Dose

Compounds can be administered to a subject, at a therapeutically effective dose, to treat, ameliorate, or prevent a chronic disease. Careful monitoring of patient status, using either systemic means, standard clinical laboratory assays or assays specifically designed to monitor the bioactivity of a foreign polynucleotide, is necessary to establish the therapeutic dose and monitor its effectiveness.

Prior to patient administration, techniques standard in the art are used with any agent described herein to determine the LD₅₀ and ED₅₀ (lethal dose which kills one half the treated population, and effective dose in one half the population, respectively) either in cultured cells or laboratory animals. The ratio LD₅₀/D₅₀ represents the therapeutic index, which indicates the ratio between toxic and therapeutic effects. Compounds with a relatively large index are preferred. These values are also used to determine the initial therapeutic dose. While unwanted side effects are sometimes unavoidable, they may be minimized by delivery of the therapeutic agent directly to target cells or tissues, thereby avoiding systemic exposure.

Those skilled in the art recognize that animal or cell culture models are imperfect predictors of the efficacy of any treatment in humans. Factors affecting efficacy include route of administration, achievable serum concentration and formulation of the therapeutic agent (i.e. in pill or injectable forms, administered orally or intramuscularly, with accompanying carrier, formulation of an agent adducted with a specific antibody and injected directly into the target tissue, etc.). Regardless of the method of delivery or formulation of the therapeutic agent, it is important to monitor plasma levels using a suitable technique, such as atomic absorption spectroscopy, enzyme linked immunosorbant assay (ELISA), or high performance liquid chromatography (HPLC) among others.

(3) Formulation for Use

Those skilled in the art recognize a host of standard formulations for the agents described in this invention. Any suitable formulation may be prepared for delivery of the agent by injection, inhalation, transdermal diffusion, or insufflation. In every case, the formulation must be appropriate for the means and route of administration.

Oligonucleotide agents, e.g. antisense oligonucleotides or recombinant expression vectors, may be formulated for localized or systemic administration. Systemic administration may be achieved by injection in a physiologically isotonic buffer including Ringer's or Hank's solution, among others. Alternatively, the agent may be given orally by delivery in a tablet, capsule, or liquid syrup. Those skilled in the art recognize pharmaceutical binding agents and carriers, which protect the agent from degradation in the digestive system and facilitate uptake. Similarly, coatings for the tablet or capsule may be used to ease ingestion thereby encouraging patient compliance. If delivered in liquid suspension, additives may be included which keep the agent suspended, such as sorbitol syrup and the emulsifying agent lecithin, among others, lipophilic additives may be included, such as oily esters, or preservatives may be used to increase shelf life of the agent. Patient compliance may be further enhanced by the addition of flavors, coloring agents, or sweeteners. In a related embodiment, the agent may be provided in lyophilized form for reconstitution by the patient or his or her caregiver.

The agents described herein may also be delivered via buccal absorption in lozenge form or by inhalation via nasal aerosol. In the latter mode of administration, any of several propellants, including, but not limited to, trichlorofluoromethane and carbon dioxide, or delivery methods, including but not limited to a nebulser, can be employed. Similarly, compounds may be included in the formulation, which facilitate transepithelial uptake of the agent. These include, among others, bile salts and detergents. Alternatively, the agents of this invention may be formulated for delivery by rectal suppository or retention enema. Those skilled in the art recognize suitable methods for delivery of controlled doses.

In related embodiments, the agents may be formulated for depot administration, such as by implantation, via regulated pumps, either implanted or worn extracorporally or by intramuscular injection. In these instances, the agent may be formulated with hydrophobic materials, such as an emulsification in pharmaceutically permissible oil, bound to ion exchange resins or as a sparingly soluble salt.

In every case, therapeutic agents destined for administration outside of a clinical setting may be packaged in any suitable way that assures patient compliance with regard to dose and frequency of administration.

Administration of the agents included in this invention in a clinical situation may be achieved by a number of means including injection. This method of systemic administration may achieve cell-type specific targeting by using a nucleic acid agent, described herein, modified by addition of a polypeptide that binds to receptors on the target cell. Additional specificity may be derived from the use of recombinant expression vectors that carry cell- or tissue-type specific promoters or other regulatory elements. In contrast to systemic injection, more specific delivery may be achieved by means of a catheter, by stereotactic injection, by electorporation or by transdermal electrophoresis. Many suitable delivery techniques are well known in the art.

In an alternative embodiment, the therapeutic agent may be administered by infection with a recombinant virus carrying the agent. Similarly, cells may be engineered ex vivo that express the agent. Those cells may themselves become the pharmaceutical agent for implantation into the site of interest in the patient.

f) Diagnosis Protocols

Diagnosis may be achieved by a number of methods, well known in the art, using as reagents sequences of a foreign polynucleotide, disrupted gene or polypeptide, or a gene or polypeptide in a disruptive or disrupted pathway, or antibodies directed against such polynucleotides or polypeptides. Those reagents may be used to detect and quantify the copy number, level of expression or persistence of expression products of a foreign polynucleotide, disrupted gene or gene susceptible to microcompetition with a foreign polynucleotide.

Diagnostic methods may employ any suitable technique well known in the art. These include, but are not limited to, commercially available diagnostic kits, which are specific for one or more foreign polynucleotides, a specific disrupted gene, a disrupted polypeptide, a gene or polypeptide in a disruptive or disrupted pathway, or an antibody against such polynucleotides or polypeptides. Well-known advantages of commercial kits include convenience and reproducibility due to manufacturing standardization, quality control, and validation procedures.

(1) Detection and Quantification of Polynucleotides

In one exemplary embodiment, nucleic acids, DNA or RNA, are isolated from a cell or tissue of interest using procedures well known in the art. Once isolated, the presence of a foreign polynucleotide may be ascertained by any of a number of procedures including, but not limited to, Southern blot hybridization, dot blotting, and the PCR, among others. Mutations in those polynucleotides may be detected by single strand conformation analysis, allele specific oligonucleotide hybridization, and related and complementary techniques. Alternatively, nucleic acid hybridization with appropriately labeled probes may be performed in situ on isolated cells or tissues removed from the patient. Suitable techniques are described, for example, Sambrook 2001 (ibid), incorporated herein in its entirety by reference. Control cells and tissues are compared in parallel to validate any positive findings in clinical samples.

If the nucleic acid molecules specific to foreign polynucleotides or disrupted genes, or genes in disrupted or disruptive pathways are in low concentration, preferred diagnostic methods employ some means of amplification. Examples of suitable procedures include the PCR, ligase chain reaction, or any of a number of other suitable methods well known in the art.

In one exemplary embodiment of a diagnostic technique employing nucleic acid hybridization, RNA from the cell of interest is isolated and converted to cDNA (using the enzyme reverse transcriptase of avian or murine origin). Once cDNA is prepared, it is amplified by the PCR, or a similar method, using a sequence specific oligonucleotide primer of 20-30 nucleotides in length. Incorporation of radiolabeled nucleotides during amplification facilitates detection following electrophoresis through native polyacrylamide gels by autoradiography or phosphorimager analysis. If sufficient amplification products are attained, they may be visualized by staining of the electrophoretic gel by ethidium bromide or a similar compound well known in the art.

(2) Detection and Quantification of Polypeptides

Antibodies directed against foreign polypeptides, disrupted polypeptides, or polypeptides in disrupted or disruptive pathways, may also be used for the diagnosis of chronic disease. Diagnostic protocols may be employed to detect variations in the expression levels of polypeptides or RNA transcripts. Similarly, they may be used to detect structural variation including nucleic acid mutations and changes in the sequence of encoded polypeptides. The latter may be detected by changes in electrophoretic mobility, indicative of altered charge, or by changes in immunoreactivity, indicative of alterations in antigenic determinants.

For diagnositic purposes, protein may be isolated from the cells or tissues of interest using any of many techniques well known in the art. Exemplary protocols are described in Molecular Cloning: A Laboratory Manual, 3rd Ed (Third Edition), by Joe Sambrook, Peter MacCallum and David Russell (Cold Spring Harbor Laboratory Press 2001), incorporated herein by reference in its entirety.

In a preferred embodiment, detection of a foreign polypeptide molecule, or a cellular disrupted polypeptide molecule, or a polypeptide in a disruptive or disrupted pathway is achieved with immunological methods, including immunoaffinity chromatography, radial immunoassays, radioimmunoassay, enzyme linked immunsorbant assay, etc. These techniques, quantitative and qualitative, all well known in the art, exploit the interaction between specific antibodies and antigenic determinants on the target molecule. In each assay, polyclonal or monoclonal antibodies, or fragments thereof, may be used as appropriate.

Immunological assays may be employed to analyze histological preparations. In a preferred embodiment, tissue or cells of interest are treated with a fluorescently labeled specific antibody or an unlabeled antibody followed by reaction with a secondary fluorescently labeled antibody. Following incubation for sufficient time and under appropriate conditions for antibody-antigen interaction, the label may be visualized microscopically, in the case of either tissues or cells, or by flow cytometry, in the case of individual cells. These techniques are particularly suitable for antigens expressed on the cell surface. If they are not on the cell surface, the cells or tissue to be analyzed must be treated to become permeable to the diagnostic antibodies. In addition to the detection of antigens on the material studied, the distribution of that antibody will become evident upon microscopic examination. All immunological assays involve the incubation of a biological sample, cells or tissue, with an appropriately specific antibody or antibodies. These and other suitable diagnostic methods are familiar to those skilled in the art.

In an alternative embodiment, immunological techniques may be employed which involve either immobilized antibodies or immobilizing the cells to be analyzed on, for example, synthetic beads or the surface of a plastic dish, typically a microtiter plate (see above).

Immobilization of antibodies or cells to be analyzed is achieved through the use of any of several substrates well known in the art including, but not limited to, glass, dextran, nylon, cellulose, and polypropylene, among others. The actual shape or configuration of the substrate may vary to suite the desired assay. For example, polystyrene may be formed into tissue culture or microtitre plates, dextran may be formed into beads suitable for column chromatography, or polyacrylamide may be coated onto the inner surface of a glass test tube or bottle. These and related carriers and configurations are well known and can be tested for utility by those skilled in the art.

Detection of bound antibodies is achieved by labeling, either directly or indirectly, by a secondary antibody specific for the first. The label may be either a chromophore, which responds to excitation by a specific wavelength of light, thereby producing fluorescence, or it may be an enzyme, which reacts with a chromogenic substrate to produce detectable reaction products. Common florescent labels include fluorescineisothiocyanate (FITC), rhodamine and trans-1-bromo-2,5-bis-(3-hydroxycarbonyl-4-hydroxy)styrylbenzene (BSB), among others. Enzymes commonly conjugated with antibodies include, but are not limited to, alkaline phosphatase, horseradish peroxidase, and β-galactosidase. Other alternatives are available and well known in the art.

In a related embodiment, the antibody is labeled with a fluorescent metal, for example ¹⁵²Eu, which can be attached directly to the primary or secondary antibody in an immunoassay. Alternatively, the antibody may be labeled with a chemiluminescent compound, such as luminol, isoluminol or imidazole or a bioluminiscent compount, such as luciferin or aequorin. Subsequent reaction with the appropriate substrate for the labeling compound produces light, which is detectable visually or by fluorimetry.

(3) Imaging of Diseased Tissues

Under suitable circumstances, foreign polypeptides, polypeptides expressed from disrupted genes, or from genes in a disruptive or disrupted pathway, may be detected on the surface of affected cells or tissues. In these instances the level and pattern of expression may be visualized and used to both diagnose disease and to guide and gauge therapy. For example, in atherosclerosis, such disrupted polypeptides may include, but are not limited to CD18 or tissue factor (see more details in examples below).

Under these circumstances, antibodies, monoclonal or polyclonal, which specifically interact with proteins expressed on the cell surface, may be used for the diagnosis of chronic disease and for monitoring treatment efficacy. In this embodiment, an appropriate antibody or antibody fragment is labeled with a radioactive, fluorescent, or other suitable tag prior to reaction with the biomaterial to be assayed. Conditions for reaction and visualization are well known in the art and permit analyses to be carried out in vitro as well as in situ. In a preferred embodiment, antibody fragments are used for in situ or in vitro assays because their smaller size leads to more rapid accumulation in the tissue of interest and more rapid clearing from that tissue following the assay. A number of suitable and appropriate labels may be used for the assays in this invention that are well known in the art.

g) Clinical Trials

Another aspect of current invention involves monitoring the effect of a compound on a treated subject in a clinical trial. In such a trial, the copy number of a foreign polynucleotide, its affinity to cellular transcription factors, the expression or bioactivity of a disrupted gene or polypeptide, or expression or bioactivity of a gene or polypeptide in a disrupted or disruptive pathway, may be used as an indicator of the compound effect on a disease state.

For example, to study the effect of a test compound in a clinical trial, blood may be collected from a subject before, and at different times following treatment with such a compound. The copy number of a foreign polynucleotide may be assayed in monocytes as described above, or the levels of expression of a disrupted gene, such as tissue factor, may be assayed by, for instance, Northern blot analysis, or RT-PCR, as described in this application, or by measuring the concentration of the protein by one of the methods described above. In this way, the copy number, or expression profile of a gene of interest or its mRNA, may serve a surrogate or direct biomarker of treatment efficacy. Accordingly, the response may be determined prior to, and at various times following compound administration. The effects of any therapeutic agent of this invention may be similarly studied if, prior to the study, a suitable surrogate or direct biomarker of efficacy, which is readily assayable, was identified.

B. Examples

The current view holds that, in vivo, viral proteins are the sole mediators of viral effects on the host cell. Such proteins include, for example, the papilomavirus type 16 E6 and E7 oncoproteins, SV40 large T antigen, Epstein-Barr virus BRLF1 protein, and adenovirus E1A. The possibility that presence of viral DNA in the host cell can directly impact cell function, independent of viral protein, is typically ignored. The viral “protein-dependent” view is so ingrained in current research that in many cases, when a “protein-independent” effect on cellular gene expression, or other cell functions, presents itself in the laboratory, the effect is ignored. As a result, the significance of such effect, and specifically, its relation to disease is overlooked. Note that the effect of viral DNA on the cellular genome in cases of viral DNA integration which may result in mutations, deletions or methylation of host cell DNA, cannot be considered “protein-independent” since it is mediated by viral proteins, such as, HIV-1 IN protein, or retrovirus integrase. The following examples illustrate the invention. More examples can be found in PCT patent application PCT/US01/05314, and U.S. patent application Ser. Nos. 10/223,050 and 10/209,206 incorporated herein in its entirety by reference.

The present invention starts from the discovery that microcompetition is involved in a variety of human diseases. It is only by looking through the lens of the present invention that a discernable pattern of disease progression and symptomology is understood. From this understanding, the inventor was able to develop new assays, screening regiments and treatments.

The full citation for each reference is provided at the end of the detailed disclosure and is cited in an abbreviated fashion within the text to make the disclosure more readable.

1. Preface

This examples section of the specifications presents a theory. The theory identifies the origin of many-chronic diseases, such as atherosclerosis, stroke, cancer, obesity, diabetes, multiple sclerosis, lupus, thyroiditis, osteoarthritis, rheumatoid arthritis, and alopecia.

Take a set of empirical papers. Present all observations reported in these papers as dots on a plain background. FIG. 1 illustrates a collection of such dots.

Dots represent observations, or facts. A collection of lines, connecting a set of dots, represents a theory. A theory is a picture anchored in a set of dots. FIG. 2 (see p. 128) presents a theory anchored in the dots illustrated in FIG. 1.

Theory as a picture is an old idea. In Greek, the root word thea means “to see.” Theoria, a related word, means spectacle, or viewing from a distance, as a whole. Distance is important. Being too close to any one dot is distractive. Only from a distance, one can grasp the entire picture. A similar effect is illustrated by the artist's practice of stepping back from the canvas when examining the painting.

Empirical studies produce dots. Theoretical studies produce lines. A line is a relation between dots. A theory relates seemingly unrelated observations. According to Webster's dictionary, a theory is “the analysis of a set of facts in their relation to one another.” An observation is a fact. The set of lines connecting facts is a theory.

Every line connects two dots. However, a line by itself is a collection of an infinite number of other dots. Each such new dot is a prediction. The unfilled dot FIG. 2 illustrates a prediction.

The unfilled dot also clarifies a common confusion between theory and hypothesis. The confusion is so ingrained, that according to Webster's dictionary, theory also means speculation,” or “unproved hypothesis.” The picture is a theory. A new dot at a certain spot on a certain line is a hypothesis. No theory, no hypothesis.

In the past, the theoretical method was used in biology to produce major discoveries. In their single page famous paper, Watson and Crick include one paragraph describing their scientific method.

“The previously published X-ray data on deoxyribose nucleic acid are insufficient for a rigorous test of our structure. So far as we can tell, it is roughly compatible with the experimental data, but it must be regarded as unproved until it has been checked against more exact results. Some of these are given in the following communications. We were not aware of the details of the results presented there when we devised our structure, which rests mainly though not entirely on published experimental data and stereochemical arguments.” (Watson 1953²¹⁴, underline added).

Friedman and Friedland, the authors of the book “Medicine's 10 greatest discoveries,” provide the following comments on the approach used by Watson and Crick (Friedman 1998²¹⁵, underline added):

“Perhaps never before in the history of science was such a great scientific discovery achieved with so much theoretical conversation and so little experimental activity” (p. 214).

“Never before has such a discovery been made by the simple combination of blackboard scrawling, absorption of the experimental work of others, perusal of other scientist' publications, and manipulation of plastic balls, wires and metal plates. Not once in their several years of working together did either Watson or Crick touch or look directly at a fiber of DNA. They did not have to: Avery, Chargaff, Asbury, Wilkins, and Franklin already had done this part of the process for them” (p. 224).

However, the general first reaction toward theories is suspicion, doubt, and disbelief. Richard Feynman is considered by many as one of the greatest theoretical physicists of the second half of the 20^{th} century. Mark Kac wrote on Feynman:

“There are two kinds of geniuses: the ‘ordinary’ and the ‘magicians.’ An ordinary genius is a fellow whom you and I would be just as good as, if we were only many times better. There is no mystery as to how his mind works. Once we understand what they've done, we feel certain that we, too, could have done it. It is different with the magicians. Even after we understand what they have done, it is completely dark. Richard Feynman is a magician of the highest calibre.”

The same Feynman writes in his book “Surely You're Joking Mr. Feynman!”:

“I've very often made mistakes in my physics by thinking the theory isn't as good as it really is, thinking that there are lots of complications that are going to spoil it—an attitude that anything can happen, in spite of what you're pretty sure should happen” (underline added).

Even the great Feynman was suspicious of theories.

Another example is the reaction of the scientific community to atomic theory. According to Albert Einstein (underline added):

“The antipathy of these scholars towards atomic theory can indubitably be traced back to their positivistic philosophical attitude. This is an interesting example of the fact that even scholars of audacious spirit and fine instinct can be obstructed in the interpretation of facts by philosophical prejudices. The prejudice—which has by no means dies out in the meantime—consists in the faith that facts by themselves can and should yield scientific knowledge without free conceptual construction” (Einstein 1951²¹⁶, p. 49).

The oppenents of the atomic theory suggested avoiding the lines, dots are enough.

However, according to Henri Poincare, one of the greatest mathematicians of the early 20^{th} century:

“Science is built of facts as a house is built of bricks; but an accumulation of facts is no more science than a pile of bricks is a house” (from La Science et L'hypothese).

2. Technical Note: Microcompetition

a) Introduction

(1) The Problem

If, after disturbances, a system always returns to the same equilibrium, the equilibrium is called “stable.” Let “good health” be identified with a certain stable equilibrium. Any stable equilibrium different from “good health” will be called “chronic disease.” Exogenous events that produce new stable equilibria will be called “disruptions.” Specifically, the exogenous events that move a biological system from “good health” to “chronic disease” are disruptions. The disruptions responsible for most of the chronic diseases, such as, cancer, obesity, osteoarthritis, atherosclerosis, multiple sclerosis, type II and type I diabetes, and male pattern baldness, are mostly unknown. Moreover, even in cases where a disruption is identified, the molecular effects associated with the disruption, and the sequence of events leading from the disrupted molecular environment to clinical symptoms is unknown. The examples section identifies a single disruption responsible for many of the chronic diseases inflicting human kind, and present the sequence of events leading from the disruption to observed molecular and clinical effects.

(2) Framework and Symbolic Language

The examples section adopts the following framework. The first section of every subject presents conceptual building blocks. The section introduces variables used in following sections. Every variable is associated with a measure, that is, all variables are quantitative in nature. The second section presents a model that uses the introduced variables. Every model describes a sequence of quantitative events. The following symbolic presentation illustrates a sequence of quantitative events. [symbol]
A→↑[B]→↓[C]→↑D
Sequence of quantitative events 1: Symbolic example.

The letters A to D represent events. Events in brackets show a range of values. Events without brackets show only two values “occur” and “not occur” where “occur” is considered higher than “not occur” (see note below). An arrow facing up or down illustrates an increase or decrease in value, respectively. A boxed arrow facing up or down indicates an exogenous event. An arrow facing right means “leads to.” The above sequence of quantitative events should be read as follows: an exogenous event increases the value of A, which leads to an increase in B, which, in turn, leads to a decrease in C, which leads to an increase in D. A sequence of quantitative events is equivalent to the traditional concept of biological pathway with an added emphasis on the quantitative changes resulting from an exogenous event.

Notes:

1. Brackets can indicate rate, concentration, intensity, probability, etc. Therefore, an arrows facing up next to an event in brackets can indicate increase in concentration, in intensity, etc.

2. An arrow facing up next to an event without brackets indicates a switch from a “not occur” to “occur,” for instance, before and after administration of a treatment, before and after transfection, etc.

3. Exogenous events are sometimes called interventions. Examples of exogenous events are mutations, treatments, infections, etc.

In principle, every two events in a sequence of quantitative events can be represented as relation between a dependent variable and an independent variable. Consider the following function.

$$\begin{matrix}D = & f ( A ) \\ & ( + )\end{matrix} \qquad Function 2$$

The symbol D denotes the dependent variable and A the independent variable. The (+) sign under A denotes a positive, or direct relation, that is, an increase in A increases D. A (−) sign denotes a negative or inverse relation.

Note: The dependent variable is always “down stream” from the independent variable.

A set of chains of quantitative events (i.e. multiple pathways), which converge at the same variable, can be represented as a relation between a dependent variable and set of independent variables. Consider the following function.

$$\begin{matrix}y = & f ( {x}_{1} , & … , & {x}_{n} ) \\ & ( + ) & & ( - )\end{matrix} \qquad Function 3$$

The letter “y” denotes the dependent variable, and the letters x₁ to xₙ represent n independent variables. As above, the (+) sign under x₁ denotes a positive relation, and the (−) sign under xₙ denotes a negative relation.

The third section in the adopted framework presents the derived predictions and compares the predictions to empirical observations reported in the scientific literature. The fourth section presents conclusions.

b) Microcompetition for a Limiting Transcription Complex

(1) Conceptual Building Blocks

Let DNA₁ and DNA₂ be two DNA sequences, which bind the transcription complexes Complex₁ and Complex₂, respectively. DNA₁ and DNA₂ will be called microcompetitors if Complex₁ and Complex₂ include the same transcription factor. A special case of microcompetition is two sequences that bind the same transcription complex.

Assume the transcription factor f transactivates gene G. Let f_{active} denote the “f” forms, which can bind G (that is, any other form cannot bind G). “f” will be called limiting with respect to G, if any decrease in the concentration of f_{active}, decreases G transcription. Note that the definition does not suggest that every increase in the concentration of f_{active} increases G transcription. An increase in concentration can increase binding of “f” to G. However, such binding might be insufficient for transactivation.

Note: The technical note on definitions presents more definitions of microcompetition and other fundamental concepts.

(2) Model

Let G denote a gene that is stimulated or suppressed by a transcription complex C, [mRNA_{G}], the concentration of G mRNA (brackets indicate concentration, or probability of detecting the molecule using a certain measurement procedure), [DNA_{G}], the copy number of the G DNA sequence that binds C, [DNAₒₜₕₑᵣ], the copy number of other DNA sequences that also bind C, and Affinity_{other/G,} the affinity of other DNA to C relative to the affinity of G DNA sequences to C.

Assume the cellular availability of at least one of the factors comprising the transcription complex C is limiting. Then, the effect of microcompetition on the level of transcription of the gene G can be presented using the following function (referred to as the microcompetition function, denoted f_{MC}, or microcompetition model). Note that the function can be applied to a gene either stimulated or suppressed by the transcription complex.

$$\begin{matrix}[ {mRNA}_{G} ] = & {f}_{MC} ( [ {DNA}_{G} ] , & [ {DNA}_{other} ] , & {Affinity}_{other / G} ) \\ C stimulated / & ( + ) / ( - ) & ( - ) / ( + ) & ( - ) / ( + ) \\ suppressed gene & & & \end{matrix} \qquad Funtion 4$$

Assume other variables are fixed. Then, an increase in copy number of “other DNA” decreases expression of the cellular gene G. Moreover, if “other DNA” has high affinity to the limiting complex, the decrease in expression might be substantial even for a small copy number of “other DNA.”

(3) Prediction

Let plasmid_{A} and plasmid_{B} present two plasmids that express gene_{A} and gene_{B} following binding of transcription complex C_{A} and C_{B}, respectively. Also, assume limiting availability of at least one of the factors comprising C_{A} and C_{B}, and fixed copy number of plasmid_{A}. Then, an increase in copy number of plasmid_{B} decreases expression of gene_{A}.

(4) Observations

(a) Scholer 1984

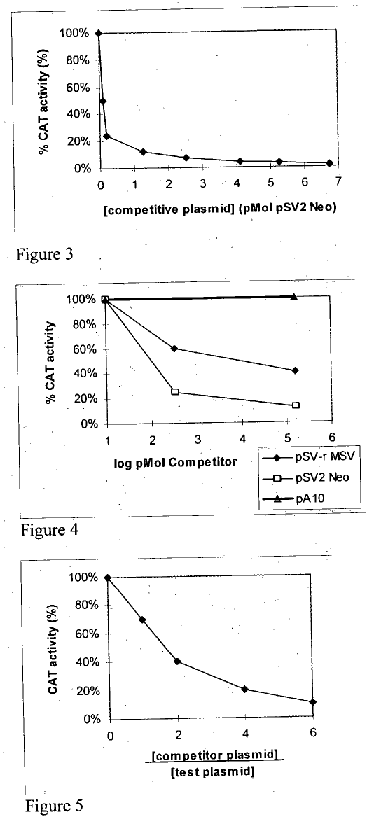

The plasmid pSV2CAT expresses the chloramphenicol acethyltransferase (CAT) gene under control of the SV40 promoter/enhancer. A study (Scholer 1984²¹⁷) first transfected increasing amounts of pSV2CAT in CV-1 cells. CAT activity reached a plateau at 0.3-pmol pSV2CAT DNA per dish. Based on this observation, the study concluded that CV-1 cell contain a limited concentration of cellular factor needed for pSV2CAT transcription. Next, the study cotransfected a constant concentration of pSV2CAT with increasing concentrations of pSV2neo, a plasmid identical to pSV2CAT, except the reporter gene is neomycin-phosphotransferase (neo). FIG. 3 presents the observations (Scholer 1984, ibid, FIG. 2B).

The addition of pSV2neo decreased CAT activity. Next, the study cotransfected pSV2CAT with pA10, a plasmid that includes all SV40 early control elements except for the 72-bp enhancer. No competition was observed. A point mutation in the 72-bp enhancer, which abolished the enhancer functional activity, also eliminated competition. Based on these observations, Scholer, et al., (1984, ibid) concluded: “taken together, our data indicate that a limited amount of the cellular factors required for the function of the SV40 72-bp repeats is present in CV-1 cells. Increasing the number of functional SV40 enhancer elements successfully competes for these factors, whereas other elements necessary for stable transcription did not show such an effect.” The study also observed competition between pSV2CAT and pSV-rMSV, a plasmid, which harbors the Moloney murine sarcoma virus (MSV) enhancer. Consider the FIG. 4 (Scholer 1984, ibid, FIG. 5A, see also 5B).

Note, that except for the enhancers, the transcriptional control elements in pSV2CAT and pSV-rMSV are the same. Based on these observations, Scholer, et al., (1984, ibid) concluded: “one class of (a limiting) cellular factor(s) is required for the activity of different enhancers. Furthermore, BK (BK virus) and RSV (Rous sarcoma virus) enhancers also interact with the same class of molecule(s).”

(b) Mercola 1985

The plasmid pSV2CAT expresses the chloramphenicol acethyltransferase (CAT) gene under control of the SV40 promoter/enhancer. The pX1.0 plasmid contains the murine immunoglubulin heavy-chain (Ig H) enhancer. The pSV2neo expresses the neo gene under control of the SV40 promoter/enhancer. The pA10neo and pSV2neo are identical except that pA10neo lacks most of the SV40 enhancer.

A study (Mercola 1985²¹⁸) cotransfected a constant amount of pSV2CAT into murine plasmacytoma P3X63-Ag8 cells as test plasmid, with increasing amounts of pX1.0 as competitor plasmid. A plasmid lacking both reporter gene and enhancer sequences was added to produce equimolar amounts of plasmid DNA in the transfected cells. FIG. 5 illustrates the observed relative CAT activity as a function of the relative concentration of the competitor plasmid (Mercola 1985, ibid, FIG. 4A).

An increase in concentration of the cotransfected murine immunoglubulin heavy-chain (H) enhancer decreased expression from the plasmid carrying the SV40 viral enhancer. Microcompetition between viral and cellular heavy-chain enhancers decreased expression of the gene under control of the viral enhancer. Based on these observations, Mercola, et al., (1985, ibid) concluded that in the plasmacytoma cells the heavy chain enhancer competes for a trans-acting factor required for the SV40 enhancer function.

In another experiment, the study cotransfected a constant amount of pSV2CAT, as test plasmid, with increasing amount of pSV2neo, as competitor plasmid, in Ltk- or ML fibroblast cells. To isolate the effect of the viral enhancer, the study also cotransfected a constant amount of the test plasmid pSV2CAT with increasing amount of the enhancerless pA10neo plasmid. FIG. 6 illustrates the observed relative CAT activity as a function of the relative concentration of the competitor plasmid (Mercola 1985, ibid, FIG. 4B).

An increase in concentration of the cotransfected SV40 viral enhancer decreased expression from the plasmid also carrying the SV40 enhancer. An increase in concentration of a plasmid lacking the enhancer did not affect the reporter gene activity of the test plasmid.

Overall, the study concluded: “in vivo competition experiments revealed the presence of a limited concentration of molecules that bind to the heavy-chain enhancer and are required for its activity. In the plasmacytoma cell, transcription dependent on the SV40 enhancer was also prevented with the heavy-chain enhancer as competitor, indicating that at least one common factor is utilized by the heavy-chain and SV40 enhancers.”

(c) Scholer 1986

A study (Scholer 1986²¹⁹) cotransfected CV-1 monkey kidney cells with a constant amount of a plasmid containing the human metallothionein II (hMT-II_{A}) promoter (−286, +75) fused to the bacterial gene encoding chloramphenicol acetyltransferase (hMT-HA-CAT) along with increasing concentrations of a plasmid containing the viral SV40 early promoter and enhancer fused to the bacterial gene conferring neomycin resistance (pSV2neo). FIG. 7 presents the observed relative CAT activity (expressed as the ratio between CAT activity in the presence of pSV2neo and CAT activity in the absence of pSV2neo) as a function of the molar ratio of pSV2Neo to hMT-HA-CAT (Scholer 1986, ibid, FIG. 2).

The figure illustrates the effect of competition between the two plasmids on relative CAT activity. A 2.4-fold molar excess of the plasmid containing the viral enhancer decreased CAT activity by 90%. No competition was observed with the viral plasmid after deletion of the SV40 enhancer suggesting that elements in the viral enhancer are responsible for the observed decrease in reporter gene expression.

(d) Adam 1996

A study (Adam 1996²²⁰) transiently cotransfected JEG-3 human choriocarcinoma cells with a constant amount of plasmid carrying the platelet derived growth factor-B (PDGF-B) promoter/enhancer-driven CAT reporter gene (pPDGF-B-CAT), and increasing amounts of a plasmid containing either the human cytomegalovirus promoter/enhancer fused to β-galactosidase (pCMV-βgal), or the SV40 early promoter and enhancer elements fused to βgal (pSV40-βgal). Assume that the PDGF-B, CMV, and SV40 promoters/enhancers bind the same limiting transcription complex, and that the complex stimulates PDGF-B transcription. According to microcompetition model, an increase in pCMV-βgal or pSV40-βgal should decrease CAT expression. FIG. 8 presents the observations.

The observations demonstrate the negative effect of microcompetition between the viral enhancer and PDGF-B on relative CAT activity. As predicted, the effect is concentration-dependent.

(e) Hofman 2000

The pSG5 plasmid includes the early SV40 promoter to facilitate in vivo expression, and the T7 bacteriophage promoter to facilitate in vitro transcription of cloned inserts. Both the pcDNA1.1 and pIRESneo plasmids include the human cytomegalovirus (CMV) immediate early (1E) promoter and enhancer.

A study (Hofman 2000²²¹) constructed a series of pSG5-based vectors by cloning certain sequences into the EcoRI restriction site (“insert plasmid,” see list in table below). The inserts varied in length measured in base pair (bp). The study cotransfected each insert plasmid (650 ng) with pSG5-luc (20 ng) as test plasmid in COS-7 cells. The test plasmid pSG5-luc was also cotransfected with the pGEM-7Zf(+) plasmid, or with herring sperm DNA. Luciferase (luc) activities were measured. Luc activity in presence of the empty pSG5 vector was arbitrarily set to 1. Table 1: presents the observed relative luc activity in every experiment (Hofman 2000, ibid, FIG. 3a).

Based on these observations, Hofman, et al, (2000, ibid) concluded: “Remarkably, the measured luciferase activity tended to be inversely related to the length of the insert in the cotransfected pSG5-constructs.” Moreover, “We can conclude from these data that the SV40 promoter driven expression of nuclear receptor or of luciferase in COS-7 cells is inhibited to various degrees by cotransfection, with maximal inhibition in the presence of the empty expression vector and minimal inhibition in the presence of pSG5 constructs containing large inserts.” First, note that the pGEM-7Zf(+) plasmid and the herring sperm DNA do not include a human viral promoter or enhancer. The promoter in pGEM-7Zf(+) includes the bacteriophage SP6 and bacteriophage T7 RNA polymerase promoters (a bacteriophage is a virus that infects bacteria). Second, note that a decrease in the size of the insert increases the copy number of the insert plasmid resulting in accentuated microcompetition with the test plasmid.

TABLE 1
Observed effect of pSG5-based vectors with different
size inserts on pSG5-luc expression.

| | | Luc activity |
|---|---|---|
| | Size of | from pSG5-luc |
| Plasmid | insert (bp) | (fold increase) |
| pGEM7zf+ | | 72 |
| herring | | 71 |
| pSG5-NuRIP183 | 4,776 | 47 |
| pSG5-TIF2 | 4,395 | 40 |
| pSG5-NuRIP183D1 | 4,326 | 36 |
| pSG5-NuRIP183D2 | 3,723 | 33 |
| pSG5-NuRIP183D3 | 3,219 | 30 |
| pSG5-NuRIP183D4 | 2,684 | 28 |
| pSG5-NuRIP183D5 | 2,400 | 25 |
| pSG5-NuRIP183D6 | 1,889 | 22 |
| pSG5-ARA70 | 1,800 | 20 |
| pSG5-TIF2.5 | 738 | 7 |
| pSG5-DBI | 259 | 3 |
| pSG5 | 0 | 1 |

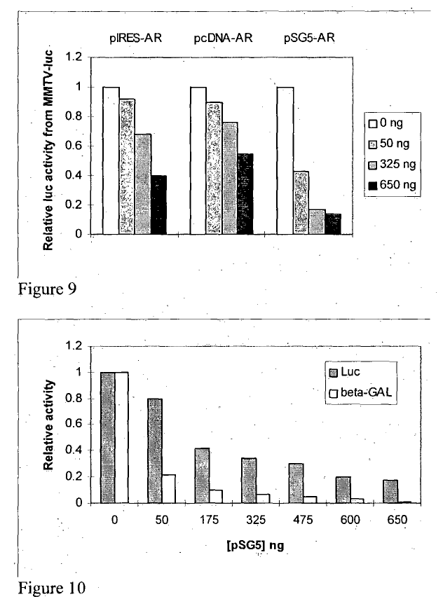

The study also measured the effect of cotransfection on the activity of the androgen receptor (AR). The study transfected COS-7 cells with 20 ng pIRES-AR, pcDNA-AR or pSG5-AR plasmids which express AR, 500 ng MMTV-luc which highly expresses luc following AR stimulation of the MMTV promoter, and increasing amounts of the empty pSG5 vector. The pGEM-7Zf(+) plasmid was used instead of the expression plasmid to maintain a 650 ng final concentration of cotransfected DNA. Transfected cells were treated with 10 nM R1881, an AR ligand, and luciferase activity was measured. The luc activity in the presence of 650 ng pGEM-7Zf(+) was arbitrarily set to 1, and the relative luc activity was calculated. FIG. 9 presents the results (Hofman 2000, ibid, FIG. 5a).

According to Hofman, et al., (2000, ibid): “The MMTV-luciferase response was strongly decreased in the presence of increasing concentrations of the empty expression vector and the decreased receptor activities were proportional to AR expression levels.” The decrease in MMTV-luc transcription resulted from decreased transcription of the AR gene expressed by the pIRES-AR, pcDNA-AR, and pSG5-AR plasmids (see also Hofman 2000, ibid, FIG. 5b). Transfection with the calcium phosphate precipitation method, instead of FuGENE-6™, produced similar results.

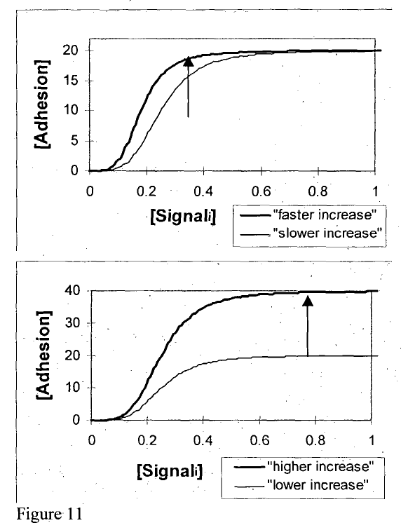

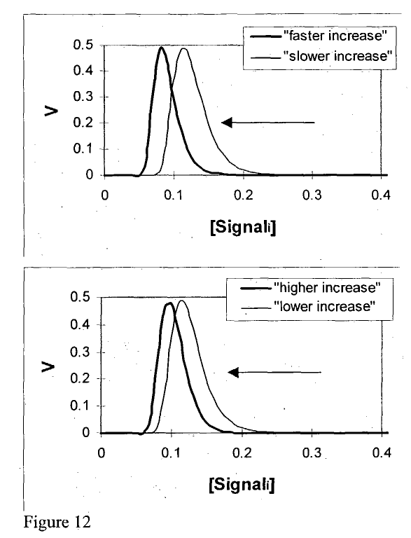

Finally, the study transiently cotransfected COS-7 cells with 20 ng pSG5-AR, 20 ng pS40-β-galactosidase (βGAL), 20 ng pSG5-luc, and increasing amounts of the empty pSG5 vector pGEM-7Zf(+) was used to maintain the DNA concentration at a constant level. Luc and βGAL activities in the presence of 650 pGEM-7Zf(+) were arbitrarily set to 1, and relative βGAL and luc activities were calculated following treatment with 10 nM R1881. FIG. 10 presents the results (Hofman 2000, ibid, FIG. 7a).

Based on these observations, Hofman, et al, (2000, ibid) concluded: “The most likely explanation is that the total amount of transfected expression vectors largely exceeds the capacity of the transcriptional machinery of the cell. For that reason, competition occurs between the receptor construct and the cotransfected construct.”

c) p300 and GABP

(1) Conceptual Building Blocks

(a) GABP Transcription Factor

The DNA motif (A/C)GGA(A/T)(G/A), termed the N-box (or the ETS binding site, denoted EBS), is the core binding sequence of the transcription factor known as GA Binding Protein or GABP, Nuclear Respiratory Factor 2 (NRF-2)¹, E4 Transcription factor 1 (E4TF1)(Watanabe 1988)²²²,² and Enhancer Factor 1A (EF-1A). For simplicity, let us refer to the transcription factor as GABP, and the DNA binding motif as N-box.
¹ Nuclear Respiratory Factor 2 should not be confused with NF-E2 Related Factor 2 which is also abbreviated NRF2 or NRF-2. ² The transcription factor binds to the promoter of the adenovirus early-region 4 (E4). Hence the name E4 transcription factor I. ³ Enhancer Factor 1A should not be confused with Elongation Factor 1A which is also abbreviated EF-1A.

Five subunits of GABP are known, GABPα, GABPβ 1 and GABPβ2 (together called GABPβ), GABPγ1 and GABPγ2 (together called GABPγ). GABPα is an ets-related DNA-binding protein, which binds the N-box. GABPα forms a heterocomplex with GABPβ, which stimulates transcription efficiently both in vitro and in vivo. GABPα also forms a heterocomplex with GABPγ. The degree of transactivation by GABP appears to be a result of the relative intracellular concentrations of GABPβ and GABPγ. An increase in GABPβ relative to GABPγincreases transcription, while an increase of GABPγ relative to GABPβ decreases transcription. The degree of transactivation by GABP is, therefore, a function of the ratio between GABPβ and GABPγ. Control of the ratio within the cell regulates transcription of genes with binding sites 19223) for GABP (Suzuki F 1998).

(b) Cellular DNA Binds GABP

GABP binds promoters and enhancers of many cellular genes including β₂ leukocyte integrin (CD18) (Rosmarin 1998²²⁴), interleukin 16 (IL-16) (Bannert 1999²²⁵), interleukin 2 (IL-2) (Avots 1997²²⁶), interleukin 2 receptor 13-chain (IL-2Rβ) (Lin, 1993²²⁷), IL-2 receptor γ-chain (IL-2 γc) (Markiewicz 1996²²⁸), human secretory interleukin-1 receptor antagonist (secretory IL-1ra) (Smith 1998²²⁹), retinoblastoma (Rb) (Sowa 1997²³⁰), human thrombopoietin (TPO) (Kamura 1997²³¹), aldose reductase (Wang 1993²³²), neutrophil elastase (NE) (Nuchprayoon 1999²³³, Nuchprayoon 1997²³⁴), folate binding protein (FBP) (Sadasivan 1994²³⁵), cytochrome c oxidase subunit Vb (COXVb) (Basu 1993²³⁶, Sucharov 1995²³⁷), cytochrome c oxidase subunit IV (Carter 1994²³⁸, Carter 1992²³⁹), mitochondrial transcription factor A (mtTFA) (Virbasius 1994²⁴⁰), β subunit of the FoF1 ATP synthase (ATPsynβ) (Villena 1998²⁴¹), prolactin (prl) (Ouyang 1996²⁴²) and the oxytocin receptor (OTR) (Hoare 1999²⁴³) among others.

(c) Viral DNA Binds GABP

The N-box is the core binding sequence of many viral enhancers including the polyomavirus enhancer area 3 (PEA3) (Asano 1990²⁴⁴), adenovirus E 1A enhancer (Higashino 1993²⁴⁵), Rous Sarcoma Virus (RSV) enhancer (Laimins 1984²⁴⁶), Herpes Simplex Virus 1 (HSV-1) (in the promoter of the immediate early gene ICP4) (LaMarco 1989²⁴⁷, Douville 1995²⁴⁸), Cytomegalovirus (CMV) (IE-1 enhancer/promoter region) (Boshart 1985²⁴⁹), Moloney Murine Leukemia Virus (Mo-MuLV) enhancer (Gunther 1994²⁵⁰), Human Immunodeficiency Virus (HIV) (the two NF-κB binding motifs in the HIV LTR) (Flory 1996²⁵¹), Epstein-Barr virus (EBV) (20 copies of the N-box in the +7421/+8042 oriP/enhancer) (Rawlins 1985²⁵²) and Human T-cell lymphotropic virus (HTLV) (8 N-boxes in the enhancer (Mauclere 1995²⁵³) and one N-box in the LTR (Kornfeld 1987²⁵⁴)). Moreover, some viral enhancers, for example SV40, lack a precise N-box, but still bind the GABP transcription factor (Bannert 1999, ibid).

Many studies showed binding of GABP to the N-boxes in these viral enhancers. For instance, Flory 1996 (ibid) showed binding of GABP to the HIV LTR, Douville 1995 (ibid) showed binding of GABP to the promoter of ICP4 of HSV-1, Bruder 1991²⁵⁵ and Bruder 1989²⁵⁶ showed binding of GABP to the adenovirus E1A enhancer element I, Ostapchuk 1986²⁵⁷ showed binding of GABP (called EF-1A in the paper) to the polyomavirus enhancer and Gunther 1994 (ibid) showed binding of GABP to Mo-MuLV.

Other studies demonstrated competition between these viral enhancers and enhancers of other viruses. For instance, Scholer 1984 (ibid) showed competition between the Moloney Sarcoma Virus (MSV) enhancer and SV40 enhancer, and competition between the RSV enhancer and the BK virus enhancer.

(d) p300·GABP is Limiting

The coactivator p300 is a 2,414-amino acid protein initially identified as a binding target of the E1A oncoprotein. cbp is a 2,441-amino acid protein initially identified as a transcriptional activator bound to phosphorylated cAMP response element (CREB) binding protein (hence, cbp). p300 and cbp share 91% sequence identity and are functionally equivalent. Both p300 and cbp are members of a family of proteins collectively referred to as p300.

Note: Some papers prefer the notation “p300/cbp,” however; the specifications sometimes use “p300” to represent the entire family of proteins.

Although p300 and cbp are widely expressed, their cellular availability is limited. Several studies demonstrated inhibited activation of certain transcription factors resulting from competitive binding of p300 to other cellular or viral proteins. For example, competitive binding of p300, or cbp, to the glucocorticoid receptor (GR), or the retinoic acid receptor (RAR), inhibited activation of a promoter dependent on the AP-1 transcription factor (Kamei 1996²⁵⁸). Competitive binding of cbp to STAT1α inhibited activation of a promoter dependent on both the AP-1 and ETS transcription factors (Horvai 1997²⁵⁹). Competitive binding of p300 to STAT2 inhibited activation of a promoter dependent on the NF-κB RelA transcription factor (Hottiger 1998²⁶⁰). Other studies also demonstrated limited availability of p300, see, for instance, Pise-Masison 2001²⁶¹, Banas 2001²⁶² Wang C 2001²⁶³, Ernst 2001²⁶⁴, Yuan 2001²⁶⁵, Ghosh 2001²⁶⁶ Li M 2000²⁶⁷, Nagarajan 2000²⁶⁸, Speir 2000²⁶⁹, Chen Y H 2000²⁷⁰, and Werner 2000²⁷¹.

GABPα directly binds the C-terminus of the p300 acetyltransferase (Bush 2003²⁷², Bannert 1999, ibid). Since p300 is limiting, the transcription complex p300·GABP is also limiting.

(2) Conclusion

A virus that binds GABP will be called GABP virus. Let g-GABP denote a cellular GABP regulated gene, and v-GABP, a GABP virus. Since DNA_{G-GABP} and DNA_{v-GABP} are special cases of DNA_{G} and DNAₒₜₕₑᵣ, respectively, the effect of microcompetition on g-GABP transcription can be represented using the f_{MC} function above.

$$\begin{matrix}[ {mRNA}_{G \text{-} GABP} ] = {f}_{MC} ( [ {DNA}_{G \text{-} GABP} ] , [ {DNA}_{v \text{-} GABP} ] , {Affinity}_{v / G} ) \\ \begin{matrix}GABP \\ stimulated \text{/} suppressed \\ gene\end{matrix} ( + ) \text{/} ( - ) ( - ) \text{/} ( + ) ( - ) \text{/} ( + )\end{matrix} \qquad Function 5$$

Microcompetition for p300·GABP between DNA of a GABP virus and DNA of a cellular GABP regulated gene decreases availability of p300·GABP to the cellular gene. If p300·GABP stimulates transcription of the cellular gene, the virus decreases transcription. If p300·GABP suppresses transcription, the virus increases transcription. The same conclusion holds for other types of foreign DNA sequences that bind GABP.

3. Technical Note: Transefficiency

4). Principle

(1) Definition: Transefficiency (TransE)

Consider a gene G. Assume the transcription factor F, binds Box_{G} in the promoter/enhancer of G. Let the function “f” represent the relation between [mRNA_{G}] and [F₁·Box_{G}].
[mRNA_{G}]=f([F₁·Box_{G}]) Function 6

Define transefficiency of F₁, denoted TransE(F₁), as follows:

$$TransE ( {F}_{1} ) = \frac{ⅆ [ {mRNA}_{g} ]}{ⅆ [ {F}_{1} \cdot {Box}_{g} ]} \qquad Function 7$$

Transefficiency of F₁ in G transcription is defined as the local effect of [F₁·Box_{G}] on [mRNA_{G}], and is equal to the slope of the curve representing “f” at a certain point (derivative).

Notes:

1. If “f” is non-linear, for instance, S-shaped, transefficiency can be different at different F₁ concentrations.

2. If F₁ is a transactivator of G, transefficiency of F₁ is positive. If F₁ is a suppressor, transefficiency is negative.

(2) Conclusion: Transefficiency-Mediated Suppression

Consider a gene G and Cellᵢ. Let F₁ and F₂ denote two transcription factors. Assume the following conditions.
(1) In isolation, F₁ and F₂ transactivate G transcription, that is,
TransE(F₁)>0 and TransE(F₂)>0
(2) F₁ and F₂ compete for binding to the G promoter/enhancer
(3) Cellᵢ expresses both F₁ and F₂
(4) In a<[F₁·Box_{G}]<b and c<[F₂·Box_{G}]<d, TransE(F₁)<TransE(F₂)

Then, an increase in binding of F₁ to Box_{G}, in the range (a, b), decreases G transcription in Cellᵢ. An increase in binding of F₁ to Box_{G} decreases binding of F₂ to the DNA box. Since F₁ is less transefficient then F₂, the net effect of the increase in [F₁·Box_{G}] is a decrease in G transcription. In isolation, F₁ is a transactivator of G. However, in Cellᵢ, which expresses both F₁ and F₂, F₁ is a suppressor of G transcription.

Notes:

1. An increase in binding of the more transefficient factor increases transcription both when isolated, or in presence of the other factor. The different environments only modify the rate of change in transcription, not the direction. In contrast, the less transefficient factor will show transactivation only when isolated from the other factor.

2. TransE(F₁)=0 and TransE(F2)>0 is a special case of condition (4).

3. If TransE(F₁)<0, F₁ is a suppressor of G transcription in isolation and in presence of F₂. However, in presence of F₂, F₁ shows stronger suppression compared to an environment where F₁ is isolated from F₂. In other words, presence of F₂ results in a steeper negative slope of the curve that represents the relation between [mRNA_{G}] and [F₁·Box_{G}].

b) Examples

(1) CD18 (β₂ Integrin)

(a) Condition (1): Two Transactivators in Isolation

(i) PU.1

Rosmarin 1995A²⁷³ identified two PU.1 consensus binding-sites in the CD18 promoter, a distal site at (−75, −70), and a proximal site at (−55, −50). Constructs containing mutations at either site showed decreased CD18 promoter activity in U-937 transfected cells. U-937 nuclear extracts and in vitro translated PU.1 showed binding to the (−85, −37) region of the CD18 promoter.

Li S L 1999²⁷⁴ generated the pGL3-CD18-81 plasmid, which expresses the luciferase reporter construct under control of the first 81 nucleotides of the CD18 promoter, and pGL3-CD18-81-76T77A, a variation plasmid, which includes T and A instead of residues 76G and 77T in the wild-type CD18 promoter, respectively. The study transiently expressed the plasmids in THP-1 cells and measured the reporter gene expression. The results showed a 75% decreased activity of the mutated relative to the wild-type promoter. The study also compared PU.1 binding to a probe containing the first wild-type 81 nucleotides, and a probe, which included the T and A mutation. The resulted showed PU.1 binding to the wild-type promoter, and little or no binding to the mutated probe.

Panopoulos 2002²⁷⁵ cultured 32D.ER-S3 myeloid cells, expressing the EpoR engineered to activate Stat3 instead of Stat5, in IL-3 or Epo-containing medium. Cells in IL-3-containing medium showed low levels of CD18 expression, and increased CD18 expression in Epo-containing medium. The cells also showed low PU.1 expression in IL-3-containing medium, and increased PU.1 mRNA in Epo-containing medium. To examine the relation between cytokines and PU.1, the study generated a dominant inhibitory isoform of PU.1 (PU.1-TAD) by deleting residues 33-100 from the PU.1 transactivation domain. The study, then, transfected PU.1-TAD in 32D.ER-S3 cells, cultured the cells in Epo-containing medium, and measured CD18 expression in PU.1-TAD transfected and non-transfected cells. The results showed decreased CD18 expression in PU.1-TAD transfected cells compared to non-transfected cells.

The observations in Rosmarin 1995A (ibid), L1 SL 1999 (ibid), and Panopoulos 2002 (ibid), indicate that PU.1 is a transactivator of CD18

(ii) GABP

A study (Rosmarin 1995B²⁷⁶) showed binding of GABP to the (−85, −37) region of the CD18 promoter, specifically, to the three ETS binding sites at (−75, −72), (−53, −50), and (−47, −44). Mutation of the ETS binding sites inhibited GABP binding. To examine the effect of GABP on CD18 transcription, the study used HeLa cells, which show no expression of PU.1. The cells were transfected with 20 μg of a CD18 plasmid (−918/luc), 5 μg of a GABPα plasmid (pCAGGS-E4TF1-60), and 5 μg of a GABPβ plasmid (pCAGGS-E4TF1-53). The internal control was CMV/hGH (1 μg). The study added pGEM3zf- to bring the amount of transfected DNA to 40 μg. The results showed a “modest effect” of GABP on CD18 promoter activity, about 2.5-fold increase in activity in cells transfected with GABP+CD18+CMV/hGH compared to cells transfected with CD18+CMV/hGH only.

Note: The pCAGGS vector contains the CMV enhancer (Niwa 1991²⁷⁷). Therefore, the increase in CMV concentration in the GABP transfected cells (5+5+1 μg in GABP transfected cells vs. 1 μg in cell transfected with the internal control only) increases microcompetition with the internal control (CMV/hGH), which decreases expression of the GH reporter gene, and increases the expression of luc measured in relative terms. Luc expression shows an increase in relative terms even if there is no increase in actual luc concentration. In light of the microcompetition effect on the internal control, the question is what drives the increase in relative luc expression, the GABP transactivators, microcompetition between the CMV promoters, or both. (Similar issues apply to the other results reported in Rosmarin 1995B, ibid, FIG. 7).

Another study (Rosmarin 1998, ibid) transfected Drosophila Schneider cells with 5 μg of a CD18 plasmid (−96/luc), 2.5 μg of a GABPα plasmid (pPac-GABPα), and 2.5 μg of a GABPα plasmid (pPac-GABPβ), or 5 μg of the CD18 plasmid alone as control. The results showed 11-fold increase in CD18 promoter activity in cells transfected with GABP compared to controls.

Notes:

1. Schneider cells lack endogenous PU.1 activity (Muller S 1999²⁷⁸), and therefore, constitute an “in isolation” environment for GABP.

2. The study uses no internal control, and therefore, avoids the issues mentioned above.

Another study (Bottinger 1994²⁷⁹) showed binding of two transcription factors, one related to GABP, the other to PU.1, to two DNA boxes, (−0.81, −68) and (−55, −41), in the CD18 promoter.

The observations in Rosmarin 1995B (ibid), Rosmarin 1998 (ibid), and Bottinger 1994 (ibid), indicate that GABP is a transactivator of CD18.

(b) Condition (2): Competition for Same DNA Site

Rosmarin 1995B (ibid) showed that GABP and PU.1 compete for binding to the same DNA sites in the CD18 promoter (Rosmarin 1995B, ibid, FIGS. 6A and B).

(c) Condition (3): Cells with Dual Expression

PU.1 is expressed in macrophages, mast cells, B cells, neutrophils, and hemopoietic stem cells. The same cells also express GABP.

(d) Condition (4): Different Transefficiency

There are no direct observations (to the best of my knowledge), which show different transefficiency of PU.1 and GABP in CD18 transcription in monocytes/macrophages. However, some arguments support the conclusion that PU.1 is more transefficient than GABP.

1. Differentiation

Several studies showed that PU.1 is necessary for the development of myeloid progenitor-derived monocytes (Anderson 1999²⁸⁰, DeKoter 1998²⁸¹ Anderson 1998²⁸²), and dendritic cells (Anderson 2000²⁸³, Guerriero 2000²⁸⁴). Moreover, expression of PU.1 increases during differentiation of monocytes (Cheng 1996²⁸⁵, FIG. 4C, Voso 1994²⁸⁶, FIG. 1). In the intima, monocytes differentiate into macrophages and increase the expression of CD18 (see chapter on atherosclerosis, p 161). Therefore, in the intima, an increase in PU.1 expression in monocytes correlates with an increase in CD18 expression.

2. Redox

An increase in oxidative stress decreases binding of GABP to DNA (Chinenov 1998²⁸⁷). Since the regions susceptible to redox regulation in GABP are not highly conserved in PU.1, PU.1 binding to DNA is, most likely, redox independent. Moreover, PU.1 is an essential transactivator of the cytochrome b heavy chain (gp91-phox), which is the redox center of the NADPH-oxidase system (Islam 2002²⁸⁸, Voo 1999²⁸⁹, Suzuki S 1998²⁹⁰). Macrophages and macrophage-turned foam cells in atherosclerotic plaque show high expression of gp91-phox (Kalinina 2002²⁹¹). Therefore, the gp91-phox promoter, most likely, maintains PU.1 binding under oxidative rich conditions, consistent with the above conclusion. Since only GABP is redox sensitive, the increase in oxidative stress in macrophages-turned foam cells decreases GABP binding to the CD18 promoter, which increases PU.1 binding. Therefore, in intimal macrophages, an increase in PU.1 binding to DNA is correlated with an increase in CD18 expression.

Both arguments indicate that PU.1 is more transefficient than GABP in transactivating the CD18 promoter in monocytes/macrophages.

(e) Conclusion

According to transefficiency-mediated suppression, an increase in GABP binding to the CD18 promoter/enhancer decreases CD18 transcription. The same holds for the opposite direction, a decrease in GABP binding to the CD18 promoter/enhancer increases CD18 transcription.

(2) CD49d (α₄ Integrin)

A study (Rosen 1994²⁹²) showed that GABP and another ets-related factor bind the same region in the CD49d promoter/enhancer. Although details are missing, based on the observations reported in the chapter on atherosclerosis, it is reasonable to conclude that CD49d is another gene, which shows GABP transefficiency-mediated suppression.

4. Technical Note: Cell Motility

a) Model

(1) Functions: Signal Intensity, Adhesion and Velocity

(a) Model: Skewed-Bell

The skewed-bell model of cell motility describes the relation between signal intensity, adhesion, and velocity.

Let [Signalᵢ] denote the intensity of Signalᵢ. Consider a range Q of intensities. The skewed-bell model of cell motility is based on two premises:

(1) The relation between [Signalᵢ] and adhesion of the cell to other cells, or the extracellular matrix, denoted [Adhesion], can be represented by an “increasing S-shaped” function over Q.

(2) The relation between [Adhesion] and cell velocity, V, can be represented by a “skewed to the right,” “bell-shaped” function (hence the name skewed-bell).

Consider the following numeric example. The example uses specific functions. However, a sensitivity analysis that varied the functions and recalculated the results verified the robustness of the prediction below (see Appendix).

A. Assume a certain range, Q, of signal intensities 0<[Signalᵢ]<1.

B. Assume the following S-shaped function represents the relation between [Adhesion] and [Signalᵢ].

$$[ Adhesion ] ( [ {Signal}_{i} ] ) = \frac{{a ( [ {Signal}_{i} ] )}^{S}}{{b}^{S} + {( [ {Signal}_{i} ] )}^{S}} \qquad Function 8$$

Call Function 8 the “adhesion function.” The table lists three possible sets of parameters for the adhesion function.

TABLE 2
Three set of parameters for the adhesion function.

| Case | a | b | s |
|---|---|---|---|
| “slower increase” | 20 | 0.25 | 4 |
| ”lower increase” | | | |
| “faster increase” | 20 | 0.18 | 4 |
| “higher increase” | 40 | 0.25 | 4 |

FIG. 11 illustrates the values of the adhesion function calculated for the three cases over the defined range of signal intensities. The graphs are drawn to scale. A smaller “b” value results in a faster increase in adhesion (compare “slower increase” and “faster increase”). A larger “a” value results in higher increase in adhesion (compare “lower increase,” same as “slower increase,” and “higher increase”). Both changes result in an increase in the adhesion curve.

Note: A shift-up in the adhesion curve, or adhesion function, is different from an increase in adhesion. A shift-up in adhesion means a shift from the original curve to a new curve positioned left and up to the original curve. An increase in adhesion means movement on the original curve from low to high adhesion values (see more on this difference below). C. For economy of presentation, denote [Adhesion] with “y.” Assume the following skewed to the right, bell-shaped function, represents the relation between cell velocity, and adhesion, or between V and y.

$$V ( y ) = g \sqrt{\frac{ⅇ}{2 {ππ}^{3}}} exp ( - \frac{ⅇ}{2 y} {( \frac{y - f}{f} )}^{2} ) \qquad Function 9$$

Call Function 9 the “velocity function.” Assume e=2, f=3 and g=1 for all three cases.

Note: The current work assumes a skewed to the right, bell shape V function without attempting to derive it from concepts that are more fundamental. To complement the current work, one can consider DiMilla 1991²⁹³, which derived the skewed to the right, bell shape of the V function from an asymmetry between cell/substratum interactions at the lamellipod and uropod, or front and rear ends of the moving cell.

D. Insert Function 8 into Function 9. The new function represents the relation between V and [Signalᵢ].
V=f([Signalᵢ]) Function 10

The following graphs illustrate the values for Function 10 calculated for the three cases above.

A shift-up in adhesion from “slower” to “faster,” or from “lower” to “higher,” increases the skewness of the corresponding bell-shaped curves. For instance, the shift-up from “low” to “high,” increases skewness of the V curve from 2.0 to 2.3. Note that skewness greater than zero is defined as skewness to the right, and skewness less than zero, as skewness to the left. A shift-down in adhesion decreases skewness.

(b) Predictions and Observations

(i) Palecek 1997

A study (Palecek 1997²⁹⁴) measured cell-substratum adhesion and cell velocity at different substratum ligand levels, integrin expression levels, and integrin-ligand binding affinity. Integrin receptor expression was varied by selecting populations of CHO B2 cells with different relative expression levels of the integrin receptor α₅β₁ following transfection of the α₅-deficient CHO B2 cells with human α5 cDNA. The study varied integrin affinity by transfecting CHO cells with the lower (α_{IIb}β₃) or higher affinity (α_{IIb}β₃(β₁₋₂)) integrin receptor. To measure cell velocity, the study incubated the transfected cells on coverslips coated with fibronectin, the ligand for the α₅β₁, and α_{IIb}β₃ integrin receptors. Real-time digital image processing was used to acquire images and calculate cell centroid position asia function of time. Five to ten cells per field in 10 fields were scanned every 15 minutes for 12 hours. The digitized images were reviewed and the position of up to 20 cells was determined on each image, producing a (x, y) record of cell position. For each cell the squared displacement, D²(t), was calculated for every possible time interval. The persistence time (P), and random motility coefficient (μ) were calculated by regression to produce a best fit in a commonly used model of cell migration: D²(t)=4μ(t−P(1+e^{-t/P})) (details of the model are available in Parkhurst 1992²⁹⁵). In three dimensions μ=S²P/3 where S is the average speed of the migrating cells. To measure adhesion, the study incubated transfected CHO cells on fibronectin coated glass slides for 20 minutes. The cells were detached by placing the slides in a shear-stress flow chamber under flow of PBS with Ca⁺² and Mg⁺². Cells were counted before and after flow detachment in 20 fields along the slide, and the results were used to calculate the mean detachment force.

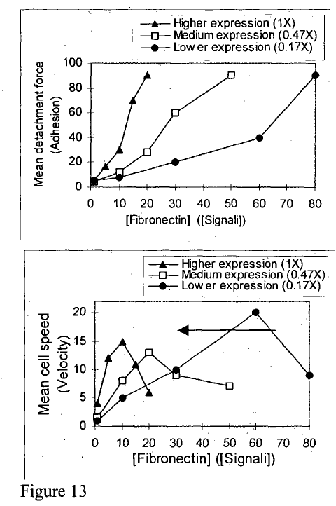

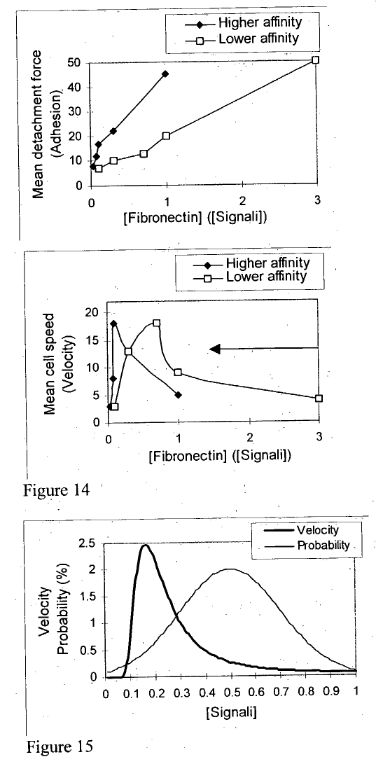

Consider fibronectin as the signal and coating concentration as signal intensity. According to the skewed-bell model of cell motility, an increase in fibronectin coating concentration should result in an S-shape increase in adhesion, and a skewed to the right, bell shape increase in velocity. Moreover, an increase in integrin receptor concentration or affinity should shift-up the adhesion curves and increase the skewness of the velocity curves. FIG. 13 and FIG. 14 summarize the observations reported in Palecek 1997 (ibid).

Compare the figures summarizing the observations and the figures illustrating the model. Although the study reports a small number of observations, the results are consistent with the skewed-bell model of cell motility. According to Palecek, et al., (1997, ibid) maximum cell migration speed decreases with an increase in integrin expression, or increase in integrin-ligand affinity. Moreover, “the maximum speed attainable . . . remains unchanged as ligand concentration, integrin expression, or integrin-ligand affinity vary.” Both conclusions are consistent with the increase in skewness. To explain the mechanism underlying the decrease in cell velocity at high adhesion levels, Palecek, et al., suggested: “high cell-substratum adhesiveness probably hinders cell migration by obstructing the release of adhesion at the rear of the cell.” (On the integrin dynamics of the tail region, see also Palecek 1998²⁹⁶, Palecek 1996²⁹⁷. For recent reviews discussing the study above and related observations, see Friedl 2001²⁹⁸, and Holly 2000²⁹⁹).

(ii) Bienvenu 1994

A study (Bienvenu 1994³⁰⁰) measured migration velocity of 100 leukocytes in the rat mesenteric interstitium, in vivo, using intravital videomicroscopy following exposure of the mesentery to 15 nM leukotriene B4 (LTB4).

The above presentation of the skewed-bell model of cell motility provides a description of the behavior of a single cell. The following section generalizes the model to the behavior of a population of many cells.

Assume a treatment with an agent of N₀ cells resulting in a normal distribution of Signalᵢ intensities. Let (μ, SD) denote the mean and standard deviation of the normal distribution. Let the probability of observing a certain velocity be equal to the probability density of the corresponding signal intensity. Consider the following numeric example.

A. Take an adhesion function with parameters: a=8.5, b=0.5, s=2, and velocity function with parameters: e=2, f=3, g=5.

B. Let (μ, SD) (0.5, 0.2), and N₀=100.

FIG. 15 presents the calculated velocities and distribution of signal intensities corresponding to the [0,1] range of signal intensities.

Consider signal intensity of 0.35. The corresponding velocity is 0.67912. The probability of observing a cell with such a signal intensity, and therefore such a velocity, is

$$1.5 \% ( P ( \frac{0.35 - 0.5}{0.2} ) = 1.50569 ) .$$

Since N₀=100, about 2 cells (100×0.150569˜2), or 1.5% of the cells should show velocity of 0.67912 (see figure below).

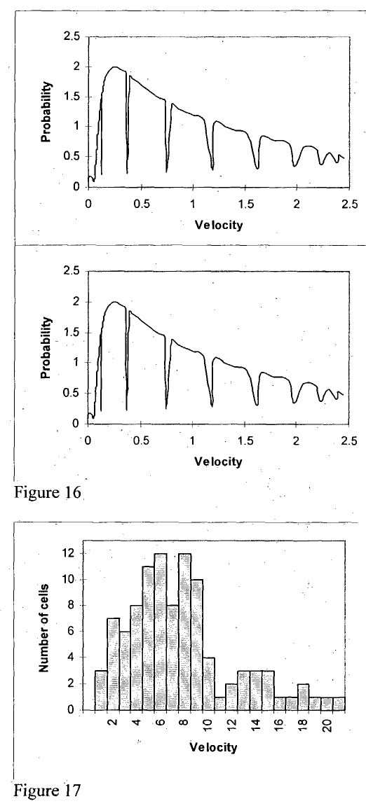

FIG. 16 represents the probability of observing all velocities corresponding to the [0,1] range of signal intensities according to the numeric example. The velocities are sorted from low to high. FIG. 17 presents the observed distribution of migration velocities (Bienvenu 1994, ibid, FIG. 2) (velocity is measured in μm/min).

Exposure to N-formylmethionyl-leucyl-phenylalanine (fMLP), platelet-activating factor (PAF), or ischemia-reperfusion (I-R), produced similar results (Bienvenu 1994, ibid, FIGS. 1, 3, 4)

The shape of the curve summarizing the observed velocities is similar to the shape of the curve summarizing the calculated velocities. The results are consistent with the skewed-bell model of cell motility.

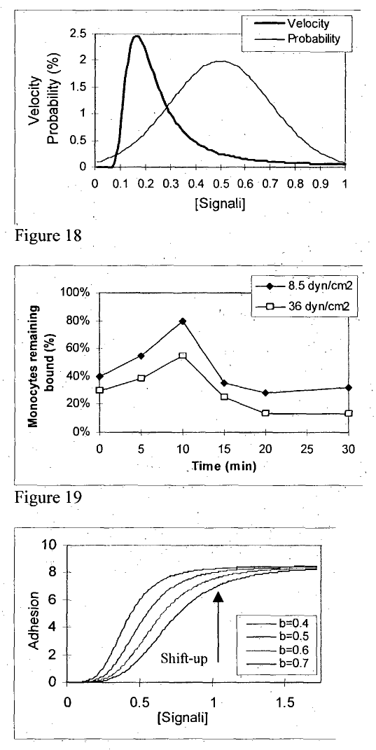

What is the source of the dips in the distribution curve? Consider FIG. 18. Points A and B have the same velocity value (0.67912, see above). Hence, a sort operation on velocities positions the points next to each other. However, the probability of point A (1.5%, see above) is larger than the probability of point B (0.28%). The difference in probabilities results from the velocity curve being skewed to the right, and the probability curve having a mean at the center of the range. As a result, the sort operation positions a velocity value with high probability next to a velocity value with low probability. The low probability in the middle of a group (continuum) of high probabilities creates the dips in the figure. Moreover, for the same velocity (for instance, A and B in the figure), the slope of the right side of the velocity curve is smaller than the slope of the left side (a characteristic of a skewed to the right, but not symmetrical bell shaped curve). Hence, the “number of points” (or density) of a range of velocities is larger on the right compared to the left side of the velocity curve. As a result, the “number” (or density) of velocities with higher probability is larger than the “number” (or density) of velocities with lower probabilities. As expected, in both the theoretical and empirical figures, the dips are sharp and the high grounds are wide. The shapes of the curve summarizing the observed velocities and the curve summarizing the calculated velocities are similar. Specifically, the number, position, and shape of the dips and high grounds are similar. The results are consistent with the skewed-bell model of cell motility.

(iii) Weber 1998, Weber 1996

A study (Weber 1998³⁰¹) stimulated 30 monocytes for 30 minutes with MCP-1 and measured random velocity on VCAM-1 during the 0-6.99, 7.0-13.99, 14.0-30.0 minute time intervals. To calculate velocity the study divided the lengths of individual cell paths, determined by adding up cell centroid displacement at every 1-min interval, by length of time. What is the expected distribution of the cell velocities according to the skewed-bell model of cell motility?

An earlier study by the same authors (Weber 1996³⁰²) measured the effect of MCP-1 stimulation on monocytes strength of adhesion to VCAM-1. Soluble VCAM-1 (10 μg/ml) was adsorbed on a plastic dish. The dish was assembled as the lower wall in a parallel wall flow chamber and mounted on the stage of an inverted phase-contrast microscope. The cells were prestimulated with MCP-1 (1 ng/ml) for the indicated periods after which 5×10⁵ cells per ml were perfused for 1 min through the flow chamber at 0.5 dyn/cm² to allow attachment. Shear was then increased in 10 s intervals, and the number of cells per field remaining bound at the end of each interval was determined. FIG. 19 presents the results (Weber 1996, ibid, FIG. 3C).

The average percent of monocyte remaining bound following 0-6.99, 7.0-13.99, and 14.0-30.0 minutes of MCP-1 stimulation is 51, 67, 31%, and 36,46, 16% for 8.5 and 36 dyn/cm², respectively. Consider a cell stimulated for 30 minutes. The results suggest that adhesion during the first 0-6.99 minutes and the last 14.0-30.0 minutes is lower than during the 7.0-13.99 minute interval.

Consider the following numeric example.

A. Take an adhesion function with parameters: a=8.5, b=0.7, s=4, and velocity function with parameters: e=2, f=3, g=50.

B. Let (μ, SD)=(0.5, 0.05), or (μ, SD)=(0.5, 0.1), and N₀=100.

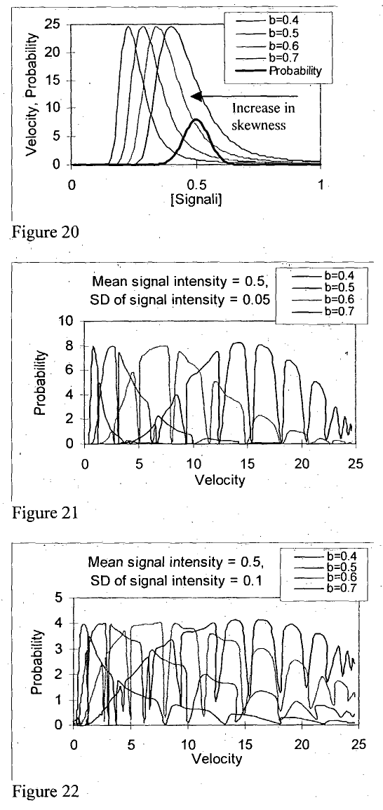

During the 7.0-13.99 minute interval, adhesion is higher than during the 0-6.99 minute interval. Consider the [0,1] range of signal intensities. The increase in adhesion between the two time intervals can be considered as an exogenous change in terms of the relation between signal intensity and adhesion (represented by the adhesion function). Therefore, the increase of adhesion over time can be represented as a shift to the left of the adhesion function, or an increase of adhesion for every level of signal intensity (see increase in skewness above). A shift-up of the adhesion curve increases the skewness of the velocity curve. In the numeric example, a shift-up in adhesion is presented as a decrease in the “b” parameter of the adhesion function. FIG. 20 presents the shift-up in adhesion, increase in skewness of velocity, and the probability of observing a certain velocity after sort for four “b” values.

FIG. 22 presents the probability of observing a certain velocity after sort for the same four “b” values but for a higher SD of signal intensity. Note the effect on the dips. The shape of the b=0.5 curve is similar to the shape of the calculated curve presented in the section describing the Bienvenu 1994 (ibid) study above.

In both cases, an increase in adhesion increased the skewness of the bell-shaped velocity curve.

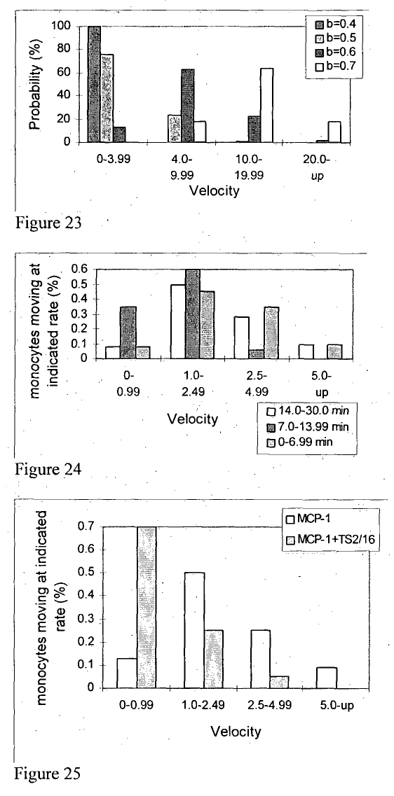

The Weber 1998 (ibid) study presents the results in histograms. The following intervals of cell velocities, 0-0.99, 1.0-2.49, 2.5-4.99, and 5.0-up, expressed in μm/min, define the bins. To better compare the calculated and observed distributions, bins with similar proportions were defined for the calculated velocities. FIG. 23 presents the distribution of cell velocity as histograms.

FIG. 24 presents the observed distribution of monocyte velocity (Weber 1998, ibid, FIG. 4). Technical note: In the Weber 1998 (ibid) study there is no gradient signal. Hence, for every time interval, the measured velocity is averaged around one point on the velocity figure, and therefore, provides an estimation of the instantaneous velocity. In a way, there is no time interval that represents an interval of signals; the signal is the same, randomly distributed around a certain signal.

The observed cell velocity distribution for the 7-13.77 minute interval, associated with higher adhesion, is positioned left of the distribution for the 0-6.99 minute interval. The calculated cell velocity distribution for the “b” value of 0.6, associated with higher adhesion, is also positioned left of the distribution for the “b” value of 0.7. Moreover, the shapes of the distributions are similar. The results are consistent with the skewed-bell model of cell motility, and specifically with the theoretical concept of increase in skewness. Moreover, note that the velocity distribution for the 14.0-30.0 minute interval, associated with lower adhesion, is positioned right of the distribution for the 7.0-13.99 minute interval. The result is consistent with the theoretical concept of decrease in skewness.

In another experiment, the same study measured random migration of monocytes on VCAM-1 in the presence of MCP-1 alone, or in combination with TS2/16, the β1 integrin affinity-activating mAb. FIG. 25 presents the results (Weber 1998, ibid, FIG. 2, B and E).

TS2/16 increases adhesion; therefore, it should increase the skewness of the velocity curve. As expected, addition of TS2/16 increased the skewness of the velocity curve. The results are consistent with the skewed-bell model of cell motility.

(2) Skewness and Velocity

(a) Model

Assume a given increase in skewness. Consider the point where the two velocity curves cross each other (see figure above). Call the signal intensity of that point “intensity of equal velocity.” In the numeric example, the intensity of equal velocity is about 0.1 for the shift from “lower” to “higher increase.” The intensity of equal velocity marks a turning point. At intensities lower than 0.1, cell velocity increased, and at intensities higher than 0.1, cell velocity decreased. In general terms, an increase in skewness increases cell velocity at all intensities less than the intensity of equal velocity, and decreases velocity at all intensities greater than the intensity of equal velocity.

A given increase in skewness increases velocity at low intensities and decreases velocity at high intensities.

Does the size of the increase in skewness influence the direction of change in cell velocity? In the (adhesion[Signalᵢ]) plane, a change in [Signalᵢ] will be called endogenous. A change in another variable will be called exogenous. An endogenous change corresponds to movement from one to another point on the same adhesion curve. An exogenous change corresponds to a shift of the curve. The effect of an exogenous change is mediated through a change in one or more of the “a,” “b” or “s” parameters.

Consider an exogenous change that decreases the “b” parameter. What is the effect of the exogenous change on cell velocity? Consider the following numeric example.

A. Assume an adhesion function with a=20, s=4.

B. Assume a velocity function with e=2, f=3, and g=1.

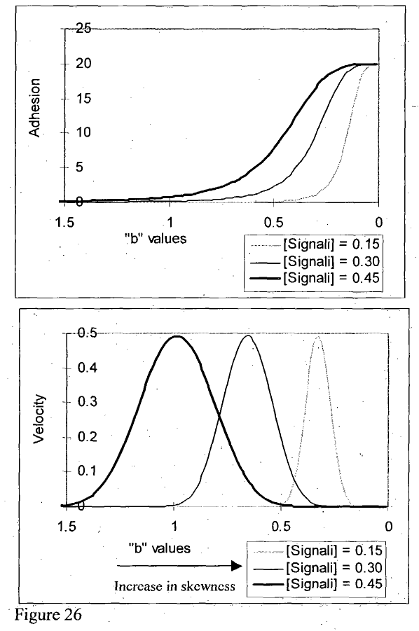

FIG. 26 presents adhesion and velocity as a function of “b” for three levels of signal intensity: 0.15, 0.30, and 0.45. Since an increase in “b” values decreases adhesion, the order of the “b” values on the x-axis is reversed.

Consider the velocity curve for [Signal₁]=0.45. An exogenous event, which decreases “b,” or increases skewness, first increases, and then decreases cell velocity. The same conclusion holds for the other two signal intensities. Examples of exogenous events that increase skewness are available below.

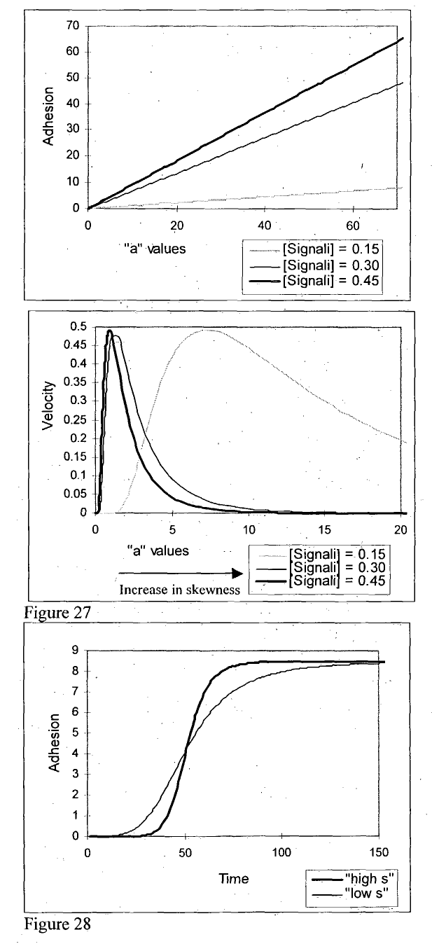

Assume an adhesion function with b=0.25, s=4. FIG. 27 presents adhesion and velocity as a function of the “a” parameter for the three signal intensities. The effect of a change in the “a” parameter is similar to a change in the “b” parameter. In both cases, an increase in skewness, first increases, and then decreases cell velocity.

An exogenous change mediated through a change in “s” values is different. An increase in S” pivots the adhesion curve; hence, it cannot be classified as a right- or shift-up. Consider FIG. 28.

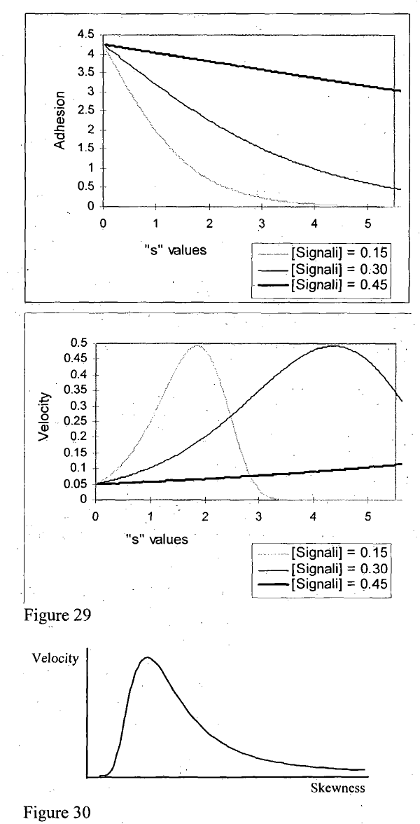

Nevertheless, assume an adhesion function with a 8.5 and b=0.5. FIG. 29 presents adhesion and velocity as function of “s” for the three signal intensities.

### SUMMARY

Consider Signals. An increase in adhesion exogenous to Signalᵢ, that is, an increase in adhesion with no change in Signalᵢ intensity, increases the skewness of the velocity curve with respect to Signalᵢ. In terms of the adhesion function, an increase in skewness corresponds to a decrease in “b,” increase in “a,” and decrease or increase in “s” depending on the Signalᵢ intensity.

According to the skewed-bell model of cell motility, for a given signal intensity (for instance, 0.45), an increase in skewness increases cell velocity of cells with low adhesion, and decreases cell velocity of cells with high adhesion (see arrows below the x-axis in the figures above). Moreover, small increase in skewness mostly maintains the direction of change in cell velocity, while large shifts do not. For example, consider a velocity left of the peak. A small increase in skewness increases velocity, and a somewhat larger increase in skewness increases velocity even further. However, a large increase in skewness might decrease cell velocity. FIG. 30 summarizes the relation between increase in skewness and velocity for a given Signalᵢ intensity.

(b) Predictions and Observations

(i) Weber 1998, Chigaev 2001

The Weber 1998 (ibid) study measured average monocyte velocity on VCAM-1 of controls and cells treated with MCP-1, a chemokine, TS2/16, a β1 integrin affinity-activating mAb, or with a combination of MCP-1 and TS2/16. The following table presents the results.

TABLE 3
Observed average monocyte velocity on VCAM-1 of
controls and cells treated with MCP-1, TS2/16,
or with a combination of MCP-1 and TS2/16.

| | | | | MCP-1 + |
|---|---|---|---|---|
| | Control | MCP-1 | TS2/16 | TS2/16 |
| Average | 0.89 ± 0.74 | 2.43 ± 1.36 | 0.31 ± 0.39 | 0.86 ± 0.82 |
| velocity | | | | |
| μm/min | | | | |

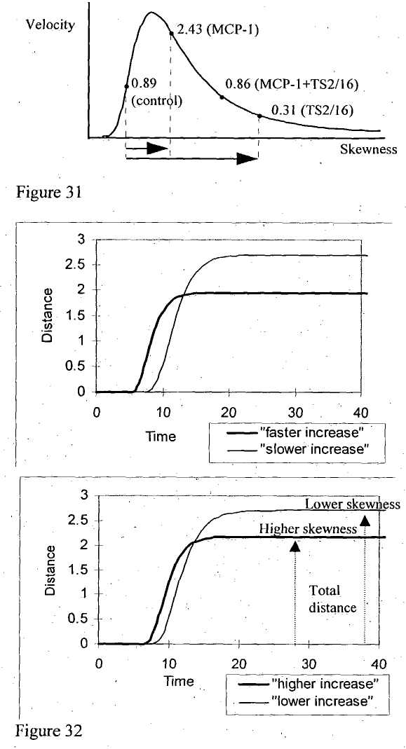

Place the observed velocities on the velocity/skewness curve (a higher velocity is placed higher on the curve). FIG. 31 presents the observations in Weber 1998 (ibid) in the context of the skewed-bell model of cell motility.

According to the figure, treatment with TS2/16 results in a larger increase in skewness relative to treatment with MCP-1. Since an exogenous increase in skewness is defined as an increase in adhesion for a given signal intensity, the figure suggests that treatment with TS2/16 should be associated with a higher adhesion level relative to MCP-1.

Another study (Chigaev 2001³⁰³) measured monocyte (U937) adhesion following treatment with TS2/16, Mn², fMLFF, or IL-5. The following table presents the K_{off}/10⁻⁴ of the treatment.

TABLE 4
Observed monocyte (U937) adhesion following
treatment with TS2/16, Mn², fMLFF, or IL-5.

| | | | | IL-5 | IL-5 |
|---|---|---|---|---|---|
| | TS2/16 | Mn²⁺ | fMLFF | (basophils) | (eosinophils) |
| K_{off}/10⁻⁴ | 19.0 | 13.0 | 100-210 | 100-150 | 130-230 |
| in s⁻¹ | | | | | |

Based on these results, Chigaev et al., (2001, ibid) concluded: “in all experiments we were able to detect the difference between the resting state and the activated state of α4-integrin. Moreover, dissociation rate constants were similar for all cells and all cell treatments (Table II), but dissociation rate constants in activated cells were at least 10 times greater than for Mn²⁺- or TS2/16-treated cells (Table I).” The study did not measure adhesion affinity following treatment with MCP-1. However, if we assume that MCP-1 induced affinity is similar to the tested chemoattractants, the study suggests that TS2/16 is, as expected, a more potent inducer of adhesion.

(3) Skewness and Distance

(a) Model

The first section below presents the relation between time and total distance traveled by a cell showing random motility. The second section extends the presentation to a cell showing directional motility.

(i) Random Motility

Assume a signal with an intensity that can be represented by an increasing S-shaped function of time. Since an increasing S-shaped function of an increasing S-shaped function is also an increasing S-shaped function, [Adhesion](t) and V(t) show the same shapes as the functions above. See the velocity/remoteness figure above.

Assume the following linear function represents the relation between [Signalᵢ] and t (linear function is a special case of an S-shaped function).
[Signalᵢ]=0.01 t Function 11

Call Function 11 the “signal function.” Insert Function 11 into Function 10 above. The new function represents the relation between V and t, that is, it defines V(t). The area under the V(t) curve represents the distance a cell traveled during the [0,t] time interval. FIG. 32 present the distance as a function of time for the four cases above.

(The shape of the adhesion, velocity, and distance functions is similar for “actual,” not linear, S-shape signal functions. Consider, for example, the following S-shape signal function:

$$[ {Signal}_{i} ] ( t ) = \frac{20 {t}^{3}}{{70}^{3} + {t}^{3}} \qquad Function 12$$

Note that the parameters of this S-shape signal function are the following: a=20, b 70, s=3).

Consider the points where the two distance curves cross each other (see figure above). Call the time of that point “time of equal distance.” In the numeric example, the time of equal distance is about 10 for the shift from “lower” to “higher increase.” The time of equal distance also marks a turning point. At times earlier than 10, distance increased, and at times later than 10, distance decreased. In general terms, an increase in skewness increases cell distance at all times earlier than the time of equal distance, and decreases distance at all times later than the time of equal distance.

Consider a time to where V(t₀)=0. According to the definition above,

$$TotalD = {\int }_{0}^{t0} V ( t ) ⅆ t \qquad Function 13$$

In FIG. 32, an increase in adhesion, or increase in skewness, decreased the total distance traveled by the cell. In the numeric example, both increases in skewness decreased total distance. From an initial distance of 2.71 for “slower increase”/“lower increase,” total distance decreased to 1.95 and 2.17 for “faster increase” and “higher increase,” respectively (see figure above). Decreased total forward distance results in a shorter stop (a stop closer to the starting point).

Technical Notes:

1. In the numeric examples, velocity never actually reaches zero. In the “faster increase” case, V(40)=2.53E-05. However, the “residual” velocity is so low (compare to V(8)=0.49), that it can be considered “rest.” To eliminate the residual velocity, a minimum velocity to support motility can be added to the velocity function. Such minimum velocity will decrease the residual velocity to zero.

2. Adhesion should be an S-shape function in the relevant range, defined as the range of the bell. Otherwise, in cases where adhesion is an accelerating function (the lower part of the S-shape), an increase in skewness will not produce the decline in area under the curve.

An increase in skewness is mediated through a decrease in “b,” increase in “a,” or change in “s.” What is the effect of a change in the size of the increase in skewness, or size of “b,” “a,” or “s,” on the distance traveled by the cell during a [0,t] time interval? Consider the following numeric example.
A. Assume the following signal function: [Signalᵢ]=0.01 t.
B. Assume an adhesion function with a=20, s=4.
C. Assume a velocity function with e=2, f=3, and g=1.

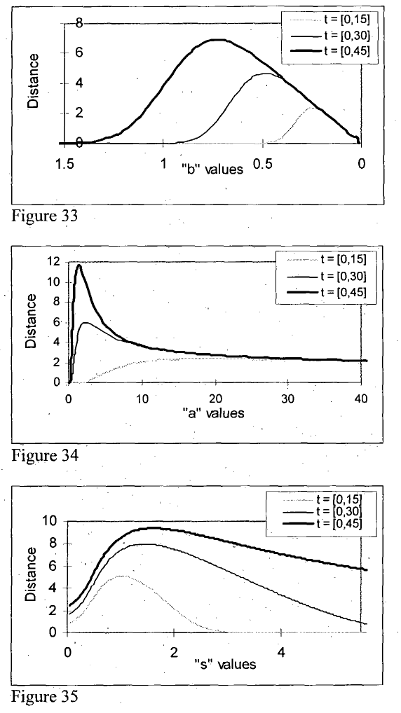

FIG. 33 presents distance as function of “b” for three time intervals [0,15], [0,30], and [0,45]. Since an increase in “b” values decreases adhesion, the order of the “b” values on the x-axis is reversed.

According to the t=[0,45] curve, an exogenous decrease in “b,” or increase in skewness of the Adhesion([Signal₁]) and Velocity([Signalᵢ]) curves, first increases, and then decreases the distance traveled by the cell during the given time interval. Same conclusion holds for the other two time intervals. Examples of exogenous events that increase skewness are available below.

Assume an adhesion function with b=0.25, s=4. FIG. 34 presents distance as function of “a” for the three time intervals. Similar to the effect of a decrease in the “b” parameter, an increase in “a,” or increase in skewness of the Adhesion([Signalᵢ]) and Velocity([Signalᵢ]) curves, first increases, and then mostly decreases the distance traveled by the cell during a given time interval.

An exogenous change mediated through “s” values is different. As mentioned above, an increase in “s” pivots the adhesion curve; hence, it is cannot be classified as shift-down or shift-up. Nevertheless, assume an adhesion function with a=8.5 and b 0.5. FIG. 35 presents distance as function of “s” for the three time intervals.

### SUMMARY

In many cases, the distance function takes the shape of an asymmetric bell, which indicates that, for a given time interval (say, [0,45]), an increase in skewness increases the distance a cell travels for cells With low adhesion, and decreases the distance for cells with high adhesion. Moreover, small increases in skewness mostly maintain the direction of change in distance, while large increases do not. For example, consider a distance left of the peak. A small increase in skewness increases the distance, and a somewhat larger increase in skewness increases distance even further. However, a large increase in skewness might decrease the distance.

(ii) Directional Motility

Consider an environment E. Take a reference point C in E. Denote the distance of a point x in E from C with Dist(x). Assume that every point in E is associated with certain Signalᵢ intensity. Signalᵢ will be called “gradient signal,” denoted Signal_{G}, if for all x0, x1 in E, such that Dist(x0)<Dist(x1), [Signal_{G}](x0)<[Signal_{G}](x1). An increase in the distance from C increases signal intensity.

Notes:

1. Assume that every tissue that supports cell motility produces a gradient signal. In haptotaxis, the molecule that produces the gradient signal can be bound to the extracellular matrix or cell surface (see examples below). Under such condition, a change in intensity of Signalᵢ, where Signalᵢ≠Signal_{G}, translates into a change in skewness of the velocity curve in the plane defined by the gradient signal.

2. A gradient signal changes random motility into directional motility.

3. The Palecek 1997 (ibid) study above measured random motility at different concentrations of fibronectin, each associated with different signal intensity. In each experiment, the study measured the average random motility of many cells and plotted the results as a single point on the velocity curve. The shape of the velocity curve was derived by “artificially” arranging the signal intensities associated with the different experiments in a “gradient” (represented by the x-axis in the figures which reported the results, see above). The actual experimental environments did not include a gradient signal.

4. For a gradient signal, the x-axis represents the actual environment, and the area under velocity curve, the directional distance traveled by the cell.

b) Excessive Skewness and Disease—an Example

A study (Cunningham 1986³⁰⁴) isolated polymorphonuclear leukocytes (PMN) from ten patients with chronic stable plaque psoriasis, five with more than 40%, five with less than 20% skin involvement, and ten healthy age- and sex-matched controls. The study measured the directional distance the cells migrated in agarose gel over a 2-hour period following stimulation with increasing concentrations of LTB4 or 12-HETE.

Leukotriene B4 (LTB4) produces a signal that increases CD18 mediated adhesion of polymorphonuclear leukocytes (PMN) to fibrin coated plates (Loike 2001³⁰⁵), mesangial cells (Brady 1990³⁰⁶), albumin-coated plastic surfaces, cultured human umbilical vein endothelial cells (HUVEC) (Lindstrom 1990³⁰⁷), and increases CD18 mediated adhesion of neutrophils to intercellular adhesion molecule 1 (ICAM-1) coated beads (Seo 2001³⁰⁸). Moreover, another study showed that high concentrations of the monoclonal 60.3, an antibody against CD18, inhibited PMN migration under agarose (Nilsson 1991³⁰⁹). Finally, a study showed that an antibody to CD18 decreased a 12-hydroxyeicosatetraenoic acid (12-HETE) induced neutrophil diapedesis (Fretland 1990³¹⁰). These observations suggest that LTB4 and 12-HETE increase CD18 mediated adhesion of PMN under agarose. Assume the increase in CD18 mediated adhesion is S-shaped. Then, according to the skewed-bell model of cell motility, the function that relates PMN velocity in agarose and LTB4 or 12-HETE concentrations should be skewed to the right, bell-shaped.

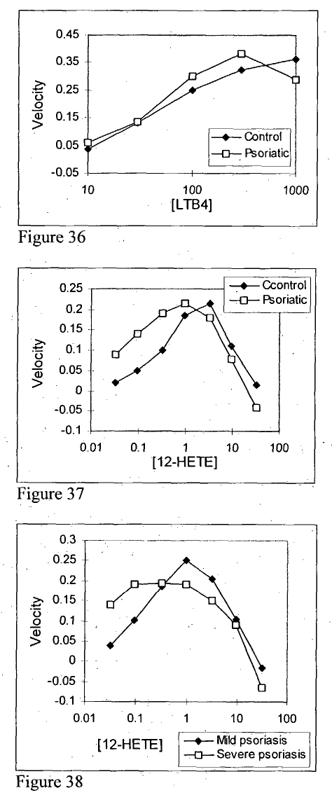

FIG. 36, FIG. 37, and FIG. 38 present the observed relations between PMN velocity and LTB4 or 12-HETE concentrations. The figures in the paper reported distances. To present velocities, the distances are divided by 2 hours, the migration time. Note that the x-axis is presented with a logarithmic scale (the figures are based on FIGS. 1, 2B and 3C in Cunningham 1986, ibid).

In mild vs. severe figure, peak velocity for severe patients seems to be lower than peak velocity for mild patients. However, the relatively large standard deviation of the peak for severe patients includes within its range the peak for mild patients.

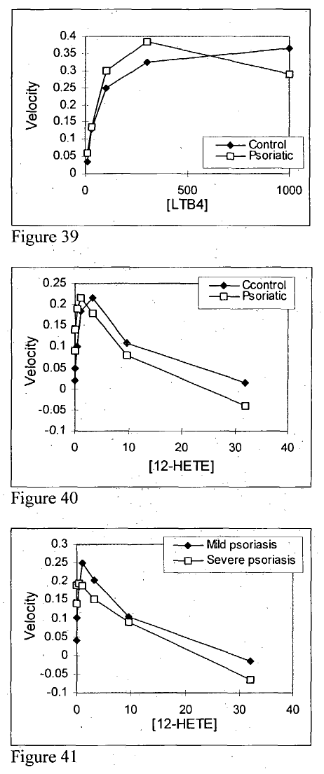

FIG. 39, FIG. 40, and FIG. 41 present the same observations with the x-axis in a linear scale. Note the right skewness of the bell-shaped curves.

As predicted, the functions that relate PMN velocity in agarose to LTB4 or 12-HETE concentrations is skewed to the right, bell shaped. Moreover, the observations suggest that psoriasis is associated with excessive skewness of the PMN velocity curve.

Notes:

1. Sun 1990 reported similar observations with PMN from psoriatic patients.

2. The chapter on atherosclerosis identifies a disruption that can cause the observed excessive skewness.

c) Appendix

All functions produce a velocity curve with the desired shape, that is, similar to the empirically derived shape.⁴
⁴ More information regarding these and other functions is available at http://www.causascientia.org/math_stat/Dists/Compendium.ndf.

Burr:

$$V ( y ) = \frac{GH}{F} {( \frac{y - E}{F} )}^{- G - 1} {( 1 + {( \frac{y - E}{F} )}^{- G} )}^{- H - 1} \qquad Function 14$$

Function 14 was inspired by the PDF of the Burr distribution. The Burr distribution, with H=1 is sometimes called Log Logistic or Fisk (see next function). FIG. 42 represents the results for “faster increase” vs. “slower increase” in adhesion (see above), where the velocity function is Function 14 with parameters (E,F,G,H)=(0,2,3,2).
Fisk:

$$V ( y ) = \frac{G}{F} {( \frac{y - E}{F} )}^{G - 1} {( 1 + {( \frac{y - E}{F} )}^{G} )}^{- 2} \qquad Function 15$$

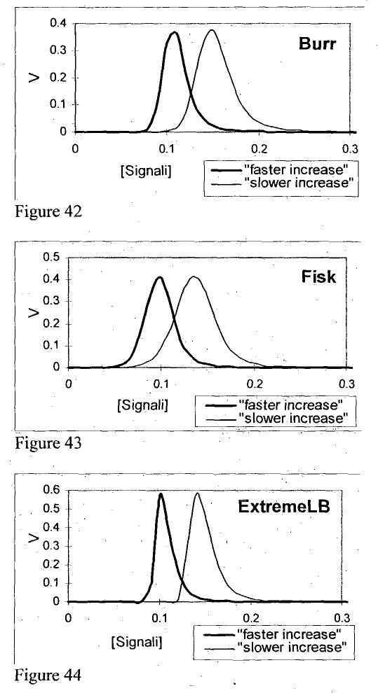

Function 15 was inspired by the PDF of the Fisk distribution. FIG. 43 represents the results for “faster increase” vs. “slower increase” in adhesion (see above), where the velocity function is Function 15 with parameters (E,F,G) (0,2,3).
ExtremeLB:

$$V ( y ) = \frac{G}{F} {( \frac{y - E}{F} )}^{- G - 1} exp ( - {( \frac{y - E}{F} )}^{- G} ) \qquad Function 16$$

Function 16 was inspired by the PDF of a typical extreme-value distribution with a lower bound. The corresponding distribution with an upper bound is Weibull(−x). FIG. 44 represents the results for “faster increase” vs. “slower increase” in adhesion (see above), where the velocity function is Function 16 with parameters (E,F,G)=(0.000001,2,3) (the “E” parameter is low since a condition of Function 16 is y>E).

5. Atherosclerosis

a) The Trucking Model of LDL Clearance

(1) LDL Pollution

Consider LDL in the intima as pollution. What is the source of the pollution? What are the dynamics of LDL pollution? Plasma LDL particles passively cross the endothelium (see observations in the passive influx section below). Unlike other tissues, the intima lacks lymphatic vessels. To reach the nearest lymphatic vessels, located in the medial layer, intimal LDL should cross the internal elastic lamina, an elastic layer situated between the intima and the media. However, “less than 15% of the LDL cholesteryl ester that entered the arterial intima penetrated beyond the internal elastic lamina” (Nordestgaard 1990³¹², see also Pentikainen 2000³¹³). A fraction of the LDL that entered the intima passively returns to circulation by crossing the endothelium (Bjornheden 1998³¹⁴, see also below). Another fraction is hydrolyzed. The remaining intimal LDL particles bind the intimal extracellular matrix (ECM). The ECM is composed of a tight negatively charged proteoglycan network. Certain sequences in the LDL apoB-100 contain clusters of the positively charged amino acids lysine and arginine. The sequences, called heparin-binding domains, interact with the negatively charged sulphate groups of the glycosaminoglycan chains of the proteoglycans (Boren 1998³¹⁵, Pentikainen 2000, ibid). Subendothelial agents modify (oxidize) the matrix bound LDL.

(a) Passive Influx

Nordestgaard 1992³¹⁶ reports a linear correlation between plasma concentration of cholesterol in LDL, IDL, VLDL and arterial influx. Moreover, in cholesterol-fed rabbits, pigs and humans, arterial influx of lipoproteins depended on lipoprotein particle size. Other studies reported independence of arterial influx of LDL in normal rabbits from endothelial LDL receptors. According to Nordestgaard 1992 (ibid), these results indicate that transfer of lipoprotein across endothelial cells and into the intima is a “nonspecific molecular sieving mechanism.” Schwenke 1997³¹⁷ measured intima-media permeability to LDL in different arterial regions in normal rabbits on a cholesterol-free chow diet. The results showed a 2.5-fold increase in permeability to LDL in the aortic arch compared to the descending thoracic aorta (Schwenke 1997, ibid, table 2). The concentration of undegraded LDL in the aortic arch was almost twice the concentration in the descending thoracic aorta (Schwenke 1997, ibid, table 3). Schwenke 1997 (ibid) also measured intima-media permeability to LDL in normal rabbits on a cholesterol-rich diet. The results showed similar intima-media permeability in all tested arterial regions compared to controls. The results also showed that the cholesterol-rich diet resulted in hypercholesterolemia and a substantial increase in transport of LDL cholesterol into all tested arterial regions (Schwenke 1997, ibid, table 2). Kao 1994³¹⁸ and Kao 1995³¹⁹ observed open junctions with gap width of 30-450 nm between adjacent endothelial cells in the breached regions' of the aortic arch. Unlike the aortic arch, the unbranched regions of the thoracic aorta showed no open junctions with such width. Moreover, the study observed LDL particles labeled with colloidal gold within most of the open junctions in the aortic arch, and no gold particles in normal intercellular channels (i.e., 25 nm and less) in both regions. These results are consistent with a nonspecific molecular sieving mechanism.

(b) Passive Efflux

Rabbits of the St Thomas's Hospital strain show elevated plasma levels of VLDL, IDL, and LDL. In aortic arches of these rabbits, in areas both with and without lesions, the logarithms of the fractional loss of VLDL, IDL, LDL, HDL, were inversely and linearly correlated with the diameter of the macromolecules (Nordestgaard 1995³²⁰). The observation suggests that, similar to influx, the efflux of LDL through the endothelium can also be described as a “nonspecific molecular sieving mechanism.”

(c) Summary

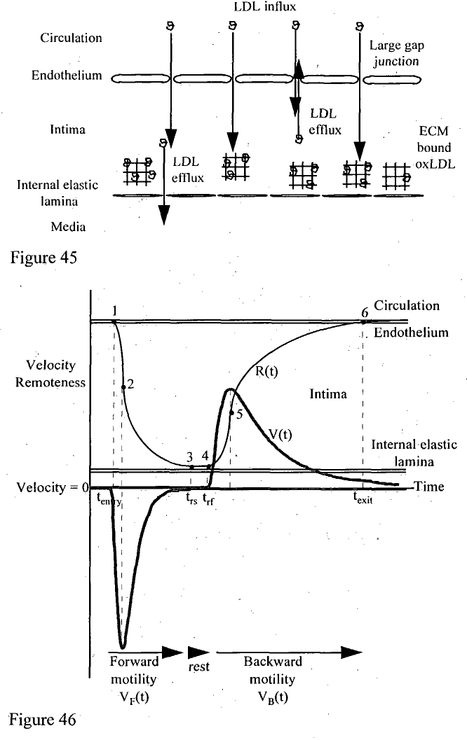

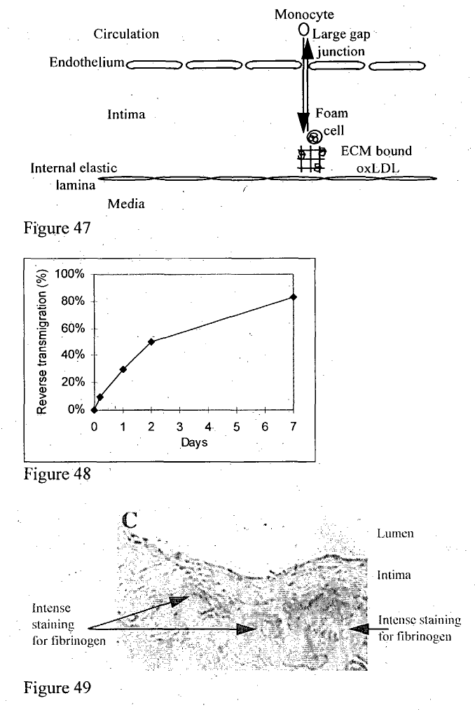

FIG. 45 illustrates the dynamics of LDL pollution in the intima.

Define “intimal LDL efflux” as the sum of LDL efflux through the endothelium and LDL efflux through the internal elastic lamina. Define “LDL retention” as the difference between intimal LDL influx and intimal LDL efflux. Then,
[oxLDL bound to intimal ECM] f(LDL retention) Function 17

Note: A fat-rich diet increases intimal LDL influx and intimal LDL efflux. However, intimal LDL efflux is only a fraction of intimal LDL influx. Therefore, a fat-rich diet increases LDL retention in the intima, which increases the concentration of oxLDL bound to the ECM.

(2) LDL clearance

(a) Conceptual Building Blocks

The extracellular matrix (ECM) is a stable complex of macromolecules surrounding cells. The matrix consists of two classes of macromolecules: glycosaminoglycans and fibrous proteins. Glycosaminoglycans are polysaccharide chains mostly found linked to proteins in the form of proteoglycans. Glycosaminoglycans form a highly hydrated, gel-like substance in which members of the fibrous proteins are embedded. Fibrous proteins include structural molecules, such as collagen and elastin, and adhesive molecules, such as fibronectin and laminin. Collagen fibers strengthen and organize the matrix. Elastin fibers provide resilience. Cells bind the matrix through surface receptors, such as integrins, cadherins, immunoglobulins, selectins, and proteoglycans. Cadherins and selectins mostly promote cell-cell adhesion. Integrins and proteoglycans mostly promote cell-matrix binding. The matrix provides the framework for cell migration.

Migration occurs in cycles. A cycle starts with formation of clear “front-back” asymmetry with accumulation of actin and surface receptors at the front end of the cell. This phase is called polarization. Migration continues with protrusion of the plasma membrane from the front of the cell in a form of fine, tubular structures called filapodia, or broad, flat membrane sheets called lamellipodia. Next, the cell forms new cell-matrix points of contact, which stabilize the newly extended membrane and provide “grip” for the tractional forces required for cell movement. A migration cycle culminates with flux of intracellular organelles into the newly extended sections, and retraction, or detachment of the trailing edge. Completion of a migration cycle results in directional movement of the cell body (Sanserson 1999³²¹)

Cell migration is a change of position of the entire cell over time. Projection is a change in position of a part of cell periphery overtime. Both cell migration and cell projection are called cell motility. Direction of movement can be defined as a change in distance relative to a reference point in space. Let circulation define a reference point. Migration of cells out, or away from circulation, will be called forward motility. For instance, diapedesis of monocytes to enter the intima (also called migration, emigration or transmigration) is, therefore, forward motility.

Migration of macrophages deeper into the intima is also forward motility. Migration of cells toward, or into circulation will be called backward motility. Reverse transendothelial migration of foam cells, or foam cell egression, are examples of backward motility.

(b) Model. Trucking

Macrophage clear ECM bound LDL in the intima. To clear modified LDL, circulating monocytes pass the endothelium, differentiate into macrophages, accumulate modified LDL, turn into foam cells, and leave the intima carrying accumulated LDL back to circulation. This sequence of events will be called the trucking model of LDL clearance, and the cells performing LDL clearance will be called trucking cells (for instance, monocytes, macrophages, and macrophage-turned foam cells are trucking cells).

Many studies reported observations consistent with the following sequence of quantitative events.
↑[oxLDL]_{ECM in intima}→↑[monocytes]ᵢₙₜᵢₘₐ →↑[macrophages]_{intima →↑[macrophage-turned foam cells]}ᵢₙₜᵢₘₐ
Sequence of quantitative events 2: Predicted effect of oxLDL in the intima on number of macrophage-turned foam cells in the intima.

On some aspects of this sequence, see two reviews: Kita 2001³²² and Valente 1992³²³ However, only a few studies documented the return of foam cells to circulation. Consider the following examples.

A study (Gerrity 1981³²⁴) fed a high fat diet to 22 Yorkshire pigs. The animals were killed 12, 15 and 30 weeks after diet initiation, and tissue samples were examined by light and electron microscopy. At 15 weeks, lesions were visible as raised ridges even at low magnification (Gerrity 1981, ibid, FIG. 1). Large numbers of monocytes were adherent to the endothelium over lesions, generally in groups (Gerrity 1981, ibid, FIG. 5), unlike the diffused adhesion observed at pre-lesion areas. Foam cells overlaid lesions at all three stages, although more frequently at 12 and 15 weeks. The foam cells had numerous flap-like lamellipodia and globular substructure (Gerrity 1981, ibid, FIG. 6). Some foam cells were fixed while passing through the endothelium, trapped in endothelial junctions alone or in pairs (Gerrity 1981, ibid, FIG. 8, 9). In all cases, the attenuated endothelial cells were pushed luminally (ibid, FIG. 14). The lumenal portion of the trapped foam cells showed an irregular shape, with numerous cytoplasmic flaps (lamellipodia and veil structures), empty vacuoles and decreased lipid content compared to the intimal part of the cell (Gerrity 1981, ibid, FIG. 8, 9). Foam cells were also infrequently found in buffy coat preparations from arterial blood samples (Gerrity 1981, ibid, FIG. 7) and rarely in venous blood. According to Gerrity 1981, these findings are consistent with backward migration of foam cells, and suggest that such a migration indicates the existence of a foam cell mediated lipid clearance system.

Another study (Faggiotto 1984-I³²⁵, Faggiotto 1984-II³²⁶) fed 10 male pigtail monkeys an atherogenic diet and 4 monkeys a control diet. For 13 months, starting 12 days after diet initiation, at monthly intervals, animals were killed and tissue samples were examined by light and electron microscopy. The endothelial surface of the aorta in control animals was covered with a smooth, structurally intact endothelium (Faggiotto 1984-I, ibid, FIG. 4A). Occasionally, the surface showed small focal areas protruding into the lumen (Faggiotto 1984-I, ibid, FIG. 4B). Cross sectional examination of the protrusions revealed foam cells underlying the intact endothelium (Faggiotto 1984-I, ibid, FIG. 3A). During the first 3 months, the endothelium remained intact. However, on larger protrusions, the endothelium was extremely thin and highly deformed. At 3 months, the arterial surface contained focal sites of endothelial separation with a foam cell filling the gap (Faggiotto 1984-I, ibid, FIG. 10A). The luminal section of the foam cell showed numerous lamellipodia. In addition, thin sections of endothelium cells bridged over the exposed foam cell, deforming the surface of the foam cell (Faggiotto 1984-I, ibid, FIG. 10B). Moreover, rare occasional foam cells were observed in blood smears of some controls. During the first 3 months, when the endothelium was intact, the number of circulating foam cells increased (Faggiotto 1984-II, ibid, FIG. 10). Based on these observations, Faggiotto, et al., (1984, ibid) concluded that foam cells egress from the artery wall into the blood stream, confirming the conclusion in Gerrity 1981 (ibid).

A third study (Kling 1993³²⁷) fed 36 male New Zealand White rabbits a cholesterol-enriched diet and 37 rabbits a control diet. Both groups were exposed to electrical stimulation (ES) known to induce atherosclerotic lesions. The stimulation program lasted 1, 2, 3, 7, 14, or 28 days. At these intervals, tissue samples were collected, processed, and examined by transmission electron microscopy (TEM). After 1 day of ES, intimal macrophages of hypercholesterolemic rabbits showed loading of lipids (Kling 1993, ibid, FIG. 3b). These cells were often responsible for markedly stretching the overlying endothelial cells. After 2 days, foam cells were fixed while passing through endothelial junctions (Kling 1993, ibid, FIG. 8a). Neighboring endothelial cells were often pushed luminally, indicating outward movement of the macrophage (Kling 1993, ibid, FIG. 8a). The intact intimal portion of the foam cells, and the ruptured luminal portion also indicate outward movement. The ruptured luminal portion was often associated with platelets (Kling 1993, ibid, FIG. 8b,c). Under the prolonged influence of the atherogenic diet, emerging foam cells became more frequent. In all cases, the emerging foam cells migrated through endothelial junctions without damaging the endothelium. Based on these observations, Kling, et al., (1993, ibid) concluded: “similar to observations of Gerrity and Faggiotto, et al., we have electro microscopic evidence that the macrophages, loaded with lipid droplets, were capable of migrating back from the intima into the blood stream . . . thus ferrying lipid out of the vessel wall.”

(3) Trucking

(a) Introduction

FIG. 46 summarizes the motility of an LDL trucking cell in the intima according to the skewed-bell model.

The following sections discuss the elements of the skewed-bell model.

(b) Propulsion

An LDL trucking cell carries two propulsion systems, one moves the cell forward, and the other moves he cell backward. Let V_{F}(t) and V_{B}(t) denote cell velocity at time t produced by the forward and backward propulsion systems, respectively (the shape of the curves in the figure is explained below). Note that V_{F}(t) and V_{B}(t) are vectors with opposite signs.

Let V(t) denote net velocity (or velocity for short), V(t)=V_{F}(t)+V_{B}(t). Note that, if V(t)>0, or V_{F}(t)>V_{B}(t), the trucking cell moves forward, if V(t)=0, or V_{F}(t)=V_{B}(t), the trucking cell is at rest, and if V(t)<0, or V_{F}(t)<V_{B}(t), the trucking cell moves backward.

Denote remoteness from the endothelium at time t with R(t). Then,

$$R ( t ) = {\int }_{{t}_{entry}}^{t} V ( t ) ⅆ t . \qquad Function 18$$

Under fixed velocity V₀, the R(t) function decreases to R(t)=V₀×(t−t_{entry}). Under variable velocity, remoteness is equal to the area under the V(t) curve from t_{entry} to t.

(c) Separation

Consider the time interval between entry and exit, denoted [t_{entry},tₑₓᵢₜ]. There exists a time to in [t_{entry},tₑₓᵢₜ], such that:
for every t<t₀, V_{F}(t)≧V_{B}(t);
and for every time t>t₀, V_{F}(t)≦V_{B}(t).

This condition will be called separation. According to separation, from t_{entry} to t₀, the cell moves forward, and from t₀ to tₑₓᵢₜ, the cell moves backward. The figure above presents a special case of complete separation, where, for every t, if V_{F}(t)>0, then V_{B}(t)=0, and if V_{B}(t)>0, then V_{F}(t)=0. In complete separation, the periods of forward and backward propulsion are completely separated from each other. The intermediate period, when forward and backward propulsion cancel each other, or when both forward and backward propulsion equal zero, will be called the rest period. In the figure, the horizontal segment of the cell remoteness curve between points 3 and 4 represents the rest period. Let tᵣₛ (from “rest starts”) denote the beginning of the rest period, and t_{rf} (from “rest finished”), the end of the rest period.

Then, for every t≧t_{rf},

$$\begin{matrix}R ( t ) = {\int }_{{t}_{entry}}^{t} ( {V}_{F} ( t ) + {V}_{B} ( t ) ) ⅆ t \\ = {\int }_{{t}_{enry}}^{{t}_{rs}} {V}_{F} ( t ) ⅆ t + {\int }_{{t}_{rf}}^{t} {V}_{B} ( t ) ⅆ t .\end{matrix} \qquad Function 19$$

The condition permits the above separation of integrals. Let D_{F}(t) and D_{B}(t) denote forward and backward distance, respectively.

$${D}_{F} ( t ) = {\int }_{{t}_{entry}}^{t} {V}_{F} ( t ) ⅆ t and {D}_{B} ( t ) = {\int }_{{t}_{rf}}^{t} {V}_{B} ( t ) ⅆ t \qquad Function 20$$

D_{F}(t) represents the distance a cell travels from t_{entry} to t, called forward distance. Let TotalD_{F} denote total forward distance, and let it be equal to D_{F}(t) for t=tᵣₛ, that is, TotalD_{F} is the distance a cell travels between entry and rest. D_{B}(t) represents the distance a cell travels from t_{rf} to t, called backward distance. Let t_{d} (from “done”) denote the time of exit from intima (t_{d}=tₑₓᵢₜ), or a time tᵢ>t_{rf}, such that V_{B}(tᵢ)=0, that is, a time, after rest, where the cell shows no backward motility, that is, stopped moving backward (t_{d}=tᵢ), or t_{rf} if for every time t>t_{rf}, V_{B}(t)=0(t_{d}=t_{rf}). Note that t_{rf}≦t_{d}≦tₑₓᵢₜ. If t=t_{d}, D_{B}(t) will be called total backward distance, denoted TotalD_{B}.

(d) Coordination

Let gF and gB denote genes associated with forward and backward propulsion, respectively. Denote activity of the protein expressed by gF by A_{gF}. There exist gF, gB, such that for every to there is a later time, t₁>t₀, such that:

$$[ {mRNA}_{gB} ] ( {t}_{1} ) = f {( {A}_{gF} ( t ) ) .}_{( + )} \qquad Function 21$$

This condition will be called coordination. According to coordination, an increase in gF activity at time t₀, increases gB expression at a later time t₁. The same holds for a decrease in activity. Note that separation requires that t, is included in the [t_{rf},tₑₓᵢₜ] time interval (during times earlier than t_{rf}, backward propulsion is zero, and therefore, cannot be decreased when gF activity decreases). The purpose of coordination is to prevent trucking cell trapping in the intima (see details below).

In terms of distances, a trucking cell modifies backward propulsion such that total backward distance is equal to total forward distance, that is, the cell induces backward propulsion at a level “just enough” for successful return to circulation. Symbolically,

$${TotalD}_{F } = {\int }_{{t}_{entry}}^{{t}_{r0}} V ( t ) ⅆ t = {\int }_{{t}_{rf}}^{{t}_{exit}} V ( t ) ⅆ t = {TotalD}_{B } . \qquad Function 22$$

Notes:

1. Coordination can also be represented as equal areas under the V(t) curve for the [t_{entry},tᵣₛ] and [t_{rf},tₑₓᵢₜ] time intervals.

2. A cell only moves in one dimension. A trucking cell does not turn, it reverses course (the shape of the cell remoteness curve in the figure above should not be confused with cell turning). Consider.

The following table compares propulsion in trucking cells and cars.

TABLE 5
Comparison between a trucking cell and car.

| | Trucking cell | Car |
|---|---|---|
| Number of propulsion | Two | One |
| systems | | |
| Type of change in | Reversing (forward- | Turning (circling, |
| direction | rest-backward) | continuous speed in |
| | | turn) |
| Space of all possible | One dimensional | Two dimensional |
| directions | (movement on a line) | (movement on a plane) |

(e) Summary

Consider the figure above. A point on the cell remoteness curve represents distance from the endothelium at a time t, and the slope of the tangent to the remoteness curve at that point equals velocity. Point 1 represents passing of the endothelium and entry into the intimal space. From point 1 to point 2, the forward directed velocity, and the slope of the remoteness curve, increases. From point 2 to point 3, the beginning of the rest period, tᵣₛ, the forward directed velocity, and the slope of the remoteness curve, decreases. During the rest period (point 3 to point 4, or tᵣₛ to t_{rf}), cell velocity equals zero, and remoteness is fixed. From point 4, the end of the rest period, t_{rf}, to point 5, backward directed velocity, and the slope of the remoteness curve, increases. From point 5 to point 6, the backward directed velocity, and the slope of the remoteness curve, decreases. Point 6 represents passing of the endothelium and exit from the intimal space.

(4) Propulsion Genes

(a) Genes and Propulsion

(i) CD18, CD49d Integrin and Forward Propulsion

(a) Adhesion

The integrins are a class of cell membrane glycoproteins formed as αβ heterodimers. There are 8 known α subunits (120 to 180 kD), and 14 P subunits (90 to 110 kD) (Hynes 1992³²⁸).

The β₂ leukocyte chain (CD18) forms three heterodimers: CD18/CD11a (LFA-1; Leu CAMa, β₂α_{L}), CD18/CD11b (CR3, Leu CAMb, Mac-1, Mol, OKM-1, β₂α_{L}), and CD18/CD11c (p150 (p150, 95) Leu CAMc, integrin β₂α_{X}). All three integrins are expressed on macrophages. Both CD18/CD11a and CD18/CD11b bind the intercellular adhesion molecule-1 (ICAM-1, major group rhinovirus receptor, CD54 antigen). Fibrinogen increases adhesion between CD18 heterodimers and ICAM-1 (Duperray 1997³²⁹, D'Souza 1996³³⁰, Languino 1995³³¹, Altieri 1995³³²)

The α₄ integrin (CD49d) forms two heterodimers: α₄β₁ (VLA-4, CD49d/CD29), and α₄β₇. Both α₄-heterodimers bind fibronectin and the vascular cell adhesion protein 1 (VCAM-1, CD106 antigen, INCAM-100).

(b) Motility

CD18-, and α₄-heterodimers propel forward motility. Several studies demonstrated a positive relation between expression of CD18 heterodimers, or VLA-4, and transendothelial migration (Shang 1998A³³³, Shang 1998B334, Meerschaert 1995³³⁵, Meerschaert 1994³³⁶, Chuluyan 1993³³⁷, Kavanaugh 1991³³⁸). The results in Shang 1998A (ibid), and Shang 1998B (ibid) also showed a positive relation between expression of CD18 heterodimers or VLA-4 and transmigration through a barrier of human synovial fibroblasts (HSF).

Another study (Fernandez-Segura 1996³³⁹) reports morphological observations that relate CD18 and forward motility. The study stimulated neutrophils with 10⁻⁸ M fMLP for 10 min. On unstimulated cells, CD18 was randomly distributed on the nonvillous planar cell body. Stimulation of the round, smooth neutrophils induced a front-tail polarity, i.e., a ruffled frontal pole and contracted rear end with a distinct tail knob at the posterior pole. Moreover, immunogold-labeling and backscattered electron microscopic images detected a 4-fold increase in CD18 surface membrane concentration compared to unstimulated cells. The immunogold-labeled CD18 accumulated mainly on ruffled plasma membrane at the frontal pole of polar neutrophils. The contracted rear end showed few colloidal gold particles. Based on these observations, Fernandez-Segura, et al., (1996, ibid), concluded that CD18 might participate in locomotion of neutrophils.

(ii) TF and Backward Propulsion

(a) Adhesion

TF binds the ECM through the plasminogen•fibronectin complex. See section on “Plasminogen and lipoprotein(a)” on page 200.

(b) Motility

Tissue factor (TF) propels backward motility. Consider the following observations.

(i) Morphological Observations

A study (Carson 1993³⁴⁰) showed preferential localization of TF antigen in membrane ruffles and peripheral pseudopods of endotoxin treated human glioblastoma cells (U87MG). Most prominent TF staining was observed along thin cytoplasmic extensions at the periphery of the cells. Moreover, membrane blebs, associated with cell migration, were also heavily stained. Another study (Lewis 1995³⁴¹) showed high concentrations of TF antigen in membrane ruffles and microvilli relative to smooth areas of the plasma membrane or endocytosis pits in endotoxin treated macrophages. The membrane ruffles and microvilli contained a delicate, three-dimensional network of short fibrin fibers and fibrin protofibrils decorated in a linear fashion with the anti fibrin (fibrinogen) antibodies. Treatment of macrophages with oxLDL resulted in similar preferential localization of TF antigen in membrane ruffles and microvilli.

Although the two studies use different terms, “cytoplasmic extensions” and “blebbed” (Carson 1993, ibid), and “microvilli” and “membrane ruffles” (Lewis 1995, ibid), the terms, most likely, describe the same phenomenon.

(ii) Cell Spreading

The human breast cancer cell line MCF-7 constitutively expresses TF on the cell surface. aMCF-7 is a subline of MCF-7. A study (Muller M 1999³⁴²) showed a significant increase in adhesion of aMCF-7 cells to surfaces coated with FVIIa or inactivated FVIIa (DEGR-FVIIa) during the first 2 h after seeding. In addition, the number of cells adhering to anti-TF IgG was significantly higher than the number of cells adhering to anti-FVII, or a control IgG (Muller M 1999, ibid, FIG. 6A). Accelerated adhesion and spreading of cells on surfaces coated with VIC7, an anti-TF antibody, was blocked by recombinant TF variants (sTF₁₋₂₁₉, sTF₉₇₋₂₁₉), which include TF residues 181-214, the epitope of the anti-TF antibody VIC7. No effect was seen with sTF₁₋₁₂₂. However, if anti-TF IIID8 (epitope area 1-25) was used for coating, sTF₁₋₁₂₂ blocked accelerated adhesion and spreading of cells. To conclude, Muller M 1999 (ibid) results demonstrate that, in vitro, cultured cells that constitutively express TF on the cell surface adhere and spread on surfaces coated with an immobilized, catalytically active, or inactive, ligand for TF.

Another study (Ott 1998³⁴³) showed that J82 bladder carcinoma cells, which constitutively express high levels of TF, adhere and spread on surfaces coated with an antibody specific for the extracellular domain of TF. The spontaneously transformed endothelial cell line ECV304, or human HUVEC-C endothelial cells, also adhere and spread on a TF ligand when stimulated with TNFα to induce TF expression.

In malignant and nonmalignant spreading epithelial cells, TF is localized at the cell surface in close proximity to, or in association with both actin and actin-binding proteins in lamellipodes and microspikes, at ruffled membrane areas, and at leading edges. Cellular TF expression, at highly dynamic membrane areas, suggests an association between TF and elements of the cytoskeleton (Muller M 1999, ibid). Cunningham 1992³⁴⁴ showed that cells deficient in actin binding protein 280 (ABP-280) have impaired cell motility. Transfection of ABP-280 in these cells restored translocational motility. Ott 1998 (ibid) identified ABP-280 as a ligand for the TF cytoplasmic domain and showed that ligation of the TF extracellular domain by FVIIa or anti-TF resulted in ligation of the TF cytoplasmic domain with ABP-280, reorganization of the subcortical actin network, and expression of specific adhesion contacts different from integrin mediated focal adhesions.

(iii) Reverse Transmigration

A study (Randolph 1998³⁴⁵) used HUVEC grown on reconstituted bovine type I collagen as an in vitro model of the endothelial-subendothelial space. The reverse transmigration assays used freshly isolated or pre-cultured peripheral blood mononuclear cells (PBMC) incubated with endothelium for 1 or 2 hours to allow accumulation of monocytes in the subendothelial collagen. Following initial incubation, non-migrated cells were removed by rinsing. At given intervals, the study processed a few cultures to enable counting of the cells underneath the endothelium. The remaining cultures were rinsed to remove cells that may have accumulated in the apical compartment by reverse transmigration, and incubation was continued. Let “reverse transmigration” represent the percentage decrease in number of cells beneath the endothelium relative to the number of subendothelial cells at 2 hours. FIG. 48 shows reverse transmigration as a function of time (Randolph 1998, ibid, FIG. 1A).

The figure shows that PBMC, which enter the subendothelial space, exit the culture by retransversing the endothelium with a t1/2 of 2 days. The endothelial monolayer remained intact throughout the experiments.

To examine the role of adhesion molecules in reverse transendothelial migration, the study treated cells with various antibodies. Two antibodies against TF, VIC7 and HTF-K108, strongly inhibited reverse transmigration for at least 48 hours (Randolph 1998, ibid, FIG. 2A). In comparison, 55 other isotype-matched antibodies, specifically, two antibodies against factor VIIa, IVE4 and IIH2, did not inhibit reverse transmigration (Randolph 1998, ibid, FIG. 2C). A direct comparison of the effect of VIC7 relative to IB4, an antibody against β2 integrin, revealed 78±15% inhibition of reverse transendothelial migration by VIC7 relative to no inhibition by IB4 in the same three experiments (Randolph 1998, ibid, FIG. 2B). None of the antibodies affected the total number of live cells in culture. Moreover, soluble TF inhibited reverse transmigration by 69±2% in eight independent experiments (Randolph 1998, ibid, FIG. 4).

Epitope mapping showed that the TF epitope for VIC7 included at least some amino acids between amino acids 181-214. Moreover, only fragments containing amino acid residues carboxyl to residue 202 blocked reverse transmigration effectively (Randolph 1998, ibid, FIG. 4). These observation indicate that TF amino acids 181-214 are essential for reverse transmigration

The study also observed TF mediated adhesion to the endothelium. Unstimulated HUVEC were added to wells coated with TF or control proteins in the presence or absence of an anti-TF antibody. After 2 hours of incubation, endothelial cell adhesion to TF fragments containing amino acid residues 202-219 was greater than adhesion to control surfaces, or to TF fragments lacking these residues (Randolph 1998, ibid, FIG. 8A). Spreading of HUVEC during the first 2 hours was observed on surfaces coated with TF fragments 97-219 or 1-219. Surfaces coated with TF fragments spanning amino acids 1-122 showed less spreading. These results indicate that endothelial cells express binding sites for TF, and that TF residues 202-219 participate in this adhesion.

(b) Propulsion Genes and Separation

In complete separation, for every t,
if V_{F}(t)>0, then V_{B}(t) 0, and if V_{B}(t)>0, then V_{F}(t) 0.

In other words, backward and forward propulsion are completely separated in time (see above).

(i) Prediction

In complete separation, at least one gene G is associated with backward propulsion, but not with forward propulsion. Since G in not associated with forward propulsion, inhibition of G should not change forward motility. The same conclusion holds for at least one gene “h” associated with forward motility. Inhibition of “h” should not change backward propulsion. Consider the following observations.

Note: To prove the existence of at least one gene G associated with backward propulsion, but not with forward propulsion, assume complete separation, and that all genes, which associate with backward propulsion also associate with forward propulsion. In particular, assume that G is associated with forward propulsion. When V_{B}(t₀)>0, expression of G is high. But since G is also associated with forward propulsion, high expression of G results in V_{F}(t₀)>0, contradicting the concept of complete separation.

(ii) Observations

Randolph 1998 (ibid) tested a variety of antibodies against several molecules known to mediate binding between leukocytes and endothelium during apical-to-basal transmigration. The antibodies showed access to the subendothelial antigens. However, as predicted, many of the antibodies, specifically, antibodies against vascular cell adhesion molecule-1 (VCAM-1), and platelet/endothelial cell adhesion molecule-1 (PECAM-1), showed no effect on reverse transmigration.

Randolph 1998 (ibid) also showed that antibodies against TF, which participates in backward motility, do not inhibit forward motility. Resting monocytes do not express TF. LPS stimulates the expression of TF on resting monocytes. The study showed that the anti-TF antibody VIC7 inhibits adhesion of LPS-stimulated, but not resting monocytes to HUVEC by 35±7%. However, VIC7 did not inhibit migration of LPS-stimulated monocytes already bound to the apical side of the endothelium. Since circulating monocytes, do not express TF, it is reasonable to conclude that TF does not participate in adhesion to the endothelium during forward motility (however, TF adhesion to the apical side of the endothelium is important in backward motility). Since TF also does not participate in the subsequent steps in apical-to-basal transendothelial migration, it is reasonable to conclude that TF does not propel forward motility.

Note: Ott, et al., (1998, ibid) noted that J82 cells spreading on a TF ligand showed a different morphology compared to cells adherent to fibronectin through integrins (Ott 1998, ibid, FIGS. 2A and 2B), which suggests a qualitative differences in the two adhesive events.

(c) Propulsion Genes and Coordination

(i) Prediction

According to coordination condition, there exist two genes, gF, gB, such that for every to, there is a later time, t₁>t₀, such that:

$$\begin{matrix}[ {mRNA}_{gB} ] ( {t}_{1} ) = & f ( {A}_{gF} ( t ) ) . \\ & ( + )\end{matrix} \qquad Function 23$$

An increase in activity of the gene gF, which propels forward motility at time t₀, increases expression of the gene gB, which propels backward motility at a later time t₁.

Let CD18 and CD49d integrin be two gF genes, and TF a gB gene. According to coordination, an increase in CD18 or CD49d integrin activity should increase TF expression at subsequent times. Consider the following observations.

(ii) Observations

Fan 1995³⁴⁶ showed that an anti-α₄, or anti-β₁ antibody, as a surrogate ligand, increases TF surface expression and mRNA in THP-1 monocytes. The study also showed increased nuclear translocation of the c-Rel/p65 heterodimer and activation of the NF-κB site in the TF promoter following binding of the antibodies to α₄ or β₁. Another study (McGilvray 1997³⁴⁷) also showed an increase in NF-κB translocation and TF expression following cross-linking of VLA-4 (α₄β₁, CD49d/CD29) by antibodies directed against α₄ or β₁.

McGilvray 1998³⁴⁸ showed a significant increase in procoagulant activity (PCA) and TF surface expression on purified monocytes following cross-linking of MAC-1 (CD18/CD11b) integrin by an anti-CD11b antibody (McGilvray 1998, ibid, FIG. 5). Fan 1991³⁴⁹ showed that an anti-CD18/CD11b antibody, as surrogate ligand, amplified the positive effect of LPS, or T-cell-derived cytokines, on cell surface expression of TF in human PBMC (Fan 1991, ibid, FIG. 6).

Marx 1998³⁵⁰ incubated mononuclear cells (MNCs) with VSMCs and ICAM-1-transfected Chinese hamster ovary (CHO) cells. Incubation of MNCs with VSMCs for 6 hours significantly increased PCA. Addition of anti-ICAM-1 antibodies dose-dependently inhibited the increase in PCA. Incubation of MNCs with VSMCs increased TF mRNA after 2 h, and TF protein concentration after 6 h. Incubation of purified monocytes with ICAM-1-transfected CHO cells significantly increased PCA compared to untransfected CHO cells. Anti-CD18, anti-CD11b, or anti-CD11c antibodies inhibited the increase in PCA. Based on these observations, Marx, et al., (1998, ibid) concluded: “Monocyte adhesion to VSMCs induces TF mRNA and protein expression and monocyte PCA, which is regulated by beta2-integrin-mediated monocyte adhesion to ICAM-1 on VSMCs.”

Note: Fibrinogen increases the affinity between CD18 and ICAM-1 (see above). As expected, a study (Lund 2001³⁵¹) showed that fibrinogen, dose-dependently, amplified an LPS-induced increase in tissue factor (TF) activity in monocytes.

(d) Propulsion Genes and Gradients

(i) Predictions

(a) ICAM-1 Forward Gradient

Let the following function represent the relation between intensity of Signal; and concentration of CD18/CD11a·ICAM-1.
[Signalᵢ] f([CD18/CD11a·ICAM-1]) Function 24

Assume the function “f” is an increasing S-shaped function of [CD18/CD11a·ICAM-1]. Assume a fixed concentration of CD18/CD11a on the surface of a trucking cell. Then, [ICAM-1] should produce a gradient signal in the intima, where ICAM-1 should show lowest concentration just under the endothelium, and highest concentration just above the internal elastic lamina. Call a gradient, which shows highest concentration near the internal elastic lamina, a forward gradient. Then, ICAM-1 should show a forward gradient.

According to the definition of gradient signal, Signalᵢ will be called “gradient signal,” if an increase in distance from a fixed reference point increases Signalᵢ intensity.

An increase in ICAM-1 concentration increases [CD18/CD11a·ICAM-1, which, according to “f,” increases [Signalᵢ]. Since CD18-heterodimers propel forward motility, ICAM-1 should show the lowest concentration at the beginning of the migration path, that is, just under the endothelium, and highest concentration at the end of the migration path, that is, just above the internal elastic lamina.

(b) Fibrinogen Forward Gradient

The biological function of fibrinogen is to increase adhesion between CD18/CD11a and ICAM-1 (see above). Therefore, under conditions that promote trucking cell forward migration, the intima should also show a forward fibrinogen gradient with the lowest concentration just under the endothelium and the highest concentration just above the internal elastic lamina.

(c) VCAM-1 Forward Gradient

VCAM-1 is a ligand for α₄-heterodimers, which also propel forward motility. Therefore, VCAM-1 should also show a forward gradient in the intima, that is, show the lowest concentration just under the endothelium, and the highest concentration just above the internal elastic lamina.

(d) Fibronectin Backward Gradient

Fibronectin is a ligand for TF. TF propels backward motility. Therefore, fibronectin should show a backward gradient in the intima, that is, lowest concentration just above the internal elastic lamina, and highest concentration just under the endothelium.

(ii) Observations

(a) Fibronectin Backward Gradient

As predicted, several studies showed a fibronectin gradient in the intima with the highest concentration just under the endothelium. See for instance, Jones 1997³⁵² (FIG. 3A-D). According to Jones, et al., (1997, ibid): “we show, for the first time in clinical tissue, that accumulation of Fn in the periendothelium is an early feature of pulmonary vascular disease that may favor SMC migration.” Moreover, “For Fn, an increase in its periendothelial distribution pattern was observed with disease progression and is consistent with the concept that Fn gradient promotes SMC migration from the media to the intima.” Another study (Tanouchi 1991³⁵³) showed a gradient of fibronectin in the intima of both control animals and cholesterol-fed male albino rabbits, with a “steeper” gradient in cholesterol-fed rabbits (Tanouchi 1991, ibid, table II, see details below). A third study (Shekhonin 1987³⁵⁴) observed “fibronectin in the extracellular matrix of aortic intima fatty streaks where it could be found immediately under the endothelium and diffusely scattered in the subendothelium” (Shekhonin 1987, ibid, FIG. 2a,b).

(b) Fibrinogen Forward Gradient

A study (Lou 1998³⁵⁵) fed wild-type mice an atherogenic diet for 2 months, then isolated the proximal sections of the aorta and stained the isolated sections for fibrinogen. FIG. 49 presents the results (Lou 1998, FIG. 1C) (fibrinogen staining in purple). The deep layers of the intima showed the most intense staining for fibrinogen. The superficial layers showed the least intense staining.

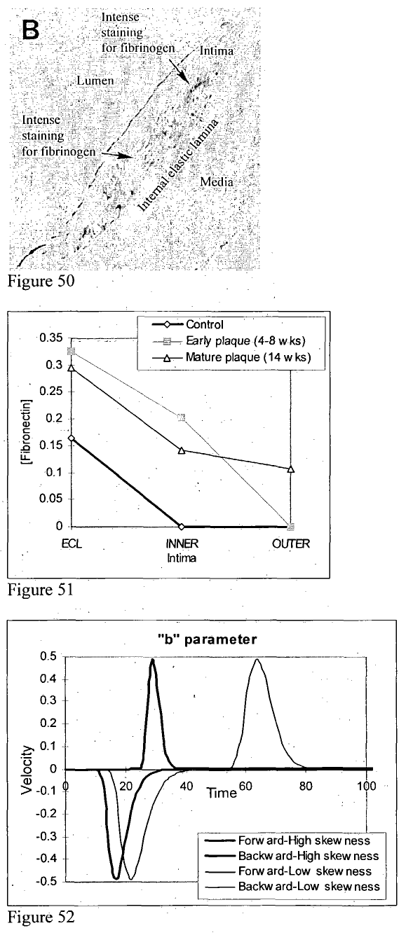

Another study (Xiao 1998³⁵⁶) stained sections from the proximal aorta of 22-week-old apoE(−/−)Fibrinogen(+/−) mice for fibrinogen. The sections showed fibrous lesions. FIG. 50 presents the results (Xiao 1998, ibid, FIG. 1B) (fibrinogen staining in red). Similar to Lou 1998 (ibid), the deep layers of the intima showed the most intense staining for fibrinogen, while the superficial layers showed the least intense staining.

The observations in Lou 1998 (ibid) and Xiao 1998 (ibid) are consistent with a forward fibrinogen gradient in the intima under conditions of LDL pollution.

(c) VCAM-1 Forward Gradient

A study (O'Brien 1993³⁵⁷) stained plaque in human coronary tissues for VCAM-1. Most staining was observed in SMC, and less commonly in macrophages and endothelial cells. The most intense staining was observed in; a subset of SMC positioned just above the internal elastic lamina, and in the upper layer of the media (O'Brien 1993, ibid, FIG. 2a,b,c). Some staining was also observed in macrophages and endothelial cells in areas of neovascularization in the base of plaques. The upper layer of the intima, just under the endothelium, showed no staining for VCAM-1.

Another study (Li 1993³⁵⁸) fed rabbits a cholesterol-rich diet for 13 weeks, isolated the atherosclerotic plaque, and stained the plaque for VCAM-1. Most intense staining was observed in a subset of SMC positioned just above the internal elastic lamina (Li 1993, ibid, FIG. 1A,B). The upper layer of the intima, just under the endothelium, showed no staining for VCAM-1.

The observations in O'Brien 1993 (ibid) and Li 1993 (ibid) are consistent with a forward VCAM-1 gradient in an intima under conditions of LDL pollution.

(iii) Comments

Assume a Signalᵢ, where Signalᵢ≠Signal_{G}. In addition, assume that Signalᵢ does not transform Signal_{G}. In the intima, ICAM-1, VSMC-1, and fibronectin show a signal gradient. The condition, therefore, assumes that Signalᵢ does not modify the concentrations of ICAM-1, VSMC-1, or fibronectin in the intima. Call such signal a “unit-transformation” signal (see explanation for the name below).

Assume that all functions except velocity are S-shaped. For instance, signal to mRNA, mRNA to surface concentration, surface concentration to adhesion, etc. Then, the function that relates signal to adhesion is also S-shaped. Consider the following sequence of quantitative events.
↑[Signalᵢ]→↑[mRNA_{CD18,α4, TF}]→↑[CD18, α₄, TF on cell surface]→↑Adhesion curve→↑Skewness of V_{F}, V_{B}
Sequence of quantitative events 3: Predicted effect of signal intensity on skewness of forward and backward velocity curves.

According to the sequence of quantitative events, the effect of a unit-transformation signal on cell migration can be presented as an increase or decrease in skewness of the forward or backward velocity curve.

Note: The unit-transformation condition can be relaxed. A monotonic transformation is a transformation that preserves the order, that is, “f” is monotonic, if for every xᵢ, xⱼ, such that xᵢ>xⱼ, f(xᵢ)>f(xⱼ). Define a unit-transformation as xᵢ=f(xᵢ). Then a unit-transformation is a special case of monotonic transformation. Call a signal that transforms the gradient monotonically, a monotonic signal. The effect of a monotonic signal on cell migration can also be presented as an increase or decrease in skewness of the forward or backward velocity curve.

A study (Tanouchi 1992, ibid) fed albino rabbits a high cholesterol-diet. At the end of the feeding period, the aorta was removed and stained for fibronectin. Staining intensity was quantified in three layers, endothelial layer (ECL), superficial area of the fatty streak plaque (INNER), and deep area of the fatty streak plaque (OUTER). presents the results (based on Tanouchi 1992, ibid, table II).

Note the backward fibronectin gradient. Also, note the monotonic transformations of the fibronectin gradient (see other examples for monotonic signals below).

b) Excessive Skewness and Atherosclerosis

(1) Model

(a) Excessive Skewness and Cell Depth

The following numeric example illustrates the relation between skewness and remoteness. The functions are the same as the ones found in the chapter on cell motility. In all cases assume [Signalᵢ]=0.0025t.

The table lists the sets of parameters for the CD18 and TF adhesion functions. Call the set “low skewness.”

Low Skewness

TABLE 6
Sets of parameters for the CD18 and TF adhesion
functions corresponding to low skewness.

| Adhesion function | a | b | s |
|---|---|---|---|
| CD18-forward motility | 29 | 0.13 | 4 |
| TF-backward motility | 30 | 0.22 | 11 |

The parameters for the velocity function for all cases are e=2, f=3, and g=1.

An increase in skewness can result from a decrease in the value of the “b” parameter or an increase in value of the “a” parameter. Consider first a decrease in the value of “b.”

(i) Decrease in “b” Parameter

The following table lists the sets of the new parameters for the CD18 and TF adhesion functions after the decrease in the level of “b.” Call the set “high skewness-“b” parameter.”

High Skewness-“b” Parameter

TABLE 7
Sets of parameters for the CD18 and TF adhesion functions
corresponding to low “b” mediated high skewness.

| Adhesion function | a | b | s |
|---|---|---|---|
| CD18-forward motility | 29 | 0.1 | 4 |
| TF-backward motility | 30 | 0.1 | 11 |

Note that the decrease in the value of the “b” parameter is proportionally larger for the TF adhesion function, a decrease of 55% and 23% for TF and CD18 relative to the “b” values of low skewness, respectively (see more on this point next).

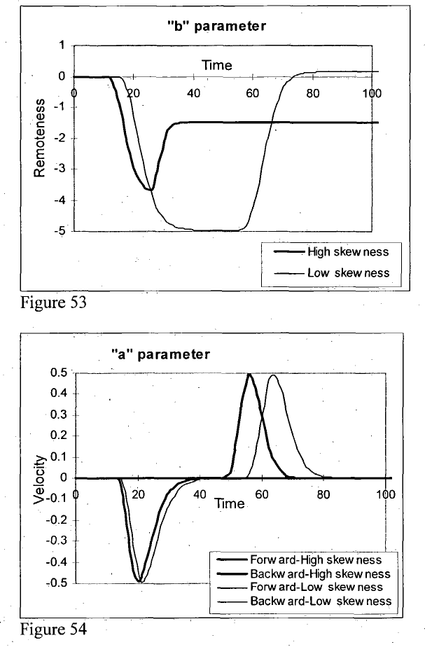

FIG. 52 and FIG. 53 present the velocity and remoteness curves for the two sets of parameters.

The arrows in the velocity figure point to the increase in skewness of the forward and backward velocity curves. The shape of the remoteness curve is similar to the one presented in the figure found in the trucking section above. Remoteness=0 illustrates the endothelium, and remoteness=−5, the internal elastic lamina. Notice the entry to the intimal space, the rest period, and the exit from the intimal space.

The increase in skewness decreases the maximum depth the trucking cell reaches, decreases the rest period, and prevents the cell from returning to circulation, or traps the cell in the intima.

(ii) Increase in “a” Parameter

The following table lists the sets of the new parameters for the CD18 and TF adhesion functions, after the increase in the level of “a.” Call the set “high skewness-“a” parameter.”

High Skewness-“a” Parameter

TABLE 8
Sets of parameters for the CD18 and TF adhesion functions
corresponding to high “a” mediated high skewness.

| Adhesion function | a | b | s |
|---|---|---|---|
| CD18-forward motility | 38 | 0.13 | 4 |
| TF-backward motility | 120 | 0.22 | 11 |

Note that the increase in the value of the “a” parameter is proportionally larger for the TF adhesion function, an increase of 300% and 31% for TF and CD18 relative to the “a” values of low skewness, respectively (see more on this point next).

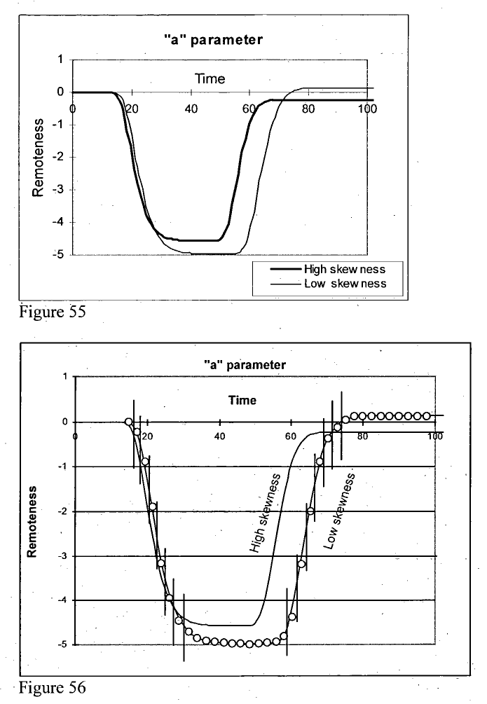

FIG. 54 and FIG. 55 present the velocity and remoteness curves for the two sets of parameters. The increase in the level of “a” also decreases maximum depth, decreases the rest period, and traps the cell in the intima.

Note that an exogenous event that shifts-up the CD18 or CD49d mediated adhesion curves, and increases the skewness of the forward velocity curve, produces a superficial stop. However, such an event does not trap the cell in the intima since TF expression is coordinated with CD18 expression, the increase in CD18 or CD49d expression increases TF expression. In contrast, an exogenous event that independently shifts-up the TF mediated adhesion curve, and increases the skewness of the backward velocity curve, traps the cell in the intima. Therefore, the following sections on tucking cell trapping center on TF expression. See further discussions on the difference between superficial stop and cell trapping in the section below entitled: “Excessive skewness, microcompetition, and atherosclerosis.”

Consider a study that stains the intima for macrophages. What will the staining show? Assume a uniform distribution over time of cell entry into the intima, that is, fixed time difference between cell entries to the intima, for instance, cell entry every 2 seconds. Consider FIG. 56.

A circle illustrates a cell. The horizontal distance between vertical lines illustrates the fixed time difference between cell entries to the intima. Table 9 presents the number of cells at certain depths in this figure. Round parenthesis indicates: “not including the border,” square parenthesis indicates: “including the border.”

TABLE 9
Number of cells at certain depths in the intima.

| Depth | Number of cells |
|---|---|
| [0, −1] | 7 |
| (−1, −2] | 2 |
| (−2, −3] | 0 |
| (−3, −4] | 3 |
| (−4, −5] | 14 |

For low skewness, maximum depth will show the most intense staining, and mid depth will show the least intense staining.

A similar analysis of the high skewness curve will show the most intense staining near the endothelium, at a superficial depth.

(b) Excessive Skewness and Lesion Formation

Consider FIG. 57 (see chapter on cell motility for the origin of the curve in the figure, specifically, FIG. 34, p158).

A point on the curve in the figure corresponds to an entire velocity curve in the plane defined by velocity and signal intensity. Each such point represents the velocity curve by its skewness and the area under the curve (see chapter on cell motility, p 145). The role of signal intensity is also different in the two planes. In the velocity-signal intensity plane, a point on the curve associates local signal intensity with cell velocity at that location. In the distance-a values plane, a point associates a gradient of signal intensities with the distance traveled by the cell in that gradient, at a given time interval.

Assume the skewness of the macrophage velocity curve is larger than the skewness of the SMC velocity curve in the same gradient (a gradient is a finite range of signal intensities arranged from smallest to largest, see chapter on cell motility, section on directional motility, p 158). There are many ways to formally present a difference in skewness (see chapter on cell motility, p 145). One possibility is to assume, for the two curves, the same “b” and “c” parameters, and a different “a” parameter. This possibility is consistent with observations in Thibault 2001³⁵⁹ (see FIG. 8), and Sixt 2001³⁶⁰ (see FIG. 4). Increased skewness is presented with a higher “a” value. Denote the difference in skewness with a₀, then,
a_{Mφ}=a_{SMC}+a₀, where a₀>0.

Increased skewness means that macrophages show peak velocity at a lower signal intensity compared to smooth muscle cells (see discussion, examples, and observations supporting this assumption in the section entitled “Angiotensin II and cell migration” below). The horizontal distance between corresponding Mφ and SMC points, such as Mφ₀, SMC₀, or Mφ₁ and SMC₁, marked with two arrows, is equal to the value of a₀. The value of a₀ can be described as the lag of the smooth muscle cells relative to macrophages.

Points Mφ₀, SMC₀ represent most efficient trucking. The gradient associated with Moo, SMC₀ supports the longest distance traveled by macrophages, which results in the smallest number of macrophages trapped in the intima. In the same gradient, smooth muscle cells show zero distance, and no migration into the intima. Points Mφ₀, SMC₀ also present the maximum rate of LDL clearance from the intima.

Points Mφ₁, SMC₁ represent excessive skewness of the macrophage and smooth muscle cell velocity curves. Excessive skewness results in a shorter distance traveled by macrophages, and a larger number of macrophages trapped in the intima. Excessive skewness also increases the distance traveled by smooth muscle cells, and the number of smooth muscle cells in the intima. Points Mφ₀, SMC₀ also present a decreased rate of LDL clearance, and therefore, accumulation of LDL in the intima. Accumulation of macrophages, smooth muscle cells, and LDL in the intima is the hallmark of atherosclerosis. Therefore, it is concluded that excessive skewness cause atherosclerosis.

(c) Skewness Moderation and Plaque Stability

Denote the number of SMC in and around plaque with [SMC]_{plaque}, and the number of macrophages trapped in and around the plaque [Mφ]_{plaque}. Then, plaque stability can be defined as a positive function of the ratio between [SMC]_{plaque} and [Mφ]_{plaque}. Symbolically,

$$Stability = f {( \frac{{[ SMC ]}_{plaque}}{{[ M ϕ ]}_{plaque}} ) .}_{( + )} \qquad Function 25$$

(i) Small Decrease in Skewness

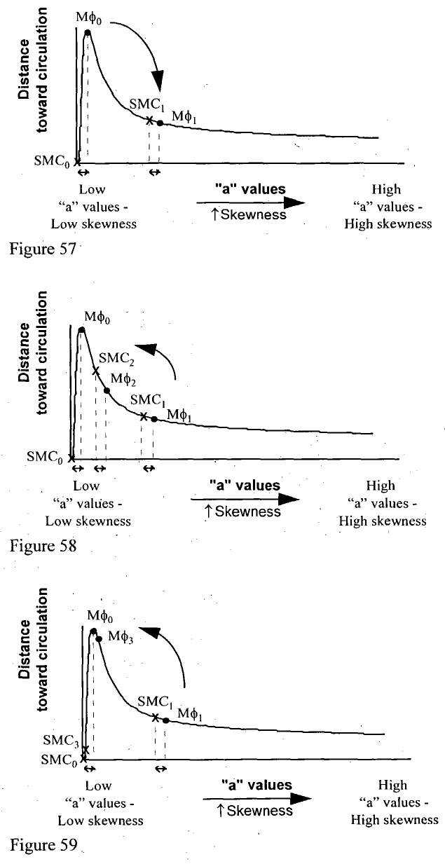

Assume a small decrease in skewness. Consider FIG. 58.

A decrease in skewness moves the points from Mφ₁, SMC₁ to Mφ₂, SMC₂. The new points indicate more smooth muscle cells and less macrophages in the intima. According to the definition, points Mφ₂, SMC₂ designate higher plaque stability.

Note: Increased stability does not correlate with lesion size. The decrease in the size of the lipid core, replaced by the increase in SMC (and collagen), can either increase, decrease or show no change in the lesion area, restenosis, etc., specifically, as measured by angiography.

(ii) Large Decrease in Skewness

Assume a large decrease in skewness. Consider FIG. 59. The large decrease in skewness moves the points from Mφ₁, SMC₁ to Mφ₃, SMC₃. The new points indicate little to no trapping of macrophages in the intima, and therefore, a sharp decrease in the number of macrophages in the intima. The points also indicate little or no entry of new smooth muscle cells to the intima. If, in this almost “healthy” situation, previously migrated SMC tend to undergo cell apoptosis, over time, the number of intimal SMC will decline.

Note: A large decrease in skewness also substantially decreases the lesion size.

(2) Predictions and Observations

(a) ApoAI and HDL

(i) Conceptual Background

Apolipoprotein AI (apoAI) is the main protein of high-density lipoprotein (HDL). Call cells in close proximity to an apoAI molecule, local cells. Lipid free apoAI, or HDL, stimulates cholesterol efflux from a variety of local lipid-loaded cells, such as, human skin fibroblasts, hepatocytes, smooth muscle cells, and macrophages. ApoAI and HDL are effective acceptors of plasma membrane cholesterol. However, studies also showed that apoAI stimulates translocation of cholesterol from intracellular compartments to the plasma membrane, and increases cholesterol efflux from intracellular compartment to serum (Tall 2002³⁶¹, von Eckardstein 2001³⁶², Rothblat 1999³⁶³, Phillips 1998³⁶⁴, Yokoyama 1998³⁶⁵).

The apoAI- and HDL-mediated increase in cholesterol efflux from local foam cells decreases the lipid content of such cells, specifically, the cellular concentration of oxLDL. Symbolically,
↑[ApoAI]OR ↑[HDL]→↓[oxLDL]_{local foam cell;}
Sequence of quantative events 4: Predicted effect of ApoAI of HDL in the intima on foam cell concentration of oxLDL.

Lipid free apoAI or HDL, near a Mφ-, or SMC-turned foam cell, decreases the concentration of oxidized LDL in the foam cell.

(ii) Predictions 1 and 2

(a) Prediction 1: Cell Depth

↑[ApoAI]ᵢₙₜᵢₘₐ OR ↑[HDL]ᵢₙₜᵢₘₐ ↓[oxLDL]_{local foam Mφ}. The decrease in concentration of oxLDL in the macrophage-turned foam cell shifts-down the CD18, α4, and TF adhesion curves, which decreases the skewness of the forward and backward velocity curves. Assume the effect on skewness of backward velocity curve is larger than the effect on forward velocity. The effect of a decrease in skewness of the macrophage velocity curves was analyzed in the section entitled “Excessive skewness, superficial stop, and cell trapping.” The analysis concluded that, under a condition of low skewness, maximum depth shows the most intense staining for macrophages, and mid depth, the least intense staining. Under a condition of high skewness, the region near the endothelium, at a superficial depth, shows the most intense staining. Therefore, the increase in apoAI, or HDL, should switch the intensive staining from a layer just under the endothelium to a layer deep in the intima. In non-lesion areas, the layer of most intense staining should be observed a little above the internal elastic lamina.

(b) Prediction 2: Plaque Stability

Consider the following sequence of quantitative events.

(i) Macrophages (Mφ)
↑[ApoAI]ᵢₙₜᵢₘₐ OR ↑[HDL]ᵢₙₜᵢₘₐ→↓[oxLDL]_{local foam MΦ}→↓[TF_{mRNA}]→↓TF_{MΦ} adhesion curve→↓Skewness of V_{B, MΦ} curve→↑TotalD_{B, M+101}→↓(TotalD_{F, MΦ}−TotalD_{B, M+101})→↓[MΦtrapped in intima] and ↓[LDL in intima]
Sequence of quantative events 5: Predicted effect of ApoAI of HDL in the intima on number of MΦ trapped in the intima, and LDL concentration in the intima.

An increase in local concentration of apoAI or HDL in the intima decreases the concentration of oxLDL in local lipid-loaded macrophages, decreases TF transcription in the macrophages, shifts-down the adhesion curve, decreases skewness of the backward velocity curve, and decreases the number of macrophages trapped in the intima.

(ii) Smooth Muscle Cells (SMC)

Small Effect

Assume a small effect of apoAI or HDL on [oxLDL] in local SMC. A small increase in concentration of apoAI or HDL in media and intima decreases the concentration of oxLDL in local lipid-loaded smooth muscle cells, decreases TF transcription in the SMC, shifts-down the SMC adhesion curve, decreases skewness of the velocity curve directed toward the intima, and increases the number of SMC in the intima.

Symbolically,
↑[ApoAI]_{intima/media} OR ↑[HDL]_{intima/media}→↓[oxLDL]_{local foam SMC}→↓[TF_{mRNA]→↓TF}_{SMC} adhesion curve→↓Skewness of V_{SMC} curve→↑TotalD_{SMC}→↑[SMC in intima]
Sequence of quantative events 6: Predicted effect of a small increase ApoAI or HDL in the intima/media on number of SMC in intima.

Note that the decrease in the number of macrophages and the increase in the number of SMC offset each other with respect to lesion area. Therefore, an increase in apoAI, or HDL, in the media and intima can increase, decrease, or cause no change in lesion area. However, if the initial event decreases lesion area, the change should be small. In terms of stability, an increase in intimal apoAI, or HDL, increases plaque stability.

Large Effect

Assume a large effect of apoAI or HDL on [oxLDL] in local SMC. Consider the following sequence of quantitative events. Two arrows denote large increase or decrease.
↑↑[ApoAI]_{intima/media} OR ↑↑[HDL]_{intima/media}→↓↓[oxLDL]_{local foam SMC}→↓↓[TF_{mRNA]→↓↓TF} _{SMC} adhesion curve→↓↓Skewness of V_{SMC} curve→↓TotalD_{SMC}→↓[SMC in intima]
Sequence of quantative events 7: Predicted effect of a large increase in ApoAI or HDL in the intima/media on number of SMS in intima.

A large increase in concentration of apoAI or HDL in media and intima decreases the concentration of oxLDL in local lipid-loaded smooth muscle cells, decreases TF transcription in the SMC, shifts-down the SMC adhesion curve, and decreases skewness of the velocity curve directed toward the intima. However, unlike a small decrease in skewness, a large decrease in skewness decreases the number of SMC in the intima (see figure above). A large decrease in skewness also substantially decreases lesion size.

Note that a “small” increase in apoAI or HDL concentration is defined as an increase in concentration that increases TotalD_{SMC}. In contrast, a “large” increase in apoAI or HDL concentration is defined as an increase in concentration that decreases TotalD_{SMC}. The size of the increase is defined by the effect on TotalD_{SMC}.

(iii) Observations

(a) Rong 2001

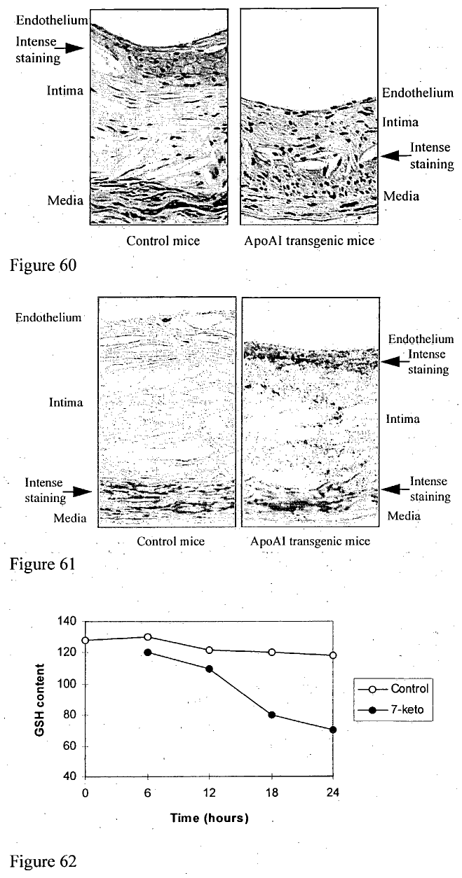

A study (Rong 2001³⁶⁶) fed apoE-deficient (EKO) mice a Western-type diet for 6 months. Then; segments of the thoracic aorta were removed and transplanted in the abdominal aorta of EKO mice expressing human apoAI in the liver (liver-AI), or control EKO mice not expressing the transgene. Prior to transplantation, both types of mice showed similar levels of non-HDL cholesterol. The liver-AI transgenic mice showed a higher level of HDL compared to controls (≈64 vs.≈26 mg/dL, respectively). Five months after transplantation, the grafts were analyzed. Staining with CD68, a macrophage specific marker, showed a significant decrease in, macrophage area in the intima of liver-AI transgenic mice compared to control (Rong 2001, ibid, FIG. 3B). Moreover, in controls, most intensive staining was observed just under the endothelium, while in liver-AI transgenic mice, the intense staining was observed deep in the intima, closer to the internal elastic lamina (Rong 2001, ibid, FIG. 3A). FIG. 60 shows exemplary grafts stained with CD68, a macrophage specific marker (brown) (from Rong 2001, ibid, fig; 3A). Magnification×100.

Staining with α-actin, a smooth muscle cell specific marker, showed a significant increase in SMC area in the intima of liver-AI transgenic mice compared to control (Rong 2001, ibid, FIG. 5B). Moreover, most intensive staining was observed just under the endothelium (Rong 2001, ibid, FIG. 5A). FIG. 61 shows exemplary grafts stained with α-actin, a smooth muscle cell specific marker (red) (from Rong 2001, ibid, FIG. 5A). Magnification×200.

The observations in Rong 2001 (ibid) are consistent with the predicted effect of a “small” decrease in skewness.

The study also measured lesion area. The following table summarizes the results. The liver-AI transgenic mice showed a smaller increase in lesion area. As predicted in the note above in the section on small decrease in skewness, the liver-AI transgenic mice showed a small change in lesion area, in the case of this study, small increase (compare these results to the results in the next studies).

TABLE 10
Observe lesion area in pre-transplanted, control transplanted,
and apoAI transgene transplanted mice.

| Mice | Lesion area (mm²) |
|---|---|
| Pre-transplanted (EKO mice) | 0.14 ± 0.04 |
| Control transplanted (EKO mice) | 0.39 ± 0.06# |
| ApoAI transgene transplanted mice | 0.24 ± 0.04* |
| (EKO + ApoAI) | |

#p < 00.1 compared to pre-transplanted
*p < 00.5 compared to control

Conclusion: High systemic concentration of human apoAI, expressed in the liver of transgenic mice, produces a “small” increase in apoAI concentration in the intima and media of the transgenic animals, where “smallness” is measured by the effect on skewness.

(b) Ishiguro 2001, Major 2001

A study (Ishiguro 2001³⁶⁷) produced transgenic mice expressing human apoAI (h-apoAI) under control of the macrophage-specific scavenger receptor-A promoter (Mφ-AI). The study then transplanted bone marrow from apoE(−/−), and apoE(−/−)Mφ-AI mice in liver-AI transgenic mice. Four weeks after transplantation, the mice were placed on a 16-week high-fat diet. The mean lesion area per section in the transplanted mice was seven times smaller in apoE(−/−)Mφ-AI compared to apoE(−/−) transplanted mice (58±21 vs. 424±208 μm², p=0.05). The two types of transplanted mice showed no difference in total cholesterol levels, or lipoprotein distribution. Production of apoAI by macrophages did change the levels of human apoAI or HDL in transplanted mice. Peritoneal macrophages from the apoE(−/−)M4-AI transplanted mice showed secretion of apoAI in culture medium, while macrophage from the apoE(−/−) transplanted mice showed no such secretion. Retroviral transduction instead of transgenic approaches produced similar results.

The observations in Ishiguro 2001 (ibid) are consistent with the predicted effect of a “large” decrease in skewness.

High systemic concentration in liver-AI mice produces a “small” increase in apoAI concentration in the intima and media of the transgenic animals. Expression of apoAI by intimal macrophages produces a “large” increase in local apoAI concentration. As mentioned above, “smallness” and “largeness” is measured by the effect on skewness.

A related study (Major 2001³⁶⁸) transplanted apoAI(−/−) mice with bone marrow from apoE(−/−) and apoE(−/−)Mφ-AI mice. Four weeks after transplantation, the mice were placed on a 16-week high-fat diet. In vitro analysis showed a more than 50% increase in cholesterol efflux from acLDL-loaded macrophages, in the presence of cyclodextrin (MBCD), in cells isolated from apoE(−/−)Mφ-AI compared to apoE(−/−) mice (p<0.05). Analysis of the lesion area in the transplanted mice showed a 96% decrease in lesion area in apoE(−/−)Mφ-AI compared to apoE(−/−) transplanted mice (p<0.05). The observations in Major 2001 are also consistent with the predicted effect of a “large” decrease in skewness.

(c) Duverger 1996

A study (Duverger 1996³⁶⁹) used transgenic rabbits expressing human apolipoprotein A-I in the liver. The transgenic rabbits and controls were fed a high-cholesterol diet for 14 weeks. Plasma levels of apo-B containing lipoproteins were similar in transgenic animals and controls. HDL levels in transgenic rabbits were about twice the levels of controls (68±11 vs. 37±3 mg/dL at week 14, p<0.001). To test cholesterol efflux, the study exposed Fu5AH cells to 5% diluted serum from transgenic rabbits and controls collected after the 14-week diet. Serum from transgenic rabbits increased cholesterol efflux significantly more than serum from controls (+24.5% of control at 2 hours, p<0.0001). Cholesterol efflux showed a correlation with total apoAI levels at 2 hours (p<0.005). Analysis of the thoracic aorta showed a 50% decrease in the percent of surface area covered with lesions in transgenic rabbits compared to controls (15±12 vs. 30±8, p<0.0027). Analysis of the abdominal aortas showed similar results.

(d) Plump 1994

A study (Plump 1994³⁷⁰) crossed transgenic mice, over expressing the h-apoAI gene in the liver (liver-AI), with apoE(−/−) mice. The apoE(−/−)liver-AI mice showed a significant increase in plasma HDL compared to apoE(−/−) mice (105±32 vs. 50±17 mg/dl, p<0.0001). The apoE(−/−)liver-AI mice also showed a significant decrease in lesion area compared to apoE(−/−) mice (at 4 months: 470±825 vs. 22,964±23,030 μm², p<0.0001, at 8 months: 45,222±35,631 vs. 243,200±202,698 μm², p<0.05).

(e) Shah 2001

A study (Shah 2001³⁷¹) administered a single injection of saline, 1080 mg/kg dipalmitoylphosphatidylcholine (DPPC), or 400 mg/kg of recombinant apoAI_{Milano} complexed with DPPC (1:2.7 weight ratio) to 26-week-old apoE(−/−) mice on a high cholesterol diet. One-hour post injection, plasma from apoAI_{Milano}-injected mice showed an almost 2-fold increase in their ability to induce cholesterol efflux from lipid-loaded cells compared to saline or DPPC injected mice (p<0.01). At 48 hours post injection, the aortic sinus showed a significant decrease in lipid and macrophage content in apoAI_{Milano} compared to saline and DPPC injected mice (lipid content: 10.1±4.2, 19.6±6.3, 18.1±4.7, % of plaque area, p<0.01 vs. saline and DPPC; macrophage content: 6.4±2.0, 10.4±3.4, 9.3±5.8, % of plaque area, p<0.01 vs. saline, apoAI_{Milano}, saline, and DPPC injected mice, respectively).

The observations in Duverger 1996 (ibid), Plump 1994 (ibid), and Shah 2001 (ibid) are consistent with the predicted effect of a decrease in skewness. However, it is not, clear from the observations reported in these studies whether the increase in apoAI concentration produced a “small” or “large” decrease in skewness. A measurement of additional resulting quantitative events, such as the number of SMC in the lesion, could have provided the answer.

(iv) Prediction 3: Infiltration vs. Egress

Assume that the main function of apoAI in the intima is to decrease skewness in cells “excessively” loaded with lipids. Consider an intima with no such cells. In this intima, an exogenous increase in apoAI will show no effect. Specifically, the increase in apoAI will show no effect on the extracellular concentration of lipids in an intima, or the number of monocytes recruited from circulation, or monocyte infiltration.

(v) Observations

(a) Dansky 1999

A study (Dansky 1999³⁷²) examined aortic sections of 6- to 8-week-old apoE(−/−) (E0) and apoE(−/−)liver-AI (E0/hA-I) transgenic mice. The intima from both E0 and E0/hA-I mice showed lipid associated with the extracellular matrix. E0/hA-I mice showed higher systemic concentrations of apoAI compared to E0 mice. However, as predicted, the number of areas containing lipid deposits, and the amount of lipid in the intima, were similar in both types of mice (Dansky 1999, ibid, table 1, table 2). In addition, as predicted, the staining areas for monocytes bound to the endothelium were similar in both types of mice (Dansky 1999, ibid, FIG. 7). Based on these observations, Dansky, et al., (1999, ibid) concluded: “Several hypotheses can be constructed to explain how the human apo A-I transgene dramatically attenuates foam cell formation despite the lack of an effect on lipid retention, endothelial activation, and monocyte adherence. . . . Third, elevated apo A-I and HDL-C could promote reverse cholesterol transport, decrease foam cell formation, and possibly promote macrophage egress from the vessel wall.”

Note: Most studies interpret an increase, or decrease in staining for macrophages in the intima as an increase, or decrease in monocyte infiltration to the intima. However, a change in staining can also result from a change in the number of macrophages returning to circulation, or cell egress. Therefore, readers of such studies are advised to reexamine the observations before adapting the authors' interpretation (see also the discussion in Dansky 1999, ibid, on the difference between staining of macrophages in the intima and rate of monocyte infiltration).

(b) Regression Diet

(i) Conceptual Background

(a) Oxidized LDL and Oxidative Stress

Minimally modified LDL (mmLDL) and oxidized LDL (oxLDL) deplete intracellular GSH, and therefore induce oxidative stress.

A study (Therond 2000³⁷³) determined the GSH content in cultured human endothelial cells after 24 h incubation with native LDL or oxLDL at 30, 40, and 50 μg of protein/ml. The results showed a 15 and 32% decrease of GSH content at 40 and 50 μg/ml (only significant at 50 Hg/ml, p<0.05), and a slight but significant increase (10%) of GSH content at 30 μg/mg (Therond 2000, ibid, FIG. 2B). The results also showed that all oxLDL lipid fractions depleted intracellular GSH (Therond 2000, ibid, FIG. 3B).

Another study (Lizard 1998³⁷⁴) tested the effect of a specific oxLDL fraction on intracellular GSH. Human promyelocytic leukemia cells, U937, were treated with 7-ketocholesterol. U937 respond to oxysterols in concentrations similar to the concentrations observed in endothelial and smooth muscle cells, and are frequently used to model the response of macrophages to oxysterols in humans. GSH content was measured by flow cytometry with monochlorobimane. FIG. 62 presents the results (Lizard 1998, ibid, FIG. 5A). The results showed lower GSH content in the 7-ketocholesterol treated cells compared to controls (p<0.05).

(b) Oxidative Stress and TF Transcription

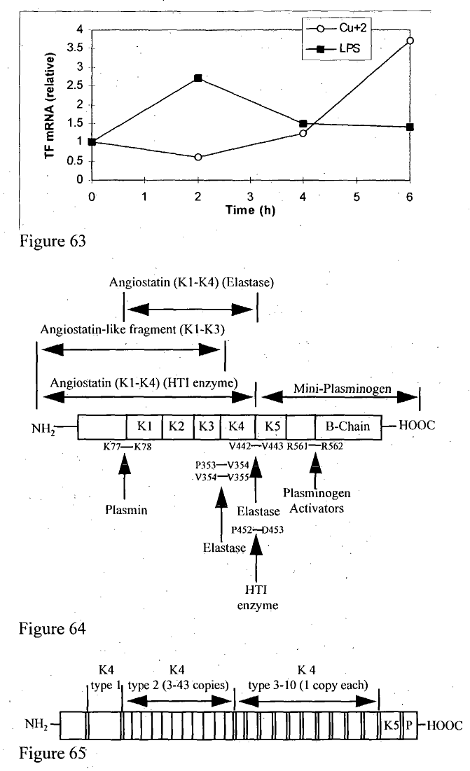

Oxidized stress increases TF transcription in monocytes and macrophages. Exposure of human THP-1 cells for 10 hours to concentrations up to 20 μmol/L Cu⁺² had no effect on procoagulant activity. However, in the presence of 1 μmol/L 8-hydroxyquinoline, Cu⁺² produced a dose dependent expression of procoagulant activity (Crutchley 1995³⁷⁵, table 1). The effect of Cu⁺² was replicated with the copper transporting protein ceruloplasmin. Cu⁺² is known to produce lipid peroxidation and free radical generation. Therefore, the study tested the possibility that the procoagulant activity results from oxidative stress. Several lipophilic antioxidants, including probucol (20 μmol/L), vitamin E (50 μmol/L), BHT (50 μmol/L), and a 21-aminosteroid antioxidant U74389G (20 μmol/L), inhibited the Cu⁺² induced procoagulant activity (Crutchley 1995, ibid, FIG. 4). The increased procoagulant activity was due to TF. Cu⁺² induced intracellular oxidative stress, which increased TF transcription. The kinetics of the induction of Cu⁺² was compared to LPS. Exposure to LPS or Cu⁺² resulted in TF mRNA increase. Relative to basal levels, LPS increased mRNA 2.5-fold after 2 hours of exposure declining to basal levels by 6 hours. In contrast, at 2 hours, Cu+2 decreased mRNA levels to 50% followed by a 3.5-fold increase at 6 hours (see FIG. 63). The Cu+2 and LPS induced TF expression also differed in the response to antioxidants. While all four antioxidant inhibited Cu+2 induced TF expression, only vitamin E inhibited the LPS induced expression.

Note: The LPS effect on TF transcription is mostly mediated through the NF-κB site. Crutchley 1995 (ibid) results indicate that oxidative stress increased TF transcription through a different DNA box. The conclusion is also supported by the negative effect of oxLDL on NF-κB binding to its site demonstrated in human T-lymphocytes (Caspar-Bauguil 1999³⁷⁶), Raw 264.7, a mouse macrophage cell line (Matsumura 1999³⁷⁷), peritoneal macrophages (Hamilton 1998³⁷⁸), macrophages (Schackelford 1995³⁷⁹), human monocyte derived macrophage (Ohlsson 1996³⁸⁰), and vascular smooth muscle cells (Ares 1995³⁸¹). The results in these studies are consistent with decreased binding of GABP to the N-box in the (−363 to −343) region of the TF gene during oxidative stress (see Appendix on the TF gene, p 267 and chapter on signaling and allocation, p 330).

Another study (Yan 1994³⁸²) tested the effect of oxLDL on TF transcription. Binding of advanced glycation end products (AGE), with their receptor (RAGE), results in intracellular oxidative stress indicated by decreased glutathione (GSH). Monocytes were incubated with AGE-albumin (AGE-alb) for 24 hours. The results showed an increase in TF mRNA (Khechai 1997³⁸³, FIG. 1B). Presence of the translational inhibitor cycloheximide completely suppressed the AGE-alb induced TF mRNA accumulation (Khechai 1997, ibid, FIG. 1B). The antioxidant N-Acetylcysteine (NAC) increases the levels GSH. NAC is easily transported into the cell. Incubation of cells with AGE-alb in the presence of 30 mmol/L NAC resulted in a concentration dependent inhibition of TF activity (Khechai 1997, ibid, FIG. 2A) and TF antigen expression. Moreover, TF mRNA was almost completely suppressed (Khechai 1997, ibid, FIG. 2C). Based on these results, Khechai, et al., (1997, ibid) concluded that oxidative stress is responsible for TF gene expression.

Crutchley 1995 (ibid) showed that although decreased oxidative stress decreases TF mRNA, the LPS induced increase in TF mRNA is insensitive to certain antioxidants. Brisseau 1995³⁸⁴ showed a similar insensitivity of the LPS induced increase in TF mRNA to the antioxidant NAC. Since Khechai 1997 (ibid) reported that NAC increases TF mRNA, the combined results in Brisseau 1995 (ibid) and Khechai 1997 (ibid) are also consistent with decreased GABP binding to the N-box in the (−363 to −343) region resulting from oxidative stress.

See also Ichikawa 1998³⁸⁵ that reported similar results in human macrophage-like U937 cells treated with the oxidant AGE and the antioxidants catalase and probucol.

Conclusion: Oxidized LDL induces oxidative stress in monocytes/macrophages. Oxidative stress increases TF transcription. Therefore, oxLDL increases TF transcription in monocytes/macrophages.

(c) Oxidized LDL and TF Transcription

Some studies tested the effect of native LDL, mmLDL, acetylated LDL (acLDL), and oxLDL on TF transcription and activity, directly.

(i) Monocytes and Macrophages

Lewis 1995 (ibid) measured TF activity in monocytes and monocyte-derived macrophages following treatment with endotoxin or minimally oxidized LDL (oxLDL). The results showed 115- and 58-fold increase in TF activity (Lewis 1995, ibid, table 1). The active peaked 4 to 6 hours after treatment and decreased over the subsequent 18 hours (Lewis, 1995, ibid, FIG. 1). Untreated cells showed little or no procoagulant activity. Lesnik 1992³⁸⁶) showed an increase in TF activity following incubation of monocytes, or monocyte-derived macrophages with acLDL. Ohsawa 2000³⁸⁷ showed an increase in TF mRNA and activity on the surface of monoblastic leukemia cells U937.

(ii) Smooth Muscle Cells (SMC)

Cui 1999³⁸⁸ showed that quiescent rat SMC contain low levels of TF mRNA. Treatment of SMC with LDL or oxLDL significantly increased TF mRNA (Cui 1999, ibid, FIG. 1). Densitometric analysis showed that oxLDL increases TF mRNA 38% more than LDL. Accumulation of TF mRNA induced by LDL or oxLDL was transient. Maximum levels of TF mRNA were observed 1.5-2 hours following LDL or oxLDL stimulation (Cui 1999, ibid, FIG. 2), declining significantly over the following 5 hours. TF mRNA response to stimulation in human aortic SMC was similar. Nuclear run-on assays, and mRNA stability experiments, indicated that the increase in TF mRNA resulted mainly from increased transcription. Penn 2000³⁸⁹ and Penn 1999³⁹⁰ reported similar effects of oxLDL and native LDL on TF mRNA in smooth muscle cells.

(iii) Endothelial Cells (EC)

Fei 1993³⁹¹ exposed human endothelial cells to minimally oxidized LDL (oxLDL), or endotoxin, for varying times. Northern blot analysis of total RNA showed an increase in TF mRNA at 1 hour, peak at 2 to 3 hours, and decline to basal levels at 6 to 8 hours after treatment. The half-life of TF mRNA, in oxLDL and endotoxin exposed endothelial cells, was approximately 45 and 40 minutes, respectively. The rate of TF mRNA degradation was similar at 1 and 4 hours post treatment. Nuclear runoff assays showed a significant increase in TF transcription rate following exposure of the cells to oxLDL or LPS.

(d) Summary

An increase in concentration of oxLDL increases the concentration of TF mRNA, symbolically,
↑[oxLDL]→↑[TF_{mRNA}]

Sequence of quantative events 8: Predicted effect of oxLDL on TF mRNA.

(ii) Prediction: Regression Diet and Plaque Stability

Define a regression diet as a decrease in fat intake following an extended period of a cholesterol-rich diet. What is the predicted effect of a regression diet on atherosclerosis? Consider the following sequence of quantitative events.

1. Macrophages (MΦ)
↓Fat intale→↓[oxLDL]→↓[TF _{mRNA}]→↓TF_{MΦ} adhesion curve→↓Skewness of V_{B, MΦ} curve→↓TotalD_{BH, MΦ}→↓(TotalD_{F, MΦ}−TotalD_{B, M+101})→↓[MΦ trapped in intima] and ↓[LDL in intima]
Sequence of quantative events 9: Predicted effect of fat intake on number of macrophages trapped in intima and concentration of LDL in the intima.

A decrease in fat intake decreases the concentration of oxLDL in the intima, decreases TF transcription in intimal macrophages, shifts-down the adhesion curve, decreases skewness of the backward velocity curve, and decreases the number of macrophages trapped in the intima.

2. Smooth Muscle Cells (SMC)

Assume a small effect of the regression diet on TF transcription, then,
↓Fat intake→↓[oxLDL]→↓[TF_{mRNA}]→↓TF_{SMC} adhesion curve→↓Skewness of V_{SMC} curve→↑TotalD_{SMC}→↑[SMC in intima]
Sequence of quantative events 10: Predicted effect of fat intake on number of SMC in intima.

A decrease in fat intake decreases the concentration of oxLDL in the media, decreases TF transcription in media smooth muscle cells, shifts-down the SMC adhesion curve, decreases skewness of the velocity curve directed toward the intima, and increases the number of SMC in the intima. Note that the decrease in the number of macrophages and the increase in the number of SMC offset each other with respect to the lesion area. Therefore, a regression diet can increase, decrease, or cause no change in lesion area. However, if the regression diet changes lesion area, the change should be small. In terms of stability, a regression diet increases plaque stability.

(iii) Observations

A study (Verhamme 2002³⁹²) fed miniature pigs chow (control group), a cholesterol-rich diet for 37 weeks (hypercholesterolemic group), or a cholesterol-rich diet for 40 weeks followed by chow for 26 weeks (cholesterol withdrawal group). The cholesterol withdrawal group showed lower plasma LDL and ox-LDL levels compared to the hypercholesterolemic group. The levels were similar to the ones observed in the control group. Atherosclerotic lesion area was 1.18±0.45, 0.88±0.70, and 0.15±0.11 mm² in the cholesterol withdrawal group, hypercholesterolemic group, and controls, respectively (non significant between cholesterol withdrawal and hypercholesterolemic groups). Lesions in the hypercholesterolemic group showed a smooth muscle cell-rich cap area, a macrophage-rich shoulder area, and a cellular-free core. Lesions in the cholesterol withdrawal group showed equal distribution of smooth muscle cells, with no macrophages or lipids. The following table summarizes the relative size of lesion area positive for macrophages, SMC, lipid; and oxLDL in the cholesterol withdrawal and hypercholesterolemic groups.

As predicted, the decrease in dietary fat decreased the number of macrophages and increased the number of smooth muscle cells in the lesion. The decrease in dietary fat also decreased the lipid content in the lesions. In addition, as predicted, the lesion area showed a small, non-significant, increase in total lesion area.

TABLE 11
Observed relative size of lesion area positive for macrophages,
SMC, lipid, and oxLDL in cholesterol withdrawal and
hypercholesterolemic group of miniature pigs.

| | Mφ | SMC** | Lipid | oxLDL |
|---|---|---|---|---|
| Cholesterol | 4.8 ± 1.7% | 29.3 ± 7.7% | 4.0 ± | 2.2 ± 16% |
| withdrawal group* | | | 2.6% | |
| Hypercholesterolemic | 20 ± 15% | 19.2 ± 3.8% | 23 ± | 12 ± 13% |
| group | | | 17% | |
| Direction | ↓ | ↑ | ↓ | ↓ |

*P < 0.05 for all differences.
**Stained for α-actin.

The study (Verhamme 2002, ibid) also measured in vitro migration of SMC isolated from coronary arteries of the miniature pigs. The study injured the cells by scraping, added 10% serum from the pigs after the cholesterol withdrawal, 10% serum from the hypercholesterolemic pigs, or 10% serum from the control pigs. After 48 hours of incubation, the study measured migration distance from the injury line and the number of cells migrated across the injury line. The results showed increased migration distance of cells treated with cholesterol withdrawal serum compared to cells treated with the hypercholesterolemic serum (data not shown in paper). The result also showed an increase in the number of cells migrating across the injury following treatment with cholesterol withdrawal serum compared to hypercholesterolemic serum. The number of cells across the injury line following treatment with cholesterol withdrawal serum was similar to the number of cells across the line following treatment with control serum.

According to the prediction above, symbolically,
↓Fat intake→↓[oxLDL]→↓[TF_{mRNA}]TF_{SMC} adhesion curves→↓Skewness of V_{SMC} curve→↑TotalD_{SMC}
Sequence of quantative events 11: Predicted effect of fat intake on TotalD_{SMC}.

A decrease in fat intake increases the total distance traveled by smooth muscle cells toward the intima (see underline). Verhamme 2002 (ibid) specially confirmed the prediction. It is interesting that the authors decided not to show data on this important observation.

Notes:

1. Other quantitative events can add to the skewness-derived effects. For instance, a decrease in recruitment of monocytes can add to the skewness-derived decrease in the number of macrophages in cholesterol withdrawal lesions. Increased SMC proliferation, or decreased apoptosis can add to the skewness-derived increase in SMC. However, with respect to cell proliferation, the study (Verhamme 2002, ibid) showed a decrease in SMC proliferation in cholesterol withdrawal lesions, inconsistent with the hypothesized added effect of SMC proliferation. In regard to cell apoptosis, the study showed a decrease in SMC apoptosis, consistent with the hypothesized added effect of SMC apoptosis. On the issue of “other quantitative events,” see also the general discussion in the introduction chapter.

2. A study (Okura 2000³⁹³) stained atherosclerosis plaque from patients undergoing carotid endarterectomy, aortic valve replacement, and femoral arterial surgery, for oxLDL. Early lesions showed oxLDL staining in the intima, and in the media just beneath the internal elastin lamina. Some of the medial oxLDL staining was localized in VSMC-derived foam cells. The oxLDL in the medial VSMC stimulate TF expression and induce migration towards the intima.

3. Other animal studies showed a decrease in the number of foam cells and regression of fatty streaks following several months of a lipid-decreased diet (Trach 1996³⁹⁴, Pataki 1992³⁹⁵, Wissler 1990³⁹⁶, Dudrick 1987³⁹⁷, Tucker 1971³⁹⁸).

4. A study (Skalen 2002³⁹⁹) reported that mice expressing proteoglycan-binding-defective LDL showed significantly less atherosclerosis compared to control mice expressing wild-type LDL. The decrease retention of apoB-containing lipoproteins decreased the rate of lesion formation. On the relation between retention of LDL in the intimal matrix and atherosclerosis, see also recent reviews: Proctor 2002⁴⁰⁰ and Williams 1998⁴⁰¹.

5. Low shear stress in the edges of blood vessel bifurcations increases LDL pollution in these areas (Malek 1999⁴⁰²). As expected, these areas show a higher propensity to develop atherosclerotic lesions.

(c) Plasminogen and Lipoprotein(a)

(i) Conceptual Background

(a) Plasminogen and Fragments

Plasminogen is a single chain glycoprotein zymogen, synthesized in the liver and circulated in the plasma at an average concentration of 2.4 mM. Plasminogen contains 790 amino acids, 24 disulfide bridges, no free sulfhydryls, one high and four low affinity lysine-binding sites, and five kringle (K) regions named after the pretzel-shaped Danish cake (see FIG. 64).

Hydrolysis of the Lys76-Lys77 peptide bond by plasmin converts the native Glu-plasminogen to Lys-77-plasminogen. Hydrolysis of the Val441-Val442 peptide bond elastase catalyzes a fragment called mini-plasminogen. Conversion of plasminogen to plasmin results from hydrolysis of the Arg560-Val561 peptide bond, yielding two chains, which remain covalently associated by a disulfide bond. Angiostatin (kringle 1-4 with or without the NH₂ terminal), and angiostatin-like fragment (kringle 1-3), are other proteolytic fragments of glu-plasminogen (see FIG. 64).

(b) Lipoprotein(a) and Apolipoprotein(a)

Lipoprotein(a) (Lp(a)) consists of the apolipoprotein(a) (apo(a)) covalently linked to the apolipoprotein B-100 (apo B). Apo(a) contains ten sequences that closely resemble the plasminogen kringle 4 (K4 type 1 to 10, or K4.1-K4.10), a kringle 5-like (K5) domain, and a protease (P) sequence. Apo(a) includes one copy of each K4 type 1, 3-10, and 3 to 43 copies of K4 type 2 (consider FIG. 65). The variable number of K4.2 sequences produces 40 distinct isoforms with molecular weight ranging from 400 to 700 kD. According to the nomenclature in Utermann 1989⁴⁰³ isoforms are classified as B. F, S1, S2, S3, and S4, where B represents small isoforms with ten or less K4.2 repeats, and S4 represents large isoforms with over 35 K4.2 repeats.

Lp(a) is synthesized in the liver and circulates in the plasma in concentrations that range between less the 1 and over 1000 mg/L.

(c) Binding and Competition

(i) TF•Plasminogen

The extracellular domain of tissue factor (TF) (amino acids 1-219) binds Glu-plasminogen with high affinity. Specifically, TF bound a plasminogen fragment that included kringle 1-3 but not an isolated kringle 4 or mini-plasminogen (Fan 1998⁴⁰⁴, ibid, FIG. 3B). The TF site that binds plasminogen seems to be different from the site that binds factors VII and VIIa.

(ii) Plasminogen•Fibronectin

A plasminogen fragment that contained kringle 1-3 or kringle 4 binds the extracellular matrix protein fibronectin (Moser 1993⁴⁰⁵, FIG. 4C and FIG. 5D, respectively). A fragment that contained kringle 1-3 or kringle 4, and the mini-plasminogen fragment also bind the extracellular matrix protein laminin (Moser 1993, ibid, FIG. 4E, 5C, 4E, respectively). Salonen 1985⁴⁰⁶ and Bendixen 1993⁴⁰⁷d reported similar binding of Glu-plasminogen to fibronectin.

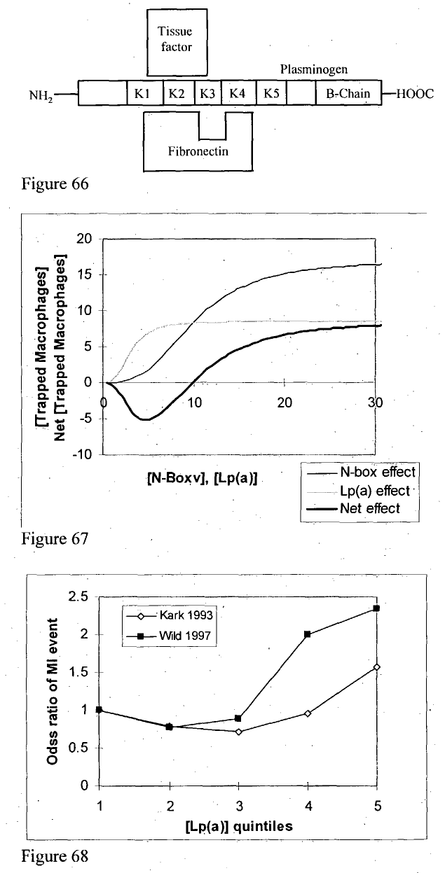

The relation between TF, plasminogen, and fibronectin is summarized in FIG. 66.

(iii) Lp(a)•Fibronectin

Lp(a) binds fibronectin (Xia 2000⁴⁰⁸), through the apo(a) kringle 4 type 2 (Kochi 1997⁴⁰⁹), the kringle with the variable number of repeats. See also Salonen 1989⁴¹⁰, and Ehnholm 1990⁴¹¹.

(iv) Lp(a) Competes with Plasminogen

Plasminogen weakly competed with apo(a) for binding to fibronectin. However, apo(a) completely abolished plasminogen binding to fibronectin (van der Hoek 1994⁴¹²). Another study (Pekelharing 1996⁴¹³) showed lysine-dependent binding of plasminogen to ECM produced by HUVECs. The study also showed that Lp(a) inhibits the plasminogen binding to the ECM in a concentration-dependent manner.

(d) Conclusion

TF propels backward motility by forming the TF·Plasminogen·Fibronectin complex (see figure above and section on TF propelled backward motility). Lp(a) competes with plasminogen for fibronectin. Therefore, an increase in Lp(a) concentration near fibronectin decreases binding of TF to fibronectin. In terms of the skewed-bell model, the increase in Lp(a) concentration shifts-down the TF adhesion curve, and decreases the skewness of the backward velocity curve. Consider the following sequence of quantitative events.
↑[Lp(a)]→↑[Lp(a)·fibronectin]→↓[Plasminogen·fibronectin]→↓[TF·plasminogen·fibronectin]→↓TF adhesion curve→↓Skewness of V_{B} curve→↑TotalD_{B}→↓(TotalD_{F}−TotalD_{B})→↓[Trapped trucking cells]
Sequence of quantative events 12: Predicted effect of lipoprotein(a) on number of trapped trucking cells.

An increase in concentration of Lp(a) decreases the number of trapped trucking cells. Lp(a) is not a cause, or risk factor for atherosclerosis, Lp(a) is an element of the trucking system that protects against the disease.

Since Lp(a) decreases the number of trapped trucking cells in the intima, a positive feedback signal should exist that modifies the concentration of Lp(a) at a certain site depending on the number of trapped cells at that site. An increase in the number of trapped cells at a certain site should increase the concentration of Lp(a) at that site. A decrease in the number of trapped cells should decrease Lp(a) concentration. Symbolically,
. . . →↑[Trapped trucking cells]_{site A}→↑[Lp(a)·fibronectin]_{site A}→ . . . →↓[Trapped trucking cells]_{site A}
Sequence of quantative events 13: Trapped trucking cells to lipoprotein(a) signal.

The symbol “ . . . →” indicates that the increase in the number of trapped trucking cells results from some unspecified preceding disruption. Subscript “site A” denotes the specific site of trucking cell accumulation and Lp(a) fibronectin complex formation. The symbol “→ . . . →” represents the above sequence of quantitative events.

Notes:

1. On Lysine

Plasminogen binds the ECM through its lysine-binding site (Pekelharing 1996, ibid). Hoek 1994 (ibid) also showed that ε-ACA, a lysine analogue, inhibited binding of plasminogen to fibronectin. However, ε-ACA was not effective against Lp(a) binding to fibronectin. These observations suggest that lysine, and lysine analogues, should be effective treatments against atherosclerosis. Note that Linus Pauling recommended using lysine as treatment against atherosclerosis, and today there is an entire industry selling lysine as a food supplement. However, Pauling based his recommendation on the erroneous assumption that Lp(a) is a injurious agent.

2. On LDL

Pekelharing 1996 (ibid) showed that LDL inhibits plasminogen binding to the ECM in a concentration-dependent manner. The LDL inhibition of the plasminogen binding can be regarded as a defensive reaction of the system. Such inhibition increases migration distance.

(ii) Predictions and Observations

(a) Net Effect

(i) Prediction

Consider an infection of monocytes with a GABP virus. The increase in number of N-boxes in the trucking cells increases the number of macrophages (Mφ) trapped in the intima (see below). Symbolically,

$$\begin{matrix}[ Trapped M ϕ ] = & f ( [ N - {box}_{v} ] ) \\ & ( + )\end{matrix} \qquad Function 26$$

The following symbolic function summarizes the inverse relation between Lp(a) in the intima and the number of trapped macrophages.

$$\begin{matrix}[ Trapped M ϕ ] = & f ( [ Lp ( a ) ] ) \\ & ( - )\end{matrix} \qquad Function 27$$

Assume that Function 26 and Function 27 are S-shaped functions. Consider the following numeric illustration.

A. Assume the following S-shaped functions represent the relations according to (VI-10) and (VI-11) over a relevant range of [N-boxᵥ] and [Lp(a)] values.

$$\begin{matrix}[ Trapped M ϕ ] = \frac{{17 [ N - {box}_{v} ]}^{3}}{{3}^{3} + {[ N - {box}_{v} ]}^{3}} & Function 28 \\ [ Trapped M ϕ ] = \frac{{8.5 [ Lp ( a ) ]}^{3}}{{10}^{3} + {[ Lp ( a ) ]}^{3}} & Function 29\end{matrix}$$

B. Define net [Trapped Mφ] as the net effect of [N-boxᵥ] and [Lp(a)] on the number of trapped macrophages, that is,
Net[Trapped ^{M}φ]=[Trapped Mφ]([N-boxᵥ])−[Trapped Mφ]([Lp(a)] Function 30

C. The graphs in FIG. 67 illustrate the values of the three functions calculated over the [0,30] range. The graphs are drawn to scale. The net effect curve is U-shaped, that is, the net number of trapped macrophages first decreases, and then increases with the increase in [Lp(a)].

Note: Other parameters for the S-shaped functions above can produce net curves with other shapes, such as continuously increasing S-shapes, or first small peak and than a U-shaped segment (see more about the choice of parameters below).

An increase in the number of trapped macrophages increases the rate of lesion formation (see above). It is well known that an increase in the rate of lesion formation increases the probability of a myocardial infarction (MI) event, or other clinical events associated with cardiovascular disease (CVD). Therefore, the function that represents the relation between Lp(a) concentration and the probability of a MI event also should show a U-shape. Consider the following observations.

(ii) Observations

A population-based case-control study (Kark 1993⁴¹⁴) recorded the Lp(a) plasma concentration in patients suffering from acute MI. The patients consisted of 238 men and 47 women, ages 25 to 64, hospitalized for a first acute MI in the 4 hospitals of Jerusalem. The control subjects comprised 318 men and 159 women sampled from the national population registry free of CHD. Another nested case-control study (Wild 1997⁴¹⁵) recorded the plasma Lp(a) level in participants of the Stanford Five-City Project, a long-term CVD prevention trial. One hundred and thirty four participants, 90 male and 44 female, with a possible or definite MI event, or coronary death, were matched with controls for age, sex, ethnicity, residence in a treatment or control city, and time of survey. Using the observed Lp(a) plasma levels, the studies calculated the odds ratio of being a case in men by quintile of Lp(a) level. The quintile cutoff points in Wild 1997 (ibid) were 6.3, 20.7, 37.5 and 112.5 nmol/L. FIG. 68 presents the results. As predicted, the curve representing the odds ratio of being a case, or the probabilities of an MI event, is U-shaped.

Note that the proposed net effect assumes, for the low to medium range of Lp(a) concentrations, that the negative effect of Lp(a) on the probability of an MI event is larger than the positive effect of the number of viral N-boxes (see the choice of parameters above). Otherwise, the predicted net effect curve will show no dip in probabilities, in contrast to the reported observations. The range where the odds ratio decline is important since it includes the only concentrations where the protective effect of Lp(a) is not masked, or overpowered, by the injurious effect of the viral N-boxes.

(b) Longevity

(i) Prediction

According to the net effect curve, the number of trapped trucking cells is smallest at a medium level of Lp(a) concentration. According to Kark 1993 (ibid) and Wild 1997 (ibid), the dip is between the second and the third quintile. Two other studies also reported a dip at medium Lp(a) levels. Rhoads 1986⁴¹⁶ reports an odds ratio of 0.75 for a MI event at the third quartile defined by the 10.8-20.1 mg/dl range of Lp(a) concentrations, and Kronenberg 1999A⁴¹⁷ reports an odds ratio of 0.5 for showing advanced atherogenesis at the range of 24-32 mg/dl of Lp(a) concentrations. Consider the following sequence of quantitative events.
Medium level of Lp(a)→Minimum net [Trapped Mφ]→Minimum [lesions]→Maximum contribution to life expectancy
Sequence of quantitative events 14: Predicted effect of lipoprotein(a) on life expectancy.

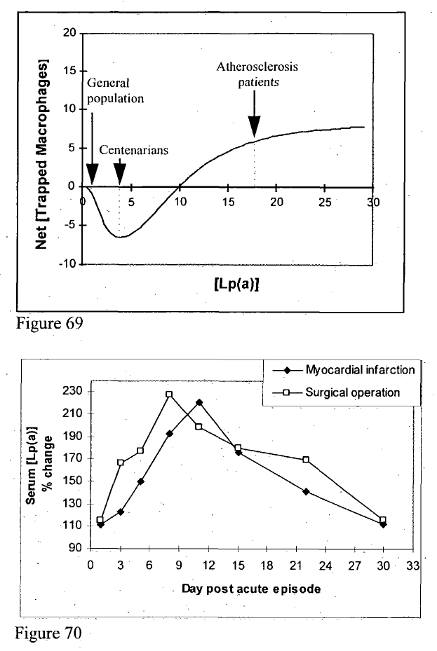

A medium level of Lp(a) should be associated with longevity. The general population should show low Lp(a) levels, centenarians should show medium levels, and atherosclerosis patients should show high levels of Lp(a). The prediction is summarized in FIG. 69.

(ii) Observations

A study (Thillet 1998⁴¹⁸) recorded the Lp(a) levels in a population of 109 French centenarians and 227 controls. The mean age of centenarians and controls was 101.5±2.4, and 39.4±7.2 years, respectively. Plasma levels of total cholesterol and triglyceride were within the normal range in both groups. Average plasma Lp(a) levels in centenarians and controls was 33 and 21 mg/dl, respectively (p<0.005). Moreover, the distribution of Lp(a) concentration showed 28% of the centenarians at concentrations of 10-20 mg/dl and 30% at concentration of 10-20 mg/dl, while the distribution showed 49% of controls at concentrations of 0-10 mg/dl and 19% at concentrations of 10-20 mg/dl (Thillet 1998, ibid, FIG. 1). Based on these observations, Thillet, et al., (1998, ibid) concluded: “By studying a unique and, large sample of centenarians, we have shown that circulating Lp(a) are significantly increased in this group as compared to younger, normolipidemic, control subjects.” As predicted, centenarians showed a higher average Lp(a) level relative to the general population.

Note that another study (Baggio 1998⁴¹⁹) also reports higher average plasma Lp(a) in 75 healthy centenarians compared to 114 randomly selected subjects with average age of 35.8 years (22.4 vs. 19.3 mg/dl, respectively). However, the difference was not significant statistically.

(c) Inverse Relation

(i) Prediction

Lp(a) binds fibronectin through the apo(a) kringle 4 type 2 (see above). Assume that one apo(a) molecule can bind many fibronectin molecules, and that there exists a direct relation between the number of apo(a) kringle 4 type 2 repeats and the number of bound fibronectin molecules. Also assume that the number of trapped trucking cells regulates the plasma level of Lp(a) through synthesis or degradation (see more on this assumption in the section entitled “Co-occurrence (acute-phase reactant)” below). Consider the following sequence of quantitative events.
↑[Apo(a) KIV-2]→↑[Lp(a)·fibronectin]→ . . . →↓[Trapped trucking cells]→↓[Lp(a)]plasma
Sequence of quantative events 15: Predicted effect of apo(a) kringle 4 type 2 concentration on plasma lipoprotein(a) concentration.

As before, the symbol “→ . . . →” represents the above sequence of quantitative events. An increase in the number of apo(a) kringle 4 type 2 repeats should decrease plasma Lp(a). The sequence of quantitative events predicts an inverse relation between the number of apo(a) KIV-2 and plasma Lp(a). Since the number of KIV-2 repeats determines the size of the Lp(a) molecule, the sequence of quantitative events also predicts an inverse relation between size and plasma Lp(a).

(ii) Observations

Many studies reported an observed inverse relation between size of Lp(a), or the number of KIV-2 repeats, and plasma Lp(a), see for instance, DePrince 2001⁴²⁰, Chiu 2000⁴²¹, Valenti 1999⁴²², Gaw 1998⁴²³, Valenti 1997⁴²⁴. See also two recent reviews, de la Pena-Diaz 2000⁴²⁵ and Pati 2000⁴²⁶.

(d) Co-Localization with Extracellular Matrix

(i) Prediction

The biological function of apo(a) is competition with plasminogen for binding with fibronectin in the intima. Therefore, apo(a) should be found mostly extracellularly, specifically, bound to the extracellular matrix.

(ii) Observations

Many studies reported locating apo(a) extracellularly in the intima, see for instance, Beisiegel 1990⁴²⁷, Rath 1989⁴²⁸. Studies with transgenic animals specifically reported observing apo(a) bound to the extracellular matrix, see for instance, Ichikawa 2002⁴²⁹ and Fan 2001⁴³⁰.

(e) Co-Localization with Plaque

(i) Prediction

Consider the positive feedback signal that links the number of trapped trucking cells at a certain site with the Lp(a) concentration at that site (see above).
↑[Trapped trucking cells]_{site A}→↑[Lp(a)]_{site A}
Sequence of quantative events 16: Predicted effect of trapped trucking cells at a certain site on lipoprotein(a) at that site.

Lp(a) should be found at sites of macrophage accumulation. Since a high number of trapped cells co-localize with plaques, Lp(a) should also co-localize with plaque.

(ii) Observations

A study (Dangas 1998⁴³¹) examined coronary atheroma removed from 72 patients with stable or unstable angina. Specimens were stained with antibodies specific for Lp(a) and macrophages (KP-1). The study used morphometric analysis to quantify the plaque areas occupied by each antigen, and their co-localization. The results showed localized Lp(a) staining, in which 90% of the macrophage areas co-localized with Lp(a) positive areas. Based on this observation Dangas, et al., (1998, ibid) concluded: “Lipoprotein(a) . . . has significant co-localization with plaque macrophages.”

In general, many studies showed co-localization of Lp(a) with plaque, see for instance, Reblin 1995⁴³², Hoff 1993⁴³³, Kusumi 1993⁴³⁴, Pepin 1991⁴³⁵, and Rath 1989 (ibid). Studies with transgenic animal reported similar co-localization, see for instance, Ichikawa 2002 (ibid), Boonmark 1997⁴³⁶, Lawn 1992⁴³⁷.

(f) Angiogenesis

(i) Prediction

Angiogenesis is the process where pre-existing capillaries form new blood vessels. A regular level of angiogenesis can be found in normal tissue growth, such as in wound healing, and the menstrual cycle. However, excessive angiogenesis was observed in several diseases, such as cancer, atherosclerosis, chronic inflammation (rheumatoid arthritis, Crohn's disease), diabetes (diabetic retinopathy), psoriasis, endometriosis, and adiposity (Griffioen 2000⁴³⁸, Reijerkerk 2000⁴³⁹).

Angiogenesis includes a phase of endothelial cell migration. Angiostatin is a fragment of plasminogen that includes kringles 1-3, the binding sites for tissue factor (TF) and for fibronectin (fibronectin also binds kringle 4). Therefore, an angiostatin K1-3 should have the same effect as plasminogen on endothelial cell (EC) motility. Consider the following sequence of quantitative events.

↑[Angiostatin (K1-3)]→↑[TF·Angiostatin (K1-3)·fibronectin]→↑TF adhesion curve→↑Skewness of V_{EC} curve→↓TotalD_{EC}→↓[Angiogenesis]
Sequence of quantative events 17: Predicted effect of angiostatin (K1-3) on angiogenesis.

An increase in concentration of an angiostatin fragment that includes kringles 1-3 shift-up the TF adhesion curve, increases the skewness of the velocity curve of the endothelial cell, decreases the total distance traveled by the cell, and decreases the rate of angiogenesis.

Lp(a) inhibits binding of plasminogen to fibronectin. Therefore, Lp(a) should show an angiogenic effect.
↑[Lp(a)]→↑[Lp(a)·fibronectin]→↓[Plasminogen·fibronectin]→↓[TF·plasminogen·fibronectin]→↓TF adhesion curve→↓Skewness of V_{EC} curves→↑TotalD_{EC}→↓[Angiogenesis]
Sequence of quantative events 18: Predicted effect of lipoprotein(a) on angiogenesis.

An increase in Lp(a) should increase the rate of angiogenesis. Since the concentrations of angiostatin and Lp(a) are self regulated, these predictions can be further extended to include predictions such as increased angiostatin and decreased Lp(a) in cancer, decreased angiostatin and increase in Lp(a) in injury, etc.

(ii) Observations

As expected, several studies reported an inverse relation between angiostatin and angiogenesis (see for instance, O'Reilly 1994⁴⁴⁰). In addition, a study reports elevated levels of urine angiostatin and plasminogen/plasmin in cancer patients relative to controls (Cao Y 2000⁴⁴¹). Also, as expected, a study showed increased angiogenesis in gelatin sponges loaded with Lp(a) implanted in vivo onto a chick embryo chorioallantoic membrane (CAM) (Ribatti 1998⁴⁴², table 1). The magnitude of the effect was similar to that obtained with FGF-2, a well-known angiogenic molecule (Ribatti 1998, ibid, table 1). Application of anti-Lp(a) antibodies on the CAM significantly inhibited the observed angiogenesis (Ribatti 1998, ibid, table 1), which indicates that the effect was specific (Ribatti 1998, ibid, table 1).

Note: Since angiogenesis also includes a phase of cell proliferation, direct observations of the effect of angiostatin and Lp(a) on cell migration in vivo will increase the validity in the proposed relation.

(g) Defensin

(i) Conceptual Background

α-defensins are small (29 to 35 amino acid) peptides released by activated neutrophils. Defensins incorporate into the cell membrane of eukaryotic organisms within phagolysosomes, disrupting ion fluxes, and inducing cell lysis. Defensin (5 to 10 μmol/L) increased binding of ¹²⁵I-Lp(a) and ¹²⁵I-apo(a) to fibronectin coated microtiter wells, by 30- and 20-fold, respectively (Bdeir 1999⁴⁴³, FIG. 8A, 9A). Defensin also stimulated binding of fibronectin at a concentration (50 nmol/L) where independent binding to apo(a) could not be observed. Binding of Lp(a) to fibronectin increased in a dose-dependent manner (Bdeir 1999, ibid, FIG. 8B2). Binding of defensin·Lp(a) complexes to the extracellular matrix was more than 63.3% inhibited by anti-fibronectin antibodies (Bdeir 1999, ibid). The study also showed that defensin inhibits Lp(a) endocytosis and degradation. These observations suggest that defensin stimulated binding of Lp(a) and apo(a) to fibronectin and retention on the extracellular matrix.

(ii) Prediction

Consider the following sequence of quantitative events.
↑[Defensis]_{site A}→↑[Lp(a)·fibronectin]_{site A}→↓[Plasminogen·fibronectin]_{site A}→↓[TF·plasminogen·fibronectin]→↓TF adhesion curve→↓Skewness of V_{B} curve→↑TotalD_{B}→↓(TotalD_{F}−TotalD_{B})→↓[Trapped trucking cells]→↓[Lesion]
Sequence of quantative events 19: Predicted effect of defensis at a certain site on lesion formation at that site.

An increase in defensin near the Lp(a)•fibronectin complex decreases the number of trapped trucking cells and the rate of lesion formation. Defensin is also an element of the trucking system that protects against atherosclerosis.

(iii) Observations

Direct observations of the relation between defensin and the rate of lesion formation are not available. However, to decrease the rate of lesion formation, defensin should co-localize with Lp(a). Consider the following observations.

A study (Higazi 1997⁴⁴⁴) analyzed the expression of defensin in human atherosclerotic vessels. The study observed co-localization of defensin and apo(a) in areas of vessel involved with atherosclerosis, specifically, in the intima. The study also observed close correlation between the distribution and intensity of staining for defensin and apo(a) and the severity of the disease as indicated by the stage and morphology of the plaque. In areas with normal vessel morphology and thickness, where the endothelium was intact, the study observed little or no defensin or Lp(a), although neutrophils within the lumens of the vessels stained intensely (Higazi 1997, ibid, FIG. 6). The observations are consistent with the proposed protective effect of defensin against lesion formation.

(h) Injury and Wound Healing

(i) Co-Localization

Prediction

In injury, trucking cells migrate to the site of injury, load foreign elements and cell debris, and then migrate out, carrying the accumulated particles to a target tissue, such as a lymph node (see chapter on autoimmune disease, p 272).

An increase in trucking cell traffic at the site of injury increases the number of trucking cells trapped at the site (see % trapped above). Consider the positive feedback signal that links the number of trapped trucking cells at a the site of injury with the Lp(a) concentration at that site (see above).
↑[Trapped trucking cells]_{injury site}→↑[Lp(a)]_{injury site}
Sequence of quantative events 20: Predicted effect of trapped trucking cells at a certain site on lipoprotein(a) at that site.

Lp(a) should be found at sites of trucking cell accumulation. Since a high number of trapped cells is found at sites of injury, Lp(a) should also co-localize with sites of injury, but not with control sites (this prediction is similar to the prediction presented in the section entitled “Co-localization with plaque,” see above).

An increase in Lp(a) concentration at the site of injury decreases the number of trucking cells trapped at the site, which decreases the time between assault and recovery. The increase in Lp(a) concentration at the site of injury also stimulates angiogenesis, which further decreases the time between injury and healing. Lp(a) is not only an element of the LDL trucking system, but also an element of the immune and angiogenesis systems.

Observations

A study (Yano 1997⁴⁴⁵) classified four stages of wound healing. Early in the first stage (denoted Ia), fibrin clots form over the bare surface of the wound. Later in the first stage (denoted lb), inflammatory cells infiltrate the site of the wound. In the second stage, the base of the coagulum is replaced by granulation tissue. During the second stage, granulation tissue is often covered with loose fibrous connective tissue with various thickness, designated as a “fibrous cap.” The second stage is also characterized by angiogenesis. In the third stage, epithelial sheets are spread to cover the granulation tissue. In the fourth stage, collagen fibers replace the granulation tissue, which decreases the size of the wound. Replacement of granulation tissue with new epithelium, or by organization, completes the healing process.

The study stained 50 samples from abscess, ulcers, granulation tissues, scars, polyps, and foreign body granulomas, on skin, external ear, nasal cavity, larynx, tongue, soft palate, stomach, colon, and carotid artery (Yano 1997, ibid, table 1) with anti-apo(a) antibodies. Normal tissue showed no apo(a) staining. In wounds, during Stage 1a, about one fourth of the specimens showed anti-apo(a) staining. In Stage 1b, more specimens showed positive staining (Yano 1997, ibid, table 3). During the lb stage, apo(a) was localized at the site of necrotic debris, inflammatory cell-infiltration, in small vessels, and in the extracellular space (Yano 1997, ibid, FIG. 2). In the second stage, apo(a) showed markedly enhanced staining on the fibrous cap. Apo(a) was also observed in endothelial cells and in the extracellular space around the small vessels underlying the fibrous cap (Yano 1997, ibid, FIG. 4). In the third stage, apo(a) staining became weaker with re-epithelization of the wound (Yano 1997, ibid, table 3). Tissues resurfaced with epithelium showed no apo(a) staining. Un-epithelized surfaces in the same tissue still stained for apo(a) (Yano 1997, ibid, FIG. 6). In the last stage, endothelial cells and the extracellular matrix in completely organized tissue showed no apo(a) staining, however, the vascular walls at the site infiltrated with inflammatory cells still showed apo(a) staining (Yano 1997, ibid, FIG. 7).

In injury, trucking cells are trapped near cell debris while they traverse the extracellular space. As expected, in stage 1b, apo(a) was observed in the extracellular space at the site of necrotic debris and inflammatory cell-infiltration. Apo(a) promotes migration of endothelial cells, which stimulates angiogenesis. As expected, during the second stage, when angiogenesis occurs, apo(a) was observed in endothelial cells and in the extracellular space around small vessels underlying the fibrous cap. The results in Yano 1997 are consistent with the proposed effect of Lp(a) of cell migration.

Another study (Ryan 1997⁴⁴⁶) used an angioplasty catheter to distend the iliac artery of male cynomolgus monkeys with midrange Lp(a) levels. The pressure resulted in focal breaks in the internal elastic lamina (IEL) in 80% of the vessels, and considerable IEL fragmentation with medial disruption in 20% of the vessels. The study examined Lp(a) localization in injured and control arteries using a mouse monoclonal anti-Lp(a) antibody. Control arteries showed no Lp(a) staining. All 10 injured arteries showed positive staining at the site of injury. All injured arteries showed neointimal growth; thrombus formation was observed in 40% of the vessels. Lp(a) staining was associated with the thrombus. However, staining was also observed at some distance from the thrombus in both the neointima and the media. Similar results are reported in Ryan 1998⁴⁴⁷. Based on these observations, Ryan, et al., (1997, ibid) concluded: “In the present study we showed that Lp(a) is deposited only at the site of vascular injury.” Moreover, the study suggests that “Lp(a) uptake is specific.”

Another study (Nielsen 1996⁴⁴⁸) showed a much larger accumulation of Lp(a) in balloon-injured rabbit aorta in vivo compared to normal vessels. The study compared Lp(a) and LDL accumulation at the site of injury. Concurring with Ryan 1997, the study concluded: “the data support the ideas of a specific accumulation of Lp(a) compared with LDL in injured vessels.” The results in Ryan 1997 (ibid), Ryan 1998 (ibid), and Nielsen 1996 (ibid) are consistent with the proposed effect of Lp(a) on cell migration.

(ii) Co-Occurrence (“Acute-Phase Reactant”)

Prediction

Assume that the number of trapped trucking cells regulates the plasma level of Lp(a) through synthesis or degradation (see also the section entitled “Inverse relation” above). Symbolically,
↑[Trapped trucking cells]ₜ→↑[Plasma I.p(a)]ₜ₊₁
Sequence of quantative events 21: Predicted effect of trapped trucking cells at time t on plasma level of lipoprotein(a) at time t+1.

An increase in the number of tapped trucking cells at time t increases the plasma level of Lp(a) at time t+1. A decrease in the number of trapped cells subsequently decreases the plasma level of Lp(a).

Notes:

1. Extensive injuries, such as myocardial infarctions or surgical operations, result in a large increase in the number of trapped trucking cells, and a substantial increase in plasma Lp(a). Small scale injuries might not produce a detectable effect on plasma Lp(a).

2. Apo(a) isoforms with higher numbers of apo(a)-kringle 4 type 2 repeats, or larger size, are more effective in decreasing the number of trucking cells trapped at the site of injury. Therefore, the plasma level of larger size apo(a) isoforms should increase more than the plasma level of the smaller size isoforms.

Observations

A study (Maeda 1989⁴⁴⁹) measured serum Lp(a) level over time following an acute attack of myocardial infarction, or a surgical operation, in 21 and 11 patients, respectively. The average initial Lp(a) level for the myocardial and the surgical operation patients was 18.1 and 18.8 mg/dI, respectively. FIG. 70 presents the results (Maeda 1989, ibid, FIG. 2A).

As expected, plasma Lp(a) first increased and then decreased. Based on these observations, Maeda, et al., (1989, ibid) concluded: “The role of Lp(a) is at the present a matter of speculation. One possibility is that Lp(a) reacts like an acute phase reactant and may play an important role, at least in part, in recovery from tissue damage.”

A follow-up study (Noma 1994⁴⁵⁰) analyzed the relative concentration of apo(a) isoforms in patients from a similar population with a double-band phenotype, that is, patients that express two apo(a) isoforms. The results showed that, following the episodes, plasma level of the higher-density Lp(a) particles increased more than the lower-density Lp(a) particles. The ratio of the higher- to lower-density Lp(a) particles was 0.75 at the initial time, and greater than 1.0 during peak time. Note that the higher-density Lp(a) particles preferentially contain apo(a) isoforms with a higher number of K4 type 2 kringles. Based on the observations, Noma, et al., (1994, ibid) concluded: “The present findings suggest that Lp(a) may play an important role as an acute-phase reactant, as well as other proteins, in the repair of tissue injury, especially in the process of angiogenesis.” The conclusion agrees with the proposed effect of Lp(a) on angiogenesis.

Another study (Min 1997⁴⁵¹) observed significantly (p<0.0001) higher serum Lp(a) in patients with an acute-phase response (APR) compared to controls. Moreover, the mean serum Lp(a) concentration of the most frequently occurring apo(a) phenotypes (S5, S4S5, S5S5, and S4) was substantially higher. In the discussion, Min, et al., (1997, ibid) write: “Kawade, et al. [15] reported that patients whose Lp(a) concentration reached a peak on the 5^{th} to 10^{th} day after surgery and then returned to the initial value in 1 week had a good prognosis, whereas those who did not experience the transient increases of Lp(a) had a poor prognosis. These findings could be interpreted to mean that Lp(a) played an important role in the patients' recovery from the injuries of surgery.” The cited observations and the interpretation in Min 1997 agree with the proposed effect of Lp(a) (see also Lp(a) and patient survival next).

(i) Patient Survival

(i) Prediction

An apo(a) isoform with a smaller number of kringle 4 type 2 repeats is less effective in modifying cell motility. Consider two individuals with different apo(a) isoforms. The individual with the lower number of kringle 4 type 2 repeats will show a higher level of plasma Lp(a) (see section entitled “Inverse relation” above). Assume the increase in plasma Lp(a) does not fully compensate for the decreased effectiveness of the smaller apo(a). Under such condition, the individual with the larger apo(a) should show better prognosis in disease.

(ii) Observations

A study (Kronenberg 1999B⁴⁵²) investigated the effect of apo(a) size on survival of type I diabetes mellitus patients. The study included patients with at least one small apo(a) isoform, that is, 11 to 22 kringle 4 repeats, in the low molecular weight group (LMW). Subjects with only large isoforms, that is, more than 22 kringle 4 repeats, were included in the high molecular weight group (HMW). The results showed an inverse relation between the percent of LMW phenotypes in the population of patients and the duration of the disease (p=0.001, Mantel-Haenszel test for linear association). The percent of LMW phenotypes decreased from 41.7% in patients with 1-5 years to only 18.2% in patients with 35 years duration of disease (Kronenberg 1999B, ibid, FIG. 1). The study also tested the relation in the tertiles with short (1-15 years) and long duration (>27 years). The percent of LMW phenotypes was substantially higher in patients with short compared to long duration (38.9% vs. 22.4%, p=0.009). Based on these observations, Kronenberg, et al., (1999, ibid) concluded that LMW apo(a) isoforms are associated with a disadvantage in long-term survival of type I diabetes mellitus patients. In other words, HMW apo(a) isoforms are associated with an advantage in long-term survival of type I diabetes mellitus patients.

Another study (Wahn 2001⁴⁵³) examined the long-term effect of apo(a) size on long-term graft survival in patients who received a renal transplant. The study used a grouping of patients similar to Kronenberg 1999B (ibid). The results showed that in patients 35 years or younger at time of transplantation, mean graft survival was more than 3 yr longer in recipients with HMW apo(a) phenotypes compared to LMW phenotypes (13.2 vs. 9.9 years, p=0.0156). Based on their observations, Wahn, et al., (2001, ibid) concluded: “These retrospective data indicate that young renal transplant recipients with LMW apo(a) phenotypes have a significantly shorter long-term graft survival, regardless of the number of HLA mismatches, gender, or immunosuppressive treatment.” In other words, young renal transplant recipients with HMW apo(a) phenotypes have a significantly longer long-term graft survival.

The observations in Kronenberg 1999B (ibid) and Wahn 2001 (ibid) are consistent with the proposed effect of Lp(a) on cell motility.

Note that Lp(a) should show the same effect on cell motility in autoimmune disease. As in other kinds of injury, trucking cells mobilize cell debris and foreign elements from the site of the injured organ to target sites (see chapter on autoimmune disease, p 272). As detailed above, an increase in Lp(a) concentration at the original site of injury decreases the number of trucking cells trapped at that site. In addition to atherosclerosis, many of the above predictions can also be tested in autoimmune disease, see for example, Kronenberg 1999B (ibid) in type I diabetes.

(j) Transgenic Animals and Plaque Stability

(i) Prediction

A study (Fan 2001, ibid) generated transgenic rabbits expressing the human apo(a), which was associated with rabbit apoB to form Lp(a)-like particles in the plasma. The study fed transgenic rabbits a cholesterol-rich diet. Another group of transgenic rabbits was fed a chow diet. Two more groups of non-transgenic rabbits were fed a cholesterol-rich diet and a chow diet.

Macrophages (Mφ)

What is the effect of the apo(a) transgene on the relative number of macrophages in the intima? Consider the following sequences of quantitative events.

A. Apo(a) transgenic rabbits vs. non-transgenic rabbits fed a chow diet:
↑[Cholesterol in diet]→↑[Trappedtrucking cells]ᵢₙₜᵢₘₐ→↑[Lp(a)·fibroprotein]ᵢₙₜᵢₘₐ→ . . . →↑[Lesion]
Sequence of quantative events 22: Predicted effect of a chow diet on lesion formation.

Since there is no increase in the cholesterol in the diet, there is no increase in the number of trapped trucking cells in the intima, resulting in no increase in Lp(a) in the intima, and no change in rate of lesion formation. To conclude, under a chow diet, apo(a) transgenic rabbits should show no increase in lesion formation relative to non-transgenic rabbits.

B. Apo(a) transgenic rabbits vs. non-transgenic rabbits fed a cholesterol-rich diet:

Non-Transgenic Rabbits:
↑[Cholesterol in diet]→↑[Trapped trucking cells]ᵢₙₜᵢₘₐ
Sequence of quantative events 23: Predicted effect o cholesterol-rich diet on number of trapped trucking cells in non-transgenic rabbits.
Apo(a) Transgenic Rabbits:
↑[Cholesterol in diet]→↑[Trapped trucking cells]ᵢₙₜᵢₘₐ→↑[Lp(a)·fibronectin]ᵢₙₜᵢₘₐ→ . . . →↓[Trapped trucking cells]_{site A}
Sequence of quantative events 24: Predicted effect of cholesterol-rich diet on number of trapped trucking cells in apo(a) transgenic rabbits.

Under a cholesterol-rich diet, the apo(a) transgenic rabbits should show a decreased number of trapped trucking cells relative to non-transgenic rabbits. Macrophages are trucking cells; therefore, the transgenic rabbits should show a relative decrease in the number of macrophages in the intima.

Smooth Muscle Cells (SMC)

What is the effect of the apo(a) transgene on the number of smooth muscle cells (SMC) in the intima?

Consider a SMC in the media. Under a cholesterol-rich diet, a SMC starts to migrate towards the intima. The total distance traveled by the cell can be expressed as the area under the SMC velocity curve (see chapter on cell motility, p 145). Consider the following sequence of quantitative events.

Non-Transgenic Rabbits:
↑[Cholesterol in diet]→↑[Trapped trucking cells]ᵢₙₜᵢₘₐ
Sequence of quantative events 25: Predicted effect of o cholesterol-rich diet on number of trapped trucking cells in non-transgenic rabbits.
Apo(a) Transgenic Rabbits:
↑[Cholesterol in diet]→↑[Trapped trucking cells]ᵢₙₜᵢₘₐ→↑[Lp(a)·fibroprotein]ᵢₙₜᵢₘₐ→↓[Plasminogen·fibronectin]→↓[TF_{SMC}·plasminogen·fibronectin]→↓TF_{SMC} adhesion curve→↓Skewness of V_{SNC} curve→↑TotalD_{SMC}→↑[SMC in intima]
Sequence of quantative events 26: Predicted effect o cholesterol-rich diet on number of SMC in the intima in apo(a) transgenic rabbits.

The apo(a) transgene shifts-down the SMC adhesion curve, decreases the skewness of the SMC velocity curve, increases the distance traveled by the SMC toward the intima, resulting in more SMC arriving to the intima.

To conclude, under a cholesterol-rich diet, the apo(a) transgenic rabbits should show increased number of smooth muscle cells in the intima relative to non-transgenic rabbits.

In terms of plaque stability, apo(a) transgenic mice should show plaque with higher stability.

(ii) Observations

As expected, the aorta, coronary artery, and cerebral artery in transgenic and non-transgenic rabbits on standard chow diet failed to show any atherosclerotic lesions (Fan 2001, ibid).

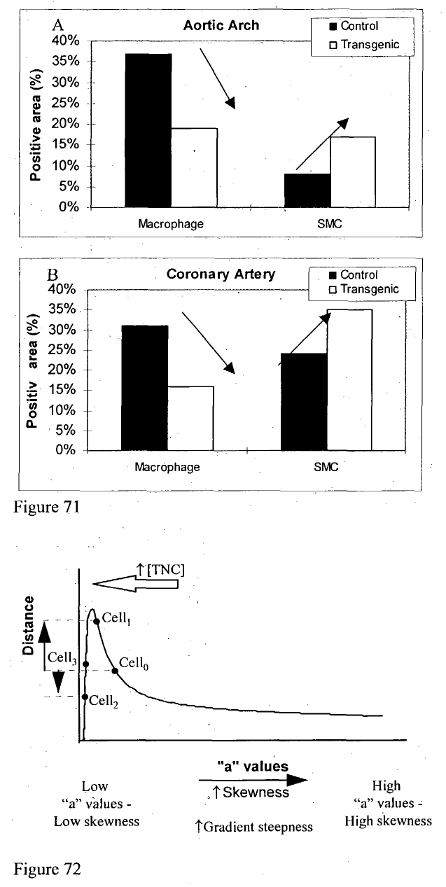

In a continuation study, Ichikawa, et al., (2002, ibid) reported that the atherosclerosis lesions in transgenic rabbits contained relatively more SMC and fewer macrophages compared to non-transgenic rabbits in both the aorta and coronary artery. FIG. 71 presents the observations.

As expected, under a cholesterol-rich diet, the apo(a) transgenic animals showed decreased number of macrophages and increased number of SMC in the intima, relative to non-transgenic animals.

Moreover, the study also reports that the SMC in the intima were activated and immature (Ichikawa 2002, ibid, FIG. 6). Also consistent with the predicted effect of the apo(a) transgene on SMC migration is that SMC typically migrate only as immature cells (Witzenbichler 1999⁴⁵⁴).

(iii) Summary

Currently, there is a strong consensus in the research community that Lp(a) promotes atherosclerosis. Some even assign to the Lp(a) atherogenic effect major significance. Consider, for example, Lippi 2000⁴⁵⁵: “We review current concepts regarding the genetic, structural and metabolic features of lipoprotein(a), a major inherited cardiovascular pathogen,” or Kostner and Kostner (2002⁴⁵⁶): “Lipoprotein(a) belongs to the class of the most atherogenic lipoproteins.” The consensus is so strong that pharmaceutical companies are currently attempting to develop drugs to decrease the level of Lp(a) in the plasma. See, for instance, the newly approved extended-release formulation of niacin, a drug that significantly decreased Lp(a) by 27% at a dosage of 2 g administered daily (Scanu 1998⁴⁵⁷). However, the same community also admits that “We are still far away from understanding . . . the physiological function of this lipoprotein” (Kostner 2002, ibid), or “Although lipoprotein(a) (Lp[a]) has been recognized as an atherothrombogenic factor, the underlying mechanisms for this pathogenicity have not been clearly defined” (Scanu 1998, ibid). Scanu finds this lack of understanding disturbing, “we cannot truly assess the cardiovascular pathogenicity of Lp(a) without a clear understanding of what goes on in the artery” (Scanu 1998, ibid). Scanu also cites observations inconsistent with the accepted atherogenic effect of Lp(a): “the notion of an inverse relation between apo(a) size, plasma Lp(a) levels, and cardiovascular risk is not compatible with the following observations: (1) studies of African Americans, in whom cardiovascular risk is not proportional to plasma Lp(a) levels; (2) uncertainties about the precise cutoff point for “pathologic” plasma Lp(a) levels, reflecting ethnic variations and lack of standardization of Lp(a) assays; (3) evidence that the atherothrombogenic potential of Lp(a) many be influenced by other risk factors, including plasma levels of LDL, high-density lipoprotein (HDL), and homocysteine” (Scanu 1998, ibid). Hobbs and White find issues with the “apparent contradictory findings that Lp(a) is an important independent risk factor (cross-sectional and retrospective studies) and a marginal risk factor (prospective studies) for coronary artery disease” (Hobbs 1999⁴⁵⁸). However, none of these reviews deviates from the consensus. They all agree on the atherogenic effect of Lp(a). The only publication I found through Pubmed that expressed nonconforming views was a letter by Goldstein. According to Goldstein 1995⁴⁵⁹: “What comes first, the chick or the egg? It is possible that elevated Lp(a) levels occur in response to tissue injury, whether it is the blood vessel wall or elsewhere. It is also possible that elevated Lp(a) levels do not primarily cause arterial injury. . . . Lp(a) is elevated after surgery and myocardial infarction and may play a role in the repair of damaged tissues. Long distance runners and weight lifters have elevated Lp(a) levels. It is known that exercise protects against atherosclerosis, and therefore, it is a paradox that athletes may have elevated Lp(a) levels.” However, even Goldstein agrees with the atherogenic effect of Lp(a): “Lp(a) might be a double edged sword.” . . . “Lp(a) may be a friend or foe depending on the situation.” (Note that Goldstein suggests that the positive role of Lp(a) in atherosclerosis is the delivery of cholesterol to areas of tissue damage).

The specifications present a model that describes the physiological function of Lp(a). In contrast to the current consensus, the physiological function suggests that Lp(a) protects against atherosclerosis.

(d) Calmodulin Antagonists

(i) Conceptual Background

Several studies reported decreased cell attachment to fibronectin, and other extracellular matrix proteins, following treatment with Calmodulin (CaM) antagonists. For instance, Mac Neil 1994A⁴⁶⁰ used six ocular melanoma cell lines established from choroidal melanoma tumors. The study showed significant inhibition of cell attachment to plates coated with fibronectin, collagen type I, III, IV, laminin, gelatin, RGD, vitronectin or poly-1-lysine, following treatment with the CaM antagonists tamoxifen and J8 (Mac Neil 1994A, ibid, FIGS. 1 and 2C, table 2). See similar results in Mac Neil 1994B⁴⁶¹. Significant inhibition was also observed following treatment with the calcium ionophore ionomycin (Mac Neil 1994A, ibid, table 2). Another study (Millon 1989⁴⁶²) showed decreased attachment of the ZR75-1 line of breast cancer cells to the extracellular matrix (Millon 1989, ibid, FIG. 1A), and to plates coated with fibronectin, collagen type I or IV (Millon 1989, ibid, table 1), following treatment with tamoxifen. Another study (Wagner 1995⁴⁶³) showed decreased attachment of retinal pigment epithelial (RPE) cells to fibronectin following treatment with the CaM antagonists tamoxifen and J8, even after cells had been allowed to adhere for 24 hours prior to exposure (Wagner 1995, ibid, FIG. 2, 6, 7). Tamoxifen and J8 also decreased attachment to collagen type I, III, IV, laminin, gelatin, RGD, vitronectin, or poly-1-lysine. Tamoxifen also decreased attachment to plastic (Wagner 1995, ibid, FIG. 8). These observations suggest that tamoxifen, most likely, also decreases attachment of trucking cells to fibronectin.

(ii) Prediction

Consider the following sequence of quantitative events.
↑[tamoxifen]→↓TF adhesion curve→↓Skewness of V_{B} curve→↑ToralD_{B}→↓(TotalD_{F}−TotalD_{B})→↓[Trapped trucking cells]ᵢₙₜᵢₘₐ→↓[Lp(A)]ᵢₙₜᵢₘₐ
Sequence of quantative events 27: Predicted effect of tamoxifen on lipoprotein(a) in intima.

Treatment with tamoxifen shifts-down the TF adhesion curve, decreases the skewness of the velocity curve, decreases the number of trapped cells in the intima, and decreases the concentration of Lp(a) in the intima.

(iii) Observations

A study (Lawn 1996, ibid) fed apo(a) transgenic mice a cholesterol-rich diet with and without 15 pig of tamoxifen. After 12 weeks, the study measured lesion formation in aortic sections. Tamoxifen decreased the number of lipid lesions by 80% and lesion area by 92% (Lawn 1996, ibid, table II). Tamoxifen also decreased the average level of apo(a) in the vessel wall by 69% and the area of focal apo(a) accumulation by 97% (Lawn 1996, ibid, table II). It is interesting that Lawn, et al., (1996, ibid) remarked: “But irrespective of the mechanism of action of tamoxifen, we did not expect it to inhibit apo(a) accumulation as well as vascular lesions.”

Note that trifluoperazine, another CaM antagonist, also decreased the rate of lesion formation in rhesus monkeys and in rabbits fed an atherogenic diet (Mohindroo 1997⁴⁶⁴, Mohindroo 1989⁴⁶⁵, Kaul 1987B⁴⁶⁶, Kaul 1987A⁴⁶⁷).

(e) Tenascin-C

(i) Conceptual Background

An increase in β1 integrin-mediated adhesion of monocytes to fibronectin increases TF expression (McGilvray 2002⁴⁶⁸, McGilvray 1997, ibid, Fan 1995, ibid, see also above).

Tenascin-c (TNC) is a large ECM glycoprotein secreted by a variety of cells. TNC decreases β1 integrin-mediated cell adhesion to fibronectin (Probstmeier 1999⁴⁶⁹, Hauzenberger 1999⁴⁷⁰). See also other papers that showed decreased cell adhesion (binding, attachment) to a stratum that includes a mixture of TNC and fibronectin compared fibronectin alone (Huang 2001⁴⁷¹, Pesheva 1994⁴⁷², Bourdon 1989⁴⁷³ Chiquet-Ehrismann 1988⁴⁷⁴). Based on these observations, some papers call TNC an “anti-adhesive” (Doane 2002⁴⁷⁵). Therefore, TNC should decrease TF expression on monocytes/macrophages. Since β1 and TF are expressed in other cell types, it is reasonable to assume that a similar conclusion holds for these cells.

(ii) Prediction 1: Distance

Consider the following sequence of quantitative events.
↑[TNC]→↓[TF in cellⱼ]→↓[TF·plasminogen·fibronectin]→↓TF adhesion curve→↓Skewness of V curve of cellⱼ
Sequence of quantative events 28: Predicted effect of Tenascin-C on skewness of velocity curve.

An increase in TNC concentration in an environment that includes a fibronectin gradient decreases the skewness of the cell velocity curve. What is the effect on the distance traveled by cellᵢ? Consider FIG. 72.

Call the slope of a gradient line “gradient steepness” (see example of a gradient line in section on gradients above). Then, a steeper (gentler) gradient is a gradient with increased (decreased) slope. Consider a steeper fibronectin gradient.
↑[Fn]→↑[TF·plasminogen·fibronectin]→↑TF adhesion curve→↓Skewness of V curve of cellⱼ
Sequence of quantative events 29: Predicted effect of fibronectin gradient steepness on skewness of velocity curve.

A steeper fibronectin gradient can be presented as an increase in skewness (see also examples below).

Consider a fibronectin gradient with certain steepness. Assume that the gradient is associated with skewness and distance illustrated by the coordinates of cell₀. A small increase in concentration of TNC shifts-down the adhesion curve, decreases the skewness of the velocity curve, and increases the distance migrated by the cell. See cell, in figure. A large increase in TNC concentration further decreases skewness. However, the large decrease in skewness decreases the distance migrated by the cell. See cell₂ in figure.

The figure suggests that a biological function of TNC is to increase migration distance in an environment where fibronectin gradient is “too” steep.

(iii) Observations

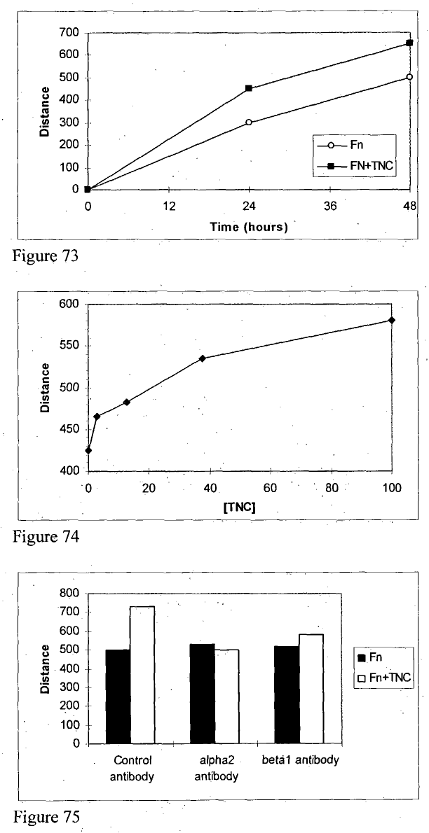

A study (Deryugina 1996⁴⁷⁶) placed spheroids of U251.3 glioma cells on plates coated with fibronectin (10 μg/ml) in the presence or absence of soluble TNC (100 μg/ml). The diameter of the spheroids at the time of plating and following 24-48 hours of migration was measured and compared. FIG. 73 presents the results (Deryugina 1996, ibid, FIG. 8B, distance in elm).

Addition of soluble TNC significantly increased migration distance (p<0.05 at 24 and 48 hours).

The study also measured the effect of a dose increase in TNC concentration on migration. FIG. 74 presents the results (Deryugina 1996, ibid, FIG. 8A, distance in elm).

An increase in TNC concentration, in the range of 3-100 μg/ml, increased migration distance, dose dependently. The observations in Deryugina 1996 (ibid) are consistent with the predicted effect of TNC.

To examine the role of role of α₂β₁ integrin in the effect of TNC on cell migration, the study added soluble TNC (100 mg/ml) in serum-free medium containing a control antibody, or antibodies specific for α₂ or β₁ integrin. FIG. 75 presents the results (Deryugina 1996, ibid, FIG. 9, distance in μm).

Consider the figure in the prediction section. Antibodies against α₂β₁ further decrease skewness. Under a large enough decrease in skewness, the migration distance decreases (see points labeled cell₂ and cell₃ in the figure). The observations are consistent with the predicted effect of the antibodies.

Note: Other studies showed a decrease in migration distance with TNC (Loike 2001, ibid, Andresen 2000⁴⁷⁷). Loike 2001 (ibid) used Matrigel. The relative low concentration of fibronectin in Matrigel positions the fibronectin environment in the increasing section of the figure above. Under such condition, addition of TNC, which decreases skewness, moves the initial point to new points that represent shorter distances, consistent with the reported observations. Andresen 2000 (ibid) added TNC to 50 μg/ml fibronectin, which, according to table I in the paper, seem to produce peak migration distance. In the figure above, if the initial point is positioned at the peak migration distance, addition of TNC, which decreases skewness, moves the initial point to new points that represent shorter distance, also consistent with the reported observations.

(iv) Prediction 2: Co-Localization with Fibronectin

A biological function of TNC is to increase migration distance in an environment where the fibronectin gradient is too steep. Therefore, TNC should co-localize with fibronectin.

(v) Observations

A study (Jones 1997, ibid) collected lung biopsy tissues from 7 patients with progressive pulmonary vascular disease, and stained the tissue for fibronectin and TNC. As expected, the tissues showed intense staining for fibronectin in the immediate periendothelial region (Jones 1997, ibid, FIG. 3A-D). In addition, as expected, the tissues showed intense staining for TNC in the same region (Jones 1997, ibid, FIG. 2D, G, see also table 2).

Notes:

1. Co-localization of fibronectin and TNC was also observed in wounds (Midwood 2002⁴⁷⁸), where TNC appears about 2 hours post injury and continues to increase in concentration before wound contraction. The highest TNC concentration is detected in the margins of the wound bed, the region crossed by macrophages, fibroblasts, and endothelial cells on their way to the wound bed. The localization of TNC in the wound bed margins is consistent with proposed biological function of TNC in increasing migration distance.

2. Co-localization of fibronectin and TNC was also observed in the stroma of tumors.

(vi) Prediction 3: Co-Localization with Macrophages

A steep fibronectin gradient increases macrophage trapping, which occurs at the region of high fibronectin concentration. Since TNC co-localizes with high fibronectin concentration, it should also co-localize with trapped macrophages.

(vii) Observations

A study (Wallner 1999⁴⁷⁹) stained 27 human coronary arteries from 12 patients who underwent heart transplantation for TNC and macrophages. Normal arterial tissue showed no staining for TNC. Atherosclerotic plaque showed co-localized staining for TNC and macrophages (Wallner 1998, ibid, FIG. 2A). According to Wallner, et al., (1998, ibid): “The results of immunostaining data demonstrate a temporospatial correlation between distribution of macrophages and TN-C.” The observations are consistent with the predicted effect of TNC on macrophage migration.

(f) Puberty

(i) Conceptual Background

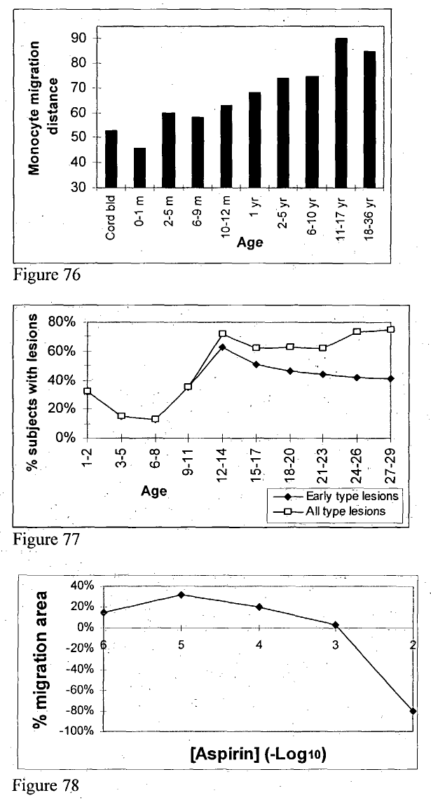

A study (Yegin 1983⁴⁸⁰) used blood samples from subjects at different age groups to measure the distance migrated by monocytes after 90 minutes incubation with the chemotactic factor ZAS. The study measured the distance between the upper surface of the filter in a Boyden chamber and the three most advanced cells in five different fields of each filter. FIG. 76 presents the calculated average monocyte migration distance of different age groups (Yegin 1983, ibid, FIG. 1).

Note the 11-17 year old subjects. Monocytes from 11-17 year old subjects showed the largest migration distance. Moreover, monocytes from 11-17 year old subjects showed a substantially larger migration distance compared to monocytes from 6-10 year old subjects.

An increase in skewness of the velocity curve increases migration distance at all times earlier than the time of equal distance, and decreases distance at all times later than the time of equal distance (see chapter on cell motility, the section entitled “Skewness and distance,” p 155). Assume 90 minutes, the incubation time in Yegin 1983, is less than the time of equal distance. Under such assumption, the increase in distance of the 11-17 year old subjects indicates an increase in skewness of the velocity curve.

(ii) Prediction

Consider the following sequence of quantitative events.
↑Puberty→↑Skewness of monocyte velocity→↑[Lesion]
Sequence of quantative events 30: Predicted effect of puberty onset on rate of lesion formation.

Subjects from the puberty age group should show a higher rate of lesion formation compared to younger subjects.

(iii) Observations

A study (Stary 1989⁴⁸¹) examined the evolution of atherosclerotic lesions in young people by analyzing the coronary arteries and of 565 male and female subjects who died between full-term birth and age 29 years. FIG. 77 presents the observations (Stary 1989, ibid, FIG. 9).

Note the 12-14 year old subjects. According to Stary (1989, ibid): The results suggest “that most subjects destined to have early lesions in the coronary segment under study have developed them by the end of puberty.”. The observations are consistent with the predicted effect of skewness on lesion formation.

Note: According to Stary 1989 (ibid): “Early lesions of the fatty streak type emerged, in most of our subjects, around the age of puberty. The cause of this increased delivery of lipids into the intima remains unexplained. Blood lipids do not increase at that time, and, in fact, serum cholesterol level decreases somewhat at puberty.”

(g) Aspirin (Acetylsalicylic Acid, ASA)

(i) Conceptual Background

(a) Aspirin and TF Transcription In Vitro

A study (Oeth 1995⁴⁸²) treated human monocytes (THP-1) with bacterial lipopolysaccharide (LPS). LPS increased translocation of c-Rel/p65 to the nucleus, binding of c-Rel/p65 heterodimers to a KB site in the TF promoter, and transcription. Presence of aspirin inhibited the LPS-induced translocation of c-Rel/p65. Another study treated isolated human monocytes with LPS in the presence of various concentrations of aspirin. Aspirin dose-dependently inhibited the LPS-induced increase in TF mRNA and protein level (Osnes 1996⁴⁸³, FIG. 3).

Note: Two other studies (Osnes 2000⁴⁸⁴ and Osterud 1992⁴⁸⁵) showed a stimulating effect of aspirin on the LPS-induced increase in TF mRNA. However, the studies used whole blood instead of isolated monocytes. However, isolated monocytes better represent conditions in the intima compared to whole blood.

(b) Aspirin and TF In Vivo

A study (Matetzky 2000⁴⁸⁶) measured the effect of cigarette smoking and aspirin use on tissue factor (TF) expression in atherosclerotic plaque. The study exposed apoE(−/−)mice (n=23) on a high cholesterol diet to cigarette smoke with (n=9) or without (n=14) aspirin treatment (0.5 mg/kg/day). Control mice (n=11) were exposed to filtered room air. After 8 weeks, the aortic root plaque of the mice exposed to smoke was collected and stained for TF. The results showed a significantly smaller TF immunoreactive area in aspirin treated smoker mice compared to untreated smoker mice (6.5±4.5% vs. 14±4%, p=0.002). The area in aspirin treated smoker mice was comparable to the area in non-smoker mice (6.4±3%). TF was largely located in the shoulders of the plaque and in the lipid-rich core. Western blotting showed a 1.3±0.17-fold increase in TF concentration in aspirin treated smokers compared to non-smoker mice, and a 2.3±0.7-fold increase in TF concentration in untreated smokers compared to non-smoker mice.

The study also collected carotid plaques from patients undergoing carotid endarterectomy for symptomatic carotid disease. The plaque was stained for TF. The results showed a significant larger TF staining area in plaque from smokers compared to non-smokers with similar clinical characteristics (8±6% vs. 2.2±2%, p=0.0002). TF co-localized with macrophages in stained plaque. The study also stained for TF in patients treated with aspirin. The portion of patients treated with aspirin in the smoking and non-smoking group was similar. The results showed a significantly smaller TF staining area in plaque from smokers treated with aspirin compared to untreated smokers (4.4±4% vs. 14.5±9%, p=0.0017). Aspirin treated non-smokers also showed smaller TF staining area, however, the difference was not significant, probably because of the small sample size (2.0±2% vs. 3.4±2%, p=0.4).

Notes:

1. The location of TF in shoulders of plaque, lipid-rich core, and macrophages is consistent with the trucking model.

2. The mice were treated with a low dose (0.5 mg/kg/day) of aspirin.

The following sequence of quantitative events presents the relation between aspirin and symbolically.
↑[Aspirin]→↓[TF_{mRNA}]
Sequence of quantative events 31: Predicted effect of aspirin on TF mRNA concentration.

According to the skewed-bell model of cell motility, aspirin should decrease the skewness of the velocity curve and increase the distance traveled by trucking cells. Consider the following section.

(c) Aspirin and Cell Migration In Vitro

Other studies measured the effect of aspirin on cell migration. Brown 1977⁴⁸⁷ used male Wistar rats weighting 220-290 g, the normal body weight for Wistar rats. The study withdrew blood from the rats and isolated leukocytes from the blood. The cells were packed into capillary tubes. Each tube was cut and mounted into migration chambers containing tissue culture media. After 20 hours, cells migrated out from the tube along the floor of the chamber forming a fan-like shape. The relative area of the fan-like shapes represented the rate of cell migration. To test the effect of aspirin on cell migration, the study added various concentrations of aspirin to the culture media. FIG. 78 presents the results (Brown 1977, ibid, FIG. 2). According to the figure, low concentrations of aspirin increased cell migration.

In a follow-up study, Brown 1978⁴⁸⁸ noted that 0.1 and 1 mM aspirin stimulated migration of human lymphocytes in a similar in vitro assay.

Note: Egger 2001⁴⁸⁹ used a special assay to explicitly measure the distance of PMN migration in vitro following treatment with aspirin. The study compared three samples of cells; from atherosclerosis patients treated with aspirin, from patients treated with other medications, mostly, the anticoagulant Phenprocoumon, and from non-atherosclerosis patients. The study did not include a sample of cells from untreated atherosclerosis patients. In addition, the study used whole blood instead of isolated monocytes/macrophages. These issues make the interpretation of the result difficult.

(d) Aspirin and Cell Migration In Vivo

A study (Higgs 1980⁴⁹⁰) implanted subcutaneously polyester sponges impregnated with 2% carrageenin into male rats (150-250 g). The sponges were removed after 24 hours and the total number of leukocytes in the sponges was estimated. To measure the effect of aspirin on the number of migrated leukocytes, the study administered the drug orally at the time of sponge implantation, 5-8 h later, and 3 h before removal. Low doses of aspirin (5-20 mg/kg/day) increased leukocyte migration by 20-70% relative to control values.

Notes:

1. The study observed leukocyte migration out of the tissue and into the sponge. This migration is similar to the migration of trucking cells out of the intima.

2. The decrease in TF expression combined with the increase in cell migration following treatment with aspirin is consistent with the skewed-bell model of cell motility.

(ii) Prediction: Aspirin and Plaque Stability

Consider the following sequence of quantitative events.

1. Macrophages (Mφ)
↑[Aspirin]→↓[TF_{mRNA}]→↓TF_{MΦ} adhesion curve→↓Skewness of V_{B, MΦ} curve→↑TotalD_{B, MΦ}→↓(TotalD_{F, MΦ}−TotalD_{B, MΦ})→↓[Trapped MΦ in intima]
Sequence of quantative events 32: Predicted effect of aspirin on number of trapped macrophages.

Aspirin decreases transcription of TF in intimal macrophages, which shifts-down the adhesion curve, decreases the skewness of the backward velocity curve, resulting in fewer macrophages trapped in the intima.

2. Smooth Muscle Cells (SMC)
↑[Aspirin]→↓[TF _{mRNA}]→↓TF _{SMC} adhesion curve→↓Skewness of V_{SMC} curve→↑ToltalD_{SMC}→↑[SMC in intima]
Sequence of quantative events 33: Predicted effect of aspirin on number of SMC in intima.

Aspirin decreases transcription of TF in media smooth muscle cells, shift-down the SMC adhesion curve, decreases the skewness of the velocity curve directed toward the intima, which increases the number of SMC in the intima.

Similar to a transgenic increase in apo(a) expression (see predictions in the subsection entitled “Transgenic animals” in section on Lp(a) above), treatment with aspirin should decrease the number of macrophages, and increase the number of SMC in the intima.

(iii) Observations

A study (Cyrus 2002⁴⁹¹) fed LDLR(−/−) mice a high fat diet with low dose of aspirin (≈5 mg/kg/day) or placebo for 18 weeks. At the end of the study, the mice were sacrificed, the aortas were harvested, and nuclear extracts were isolated and assayed for NF-κB binding activity. The results showed a significant decrease (34%) in NF-κB binding activity in the aortas of aspirin treated mice compared to controls. The study also examined the number of macrophages and SMC in the aortic vascular lesions. The results showed a decrease in the positive area for macrophages (57%, p<0.05), and an increase in the positive area for SMC (77%, p<0.05) in the aspirin treated mice compared to controls. The results are consistent with the predicted effect of aspirin on cell migration.

(h) CD40

(i) Conceptual Background

CD40, a 50-kDa integral membrane protein, is a member of the tumor necrosis factor receptor (TNF-R) family of proteins. CD40L (CD154, gp39, TBAM), the ligand of CD40, is a 39-kDA member of the TNF family of proteins. After formation of the CD40L·CD40 complex, the CD40-associated factor (CRAF) binds the cytoplasmic tail of CD40 and a signal is produced. CD40 and CD40L are expressed in a variety of cells including T and B-lymphocytes, endothelial cells, fibroblasts, dendritic cells, monocytes, macrophages, and vascular smooth muscle cells.

A study (Schonbeck 2000A⁴⁹²) showed that ligation of CD40 with native CD40L derived from PMA-activated T lymphocytes, or recombinant human CD40L, induced a concentration- and time-dependent transient increase in TF expression on the surface of cultured human vascular SMC. Addition of anti-CD40L mAb blocked the increase in TF cell surface expression. Ligation also induced a concentration- and time-dependent transient increase in total TF concentration and TF procoagulant activity in the treated cells. The study also demonstrated co-localization of TF with CD40 on SMC within atherosclerotic lesions.

An earlier study (Mach 1997⁴⁹³) by the same group showed similar effects of CD40 and CD40L ligation on TF expression in monocytes/macrophages. The following sequence of quantitative events presents the relation between CD40L and CD40 ligation and TF expression symbolically.
↑[CD40L·CD40→↑[TF]
Sequence of quantative events 34: Predicted effect of CD40L and CD40 ligation on TF concentration.

(ii) Prediction: CD40 and Plaque Stability

Consider the following sequence of quantitative events.

1. Macrophages (Mφ)
↑[Anti-CD40L]→↓CD40L·CD40_{MΦ}]→↓[TF_{MΦ}]→↓TF_{M Φ} adhesion curve→↓Skewness of V_{B, MΦ} curve→↑TotalD_{B, MΦ}→↓(TotalD_{F, MΦ}−TotalD_{B, MΦ})→↓[Trapped MΦ in intima] and ↓[LDL in intima]
Sequence of quantative events 35: Predicted effect of anti-CD40L on number of trapped macrophages and LDL concentration in intima.

An anti-CD40L antibody decreases the concentration of the CD40L·CD40 complex on the surface of macrophage, which decreases TF expression in macrophages, resulting in less macrophages trapped in the intima. Moreover, since the macrophages turned foam cells carry the polluting LDL out of the intima, treatment with anti-CD40L should decrease the concentration of LDL in the intima.

2. Smooth Muscle Cells (SMC)
↑[Anti-CD40L]→↓[CD40L·CD40_{SMC}]→↓[TF_{SMC}]→↑TotalD_{SMC}→↓[SMC in intima]
Sequence of quantative events 36: Predicted effect of anti-CD40L on number of SMC in intima.

An anti-CD40L antibody decreases the concentration of the CD40L·CD40 complex on the surface of SMC, which decreases TF expression in SMC, resulting in more SMC in the intima.

Similar to a transgenic increase in apo(a) expression (see above), and treatment with aspirin (see above), treatment with an anti-CD40L antibody should decrease the number of macrophages, and increase the number of SMC in the intima.

(iii) Observations

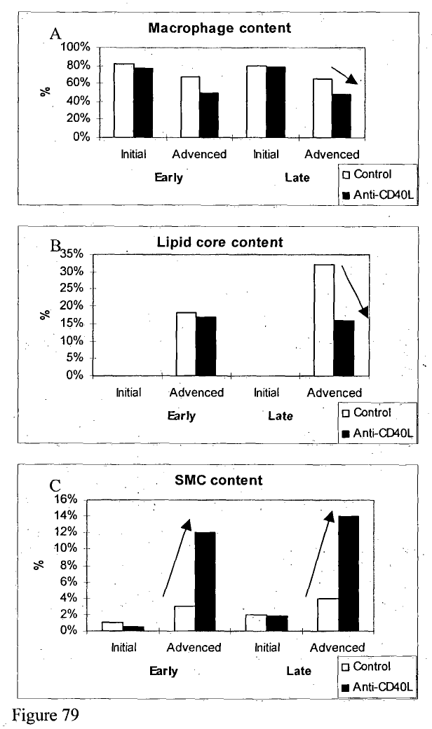

A study (Lutgens 2000⁴⁹⁴) treated apoE(−/−) mice on a chow diet with anti-CD40L antibody or control antibody for 12 weeks. The treatment started early (age 5 weeks) or was delayed until the onset of atherosclerosis (age 17 weeks). The study distinguished between initial lesions defined as fatty streaks containing macrophage-derived foam cells with intracellular lipid accumulation, and advanced lesions defined as lesion containing extracellular lipids, a lipid core and/or fibrous cap. The study examined the content of macrophages, lipid cores, and VSMC in the atherosclerotic lesions. FIG. 79 presents the observations (Lutgens 2000, based on FIG. 1). The delayed anti-CD40L treatment showed a significant decrease in the content of macrophages, a significant decrease in the content of lipid cores, and a significant increase in the content of SMC in advanced lesions. The results are consistent with the predicted effect of anti-CD40L on cell migration.

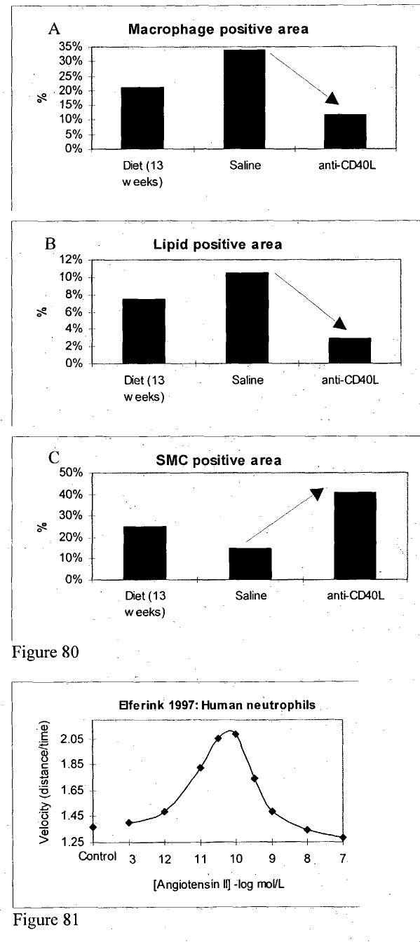

Another study (Schonbeck 2000B⁴⁹⁵) fed LDLR-deficient mice a high-cholesterol diet for 13 weeks, and then for an additional 13 weeks treated the mice with anti-CD40L antibody or saline. During the second 13-week period, mice were continuously fed the high-cholesterol diet. The study examined the areas positive for macrophages, lipid, and VSMC in aortic arch lesions. FIG. 80 presents the observations (Schonbeck 2000B, ibid, FIG. 3). As expected, treatment with anti-CD40L decreased the area positive for macrophages, decreased the area positive for lipids, and increased the area positive for SMC. See also Mach 1998⁴⁹⁶, an earlier study by the same group with similar observations.

Also, as expected, another study showed decreased macrophage content and lipid containing plaque in CD40L(−/−), ApoE(−/−) double transgenic mice compared to ApoE(−/−) single transgenic mice (Lutgens 1999⁴⁹⁷, FIG. 3).

(i) Angiotensin II

(i) Conceptual Background

(a) Introduction

The rennin-angiotensin system (RAS) generates angiotensin II in 2 sequential steps: renin converts angiotensinogen to angiotensin I (Ang I), and the angiotensin-converting enzyme (ACE) converts angiotensin I to angiotensin II (Ang II). ACE also catabolizes other peptides, such as substance P and bradykinin, into inactive metabolites. Smooth muscle cells express ACE. Monocytes show almost no expression of ACE. However, differentiation of monocytes to macrophages results in 5- to 40-fold increase in ACE expression (Viinikainen 2002⁴⁹⁸, Diet 1996⁴⁹⁹, Aschoff 1994⁵⁰⁰, Lazarus 1994⁵⁰¹). Diet 1996 (ibid) also showed that THP-1 cells differentiated into macrophages following treatment with PMA, further increase ACE expression in response to a second treatment with acetylated LDL-C (acLDL). Angiotensin II binds, in humans, two highly specific receptors located on the cell membrane: angiotensin IT type 1 (AT₁), and angiotensin II type 2 (AT₂) (Unger 2002⁵⁰²). Both SMC and macrophages express AT₁.

(b) Angiotensin II and NF-κB

Numerous studies showed activation of NF-κB following treatment with angiotensin II (Tham 2002⁵⁰³, Wolf 2002⁵⁰⁴, Diep 2002⁵⁰⁵, Chen 2002⁵⁰⁶, Theuer 2002⁵⁰⁷, Muller 2000C⁵⁰⁸, Muller 2000B⁵⁰⁹, Muller 2000A⁵¹⁰, Dechend 2001A⁵¹¹, Dechend 2001B⁵¹², Gomez-Garre 2001⁵¹³, Ruiz-Ortega 2001A⁵¹⁴, Ruiz-Ortega 2001B⁵¹⁵, Ruiz-Ortega 2000A⁵¹⁶, Ruiz-Ortega 2000B⁵¹⁷, Brasier 2000⁵¹⁸, Rouet-Benzineb 2000⁵¹⁹, Park 2000⁵²⁰.

(c) Angiotensin II and TF

As expected, studies showed increased TF expression following treatment with angiotensin II.

(d) ACE Inhibitors and NF-κB

A study (Hernandez-Presa 1997⁵²¹) induced accelerated atherosclerosis in femoral arteries of rabbits by endothelial desiccation and an atherogenic diet for 7 days. The atherosclerotic vessels showed an increase in NF-κB activity. Treatment with the ACE inhibitor quinapril decreased the NF-κB activity. Moreover, treatment of cultured monocytes and VSMC with angiotensin II increased NF-κB activation. Pre-incubation with pyrrolidinedithiocarbamate (PDTC), an inhibitor of NF-κB activation, prevented the increase in NF-κB activation. A follow-up study (Hernandez-Presa 1998⁵²²) showed similar effects of quinapril treatment on NF-κB activation.

(e) ACE Inhibitors and TF

(i) In Vitro

A study (Napoleone 2000⁵²³) incubated mononuclear leukocytes from healthy volunteers with endotoxin and the presence and absence of different ACE inhibitors. The ACE inhibitors captopril, idrapril, or fosinopril decreased TF activity in endotoxin-stimulated mononuclear leukocytes in a dose-dependent manner (Napoleone 2000, ibid, FIGS. 1 and 2). The angiotensin II type 1 receptor (AT₁) antagonist losartan caused a similar decrease in TF activity (Napoleone 2000, ibid, FIG. 3). Moreover, captopril also inhibited the increase in TF mRNA in mononuclear leukocytes exposed to endotoxin (Napoleone 2000, ibid, FIG. 4). Finally, captopril, at 20 μg/mL, almost completely inhibited the nuclear translocation of c-Rel/p65, induced by endotoxin treatment (Napoleone 2000, ibid, FIG. 5).

Another study (Nagata 2001⁵²⁴) showed a dose-depended increase in TF antigen and mRNA in monocytes isolated from healthy volunteers following in vitro treatment with angiotensin II (Nagata 2001, FIGS. 2 and 3). The ACE inhibitor captopril and the AT1 antagonist candesartan decreased the level of TF antigen and mRNA in the cultured cells (Nagata 2001, ibid, FIGS. 4 and 5).

(ii) In Vivo-Animal Studies

A study (Zaman 2001⁵²⁵) showed increased TF mRNA in cardiac tissue of obese mice (C57BL/6J ob/ob) relative to lean controls. Treatment of obese mice with the ACE inhibitor temocapril, from 10 to 20 weeks of age, attenuated the increase in TF mRNA (Zaman 2001, ibid, FIGS. 3 and 5).

(iii) In Vivo-Patient Studies

A study (Soejima 1996⁵²⁶) recruited 22 patients 4 weeks after the onset of acute myocardial infarction (AMI). Baseline plasma TF antigen levels were significantly increased compared to controls. Administration of the ACE inhibitor enalapril resulted in a negative effect on the TF antigen level starting from day 3(236±21 at baseline vs. 205±14 on day 3). The decrease became significant on day 28 (169±13). Administration of a placebo to a control group resulted in no significant change in plasma TF antigen level (Soejima 1996, ibid, FIG. 3). Similar observations are reported in Soejima 1999⁵²⁷. Soejima 2001⁵²⁸ also tested the effect of the AT₁ antagonist losartan on AMI patients. The results showed a negative effect on plasma TF antigen levels starting on day 3, which became significant on day 28. The effect of losartan was comparable to enalapril (Soejima 2001, ibid, FIG. 3).

(f) Angiotensin II and Cell Migration

According to the skewed-bell model of cell motility, treatment with angiotensin II should produce a skewed to the right, bell-shaped velocity curve. Consider the following observations.

A study (Elferink 1997⁵²⁹) placed neutrophils, isolated from blood of healthy donors, in the upper compartment of a Boyden chamber. Angiotensin II was placed in the lower compartment, and the cells were allowed to migrate through the filter that separated the compartments. After 35 minutes, the filter was removed, fixed, and stained. The distance the cells traveled into the filter, in μm, was measured according to the leading front technique. FIG. 81 presents the observed effect of angiotensin II concentration on cell velocity (velocity=distance/time) (the figure is based on FIG. 1 in Elferink 1997, ibid, the original figure presents distances instead of velocities).

As expected, the cell velocity curve is a skewed to the right, bell-shaped curve.

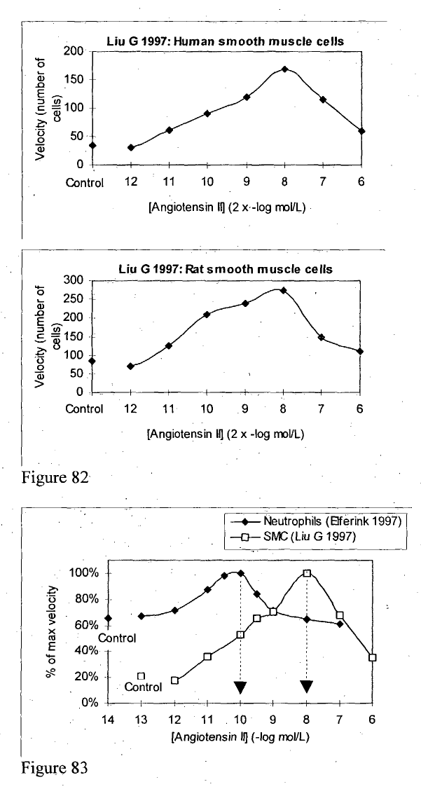

Another study (Liu G 1997⁵³⁰) measured cell migration using Nunc four-well glass culture chambers pre-coated with rat fibronectin (5 μg/mL). Human or rat vascular smooth muscle cells (3×10⁵) were seeded in one corner of the chamber, incubated overnight to allow attachment, and a start line was drawn along the edge of the attached cells. Onto the opposite side of the chamber, the study glued, with preheated (50° C.) 0.5% agarose, an 8-mm² piece of filter paper pre-incubated in 0.1% agarose containing angiotensin II. The cells were incubated for 48 hours. At the end of the incubation, cells were washed, fixed, and stained. Migration was determined by counting the cells across the start line. To minimize cell proliferation, the cells were treated with cytosine. Assume the number of cells across the start line is a linear function of cell velocity, then, FIG. 82 presents the effect of angiotensin II concentration on cell velocity (based on Liu G 1997, ibid, FIG. 1).

As expected, both cell velocity curves are skewed to the right, bell-shaped curves.

Notes:

1. The skewness is more evident in the case of rat SMC.

2. Instead of assuming a linear relation between the number of cells across the start line and cell velocity, a formal model should be presented that derives the relation from more basic elements.

To compare the selective effects of angiotensin II on the two cell types, one needs to present the velocities of both cell types on the same Y-axis. However, the assays in Elferink 1997 (ibid) and Liu G 1997 are different, and therefore, produce results that do not permit such presentation without transformation. FIG. 83 presents the observations of the two studies transformed by calculating, for every angiotensin II concentration, the “% of maximum velocity.”

Neutrophils showed peak velocity at a lower angiotensin II concentration compared to SMC. In terms of skewness, the neutrophil velocity curve shows increased skewness relative to the SMC curve. There are many ways to formally present a difference in skewness (see chapter on cell motility, p 145). One possibility is to assume for the two curves the same “b” and “c” parameters, and a different “a” parameter. In this case, increased skewness is presented with higher “a” values. The following equation summarizes the relation between the “a” values of the two curves.
aₙₑᵤₜᵣₒₚₕᵢₗ=a_{SMC}+a₀, where a₀>0

Since a₀ represents the difference between the two curves, it is independent of any specific angiotensin II concentration; that is, a₀ is the same for all angiotensin II concentrations that specify a certain gradient.

Notes:

1. Assume that a polluted intima shows a gradient of angiotensin II concentrations. Such assumption is consistent with the observed gradual increase in ACE activity in aortas of cholesterol-fed rabbits during the period when no atherosclerotic lesions are observed (Hoshida 1997⁵³¹, FIG. 1). A similar increase in ACE mRNA and protein was observed in atherosclerotic Hamster aortas (Kowala 1998⁵³², see details below). The assumption is also consistent with the observations in a study that examined ACE expression at 37 sites with angioplasty injury caused by percutaneous transluminal coronary angioplasty (PTCA), obtained at autopsy (Ohishi 1997⁵³³) Two months after PTCA, atheromatous plaque at the site of injury showed ACE expression, first in accumulated macrophages, and then in the newly arrived smooth muscle cells. Expression was limited to intermediately differentiated SMC. Highly differentiated SMC in the neointima showed little ACE immunoreactivity. Three months after PTCA, the number of cells with ACE expression decreased. Seven months after PTCA, ACE expression returned to levels comparable to tissue segments without angiographic evidence of restenosis. The observations suggest that migrating macrophages and SMC participate in generating the angiotensin II gradient, while mature, non-migrating SMC, do not.

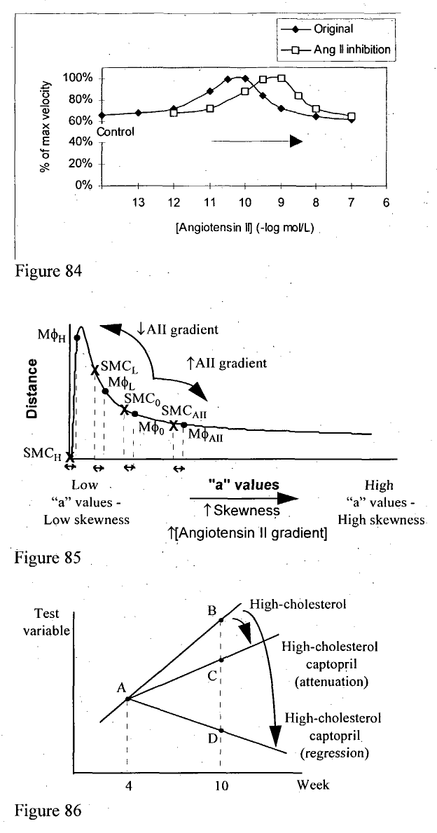

Consider the effect of treatment with a certain concentration of an angiotensin II inhibitor. Assume the inhibitor decreases the local concentration of angiotensin II by a fixed 90%. Consider the location with an original angiotensin II concentration of 10⁻¹⁰, the concentration of peak velocity. According to the figure, the velocity of neutrophils at that location is 100%, or maximum velocity. The effect of the angiotensin II inhibitor is to decrease the angiotensin II concentration to 10⁻¹¹=10⁻¹⁰*10%. According to the figure, the new velocity, the one that corresponds to the new concentration of 10⁻¹¹, is 88% of maximum velocity. Consider the location with the original angiotensin II concentration of 10⁻⁹. The new concentration is 10⁻¹⁰=10⁻⁹*10%, and the new velocity is 100%, or maximum velocity. FIG. 84 presents the effect of treatment with an angiotensin II inhibitor on the velocity curve of neutrophils.

Treatment with the angiotensin IT inhibitor decreases the skewness of the velocity curve, or decreases the value of the “a” parameter. The example demonstrates that any given concentration of an angiotensin II inhibitor is associated with a certain decrease in the value of the “a” parameter.

2. Other treatments that change the angiotensin II gradient have a similar effect on the “a” parameter. For instance, an increase in the oxLDL concentration, which increases the angiotensin gradient (the opposite effect of the angiotensin II inhibitor), increases the value of the “a” parameter.

(g) Angiotensin II and Plaque Stability

Assume the response of macrophages to angiotensin II is similar to the response of neutrophils. Consider FIG. 85.

As noted before, a point on the curve in the figure corresponds to an entire velocity curve in the plane defined by velocity and angiotensin II concentration, where each velocity curve is represented by its skewness and the area under the curve (see chapter on cell motility, p 145). Another difference between the velocity and distance curves relates to angiotensin II. In the velocity plane, a point on the curve associates the local concentration of angiotensin II with cell velocity at that location. In the distance, plane, a point associates an angiotensin II gradient with the distance traveled by the cell in this gradient, at a given time-interval.

The horizontal distance between corresponding M+ and SMC points, such as Mφ₀, SMC₀, or Mφ_{L}, SMC_{L}, marked with two arrows, is equal to the value of a₀ presented in the notes above. Points Mφ₀, SMC₀ represent atherosclerosis. Treatment with a low dose angiotensin II inhibitor increases the “a” values (see notes above), which moves the points to Mφ_{L}, SMC_{L}, representing lower levels of skewness. A high dose moves the points further, to Mφ_{H}, SMC_{H}. In the figure, the low dose increases the distance traveled by macrophages, and decreases the number of cells trapped in the intima. The low dose also increases the distance traveled by the smooth muscle cells, and increases the number of SMC in the intima. The high dose also increases the distance traveled by macrophages. However, unlike the low dose, it decreases the distance traveled by SMC, which should decrease the number of SMC in the intima. Excessive angiotensin II moves the points from Mφ₀, SMC₀ to Mφ_{AII}, SMC_{AII}. The following sequence of quantitative events present similar conclusions:

1. Macrophages (Mφ)
↑[Ang II inhibitor]→↓[Angiotensin II]→↓[TF _{mRNA}]→↓TF _{MΦ} adhesion curve→↓Skewness of V_{B, MΦ} curve→↑TotalD_{B, MΦ}→↓(TotalD_{F, MΦ}−TotalD_{B, MΦ})→↓[MΦ trapped in intima] and ↓[LDL in intima]
Sequence of quantative events 37: Predicted effect of angiotensin II inhibitor on number of trapped macrophages and concentration of LDL in intima.

An angiotensin II inhibitor decreases the concentration of angiotensin II, decreases transcription of TF in intimal macrophages, shifts-down the adhesion curve, decreases skewness of the backward velocity curve, and decreases the number of macrophages trapped in the intima.

2. Smooth Muscle Cells (SMC)

Assume low dose angiotensin JI inhibitor, then,
↑[Ang II inhibitor]→↓[Angiotensin II]→↓[TF _{mRNA}]→↓TF_{SMC} adhesion curve→↓Skewness of V_{SMC} curve→↑TotalD_{SMC}→↑[SMC in intima]
Sequence of quantative events 38: Predicted effect of angiotensin II inhibitor on number of SMC in intima.

An angiotensin II inhibitor decreases the concentration of angiotensin TI, decreases transcription of TF in media smooth muscle cells, shifts-down the SMC adhesion curve, decreases skewness of the velocity curve directed toward the intima, and increases the number of SMC in the intima. Note that the decrease in the number of macrophages and the increase in the number of SMC offset each other with respect to the lesion area. Therefore, a treatment with low dose inhibitor can increase, decrease, or cause no change in the lesion area. However, if the treatment changes the lesion area, the change should be small (see also discussion above on plaque stability).

High dose decreases the total distance traveled by the SMC toward the intima, and decreases the number of these cells in the intima. Note that when both the number of macrophages and the number of SMC decrease in the intima, the lesion area also decreases (see discussion above on plaque stability). Symbolically,
↓[MΦ trapped in intima] AND ↓[SMC in intima]→↓Lesion area
Sequence of quantitative events 39: Predicted effect of number of trapped macrophages and number of SMC in intima-on lesion area.

(ii) Predictions and Observations:

Angiotensin II Infusion/Injection

(a) Animal Studies

(i) Daugherty 2000

A study (Daugherty 2000⁵³⁴) infused angiotensin II (500 or 1,000 ng/min/kg) or vehicle for 1 month via osmotic mini-pumps into mature apoE(−/−) mice. The infused angiotensin II did not change arterial blood pressure, body weight, serum cholesterol concentrations, or the distribution of lipoprotein cholesterol. In the figure above, points SMC_{Athero} and Mφ_{Athero} represent the apoE(−/−) mice before the infusion. An increase in angiotensin II moves the points to SMC_{AII} and Mφ_{AII}, which indicate a decrease in SMC in the intima, and increase in the number of macrophages trapped in the intima.

A study (Allaire 2002⁵³⁵) showed an inverse relation between vascular smooth muscle cell (VSMC) density and formation of abdominal aortic aneurysms (AAA). See also Theocharis 2001⁵³⁶, Raymond 1999A⁵³⁷, and Raymond 1999B⁵³⁸. Therefore, the predicted decrease in SMC in the intima should promote the development of AAA. Consider the following sequence of quantitative events.
↑[Angiotensin II]→ . . . →↓[SMC in intima]→↑[AAA]
Sequence of quantative events 40: Predicted effect of angiotensin II on formation of abdominal aortic aneurisms.

The increase in the number of trapped macrophages increases the rate of lesion formation. Consider the following sequence of quantitative events.
↑[Angiotensin II]→ . . . →↓[MΦ trapped in intima]→↑[Lesion]
Sequence of quantative events 41: Predicted effect of angiotensin II ob rate of lesion formation.

As expected, Daugherty 2000 (ibid) reported that angiotensin II infusion promotes the development of AAA, and increases the rate of atherosclerotic lesion formation in the thoracic aorta.

(ii) Keidar 1999

A study (Keidar 1999⁵³⁹) injected apolipoprotein E deficient mice with angiotensin II (0.1 ml of 10⁻⁷M per mouse, intraperitoneally, once a day for 30 days) or placebo. The angiotensin II injection did not change blood pressure. As expected, the angiotensin II injected mice developed a lesion area of 5,000 μm² with lipid-loaded macrophages, while the placebo-injected mice showed almost no lesion area (Keidar 1999, ibid, FIG. 1).

(iii) Prediction and Observations: ACE Inhibitors and AT₁ Antagonist

(a) Animal Studies

(i) Predictions

Studies of ACE inhibitors and AT₁ antagonists in animals usually use higher doses of the test agent compared to doses used in clinical studies (see details below). In terms of the figure above, points Mφ₀, SMC₀ represent the animal before treatment with the agent. Following treatment, the animal moves to points Mφ_{H}, SMC_{H}, which indicate a decrease in the number of macrophages trapped in the intima, the number of SMC in the intima, and the rate of lesion formation. The improved trucking of LDL also decreases lipid pollution in the intima. Consider the following observations.

(ii) Observations

Warnholtz 1999

Watanabe rabbits show hypercholesterolemia secondary to an LDL-receptor defect. A study (Warnholtz 1999⁵⁴⁰) fed Watanabe rabbits and New Zealand White rabbits chow or a high-cholesterol diet. The Watanabe rabbits on the high-cholesterol diet, Watanabe rabbits on chow, and New Zealand White rabbits, on chow, showed significantly different levels of total plasma cholesterol (1,362±92, 603±45, 32±3 mg/dL, respectively). The study treated the rabbits with the AT₁-receptor antagonist Bay 10-6734 (25 mg/kg/day). The antagonist did not change the cholesterol levels in the hyperlipidemic or control animals. As expected, animals on the high-cholesterol diet treated with the AT₁-receptor antagonist showed decreased fat-stained area in the aorta compared to high-cholesterol fed controls (5.3±1.4% vs. 28.6±7.5%). Also, as expected, histochemical analysis with the monoclonal antibody RAM 11 showed decreased % of macrophage stained area/total plaque cross sectional area in animals treated with the AT₁-receptor antagonist compared to controls (1±0.2% vs. 58.8±15%).

de Nigris 2001

A study (de Nigris 2001⁵⁴¹) treated 2-month-old male apoE(−/−) mice with moderate doses of the ACE inhibitors zofenopril (0.05 or 1 mg/kg/day, N=10 each dose), captopril (5 mg/kg/day, N=10), enalapril (0.5 mg/kg/day, N-8), or placebo, for 29 weeks. Treatment did not change blood pressure, plasma cholesterol or plasma triglyceride. As expected, treatment with zofenopril (both doses) or captopril significantly decreased total lesion area compared to treatment with placebo. However, animals treated with enalapril showed no significant decrease in lesion area compared to placebo. Also as expected, mice treated with zofenopril (1 mg/kg/day) showed a significant decrease in macrophage-derived foam cells staining in the intima compared to placebo treated animals. Finally, also as expected, zofenopril (1 mg/kg/day) treated animals showed a significant decrease in native LDL staining in the intima compared to placebo treated animals.

Keidar 2000

A study (Keidar 2000⁵⁴²) treated apoE(−/−) mice with the ACE inhibitor ramipril (1 mg/kg/day) for 10 weeks. Treatment with the ACE inhibitor did not change blood pressure or plasma cholesterol. As expected, mice treated with the ACE inhibitor showed significantly smaller lesion area compared to placebo treated animals (6,679±978 vs. 25,239±1,899 μm², respectively). Note, that in the same study, mice treated with hydralazine showed a significant decrease in blood pressure with lesion area larger than placebo treated animals (37,165±4,714 vs. 25,239±1,899 μm², respectively). Based on these observations, Keidar, et al., (2000, ibid) concluded: “the anti-atherogenic effect of ramipril in E(O) mice is independent of blood pressure reduction.”

Kowala 1995

A study (Kowala 1995⁵⁴³) treated hamsters on a 4-week high-cholesterol diet with the ACE inhibitor captopril (100 mg/kg/day) for 6 more weeks. The high-cholesterol diet was continued during the treatment with the ACE inhibitors. The statistical tests in the study are somewhat unusual. The study compared observations before treatment with the ACE inhibitor, or week 4, and after treatment with the agent, or week 10. There was no attempt to statistically compare animals treated with the agent to animals on a 10-week high-cholesterol diet only. Consider FIG. 86.

If treatment with captopril regresses the effect of a high-cholesterol diet on the test variable, the value observed on week 10 (point D) will be lower than the value observed on week 4 (point A). However, if the treatment only attenuates the effect of the high-cholesterol diet, the value observed on week 10 (point C) might be higher than the value observed on week 4 (point A). Note that the proposed theory only predicts an attenuation effect. Therefore, point C, which is higher than point A but still lower than point B, the value observed on week 10 on a high-cholesterol diet only, is also consistent with the predicted effect of captopril.

After 6 weeks of treatment with captopril, the animals showed no change in LDL plus VLDL, or total triglyceride levels, and a 24% decrease in HDL compared to levels observed on week 3 and 4. Mean arterial pressure and heart rate showed a small, but significant decrease compared to levels observed on week 3 and 4 (Kowala 1995, ibid, table 2). However, the levels after 6 weeks of treatment with the agent are similar to those observed in animals on a 12-week high-cholesterol diet only (Kowala 1995, ibid, table 1, as mentioned before, the study did not compare statistically the values in table 2 to the values in table 1).

In terms of atherosclerosis, as expected, a 6 week treatment with the ACE inhibitor captopril significantly decreased the number of subendothelial macrophage derived foam cells compared to the levels observed on week 4 (87±9 vs. 52±9, in cell/mm², p<0.05), the average size of a foam cell (113±8 vs. 89±5, in μm², p<0.05), and the area of fatty streak (125±18 vs. 55±12, in. μm2×1,000, p<0.05). The area of extracellular lipid particles showed no significant difference (144±27 vs. 157±36, in μm2×1,000). Under the reasonable assumption that animals on a 10-week high-cholesterol diet only would show a higher area of extracellular lipid particles compared to animals on a 4-week diet, the treatment with captopril, most likely, attenuated the increase in this area (see figure above). The speculated increase in the area of extracellular lipid particles is supported by FIG. 2 in the paper that shows a continued increase in the number of macrophages derived foam cells from week 4 to week 10 in animals on a high-cholesterol diet only. The observations reported in Kowala 1995 (ibid) are consistent with the effect of an ACE inhibitor predicted by the proposed model.

It is amazing to note that in the discussion, Kowala, et al., (1995, ibid) speculate about the existence of a relation between angiotensin II and macrophage backward motility. “In the regression study, ACEI may have decreased the production of arterial AII, which decreased monocyte recruitment to the aorta and increased macrophage mobility (thus promoting the efflux of macrophages from the artery wall). It may explain the reversal of macrophage-foam cell number and also may account for the small size of these cells because delaying the diapedesis of monocytes and promoting efflux of arterial macrophages decreases the residence time and the opportunity for macrophages to accumulate lipid.” (Underline added). However, to the best of my knowledge, these words are the only reference in the literature to such a relation.

Kowala 1998

A study (Kowala 1998, ibid) treated hamsters on a high-cholesterol diet with the ACE inhibitor captopril (100 mg/kg/day), or the HMG-CoA reductase inhibitor pravastatin (34 mg/kg/day), for 8 weeks (see more on HMG-CoA reductase inhibitors, or statins, below).

Treatment with pravastatin decreased plasma total cholesterol (11.8±0.8 vs. 20.0±1.0 mM, p<0.025), VLDL+LDL cholesterol (8.8±0.7 vs. 17.9±1.0 mM, p<0.025), total triglycerides (4.8±0.3 vs. 29.1±3.4 mM, p<0.001), and increased HDL cholesterol (3.0±0.1 vs. 1.8±0.02 mM, p<0.001) compared to controls. In contrast, treatment with captopril did not change plasma lipids. Treatment with captopril decreased mean arterial pressure (110±5 vs. 139±5 mm Hg, p<0.025) and heart rate (348±6 vs. 376±6 beats/min, p<0.025) compared to controls. In contrast, treatment with pravastatin did not change mean arterial pressure or heart rate.

In terms of atherosclerosis, treatment with pravastatin decreased the cell size of macrophage derived foam cells (103±5 vs. 130±5 μm², p<0.045) but did not change the subendothelial number of these cells in the aortic arch. Treatment with captopril had the opposite effect. Captopril did not change the cell size of macrophage derived foam cells, but decreased the subendothelial number of these cells in the aortic arch (108±10 vs. 164±19 cells/mm², p<0.045). Both pravastatin and captopril decreased the fatty streak area (31% p=0.092 and 35% p=0.056, respectively), although the statistical significance was somewhat higher than 5%. As expected, treatment with the ACE inhibitor decreased the subendothelial number of macrophage derived foam cells and rate of lesion formation.

Note that Captopril increased macrophage migration distance without changing the cell lipid content. Also note that Pravastatin did not change the number of macrophages in the lesion. The result seems inconsistent with the effects of statin described below. However, it can be explained as movement to a new point in the skewness figure on other side of the peak that represents a similar migration distance as the original point. In such a case, the similar number of macrophages and the decreased number of SMC should result in smaller lesions.

Napoli 1999

A study (Napoli 1999⁵⁴⁴) treated Watanabe rabbits with the ACE inhibitor zofenopril (0.5 mg/kg/day), or placebo, for 6 weeks. Treatment with zofenopril decreased the aortic and common carotid corrected cumulative lesion area by 34% and 39%, respectively (p<0.05), the intimal presence of macrophage derived foam cells (p<0.05), and native LDL (p<0.01), compared to the placebo-treated animals. The observations are consistent with the effect of an ACE inhibitor predicted by the proposed model.

(iii) Summary

The following table summarizes the observations reported in the animal studies above. The word “consistent,” next to a quantitative event, marks an event that is consistent with the predicted effect of the treatment according to the suggested model.

TABLE 12
Summary of observed effects of angiotensin II, AT₁-receptor antagonist, or ACE
inhibitor treatments on rate of lesion formation, and macrophage, SMC, and lipid content.

| | | Treatment | | | | |
|---|---|---|---|---|---|---|
| Study | Animals | (dose) | Lesion | Mφ | SMC | Lipids |
| Daugherty | ApoE(−/−) | Ang II | ↑ | | ↓ | |
| 2000 | mice | | consistent | | consistent | |
| | | | | | (via | |
| | | | | | AAA) | |
| Keidar | ApoE(−/−) | Ang II | ↑ | | | |
| 1999 | mice | | consistent | | | |
| Warnholtz | Watanabe | AT₁- | | ↓ | | ↓ |
| 1999 | rabbits | receptor | | consistent | | consistent |
| | | antagonist | | | | |
| | | Bay 10-6734 | | | | |
| | | (25 mg/kg/day) | | | | |
| de Nigris | ApoE(−/−) | ACE | ↓ | ↓ | | ↓ |
| 2001 | mice | inhibitor | consistent | consistent | | consistent |
| | | Zofenopril | ↓ | | | |
| | | (0.05 or 1 mg/kg/day) | consistent | | | |
| | | Captopril | NC | | | |
| | | (5 mg/kg/day) | ? | | | |
| | | Enalapril | | | | |
| | | (0.5 mg/kg/day | | | | |
| Keidar | ApoE(−/−) | ACE | ↓ | | | |
| 2000 | mice | inhibitor | Consistent | | | |
| | | Ramipril | | | | |
| | | (1 mg/kg/day) | | | | |
| Kowala | Hamsters | ACE | ↓ | ↓ | | ↓ |
| 1995 | | inhibitor | consistent | consistent | | consistent |
| | | Captopril | | | | |
| | | (100 mg/kg/day) | | | | |
| Kowala | Hamsters | ACE | ↓ | ↓ | | |
| 1998 | | inhibitor | consistent | consistent | | |
| | | Captopril | | | | |
| | | (100 mg/kg/day) | | | | |
| Napoli | Watanabe | ACE | ↓ | ↓ | | ↓ |
| 1999 | rabbits | inhibitor | consistent | consistent | | consistent |
| | | Zofenopril | | | | |
| | | (0.5 mg/kg/day) | | | | |

(b) Clinical Studies

(i) Predictions

Clinical studies of ACE inhibitors and AT₁ antagonists usually use lower doses of the test agent compared to animal studies. Compare the dose of ramipril, 10 mg/day in the HOPE study with patients (see below), and 1 mg/kg/day in the Keidar 2000 (ibid) study with apoE(−/−) mice (see above). Assuming an average body weight of 70 kg, the dose in the patient study is 10/70=0.14 mg/kg/day, more than 7-fold lower than the dose in the animal study. In terms of the figure above, points Mφ₀, SMC₀ represent the patient before treatment with the agent. Following treatment, the patient moves to points Mφ_{L}, SMC_{L} which indicate an increase in plaque stability with no change, or a small change in plaque size. The increase in plaque stability decreases the probability of plaque rupture and likelihood of a cardiovascular event. Consider the following observations.

(ii) Observations

Cardiovascular Events: HOPE Study

A study (Yusuf 2000⁵⁴⁵) randomly assigned 9,297 high-risk patients, 55 years of age or older, with evidence of vascular disease or diabetes, one other cardiovascular risk factor, and no evidence of low ejection fraction or heart failure, to receive ramipril (10 mg/day), or placebo. Average follow up was 4.5 years. The patients treated with ramipril showed a decreased rate of death from cardiovascular causes (6.1% vs. 8.1%, RR=0.74, p<0.001), myocardial infarction (9.9% vs. 12.3%, RR=0.80, p<0.001), stroke (3.4% vs. 4.9% t, RR=0.68, p<0.001), death from any cause (10.4% vs. 12.2%, RR=0.84, p=0.005), revascularization procedures (16.3% vs. 18.8%, RR=0.85, p<0.001), cardiac arrest (0.8% vs. 1.3%, RR=0.62, p=0.02), heart failure (9.1% t vs. 11.6% t, RR=0.77, p<0.001), and complications related to diabetes (6.4% vs. 7.6%, KR=0.84, p=0.03). The beneficial effect of ramipril was observed in all subgroups examined, such as, women, patients with low ejection fraction, hypertension, established vascular disease, and diabetes. The effect was independent of the decrease in blood pressure, and of other medications taken, such as aspirin, diuretics, beta-blockers, or calcium-channel blockers. The observed effect of the ACE inhibitor on the rate of cardiovascular events is consistent with the effect predicted by the proposed model.

Plaque Size: PART-2, SCAT, SECURE

A study (MacMahon 2000⁵⁴⁶, PART-2), assigned 617 patients, in equal proportions, to receive the ACE inhibitor ramipril (5 or 10 mg/day), or placebo. Average follow up was 4 years. The study assessed carotid atherosclerosis by B-mode ultrasound at baseline, two years, and four years. The results showed no significant difference between groups in the changes in thickness of the common carotid artery wall, or carotid plaque height. According to MacMahon, et al., (2000, ibid) “These negative trial results in humans contrast with the evidence of marked anti-atherosclerotic and anti-proliferative effects of very high-dose ACE inhibition in studies of diet- or endothelial injury-induced atherosclerosis in animals. These observations raise doubts about the value of some animal models of atherosclerosis for the investigation of drug effect and the use of drug doses in experimental studies so far outside the range of the typically used in humans.” . . . “However, it is also possible that there are other mechanisms by which ACE inhibitors might alter coronary risk, including reversal of endothelial dysfunction, leading, perhaps, to increased plaque stability and decreased risk of plaque rupture. Further research on the mechanisms of benefit from ACE inhibition is required.”

A study (Teo 2000⁵⁴⁷, SCAT) assigned 460 patients to receive the ACE inhibitor enalapril (5 mg/day) or placebo. Average follow up was 4 years. The study assessed atherosclerosis in coronary arteries by quantitative coronary angiography (QCA) at baseline and closeout, 3 to 5 years later. The results showed no significant difference between groups in changes in mean absolute diameter, minimum absolute diameter, and percent diameter stenosis. According to Teo, et al., (2000, ibid): “Potential mechanisms of the benefit of ACE inhibition include normalization of endothelial dysfunction and plaque formation and stabilization. These effects which are not easily detected by QCA analysis may have been operative in large trials demonstrating clinical benefits.”

A sub-study of HOPE (Lonn 2001⁵⁴⁸, SECURE), assigned 732 patients to receive the ACE inhibitor ramipril (2.5 or 10 mg/day) or placebo. Average follow up was 4.5 years. The study assessed atherosclerosis progression by B-mode carotid ultrasound. The results showed a significant decrease in the progression slope of the mean maximum carotid intimal medial thickness (IMT) in the group treated with ramipril (10 mg/day) compared to the group treated with placebo (0.0137±0.0024 vs. 0.0217±0.0027 mm/year, p=0.0033, p=0.037 after adjustment for blood pressure).

According to a recent review (Halkin 2002⁵⁴⁹): in the SECURE study, “At 4.5 years, ramipril decreased progression of carotid intima media thickness by 0.008 mm per year. Although the difference was statistically significant, it is unlikely that this small effect on atherosclerotic lesion burden explains the reduction in clinical event rates found in the HOPE study. . . . As there is insufficient evidence demonstrating that ACE inhibitors have a major effect on plaque mass or restenosis in humans, the clinical benefits afforded by ACE inhibition cannot be ascribed to the regression of atherosclerotic lesions. The discrepancy between findings in animal and human studies remains to be explained, although it may be the result of dosing (larger doses used in animals) or methodology (the sensitivity of ultrasonography and angiography is limited in comparison with pathologic evaluation performed in animals). Alternatively, it may reflect differences in the pathophysiology of human and animal atherosclerosis.”

The observed small to no effect of ACE inhibition on plaque size is consistent with the effect of ACE inhibition predicted by the proposed model.

(j) HMG-CoA Reductase Inhibitors (Statins)

(i) Conceptual Background

(a) Statins and Signal Intensity

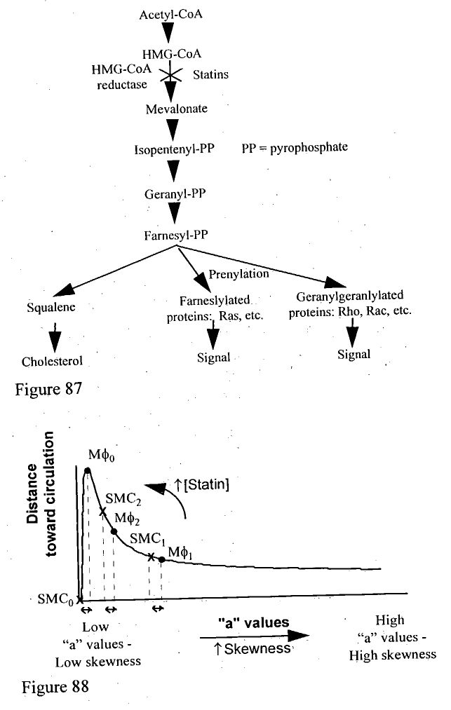

FIG. 87 presents the cholesterol synthesis pathway.

Inhibition of 3-hydroxy-3-methylglutaryl coenzyme A reductase (HMG-CoA) decreases the intensity of the signal produced by members of the Ras and Rho GTPase family of proteins (Takemoto 2001⁵⁵⁰). This effect is called the pleiotropic effect of statins. On the relation between statins and signal intensity, see also Dechend 2001B (ibid).

A dominant negative mutant of Rac decreased NF-κB activation in THP-1 monocytes (Reyes-Reyes 2001⁵⁵¹). In the same cell type, a dominant negative mutant of Ras, or Raf1, inhibited the LPS increase in Egr-1 expression (Guha 2001⁵⁵²). Increased availability of prenylated Rho-A significantly increased the positive effect of angiotensin II (Ang II), hyperglycemia, and advanced glycosylation end products (AGEs) on NF-κB activation in vascular smooth muscle cells (Golovchenko 2000⁵⁵³). The positive effect of Rho on NF-κB activation was also observed in other cell types (Montaner 1999⁵⁵⁴, Montaner 1998⁵⁵⁵).

NF-κB and Egr-1 increase TF transcription. Therefore, statins should decrease TF transcription. Consider the following sections.

(b) Statins and NF-κB Activation

As expected from the effect of statins on signal intensity, statins inhibited NF-κB activation in a variety of cells and tissues. See, for instance, the effect of simvastatin in peripheral mononuclear (PMN) cells and lesions (Hernandez-Presa 2003⁵⁵⁶), cerivastatin, fluvastatin, and pitavastatin in human kidney 293 T-cells (Inoue 2002⁵⁵⁷), pravastatin in isolated human monocytes (Zelvyte 2002⁵⁵⁸), cerivastatin in tissue extracts of left ventricle (Dechend 2001A, ibid), mevastatin in EC (Rasmussen 2001⁵⁵⁹), simvastatin in THP-1 monocytes. (Teupser 2001⁵⁶⁰), atorvastatin in VSMC and U937 monocytes (Ortego 1999⁵⁶¹), and atorvastatin in aorta, liver, lesions, and VSMC (Bustos 1998⁵⁶²).

(c) Statins and TF Expression

As expected from the effect of statins on signal intensity, statins also decreased TF mRNA and protein concentration. See, for instance, the negative effect of cerivastatin, and pravastatin in isolated peripheral blood monocytes (Nagata 2002⁵⁶³, the study also showed decreased Rho by pravastatin), simvastatin in isolated monocytes (Ferro 2000⁵⁶⁴), fluvastatin, and simvastatin in macrophages (Colli 1997⁵⁶⁵, pravastatin showed no effect), fluvastatin in lesions (Baetta 2002⁵⁶⁶).

(ii) Predictions: Statins and Plaque Stability

Consider FIG. 88.

Call the signal produced by Ras, Rho, or Rac, the “triple R” signal. A point on the curve in the figure corresponds to an entire velocity curve in the plane defined by velocity and the intensity of the triple R signal, where each velocity curve is represented by its skewness and the area under the curve (see chapter on cell motility, p 145). Another difference between the velocity and distance curves relates to the triple R signal. In the velocity plane, a point on the curve associates local intensity of the triple R signal with cell velocity at that location. In the distance plane, a point associates a gradient of triple R intensities with distance traveled by the cell in this gradient, at a given time interval.

The horizontal distance between corresponding M4 and SMC points, such as Mφ₀, SMC₀, or Mφ₁, SMC₁, marked with two headed arrows, is equal to the value of a₀ (see above). Points Mφ₀, SMC₀ represent atherosclerosis. Treatment with a low dose statin increases the “a” values, which moves the points to Mφ₂, SMC₂, representing lower levels of skewness. In the figure, the treatment increases the distance traveled by macrophages, and decreases the number of these cells trapped in the intima. The treatment also increases the distance traveled by smooth muscle cells, and increases the number of SMC in the intima.

The following sequence of quantitative events present similar conclusions:

1. Macrophages (Mφ)
↑[Statin]→↓[Triple R single]→↓[TF_{mRNA}]→↓[TF_{mΦ} adhesion curve→↓Skewness of V_{B, MΦ} curve→↑TotalD_{B, MΦ}→↓(TotalD_{F, MΦ}−TotalD_{B, MΦ})→↓[MΦ trapped in intima] and ↓[LDL in intima]
Sequence of quantative events 42: Predicted effect of statin on number of trspped macrophages and LDL concentration in intima.

Treatment with a statin decreases the intensity of the triple R signal, decreases transcription of TF in intimal macrophages, shifts-down the adhesion curve, decreases skewness of the backward velocity curve, and decreases the number of macrophages trapped in the intima.

2. Smooth Muscle Cells (SMC)

Assume low dose angiotensin II inhibitor, then,
↑[Statin]→↓[Triple R signal]→↓[TF_{mRNA}]→↓TF_{SMC} adhesion curve→↓Skewness of V_{SMC} curve→↑TotalD_{SMC}→↑[SMC in intima]
Sequence of quantative events 43: Predicted effect of statin on number of SMC in intima.

Treatment with statin decreases the intensity of the triple R signal, decreases transcription of TF in media smooth muscle cells, shifts-down the SMC adhesion curve, decreases skewness of the velocity curve directed toward the intima, and increases the number of SMC in the intima. Note that the decrease in the number of macrophages and the increase in the number of SMC offset each other with respect to the lesion area. Therefore, the treatment can increase, decrease, or cause no change in the lesion area. However, if the treatment changes the lesion area, the change should be small (see also discussion above on plaque stability).

(iii) Observations

(a) Sukhova 2002

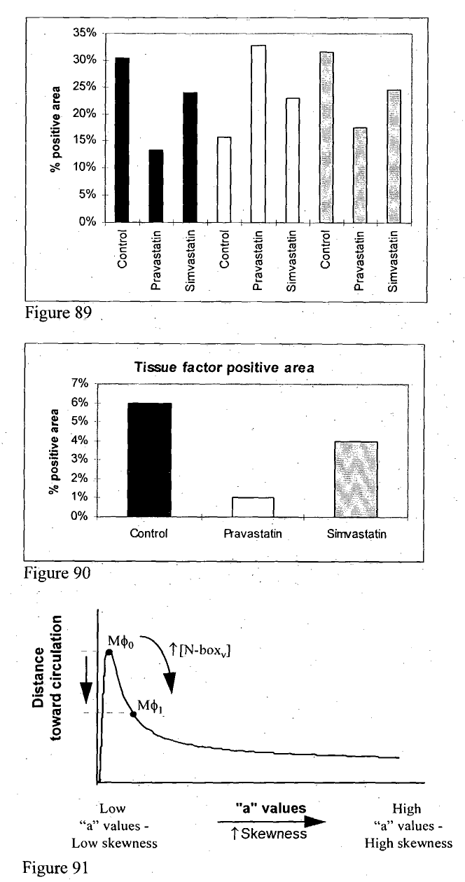

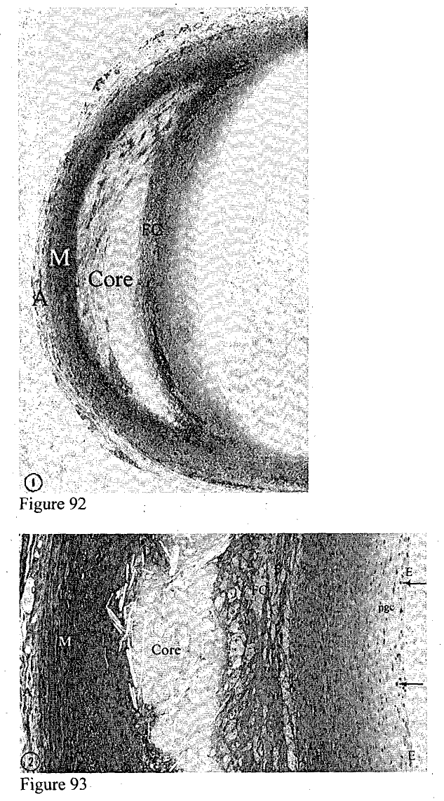

A study (Sukhova 2002⁵⁶⁷) fed adult male cynomolgus monkeys an atherogenic diet while receiving pravastatin (40 mg/kg/day), simvastatin (20 mg/kg/day), or no treatment (control). The study extended over 12 months and included 12 monkeys per group. To eliminate the effect of plasma cholesterol, the study adjusted the dietary cholesterol such that plasma cholesterol levels were equal among groups. At the end of the study abdominal aortas were isolated, stained, and measured. The results showed no difference in plaque size, expressed as intimal area, medial area, or intima/media ratio, among groups. FIG. 89 presents the effect of treatment on areas stained positive for macrophages, SMC, and lipid (Sukhova 2002, ibid, based on FIG. 1). As expected, the number of macrophages decreased, the number of smooth muscle cells increased, and the content of lipids decreased.

The study also measured TF expression in the atheroma of the monkeys. FIG. 90 presents the results (Sukhova 2002, ibid, based on FIG. 3). Consistent with the proposed model, a larger effect on TF expression resulted in a larger effect on cell number and lipid content. Pravastatin decreased TF expression more than simvastatin. As a result, treatment with pravastatin decreased the number of macrophages, increased the number of SMC, and decreased lipid content, more than treatment with simvastatin.

Another study (Aikawa 2001⁵⁶⁸) showed a decrease in the area positive for macrophages in lesions of Watanabe heritable hyperlipidemic rabbits following treatment with cerivastatin (Aikawa 2001, ibid, FIG. 2A). The study also showed a decrease in the area positive for TF in the intima of treated rabbits (Aikawa 2001, ibid, FIG. 4B).

Another study (Baetta 2002, ibid) showed a decrease in the area positive for macrophages, and the area positive for TF, in lesions of New Zealand male rabbits on a cholesterol-rich diet, following treatment with fluvastatin compared to untreated rabbits. Fluvastatin did not change plasma cholesterol level. Double staining with RAM11, a marker for macrophages, and PCNA, a marker of cell proliferation, showed no difference between groups. Total PCNA in the lesion was also similar between groups. Staining with TUNEL, a marker of apoptosis showed little staining with no difference between groups. These results indicated that the effect of fluvastatin on the number of macrophages present in the lesion is not mediated through cell proliferation or apoptosis.

See also a recent review on the relation between statins and plaque stabilization (Libby 2002⁵⁶⁹).

(k) Other Consistent Observations

Other observations fit the same patterns illustrated above. Consider the following examples.

(i) Smoking

A study (Holschermann 1999⁵⁷⁰) showed increased NF-κB activation and TF transcription in monocytes isolated from smoking compared to non-smoking women. Another study (Matetzky 2000, ibid) showed increased TF expression in plaque of apoE-deficient mice exposed to cigarette smoke compared to mice exposed to filtered room air. The increase in TF transcription increases the skewness of the backward velocity curve, and increases the rate of lesion formation. Therefore, smoking should be associated with increased rate of cardiovascular disease. As expected, several studies showed a positive relation between smoking and cardiovascular disease (Simons 2003⁵⁷¹, Jee 1999⁵⁷², Kawachi 1999⁵⁷³, Iribarren 1999⁵⁷⁴, He J 1999⁵⁷⁵, Ockene 1997⁵⁷⁶).

(ii) Red Wine

A study (Blanco-Colio 2000⁵⁷⁷) showed increased NF-κB activation in peripheral blood mononuclear cells isolated from subjects after a fat-rich breakfast. Red wine intake prevented the increase in NF-κB activity. A decrease in NF-κB activity decreases the skewness of the backward velocity curve, and therefore, protects against atherosclerosis. Therefore, red wine intake should show a protective effect against cardiovascular disease. As expected, several epidemiological studies demonstrated the protective effect of red wine intake (see recent reviews, de Gaetano 2001⁵⁷⁸, Rotondo 2001⁵⁷⁹, Sato 2002⁵⁸⁰, Wollin 2001⁵⁸¹).

(iii) ApoE

Similar to apoAI, apolipoprotein E (apoE) increases cholesterol efflux from lipid-loaded cells (Langer 2000⁵⁸², Mazzone 1994⁵⁸³, Huang 1994⁵⁸⁴). Cholesterol efflux decreases skewness of the forward and backward velocity curves (see section on apoAI). The decrease in skewness should decrease the number of macrophages and increase the number of SMC in the intima. As expected, a study (Tsukamoto 1999⁵⁸⁵) showed increased plaque stability in apoE-deficient mice on chow diet with hepatic expression of a human apoE3 transgene.

(iv) NF-κB

A study (Wilson SH 2002⁵⁸⁶) showed increased activation of NF-κB in plaque from patients with unstable angina pectoris (UAP) compared to patients with stable angina pectoris (SAP). Increased activation of NF-κB increased the skewness of the backward velocity curves, which decreases plaque stability.

(v) Tissue Factor

Tissue factor (TF) propels backward migration of lipid-loaded macrophages and smooth muscle cells. TF also propels endothelial cells in angiogenesis. Therefore, Mφ, SMC, and EC in atherosclerotic plaque should show an increase in TF mRNA and activity. As expected, several studies observed an increase in TF mRNA and activity in intimal Mφ, intimal and medial SMC, and EC in microvessels in atherosclerotic plaque (Westmuckett 2000⁵⁸⁷, Crawley 2000⁵⁸⁸ Kaikita 1999⁵⁸⁹, Hatakeyama 1997⁵⁹⁰, Kato 1996⁵⁹¹, Sueishi 1995⁵⁹², Landers 1994⁵⁹³, Wilcox 1989⁵⁹⁴). See also several recent reviews on TF and atherosclerosis (Moons 2002⁵⁹⁵, Tremoli 1999⁵⁹⁶, Taubman 1997⁵⁹⁷, Osterud 1998⁵⁹⁸, Osterud 1997⁵⁹⁹).

Migrating SMC are of an immature phenotype. As expected, a study (Hatakeyama 1998⁶⁰⁰) also showed that following balloon injury, intimal smooth muscle cells positive for TF are of an immature phenotype. In addition, as expected, the study showed that after balloon injury, TF protein and mRNA are rapidly induced in SMC positioned closely underneath the internal elastic lamina.

Another study (Aikawa 1999⁶⁰¹) fed New Zealand White male rabbits a high-cholesterol diet for 4 months. Balloon injury was performed 1 week after initiation of the diet. At the end of the 4 months, a group of rabbits (Baseline) were killed and their aortas were stained for TF. The study divided the remaining rabbits into two groups. The first was continued on the high-cholesterol diet (High) and the second was fed a low-cholesterol diet (Low). Both groups received their respective diets for 16 months. At the end of the 16 months, the rabbits were killed and their aortas were stained for TF. The results showed a decrease in the area positive for TF in both High and Low groups relative to the Baseline group (Aikawa 1999, ibid, FIG. 5). However, the Low group showed a larger decrease in the area positive for TF (pa 0.001 relative to Baseline and High). A cholesterol intake decreases the skewness of the backward velocity curve, which decreases the number of lipid-loaded macrophages trapped in the intima, and therefore, the concentration of TF in the plaque of the Low rabbits.

c) Microcompetition with Foreign DNA and Atherosclerosis

(1) Conceptual Background

(a) Viruses in Monocytes-Turned Macrophages

The subendothelial environment stimulates viral gene expression and replication of latent viruses in monocytes-turned macrophages. Consider the following observations.

Cytomegalovirus (CMV) is a GABP virus. Circulating monocytes are nonpermissive for CMV replication. Monocytes showed no expression of viral gene products even when cells harbor a viral genome (Taylor-Wiedeman 1994⁶⁰²) In monocytes, the virus is in a latent state. Viral replication is dependent on expression of viral immediate-early (1E) gene products controlled by the major immediate-early promoter (MIEP). A study (Guetta 1997⁶⁰³) transfected HL-60, promyelocytic cells that can differentiate into macrophages, with MIEP-CAT, a plasmid that expresses the reporter gene CAT under the control of CMV MIEP. Co-culture of MIEP-CAT-transfected cells with endothelial cells (EC) increased CAT activity 1.7-fold over baseline activity in non co-cultured HL-60 cells (Guetta 1997, ibid, FIG. 1A). Co-culture of MIEP-CAT-transfected cells with smooth muscle cells increased CAT activity 4.5-fold over baseline (Guetta 1997, ibid, FIG. 1B). Treatment with 50 to 200 μg/mL oxLDL activated MIEP in a concentration dependent manner (Guetta 1997, ibid, FIG. 2.). A 2.0-fold increase was the largest observed effect of oxLDL (Guetta 1997, ibid, FIG. 1C). Co-culture with EC plus oxLDL resulted in a 7.1-fold increase over baseline, larger than the two separate effects. Based on these results, Guetta, et al., (1997, ibid) concluded that exposure of monocytes-turned macrophages to EC, SMC, and oxLDL in the subendothelial space favors transactivation of latent CMV.

When cerulenin, an inhibitor of fatty acid biosynthesis, was added to mouse fibroblasts infected with Moloney murine leukemia virus (MMuLV), virus production was drastically decreased (Ikuta 1986B⁶⁰⁴, Katoh 1986⁶⁰⁵). Cerulenin also inhibited Rous sarcoma virus (RSV) production in chick embryo fibroblasts (Goldfine 1978⁶⁰⁶).

Following entry into the subendothelial space, monocytes differentiate into macrophages. Monocyte differentiation transactivated the human CMV IE gene (Taylor-Wiedeman 1994, ibid), and, in some cases, produced productive human CMV infection (Ibanez 1991⁶⁰⁷, Lathey 1991⁶⁰⁸). Similarly, differentiation of THP-1 pre-monocytes (Weinshenker 1988⁶⁰⁹), and T2 teratocarcinoma cells (Gonczol 1984⁶¹⁰), also induced human CMV replication.

EC, SMC, and oxLDL in the subendothelial space stimulate viral gene expression and viral replication in macrophages that harbor latent GABP viruses. The increase in the number of viral N-boxes intensifies microcompetition with cellular genes for GABP. Therefore, entry to the subendothelial space intensifies microcompetition for GABP in monocyte-turned macrophages.

(b) Viruses in Smooth Muscle Cells

SMCs are permissive to CMV (Zhou Y F 1999⁶¹¹, Zhou 1996⁶¹², Tumilowicz 1985⁶¹³, Melnick 1983⁶¹⁴) and HSV (Benditt 1983⁶¹⁵). Monocytes infected with CMV can transmit the virus to neighboring smooth muscle cells (Guetta 1997, ibid).

(2) Excessive Skewness and Fibrous Cap

Consider an area in the intima polluted with LDL. The LDL attracts monocytes. Assume the monocytes are latently infected with a GABP virus. What is the effect of the infection on the monocyte/macrophage migration? Some of the LDL particles in the intima cross the intimal elastic lamina and end up in medial SMC. The oxLDL in medial SMC and the macrophages in the SMC environment induce SMC migration towards the intima. Assume that either infected monocytes transmit the GABP virus to medial SMC, or that both monocytes and medial SMC harbor a latent GABP virus. What is the effect of the infection on SMC migration?

Note: The macrophages can induce SMC migration, by, for instance, increasing Lp(a) concentration in the polluted area (see above).

(a) Effect on Monocytes/Macrophages Migration

(i) Prediction: Mφ Superficial Stop

The subendothelial environment stimulates viral gene expression and replication in the infected monocyte-derived macrophages. The increase in the number of viral N-boxes intensifies microcompetition for GABP. CD18 is a GABP suppressed gene (see chapter on transefficiency, p 140). Therefore, the intensified microcompetition for GABP increases CD18 transcription, shifts-up the adhesion curve, and increases the skewness of the CD18 propelled forward velocity curve. Consider the following sequence of quantitative events. [symbol]
[N-boxᵥ]_{MΦ}→↓[p300·GABP·N-box_{CD18}]→↑[mRNA_{CD18}]→↑Adhesion curve→↑Skewness of V_{F} curve→↓TotalD_{F}→↓Intimal depth at rest AND ↑[ECM bound oxLDL deep in the intima]
Sequence of quantative events 44: Predicted effect of foreign N-boxes on intimal depth at rest and concentration of ECM bound ox LDL deep in the intima.

An increase in the number of viral N-boxes increases skewness of the forward velocity curve, decreases total forward distance, and decreases intimal depth at rest. An infection with a GABP virus produces a superficial stop. The superficial stop diminishes clearance of ECM bound oxLDL deep in the intima.

CD49d (α4 integrin) is also a GABP suppressed gene (see chapter on transefficiency, p 140). Therefore, a similar sequence of quantitative events holds for CD49d.

Note that backward propulsion is coordinated with forward propulsion. The decrease in TotalD_{F} equally decreases the corresponding TotalD_{B} (see above). Therefore, the decrease in total forward distance does not increase the number of macrophages trapped in the intima (see more in next section).

(ii) Prediction: Mφ Trapping

The subendothelial environment stimulates viral gene expression and replication in infected macrophages which intensifies microcompetition for GABP. TF is a GABP suppressed gene (see Appendix). Therefore, the intensified microcompetition increases TF transcription. Tenascin-C (TNC) is also a GABP stimulated gene (Shirasaki 1999⁶¹⁶). INC decreases TF transcription (see above). Therefore, microcompetition with the GABP virus decreases TNC transcription, which further increases TF transcription. The increase in TF transcription shifts-up the adhesion curve, and increases the skewness of the TF propelled backward velocity curve. Consider the following sequence of quantitative events.

An increase in the number of viral N-boxes increases skewness of the TF propelled backward velocity curve, decreases the total distance traveled by the macrophage backward toward circulation, or TotalD_{B}, resulting in a deficient total backward distance relative to the total forward distance, or TotalD_{B}<TotalD_{F}, and an increase in the number of macrophages trapped in the intima. [symbol]
[N-boxᵥ]_{MΦ}→↓[p300·GABP·N-box_{TF}] AND ↓[TNC_{mRNA}]→↑[TF_{mRNA}]→↑Adhesion curve→↓Skewness of V_{B} curve→↓TotalD_{B}→↑[MΦ in minima] such that TotalD_{F}>TotalD_{B}→↑[MΦ trapped in intima]
Sequence of quantative events 45: Predicted effect of foreign N-boxes on number of trapped macrophages.

Note: TotalD_{B} decreases twice, once, as a response to the coordination-induced decrease in TotalD_{F}, and a second time, as a response to the microcompetition-induced increase in TF transcription. The prediction is also illustrated in FIG. 91.

Microcompetition with the viral N-boxes moves the macrophages from point Mφ₀ to Mφ₁, indicating a shorter backward distance, and an increase in the number of macrophages trapped in the intima.

(b) Histological Observations

Consider the following two photomicrographs of atherosclerotic plaque (Stary 1995⁶¹⁷, FIGS. 1 and 2).

The photomicrographs show a layer of connective tissue covering a lipid core. The core consists of ECM bound oxLDL. The connective tissue consists of smooth muscle cells and a variable number of macrophages. This type of atheroma is called a fibrous cap (Virmani 2000⁶¹⁸). The following table presents some observations typical of fibrous caps and their explanation according to the trucking model of LDL clearance.

TABLE 13
Some observations typical of fibrous caps and their explanation
according to the trucking model of LDL clearance.

| | Explanation |
|---|---|
| Observation | (Based on the trucking model of LDL |
| (Based on Guyton 1995⁶¹⁹) | clearance) |
| The lipid core is formed | Fatty streaks are trapped macrophage- |
| concurrently | derived foam cells. Since the lipid core |
| with fatty streaks. | consists of the oxLDL not cleared by the |
| | trapped macrophages, the lipid core should |
| | formed concurrently with fatty streaks |
| The lipid core has a | Since the macrophage-derived foam cells are |
| tendency to extend | trapped in a superficial depth, the lipid core |
| from a position initially | should have a tendency to extend from a |
| deep in the intima | position initially deep in the intima toward |
| toward the lumen of | the lumen of the artery with increasing age. |
| the artery with | |
| increasing age. | |
| The lipids in the core | The source of lipid in the intima is pollution |
| region seem to | of plasma lipid. The core is a result of failed |
| originate directly from | clearance of these lipids; therefore, the lipids |
| plasma lipoproteins | in the core region should show |
| and not from foam cell | characteristics of plasma lipoproteins. |
| necrosis. | |
| Foam cells are usually | The trapped macrophage-derived foam cells |
| seen in the intima | form a layer between the endothelium and |
| in the region between | the internal elastic lamina. The core is |
| the core and the | formed between the trapped cells and the |
| endothelial surface. | internal elastic lamina. Therefore, the foam |
| | cells should be seen in the intima in the |
| | region between the core and the endothelial |
| | surface. |
| The concentration of | The area near the endothelium is at the tail of |
| foam cells near the | the distribution of the distance macrophage- |
| endothelium is low. | derived foam cells travel back toward |
| | circulation. Therefore, the concentration of |
| | foam cells near the endothelium should be |
| | low. |

(c) Effect on Smooth Muscle Cells Migration

(i) Prediction: Deceased SMC Migration

Infection with a GABP virus intensifies microcompetition for GABP in the infected smooth muscle cell, which increases TF transcription, shifts-up the adhesion curve, and increases the skewness of the TF propelled velocity curve. Consider the following sequence of quantitative events. [symbol]
[N-boxᵥ]_{Medial SMC}→↓[p300·GABP·N-box_{TF}]→↑[TF_{mRNA}]→↑Adhesion curve→↑Skewness of V_{B} curve→↓TotalD_{Toward the intima}→↓[SMC in intima]
Sequence of quantative events 46: Predicted effect of foreign N-boxes on number of SMC in intima.

The increase in number of viral N-boxes increases skewness of the TF propelled backward velocity curve, decreases total distance migrated by SMC toward the intima, and the number of SMC in the intima (see details above).

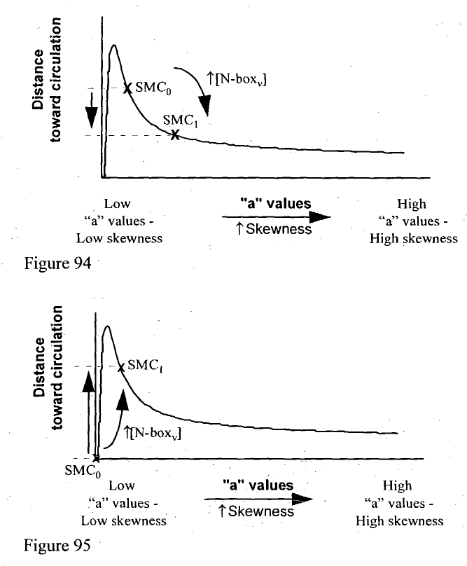

The prediction is also illustrated in FIG. 94.

The N-boxes in the medial and intimal SMC shift the cells from point SMC₀ to SMC₁, indicating a shorter distance toward the intima, and a decrease in the number of SMC in the intima.

Conclusion: An infection of monocytes and smooth muscle cells with a GABP virus transforms an area in the vascular wall polluted with LDL into an atherosclerotic lesion characterized as a thin, unstable, fibrous cap.

(d) Histological Observations

Several studies reported an increase in number of macrophages and a decrease in number of smooth muscle cells in thin, unstable, fibrous caps (Loukas 2002⁶²⁰, Bauriedel 1999⁶²¹, Dangas 1998, ibid). See also a recent review on formation of fibrous cap (Newby 1999⁶²²).

(3) Excessive Skewness and Intimal Thickening

(a) Macrophages

Consider an area in the intima clear of LDL pollution. What is the predicted effect of the clear intima on macrophage migration?

(i) Prediction: No Mφ Migration

The clear intima does not attract monocytes.

(b) Smooth Muscle Cells

Consider an area in the intima populated with SMC latently infected with a GABP virus. What is the predicted effect of the infection on SMC migration?

(i) Prediction: Increased SMC Migration

Infection with a GABP virus intensifies microcompetition for GABP, which increases TF transcription, shifts-up the adhesion curve, and increases the skewness of the TF propelled velocity curve. Consider the following sequence of quantitative events. [symbol]
[N-boxᵥ]_{Medial SMC}→↓[p300·GABP·N-box_{TF}]→↑[TF_{mRNA}]→↑Adhesion curve→↑Skewness of V_{B} curve→↑TotalD_{Toward the intima}→↑[SMC in intima]
Sequence of quantative events 47: Predicted effect of foreign N-boxes in medical SMC on number of SMC in intima assuming no LDL pollution.

The viral N-boxes increase skewness of the TF propelled backward velocity curve, increase distance traveled toward the intima, and increase the number of SMC in the intima (see details above).

The prediction is also illustrated in FIG. 95.

Microcompetition with the viral N-boxes shifts the SMC from point SMC₀ to SMC₁, indicating a longer distance toward the intima, and an increase in the number of smooth muscle cells in the intima. Note that when SMC₁ is positioned on the increasing side of the curve, the result is similar.

(c) Histological Observations

An increase in the number of smooth muscle cells in the intima with no increase in the number of macrophages is a common observation in diffuse intimal thickening (Nakashima 2002⁶²³). On the difference between eccentric and diffuse intima thickening see Stary 1992⁶²⁴.

(4) Other GABP Regulated Genes

Rb, Fas, and p-selectin are also GABP regulated genes (for Rb and Fas, see chapter on cancer, p 358, for p-selectin see Pan 1998⁶²⁵). Microcompetition with a GABP virus can, therefore, also modify trucking cell recruitment, cell proliferation, and cell apoptosis.

(5) Viruses in Atherosclerosis

The idea of infection as a risk factor for atherosclerosis and related cardiovascular diseases is more than 100 years old. However, it was not until the 1970s that experimental data was published supporting the role of viruses in atherosclerosis. The mounting evidence linking infectious agents and atherosclerosis prompted the scientific community to organize the International Symposium of Infection and Atherosclerosis, held in Annecy, France, Dec. 6-9, 1998. The main objective of the symposium was to evaluate the role of infection in the induction/promotion of atherosclerosis on the basis of evidence from recent data on pathogenesis, epidemiologic and experimental studies and to define prevention strategies and promote further research. Consider the following studies presented at the symposium. The studies were published in a special issue of the American Heart Journal (see American Heart Journal, November 1999).

Chiu presented a study that found positive immunostaining for C pneumoniae (63.6%), cytomegalovirus (CMV) (42%), herpes simplex virus-1 (HSV-1) (9%), P gingivalis (42%), and S sanguis (12%) in carotid plaques. The study found 1 to 4 organisms in the same specimen (30%, 24%, 21%, and 6%, respectively). The microorganisms were immunolocalized mostly in macrophages (Chiu 1999⁶²⁶).

In a critical review of the epidemiological evidence, Nieto suggested: “most epidemiologic studies to date (Nieto 1999⁶²⁷, table I and II) have used serum antibodies as surrogate of chromic viral infection. However, there is evidence suggesting that serum antibodies may not be a valid or reliable indicator of chromic or latent infections by certain viruses. In a pathology study of patients undergoing vascular surgery for atherosclerosis serology, for example, for the presence of serum cytomegalovirus antibodies was not related to the presence of cytomegalovirus DNA in atheroma specimens.” However, according to Nieto, four studies, Adam 1987⁶²⁸, Li 1996⁶²⁹, Liuzzo 1997⁶³⁰, and Blum 1998⁶³¹ showed strong positive associations between CMV and clinical atherosclerosis. A strong association was also found in a 1974 survey of the participants in the Atherosclerosis Risk in Communities (ARIC) study between levels of cytomegalovirus antibodies and the presence of sub-clinical atherosclerosis, namely carotid intimal-medial thickness measured by B-mode ultrasound (Nieto 1999, ibid).

Nieto also reported results of a prospective study of clinical incident coronary heart disease (CHD). The study was a nested case-control study from the Cardiovascular Health Study (CHS) conducted in an elderly cohort. Preliminary results from this study found no association between cytomegalovirus antibodies at baseline and incident CHD over a 5-year period. However, HSV-1 was strongly associated with incident CHD, particularly among smokers (odds ratio [OR] 4.2). It should be noted that a more recent prospective study of CMV, HSV-1 in CHD found that participants in the Atherosclerosis Risk in Communities Study (ARIC) study with highest CMV antibody levels at base line (approximately upper 20%) showed increased relative risk (RR, 1.76, 95% confidence interval, 1.00-3.11) of CHD incidents over a 5-year period, adjusted for age, sex and race. After adjustment for the additional covariates of hypertension, diabetes, years of education, cigarette smoking, low-density lipoprotein and high-density lipoprotein cholesterol levels, and fibrinogen level, the RR increased slightly. The study found no association between CHD and the highest HSV-1 antibody levels (adjusted RR, 0.77; 95% confidence interval, 0.36-1.62) (Sorlie 2000⁶³²).

Nieto 1999 (ibid) also mentioned some recent studies, which documented increased risk of restenosis after angioplasty in patients with serologic evidence of cytomegalovirus infection. For instance, Nieto (1999, ibid) reported a study by Zhou and colleagues, which included 75 consecutive patients undergoing directional coronary atherectomy for symptomatic coronary artery disease. Six months after atherectomy, the cytomegalovirus-seropositive patients showed significantly greater decrease in luminal diameter and significantly higher rate of restenosis compared to controls (43% vs. 8% OR 8.7). These results were independent of known cardiovascular disease (CVD) risk factors.

Finally, Nieto mentioned that cytomegalovirus infection has been associated with another form of atherosclerotic disease: accelerated atherosclerosis in the coronaries after heart transplantation. In the first study showing this association, cytomegalovirus serology after transplantation seemed to be one of the most significant predictors of graft atherosclerosis and survival in general. The difference was independent of serologic status before transplantation and presence of symptomatic infection. Subsequent studies reported similar observations.

Based on these studies Nieto concluded: “despite its limitations, the epidemiologic evidence reviewed above is consistent with a broad range of experimental and laboratory evidence linking viral (and other) infections and atherosclerosis disease.”

In a review of animal studies, Fabricant 1999⁶³³ described their experiments with Marek's disease herpesvirus (MDV). The initial experiment used 4 groups of specific pathogen-free (SPF) white leghorn chickens, P-line cockerels of the same hatch, genetically selected for susceptibility to MDV infection. Groups 1 and 2 were inoculated intratracheally at 2 days of age with 100 plaque-forming units of clone-purified, cell free, CU-2 strain of low-virulence MDV. Groups 3 and 4 were controls. For the first 15 weeks, all birds in the 4 groups were fed the same commercial low-cholesterol diet (LCD). Beginning with the 16th and ending with the 30th week, MDV-infected group 2 and uninfected group 4 were placed on a high-cholesterol diet (HCD). The other two groups remained on LCD. Atherosclerotic lesions visible at gross inspection were only observed in MDV-infected birds of groups 1 (LCD) and 2 (HCD). These arterial lesions were found in coronary arteries, aortas, and major arterial branches. In some instances, the marked atherosclerotic changes involved entire segments of the major arteries practically occluding the arterial lumen. Other arterial lesions visible at gross inspection were observed as discrete plaques of 1 to 2 mm. These arterial lesions were not found in any of the uninfected birds of group 3 (LCD) or the uninfected hypercholesterolemic birds of group 4. Many proliferative arterial lesions with intimal and medial foam cells, cholesterol clefts, and extracellular lipid and calcium deposits had marked resemblance to chronic human atherosclerotic lesions. Moreover, immunization prevented the MDV-induced atherosclerotic lesions.

The main conclusion of the symposium was that “although studies are accumulating that indicate a possible relation between infection and atherosclerosis, none of them has yet provided definite evidence of a causal relation. . . . Moreover, the demonstration of a causative role of infectious agents in atherosclerosis would have an enormous impact on public health” (Dodet 1999⁶³⁴) (A similar view is expressed in a review published recently, see Fong 2000⁶³⁵).

What is “definitive evidence?” What evidence will convince Dodet, and others, that viruses are not merely associated with atherosclerosis but actually cause the disease?

The research on viruses in cancer provides an answer. According to zur Hausen 1999-II⁶³⁶: “The mere presence of viral DNA within a human tumor represents a hint but clearly not proof for an aetiological relation. The same accounts for seroepidemiological studies revealing elevated antibody titres against the respective infection.” What constitutes a proof is evidence that meets the following four criteria, specifically the fourth one. According to zur Hausen, “the fourth point could be taken as the most stringent criterion to pinpoint a causal role of an infection.”

The fourth point requires uncovering the sequence of events that leads from viral infection to cell transformation, or an understanding of the mechanism that relates a viral infection and cancer. Crawford 1986⁶³⁷ and Butel 2000⁶¹⁸ emphasize the significance of such understanding. According to Crawford (1986, ibid): “one alternative approach to understudying the role of the papillomaviruses in cervical carcinoma is to identify the mechanisms by which this group of viruses may induce the malignant transformation of normal cells.” According to Butel (2000, ibid): “molecular studies detected viral markers in tumors, but the mechanism of HBV involvement in liver carcinogenesis remains the subject of investigation today.” When the other kind of evidence is in place, uncovering the sequence of events, or an understanding of the mechanism, turns a mere association into a causal relation.

TABLE 14
zur Hausen criteria for defining a causal
role for an infection in cancer.

1. Epidemiological plausibility and evidence that a virus infection
represents a risk factor for the development of a specific tumor.
2. Regular presence and persistence of the nucleic acid of the
respective agent in cells of the specific tumor.
3. Stimulation of cell proliferation upon transfection of the
respective genome or parts therefrom in corresponding tissue
culture cells.
4. Demonstration that the induction of proliferation and the
malignant phenotype of specific tumor cells depends on functions
exerted by the persisting nucleic acid of the respective agent.

The discovery of microcompetition and its effect on trucking cells and SMC migration provides the sequence of events that leads from an infection with a GABP virus and atherosclerosis, or the mechanism, that related such infection with atherosclerosis. This discovery seems to supply the missing “definitive evidence” (Dodet 1999, ibid, see above) that turns the proposed association between viruses and atherosclerosis into a causal relation.

(6) Appendix

(a) TF Gene

Tissue factor (TF) is a GABP suppressed gene. Consider the following observations.

(i) Transcription Related Observations

(a) ETS and (−363, −343), (−191, −172)

A study (Donovan-Peluso 1994⁶³⁹) used DNase I footprinting to map the sites of protein-DNA interaction on the (−383, 8) fragment of the TF promoter. The study used nuclear extracts prepared from uninduced and lipopolysaccharide-induced THP-1 monocytic cells. Six regions were identified. Region number 7 (−363, −343) and region number 2 (−191, −172) contain an N-box. THP-1 extracts formed two complexes on a consensus N-box. Both complexes were competed with excess unlabeled N-boxes and 200-fold excess of a (−363, −343) probe. The (−191, −172) probe, although not as effective as the (−363, −343) probe, showed approximately 30% decrease in formation of the N-box complex (Donovan-Peluso 1994, ibid, FIG. 9).

Another study (Groupp 1996⁶⁴⁰) used the (−231, −145) fragment of the TF promoter as probe. Nuclear extracts prepared from uninduced and lipopolysaccharide-induced THP-1 monocytic cells formed two complexes on the (−231, −145) probe. To characterize the proteins that interact with the DNA sequence, the study used the sc-112× antibody from Santa Cruz Biotechnology. According to the manufacturer literature, the antibody has broad cross-reactivity with members of the ETS family. Incubation of the antibody with the nuclear extracts abrogated formation of the upper complex on the (−231, −145) probe (Groupp 1996, ibid, FIG. 5).

Note that the sc-112× antibody was used in studies with sites known to bind GABP, for example, the HER2/neu, bcl-2, and interleukin 12 promoters. Hence, it is possible that the transcription factor that binds the TF promoter Groupp 1996 (ibid) is GABP.

(b) (−363, −343) Factor and TF Transcription

Holzmuller 1999⁶⁴¹ calls the (−363, −343) fragment of the TF promoter the Py-box. A deletion of the 5′-half of the Py-box increased expression of a luciferase reporter gene (Holzmuller, 1999, ibid, FIGS. 3A and B). The relative increase was similar for LPS induced and non-treated cells and was independent of the existence of the NF-κB site (Holzmuller 1999, ibid, FIG. 3C). Mutation of the N-box part of the Py-box resulted in complete loss of binding activity to the Py-box.

Note: Another study (Fan 1995, ibid) showed an increase in TF transcription after truncation of the (−383, −278) fragment of the TF promoter (Fan 1995, ibid, FIG. 5). Such increase also indicates the existence of a suppresser in this fragment.

(c) (−191, −172) and NF-κB

A study (Hall 1999⁶⁴²) stimulated THP-1 monocytic cells with LPS for various times, up to 24 h. The results showed increased TF mRNA by 30 min, peak at 1 h, considerable drop by 2 h, and return to pre-induction levels at subsequent times (Hall 1999, ibid, FIG. 1). The study also conducted EMSA using the (−213, −172) fragment of the TF promoter. The results showed appearance of two complexes, marked III and IV, at 30 min, peak binding at 1-2 h, and disappearance at 4 h. A 100-fold molar excess of (−213, −172) as probe, or a NF-κB consensus oligonucleotide, competed with the original TF fragment for the two complexes (Hall 1999, ibid, FIG. 2B). Treatment with an anti-p64, and to a lesser extent, an anti-c-Rel antibody, resulted in a supershift of complex III.

The study also provided evidence indicating LPS-mediated proteolysis of IκB and translocation of p65 and c-Rel from the cytoplasm to the nucleus. Western blot analyses showed limited availability of p65 in the nucleus of unstimulated cells. LPS induction resulted in nuclear appearance of p65 after 10 minutes, peak at 1 h, and decline by 2 h. A concomitant decrease in cytoplasmic p65 corresponded to the observed increase in nuclear p65 (Hall 1999, ibid, FIG. 4).

These observations indicate binding of NF-κB to the (−213, −172) fragment of the TF promoter.

Note: The study also showed lower affinity of the NF-κB complex to the NF-κB site compared to the affinity of the complex on the adjacent proximal AP1 site.

(d) Competition for(−191, −172)

Donovan-Peluso 1994 (ibid, see above) showed that the (−191, −172) probe was less effective in competing with the consensus N-box compared to the (−363, −343) probe. According to the authors, the data suggest that there might be competition for binding to the (−191, −172) fragment between NF-κB and an ETS related factor. In such case, NF-κB binding to a (−191, −172) probe decreases the concentration of the probe available for ETS binding. The competition can explain the decreased ability of (−191, −172) to compete for ETS binding relative to (−363, −343). Moreover, the NF-κB site and the N-box in the (−191, −172) fragment overlap. The presence of overlapping sites also suggests competition where occupancy by either factor might preclude binding by the other.

(e) Conclusion: GABP Virus and TF Transcription

Microcompetition between a GABP virus and the TF promoter decreases availability of the ETS related factor for binding with the TF promoter. NF-κB binding to (−191, −172) increases transcription. Competition between NF-κB and the ETS related factor for (−191, −172) suggests that the decrease in availability of the ETS related factor in the nucleus increases binding of NF-κB to the (−191, −172) fragment and increases TF expression. In terms of transefficiency, TransE(ETS related factor)<0 and TransE(NF-κB)>0. Therefore, a decrease in binding of the ETS related factor to the TF promoter stimulates the positive effect of NF-κB on TF transcription (see chapter on transefficiency, p 140).

In addition, binding of the ETS related factor to the (−363, −343) fragment suppresses transcription. Suppression is similar in extracts from untreated, LPS-, and TNFα-induced cells. Moreover, suppression is independent of NF-κB binding. The observation suggests that the ETS related factor suppress transcription in quiescent cells and maintains a moderate level of transcription in activated cells (Holzmuller 1999, ibid). The decrease in availability of the ETS related factor decreases the (−363, −343)-mediated suppression and increases TF expression.

The GABP virus microcompetes with the TF promoter for the ETS related factor, and therefore, increases TF expression.

(ii) Transfection Related Observations

(a) Observations

A few studies measured expression of TF relative to an internal control. The studies used two controls CMVβgal (Moll 1995⁶⁴³, Nathwani 1994⁶⁴⁴ and PRSVCAT (Mackman 1990⁶⁴⁵). Although the studies used different transfection protocols, Moll 1995 (ibid) used psoralen-, and UV-inactivated biotinylated adenovirus and streptavidine-poly-L-lysine as vectors for DNA delivery, Nathwani 1994 (ibid) used electroporation, and Mackman 1990 (ibid) used DEAT-dextran, all studies reported an increase in TF expression relative to a promoterless plasmid. According to Moll, et al., (1995, ibid): the cells “are being already partially activated following the transfection procedure.” The level of activation was similar in unstimulated and LPS stimulated cells.

Note: TF on the cell surface can be deactivated through encryption (Nemerson 1998⁶⁴⁶, Bach 1997⁶⁴⁷), Therefore, when measuring the effect of an exogenous event on TF the possible difference between TF concentration and activity should be considered.

(b) Conclusion: GABP and TF Transcription

The internal controls include promoters of GABP viruses, which decrease availability of GABP to the TF promoter. The control plasmids increase TF expression. Therefore, GABP is a suppresser of TF transcription.

6. Stroke

a) Introduction

Stroke (cerebrovascular accident, CVA) is cardiovascular disease resulting from disrupted blood flow to the brain due to occlusion of a blood vessel (ischemic stroke) or rupture of a blood vessel (hemorrhagic stroke). Interruption in blood flow deprives the brain of oxygen and nutrients, resulting in cell injury in the affected vascular area of the brain. Cell injury leads to impaired or lost function of body parts controlled by the injured cells. Such impairment is usually manifested as paralysis, speech and sensory problems, memory and reasoning deficits, coma, and possibly death.

Two types of ischemic strokes, cerebral thrombosis, and cerebral embolism, account for about 70-80 percent of all strokes. Cerebral thrombosis, the most common type of stroke, occurs when a blood clot (thrombus) forms, blocking blood flow in an artery supplying blood to the brain. Cerebral embolism occurs when a wandering clot (an embolus), or another particle, forms in a blood vessel away from the brain, usually in the heart. The bloodstream carries the clot until it lodges in an artery supplying blood to the brain blocking the flow of blood.

b) Microcompetition with Foreign DNA

Microcompetition with foreign DNA causes atherosclerosis. Like coronary artery occlusion, atherosclerosis in arteries leading blood to the brain (such as carotid artery), or in the brain, may result in arterial occlusion through plaque formation or plaque rupture, and in situ formation of a thrombus (see chapter on atherosclerosis, p 161). Lammie 1999⁶⁴⁸ reports observations indicating similar pathogenesis in coronary artery disease (CAD) and stroke. In general, numerous studies reported an association between atherosclerosis and stroke (see, for instance, Chambless 2000⁶⁴⁹, O'Leary 1999⁶⁵⁰).

In addition, microcompetition with foreign DNA increases TF expression on circulating monocytes. Monocytes originate from CD34+progenitor cells (Hart 1997⁶⁵¹, FIG. 3), which are permissive for a GABP viral infection (for instance, Zhuravskaya 1997⁶⁵² demonstrated a persistent infection of human cytomegalovirus (HCMV), a GABP virus, in bone marrow (BM) CD34+cells, see also, Maciejewski 1999⁶⁵³, Sindre 1996⁶⁵⁴). Infection of CD34+with a GABP virus increases TF expression on circulating monocytes, which increases the probability of a coagulation event and formation of an embolus. Consistent with such a sequence of quantitative events, several studies reported excessive TF expression in stroke patients (see, for instance, Kappelmayer 1998⁶⁵⁵).

7. Autoimmune Disease

a) Conceptual Building Blocks

(1) Deletion vs. Retention, Th1 vs. Th2

Dendritic cells (DCs) and macrophages are professional antigen presenting cells (professional APCs). For simplicity, let the symbol DCs represent both types of professional APCs.

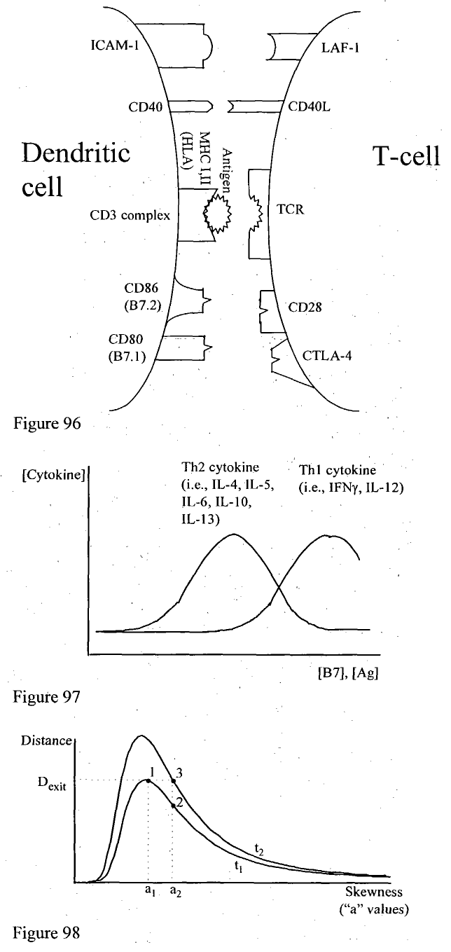

DCs bind T-cells. FIG. 96 illustrates some molecules on the surface of DCs and T-cells that participate in the binding.

The strength of DC and T-cell binding, denoted [DC·T], is a positive function of B7 concentration on surface of a DC, denoted [B7], a negative function of CTLA4Ig concentration on surface of T-cell, denoted [CTLA4Ig], and a positive function of concentration of the major histocompatibility complex (MHC) bound to antigen on a DC, denoted [Ag]. The following function describes these relations.

$$\begin{matrix}[ DC \cdot T ] = & f ( [ B7 ] , & [ CTLA4Ig ] , & [ Ag ] ) \\ & ( + ) & ( - ) & ( + )\end{matrix} \qquad Function 31$$

A positive sign under [87] means positive relation, that is, increase in B7 surface concentration increases the strength of DC and T-cell binding. A negative sign under a variable indicates negative relation.

Assume a greater than zero rate of substitution between [B7] and [Ag], that is, increase in [B7] can compensate, to a certain degree, for a decrease in [Ag], and vise versa.

The strength of DC and T-cell binding determines CD8+ retention vs. deletion, and Th1 vs. Th2 differentiation.

(a) CD8+ Retention vs. Deletion

Low [DC·T] increases peripheral CD8+ proliferation and deletion. The deletion is specific for the antigen presented on the MHC. High [DC·T] increases peripheral CD8+ proliferation and retention. T-cells do not differentiate between self or foreign antigens, the cells respond only to [DC·T].

Define antigen specific peripheral tolerance as deletion of T-cells specific for this antigen. Then, a decrease in [DC·T] increases tolerance.

(b) Th1 vs. Th2 Differentiation

T helper lymphocytes can be divided into two subsets of effector cells based on their function and the cytokines they produce. The Th1 subset of CD4+ T-cells secretes cytokines usually associated with inflammation, such as interleukin 2 (IL-2), interleukin 12 (IL-12), interferon γ (IFNγ), and tumor necrosis factor β (TNFβ), and induces cell-mediated immune responses. The Th2 subset produces cytokines such as interleukin 4 (IL-4), interleukin 5 (IL-5), interleukin 6 (IL-6), interleukin 10 (IL-10), and interleukin 13 (IL-13), which help B cells to proliferate and differentiate, and is therefore associated with humoral immune responses (see recent review Constant 1997⁶⁵⁶).

In relevant physiological conditions, low [DC·T] induces CD4+ differentiation into Th2, while high [DC·T] induces Th1 differentiation. According to the function above, an increase in [B7] or [Ag] increases the strength of DC and T-cell binding, or [DC·T]. Therefore, an increase in either [B7] or [Ag] increases the probability of Th1 vs. Th2 differentiation. FIG. 97 illustrates the conclusion.

The observations in Rogers 1999⁶⁵⁷ are consistent with such a relation. Naive CD4 cells were stimulated with varying doses of moth cytochrome c (MCC) presented on splenic APC and cultured for 4 or 12 days. An equivalent number of surviving T-cells was restimulated with a single dose of Ag and assayed for secretion of Th1 and Th2 cytokines. The results showed that the length of differentiation period (4 or 12 days) affects the cytokine profile induced by varying doses of native peptide. Overall, after 12 days of differentiation, lower doses of high affinity peptides produced T-cells mostly secreting Th2 cytokines. In contrast, higher doses of high affinity peptides increased the number of T-cells that secreted Th1 cytokines. The study summarized these and other observations in a figure (Roger, ibid, FIG. 7) that is almost identical to the figure above. The figure for T-cells after 4 days in culture is different. However, since autoimmune disease is a chronic condition, extended exposure to APC more closely approximates the in vivo environment of CD4+ T-cells.

(2) Antigen Internalization and [Ag], [B7]

An antigen is a molecule that induces an internalization response in DCs (e.g. phagocytosis, cell engulfment, etc.). Cell debris, apoptotic cells, foreign proteins, etc. are antigens, that is, they activate an internalization response by DC.

An increase in the concentration of internalized antigens stimulates antigen processing and presentation on DCs surface, or [Ag]. The increase in concentration of internalized antigens also increases [B7], thereby increasing costimulation (see, for instance, Rovere 2000⁶⁵⁸ and Rovere 1998⁶⁵⁹ for observations consistent with this concept).

Consider a stationary DC. An increase in antigen concentration in the DC environment increases the probability of antigen internalization. Consider a DC migrating through an environment with fixed antigen concentration. Slower DC migration increases the probability of antigen internalization. Therefore, both an increase in antigen concentration in the cell environment, and a decrease in average cell velocity increase [Ag] and [B7].

Assume an inverse relation between the concentration of internalized antigens and average cell velocity. The decrease in average velocity amplifies a small increase in antigen concentration in the DC environment into large increases in [Ag] and [B7]. Such amplification increases the sensitivity of DC to its environment.

(3) Homing Signal

A source DC releases chemokines. The chemokines direct activated T-cells and additional DCs to the source. Steering of T-cells and new DCs is most effective when the source DC is stationary, otherwise, T-cells and new DC need to chase a moving target.

Some of the chemokines secreted by DCs are RANTES (regulated upon activation, normal T-cell expressed and secreted), MIP-1α, and MIP-1β (macrophage-inflammatory protein-1α and 1β, respectively). CCR5 is a receptor for these chemokines variably expressed on monocytes, activated T-cells, natural killer cells, and dendritic cells.

(4) Cytotoxic T Lymphocytes (CTL)

Assume a stationary source DC releasing chemokines. Antigen specific CTL enter the tissue near the stationary DC and bind and destroy all target cells, that is, cells which present the specific antigen on their MHC. The target cells include the stationary DC and all tissue cells that present the antigen.

b) Model

Define damaged tissue as tissue that shows abnormal morphology. Define tolerance, activation, and autoimmune disease as an immune reaction that results in no tissue damage, reversible, self-correcting tissue damage, and irreversible tissue damage, respectively. Note that these definitions are different from acute vs. chronic immune activation.

The following sections present a model that describes the conditions that determine the type of the immune reaction.

(1) Tolerance

Tolerance is an immune reaction that results in no tissue damage.

Terminology: In the chapter on atherosclerosis, foam cell migration back into circulation was called backward motility. Since backward motility essentially means out of tissue migration, this chapter uses the same term to describe DC migration from tissue to a lymph vessel.

DCs continuously enter tissues. While in tissue the cells collect, process, and present antigens on MHC. Internalized antigens induce oxidative stress, which decreases binding of GABP to the tissue factor (TF) promoter, increases TF expression (see effect of GABP on TF expression in chapter on atherosclerosis, p 161). TF propels the DC backward motility, or migration out of tissue and into a lymph vessel. Since backward motility takes a relatively short time, the DCs entering the lymph vessel show only a small increase in [B7]. Moreover, under normal conditions, the concentration of antigens in a DC migration path is low. As a result, DCs entering the lymph vessel also show low [Ag]. In the draining lymph node, DCs bind naive T-cells expressing T-cell receptors (TCR) that match the presented antigens. Since [B7] and [Ag] are low, [DC·T] is low (see function above). As a result, the bound T-cells proliferate and die.

Symbolically,
↑[Antigen]_{cell}_{ⱼ}→↑[Ag]_{cell}_{ⱼ}, ↑[B7], ↑OS→↓[p300·GABP·N-box_{TF}]→↑[TF_{mRNA}]→↑TF_{DC} adhesion curve→↑Skewness of V_{DC} curve→↑Distance_{DC}(t)→↓[T-cell_{antigen}]_{lymph node}
Sequence of quantative events 48: Predicted effect of a small increase in antigen production by a certain cell on number of matching T-cell in lymph node.

The symbol [Antigen]_{cell}_{ᵢ} denotes antigen concentration originating from cellᵢ. The symbol [T-cell_{antigen}]_{lymph node} denotes antigen matching T-cell in the lymph node. Note the difference between the symbols [Antigen]_{cell}_{ᵢ} and [Ag], which denote antigen concentration in the environment and in DCs, and antigen concentration on surface MHC, respectively.

(2) Immune Activation

Activation is an immune reaction that results in reversible tissue damage.

Consider a tissue with an increased local production of an antigen. For simplicity, let the antigen originate from a single cell, called the origin. An increase in antigen concentration in the DC environment increases antigen internalization, which increases cellular free radicals, and TF expression (oxidative stress decreases binding of GABP to the TF gene and increases TF transcription, see chapter on atherosclerosis, p 161). Consider the following sequence of quantitative events.
↑↑[Antigen]_{cell}_{ⱼ}→↑↑[Ag]_{cell}_{ⱼ}, ↑↑[B7], and ↑↑OS→↓↓[p300·GABP·N-box_{TF}]→↑↑[TF_{mRNA}]→↑↑TF_{DC} adhesion curve→↑↑Skewness of V_{DC} curve→↓↓Distance_{DC}(t)
Sequence of quantative events 49: Predicted effect of a large increase in antigen production by a certain cell on distance traveled by DCs migrating near the cell.

The sequence describes the dynamics of the immune system during activation. Low, or normal level activation is denoted with one arrow facing up or down (see tolerance above). Denote increased activation with 2 arrows facing up or down: ↑↑ or ↓↓. Note the different effect of skewness on distance. Low skewness (one arrow up) increased migration distance. Increased skewness (two arrows facing up) decreases migration distance (see details in technical note on cell motility).

Note: Let the parameter “a” represent skewness (see chapter on cell motility, p 145). Denote the level of skewness during tolerance and activation with a_{tolerance} and a_{activation}, respectively. Then, a_{tolerance}<a_{activation} (1 vs. 2 arrows in the corresponding sequences of quantitative events).

The increase in TF expression shifts-up the TF adhesion curve, increases skewness of the DC velocity curve, and decreases the distance traveled by the cell at a given time interval (see details in the technical chapter on cell motility, p 145).

What is the effect of the decrease in migration distance on migration time? FIG. 98 presents graphically the relation between skewness, migration distance, and time (see explanation in the technical note on cell motility).

The distance on the y-axis is expressed for a given time interval, that is, time is a controlled variable of the curve in the figure. An increase in the time interval increases migration distance for a given level of skewness (compare points 2 and 3). The figure illustrates two time intervals, t₂ and t₁, where t₂>t₁. Dₑₓᵢₜ denotes the distance between the origin and the nearest lymphatic vessel, or the distance a cell needs to migrate to exit the tissue and enter the nearest lymphatic vessel. Consider a cell migrating in low antigen concentration. Denote the cell skewness with a₁, and assume that a₁ corresponds to Dₑₓᵢₜ (see figure). In simple terms, consider a cell that under normal antigen concentrations barely makes it to the lymphatic vessel at time t₁. What is the effect of an increase in antigen concentration of this cell?

The increase in antigen concentration increases skewness to a₂. The increase in skewness decreases migration distance. Therefore, to exit the tissue the cell needs a longer migration time, t₂ (points 2 and 3 in figure). Overall, the cell shows lower average velocity (Dₑₓᵢₜ/t₂<Dₑₓᵢₜ/t₁).

Note that the decrease in average velocity might further increase antigen internalization and skewness, resulting in an even larger decrease in average velocity.

Antigen concentration near the origin is not uniform. Some regions contain moderate concentrations, other contain high antigen concentrations. DCs migrating through a region with high antigen concentration show higher skewness compared to cells migrating through a region with moderate antigen concentration. What is the effect of the even higher skewness on DC migration?

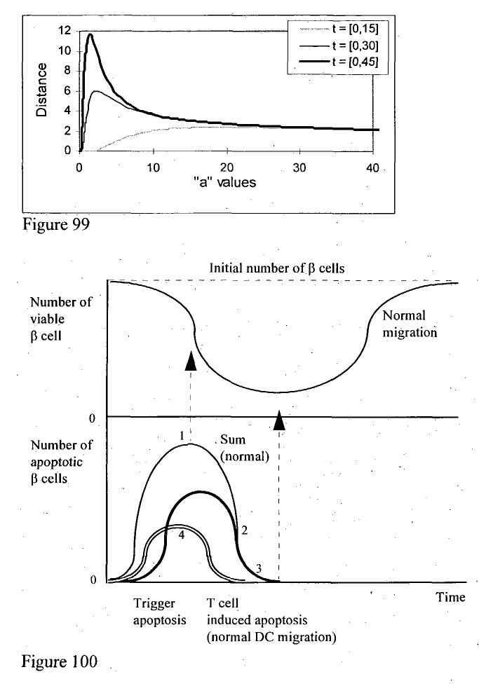

FIG. 99 is a copy of the original figure in the technical note on cell motility.

Note the right tail of the curves. At high enough “a” values, or skewness, an increase in time doe not increase migration distance, the cell is trapped in the tissue. In the figure, high skewness traps the cell at a distance of about 1.5 units from the origin.

An analysis that represents skewness with the “b” instead of the “a” parameter (see chapter on cell motility, p 145) produces similar insights. However, with “b,” the final resting site can be at a zero distance from the origin.

To conclude, some of the cells migrating near the origin, that is, in regions with high antigen concentration, might end up trapped in the tissue near the origin.

Symbolically,
↑↑[Antigen]_{cell}_{ⱼ}→ . . . →↓↓Distance_{Dc}(t)→↑[Trapped DCs]_{origin}
Sequence of quantative events 50: Predicted effect of a large increase in antigen production by a certain cell on number of DCs trapped near the cell.

What is the effect of the increase in the number of trapped cells on T-cell reaction?

Take insulin producing β cells as an example for the tissue in the excessive skewness model of autoimmune disease presented above. Assume β cells increase production of antigens, which increases concentration of antigens in the DC migratory path. The increase might result from injury, infection, transgene expression, etc. (see examples below). Since, in most cases, antigen production involves apoptosis, this initial event will be called “trigger apoptosis.” For simplicity, let trigger apoptosis be self-limiting. The curve illustrating the number of apoptotic β cells over time is bell shaped (see FIG. 100). Assume a fixed level of antigen production per cell. Under such assumption, the curve that represents the number of apoptotic cells can also represent antigen concentration.

DCs continuously migrate through the pancreas. The increase in production of autoantigen increases the concentration of antigens in the cells, which increases skewness of the backward velocity curve. A few DCs reach the lymph vessel, and then th6 draining lymph node, where they present higher [Ag] and [B7] to T-cells, inducing proliferation and retention. Other DCs end up trapped in the tissue near the origin. The trapped cells release chemokines, which home activated T-cells to the site of excessive antigen production. The chemokines also home more DCs to the same site, amplifying the initial reaction. Infiltrating T-cells bind trapped DCs and β cells inducing a second wave of apoptosis, which decreases the number of trapped DCs, production of DCs chemokines, number of infiltrating T-cells and new DCs, and returns the system to tolerance. Since the T-cell induced apoptosis is self-limiting, FIG. 100 presents it with a bell shape curve. Symbolically,
↑↑[Antigen]_{cell}_{ⱼ}→↑↑[Ag]_{cell}_{ⱼ}, ↑↑[B7], and ↑↑OS→↓↓[p300·GABP·N-box_{TF}]→↑↑[TF_{mRNA}]→↑↑TF_{DC} adhesion curve→↑↑Skewness of V_{DC}curve→↓↓Distance_{Dc}(t)→↑[Trapped DCs]_{origin} and ↑[T-cells]_{lymph node}→↑[T-cells]_{cells}→↓[Trapped DCs]_{origin} and ↓[Antigen]_{cell}_{ⱼ}
Sequence of quantative events 51: Predicted effect of a large increase in antigen production by a certain cell on number of DCs trapped near the cell and rate of antigen production.

Note the single arrow next to the antigen concentration at the end of the sequence. Following the decrease in antigen concentration, antigen production returns to normal levels, represented by one arrow facing up in the tolerance sequence above.

Overall, the number of remaining viable 1 cells is equal to the initial number of 1 cells minus the total number of apoptotic cells (that is, initial number of β cells—trigger apoptosis—T-cell induced apoptosis). In FIG. 100, the curve 0,1,2,3 presents the sum of apoptotic cells, and the top half of the figure illustrates the corresponding “number of viable β cells” curve.

Note that the peak of the “sum curve” corresponds to the turn in the S shape of the “number of viable β cells” curve, and the end of the “sum curve” corresponds to the minimum point on the “number of viable β cells” curve (see arrows with doted lines). The right hand side of the “number of viable β cells” curve illustrates β cell neogenesis. The final number of viable β cell is equal the initial number, and therefore, at termination, tissue damage is reversed.

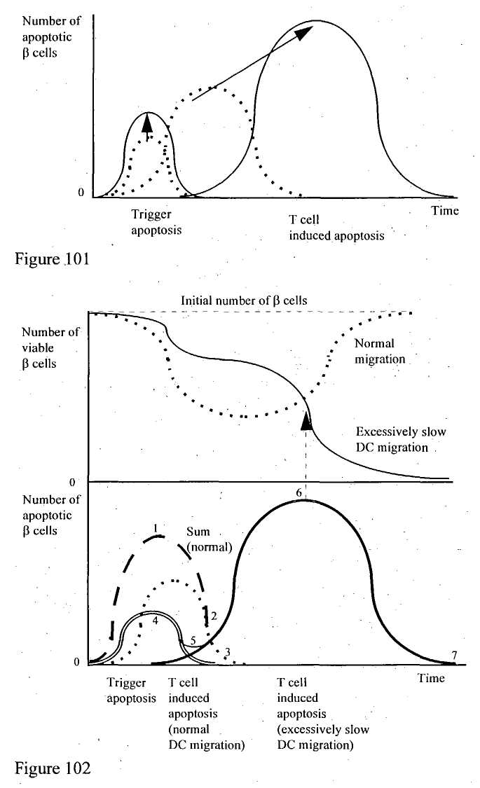

Assume an increase in trigger apoptosis. How do the two peaks respond to such a change? Consider FIG. 101. The increase in trigger apoptosis increases antigen concentration in tissue, and in DCs. The excess oxidative stress increases TF surface expression and skewness, decreases average velocity, and further delays T-cell activation. However, when DCs eventually reach the lymph node, they present higher [Ag] and [B7], and therefore, activate more T-cells (higher probability for activation and retention rather than activation and deletion). In addition, more DCs are trapped in the tissue, which chemoattracts more T-cells, and increases apoptosis. Overall, the increase in trigger apoptosis shifts the second peak to the right and upward (see figure).

Note: In following sections, a shift to the right and upward will be called diagonal increase, and a shift in the opposite direction, diagonal decrease.

(3) Autoimmune Disease

Autoimmune disease is an immune reaction that results in irreversible tissue damage, or abnormal tissue morphology.

A level of skewness a₀, higher than the normal level of skewness during activation, that is, a_{activation}<a₀, will be called “excessively” high. Consider an exogenous event that excessively increases the level of skewness. What is the effect of such event on the immune system?

Notes:

1. Since the event is exogenous, it is independent of antigen concentration in the DCs migration path.

2. The event can be local or systematic.

The following sequence of quantitative events presents the effect of such exogenous events.
↑↑[Antigen]_{cell}_{ⱼ}→↑↑[Ag]_{cell}_{ⱼ}, ↑↑[B7], and ↑↑OS→↓↓[p300·GABP·N-box_{TF}]→↑↑[TF_{mRNA}]→↑↑TF_{DC} adhesion curve→↑↑ [symbol] Skewness of V_{DC} curve→↓↓↓Distance_{Dc}(t)→↑↑[Trapped DCs]_{origin} and ↑↑[T-cells]_{lymph node}→↑↑[T-cells]_{origin}→↑[Cellⱼ-type autoimmune disease]
Sequence of quantative events 52: Predicted effect of an increase in skewness on susceptibility to autoimmune disease.

The boxed arrow next to “Skewness of V_{DC} curve” denotes the exogenous event. Note the addition of one arrow in all subsequent events. The excessive increase in number of T-cells near the origin, and T-cell induced apoptosis, results in permanent cellᵢ-type tissue damage, or cellᵢ-type autoimmune disease (type I diabetes is a β cell-type autoimmune disease).

FIG. 102 presents the effect of excessive skewness graphically.

The exogenous event diagonally increases the second peak. The sum of β cell apoptosis is represented by the two peak curve (0,4,5,6,7). What is the shape of the corresponding “number of viable β cells” curve? Excessive β cell apoptosis induces excessive tissue damage. If the tissue regeneration capacity is limited, there exists a level of β cell apoptosis, which permanently decreases the number of viable β cells. Note that the corresponding “number of viable β cells” curve shows complete destruction of β cells. Under conditions of limited regeneration capacity, the damage is irreversible, and therefore, describes autoimmune disease.

c) Predictions and Observations

The following sections compare the predicted and observed effects of different exogenous events on immune system dynamics.

(1) Animal Models

(a) Tolerance

A recent review summarizes many observations related to issues of ignorance and tolerance (Heath 1998⁶⁶⁰). Based on these observations, Heath, et al., (1998, ibid) concluded: “taken together, there is compelling evidence that in order to maintain self-tolerance a specialized APC is capable of capturing tissue antigens, transporting them to the lymphoid compartment, i.e., the draining lymph nodes, and presenting them to both naive CD4+ and CD8+ T-cells. . . . This APC appears to be capable of processing exogenous antigens into class I and class II pathways. The above data argue for the existence of a “professional” APC that constitutively induces tolerance to antigens expressed in extralymphoid tissues . . . In studies using transgenic mice expressing different levels of ovalbumin (OVA) in the pancreas, we have recently found that antigen concentration is critical in determining whether such antigens are cross-presented in the draining lymph nodes. . . . The level of antigen expression appears to determine whether an antigen induces cross-tolerance or is ignored by naive T-cells. . . . It is interesting to note that deletion of both CD4+ and CD8+ T-cells is preceded by a period of proliferation, suggesting that the APC responsible for tolerance induction must be capable of activating T-cells into proliferative cycles. Moreover, the APC is a cell capable of trafficking from peripheral tissues to a draining lymph node. Even more importantly for CD8+ T-cell tolerance, this APC must be capable of capturing exogenous antigens and cross-presenting them in a class I pathway. Various cells types have been shown to have the capacity to cross present exogenous antigens in vitro, including myeloid-derived DCs, macrophages, and B cells.”

Unlike the factors regulating the balance between tolerance and ignorance, the factors determining the choice between tolerance and priming are not well understood. According to Heath, et al., (1998, ibid), what determines the choice between tolerance and priming “is probably one of the outstanding questions at the moment.” According to Sallusto 1999⁶⁶¹ in another recent review: “finding the factors that regulate the balance between tolerance and response is now considered the holy grail of immunology.”

(b) Immune Activation

(i) O'Brien 1996

A study (O'Brien 1996⁶⁶²) injected 5-6 week old male C57B1/6 mice with a low-dose (40 mg/kg body weight) of streptozotocin (STZ) per day for five consecutive days. STZ injection induces P cell apoptosis. Consider the following sequence of quantitative events. [symbol]
[STZ]→↑↑↑[β cell apoptosis]_{Trigger}→↑↑↑[Antigen]_{β cell}→↑↑↑OS→↓[p300·GABP·N-box_{TF}]→↑↑↑[TF_{mRNA}]→↑↑↑TF_{DC} adhesion curve→↑↑↑Skewness of V_{DC} curve→↓↓↓Distance_{DC}→↑↑[Trapped DCs]_{origin} and ↑↑[T-cells]_{lymph node}→↑↑[T-cells]_{origin}→↑↑[β cell apoptosis]_{T-cell}→↑[β cell-type autoimmune disease/diabetes]
Sequence of quantative events 53: Predicted effect of streptozotocin on susceptibility to diabetes.

The symbol [β cell apoptosis]_{Trigger} denotes rate of β cell trigger apoptosis (first peak). The symbol [Antigen]_{β cell} denotes β cell antigen concentration both in the environment and in DCs. The symbol [Trapped DCs]_{origin} denotes the number of trapped DCs near the origin. The symbol [T-cells]_{origin} denotes number of T-cells near the origin, called insulitis. The symbol [β cell apoptosis]_{T-cell} denotes the rate of β cell apoptosis induced by the infiltrating T-cells (second peak).

According to the two peak model, the injection should induce a two peak immune reaction that results in diabetes.

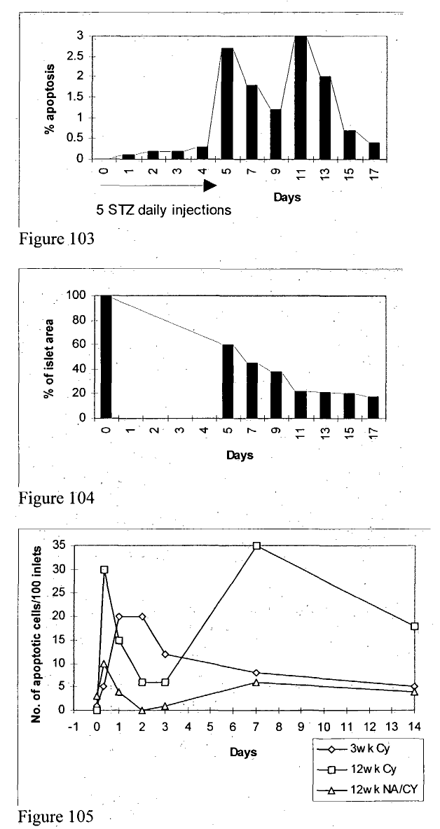

As expected, the observations showed two peaks in β cell apoptosis. The first peak, which was associated with an increase in blood glucose concentration, occurred at day 5. The second peak occurred at day 11, when the islets showed maxim lymphocytic infiltration, or insulitis. FIG. 103 and FIG. 104 summarize the observations (O'Brien 1996, ibid, FIGS. 3, 4).

Insulitis did not begin until day 9, by which time treated animals showed overt diabetes. β-cell apoptosis preceded the appearance of T-cells in the islets and continued throughout the period of insulitis.

The observations in O'Brien 1996 (ibid) are consistent with the predicted effect of streptozotocin injection on the dynamics of immune activation.

(ii) O'Brien 2000

Pancreatic islets are especially susceptible to oxidative stress. A study (Lenzen 1996⁶⁶³) showed low gene expression of the antioxidant enzymes superoxide dismutase (SOD), catalase, and glutathione peroxidase in pancreatic islets compared with various other mouse tissues. Another study (Tiedge 1997⁶⁶⁴) showed that induction of cellular stress by high glucose, high oxygen, and heat shock treatment did not affect antioxidant enzyme expression in rat pancreatic islets or in RINm5F insulin-producing cells. Based on these observations, Tiedge, et al., (1997, ibid) concluded: “insulin-producing cells cannot adapt the low antioxidant enzyme activity levels to typical situations of cellular stress by an upregulation of gene expression.”

Young mice (0-3-weeks old) showed a marked increase in protection against oxidative stress, through changes in glutathione peroxidase/reductase, catalase and superoxide dismutase activities in liver, lung, and kidney tissues, relative to older mice (Harman 1990⁶⁶⁵). Assume DCs in young mice show similar increased protection against oxidative stress, and β cell show a lower level of protection (reasonable assumption in light of Tiedge 1997 (ibid), see above).

A study (O'Brien 2000⁶⁶⁶) administered a single intraperitoneal injection of cyclophosphamide (CY, 150 mg/kg body weight) to 3-, and 12-week old (older) male non-obese diabetic (NOD/Lt) mice. The study also administered, to another group of 12-week old mice, a single intraperitoneal injection of nicotinamide (NA, 500 mg/kg body weight) followed 15 minutes later by a single CY injection.

Prediction 1: According to the two peak model, treatment with CY should induce trigger apoptosis with a smaller diagonal increase in second peak in young compared to older mice.

Prediction 2: If the trigger apoptosis itself is also smaller in 3-week old mice, treatment with an oxidant could possible show a single peak for the sum of β cell apoptosis (see figure above where the T-cell apoptosis partially overlaps the trigger apoptosis).

Prediction 3: Older mice pretreated with antioxidant should show an attenuated two peak response.

Figure presents the observations (O'Brien 2000, ibid, FIG. 3).

Apoptotic β cells were observed within the islets of Langerhans in haematoxylin and eosin-stained sections of the pancreata in all three groups harvested from 8 h until 14 days following treatment. However, the shape of the three curves representing the sum of β cell apoptosis was different. 3-week old mice under CY treatment showed a single peak, 12-week mice under CY showed a two peak curve, and 12-week mice under NA/CY showed an attenuated two peak curve.

The observations in O'Brien 2000 (ibid) are consistent with the predicted effects of CY and NA/CY treatments on the dynamics of immune activation.

(iii) Hotta 1998

A study (Hotta 1998⁶⁶⁷) generated transgenic NOD mice (Tg) that over express the redox-active protein thioredoxin (TRX) in β cells, and compared insulitis and onset of diabetes in transgenic mice and control.

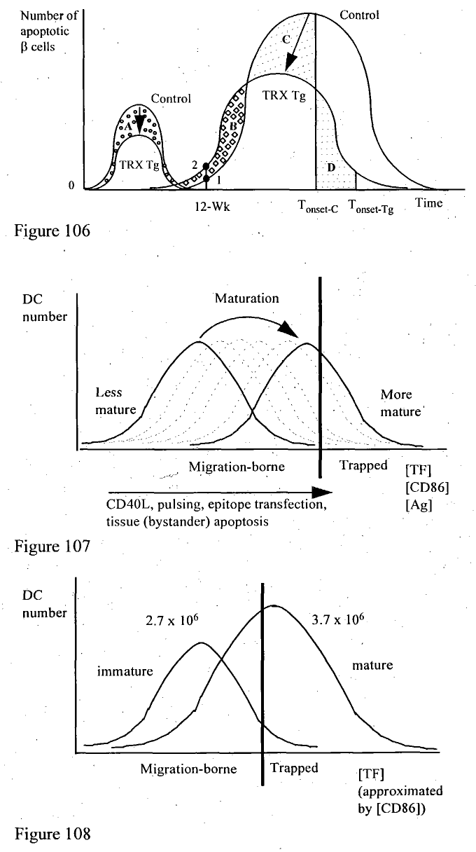

The increased TRX-mediated protection against oxidative stress decreases trigger apoptosis, which decreases the first peak and diagonally decreases the second peak. Consider FIG. 106.

For simplicity, assume that overt diabetes is associates with destruction of a certain, fixed number of β cells (in reality, it is actually a range and not a fixed number, however, the average number of β cells can represent the fixed number in the following analysis). The fixed number is represented by the sum of the areas (integral) under the two peaks. Let T_{onset-Tg} and T_{onset-C} denote the time of diabetes onset in TRX transgenic mice and control, respectively. The two peaks are smaller for the TRX transgenic mice. Therefore, the onset of diabetes in transgenic animals is delayed (T_{onset-Tg}>T_{onset-C}). Consider the areas under the two peaks for the two time intervals [0,T_{onset-Tg}] and [0,T_{onset-C}], that is, the areas from start to onset of diabetes. Since diabetes is associated with a loss of the same number of β cells in both transgenic and control animals, the areas are equal in size. A, B, C, and D represent the changes between the two types of mice. Since the two areas for the time intervals [0,T_{onset-Tg}] and [0,T_{onset-C}] are equal in size, A+C=B+D. Note that a decrease in the size of area B increases the size of area D, or increases the delay in onset of diabetes. Let the distance between points 1 and 2 indicate the size of area B. A small distance between the points indicates a small area B, and therefore, a substantial delay in the onset of diabetes.

According to Hotta 1998 (ibid), average insulitis scores were 1.63±0.32 and 1.57±0.26 in 12-wk-old transgenic and control mice, respectively. Although the difference is not significant statistically, the score for transgenic mice is a little higher compared to control, as predicted by the two peak model. Moreover, the small difference indicates a small area B, and therefore, large area D, or substantial delay in onset of diabetes. As expected, transgenic mice showed a “substantial” delay in onset of diabetes compared to control (week 23 vs. week 14 in transgenic and control mice, respectively). Moreover, at week 32, transgenic mice still showed a marked decrease in cumulative incidence of diabetes compared to control (Hotta 1998, ibid, FIG. 4).

Notes:

1. The “substantial” delay should be interpreted with caution. The study provides only one observation relating the distance between points 1 and 2 (see figure) and the magnitude of the delay. To turn a “substantial” delay to a “statistically significant increase” in the delay, more observations are needed.

2. Similar observations are reported in Kubisch 1997⁶⁶⁸.

Several other studies showed decreased insulitis and delayed diabetes in NOD mice following treatment with antioxidants such as nicotinamide (vitamin B3) (Kim 1997⁶⁶⁹, Reddy 1990⁶⁷⁰), vitamin E (Beales 1994⁶⁷¹), lipoic acid (Faust 1994⁶⁷²), and U78518F (Rabinovitch 1993⁶⁷³).

The observations in these studies are consistent with the predicted effect of antioxidants on the dynamics of immune activation.

(c) Autoimmune Disease

(i) Studies with LCMV

(a) Conceptual Building Blocks

(i) GABP Virus

The following observations suggest that the lymphocytic choriomeningitis virus (LCMV) is a GABP virus. The glycoprotein (GP) promoter of LCMV has two N-boxes at positions (−44, −38) and (−3, +3). The distance between the two N-boxes is 35 bp. Of the dozens of known ETS factors, only GABP, as a tetrameric complex, binds two N-boxes. Typically, the N-boxes are separated by multiples of 0.5 helical turns (HT) (see discussion and references in chapter on obesity, the section on the hormone sensitive lipase (HSL) gene, p 314). There are 10 bp per HT. The 35 bp; or 3.5 helical turns separating the N-boxes in the GP promoter, are consistent with GABP heterotetramer binding.

(ii) Persistent Infection in DCs

The following observations suggest that the LCMV ARM 53b strain establishes persistent infections in DCs. LCMV strains can be divided into two groups. The first group marked CTL-P+, includes viruses isolated from lymphocytes or macrophages obtained from CD4, perforin, and TNFα knock out mice, infected for at least 7 months. The viruses failed to generate LCMV-specific CTL responses, and showed that characteristics of a persistent infection. The second group marked CTL+P−, includes viruses isolated from the CNS of TNFα knockout mice. The viruses showed a potent LCMV-specific CTL response, which cleared the virus within 2 weeks and left no evidence of persistent infection. The Armstrong (ARM) 53b strain is a CTL-P+virus (Sevilla 2000⁶⁷⁴, Table T). According to Sevilla, et al., (2000, ibid): “first, DCs are the primary cell infected in vivo by CTL-P+LCMV variants; second, CTL-P+viruses astoundingly infect >50% of CD11c+(a cellular marker for most DCs in mouse lymphoid tissue) and DEC-205+(antigen expressed on DCs in lymphoid tissues) cells.”

(b) Diabetes

(i) RIP-GP, RIP-NP Transgene

RIP-GP and RIP-NP transgenic mice express the viral glycoprotein (GP) or nucleoprotein (NP) from lymphocytic choriomeningitis virus (LCMV) under control of the rat insulin promoter in pancreatic β cells. Assume that some of the RIP-GP and RIP-NP transgenic mice are latently infected with a GABP virus. The mice should show diabetes. Consider the following sequence of quantitative events.
↑ [symbol] [Antigen]_{β cell}→↑↑[Ag]_{β cell}, ↑↑[B7], and ↑↑OS→↓↓ [symbol] [p300·GABP·N-box_{TF}]→↑↑↑[TF_{mRNA}]→↑↑↑TF_{Dc} adhesion curve→↑↑↑Skeweness of V_{DC} curve→↓↓↓Distance_{DC}(t)→↑↑[Trapped DCs]_{origin} and ↑↑[T-cells]_{lymph node}→↑↑[T-cells]_{origin}→↑[Cellᵢ-type autoimmune disease]
Sequence of quantative events 54: Predicted effect of an increase in antigen production by a certain cell and microcompetition with foreign N-boxes on susceptibility to autoimmune disease.

The two boxed arrows represent the two exogenous events. The boxed arrow next to [Antigen]_{β cell} indicates the transgene induced increase in antigen production by β cells. The boxed arrow next to [p300·GABP·N-box_{TF}] indicates the effect of microcompetition between the GABP latent virus and TF transcription.

As expected, a study (Oldstone 1991⁶⁷⁵) reports that 6% of the RIP-GP and RIP-NP transgenic mice developed hyperglycemia. The pancreatic tissue of these mice showed swollen islets with a group glass appearance (Oldstone 1991, ibid, FIG. 4A). No other exogenous event was necessary to induce disease.

Notes:

1. The rate of trigger apoptosis is this case is a combination of a normal rate of apoptosis with an abnormally high rate of antigen production.

2. To distinguish between mice that developed diabetes and resistant mice, several studies call this case of diabetes a “spontaneous” disease. However, no disease is spontaneous. A disease results from an exogenous event (see discussion in chapter on treatment relating to the difference between the good health and chronic disease equilibria, p 444). In this case, the disease resulted from two exogenous events, a transgene, and an infection with a GABP virus.

(ii) RIP-GP, RIP-NP Transgene+LCMV

Some RIP-GP and RIP-NP transgenic mice develop diabetes, however, most mice do not. Consider resistant mice latently infected with the LCMV. LCMV is a GABP virus. Therefore, the sequence of quantitative events in the preceding section, which holds for all GABP viruses, specifically holds for LCMV. Based on this sequence of quantitative events, RIP-GP and RIP-NP transgenic mice, infected with LCMV, should show diabetes.

As expected, two studies (Ohashi 1991⁶⁷⁶, Oldstone 1991, ibid) showed an increase propensity to develop the autoimmune diabetes (IDDM) in RIP-GP and RIP-NP transgenic mice following infection with the LCMV ARM 53b.

(iii) RIP GP/P14 Double Transgene+CD40

A study (Garza 2000⁶⁷⁷) immunized the RIP-GP/P14 double transgenic mice intravenously with GP33 and FGK45, a rat anti-mouse-CD40 activating antibody. RIP-GP/P14 mice express GP on pancreatic β cells and a LCMV-GP-specific T-cell receptor on T-cells.

CD40 ligation on monocytes/macrophages induces TF cell surface expression. Specifically, treatment of purified monocytes with a stimulating anti-CD40 antibody (BL-C4) strongly induced monocyte procoagulant activity (PCA), which was related to TF expression as shown by flow cytometric analysis (Pradier 1996⁶⁷⁸). Exposure of monocytes/macrophages to either cell membrane isolated from activated CD4+ T-cells (expressing CD40L), or a human rCD40L, increased TF surface expression and activity (Mach 1997, ibid, FIG. 2A, and B, Table). Anti-CD40L mAb blocked induction of TF in response to CD40 ligation. A similar effect on TF expression was observed in vascular smooth muscle cells (SMC) (Schonbeck 2000A, ibid).

CD40 ligation increases TF expression in monocytes/macrophages and, most likely, in dendritic cells. TF expression on monocytes/macrophage and dendritic cells propels backward motility (see chapter on atherosclerosis, p 161). A CD40L deficiency, therefore, should decrease dendritic cell migration to draining lymph node. A study (Moodycliffe 2000⁶⁷⁹) analyzed the in vivo DCs response to skin contact sensitization in CD40 ligand −/− mice. Immunohistochemistry of skin sections in unsensitized CD40 ligand −/− mice revealed no differences in terms of numbers and morphology of dendritic epidermal Langerhans cells (LC) compared to wild-type C57BL/6r mice. However, following hapten sensitization migration of LC out of skin was dramatically decreased and accumulation of DCs in draining lymph nodes substantially diminished in CD40 ligand/mice compared to control (Moodycliffe 2000, ibid, FIG. 2, 3). These observations are consistent with intact forward motility and deficient dendritic cell backward motility.

To conclude, the observations in these studies suggest that CD40 agonists increase TF expression in DCs. Consider the following sequence of quantitative events.
↑[β cell apoptosis]_{Trigger}→↑ [symbol] [Antigen]_{β cell}→↑↑OS→↓↓[p300·GABP·N-box_{TF}]→↑↑ [symbol] [TF_{mRNA}]→↑↑↑TF_{DC} adhesion curve→↑↑↑Skewness of V_{DC} curve→↓↓↓Distance_{DC}→↑↑[Trapped DCs]_{origin} and ↑↑[T-cell]_{lymph mode}→↑↑[T-cells]_{origin}→↑↑[β cell apoptosis]_{T-cell}→↑[β cell-type autoimmune disease/Diabetes]
Sequence of quantative events 55: Predicted effect of an increase in antigen production by β cells and TF transcription on susceptibility to diabetes.

The two boxed arrows indicate the two exogenous events. The boxed arrow next to [TF_{mRNA}] represents the effect of CD40 agonist. This arrow brings the number of arrows to 3, and therefore starts a sequence that ends in disease.

Note that infection with LCMV and treatment with a CD40 agonist added a third arrow to two different variables in the above sequences of quantitative events. Infection with LCMV added a third arrow next to the [p300·GABP·N-box_{TF}] variable, while treatment with a CD40 agonist added a third arrow next to [TF_{mRNA}].

The observations in Garza 2000 (ibid) show that immunization with FGK45, a rat anti-mouse-CD40 activating antibody, produced diabetes in all GP33+CD40 agonist treated mice, unlike immunization with GP33 and a rat polyclonal antiserum as iso-type-matched control (Garza 2000, ibid, 2a). In both groups, the induction of T-cell activation markers and cytotoxic activity were similar. However, GP33+control antibodies produced mild pancreatic infiltration, while GP33+CD40 agonist produced severe insulitis (Garza 2000, ibid, FIG. 2b, c, d).

Garza 2000 (ibid) also reported that administration of GP33 and LPS, another inducer of TF expression, produced diabetes.

The observations in Garza 2000 (ibid) are consistent with the predicted effect of GP33+CD40 agonist immunization, and treatment with GP33+LPS, on immune system dynamics in RIP-GP/P14 double transgenic mice.

Note: The P14 TCR single-transgenic model expresses an LCMV-GP specific T-cell receptor. Garza 2000 (ibid) reports that repeated intravenous administration of the LCMV GP peptide epitope GP33 to P14 transgenic mice induced tolerance and not disease. Peptide administration resulted in upregulation of T-cell activation markers, such as CD69 (Garza 2000, ibid, FIG. 1a). In addition, whereas transgenic T-cells from untreated mice were incapable of lysing peptide pulsed target ex vivo, in vivo peptide treatment induced T-cell cytolytic activity (Garza 2000, ibid, FIG. 1b). Peptide administration induced expansion of T-cells followed by deletions (Garza 2000, ibid, FIG. 1C).

Tissue circulating DCs internalize the administered GP33 peptide. The DCs moderately increase surface antigen expression and costimulation, increase skewness, and eventually migrate to the lymph node where they present the moderate concentration of surface antigen and costimulation to T-cells, leading to activation, proliferation and deletion. Ex vivo treatment with GP33 fails to activate T-cells since activation requires presentation by DCs.

Intravenous administration of GP33 to double transgenic mice (RIP-GP/P14) expressing GP on pancreatic β cells and LCMV-GP-specific T-cell receptor on T-cells surprisingly did not induce diabetes (Garza 2000, ibid, FIG. 2a).

In both models, administration of GP33 activates T-cells. However, since DCs do not increase skewness enough to be trapped in tissue, no homing signal was produced to chemoattract the activated T-cells to the islets.

2. Another study (von Herrath 1997⁶⁸⁰) showed that adoptive transfer of autoreactive CD8+ cytotoxic T-lymphocytes (CTL), which are present in the periphery of RIP-GP or RIP-NP transgenic mice into uninfected transgenic recipients, and are active in vitro and in vivo, rarely induces hyperglycemia or insulitis, despite the cells' ability to home to the islets and induce peri-insulitis. The weak trigger apoptosis in RIP-GP or RIP-NP transgenic mice induces peri-insulitis. However, without LCMV infection, not enough DCs are trapped near the β cells to produce massive insulitis and significant T-cell induced apoptosis. In terms of the two peak model, without excessive skewness, the second peak does not show a large enough diagonal increase. In terms of the sequence of quantitative events, the transfer of autoreactive CD8+ cytotoxic T-lymphocytes, was insufficient to induce 2 arrows next to [T-cells]origin.

(c) Lupus

H8 transgenic mice express the LCMV glycoprotein epitope (GP) 33-41 under control of a major histocompatibility complex class I (MHC I) promoter. Since MHC I is expressed in nearly every cell, H8 mice express and present the GP33 epitope in every cell, including DCs. A study (Ehl 1998⁶⁸¹) adoptively transferred CD8+ T-cells from LCMV T-cell receptor transgenic mice into H8 mice. The transfer led to efficient induction of peripheral tolerance after a period of transient activation and deletion. Next, the study infected H8 mice with LCMV, 1-3 days after T-cell adoptive transfer. Consider the following sequence of quantitative events.
↑[Cellⱼ apoptosis]_{Trigger}→↑ [symbol] [Antigen]_{cell}_{ⱼ}→↑↑OS→↓↓ [symbol] [p300·GABP·N-box_{TF}]→↑↑↑[TF_{mRNA}]→↑↑↑TF_{DC} adhesion curve→↑↑↑Skewness of V_{Dc} curve→↓↓↓Distance_{DC}→↑↑[Trapped DCs]_{origin} and ↑↑[T-cells]_{lymph node}→↑↑↑ [symbol] [T-cells]_{origin}→↑↑↑↑[β cell apoptosis]_{T-cell}→↑↑[Cellⱼ-type autoimmune disease]
Sequence of quantative events 56: Predicted effect of an increase in antigen production, microcompetition with foreigner N-boxes, and number of T-cells near the origin on susceptibility to autoimmune disease.

The boxed arrows indicate the three exogenous events, the one next to [Antigen]_{cell}_{ᵢ} indicates the GP expression in various cells, the one next to [p300·GABP·N-box_{TF}] indicates the effect of microcompetition with LCMV DNA on TF transcription, and the one next to [T-cells]_{origin} indicates the adoptive transfer of CD8+ T-cells. According to the sequence of quantitative events, the additive effect of the three exogenous events should induce tissue wasting and autoimmune disease. Consider the following observations.

As expected, the mice showed signs of wasting 6-8 d postinfection, and 20-40% of the mice died within 12-15 days postinfection, when maintained under specific pathogen-free conditions, and up to 100%, when maintained under non-specific pathogen-free conditions. The remaining mice continued to lose weight, and all died 3-5 months postinfection. Tissue examination revealed CD8+ T-cell infiltration in various organs, including spleen, liver, gut, and skin (Ehl 1998, ibid, FIG. 3). Similar treatment of control mice did not lead to detectable clinical symptoms.

The study also treated H8 mice with 10 μg LPS instead of LCMV infection. LPS increases TF expression on DCs (see chapter on atherosclerosis, p 161), and therefore increases skewness of the DCs backward velocity curve. Consider the following sequence of quantitative events.
↑[Cellⱼ apoptosis]_{Trigger}→↑ [symbol] [Antigen]_{cell}_{ⱼ}→↑↑OS→↓↓[p300·GABP·N-box_{TF}]→↑↑ [symbol] [TF_{mRNA}]→ . . . →↓↓↓Distance_{DC}→↑↑↑T-cell activetion
Sequence of quantative events 57: Predicted effect of an increase in antigen production and increase in TF mRNA on T-cell activation.

The boxed arrow next to [Antigen]_{cell}_{ᵢ} indicates the effect of the transgene on antigen concentration. The boxed arrow next to [TF_{mRNA}] indicates the effect of the LPS treatment. The additive effect of the two exogenous events should induce increase T-cell activation and decrease tolerance. As expected, LPS treatment of H8 mice induced T-cell activation (Ehl 1998, ibid, FIG. 8b, Table 1).

The following table compares RIP-GP and H8 transgenic mice infected with LCMV in terms of DCs surface concentration of the GP33 antigen. In spleen, liver, gut, and skin, internal expression of GP33 in DCs tips the balance from tolerance (or delayed infiltration) in RIP-GP mice to T-cell infiltration in H8 mice (compare cells in table above for same tissue in both mice models). In pancreas, the lack of DCs internal expression of GP33 in RIP-GP mice is probably more than compensated by the increase [Antigen] near pancreatic β cells induced by transfection with RIP-GP (see above).

TABLE 15
Comparison of RIP-GP and H8 transgenic
mice infected with LCMV.

| | RIP-GP | H8 |
|---|---|---|
| Pancreas | (Very) high [Antigen] + | DCs internal GP33 + |
| | LCMV decreased backward | Low tissue renewal + |
| | motility | LCMV decreased backward |
| | | motility |
| Spleen | High tissue renewal + | DCs internal GP33 + |
| | LCMV decreased backward | High tissue renewal + |
| | motility | LCMV decreased backward |
| | | motility |
| Liver | High tissue renewal + | DCs internal GP33 + |
| | LCMV decreased backward | High tissue renewal + |
| | motility | LCMV decreased backward |
| | | motility |
| Gut | High tissue renewal + | DCs internal GP33 + |
| | LCMV decreased backward | High tissue renewal + |
| | motility | LCMV decreased backward |
| | | motility |
| Skin | High tissue renewal + | DCs internal GP33 + |
| | LCMV decreased backward | High tissue renewal + |
| | motility | LCMV decreased backward |
| | | motility |

The concepts presented in this table also predict that in H8 mice, the rate of T-cell infiltration in different tissues is correlated with the rate of tissue renewal.

Systematic lupus erythematosus (also called disseminated lupus erythematosus, lupus, lupus erythematosus and SLE) is a chronic inflammatory autoimmune disease that affects many organs including skin, joints, kidney, heart, lung, and nervous system. At onset, only one organ system is usually involved, however, additional organs may be affected later. A typical observation in lupus patients and animal models is “spontaneous” T-cell activation and organ infiltration.

Consider a latent (persistent) infection of DCs with a GABP virus. Microcompetition between the viral and TF N-boxes increases TF surface expression, and decreases DCs backward motility. According to the excessive skewness/two peak model, the excessive skewness induces pathologies similar to the symptoms observed in lupus patients. The organs affected first are those that show temporary or typical high trigger apoptosis (injured organs or organs with high rates of tissue renewal).

Monocyte/macrophage infection with a GABP virus results in atherosclerosis (see chapter on atherosclerosis, p 161). Both DCs and macrophages originate from CD34+progenitor cells (Hart 1997, ibid, FIG. 3), which are permissive for a GABP viral infection. For instance, Zhuravskaya 1997 (ibid) demonstrated that human cytomegalovirus (HCMV), a GABP virus, persisted in infected bone marrow (BM) CD34+cells (see also, Maciejewski 1999 ibid, Sindre 1996, ibid). According to the proposed model, infection of CD34+cells, therefore, results in both lupus and atherosclerosis. The observed concurrence of lupus and atherosclerosis is well documented. See for instance recent reviews on the issue of accelerated atherosclerosis in systemic lupus erythematosus (Ilowite 2000⁶⁸², Urowitz 2000⁶⁸³). Such observations are consistent with the predicted effects of microcompetition with foreign DNA, and the two peak model.

Another observation explained by the model is the occurrence of hypercoagulation thrombosis in lupus. The infection of CD34+with a GABP virus increases TF expression on circulating monocytes. Such excessive TF expression in lupus was documented in a few studies (see, for instance, Dobado-Berrios 1999⁶⁸⁴). The excessive TF expression increases the probability of coagulation (see also chapter on stroke, p 271, on thrombosis in lupus and other diseases).

(d) Graft Versus Host Disease (GVHD)

Consider a DC migrating “near” pancreatic β cells at a certain average velocity. During the time the DCs spends “near” the cells, it has a certain probability, denoted P, to internalize a certain concentration of β cell antigens, denoted [Antigen]_{β cell}. Now, consider another DC also migrating at the same average velocity. Assume the rate of antigen internalization is independent of DC number in the environment. Then, the probability that, at least one of the cells internalizes [Antigen]_{β cell}, is 2P (the independent assumption does not hold if, for instance, the two DCs co-migrate and end up internalizing a portion of [Antigen]_{β cell} each). Under the independent assumption, an increase in the number of migrating DCs, without a change in other conditions, increases the probability of antigen internalization. Consider, as an alternative situation, one DC migrating at half the original average velocity. Since the time the DC spends near the β cells is twice as long, the probability that the cell internalizes [Antigen]_{β cell} is 2P, the same as the probability of the two DCs migrating at the original average velocity. An increase in the number of migrating DCs, and decrease in average migration velocity of an existing pool of DCs, produces the same effect. Repetitive immunization with H8-DCs is, therefore, equivalent to increase in skewness. Since an excessive skewness induces autoimmune disease, an excessive increase in DC number should also induce autoimmune disease. Consider the following observations.

DCs from H8 mice (H8-DC) constitutively express the GP33 epitope. A single injection of 10⁶H8-DCs (high dose) to RIP-GP transgenic mice resulted in no glycemic change or a transient increase in blood glucose to intermediate levels (15-20 mM), eventually returning to normal levels within a few days (Ludewig 1998⁶⁸⁵, FIG. 1A). A single injection of 10⁵ H8-DCs (intermediate dose) did not result in diabetes. However, repetitive H8-DCs injections of intermediate doses, i.e., three doses of 10⁵ DCs at 6-d intervals (Ludewig 1998, ibid, FIG. 1C), or four doses of 10⁴ DCs at 2-d intervals (Ludewig 1998, ibid, FIG. 1D), resulted in T-cell infiltration (Ludewig 1998, ibid, FIG. 3) and diabetes. 50% of the repetitively immunized mice developed diabetes between day 10 and 14, while 40% developed hyperglycemia by days 18-21. Based on these observations, Ludewig, et al., (1998, ibid) concluded: “the duration of antigenic stimulation by professional APCs, i.e., the integral of CTL activity over time, determines the disease outcome in this model of autoimmune diabetes.”

The observations in Ludewig 1998 (ibid) are consistent with the predicted effect of an increase in the number of DCs on the propensity to develop an autoimmune disease.

Graft-versus-host disease (GVHD) is a complication following allogeneic bone marrow (BM) transplantation (BMT). A typical observation in GVHD patients is spontaneous T-cell activation and organ infiltration. Approximately 50% of patients undergoing allogeneic BMT with related HLA-matched donor develop GVHD.

A study (Fearnley 1999⁶⁸⁶) measured the percentage of DCs present in blood mononuclear cells (MNC) in patients following allogeneic and autologous stem cell transplantation and healthy controls. The mean number of DCs as a percentage of MNC was 0.58%, 0.40% and 0.42%, for patients following allogeneic transplantation showing no GVH symptoms, patients following autologous transplantation, and healthy controls, respectively (p=0.06 for the difference between allogeneic and autologous patients) (Fearnley 1999, ibid, FIG. 3, 6). These results indicate that allogeneic stem cell transplantation increases DCs number, which increases the probability of antigen internalization. In tissues with high normal apoptosis (rapidly renewing tissues), such increase might result in T-cell infiltration and tissue damage.

(e) Vaccination with DCs

Assume a direct relation between the [TF], [CD86], and level of antigen presentation on DCs (denoted [Ag]). Treatment with CD40L, pulsing, apoptosis of tissue of bystander cells, and transfection with a gene expressing an epitope, increases [TF], [CD86], and [Ag]. The increase will be called maturation. Assume that the distribution of the number of DCs expressing [TF], [CD86], and [Ag] is normal. Consider FIG. 107.

Maturation in the figure is represented by a shift of the DCs distribution to higher [TF], [CD86], and [Ag] values. According to the TF propelled backward motility model, there exists a certain level of TF expression, which traps DCs in tissue. The level is marked with a thick line. A cell with lower TF concentration is migration-borne (capable of migrating). A cell with higher TF concentration is trapped.

Consider vaccination with two kinds of cells, less mature and more mature, denoted with solid lines in the figure. Vaccination with the less mature cells induces no trapping. All cells migrate out of tissue. In contrast, vaccination with more mature cells induces cell trapping. Some cells migrate out of tissue, represented by the area under the distribution left of the thick line), while the rest are trapped (the area right of the thick line). Consider the following observations.

A study (Barratt-Boyes 2000⁶⁸⁷) cultured DCs from CD14+ peripheral blood monocytes of rhesus macaques in GM-CSF and IL-4 for 4 days. The cells showed no expression of CD83, the mature DCs marker, moderate expression of the costimulatory molecules CD80, CD86, and CD40, and high levels of MHC class I and II (Barrati-Boyes 2000, ibid, FIG. 1). These cells were designated immature DC. Other cells were cultured for an additional 2 days (total of 7 days) with added CD40L, a known inducer of rapid maturation. The addition of CD40L induced uniform expression of CD83 and high expression of CD80, CD86, and CD40 (Barratt-Boyes 2000, ibid, FIG. 1). These cells were designated mature DCs. The study then labeled the cells with DiD and injected between 2.7×10⁶ and 5.2×10⁶ cells i.d. into anesthetized animals from which cells were derived, in a region lateral to the proximal inguinal lymph node chain. To determine the relative efficiency of immature and mature DCs migration, the site of injection was inspected 36 h after the injection of cells.

FIG. 108 presents the experimental configuration in this study.

According to the figure, many more mature DCs should be trapped in the site of injection, which will induce more severe inflammation reaction at the site. Consider the following observations.

Injection of immature DCs resulted in minor localized acute inflammatory response. No fluorescently labeled cells could be identified at that time. In contrast, injection of mature DCs resulted in a severe acute inflammatory infiltrate at the site of injection in two out of three animals. A large number of fluorescently labeled DCs were detected in the dermis at 36 h in these animals (Barratt-Boyes 2000, ibid, FIG. 8).

Many more DCs are trapped following injection with mature rather than immature cells. Compare the areas to the right of the thick line under the mature and immature curves. In this study, the size of the area representing the trapped DCs, following injection of immature cells, is close to zero.

Notes:

1. 36-hours after injection with immature DCs, the injection site showed no fluorescently labeled cells. However, according to the two peak model, some DCs should be trapped in tissue to produce T-cell infiltrating. This inconsistency between the model and the observation can be resolved if the infiltration T-cells cleared most of the few trapped cells before the 36 hour inspection.

2. The study also reports that, following injection of immature and mature DCs, a portion of the injected cells (0.07-0.12%) reached the lymph node (Barratt-Boyes 2000, ibid, FIG. 7), producing an immune reaction at the injection site. In terms of the figure above, in both cases the area under the curves, left of the thick line, is not empty. Both injections included migration-borne DC. Similar observations are reported in Hermans 2000⁶⁸⁸.

Not all injected DCs migrate to the lymph node. Some cells enter circulation. These DCs can end up in any tissue. According to the discussion above, if enough injected DCs enter circulation over an extended period, the cells might produce an immune reaction in tissues with abnormally high epitope expression, or rapidly renewing tissues. Consider the following observations.

SM-LacZ transgenic mice widely express the β-galactosidase (β-gal) antigen in cardiomyocytes of the right ventricle and in arterial smooth muscle cells. Repetitive treatment of SM-LacZ mice with DCs presenting the β-gal peptide resulted in vascular immunopathology with strong lymphocytic infiltration in small and medium-sized arteries and in the right ventricle (Ludewig 2000⁶⁸⁹). Immunization of SM-LacZ mice with DCs pulsed with an irrelevant peptide produced mild liver infiltration and no anti-β-gal CTL activity. Immunization of non-transgenic mice with DCs presenting the β-gal peptide also produced a mild liver infiltration and no anti-β-gal CTL activity. Naive SM-LacZ mice showed no specific CTL reactivity (Ludewig 2000, ibid, FIG. 2B). Roskrow 1999⁶⁹⁰ reports similar observations of autoimmune disease induced by DCs immunization.

(ii) Studies with TMEV

(a) Conceptual Building Blocks

(i) Persistent Infection in CNS

Theiler's murine encephalomyelitis viruses (TMEVs) are members of the genus Cardiovirus in the family Picornaviridae. These viruses can be divided into two groups based on their neurovirulence characteristics following intracerebral (i.e.) inoculation of mice. Highly virulent strains, such as GDVII virus, cause rapidly fatal encephalitis. The less virulent strains, such as BeAn and DA, show at least a 10-fold decrease in the mean 50% lethal dose (LD₅₀) compared to the virulent strains. Moreover, they can establish a persistent infection in the central nervous system (CNS).

(ii) GABP Virus

The following observations suggest that all three TMEV strains, GDVII, BeAn and DA are GABP viruses. The 5′ UTR of all three strains includes 9 N-boxes. The 5′ UTR of all three strains includes a pair of N-boxes (positions (−129, −123) and (121, −115), or positions (935, 941) and (943, 949) when numbered according to the BeAn sequence). It is interesting that the pair in GDVII is different than the pair in BeAn and DA. In GDVII, the pair of N-boxes (underlined) is CTTCCGCTCGGAAG while the pair in BeAn and DA is CTTCCTCTCGGAAG. The GDVII pair is symmetrical while the pair in BeAn and DA is not. The asymmetry in BeAn and DA might result in decreased affinity to GABP, and therefore a decreased rate of transcription initiation. This interpretation is consistent with the following observations.

In a series of experiments, a study (Lipton 1998⁶⁹¹) attempted to Identify the DNA sequences associated with the difference in the virulence of these strains. In these experiments, the study constructed recombinant TMEVs by exchanging corresponding genomic regions between GDVII and BeAn. One such recombinant virus is Chi 5L in which the (933, 1142) BeAn sequence replaces the original GDVII sequence. Inoculation of the recombinant virus Chi 5L into mice by the intracerebral route showed attenuated neurovirulence. The LD₅₀ value for Chi 5L was ≧7.5×10⁵ in comparison to 10 for GDVII (Lipton 1998, ibid, table 1). The replacement of the original GDVII pair of N-boxes with the BeAn pair decreased virulence.

(b) Demyelination (Multiple Sclerosis)

As with many other viruses, TMEV infection spreads from cell to cell. However, the identity of infected cells, and the order of viral cell-to-cell spread determine the clinical outcome. Consider an infection with a BeAn and DA virus. The first cells infected in the nervous system are neurons. The infection results in apoptosis with cell debris internalized by surveilling macrophages, increase in skewness, trapping a few cells leading to T-cell infiltration. These events are characteristic of the acute phase, which terminates when the neuronal infection is cleared, inflammation in gray matter subsides, and neuron apoptosis returns to normal levels. However, during the acute phase the virus spreads from neurons to some infiltrating macrophages thereby establishing a persistent infection. The infection increases surface TF expression, increases skewness of macrophages, and traps some cells in the white matter. Since infection is not lytic, trapped macrophages continue to internalize Schwann cell/oligodendrocyte debris or apoptotic cells produced in normal cell turnover or during myelin damage. The internalized myelin is processed and presented on the cell surface. Loaded macrophages release cytokines, which function as a homing signal to T-cells and new infiltrating macrophages. Both trapped macrophages and Schwann cells/oligodendrocytes present myelin on their surface MHC. Infiltrating T-cells bind the presented myelin, and destroy the antigen presenting cells. The result of such destruction is demyelination. The observations in the following studies are consistent with this sequence of quantitative events.

Tsunoda 1997⁶⁹² showed that the first cells infected in the nervous system are neurons, and that the initial limited inflammation in the gray matter subsides concurrently with the decline in neuronal apoptosis. Ha-Lee 1995⁶⁹³ reports similar observations.

According to Lipton 1995⁶⁹⁴, viral antigens were first detected in white matter on day 14 post inoculation. On days 14 and 22, viral antigens were occasionally seen within long stretches of axons extending from the gray matter into anterior white matter (Lipton 1995, ibid, FIG. 2A). MOMA-2-positive cells (MOMA-2 is a monoclonal antibody to macrophages), some of which contained viral antigens, were observed in close proximity to infected axons (Lipton 1995, ibid, FIG. 2A). The observation suggests that TMEV leaves the gray matter by axonal spread. TMEV is released from the axoplasm as motor neurons, and then secondarily infects macrophages in the white matter. The fact that motor neurons are the principle virus target in the acute gray matter phase of infection, and the predominantly anterolateral location of viral antigen-positive cells in the white matter on days 14, 22, and 29, support this conclusion. Increasing numbers of viral antigen-positive, MOMA-2-positive cells, appeared in the thoracic cord white matter between days 14 and 49, and remained at the increased level of infection until day 73. However, only a small fraction of MOMA-2-positive cells contained viral antigens during this period (Lipton 1995, ibid, FIG. 2B). The early infiltration, and apparent spread; of these cells from anterior to posterior in the spinal cord, with a tendency for viral antigen-positive cells to be found at the periphery of advancing; edges of lesions (Lipton 1995, ibid, FIG. 3), also supports this conclusion. Based on these observations, Lipton, et al., (1995, ibid) concluded that at least some of the MOMA-2-positive cells have a hematogenous origin, and, that infection occurs upon entry of these cells into the CNS.

Miller 1997⁶⁹⁵ reports the temporal appearance of T-cell response to viral and known encephalitogenic myelin epitopes in TMEV-infected SJL/J mice. Clinical signs, which appear approximately 30 days after infection, display chronic progression with 100% of the animals affected by 40-50 days postinfection. Ultraviolet light (UV)-inactivated TMEV produced T-cell proliferation in spleen of infected mice, at day 33 postinfection, concomitant with onset of clinical signs, and at day 87. In contrast, at 33 d postinfection, the major encephalitogenic, epitope on myelin proteolipid protein (PLP139-151 and PLP178-191), and myelin-basic protein (MBP84-0.104), did not produce T-cell proliferation in spleen, cervical or pooled peripheral lymph nodes. However, a response to PLP139-151 was observed in all lymphoid compartments at day 87 postinfection. Similar temporal observations “are associated with the appearance of CD4+ Th1-mediated delayed-type hypersensitivity (DTH) responses. The immunodominant TMEV VP2 70-86 epitope produced DTH at all times tested. In contrast, the PLP139-151 epitope first produced DTH only at day 52, persisting through day 81 postinfection (Miller 1997, ibid, FIG. 1C). Assessment of DTH in a larger panel of encephalitogenic myelin epitopes during late chronic disease (164 days postinfection), showed persistence of peripheral T-cell reactivity to both VP2 70-86 and PLP178-151, and appearance of responses to multiple, less immunodominant myelin epitopes including PLP56-70, PLP178-191, and the immunodominant myelin oligodendrocyte glycoprotein epitope (MGO92-106) (Miller 1997, ibid, FIG. 1d). The study calls this observation “epitope spreading,” and defines it as the process whereby epitopes distinct from, and non-cross-reactive with an inducing epitope become major targets of an ongoing immune response. The longer macrophages are trapped in white matter, the higher the concentration of presented epitopes on cell surfaces. Since “rare” epitopes require longer macrophage residence time to accumulate at high enough concentrations, the reported epitope spreading indicates abnormally long macrophage residence time, or abnormally high macrophage trapping.

(2) Human Studies

In addition to the autoimmune diseases mentioned in the sections above, studies reported observations on other autoimmune diseases, such as asthma, rheumatoid arthritis, thyroiditis, and inflammatory bowel disease. These observations are also consistent with the predicted effects of excessive skewness on the dynamics of the immune system. For instance, studies in animal models of asthma showed that DCs collect antigens in the airways, upregulate [Ag] and [B7], migrate to the thoracic lymph nodes where they present the antigens to T-cells (Vermaelen 2001⁶⁹⁶). Other studies showed that DCs are essential for development of chronic eosinophilic airway inflammation in response to inhaled antigen in sensitized mice (Lambrecht 2000A⁶⁹⁷, Lambrecht 2000B⁶⁹⁸, Bertorelli 2000⁶⁹⁹, Lambrecht 1998⁷⁰⁰). Additional studies showed the significant role of B7 in allergic asthma (Mathur 1999⁷⁰¹, Haczku 1999⁷⁰², Padrid 1998⁷⁰³, Keane-Myers 1998⁷⁰⁴). Similar observations were reported in rheumatoid arthritis (see, for instance, Balsa 1996⁷⁰⁵, Liu 1996⁷⁰⁶), and thyroiditis (see, for instance, Watanabe 1999⁷⁰⁷, Tandon 1994⁷⁰⁸).

The following sections present several predicted effects of excessive skewness, and compare the predicted effects with observed dynamics of many autoimmune diseases, as reported in studies with human patients.

(a) Early T-Cell Infiltration

According to the excessive skewness model of autoimmune disease, T-cell infiltration precedes permanent tissue cell destruction.

As expected, T-cell infiltration, or insulitis, was extensively reported in pre-diabetic and recent-onset diabetic patients, see, for instance, Signore 1999⁷⁰⁹, Foulis 1991⁷¹⁰, Foulis 1984⁷¹¹. Similar observations are reported in multiple sclerosis (Bitsch 2002⁷¹², Brown K A 2001⁷¹⁰, Pouly 1999⁷¹⁴), psoriasis (Bata-Csorgo 1995⁷¹⁵, Baadsgaard 1990⁷¹⁶), lupus (Hoffman 2001⁷¹⁷, Chan 1999⁷¹⁸), asthma (Trautmann 2002⁷¹⁹, Poston 1992⁷²⁰), rheumatoid arthritis (Strober 1990⁷²¹), and thyroiditis (Stassi 2001⁷²², Eguchi 2001⁷²³).

The observations in these studies are consistent with the predicted effect of excessive skewness on timing of T-cell infiltration.

(b) B7 in Trapped DCs

According to the excessive skewness model of autoimmune disease, trapped antigen-presenting cells (APCs), specifically DCs and macrophages, should show high expression of B7.1 (CD80) and/or B7.2 (CD86).

As expected, infiltrating macrophages in brain sections from multiple sclerosis (MS) patients showed significant B7 immunoreactivity, in contrast to normal brains that showed no B7 immunoreactivity (De Simone 1995⁷²⁴) Another study (Windhagen 1995⁷²⁵) found B7.1 staining in plaque from MS patients localized predominantly to lymphocytes in perivenular inflammatory cuffs, and B7.2 staining predominantly on macrophages in inflammatory infarcts.

A study (Ohki 1997⁷²⁶) measured the expression of co-stimulatory molecules in atopic dermatitis (AD) and psoriasis (Ps) patients. As expected, B7.2 and B7.1 were detected on dendritic-shaped cells not only in the epidermis but also in the dermis in the inflammatory lesions of atopic dermatitis (n=12). B7.2 was expressed in all cases (100%), while B7.1 was expressed in only five cases (42%). These molecules were not detected in normal control subjects (n=8). Neither B7.1 nor B7.2 was detected on keratinocytes. Stronger expression of B7.2 over B7.1 was also observed in psoriasis vulgaris (n=11), and contact dermatitis (n=7).

Note that since DCs increase B7 expression while migrating out of tissue, in the case of Langerhans cells while migrating from epidermis to dermis and then lymph vessel, B7 expression on Langerhans cells in dermis should be higher than cells in epidermis. As expected, Ohki 1997 (ibid) showed an increase in B7 expression in Langerhans cells in the dermis compared to the epidermis.

As expected, a study (Agea 1998⁷²⁷) showed overexpression of B7.2, and to a lesser extent, B7.1 on alveolar macrophages (AM) from asthmatics patients compared to normal subjects, untreated patients with pulmonary sarcoidosis (PS), or individual with extrinsic allergic alveolitis (EAA). Similar observations are reported in Balbo 2001⁷²⁸, Burastero 1999⁷²⁹, and Hofer 1998⁷³⁰.

Studies also showed increased expression of B7 molecules on APCs in inflammatory bowel disease (IBD) (Rogler 1999⁷³¹, Rugtveit 1997⁷³², Hara 1997⁷³³, in Crohn's disease, Liu Z X 1997⁷³⁴, in Crohn's disease), rheumatoid arthritis (Balsa 1996, ibid, Liu 1996, ibid, Shimoyama 1999⁷³⁵, Thomas 1996⁷³⁶), Systemic lupus erythematosus (Denfeld 1997⁷³⁷, allergic contact dermatitis (Simon 1994⁷³⁸), and thyroiditis (Tandon 1994, ibid).

Note that in Denfeld 1997 (ibid), the dermal and epidermal APCs showed increased expression of B7 only in lesional sections where DCs are trapped, and usually when opposing CD8+ T-cells.

The observations in these studies are consistent with the predicted effect of excessive skewness on B7 expression.

(c) Chemokines

According to the excessive skewness model of autoimmune disease, trapped DCs express chemokines, including MIP-1α, MIP-1β, and RANTES. Therefore, damaged tissue, and specifically trapped macrophages, should show high expression of these chemokines.

A study (Boven 2000⁷³⁹) measured expression of the CC chemokines MIP-1α, MIP-1β, and RANTES in brain tissue from MS patients using reverse transcriptase-polymerase chain reaction (RT-PCR) techniques. As expected, both MIP-1β and RANTES were significantly elevated. In addition, MIP-1α was also increased, although not significantly. Immunohistochemistry revealed that MIP-1α and MIP-1β immunoreactivity was predominantly found in perivascular and parenchymal macrophages containing myelin degradation products.

As expected, studies also showed increased expression of chemokines in asthma (Alam 1996⁷⁴⁰, Hsieh 1996⁷⁴¹, Holgate 1997⁷⁴²), inflammatory bowel disease (Banks 2003⁷⁴³, in colonic mucosal biopsies of both ulcerative colitis and Crohn's disease patients, Uguccioni 1999⁷⁴⁴, in colonic biopsies of ulcerative colitis patients, Vainer 1998⁷⁴⁵), psoriasis and atopic dermatitis (Hatano 1999⁷⁴⁶), rheumatoid arthritis (Katrib 2001⁷⁴⁷, Volin 1998⁷⁴⁸, RANTES in tissue macrophages, al-Mughales 1996⁷⁴⁹, Hosaka 1994⁷⁵⁰), and Sjogren's syndrome (Cuello 1998⁷⁵¹).

Note: Katrib 2003⁷⁵² also showed decreased expression of chemokines in RA remission.

The observations in these studies are consistent with the predicted effect of excessive skewness on chemokine expression.

(d) Lipoprotein(a)

A biological function of Lp(a) is to decrease skewness (see chapter on atherosclerosis, p 161). Therefore, patients with an autoimmune disease should show an increase in Lp(a) levels.

As expected, studies reported increased Lp(a) levels in diabetes (Matteucci 2000⁷⁵³ Kronenberg 1999B, ibid, Serban 1995⁷⁵⁴), rheumatoid arthritis (Busso 2001⁷⁵⁵ (higher than osteoarthritis), Asanuma 1999⁷⁵⁶, Lee 2000⁷⁵⁷, Park YB 1999⁷⁵⁸), lupus (Sari 2002⁷⁵⁹), antiphospholipid antibody syndrome (Yamazaki 1994⁷⁶⁰, Atsumi 1998⁷⁶¹), thyroiditis (specifically, hypothyroidism, see note below) (Tzotzas 2000⁷⁶², Kung 1995A⁷⁶³, Klausen 1992⁷⁶⁴ (irresponsive to T4 treatment), Engler 1993⁷⁶⁵, de Bruin 1993⁷⁶⁶), inflammatory bowel disease (van Bodegraven 2001⁷⁶⁷, Koutroubakis 2001⁷⁶⁸, in Crohn's disease, Kawabata 1997⁷⁶⁹, in ulcerative colitis), and psoriasis and atopic dermatitis (Uyanik 2002⁷⁷⁰, Rocha-Pereira 2001⁷⁷¹, Camp 1999⁷⁷², Cimsit 1998⁷⁷³ Seckin 1994⁷⁷⁴)

Notes:

1. In addition to diabetic patients, Matteucci 2000 (ibid) showed increased levels of Lp(a) in non-diabetic siblings and non-diabetic parents of type 1 diabetic subjects (Matteucci 2000, ibid, Table 1). Assume infection among family members (for instance, through congenital infection). Then, family members of type I diabetic patients should show an increased probability of harboring a latent infection with a GABP virus, and therefore, an increase in Lp(a) levels, as observed in Matteucci 2000 (ibid).

2. Kronenberg 1999B (ibid) showed an increase in Lp(a) only in short term diabetes, which was related to a survival effect (see study details in the chapter on atherosclerosis, p 161).

3. Thyroxin (T4) is an ERK agent (Kozawa 2001⁷⁷⁵, Lin 1999⁷⁷⁶) Therefore, patients showing hyperthyroidism (excess T4) should show decreased Lp(a). Excessive ERK phosphorylation produces the opposite effects of microcompetition with foreign DNA. As expected, decreased plasma Lp(a) was observed in Hoppichler 1995⁷⁷⁷, Kung 1995B⁷⁷⁸, Engler 1993 (ibid), and de Bruin 1993 (ibid). Note that hyperthyroidism in Graves' disease patients is associated with weight loss, as expected (microcompetition with foreign DNA results in weight gain, therefore, excessive ERK phosphorylation should lead to weight loss), although patients experience an increase in appetite.

(e) Tenascin-C (TNC)

A biological function of TNC is to decrease the steepness of the fibronectin gradient, which is equivalent to a decrease in skewness (see chapter on atherosclerosis, p 161). Therefore, patients with an autoimmune disease should show increased TNC.

As expected, studies reported increase in TNC in diabetes (Loots 1998⁷⁷⁹ prolonged TNC expression and significant increase in number of macrophages in the edges of wounds, Spirin 1999⁷⁸⁰, in retinas of diabetic patients without retinopathy), asthma (Armin 2000⁷⁸¹, Karjalainen 2000⁷⁸², Laitinen 1997⁷⁸³, Laitinen 1996⁷⁸⁴), Hashimoto Thyroiditis (Back 1997⁷⁸⁵), Sjögren's syndrome (Amin 2001⁷⁸⁶), inflammatory bowel disease (Geboes 2001⁷⁸⁷, both ulcerative colitis and Crohn's disease, especially in areas of ulceration, Riedl 2001⁷⁸⁸ (in serum), Riedl 1998⁷⁸⁹, also in neoplastic disease of the large bowel, Riedl 1997⁷⁹⁰), psoriasis and atopic dermatitis Latijnhouwers 1998⁷⁹¹, tenascin-C remained abundant after clinical remission of lesions, Schalkwijk 1991⁷⁹²), and rheumatoid arthritis (Salter 1993⁷⁹³, Chevalier 1994⁷⁹⁴).

Notes:

1. Karjalainen 2000 (ibid) also showed increased TNC expression in the subepithelial basement membrane in endobronchial biopsy specimens of the proximal airways collected from 40 elite, competitive, non-asthmatic skiers compared to controls (Karjalainen 2000, ibid, FIG. 6). Assume that strenuous training in low temperatures with repeated inhalation of cold air induces substantial trigger apoptosis. According to the two peak model, competitive skiers should show excessive skewness, T-cell induced apoptosis, and tissue damage. As a protective reaction against the excessive number of trapped macrophages, the immune system should increase TNC expression, consistent with the observations reported in Karjalainen 2000 (ibid).

2. Also consistent with the predicted effects of excessive skewness, Karjalainen 2000 (ibid) showed an increase in number of macrophages in bronchial biopsy specimens from both asthmatic patients and competitive skiers compared to controls (Karjalainen 2000, ibid, FIG. 1)

The observations in these studies are consistent with the predicted effect of excessive skewness of TNC expression.

(f) Puberty

Skewness of the monocytes velocity curve peaks during puberty (see chapter on atherosclerosis, p 161). Assume cell shows a fixed rate of apoptosis before and after puberty, then, the rate of onset of cells-type autoimmune disease should show a local peak during puberty.

As expected, studies showed a local peak in onset of diabetes (Li X H 2000⁷⁹⁵, relative risk 1.0, 2.3 and 3.6 for age group 0-4,5-9 and 10-14 years, respectively, Huen 2000⁷⁹⁶, incidence rates of 0.9, 1.5, and 1.7 per 100,000/year for age group 0-4,5-9, and 10-14 years, respectively, Karjalainen 1989⁷⁹⁷, Green 1983⁷⁹⁸), and psoriasis (Swanbeck 1995⁷⁹⁹), during puberty.

Note: Existence of other local peaks, at different age groups, is not inconsistent with the model, since the other age groups might be associated with increased rate of apoptosis.

(g) Onset of Th2 vs. Th1 Diseases

The effectiveness of the immune system deteriorates with age (see reviews Khanna 1999⁸⁰⁰, Ginaldi 1999⁸⁰¹), which might explain the increased incidence of infectious diseases in the aged. Consider an individual harboring a persistent infection of a GABP virus in DCs (for instance, cytomegalovirus). At every age, the balance between two forces, the virus drive to replicate and the capacity of the immune system to control or clear the infection, determines the viral genome copy number present in infected cells. A decline in immune system effectiveness, therefore, increases viral genome copy number. Consistent with that conclusion, Liedtke 1993⁸⁰² showed an increase in the prevalence of HSV-1 neuronal latency with age.

An increase in viral genome copy number intensifies microcompetition between cellular genes and viral DNA, which decreases average DC velocity, and increases surface expression of [Ag] and [B7] on DCs reaching the draining lymph node. The increase in [Ag] and [B7] increases [DC·T], which increases the probability of Th1 vs. Th2 differentiation. This argument predicts a decline in Th2 and increase in Th1 autoimmune diseases with age. Specifically, the argument predicts earlier onset of Th2 compared to Th1 autoimmune diseases in the same patient. Consider the following observations.

Atopic dermatitis (AD) is a Th2 disease, while psoriasis (Ps) is a Th1 disease. A study (Beer 1992⁸⁰³) collected information on the onset of AD and Ps in patients attending a dermatology clinic. Information was available on 983 patients, 224 with AD, 428 with Ps, 45 with both AD and Ps, and 286 controls. 16.7% of the AD patients also had Ps, and 9.5% of Ps patients had AD. In consecutive occurrences, Ps generally followed AD. Out of the 45 patients with both AD and Ps, 26 patients had an onset of AD first and Ps later in life (average age=10 and 26, respectively), 9 subjects (all children) had simultaneous onset of AD and Ps, and 1 patient had first onset of Ps at the age of 16, followed by AD+Ps at the age of 18, and return to Ps. As predicted, the observations showed earlier onset of AD, a Th2 disease, compared to Ps, a Th1 disease.

(h) Infection with GABP Viruses

A persistent infection of DCs with a GABP virus increases the probability of developing an autoimmune disease. Moreover, an increase in viral load should exacerbate the disease. Consider the following observations.

To detect active infection, a study (Asadullah 1999⁸⁰⁴) compared the antigen expression of cytomegalovirus (CMV), a GABP virus, in peripheral blood mononuclear cells (PBMC) from psoriatic patients (n=30) and healthy volunteers (n=65). The results showed higher CMV antigenaemia in psoriasis (43%) compared with healthy laboratory staff (12%, p<0.01) and blood donors (6%, p<0.001)

Another study (Steigleder 1986⁸⁰⁵) reports the development of psoriasis vulgaris in four patients suffering from immune deficiency related to HTLV III, a GABP virus. The psoriasis was extensive, exudative, and almost refractory to therapeutic approaches. The bulk of dermal infiltrating mononuclear cells were CD8+ T lymphocytes.

HIV is a GABP virus. According to a recent review (Mallon 2000⁸⁰⁶), psoriasis occurs with at least undiminished frequency in HIV-infected individuals.” Moreover, according to the study, “It is paradoxical that, while drugs that target T lymphocytes are effective in psoriasis, the condition should be exacerbated by HIV infection.” See also the Montazeri 1996⁸⁰⁷ review. Another study reported clinical improvement of HIV-associated psoriasis in parallel with a decrease in HIV viral load induced by effective antiretroviral therapy (Fischer 1999⁸⁰⁸).

Note: In addition to psoriasis, infections with HIV resulted in other autoimmune diseases, such as inflammatory bowel disease (Olsson 2000⁸⁰⁹).

The observations in these studies are consistent with the predicted effects of an infection with a GABP virus on autoimmune disease.

(i) Other Viral Infections

Coxsackie B4 virus infects pancreatic β-cells inducing limited β cell death (Roivainen 2000⁸¹⁰). Limited β-cell destruction does not result in diabetes. However, according to the two peak model, “trigger apoptosis” results in T-cell infiltration. According to the excessive skewness model of autoimmune disease, Coxsackie B4 infection in individuals harboring a GABP virus might result in diabetes. Consistent with this prediction, some recent studies found a strong association between Coxsackie B4 virus infection and onset of insulin-dependent diabetes mellitus in humans (Andreoletti 1998⁸¹¹, Andreoletti 1997⁸¹², Frisk 1997⁸¹³, Clements 1995⁸¹⁴). If Coxsackie B4 is a GABP virus, and can infect DCs, the cellular events resulting from a Coxsackie B4 viral infection resemble the events of a TMEV infection (see above).

d) Other Excessive Skewness Exogenous Events

(1) Smoking

Since smoking also increases skewness, it should show effects similar to infections with a GABP virus. For instance, smoking should increase number of trapped macrophages, T-cell infiltration, and tissue damage. See details in section on smoking in chapter on atherosclerosis, p 254.

As expected, a study (Amin 2003⁸¹⁵) showed an increase in number of inflammatory cells, specifically macrophages, in airways mucosa of asymptomatic smokers compared to non-smoking subjects. The study also showed increased thickness of the tenascin layer and a decrease in the integrity of the epithelial layer in smokers compared to non-smoking subjects. In smokers, the results showed an inverse relation between the number of macrophages and epithelial integrity.

The observations in Amin 2003 (ibid) are consistent with the predicted effects of excessive skewness on the dynamics of the immune system.

e) Treatment

A treatment is an exogenous event. Many treatments of autoimmune diseases are currently available. The following sections present the predicted effects of a few treatments, as examples, and compare the predicted effects with reported observations.

(1) Anti-CTLA-4

Increase in CTLA4Ig decreases [DC·T] (see function above). As a result, T-cell induced apoptosis decreases, which decreases inflammation (DC infiltration, T-cell infiltration, etc.). Consider the following observations.

Abrams 2000⁸¹⁶ administered to patients with psoriasis vulgaris four intravenous infusions of the soluble chimeric protein CTLA4Ig (BMS-188667) in a 26-wk, phase I, open label, dose escalation study. Clinical improvement was associated with decreased cellular activation of lesional T-cells and DCs. Concurrent decreases in B7.1 (CD80) and B7.2 (CD86) were detected on lesional DCs, which also decreased in number within lesional biopsies. Skin explant experiments suggested that these alterations in activated or mature DCs were not the result of direct toxicity of CTLA4Ig for DCs. Based on these observations, Abrams, et al., (2000, ibid) concluded: “this study highlights the critical and proximal role of T-cell activation through the B7-CD28/CD152 costimulatory pathway in maintaining the pathology of psoriasis, including the newly recognized accumulation of mature DCs in the epidermis.”

(2) Fluticasone Propionate (FP)

Fluticasone propionate (FP) decreases NF-κB activation (Cazes 2001⁸¹⁷, Jaffuel 2000⁸¹⁸), which decreases TF expression, decreases skewness, and should decrease the number of DCs in inflammation.

Note: In a small sample size, Hart 2000⁸¹⁹ showed no significant change in NF-κB activation in alveolar macrophages from asthmatic patients following treatment with FP.

As expected, treatment with inhaled FP decreased the number of DC in asthma (Bocchino 1997⁸²⁰, Lawrence 1998⁸²¹, see also a review Johnson 1998⁸²²), and treatment with topical FP decreased the number of the antigen-presenting ‘Langerhans’ cells in nasal mucosa of allergic rhinitis patients (Fokkens 1997⁸²³) (Note: there was no effect on the number of macrophages). See also Nelson 1995⁸²⁴.

Treatment with other corticosteroids, such as beclomethasone dipropionate, also decreased the excessive number of DCs in asthma (Moller 1996⁸²⁵) (Jaffuel 2000, ibid, showed that beclomethasone dipropionate decreases NF-κB activation).

8. Obesity

a) Background

(1) The Obesity Epidemic

The incidence of obesity (defined as body mass index (BMI)≧30 kg/m²) in the United States increased from 12.0% in 1991 to 17.9% in 1998. The increase was observed in all states, in both sexes, across age groups, races and education levels, and regardless of smoking status (Mokdad 1999⁸²⁶).

(2) Three Conjectures About the Cause

The scientific literature includes three “classical” conjectures about the cause of the obesity epidemic: increased energy intake, decreased energy expenditure, and genetic mutation. Despite their wide spread acceptance, these conjectures are inconsistent with existing observations.

(a) Increased Energy Intake (“Too Much Food”)

Many large-scale studies refute the belief that increased energy intake is the cause of obesity. The USDA Nationwide Food Consumption Survey 1977-1988 collected data from over 10,000 individuals. The survey revealed that during the study period, the average total energy intake in the United States actually decreased by 3% in women and 6% in men. Moreover, during the period average fat intake decreased from 41% to 37%. Despite the decreased energy and fat intake, the prevalence of obesity increased (Weinsier 1998⁸²⁷).

An even larger study reported similar results based on pooled data from NHANES II and III, the USDA Nationwide Food Consumption Survey, the Behavioral Risk Factor Survey System, and the Calorie Control Council Report (Heini 1997⁸²⁸). The data revealed a 31% increase in the prevalence of overweight from 25.4% during 1976-1980 to 33.3% during 1988-1991; At the same time, the average total daily calorie intake per capita decreased 4% from 1,854 kcal to 1,785 kcal, with similar trends in men and women. Moreover, the average fat intake, adjusted for total calories, decreased 11% from 41.0% to 36.6%. Concomitant with these changes there was a substantial rise, from 19% in 1978 to 76% in 1991, in the proportion of the US population consuming low-calorie products. According to Heini and Weinsier: “decreased fat and calorie intake and frequent use of low-calorie food products have been associated with a paradoxical increase in the prevalence of obesity.” Similar surveys conducted in Great Britain corroborate these studies.

(b) Decreased Energy Expenditure (“Too Little Exercise”)

Many have turned their attention to decreased physical activity as an alternative explanation for the obesity epidemic, however the data disproves the explanation as well. In recent years, several population surveys have shown unchanging levels of physical activity among Americans. For example, in the Behavioral Risk Factor Survey System, which included 30,000 to 80,000 individuals annually, the prevalence of obesity increased from 12% to 17.9% between 1991 and 1998, yet physical inactivity did not change substantially (Heini 1997, ibid).

(c) Genetic Mutation

Genetic mutation offers an attractive explanation of the observed increase in the incidence of obesity. However, a significant change in the human gene pool requires many generations. A genetic explanation for the increase in obesity implies that the human gene pool has changed over a single generation. “Although research advances have highlighted the importance of molecular genetic factors in determining individual susceptibility to obesity, the landmark discoveries of leptin, uncoupling proteins and neuropeptides involved in body weight regulation, cannot explain the obesity epidemic” (Hill 1998⁸²⁹). “The fact that the increased rates of obesity have been observed within the last two decades has been viewed as evidence that genetic factors cannot be held responsible” (Hebebrand 2000⁸³⁰). “Genes related to obesity are clearly not responsible for the epidemic of obesity because the gene pool in the United States did not change significantly between 1980 and 0.1994” (Koplan 1999⁸³¹).

b) Microcompetition with Foreign DNA

(1) Cellular GABP Regulated Genes and Obesity

The following sections provide evidence that microcompetition between DNA of GABP viruses and cellular GABP regulated genes increases susceptibility to obesity.

(a) Transitive Deduction

The logical principle of transitive deduction can be defined as follows:
IF (A→B)AND(B→C)
THEN (A→C)

If A leads to B, and B leads to C, then A leads to C. The principle of transitive deduction can be extended to any number of steps.

Transitive deduction, also called in logical literature transitive entailment, or cut (the name is associated with the “cut” of the intermediate B), is a fundamental principle of logics. Consider Gabbay 1994⁸³²: “Cut is a very basic rule in traditional logical systems and can be found in one form or another in each one of them.” Note that in Elements of Biology, Weisz stated: “Deductive logic is used extensively by scientists to obtain predictions from hypotheses. . . . Most scientists are so accustomed to deductive reasoning that formal construction of ‘if . . . then . . . ’ statements is unnecessary in setting up experiments” (Weisz 1965⁸³³). In logical literature the above form of transitive deduction is called unitary cut.

(b) Human Metallothionein-II_{A} Gene (hMT-II_{A})

(i) hMT-II_{A} is a Foreign N-box-Suppressed Gene

Microcompetition between foreign N-boxes and the promoter of the human metallothionein-II_{A} (hMT-II_{A}) gene decreases hMT-II_{A} transcription gene (see chapter on microcompetition, p 128).

Symbolically,
↑[N-boxᵥ]→↓[mRNA_{hMT-HA}]
Sequence of quantative events 58: Predicted effect of foreign N-boxes on human metallothionein-II_{A} mRNA levels.

(ii) MT-I or MT-II Null Mutants and Weight Gain

Mice with mutated MT-I and MT-II genes are apparently phenotypically normal, despite decreased expression of the metallothionein genes. The disruption shows no adverse effect on their ability to reproduce and rear offspring. However, after weaning, the MT-null mice consume more food, and gain more weight at a higher rate compared to controls. The majority of adult male mice in the MT-null colony show moderate obesity (Beattie 1998⁸³⁴).

Symbolically,
↓[mRNA_{MT-HA}]→↑Weight
Sequence of quantative events 59: Predicted effect of human metallothionein-II_{A} mRNA on body weight.

(iii) Logical Summary

According to the principle of transitive deduction:
If (↑[N-boxᵥ]→↓[mRNA_{hMT-HA}]) AND (↓[mRNA_{MT-HA}]→↑Weight) Then (↑[N-boxᵥ]→↑Weight)

Since microcompetition with foreign N-boxes decreases metallothionein gene transcription, and since decreased metallothionein gene transcription increases body weight, microcompetition should increase body weight.

(iv) MT-I or MT-II Null Mutants and Hyperleptinemia

Mice with mutated MT-I and MT-II genes also showed high plasma leptin levels (Beattie 1998, ibid). Symbolically,
↓[mRNA_{MT-HA}]→↑[Leptinₚₗₐₛₘₐ]
Sequence of quantative events 60: Predicted effect of metallothionein-II_{A} mRNA on plasma leptin.

(v) Logical Summary

According to the principle of transitive deduction,
If (↑[N-boxᵥ]→↓[mRNA_{hMT-HA}]) AND (↓[mRNA_{MT-HA}]→↑[Leptinₚₗₐₛₘₐ]) Then (↑[N-boxᵥ]→↑[Leptinₚₗₐₛₘₐ])

Since microcompetition with foreign N-boxes decreases metallothionein gene transcription, and since decreased metallothionein gene transcription increases leptin plasma levels, microcompetition should result in hyperleptinemia (see more on microcompetition and hyperleptinemia in chapter on signal resistance, p 340).

(c) Hormone Sensitive Lipase Gene (HSL)

Hormone sensitive lipase gene (HSL, Lipe, EC 3.1.1.3) is an intracellular neutral lipase highly expressed in adipose tissue. HSL is the rate-limiting enzyme in triacylglycerol and diacylglycerol hydrolysis, and mediates cholesterol ester hydrolysis to generate free cholesterol in steroidogenic tissues and macrophages.

(i) HSL is a Foreign N-box-Suppressed Gene

(a) GABP

Of the dozens of known ETS factors, only GABP, as a tetrameric complex, binds two N-boxes. Typically, the N-boxes are separated by multiples of 0.5 helical turns (HT). Consider the examples Yu 1997⁸³⁵, FIG. 1.

The region from −780 bp 5′ of exon B to the start of exon 1 was suggested to include potential regulatory sites for the human HSL gene in adipocytes (Talmud 1998⁸³⁶, Grober 1997⁸³⁷) The region includes 15 N-boxes. Moreover, three pairs are located within short distances of each other measured in bp or helical turns. The pair at (+268, +272) (+279, +285) is separated by 5 bp or 1.0 HT. There are 6 bp the in the N-box and 5 bp distance between the N-boxes, or 11 bp from first nucleotide of the first N-box to first nucleotide of the second N-box. Since there are 10 base pair per helical turn, or 10 bp per HT, 11 bp is about 1.0 HT. The pair at (+936, +942) (+964, +970) is separated by 22 bp or 2.5 HT, and the pair at (+1,253, +1259) (+1270, +1276) is separated by 11 bp or 1.5 HT.

The 1.0, 2.5 and 1.5 helical turns separating the HSL N-boxes pairs are consistent with characteristic GABP heterotetramer binding. Moreover, the HSL testis-specific promoter also includes two N-boxes separated by 11 bp or 1.5 helical turns (Blaise 1999⁸³⁸). Many “TATA-less” promoters bind GABP through an N-box in their initiator element. Specifically, HSL is a TATA-less gene.

(b) Microcompetition

The effect of microcompetition on HSL transcription can be demonstrated by combining observations from two studies. Swiss mouse embryo; 3T3-L1 fibroblasts can be induced to differentiate into adipocyte-like cells. Undifferentiated cells contain very low level of HSL activity while differentiated adipocyte-like cells show much higher HSL activity (a 19-fold increase relative to undifferentiated cells) (Kawamura-1981⁸³⁹).

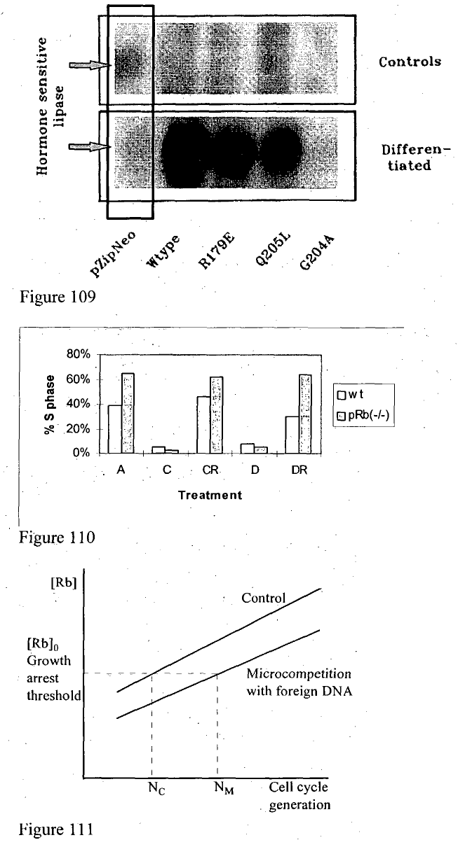

A study (Gordeladze 1997⁸⁴⁰) transfected 3T3-L1 preadipocytes with the pZipNeo vector, and then, induced the cells to differentiate by incubation with insulin (10 μg/ml), dexamethasone (10 nM), or iBuMeXan (0.5 mM), for 8 consecutive days following cell confluency. HSL mRNA was measured in differentiated 3T3-L1 cells and undifferentiated confluent controls. Although differentiated 3T3-L1 cells usually show significant HSL activity, cells transfected with pZipNeo showed less HSL mRNA than undifferentiated confluent controls (Gordeladze 1997, ibid, FIG. 11). Compare pZipNeo and Wtype columns in FIG. 109. The pZipNeo vector carries the Moloney murine leukemia virus LTR, which microcompeted with HSL promoter for GABP. Microcompetition between the viral LTR and HSL promoter decreases transcription of the HSL gene. Symbolically,
↑[N-boxᵥ]→↓[mRNA_{HSL}]
Sequence of quantative events 61: Predicted effect of foreign N-boxes on hormone sensitive lipase (HSL) mRNA levels.

(ii) HSL null mutants and adipocyte hypertrophy

A study (Osuga 2000⁸⁴¹) generated HSL knockout mice with homologous recombination in embryonic stem cells. Cholesterol ester hydrolase (NCEH) activities were completely absent from both brown adipose tissue (BAT) and white adipose tissue (WAT) in mice homozygous for the mutant HSL allele (HSL(−/−)). The cytoplasmic area of BAT and WAT adipocytes showed 5- and 2-fold increase, respectively, in HSL(−/−) mice compared to controls (Osuga 2000, ibid, FIGS. 3a and 3b), which indicates adipocyte hypertrophy. Denote adipocyte size with AdipoSize, then,
↓[mRNA_{HSL}]→↑AdipoSize
Sequence of quantative events 62: Predicted effect of hormone sensitive lipase (HSL) mRNA levels on adipocyte size (AdipoSize).

Note: The body weight of the HSL(−/−) mice was not different, at least until 24 weeks of age, from wild-type. The reason was probably a lack of adipocyte hyperplasia in HSL(−/−) mice. Consider the section on Rb gene below.

(iii) Logical Summary

According to the principle of transitive deduction:
If (↑[N-boxᵥ]→↓[mRNA_{HSL}]) AND (↓[mRNA_{HSL}]→↑AdipoSize) Then (↑[N-boxᵥ]→↑AdipoSize)

Since microcompetition with foreign N-boxes decreases HSL gene transcription, and since decreased HSL transcription increases adipocyte hypertrophy, microcompetition should increase adipocyte hypertrophy.

(iv) Decreased HSL mRNA in Obesity

A study (Large 1999⁸⁴²) measured HSL mRNA levels, protein expression, and enzyme activity in abdominal subcutaneous adipocytes from 34 obese drug-free and otherwise healthy males and females and 14 non-obese control subjects. The results showed decreased HSL mRNA, protein expression, and enzyme activity (Large 1999, ibid, Table 3). The findings were age and gender independent. Based on these results, Large, et al., (1999, ibid) concluded: “a decreased synthesis of the HSL protein at the transcriptional level is a likely factor behind the findings of decreased HSL expression in adipocytes from obese subjects. . . . Decreased HSL expression may at least in part explain the well-documented resistance to the lipolytic effect of catecholamines in obesity.” A subsequent study by the same laboratory also showed a 73% decrease in HSL protein levels in obesity (Elizalde 2000⁸⁴³, FIG. 4C, and Table 1).

An infection with a GABP virus decreases MT transcription, which increases body weight (see above). An infection with a GABP virus also decreases HSL transcription, which induces adipocyte hypertrophy (see above). Therefore, obesity, which results from an infection with a GABP virus, should show both weight gain and adipocyte hypertrophy. As expected, human obesity shows both symptoms (Garaulet 2002⁸⁴⁴).

(d) Retinoblastoma Susceptibility Gene (Rb)

(i) Rb is a Foreign N-box-Suppressed Gene

GABP stimulates Rb transcription (see chapter on cancer, p 358). Therefore, microcompetition with foreign N-boxes decreases Rb transcription. Symbolically,
↑[N-boxᵥ]→↓[mRNA_{Rb}]
Sequence of quantative events 63: Predicted effect of foreign N-boxes on retinoblastoma susceptibility gene (Rb) mRNA levels.

(ii) Rb Deficiency and Adipocyte Hyperplasia

A decrease in Rb expression decreases adipocyte differentiation and increases adipocyte proliferation. Consider the following observations. A study (Classon 2000⁸⁴⁵) measured the percentage of Rb-null (pRb(−/−)) 3T3 preadipocytes in S-phase in five different environments, cells grown in DMEM (asynchronous cells, marked A), cells grown to confluence in DMEM containing 10% calf serum and then maintained for 6 days in the same mixture (marked C), confluent cells split into subconfluent conditions (marked CR), confluent cells treated for 6 days with an adipocyte differentiating mixture (marked D), and differentiated cells split into subconfluent conditions (market DR). FIG. 110 presents the observations (Classon 2000, ibid, FIG. 3A).

Asynchronous pRb(−/−) cells showed a tendency for excessive cell replication. Moreover, pRb(−/−) differentiated cells showed higher probability for cell cycle re-entry. It should be emphasized that although pRb seems to affect the establishment of a permanent exit from cell cycle, pRb is not required. Expression of CCAT/enhancer binding protein α (C/EBPα) and peroxisome proliferator-activator receptor γ (PPARγ), bypassed the requirement for pRb, and caused pRb(−/−) cells to differentiate into adipocytes (Classon 2000, ibid, FIG. 1B).

Another study (Puigserver 1998⁸⁴⁶) tested the relation between pRb concentration and adipocyte differentiation by comparing proliferative and differentiated brown (primary) and white (3T3-F442A) adipocytes in culture. The differentiation was determined by detection of lipid accumulation and expression of the specific differentiation markers aP2 and UCP-1. The results showed almost undetectable pRb levels in proliferative undifferentiated cells. On the other hand, pRb was clearly detected in nuclei of differentiated primary brown adipocytes (Puigserver 1998, ibid, FIG. 2A), in 3T3-F442A cells with lipid accumulation in their cytoplasm and concomitant UCP-1 expression (Puigserver 1998, ibid, FIG. 3), and in 3T3-F442A cells with lipid accumulation and aP2 expression. Puigserver, et al., (1998, ibid) also note that “the pRb levels measured by immunoblotting clearly increased during differentiation of 3T3 F442A cells (Puigserver 1998, ibid, FIG. 2B),” and that “there was an apparent positive correlation between pRb expression and lipid accumulation, since nuclei from cells with more lipid droplets in their cytoplasm were more strongly immunostained for pRb than those of cells with less lipid droplets (Puigserver 1998, ibid, FIG. 2A).”

A study (Richon 1992⁸⁴⁷) proposed a model for the relation between cell differentiation and hyperplasia. A signal increases Rb transcription, which increases the concentration of hypophosphorylated and total-pRb. The increase in hypo-pRb prolongs G1. However, the initial increase in hypo-pRb, most likely, is insufficient for permanent G1 arrest. Therefore, cells reenter the cell cycle for a few more generations. While cells continue to divide, the increased rate of transcription increases the concentration of hypo-pRb. When a critical hypo-pRb concentration, or threshold, is reached, the cells irreversibly commit to terminal differentiation. The model describes the determination of the commitment to differentiate as a stochastic process with progressive increases in the probability of both G1/G0 arrest and differentiation established through successive cell divisions. Such model predicts an increase in the number of cell cycle generations required for the production of the threshold Rb concentration, under conditions of suppressed Rb transcription. Consider FIG. 111.

Microcompetition decreases Rb transcription. Therefore, the number of generations required to reach the required Rb concentration ([Rb]₀) under microcompetition (N_{M}) is greater than the number in controls (N_{C}). In obesity, therefore, one should observe excessive replication in vitro (Roncari 1986⁸⁴⁸, Roncari 1981⁸⁴⁹), and hyperplasia in vivo. Denote adipocyte number with [Adipocytes], then,
↓[mRNA_{Rb}]→↑[Adipocytes]
Sequence of quantative events 64: Predicted effect of Rb mRNA on number of adipocytes.

(iii) Logical Summary

According to the principle of transitive deduction:
If (↑[N-boxᵥ]→↓[mRNA_{Rb}]) AND (↓[mRNA_{Rb}]→↑[Adipocytes]) Then (↑[N-boxᵥ]→↑[Adipocytes])

Since microcompetition with foreign N-boxes decreases Rb gene transcription, and since decreased Rb transcription increases adipocyte hyperplasia, microcompetition should increase adipocyte hyperplasia.

Consider the non-obese HSL(−/−) mice (Osuga 2000, ibid, see above). Both HSL and Rb are foreign N-box-suppressed genes. Therefore, both genes should show decreased expression in obesity, resulting in adipocyte hypertrophy and hyperplasia. Since Rb transcription is most likely independent of HSL expression, Rb in HSL(−/−) mice is not under-expressed, and the adipocytes in HSL(−/−) mice are not hyperplastic.

(2) Infection with GABP Viruses and Obesity

(a) Human Adenovirus 36 (Ad-36)

A recent study (Dhurandhar 2000⁸⁵⁰) inoculated chickens and mice with the human adenovirus Ad-36. Weight matched groups were inoculated with tissue culture media as controls. The Ad-36 inoculated and uninfected control animals were housed in separate rooms under bio-safety level 2 or better containment. The chicken study was repeated three times. The first chicken experiment included an additional weight matched group of chickens that was inoculated with CELO (chick embryo lethal orphan virus), an avian adenovirus. Food intake and body weight were measured weekly. At the time of sacrifice, blood was drawn, and visceral fat was separated and weighed. Total body fat was determined by chemical extraction of carcass fat. The results of experiments 1, 2, and 3 showed an increase of 100%, 128%, and 74% in visceral fat of Ad-36 chickens compared to controls, respectively (Dhurandhar 2000, ibid, Table 1, 3 and 4). All three experiments showed no difference in food intake and body weight between Ad-36 chickens and controls. Chickens inoculated with CELO virus showed no change in visceral fat. In Ad-36 mice, visceral fat was 67% greater than controls, and mean body weight was 9% greater. There was no difference in food intake, and sections of the brain and hypothalamus of Ad-36 inoculated animalsshowed no overt histopathological changes. Ad-36 DNA could be detected in adipose tissue but not skeletal muscles of randomly selected animals for as long as 16 weeks after inoculation. Based on these results, Dhurandhar (2000, ibid) concluded: “the role of viral disease in the etiology of human obesity must be considered.”

(b) HIV

Recently, several studies documented a new syndrome associated with HIV infection termed “lipodystrophy,” or “fat redistribution syndrome” (FRS). The typical symptoms of FRS, such as peripheral lipodystrophy, central adiposity, hyperlipidemia and insulin resistance (for a recent review see Behrens 2000⁸⁵¹), are similar to symptoms associated with syndrome X (Engelson 1999⁸⁵²) (Syndrome X is also known as “insulin resistance,” or plain “obesity.”) The cause of FRS is unknown. The temporal association between the discovery of FRS and the adoption of the protease inhibitor, medications has led several investigators to conclude that FRS is a result of the therapy. However, since FRS was also identified in HIV-infected patients on alternative therapies, other researchers concluded that FRS might be a characteristic of the HIV infection, only unmasked by prolonged survival associated with protease inhibitors treatment.

HIV is a GABP virus. HIV infection results in microcompetition between the viral DNA and host genes, which, in turn, leads to obesity.

Note: Recent studies reported that HIV infection is associated with a greater risk of atherosclerosis, stroke, and insulin resistance (Hui 2003⁸⁵³, Rabinstein 2003⁸⁵⁴, Beregszaszi 2003⁸⁵⁵, Madamanchi 2002⁸⁵⁶, Seminari 2002⁸⁵⁷, Depairon 2001⁸⁵⁸). Atherosclerosis, stroke, and insulin resistance are also result from microcompetition with foreign N-boxes (see chapters on atherosclerosis, p 161, stroke, p 271, and signal resistance, p 340). Therefore, the clustering of obesity, atherosclerosis, stroke, and insulin resistance in the same patients is consistent with the predicted effect of an infection with a GABP virus.

(3) Viral N-box Copy Number and Weight-Gain

(a) General Prediction

An exogenous event that increases [N-boxᵥ] should increase body weight. Symbolically, [symbol]
Exogenous event→↑[N-boxᵥ]→↑Body weight
Sequence of quantative events 65: Predicted effect of an exogenous event that increases viral N-box copy number on body weight.

(b) Observations

(i) Transplantation

Numerous studies showed an increase in genome copy number of cytomegalovirus (CMV), or Epstein-Barr virus (EBV) following transplantation, resulting from a primary CMV infection in seronegative hosts, or reactivation of a persistent infection in seropositive hosts (see for instance, Norris 2002⁸⁵⁹, Kogan-Liberman 2001⁸⁶⁶, Rao 2000⁸⁶¹). In many cases, the increase in viral load is associated with the immunosuppression treatment administered to the transplant recipients.

Cytomegalovirus, and Epstein-Barr virus are GABP viruses. According to the general prediction above, transplantation should be associated with weight gain. Symbolically, [symbol]
Transplantation→↑[N-boxᵥ]→↑Body weight
Sequence of quantative events 66: Predicted effect of transplantation on body weight.

As expected, numerous studies showed a weight-gain following transplantation (Baum 2002⁸⁶², Richardson 2001⁸⁶³, Clunk 2001⁸⁶⁴, van den Ham 2000⁸⁶⁵, Mor 1995⁸⁶⁶, Johnson 1993⁸⁶⁷, Palmer 1991⁸⁶⁸).

Note: In addition, as expected, transplantation is associated with increase susceptibility to cardiovascular disease (Baum 2002, ibid) (see chapter on atherosclerosis, p 161).

(ii) Chemotherapy

The CMF adjuvant combination includes cyclophosphamide, methotrexate, and 5-fluorouracil. CMF is currently the recommended chemotherapy for pre-menopausal women with stage II, or poor prognosis stage I breast cancer, and for post-menopausal women with similar disease characteristics.

Several studies reported observations consistent with an increase in genome copy number of CMV following treatment with the immunosuppression agent cyclophosphamide (Tebourbi 2002⁸⁶⁹, Palmon 2000⁸⁷⁰, Qamruddin 2001⁸⁷¹, Schmader 1992⁸⁷², Price 1991⁸⁷³, Smee 1991⁸⁷⁴, Bale 1991⁸⁷⁵).

CMV is a GABP virus. According to the general prediction above, treatment with cyclophosphamide should be associated with weight gain. Symbolically, [symbol]
[Cyclophosphamide]→↑[N-boxᵥ]→↑Body weight
Sequence of quantative events 67: Predicted effect of Cyclophosphamide on body ewight.

As expected, several studies showed an increase in body weight in breast cancer patients following treatment with CMF (Del Rio 2002⁸⁷⁶, Lankester 2002⁸⁷⁷, Aslani 1999⁸⁷⁸, Sitzia 1998⁸⁷⁹).

Notes:

1. Demark-Wahnefried 1997⁸⁸⁰ showed a decrease in resting metabolic rate (RMR) following treatment with CMF.

2. Kutynec 1999⁸⁸¹ showed a tendency for weight gain and increase in percent body fat following treatment with adjuvant chemotherapy using Adriamycin and cyclophosphamide (AC).

(4) Obesity and Other Chronic Diseases

Conditional probability is the probability of an event given that another event has occurred. Consider two diseases, denoted Diseaseᵢ and Diseaseⱼ, respectively. Denote the conditional probability of Diseaseⱼ given Diseaseᵢ with p(Diseaseⱼ|Diseaseᵢ). If p(Diseaseⱼ|Diseaseᵢ)>p(Diseaseⱼ), that is, the probability of Diseaseⱼ is greater in individuals with Diseases compared to the general population, then Diseaseⱼ and Diseaseᵢ will be called positively dependent, or for short, dependent.

Condition 1: Cross-Tissue Transmission

Assume an infection with a GABP virus of a certain cell type, denoted Cellᵢ, results in Diseaseᵢ. Also, assume that an infection with the same GABP virus, of another cell type, denoted Cellⱼ, results in Diseaseⱼ. If the probability of viral transmission from Cellᵢ to Cellⱼ, in the same individual, is greater than zero, then Diseaseⱼ and Diseaseᵢ will show characteristics of dependent diseases. Condition 1 will be called “cross-tissue transmission.”

Condition 2: Cross-Viral Immunosuppression

Assume an infection with a certain GABP virus, denoted Vᵢ, of a certain cell type, denoted Cellᵢ, results in Diseaseᵢ. Also, assume that an infection with another GABP virus, Vⱼ, of another cell type, denoted Cellⱼ, results in Diseaseⱼ. If an infection with Vᵢ is associated with an increase in probability of an infection with Vⱼ, then Diseaseⱼ and Diseaseᵢ will show characteristics of dependent diseases. Condition 2 will be called “cross-viral immunosuppression.”

Notes:

1. A decrease in efficiency of the cell-mediated immunity increases the probability of both types of infections. In such a case, an infection with Vᵢ will be associated with an increase in probability of an infection with Vⱼ.

2. If infection with Vᵢ directly increases susceptibility to a Vⱼ infection, an infection with Vi will be associated with increase in probability of an infection with Vⱼ.

Several studies reported that obese patients show an increased risk for other chronic diseases, such as, cardiovascular disease (Dubbert 2002⁸⁸², Wilson P W 2002⁸⁸³, Jousilahti 1996⁸⁸⁴, Licata 1993⁸⁸⁵, Kannel 1991⁸⁸⁶, Hubert 1983⁸⁸⁷, Gordon 1976⁸⁸⁸, Kannel 1967⁸⁸⁹), cancer (Bianchini 2002⁸⁹⁰, Bergstrom 2001⁸⁹¹, McTiernan 2000⁸⁹², Guthrie 1999⁸⁹³, Carroll 1998⁸⁹⁴), and osteoarthritis (see chapter on osteoarthritis, p 351).

Cross-tissue transmission, and cross-viral immunosuppression can explain the observed relations between obesity and the other chronic diseases.

(5) The Obesity Epidemic

An increase in ultraviolet B (UV-B) radiation decreases the effectiveness of the cell-mediated immunity (Kasahara 2002⁸⁹⁵, Kasahara 2001⁸⁹⁶, Garssen 1999⁸⁹⁷), which increases susceptibility to infections. For instance, several studies showed viral reactivation from latency following exposure to UV-B (Keadle 2002⁸⁹⁸, El-Ghorr 1999⁸⁹⁹, Walker 1998⁹⁰⁰, Blatt 1993⁹⁰¹, Miller 1993⁹⁰², Rooney 1992⁹⁰³, Laycock 1991⁹⁰⁴). See also recent reviews Clydesdale 2001⁹⁰⁵ and Garssen 2001⁹⁰⁶ Consider the following sequence of quantitative events. [symbol]
[UB-B]→↑[N-boxᵥ]→↑[Obesity]
Sequence of quantative events 68: Predicted effect of ultraviolet B on obesity.

The recent environmental increase in UV-B radiation, can, therefore, be one of the exogenous events responsible for the recent observed increase in obesity, type II diabetes Kaufman 2002⁹⁰⁷, Seidell 2000⁹⁰⁸, Rosenbloom 1999⁹⁰⁹, Jovanovic 1999⁹¹⁰, cardiovascular disease Deedwania 2003⁹¹¹, Bonow 2002⁹¹², Reddy 1998⁹¹³, certain types of cancer, such as esophageal adenocarcinoma (el-Serag 2002⁹¹¹), nonmelanoma skin cancer (Limmer 2001⁹¹⁵); melanoma (Dennis 1999⁹¹⁶), and non-Hodgkin's lymphoma (Weisenburger 1994⁹¹⁷), and autoimmune diseases, such as asthma (Kheradmand 2002⁹¹⁸, Umetsu 2002⁹¹⁹, Holgate 1999⁹²⁰), and type I diabetes (Silink 2002⁹²¹, Kida 2000⁹²²).

Notes:

1. UV-B radiation and breast-feeding have opposite effects on [N-boxᵥ], see chapter on treatment, p 444.

2. A recent series of articles by U. N. Das suggested that obesity might be an inflammatory condition (see, for instance, Das 2002A⁹²³, Das 2002B⁹²⁴, and Das 2001⁹²⁵). Since the cause of obesity is a latent infection with a GABP virus, obesity can be viewed as an inflammatory condition.

c) Other Disruptions in p300 Allocation

(1) Prediction

Let G denote a GABP regulated gene and “v” a GABP virus. The following function summarizes the effect of microcompetition, GABP kinases, and redox on GABP transcription (the function is called the “allocation model” of transcription, see chapter on signaling and allocation, p 330).

$$\begin{matrix}[ {mRNA}_{G} ] = & {f}_{A - T} ( [ {DNA}_{G - GABP} ] , & [ {DNA}_{v - GABP} ] , & {Affinity}_{v / G} , & [ {GABPkinase}^{phos} ] , & OS ) \\ GABP & ( + ) & ( - ) & ( - ) & ( + ) & ( - ) \\ stimulated gene & & & & & \end{matrix} \qquad Function 32$$

According to the “allocation model” of transcription, microcompetition between viral and cellular N-boxes for p300·GABP, excessive decrease in phosphorylation of a GABP kinase, and excessive increase in oxidative stress, decreases transcription of a GABP stimulated gene.

Let AGENT be a GABP kinase agent. Consider an exogenous event that decreases the concentration of AGENT. In steady state, such event decreases transcription of relevant GABP regulated genes. Symbolically, [symbol]
[AGENT]→↓[GABPkinase^{phos}]→↓[p300·GABP·N-box_{c}]→↓[mRNA_{G}]
Sequence of quantative events 69: Predicted effect of a GABP kinase agent on mRNA of a GABP regulated gene.

The section above on transitive deduction identified several GABP stimulated genes: HMT-II_{A}, HSL, and Rb. Decreased transcription of these genes resulted in symptoms of obesity. Symbolically,
↓[mRNA_{G}]→↑[Obesity], for g—hMT-H_{A}, HSL, or Rb
Sequence of quantative events 70: Predicted effect hMT-H_{A}, HSL, or Rb mRNA on obesity.

According to the principle of transitive deduction:
If (↓[AGENT]→↓[mRNA_{G}]) AND (↓[mRNA_{G}]→↑[Obesity]) Then (↓[AGENT]→↑[Obesity])

An exogenous event, which decreases transcription of these genes, should result in obesity.

The following section tests the prediction with various exogenous events, such as genetic mutation, injury, and diet.

(2) Observations

(a) Leptin

Leptin is an ERK agent. A study (Yamashita 1998⁹²⁶) showed increased tyrosine phosphorylation of STAT3 and ERK in Chinese hamster ovary (CHO) cells following binding of leptin to the long form leptin receptor. CHO cells with a mutated leptin receptor showed diminished phosphorylation. According to the prediction, a mutation in leptin, or the long form leptin receptor, which decreases the intensity of the leptin-mediated signal should result in obesity.

As expected, certain homozygous mutations in leptin, or leptin receptor, lead to early-onset obesity and hyperphagia (Clement 1998⁹²⁷). Mutation in the ob (leptin) gene is associated with obesity in the ob/ob mouse. Obesity in the db/db mouse is associated with mutations in the db (leptin receptor) gene. An alternatively spliced transcript of the leptin receptor encodes a form with a long intracellular domain. The db/db mouse produces the alternatively spliced transcript with a 106-nucleotide insertion that prematurely terminates the intracellular domain. The db/db mouse also exhibits a point mutation (G→T) in the same gene. The long intracellular domain form of the receptor participates in signal transduction, and the inability to produce the long form in db/db mice contributes to their extreme obese phenotype (Chen H 1996⁹²⁸). Obesity in the Zucker fatty (fa/fa) rats is associated with mutations in the fa gene, which also encodes a leptin receptor. The fa missense mutation (269 gln→pro) in the extracellular domain of the leptin receptor decreases cell-surface expression, leptin binding affinity, and signaling to the JAK-STAT pathway (da Silva 1998⁹²⁹).

(b) Estradiol

Estradiol is an ERK agent (see references in the chapter on signal resistance, p 340). The ovaries in polycystic ovary syndrome (PCOS) produce less estradiol in response to follicle-stimulating hormone (Caruso 1993⁹³⁰). According to the prediction, PCOS should be associated with obesity, insulin resistance, and hyperinsulinemia (see chapter on signal resistance, p 340).

As expected, PCOS is associated with high blood pressure, insulin resistance, hyperinsulinemia, and obesity. Ovariectomy also decreases the concentration of estradiol, sometimes to undetectable levels (Wronski 1987⁹³¹). As expected, ovariectomy is associated with obesity.

(c) Metallothionein (MT)

MT is a receptor of the ERK agent zinc. According to the prediction, a genetic deficiency in MT expression should result in obesity.

As expected, MT-null mice are obese (Beattie 1998, ibid, see above).

(d) CD18

A study (Flaherty 1997⁹³²) engineered Chinese hamster ovary (CHO) fibroblast cell lines to express the CD11a/CD18 or CD11b/CD18 integrin antigens. Upon heterologous expression of CD11a/CD18 and CD11b/CD18, the otherwise non-responsive fibroblasts became responsive to the ERK agent LPS. Another study (Ingalls 1995⁹³³) also showed cell activation following binding of LPS to CD11c/CD18. A follow-up study (Ingalls 1997⁹³⁴) showed transmission of a signal following binding of LPS to both wild-type CD11b/CD18 and mutant CD11b/CD18 lacking the cytoplasmic domain. These studies indicate that CD11a/CD18 and CD11b/CD18 are receptors for the ERK agent LPS. According to the prediction, a mutation in CD11a/CD18, CD11b/CD18, or their receptors, which decreases the intensity of the integrin-mediated signal, should result in obesity.

Note: Although full length CD11b/CD18 is needed for productive phagocytic signals, LPS-mediated activation does not require the cytoplasmic domains. Perhaps CD11b/CD18 activates cells by presenting LPS to a downstream signal transducer (Ingalls 1997, ibid).

CD11a/CD18 binds ICAM-1 and MAC-1. As expected, ICAM-1, or MAC-1 null mice, are obese (Dong 1997⁹³⁵).

(e) Zinc and Copper

Zinc and copper are ERK agents. According to the prediction, low intake of zinc should result in obesity.

As expected, several studies showed correlation between body weight and low zinc intake, or low zinc concentrations in plasma (Ledikwe 2003⁹³⁶, Ozata 2002⁹³⁷, Marreiro Ddo 2002⁹³⁸)

Note: A study (Singh R B 1998⁹³⁹), which surveyed 3,575 subjects, aged 25 to 64 years, also showed, as expected, a correlation between the prevalence of coronary artery disease (CAD), diabetes, and glucose intolerance and lower intake of dietary zinc.

(3) Summary

An exogenous event, or disruption, which results in an excessive decrease in GABP kinase phosphorylation, is associated with obesity.

d) Complements

(1) Model

Let A and B be two GABP kinase agents with corresponding pathways (A, GABP) and (B, GABP). If A is not a GABP kinase receptor for B, that is, A does not belong to the (B, GABP) pathway, B will be called a “complement” for A. Notice that the relation is asymmetric. In the (A, B, GABP) pathway, B is a complement for A, but A is not a complement for B.

If B is a complement for A, administration of B can alleviate symptoms associated with a deficiency in A, or an A receptor. The prediction will be called the “complement prediction.”

(2) Observations

Consider leptin with the corresponding (leptin, leptin receptor, GABP) pathway. A mutation in leptin or the leptin receptor results in obesity (see above).

(a). Leptin and IL-1β

Consider the ERK agent IL-1β. Assume that leptin is not in the (IL-1β, GABP) pathway. According to the complement prediction, administration of IL-1β to leptin or leptin receptor mutated animals should diminish the intensity of obesity-associated symptoms, such as increased body weight, or insulin resistance, in these animals.

As expected, a study (Ilyin 1996⁹⁴⁰) showed a 66.1% decrease in nighttime food intake following chronic intracerebroventricular (ICV) microinjection of IL-1β to obese (fa/fa) Zucker rats. Another study (del Rey 1989⁹⁴¹) showed normalization of glucose blood levels for several hours following a low dose injection of human recombinant IL-1β to genetically obese ob/ob or db/db mice.

Note: Luheshi 1999⁹⁴² showed that IL-1β is an ERK receptor for leptin, that is, leptin belong to the following pathway: (Leptin, IL-1β, GABP). However, IL-1β can still be a complement for leptin if leptin is not a receptor for IL-1β, that is, leptin does not belong to the following pathway: (IL-1β, GABP) (see the asymmetry of the complement condition described above).

(b) Leptin and TNFα

Consider the ERK agent TNFα. Assume that leptin in not in the (TNFα, GABP) pathway. According to the complement prediction, administration of TNFα to leptin or leptin receptor mutated animals should diminish the intensity of obesity-associated symptoms in these animals.

As predicted, ICV microinjection of TNFα (50, 100 and 500 ng/rat) to obese (fa/fa) Zucker rats in triplicate decreased short-term feeding (4 hours) by 17%, 20%, and 20%, nighttime feeding (12 hours) by 13%, 14% and 13%, and total daily food intake by 11%, 12% and 11%, respectively (Plata-Salaman 1997⁹⁴³).

(c) Leptin and LPS

Consider the ERK agent LPS. Assume that leptin in not in the (LPS, GABP) pathway. According to the complement prediction, administration of LPS to leptin or leptin receptor mutated animals should diminish the intensity of obesity-associated symptoms in these animals.

As predicted, administration of LPS (0.1, 1, 10, 100 μg) to db/db, leptin receptor deficient mice, induced a significant decrease in food intake (25%, 40%, 60%, 85%, respectively, in the first 24 hours post injection). The effect on leptin deficient ob/ob mice was similar (Faggioni 1997⁹⁴⁴).

e) Summary

The microcompetition model of obesity explains many previously unexplained observations reported in the obesity literature. The observations include decreased expression of hormone sensitive lipase, hypertrophy and hyperplasia of adipocytes, catecholamine resistance, the excessive need for oxytocin stimulation of labor and decreased lactational performance in obese mothers, insulin resistance, leptin resistance, hyperinsulinemia, hyperleptinemia, the high level of serum zinc and copper, and the high level of serum estradiol in obesity, and the effectiveness of IL-1β, TNFα and LPS in attenuating symptoms of obesity in ob/ob and db/db mice and fa/fa rats. Moreover, the model proposes a new conjecture on the cause of the obesity epidemic (for some explanations see the chapter on signal resistance, p 340).

9. Technical Note: Signaling and Allocation

a) Signaling

(1) Conceptual Building Blocks

(a) ERK Pathway

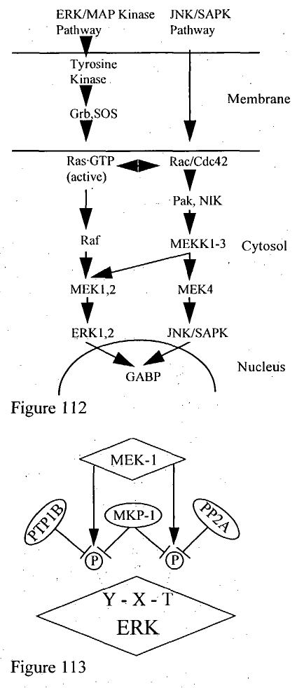

The signal associated with the extracellular signal-regulated kinase (ERK, also called mitogen activated protein (MAP) kinase) cascade propagates through sequential activation of multiple kinases (see left side in FIG. 112). An important kinase in the ERK cascade is Raf, which phosphorylates MEK, which, in turn, phosphorylates ERK. Raf, also called MAPKKK, is activated by a yet unknown mechanism usually dependent upon Ras. Following interaction with Ras, Raf translocates to the plasma membrane, an apparently important step for activation. Other kinases can also function in the capacity (i.e.—MEKKs 1 and 3). Raf activates the MAPKK MEK (MEK1 and MEK2), which activates ERK by dual phosphorylation on a Thr-Xaa-Tyr motif after which ERK translocates to the nucleus and functions as proline-directed Ser/Thr kinase of transcription factors with minimal target sequence of Ser/Thr-Pro (Hipskind 1998⁹⁴⁵).

Dephosphorylation of Thy or Tyr inactivates ERK. FIG. 113 illustrates activation of ERK by MEK-1, a MAPKK, and deactivation by PP2A, a serine/threonine phosphatase, PTP1 B, a tyrosine specific phosphatase, or MKP-1, a dual specificity phosphatase. A diamond represents a kinase, an ellipse, a phosphatase, an arrow, phosphorylation, and a T-headed line, dephosphorylation.

(b) ERK Agent

A molecule, which stimulates phosphorylation of ERK, will be called an ERK agent. Let [ERK agent] denote the concentration of an ERK agent and [ERK^{phos}] the concentration of phosphorylated ERK. The following function presented the effect of an ERK agent on ERK phosphorylation.

$$\begin{matrix}[ {ERK}^{phos} ] = & f ( [ ERK agent ] ) \\ & ( + )\end{matrix} \qquad Function 33$$

Examples of ERK agents include sodium butyrate (SB), trichostatin A (TSA), trapoxin (for SB, TSA and trapoxin see in Espinos 1999⁹⁴⁶), phorbol ester (phorbol 12-myristate 13-acetate, PMA, TPA), thapsigargin (for PMA and thapsigargin see Shiraishi 2000⁹⁴⁷, for PMA see Herrera 1998⁹⁴⁸, Stadheim 1998⁹⁴⁹), retinoic acid (RA, vitamin A) (Yen 1999⁹⁵⁰), interferon-γ (IFNγ) (Liu 1994⁹⁵¹, Nishiya 1997⁹⁵²), heregulin (HRG, new differentiation factor, NDF, neuregulin, NRG) (Lessor 1998⁹⁵³, Marte 1995⁹⁵⁴, Sepp-Lorenzino 1996⁹⁵⁵, Fiddes 1998⁹⁵⁶), zinc (Zn) (Park J A 1999⁹⁵⁷, Kiss 1997⁹⁵⁸), copper (Cu) (Wu 1999⁹⁵⁹, Samet 1998⁹⁶⁰, both studies also show phosphorylation of ERK1/2 by Zn), estron, estradiol (Migliaccio 1996⁹⁶¹, Ruzycky 1996⁹⁶², Nuedling 1999⁹⁶³), interleukin 1β (IL-1β) (Laporte 1999⁹⁶⁴, Larsen 1998⁹⁶⁵), interleukin 6 (IL-6) (Daeipour 1993⁹⁶⁶), tumor necrosis factor α (TNFα) (Leonard 1999⁹⁶⁷), transforming growth factor β (TGFβ) (Hartsough 1995⁹⁶⁸, Yonekura 1999⁹⁶⁹, oxytocin (OT) (Strakova 1998⁹⁷⁰, Copland 1999⁹⁷¹, Hoare 1999, ibid). All studies show phosphorylation of ERK1/2 by these agents.

(2) Model: ERK Phosphorylation of GABP

ERK phosphorylates GABPα and GABPβ. However, phosphorylation does not change the capacity of GABP to bind DNA (Flory, 1996, ibid, Avots, 1997, ibid, Hoffmeyer, 1998⁹⁷², Tomaras 1999⁹⁷³). Phosphorylation is known to increase the binding of p300 to other transcription factors, such as NF-κB unit p65 and Bbf, or to stabilize their complexes (Zhong 1998⁹⁷⁴, Bevilacqua 1997⁹⁷⁵). The following function presents a similar role for ERK phosphorylation of GABP (referred to as the “ERK phosphorylation of GABP” model). [p300·GABP] denotes concentration of p300·GABP.

$$\begin{matrix}[ p300 \cdot GABP ] = & {f}_{ERK} ( [ {ERK}^{phos} ] ) \\ & ( + )\end{matrix} \qquad Function 34$$

An increase in concentration of phosphorylated (active) ERK increases concentration of phosphorylated GABP, which, in turn, increases binding of p300 to GABP or stabilizes the p300·GABP complex, which increases the concentration of p300·GABP. Symbolically,
↑[ERK^{phos}]→↑[GABP^{phos}]→↑[p300·GABP]
Sequence of quantative events 71: Predicted effect of ERK phosphorylation on the p300·GABP complex.

Consider a GABP stimulated gene G, then,
↑[ERK^{phos}]→↑[GABP^{phos}]→↑[p300·GABP·N-box_{G}]→↑[mRNA_{G}]
Sequence of quantative events 72: Predicted effect of ERK phosphorylation on transcription of a GABP stimulated gene.

(3) Prediction

Many studies, reported the concentration of ERK agents instead of the concentration of phosphorylated ERK. To accommodate the variation, the [ERK^{phos}]=f([ERK agent]) relation (above) can be used to extend the sequence of quantitative events.
↑[ERK agent]→↑[ERK^{phos}]→↑[GABP^{phos}]→↑[p300·GABP·N-box_{G}]→↑[mRNA_{G}]
Sequence of quantative events 73: Predicted effect of an ERK agent on transcription of a GABP stimulated gene.

According to the sequence of quantitative events, an increase in the concentration of an ERK agent should increase transcription of a GABP stimulated gene. Consider the following observations.

(4) Observations

The following observations related to different segments of the above sequence of quantitative events.

(a) N-box DNase-I Hypersensitivity

Histone acetylation occurs post-translationally, and reversibly, on the ε-NH₃+ groups of lysine residues embedded in the N-terminal tails of the core histones. The reaction is catalyzed by histone acetyltransferases (HATs), which transfer the acetyl moiety from acetyl coenzyme A. Introduction of the acetyl group to lysine neutralizes the positive charge, increases hydrophobicity, and leads to the unfolding of chromatin (Kuo 1998⁹⁷⁶). Histone hyperacetylation correlates with sensitivity to digestion by deoxyribonuclease I (DNase-I) (Hebbes 1994⁹⁷⁷). Moreover, binding of a transcription complex with HAT activity to DNA enhances DNase-I hypersensitivity around the DNA binding site. p300 has HAT enzymatic activity, therefore, p300·GABP binding to DNA enhances DNase-I hypersensitivity around the N-box.

The major transcription factor that binds the enhancer site in the third intron of TNFα gene is GABP (Tomaras 1999, ibid). Porcine peripheral blood mononuclear cells (PBMC) were stimulated with the ERK agent TPA and the DNase-I hypersensitivity of the third intron enhancer of the TNFα gene was measured.

Denote the increase in the DNase-I hypersensitivity of the third intron enhancer of TNFα with [DNase-I_{3rd intron}] According to the “ERK phosphorylation of GABP” model, [symbol]
[TPA]→↑[ERK^{phos}]→↑[GABP^{phos}]→↑[p300·GABP·N-box_{3rd intron}]→↑[DNase-I_{3rd intron}]
Sequence of quantitative events 74: Predicted effect of TPA on DNase-I hypersensitivity of the third intron enhancer of TNFα.

Treatment of PBMC with TPA should increase DNase-I hypersensitivity of the third intron enhancer of TNFα.

As expected, the results showed that TPA consistently enhanced DNase-I hypersensitivity of the third intron enhancer of the TNFα gene (Kuhnert 1992⁹⁷⁸). TPA treatment phosphorylated ERK, which phosphorylated GABP. Phosphorylation of GABP increased its binding to p300. The HAT activity of p300 acetylated the histones in the N-box binding site and enhanced DNase-I hypersensitivity of the third intron enhancer.

(b) Synergy with GABP Stimulation

Activated Raf-1 kinase phosphorylates and activates MAP kinase kinase (i.e. MEK), which phosphorylates and activates ERK. Since ERK phosphorylates GABP, the sequence of quantitative events can be summarized as follows, [symbol]
[Raf-1]→↑[MEK]→↑[ERK^{phos}]→↑[GABP^{phos}]
Sequence of quantitative events 75: Predicted effect of Raf-1 on GABP phosphorylation.

A study confirmed the sequence of quantitative events by showing that GABP is phosphorylated in vivo by Raf-1 kinase activators (e.g. serum and TPA) as well as constitutive versions of Raf-1 kinase (Flory 1996, ibid).

The same study cotransfected NIH 3T3 cells with an HIV LTR reporter construct (L3BCAT), either with constitutively active raf-1 (Raf-BXB), or inactive Raf-1 (Raf-BXB-301), and/or GABPα and/or β expression vectors. According to the “ERK phosphorylation of GABP” model, [symbol]
[Raf-BXB]→↑[MEK]→↑[ERK^{phos}]→↑[GABP^{phos}]→↑ [symbol] p300·GABP ·N-Box_{HIV LTR}]→↑↑↑[mRNA_{CAT}]
Sequence of quantitative events 76: Predicted effect of a constitutively active raf-1 and p300 on transcription of an HIV LTR reporter construct.

The two boxed upward arrows indicate the two exogenous events. The three upward arrows indicate a “more than additive” effect, see Herschlag 1993⁹⁷⁹ for discussion on additivity.

The results showed a 9- and 3-fold increase in CAT reporter gene expression in cells transfected with Raf-BXB without GABP, and cells transfected with GABPα, GABPβ, and Raf-BXB-301, the Inactive Raf-1, respectively. In contrast, cells transfected with Raf-BXB, the active Raf-1, and GABPα, GABPβ showed a 33-fold increase in reporter gene expression (Flory 1996, ibid, FIG. 2B). The “more than additive” effect (i.e. 9-fold+3-fold<33-fold) is consistent with the proposed model.

(c) Inhibition of p300 Binding

GABP binds a region of p300 between amino acids 1572 and 2370 (Bannert 1999, ibid), while the adenovirus E1A protein binds p300 between amino acids 1572 and 1818 (Eckner 1994⁹⁸⁰). Due to the binding site overlap, E1A displaces GABP from p300. According to the “ERK phosphorylation of GABP” model, such displacement should decrease the effectiveness of GABP phosphorylation. In support of the notion, activation of the SV40 minimal promoter by the ERK agent sodium butyrate and by p300 was suppressed by the adenovirus E1A protein (Espinos 1999, ibid).

(d) N-box Mutation

The human IL-2 gene contains a transcription enhancer (−502, −413) that binds GABP through two N-boxes. Cotransfection of a CAT reporter gene, controlled by multiple copies of the enhancer with GABPα and GABPβ expression vectors, into Jurkat cells and A3.01 T-cells increased CAT activity (Avots 1997, ibid, FIG. 7A). Moreover, mutations within the N-boxes abolished any induction. Cotransfection of a CAT reporter gene controlled by the IL-2 promoter-enhancer region (−583, +5) together with Raf-BXB, a constitutively-active version of c-Raf, and GABPα, GABPβ, resulted in a 3.5-fold increase in CAT activity (Avots 1997, ibid, FIG. 7A). According to the “ERK phosphorylation of GABP” model, mutation of one of the N-boxes should decrease the Raf-BXB+GABP effect on CAT expression. Symbolically, [symbol]
[Raf-BXB]→↑[ERK^{phos}]→↑[GABP^{phos}]→↑[ [symbol] p300·GABP· [symbol] N-Box_{IL-2}]→↓↑[mRNA_{CAT}]
Sequence of quantitative events 77: Predicted effect of a constitutively active raf-1, p300, and a mutation in one of the IL-2 N-boxes on transcription of CAT reporter gene controlled by the (−583, +5) promoter-enhancer region of IL-2.

The first boxed arrow indicates over expression of Raf-BXB, the second boxed arrow, over expression of GABP, the third, mutation in the N-box.

The results showed that cotransfection of Raf-BXB and GABP with a mutated N-box resulted in a 2-fold increase in CAT activity (Avots 1997, ibid, FIG. 7B). The impaired increase in CAT activity (2-fold<3.5-fold) is consistent with the proposed model.

(5) Conclusions

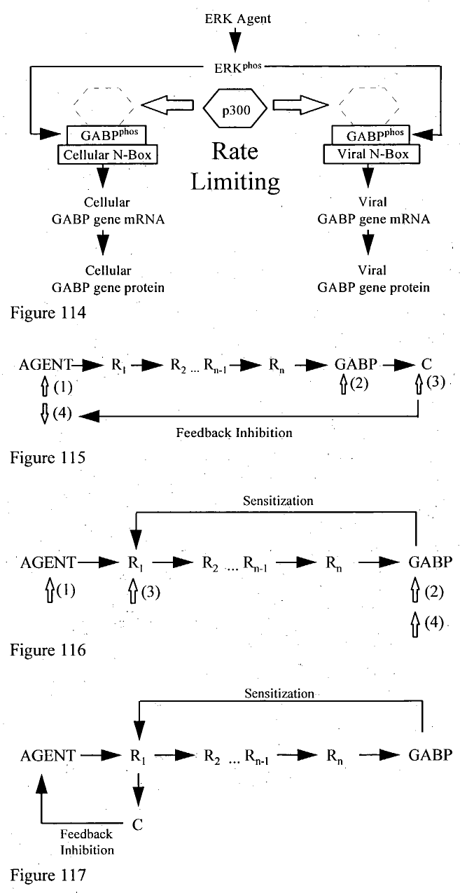

Microcompetition between a GABP virus and cellular DNA decreases availability of GABP to cellular genes (see FIG. 114).

Whenever the concentration of viral N-boxes within a cell is greater than zero, microcompetition decreases the availability of GABP to cellular genes. ERK agents phosphorylate GABP and stimulate p300 binding. If the copy number of viral N-boxes is fixed, ERK agents stimulate transcription of GABP stimulated genes and suppress transcription of GABP suppressed genes.

(6) Note: ERK Agents and Latency

Assume a cell infected with the GABP virus “v.” Define latency (abortive replication, consistent infection) as existence of an upper limit the number of p300·GABP·N-boxᵥ complexes. Such limit prevents excessive viral replication, and sets a limit on the number of viral N-boxes in infected cells.

ERK agents stimulate formation of p300·GABP complexes on both viral and cellular N-boxes. However, under latency, the virus resists the stimulating effect of the ERK agent, and prevents the “excessive” formation of p300·GABP·N-boxᵥ complexes. Without latency, an ERK agent, with a larger stimulating effect on [p300·GABP·N-boxᵥ] compared to [p300·GABP·N-box_{G}] (G denotes a cellular gene), would, have accentuated microcompetition with the viral DNA instead of attenuating it.

To conclude: treatment with an ERK agent attenuates the effect of microcompetition with viral DNA as long as the virus continues to replicate under conditions of latency. See observations below relating to this note. See also examples in chapters on cancer, p 358, and treatment, p 444.

(7) JNK/SAPK Pathway

(a) Phosphorylation of GABP

JNK/SAPK is another signaling pathway that results in GABP phosphorylation (Hoffmeyer 1998, ibid) (see right side of FIG. 112).

Call each kinase that phosphorylates GABP, including ERK and JNK/SAPK, a GABP kinase.

b) Redox and N-box GABP

(1) Model: Redox Regulation of GABP N-box Binding

Let OS denote cellular oxidative stress and [GABP·N-box] the concentration of GABP bound to the N-box (or the probability that GABP is detected bound to the N-box). The following function presents the effect of oxidative stress on [GABP·N-box].

$$\begin{matrix}[ GABP \cdot N \text{-} box ] = & {f}_{OS} ( OS ) \\ & ( - )\end{matrix} \qquad Function 35$$

f_{OS} is called the “redox effect on GABP” model. Oxidative stress decreases binding of GABP to the N-box and consequently decreases transcription of GABP stimulated genes and increases transcription of GABP suppressed genes. Symbolically, for the GABP stimulated gene G,
↑[OS]→↓[GABP·N-box_{G}]→↓[mRNA_{G}]
Sequence of quantitative events 78: Predicted effect of oxidative stress on transcription of a GABP stimulated gene.

(2) Predictions and Observations

A study (Martin 1996⁹⁸¹) treated mouse 3T3 cells with diethyl maleate (DEM), a glutathione (GSH)-depleting agent, in the presence or absence of N-acetylcysteine (NAC), an antioxidant and a precursor of GSH synthesis, for 2 h. Following treatment, the cells were harvested and nuclear extracts were prepared in the absence of a reducing agent. GABP DNA binding activity was measured by electrophoretic mobility shift analyses (EMSA) using oligonucleotide probes containing a single N-box (AGGAAG) or two tandem N-boxes (AGGAAGAGGAAG). According to the redox effect on GABP model, [symbol]
[DEM]→↓[GSH]→↑[OS]→↓[GABP·N-box]
Sequence of quantitative events 79: Predicted effect of diethyl maleate (DEM) on the GABP·N-box complex. [symbol]
[DEM]+ [symbol] [NAC]→↑↓[GSH]→↑↓[OS]→↑↓[GABP·N-box]
Sequence of quantitative events 80: Predicted effect of diethyl maleate (DEM and N-acetylcysteine (NAC) on the GABP·N-box complex.

As expected, treatment of 3T3 cells with DEM decreased formation of the GABP heterodimers (GABPα·GABPβ) and the heterotetramer (GABPα₂·GABPβ₂) complexes on the single and double N-box probes (Martin 1996, ibid). Inhibition of GABP DNA binding activity by DEM treatment was prevented by the simultaneous addition of NAC. The decrease of GABP DNA binding activity was not due to loss of GABP protein since the amount of GABPα and GABPβ1 was unaffected by treatment with DEM or NAC. Treatment of nuclear extracts prepared from DEM-treated 3T3 cells with the antioxidant dithiothreitol (DTT) restored GABP binding activity, while treatment of 3T3 nuclear extracts with 5 mM oxidized glutathione (GSSG) nearly abolished GABP DNA binding. Based on these observations, Martin, et al., (1996, ibid) concluded that GABP DNA binding activity is inhibited by oxidative stress, i.e. GSH depletion.

The study also measured the effect of DEM treatment on the expression of transiently transfected luciferase (LUC) reporter constructs containing a TATA box with either an upstream double N-box or C/EBP binding site.

According to the “redox effect on GABP” model, [symbol]
[DEM]→↓[GSH]→↑[OS]→↓[GABP·N-box]→↓[mRNA_{LUC}]
Sequence of quantitative events 81: Predicted effect of diethyl maleate (DEM) on luciferase (LUC) repotrer constructs under control of a double N-box.

DEM treatment showed no effect on luciferase expression from C/EBP-TA-Luc after 6 or 8 h treatment. However, DEM treatment of cells transfected with double N-box-TATA-Luc decreased luciferase expression by 28% after 6 h, and 62% after 8 h. Based on these results, Martin, et al., (1996, ibid) further concluded that glutathione depletion inhibits GABP DNA binding activity, which decreases expression of GABP-regulated genes.

Taken together the results demonstrate that oxidative stress decreases GABP binding to the N-box, which, in turn, decreases transcription of GABP stimulated genes and increases transcription of GABP suppressed genes.

(3) Conclusions: “Excess Oxidative Stress”

Oxidative stress decreases binding of GABP to the N-box. Microcompetition with foreign DNA for GABP also decreases binding of GABP to the N-box. For GABP regulated genes sensitive to oxidative stress exclusively through GABP, the effect of microcompetition with foreign DNA on transcription is similar to the effect of oxidative stress. In other words, for this type of genes, microcompetition with foreign DNA can be viewed as “excess oxidative stress.”

c) Allocation Model of Transcription

(1) Model

Let G be a GABP regulated gene and “v” a GABP virus. The following function summarizes the effect of microcompetition with foreign DNA, GABP kinases, and redox on GABP.

Function 36

| [mRNA_{G}] = | f_{A-T}([DNA_{G-GABP}], | [DNA_{v-GABP}], | Affinity_{v/G}, | [GABPkinase^{phos}], | OS) |
|---|---|---|---|---|---|
| GABP stimulated gene | (+) | (−) | (−) | (+) | (−) |
| GABP suppressed gene | (−) | (+) | (+) | (−) | (+) |

For a GABP stimulated gene, microcompetition between the viral and cellular N-boxes for p300·GABP binding decreases transcription of the cellular gene (see (−) signs under [DNA_{v-GABP}] and Affinity_{v/G}). An increase in phosphorylation of a GABP kinase increases transcription (see the (+) sign under [GABPkinase^{phos}]), and an increase in oxidative stress decreases transcription (see the (−) sign under OS). For a GABP suppressed gene, the effects are reversed as indicated by the above function.

The independent variables in f_{A} function can be viewed as factors influencing allocation of a limited resource to the process of producing mRNA of a certain gene (hence the subscript A-T in f_{A-T} function, and the name “allocation model of transcription”). An increase in [DNA_{v-GABP}], or affinity of viral N-box to GABP, decreases allocation of p300 to G. An increase in GABP kinase phosphorylation increases affinity of the GABP complex to p300, and increases allocation of p300 to G. An increase in oxidative stress decreases binding of GABP to N-box_{G}, and decreases allocation of p300 to G.

(2) Predictions and Observations

(a) AChRδ and ε

(i) GABP Stimulated Gene

Binding of GABP to the N-box in the nicotinic acetylcholine receptor δ and ε (AChRδ and ε) genes stimulates transcription (Schaeffer 1998⁹⁸², Duclert 1996⁹⁸³, Koike 1995⁹⁸⁴j.

(ii) GABP Kinase as Stimulator

Heregulin is an ERK agent. According to the “allocation model of transcription,” treatment with heregulin should stimulate AChRδ and ε transcription (see (+) sign under [GABPkinase^{phos}] in f_{A-T} above). As expected, Fromm 1998⁹⁸⁵, and Tansey 1996⁹⁸⁶ reported observations showing increased transcription of both the AChRδ and AChRε genes following treatment with heregulin.

Moreover, Schaeffer 1998 (ibid) reported that heregulin treatment of chick primary myotubes increased phosphorylation of GABPα and GABPβ, and dominant-negative mutants of GABPα and GABPβ blocked the heregulin-elicited activation of AChRδ and ε transcription. Fromm 1998 (ibid) produced transgenic mice carrying a fusion between the mouse AChRδ gene and the hGH gene. The study showed that 181 bp of the 5′ flanking DNA from AChRδ is sufficient to confer synapse-specific expression. However, transgenic mice carrying a transgene with a mutation in the N-box showed no synaptic expression. The results in these two studies are consistent with the proposed “allocation model of transcription.”

Gramolini 1999⁹⁸⁷ reported similar results with utrophin, another GABP regulated gene. Heregulin also stimulated transcription of utrophin, while site-directed mutagenesis of a single N-box in the utrophin promoter inhibited the effect. Moreover, over expression of heregulin, or GABPα and β, in cultured myotubes resulted in an N-box-dependent increase in utrophin promoter activity.

10. Signal Resistance

a) Model

(1) Resistance and Hyper-Emia

Definition: Resistance

Assume an agent L that produces the effect Y in O. O will be called “L resistant” if a given concentration of L produces a smaller Y effect in O relative to control. Examples of L resistance include insulin resistance and leptin resistance.

Notes:

1. O can be a cell or a patient.

2. From the definition, it follows that an increase in blood glucose with similar or elevated levels of insulin is considered insulin resistance.

Definition: Hyper-Emia

Assume an agent L that produces the effect Y in O. A sustained increase of L in O will be called hyper-L-emia. Examples of hyper-L-emia include hyperinsulinemia and hyperleptinemia.

Note: Hyper-emia is usually reserved for patients.

(2) Microcompetition with Foreign DNA and Resistance

Let AGENT be a GABP kinase agent that produces the effect Y in O. If the Y effect is dependent on transcription of a GABP regulated gene X in O, denoted (AGENT, GABP, X, Y), then microcompetition with foreign DNA for GABP in O results in AGENT resistance in O.

Under conditions of microcompetition for foreign DNA in O, a given concentration of AGENT produces a smaller concentration of X, and, therefore, smaller Y effect. The conclusion will be called the “microcompetition model of resistance.”

(3) Microcompetition and Hyper-Emia

(a) Control

Let AGENT be a GABP kinase agent and let C be a protein. If expression of AGENT depends on expression of C, C will be called “control” for AGENT. If an increase in C decreases expression of AGENT, or increases its degradation, C will be called a “negative control” and the effect on AGENT will be called “feedback inhibition.”

Let AGENT be a GABP kinase agent. If GABP stimulates C, C will be called a “GABP stimulated” control. Consider FIG. 115.

AGENT phosphorylates GABP (step 1 and 2), which increases transcription of C (step 3), which, in turn, decreases expression of the GABP kinase agent (step 4).

(b) Effect of Microcompetition with Foreign DNA

If AGENT is a GABP kinase agent, and C is a GABP stimulated gene and a negative control for AGENT in O, then microcompetition with foreign DNA for GABP in O results in hyper-AGENT-emia.

As a GABP kinase agent, AGENT phosphorylates the pool of GABP molecules, which increases expression of C, which, in turn, suppresses AGENT expression. Microcompetition with foreign DNA decreases the size of the GABP pool in O, or the amount of GABP available to stimulate C. Therefore, microcompetition with foreign DNA diminishes the increase in control C, which decreases the suppression effect on AGENT resulting in elevated concentrations of AGENT. In FIG. 115, microcompetition with foreign DNA decreases the size of the arrows in step 2, 3, and 4 (i.e. the magnitude of the indicated increases). The conclusion will be called the “microcompetition model of hyper-emia.”

(c) Special Case

What if the identity of the negative control is unknown, can hyper-emia be deduced? Consider the following special case.

Let AGENT be a GABP kinase agent. Every protein R, such that R is an element of the signaling cascade between AGENT and GABP, will be called a “GABP kinase receptor” for AGENT. In other words, AGENT activates R, which, in turn, activates GABP. For example, the leptin long receptor is a GABP kinase receptor for leptin, and metallothionein is a GABP kinase receptor for zinc.

Let R be a receptor in the AGENT to GABP pathway, denoted (AGENT, GABP). If expression of R is stimulated by GABP, R will be called a “sensitized receptor.” In the (OT, OTR, GABP) pathway, the receptor OTR is stimulated by GABP (Hoare 1999, ibid). In (zinc or copper, hMT-II_{A}, GABP), hMT-II_{A} is a receptor stimulated by GABP (see discussion above). In (IL-2, IL-2Rβ, γc, GABP), IL-2Rβ and γc are two receptors stimulated by GABP (Lin 1993, ibid, Markiewicz 1996, ibid).

GABP stimulation of a receptor amplifies the intensity of the signal produced by AGENT by increasing the sensitivity of the pathway to a given concentration of AGENT or the probability that a low concentration of AGENT produces a desired metabolic effect. Consider FIG. 116.

An increase in AGENT stimulates the pathway that increases phosphorylation of GABP (step 1 and 2 in the figure). Phosphorylation of GABP stimulates transcription of R₁, the sensitized receptor (step 3). The new R₁ receptors increase the pathway sensitivity to the AGENT, that is, the new R₁ receptors increase the probability of AGENT binding to R₁. The resulting increase in binding of AGENT to R₁ further increases the number of phosphorylated GABP molecules (step 4) in a positive feedback loop.

Note that some AGENTS stimulate transcription of GABP directly, turning GABP itself into a sensitized receptor. Consider the following examples. Western blot analysis of heregulin treated and untreated cells showed a 2-fold increase in GABPα protein level in treated cells compared to controls, while GABPβ was unaffected (Schaeffer 1998, ibid). Treatment with interferon-γ (IFNγ) (Tomaras 1999, ibid), and PMA (Bottinger 1994, ibid), produced similar effects on GABP transcription.

If R is a sensitized receptor in (AGENT, GABP), and R directly binds AGENT, then microcompetition with foreign DNA for GABP in O results in hyper-AGENT-emia regardless of the control position in the pathway. FIG. 117 illustrates this special case.

Since the sensitized receptor R binds AGENT, any control must be downstream from GABP. In such pathway, microcompetition with foreign DNA results in hyper-AGENT-emia regardless of the control position in the pathway.

Consider the following two pathways (OT, OTR, GABP), and (zinc or copper, hMT-II_{A}, GABP). In these pathways, the sensitized receptor directly binds the GABP kinase, agent. Therefore, the control must be down stream from the sensitized receptor, and the pathways must show hyper-emia under conditions of microcompetition with foreign DNA. In contrast, the pathway (IL-2, IL-2Rβ, γc, GABP) is different (see below).

Note: The pathway (LPS, CD18, GABP) operates in the opposite direction. CD18 is a GABP suppressed gene (see chapter on transefficiency, p 140). Therefore, elicitation of a bio-equivalent reaction requires a lower concentration of LPS in a cell infected by a GABP virus compared to non-infected cells.

b) Resistance in Obesity

(1) Catecholamine

(a) HSL Regulation

Catecholamines bind β₁-, β₂-, and β₃-adrenergic receptors (β₁AR, β₂AR, and β₃AR, respectively), and α₂ adrenergic receptors (α₂AR).

(i) Transcription

Activation of β₂AR (Maudsley 2000⁹⁸⁸, Pierce 2000⁹⁸⁹, Elorza 2000⁹⁹⁰, Luttrell 1999⁹⁹¹, Daaka 1998⁹⁹²), or β₃AR (Cao W 2000⁹⁹³, Gerhardt 1999⁹⁹⁴, Soeder 1999⁹⁹⁵), activates ERK, which, in turn, phosphorylates GABP, which binds p300, resulting in increased HSL transcription.

Symbolically,
↑[Catecholamine]→↑[p300·GABP·N-box_{HSL}]→↑[mRNA_{HSL}]→↑[Lipolysis]
Sequence of quantitative events 82: Predicted effect of catecholamine on rate of liposys.

The same sequence of quantitative events can be presented symbolically using parenthesis: (catecholamine, GABP, HSL, lipolysis).

(ii) Post-Translation

Activation of β₁AR, β₂AR, β₃AR activates the cAMP dependent protein kinase A (PKA). PKA phosphorylates HSL, resulting in increased hydrolytic activity against triacylglycerol and cholesteryl ester substrates. Insulin deactivates HSL via protein phosphatases or by inhibition of protein kinase A.

(b) Resistance

(i) Prediction

Assume obesity results from an infection with a GABP virus. Specifically, assume the virus infects adipocytes. Consider the (catecholamine, GABP, HSL, lipolysis) pathway. According to the microcompetition model of resistance, the infected adipocytes should show catecholamine resistance.

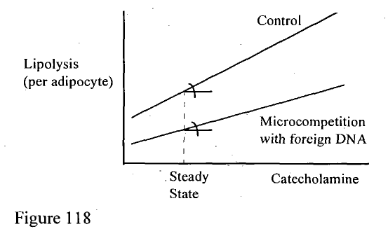

Since microcompetition with foreign DNA decreases HSL expression, and HSL is rate limiting in triacylglycerol and diacylglycerol hydrolysis, microcompetition with foreign DNA should decrease steady state lipolysis. As ERK agents, agonists of β₂AR and β₃AR, specifically catecholamines, stimulate HSL transcription. Microcompetition with foreign DNA should also attenuate the stimulated increase in HSL transcription, and, therefore, impair stimulated lipolysis. These predictions are summarized in FIG. 118.

At steady state, microcompetition with foreign DNA should decrease lipolysis. Moreover, microcompetition with foreign DNA should also decrease the slope of the lipolysis line, that is, under conditions of microcompetition with foreign DNA, higher catecholamine stimulation should show a greater lipolysis deficiency (a larger vertical difference between the two lines). Consider the following observations.

(ii) In Vitro Observations

A study (Hellstrom 1996⁹⁹⁶) treated abdominal subcutaneous adipocytes from 13 non-obese subjects with at least one first-degree relative with a body mass index of 27 kg/m² or more (Hob) and 14 controls (Hnorm) with several agents. Specifically, the study used norepinephrine, a major endogenous lipolytic agent, isoprenaline, a non-selective beta-adrenoceptor agonist, forskolin, a direct activator of adenylyl cyclase, and dibutyryl cyclic AMP, an activator of protein kinase, and thereby HSL.

Isoprenaline (Shimizu 1997⁹⁹⁷), dibutyryl cAMP (Shimizu 1997, ibid), and forskolin (Yarwood 1996⁹⁹⁸) activated ERK in adipocytes, and isoprenaline activated ERK in CHO/K1 cells expressing the human β₃AR (Gerhardt 1999, ibid). The observations indicate that norepinephrine, isoprenaline, forskolin, and dibutyryl cAMP are ERK agents in adipocytes. According to the microcompetition model of resistance, if obese adipocytes harbor a GABP virus, the cells should show norepinephrine, isoprenaline, forskolin, and dibutyryl cAMP resistance.

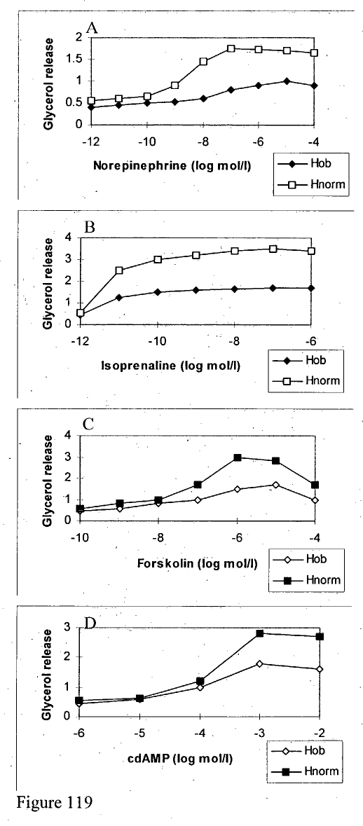

FIG. 119 presents the effect of the treatments on glycerol release (pmol·cell·2h⁻¹). As predicted, the average rate of lipolysis induced by all four treatments was decreased by about 50% (p from 0.001 to 0.01) in adipocytes from subjects with a family trait of obesity compared to controls. Moreover, as predicted, an increase in agonist concentration increased the lipolysis deficiency (compare the figure presenting the predicted resistance and the figures presenting the observed resistance).

Hellstrom 1996 (ibid) also measured maximum HSL activity and mRNA at steady state. As predicted, Hob showed 50% decrease in maximum activity (p<0.05), and 20% decrease in mRNA levels (p>0.05, not significant) in Hob. The study did not measure HSL mRNA following stimulation. Since the lipolysis deficiency increases with stimulation, it is reasonably to predict that following stimulation, the difference in mRNA between obese and control will turn statistically significant, however such prediction was not tested.

The following two studies, instead of adipocytes lipolysis at steady state, measured maximum adipocyte lipolysis following 2 h incubation with various agonists. Large 1999 (ibid) treated abdominal subcutaneous adipocytes from 34 obese drug-free and otherwise healthy males or females, and 14 non-obese controls, with the ERK agent isoprenaline, or dibutyryl cAMP. Hellstrom 2000⁹⁹⁹ treated abdominal subcutaneous adipocytes from 60 obese and 67 non-obese subjects, age 19-60 y, with the ERK agent isoprenaline, dibutyryl cAMP, or forskolin. According to the microcompetition model of resistance, if obese adipocytes harbor a GABP virus, the cells, in both studies, should show isoprenaline, dibutyryl cAMP, and forskolin resistance, that is, a decrease in maximum adipocyte lipolysis.

As predicted, Large 1999 (ibid) observed a 40-50% decrease in maximum isoprenaline-, and dibutyryl cAMP-induced glycerol release in the obese group, when expressed per g lipid. Hellstrom 2000 (ibid) observed a 50% decrease in maximum isoprenaline-, dibutyryl cAMP-, and forskolin-induced glycerol release in the obese group.

The in vitro observations in Hellstrom 1996 (ibid), Large 1999 (ibid), and Hellstrom 2000 (ibid) are consistent with a GABP viral infection in obesity resulting in microcompetition-induced resistance.

(iii) In Vivo Observations

To examine the effect of epinephrine on lipolysis in obesity, a study (Bougneres 1997¹⁰⁰⁰) infused epinephrine in a stepwise manner at fixed doses of 0.75 and then 1.50 μg/min to 9 obese children (160±5% ideal body weight) aged 12.1±0.1 yr during the dynamic phase of fat deposition, and in 6 age-matched non-obese children. As an in vivo lipolysis index, the study used glycerol flux. Epinephrine is an ERK agent. According to the microcompetition model of resistance, if obese adipocytes harbor a GABP virus, the obese subjects should show epinephrine resistance, that is, decreased glycerol flux.

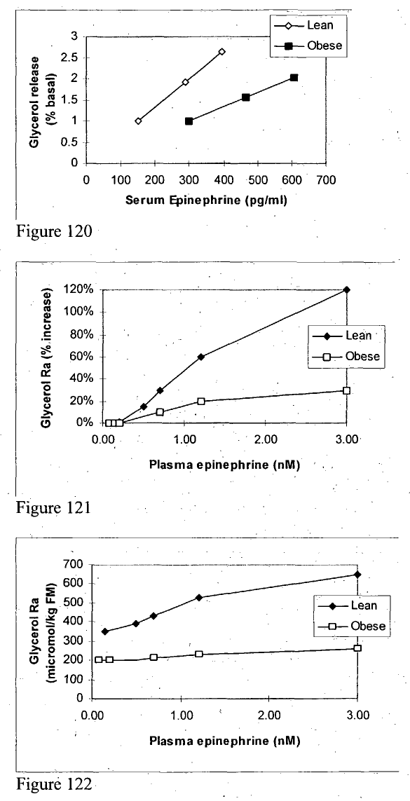

FIG. 120 presents the observed relation between epinephrine infusion and glycerol release.

As predicted, the results showed a 30% decrease in the rate of glycerol release per unit fat mass in obese children.

Another study (Horowitz 2000¹⁰⁰¹) measured lipolytic sensitivity to epinephrine in 8 lean [body mass index (BMI):21±1 kg/m²] and 10 upper body obese (UBO) women (BMI: 38±1 kg/m²; waist circumference>100 cm). All subjects underwent a four-stage epinephrine infusion (0.00125, 0.005, 0.0125, and 0.025 microgram·kg fat-free mass⁻¹·min⁻¹) plus pancreatic hormonal clamp. Glycerol rates of appearance (Rₐ) in plasma were determined by stable isotope tracer methodology. According to the microcompetition model of resistance, if obese adipocytes harbor a GABP virus, the obese subjects should show epinephrine resistance, that is, decreased glycerol release.

FIG. 121 presents the observed percent change in glycerol release as a function of plasma epinephrine concentration. FIG. 122 represents the same results in terms of total glycerol release per fat mass (FM).

As predicted, the results showed decreased glycerol rates of appearance (Rₐ) in plasma of obese women. The in vivo observations in Bougneres 1997 (ibid) and Horowitz 2000 (ibid) are consistent with a GABP viral infection in obesity resulting in microcompetition-induced resistance.

(2) Oxytocin (OT)

OT is a nine amino acid peptide synthesized in hypothalamic neurons and transported down axons of the posterior pituitary for secretion into blood. Oxytocin is also secreted within the brain and from a few other tissues, including ovaries and testes. The oxytocin receptor (OTR) is expressed on the surface of breast and uterine smooth muscle cells. OTR is a GABP stimulated gene (Hoare 1999, ibid).

OT stimulates the contraction of uterine smooth muscle at birth. During later stages of gestation, uterine smooth muscle cells, especially in myometrium, increase OTR transcription (Kimura 1996¹⁰⁰²); During labor, oxytocin facilitates parturition by stimulating contraction of uterine smooth muscle. In cases where uterine contractions are insufficient to complete delivery, physicians and veterinarians sometimes administer oxytocin (“pitocin”) to further stimulate uterine contractions.

GABP viruses infect uterine smooth muscle cells. For instance, Myerson 1984¹⁰⁰³ reported detection of cytomegalovirus (CMV) DNA in myometrium. Other GABP viruses, such as Epstein-Barr virus (EBV), were also detected in the cervix (Voog 1997¹⁰⁰⁴, Taylor 1994¹⁰⁰⁵).

Assume obesity results from an infection with a GABP virus. Specifically, assume the virus infects uterine smooth muscle cells. Consider the (oxytocin, OTR, GABP, uterine contractions) pathway. According to the microcompetition model of resistance, the infected cells should show oxytocin resistance. Consider the following observations.

As predicted, Johnson 1987¹⁰⁰⁶ reported that obese patients weighing at least 113.6 kg (250 pounds) during pregnancy showed a significantly increased need for oxytocin stimulation of labor compared to age and parity matched controls.

OT also stimulates milk ejection (milk letdown). Initially, milk is secreted into small sacs within the mammary gland called alveoli that are surrounded by smooth muscle (myoepithelial) cells. Oxytocin stimulates the contraction of myoepithelial cells causing ejection of milk into ducts and cisterns.

Assume obesity results from an infection with a GABP virus. Specifically, assume the virus infects breast smooth muscle cells. Consider the (oxytocin, OTR, GABP, milk ejection) pathway. According to the microcompetition model of resistance, the infected cells should show oxytocin resistance. Consider the following observations.

Chapman 1999¹⁰⁰⁷ identified obesity as a risk factor for the delayed onset of lactation. Donath 2000¹⁰⁰⁸ observed that 82.3% of obese mothers (BMI≧30) initiated breastfeeding compared to 89.2% of controls (BMI of 20-25). There was also a significant difference between the mean and median duration of breastfeeding of obese and non-obese mothers. Controlling for maternal smoking, age, and other socio-demographic factors, which often co-vary with maternal obesity and breast-feeding, did not change the results. Hilson 1997¹⁰⁰⁹ performed a logistic regression analysis of mothers who ever put their infants to the breast (n=810). The results showed that overweight (BMI 26.1-29.0) or obese women (BMI>29.0) had less success initiating breast-feeding than did normal-weight counterparts (BMI<26.1). Moreover, a proportional-hazards regression revealed higher rates of discontinuation of exclusive breast-feeding in overweight and obese women, and higher discontinuation of breast-feeding to any extent in overweight and obese women. Controlling for parity, socioeconomic status, maternal education, and other factors, which often co-vary with maternal obesity and breast-feeding, did not change these results. According to Hilson, et al., (1997, ibid): “these results suggest that excessive fatness in the reproductive period may inhibit lactational performance in women.”

The increased need for oxytocin stimulation of labor and the decreased lactational performance in obesity are consistent with the microcompetition model of oxytocin resistance in obesity.

(3) Insulin

The association of obesity with type II diabetes is well established. Specifically, the major basis for the link is the ability of obesity to cause insulin resistance (see recent review on obesity and insulin resistance in Kahn 2000¹⁰¹⁰). The term insulin resistance usually means decreased insulin-stimulated glucose transport and metabolism in adipocytes and skeletal muscle and impaired suppression of hepatic glucose output.

Consider glucose transport in adipocytes as example. Insulin is an ERK agent. Let X denote a GABP stimulated gene that increases glucose transport in adipocytes. Assume obesity results from an infection with a GABP virus. Specifically, assume the virus infects adipocytes. Consider the (insulin, GABP, X, glucose transport) pathway. According to the microcompetition model of resistance, the infected cells should show insulin resistance. Note that the model derives the same conclusion for GABP stimulated insulin receptors (a case similar to oxytocin, see above).

As expected, recent studies reported that an infection with human immunodeficiency virus (HIV), a GABP virus, results in insulin resistance and weight gain (Dube 2000¹⁰¹¹). The observation is consistent with the microcompetition model of insulin resistance in obesity.

c) Hyper-Emia in Obesity

(1) Oxytocin (OT)

The oxytocin receptor (OTR) is a GABP stimulated gene (for references see chapter on microcompetition, p 128). Consider the (OT, OTR, GABP) pathway. As a GABP stimulated gene, OTR is a sensitized receptor, which binds directly to the GABP kinase agent. In such pathway, microcompetition with foreign DNA results in hyper-emia regardless of the negative control position in the pathway (see discussion above).

Let Q be a cell that expresses OTR. Assume obesity is associated with a GABP viral infection in O cells. According to the microcompetition model of hyper-emia, obese patients should show hyper-oxytocin-emia, specifically, elevated levels of plasma oxytocin. Consider the following observation.

A study (Stock 1989¹⁰¹²) compared plasma oxytocin levels in obese and control subjects. As expected, the results showed 4-fold higher plasma oxytocin levels in obese individuals. Following weight loss induced by gastric banding, the obese subjects showed decreased plasma oxytocin levels. However, even after weight loss, oxytocin levels were still markedly higher than in controls.

(2) Zinc and Copper

The (zinc or copper, HMT-II_{A}, GABP) pathway is similar to (OT, OTR, GABP), and, therefore, should show hyper-emia under conditions of microcompetition with foreign DNA regardless of the negative control position in the pathway. Consider the following observations.

A study (Yakinci 1997¹⁰¹³) measured serum zinc, copper, and magnesium levels in healthy and obese children using atomic absorption spectrophotometry. Serum zinc and copper levels of obese children (mean values 102.40±2.78 micrograms/dL and 132.34±1.79 micrograms/dL, respectively) were markedly higher than controls (mean values 80.49±2.98 micrograms/dL, and 107.58±1.62 micrograms/dL, respectively). Serum copper concentrations were also significantly higher in obese children compared to controls. Another study (D'Ocon 1987¹⁰¹⁴) determined serum zinc and copper levels in 140 diabetic patients and 162 healthy controls. A subgroup of those patients, classified as overweight (greater than 15% increase in body weight), showed a statistically significant increase in zinc levels. Taneja 1996¹⁰¹⁵ measured the concentration of zinc in hair of obese men and women. The results showed a positive linear correlation between body weight, or body weight/height ratio, and hair zinc concentration. The correlation was stronger in men.

The observations in Yakinci 1997 (ibid), D'Ocon 1987 (ibid), and Taneja 1996 (ibid) are consistent with the microcompetition model of hyper-emia.

(3) Insulin and Leptin

The association of hyperinsulinemia and hyperleptinemia with obesity is well established.

Consider the ERK agent insulin. Let Cᵢₙₛᵤₗᵢₙ denote a GABP stimulated negative control, and O_{insulin-c} a cell that expresses the control. Assume obesity results from an infection with a GABP virus. Specifically, assume the virus infects O_{insulin-c} cells. According to the microcompetition model of hyper-emia, obese patients should show hyperinsulinemia.

A similar argument can be made with regard to leptin. The observed hyperleptinemia in MT-I and MT-II null mice (see above) is consistent with the microcompetition model of hyperleptinemia in obesity.

Note that other GABP kinase agents also show hyper-emia in obesity, for instance, estron (E1), estradiol (E2), estriol (E3) (Cauley 1994¹⁰¹⁶, Cauley 1989¹⁰¹⁷, de Waard 1982¹⁰¹⁸), and interleukin 6 (IL-6) (Pickup 1998¹⁰¹⁹, Pickup 1997¹⁰²⁰).

Notes about non hyper-emic GABP kinase agents.

1. IL-2β is an ERK agent with receptors, interleukin 2 receptor β chain (IL-2Rβ) and IL-2 receptor γ-chain (γc), which are GABP stimulated genes (Markiewicz 1996, ibid, Lin 1993, ibid). Microcompetition with foreign DNA for GABP decreases transcription of these receptors. Since any control in the pathway has to be downstream from the receptors, microcompetition with foreign DNA for GABP diminishes expression of the control. The decreased expression of the control decreases its suppressive effect on IL-2p, which, in turn, elevates concentration of IL-2β. However, IL-2β itself is a GABP stimulated gene (Avots 1997, ibid). Therefore, microcompetition with foreign DNA also decreases transcription of IL-2,1. The combined effect of diminished suppression of transcription and diminished transactivation of transcription can result in a decline, increase, or no change in concentration of IL-2β.

The implicit assumption regarding the GABP kinase agents discussed above is independence between GABP transcription and agent transcription. IL-2β violates the condition, resulting in an inconclusive prediction.

11. Osteoarthritis

a) Introduction

Osteoarthritis is the most common form of chronic joint disease affecting millions of people in the United States. The prevalence of the disease after the age of 65 years is about 60% in men and 70% in women. Considering the cost of diagnosis, therapy, side effects, and lost productivity, osteoarthritis is one of the more expensive and debilitating diseases in the United States. Osteoarthritis is characterized by progressive loss of articular cartilage and bony overgrowth at the joint margins.

This chapter presents a model that relates microcompetition with foreign DNA and osteoarthritis.

b). Collagen Type I α2 Chain Gene (COL1A2)

(1) COL1A2 is a Microcompetition-Suppressed Gene

A study (Allebach 1985¹⁰²¹) infected skin fibroblasts with a temperature sensitive Rous Sarcoma Virus (ts-RSV), and measured the amount of endogenous COL1A2 RNA in cells grown at either permissive (T) or nonpermissive (N) temperatures for viral replication. Assume that the Rous Sarcoma Virus and COL1A2 bind the same limiting complex, and that the complex stimulates COL1A2 transcription. Then, a shift from nonpermissive to permissive temperature, increases RSV replication and RSV DNA copy number, increases microcompetition between the foreign DNA and COL1A2 promoter/enhancer, which should decrease COL1A2 RNA levels.

As expected, the observations showed a 5-fold decrease in COL1A2 RNA at the permissive relative to nonpermissive temperature. Earlier experiments reported by the same laboratory demonstrated a 3.3-fold decrease of endogenous gene expression.

Related experiments were carried out with WI-38 human lung fibroblasts transformed by a clone of SV40. Endogenous COL1A2 mRNA was absent in SV40 transformed cells, whereas mRNA for the α1(I) chain (COL1A1) was detected on the same Northern blot (Parker 1989¹⁰²²), demonstrating the specificity of the microcompetition effect. That study eliminated several potential reasons for the observed decrease in COL1A2 mRNA in the transformed cells. The chromosomes, which normally carry the COL1A2 and COL1A1 genes, appeared to be normal and restriction enzyme mapping of the COL1A2 gene in the transformed cells did not show any gross insertion of the viral genome within the gene or its promoter. Finally, methylation analyses of the promoter and 3′ regions of the gene showed no detectable hypermethylation. Taken together the observations are consistent with the contention that microcompetition with the SV40 promoter/enhancer is responsible for the decreased expression of the COL1A2 gene.

Moreover, a study (Czuwara-Ladykowska 2001¹⁰²³) identified 5 ETS binding sites (EBSs) in the (−353, −180) region of the COL1A2 promoter. The study showed that ets-1 and ets-2 transactivate the COL1A2 promoter in dermal fibroblasts (Czuwara-Ladykowska 2001, ibid, FIG. 7), and that ets-1 weakly binds to a promoter fragment, which contains the EBS at (−290, −279). Although the study never tested GABP, the observations of active EBSs support possible transactivation by GABP.

The observations in these studies suggest that microcompetition between the DNA of a GABP virus and COL1A2 promoter decreases COL1A2 transcription. Symbolically,
↑[N-boxᵥ]→↓[mRNA_{COL1A2}]
Sequence of quantitative events 83: Predicted effect of foreign N-boxes on mRNA levels of collagen type I α2 chain gene (COL1A2).

(2) COL1A2 Deficiency and Osteoarthritis

The following sections use studies on Ehlers-Danlos syndrome type-VII (EDS type-VII) to deduce the effect of a GABP viral infection on the probability of developing osteoarthritis.

(a) COL1A2 and Hypermobility of Joints

A hypermobile joint has a range of motion that exceeds the standard for a normal joint. A primary cause of hypermobility is ligamentous laxity (Grahame 1999¹⁰²⁴). A heterozygous mutation in the COL1A2 gene, which decreases COL1A2 expression, increases hypermobility of joints in EDS type-VII patients (Byers 1997¹⁰²⁵, Giunta 1999¹⁰²⁶) Symbolically,
↓[mRNA_{COL1A2}]→↑Hypermobility
Sequence of quantitative events 84: Predicted effect of COL1A2 mRNA on hypermobility.

(b) Hypermobility and Osteoarthritis

A study of EDS type-VII patients found that 16 out of 22 over the age of 40 show osteoarthritis (OA) in one or more joints (referenced in Grahame 1989¹⁰²⁷). In the general population, evidence is more circumstantial. Some of the evidence was produced by the Leeds groups, which identified a likely association between joint laxity and osteoarthritis. The study compared 50 women with symptomatic OA to age matched controls and found a direct correlation between the degree of hypermobility and OA (Scott 1979¹⁰²⁸)

The association between hypermobility and osteoarthritis was studied in specific joints. Sharma 1999¹⁰²⁹ reported greater laxity in uninvolved knees of OA patients compared to older controls. Based on this observation, Sharma, et al., (1999, ibid) concluded that at least some of the increased laxity of OA may predate the disease. Jonsson 1996¹⁰³⁰ compared 50 female patients with clinical thumb base (first carpometacarpal joint) OA to age matched controls. The results showed increased prevalence of hypermobility in patients compared to controls. The authors also mention another study with 100 patients (including both males and females) that found a direct correlation between hypermobility and clinical severity of thumb base OA. Based on these observations, Jonsson, et al., (1996, ibid) concluded that a causal relation might exist between articular hypermobility and thumb base OA.

To summarize, observations show a direct relation between hypermobility and osteoarthritis. Symbolically,
↑Hypermobility→↑[Osteoarthritis]
Sequence of quantitative events 85: Predicted effect of hypermobility on susceptibility to osteoarthritis.

(3) Logical Summary

According to the principle of transitive deduction:
If (↑[N-boxᵥ]→↓[mRNA_{COL1A2}]) AND (↓[mRNA_{COL1A2}]→↑Hypermobility) AND (↑Hypermobility→↑[Osteoarthritis]) Then (↑[N-boxᵥ]→↑[Osteoarthritis])

A three step transitive deduction suggests that an infection with a GABP virus should lead to osteoarthritis.

c) Osteoarthritis and Obesity

(1) Vulnerable Joints

Matrix components of interarticular fibrocartilage tissues (menisci) show a relatively high concentration of collagen type I, about 55-65% of dry weight. Meniscus tissues are found in various joints, such as, temporomandibular, sternoclavicular, acromioclavicular, wrist, and knee. Connecting fibrocartilages, such as intervertebral discs, also show high concentration of collagen type I.

Joints with relatively high concentrations of collagen type I will be called “vulnerable” to OA, indicating increased susceptibility to hypermobility. Specifically, the temporomandibular, sternoclavicular, acromiocalvicular, wrist, knee, and lumber joints are vulnerable joints.

(2) Hypermobility and Obesity

A latent infection with a GABP virus decreases COL1A2 expression, which, in turn, increases hypermobility of joints, especially in vulnerable joints. Since an infection with a GABP virus results in obesity (see chapter on obesity, p 310), obese people should show hypermobility in vulnerable joints. Consider the following observations.

Lumbar joints are vulnerable. A study (Batti'e 1987¹⁰³¹) used a modified Schober test to examine lumbar mobility. To perform the test, a subject was first asked to stand erect. While erect, three marks were placed on the subject's skin overlaying the lumbosacral spine. The first mark was placed at the lumbosacral junction, the second was placed 5 cm below the first, and the third was placed 10 cm above the first. The subject was then asked to bend forward as far as possible, as though to touch his or her toes. The new distance between the second and third mark was measured. Lumbar mobility was defined as the difference between the measurement at bend position and the initial 15 cm distance marked on the subject back. The study group included 2,350 men and 670 women between the ages of 21 and 67 years.

The results showed that obesity (defined as weight/height) markedly affected flexibility. An increase of one standard deviation in obesity resulted in a 0.4 cm increase in lumbar mobility. Lumbar mobility declined with age, specifically females in their 60's showed a 0.42 cm decrease in the modified Schober measurement compared to females in their 20's. Men showed a 1.04 cm decrease over the same age interval. The increased flexibility demonstrated by most obese subjects (top 16%, or 1 SD of weight/height subjects) was equal to the difference in flexibility associated with a 40 year age difference in female (0.4 cm compared to 0.42 cm), and almost half the difference associated with that age difference in men (0.4 cm compared to 1.04 cm).

These observations in Batti'e 1987 (ibid) are consistent with the predicted association between hypermobility in vulnerable joints and obesity.

(3) Osteoarthritis and Obesity

Hypermobility increases susceptibility to osteoarthritis. Therefore, obese people should show increased susceptibility to osteoarthritis, especially in vulnerable joints.

A study (Cicuttini 1996¹⁰³²) compared OA disease traits in different joints of female twins aged 48-70. The results showed that, in twins, an increase in body weight increases the likelihood of developing osteoarthritis in the knee, in both the tibiofemoral joint (TFJ) and patellofemoral joint (PFJ), and in the hand in the first carpometacarpal joint (CMC I). Specifically, after adjustment for other potential risk factors, every 1 kg increase in body weight resulted in a 14% increased risk of developing TFJ osteophytes, 32% increased risk of developing PFJ osteophytes, and a 10% increased risk of developing CMC osteophytes, in the heavier twin. The study notes that since weight differences were also observed in asymptomatic twins, weight gain predates OA, and therefore does not result from OA. Specifically, the weight gain does not result from the decreased mobility associated with osteoarthritic pain and discomfort.

Note: Since the study compared twins, the observed association between obesity and OA is independent of genetic factors, and specifically, is inconsistent with a mutation as an underlying cause.

Another study (Carman 1994¹⁰³³) started in 1962 with baseline examinations of various clinical, biochemical, and radiologic characteristics, including obesity, measured as an index or relative weight. In 1985, the study examined 1,276 participants, 588 males and 688 females, ages 50-74, for symptoms of osteoarthritis. The results showed an increase in the likelihood of developing osteoarthritis of the hand, over the 23-year study period, with an increase in the index measuring baseline relative weight. Higher baseline relative weight was also associated with greater subsequent severity of the disease. The study also found that during the 23-year period, most subjects gained weight. However, the increase in body weight was not associated with either the likelihood of developing osteoarthritis of the hand, or the severity of the disease. Based on these observations, Carman, et al., (1994, ibid) concluded that although there is an association between obesity and OA, OA is not a result of weight gain.

In obesity, some joints seem to be susceptible to osteoarthritis while others are protected. Knees and the thumb base, for instance, are often damaged, while hips are disease free. Since both the knee and hip are weight-bearing joints, the difference in susceptibility to osteoarthritis indicates a cause other than simple mechanical wear-and-tear. The pattern of OA in obesity is also inconsistent with a general metabolic cause for the disease. A metabolically induced deterioration of cartilage should result in small differences in the severity of OA between joints, unlike the differences observed in joints of obese people. van Saase 1998¹⁰³⁴ called the pattern of OA in obesity “strange,” and claimed that “whatever the final explanation for the etiology of OA, we believe that it will have to take into account the strange pattern of the association between OA and obesity.”

(4) Summary

The observations in the above studies suggest three conclusions. First, obesity is associated with osteoarthritis, but only in specific joints—van Saase's “strange” list of susceptible joints. Second, obesity and osteoarthritis do not a result from of each other. Third, the association between obesity and osteoarthritis is independent of genetic factors. Microcompetition with foreign DNA as the origin of obesity (see chapter on obesity, p 310) and OA (this chapter) is consistent with all three conclusions. First, van Saase's list of “strange” joints coincides with the list of vulnerable joints. Second, both obesity and OA result from a viral infection and not from each other. Last, the association between obesity and OA results from a viral infection and not from a genetic mutation.

d) Obstructive Sleep Apnea (OSA) and Obesity

Obesity is associated with hypermobility of vulnerable joints including the temporomandibular joint. Therefore, in obesity the temporomandibular joint should show hypermobility. Consider the following observations.

A study (Ferguson 1997¹⁰³⁵) compared the mandible and tongue protrusion of obese patients to controls. A subject was asked to protrude the mandible or tongue as far forward as possible (MAX). The midpoint between maximum protrusion and the position were the tongue tip is resting between the incisors was denoted “50%.” Then, the study measured the cross-sectional area of the oropharynx during MAX and 50% tongue protrusion. The results showed a greater relative increase in oropharyngeal cross-sectional area in obese subjects compared to controls. Since increased oropharyngeal cross-sectional area indicates an increased capacity for mandibular protrusion, the observations are consistent with hypermobility of the temporomandibular joint in obesity.

During sleep, the tonic activity of the masseter decreases. In a supine position, the mandible drops, and mouth opens. A hypermobile temporomandibular joint lets the mandibular drop further and the mouth open wider than a normal joint.

A study (Miyamoto 1999¹⁰³⁶) compared the time spent with mandibular opening in healthy controls and OSA patients. Controls spent 88.9% of total sleep time with a narrow mandibular opening (less than 5 mm). In contrast, OSA patient spent 69.3% of the total sleep time with wide mandibular opening (more than 5 mm). Moreover, healthy adults showed no difference in mandibular posture between supine and lateral recumbent positions while OSA patients showed different mandibular opening in supine position during different stages of sleep.

The abnormally low position of the hypermobile mandible results in upper airway disturbances during sleep. Therefore, hypermobility of the temporomandibular joint causes OSA.

Note: Without reference to hypermobility of the temporomandibular joint, Miyamoto 1999 (ibid) proposed a similar description of the events leading to apnoeic episodes.

Microcompetition with a GABP virus results in obesity and hypermobility of the temporomandibular joint, which, in turn, increases susceptibility to OSA. Therefore, obesity should be associated with OSA. As predicted, the association between obesity and OSA is well documented (note that the OSA patients in Ferguson 1997, and Miyamoto 1999, see above, are obese).

e) Summary

Microcompetition explains many otherwise unexplained observations reported in the osteoarthritis literature. The observations include hypermobility of specific joints in obesity, increased osteoarthritis in specific joints in obesity, and the association of obstructive sleep apnea with obesity.

12. Cancer

a) Microcompetition with Foreign DNA

(1) Cell Proliferation

(a) Conceptual Building Blocks

(i) Rb and GABP

The promoter of the retinoblastoma susceptibility (Rb) gene includes an N-box in the (−198, −193) region. A study (Sowa 1997, ibid) constructed several Rb containing plasmids: pX” 1 included the normal Rb promoter, pXRP3m the same segment with a mutated N-box, and RBF-1×4, 4 copies of the Rb N-box. All promoters controlled expression of the luciferase (luc) reporter gene. Cotransfection of hGABPα and hGABPdsβ1 expression plasmids with pXRP1 into the SL2 Drosophila cell line elicited a 10-fold increase in reporter gene activity. Cotransfection with RBF-1×4 showed a 13-fold increase, while cotransfection with pXRP3, carrying the mutated N-box, showed no increase. Based on these observations, Sowa, et al., (1997, ibid) concluded that hGABP has a strong transactivating effect on the Rb gene promoter, suggesting that hGABP is the main transactivator for the core promoter element of the Rb gene.

The following symbolic presentation summarizes the observation in Sowa 1997 (ibid),
↑[p300·GABP·N-box_{Rb}]→↑[mRNA_{Rb}]
Sequence of quantitative events 86: Predicted effect of the p300·GABP·N-box_{Rb} complex on Rb mRNA levels.

(ii) Rb and Cell Proliferation

Let [Cell](t), and Δ[Cell](t) denote number of cells at time t, and the change in cell number at time t due to cell replication and cell death. Then,
[Cell](t)=[Cell](t−1)+Δ[Cell](t−1) Function 37

A time t−1, Δ[Cell](t-1)=0, >0, or <0, will be called steady state, excessive cell proliferation, and excessive cell death, respectively. For simplicity, the t index will be omitted in the following symbolic presentations.

Cancer cells show excessive proliferation (for short, proliferation), or cell replication greater than cell death.

The cell cycle starts with a growth period (G1). Prior to a time in late G1, called R-point, the cell “decides” whether to divide or to exit the cell cycle. An exit results in growth arrest, differentiation, senescence, or death by apoptosis. A decision to divide leads to a series of orderly processes starting with DNA synthesis (S), a second growth period (G2), mitosis (M), and a return to G1. As cells progress through cell cycle, pRb (Rb protein) undergoes a series of phosphorylation events. In G0 and early G1 pRb is primarily unphosphorylated. As cells approach the G1/S boundary; pRb becomes phosphorylated by cyclins D/CDK4 and D/CDK6 kinases with additional phosphorylation by cyclin E/CDK2 kinase in late G1. Phosphorylation is progressive and continuous throughout S-phase and into G2/M. Phosphopeptide analyses demonstrated that pRb is phosphorylated on more than one dozen distinct serine or threonine residues throughout the cell cycle (Sellers 1997¹⁰³⁷), The progressive phosphorylation and dephosphorylation is critical for cell cycle transit.

Observations in several studies (see below) suggest that cells upregulate transcription of Rb to induce growth arrest and differentiation. Growth arrest and differentiation are inversely related to cell proliferation. Therefore, Rb expression is inversely related to cell proliferation.

Symbolically,
↑[mRNA_{Rb}]→↓[Cell]
Sequence of quantitative events 87: Predicted effect of Rb mRNA levels on cell number.

Let un-pRb denote unphosphorylated pRb, hypo-pRb, denote hypo, or underphosphorylated pRb, and hyper-pRb, hyperphosphorylated pRb. Un/hypo-pRb denotes all pRb molecules either un-, or hypophosphorylated. Many observations suggest that accumulation of un/hypo-pRb leads to arrest in G1. For instance, E2F is a transcription factor associated with cell proliferation. Un/hypo-pRb, but not hyper-pRb, binds and inactivates E2F. Other studies reported cell proliferation following introduction of viral oncogenes, such as HPV16 E7, adenovirus E1A, and simian virus 40 (SV40) large T antigen, which selectively bind un/hypo-, but not hyper-pRb. A study (Dou 1998¹⁰³⁸) also showed that transfection of Rb into the human osteogenic sarcoma cells SAOS-2, which lack full-length nuclear pRb protein, increased the number of cells in G0/G1 growth arrest. Moreover, co-transfection of cyclin D2, E, or A increased pRb phosphorylation and released the cells from G0/G1 arrest.

Cell cycle arrest and differentiation requires a certain concentration of un/hypo-pRb. To increase the concentration of un/hypo-pRb, cells can dephosphorylate hyper-pRb, decrease degradation of un/hypo-pRb, or create new un-Rb molecules. The following observations suggest that the third mechanism is important in cell cycle regulation. Specifically, the observations suggest that cells upregulate Rb transcription to induce growth arrest and differentiation.

Murine erythroleukemia (MEL) cells are virus-transformed erythroid precursor cells, which can be induced to differentiate by a variety of chemicals. A study (Coppola 1990¹⁰³⁹) induced MEL cells to differentiate with dimethyl sulfoxide (DMSO) or hexamethylene bisacetamide (HMBA). Expression of globin was used as marker of differentiation. The cells showed 11- and 7-fold increase in Rb mRNA in response to DMSO and HMBA treatment, respectively, with maximum expression on day three of induction (Coppola 1990, ibid, FIG. 1). The increase preceded accumulation of globin mRNA. The peak in Rb mRNA occurred simultaneously with growth arrest and terminal differentiation. Another cell line, S2 myoblasts derived from the C3H10T1/2 mouse embryonic cell line by treatment with 5-azacytidine, was induced to differentiate by depletion of mitogens from the growth medium. Expression of α-actin, a muscle specific gene, was used as marker of differentiation. Seven to twelve hours following feeding with 2% horse serum (i.e. conditions of low mitogen), the cells showed an increase in pRb mRNA. The increase continued over the following 48 hours (Coppola 1990, ibid, FIG. 2) and reached a peak level of an approximately 10-fold increase over untreated cells accompanied by an increase in α-actin expression. The Rb gene is expressed at very low levels in the A20 B cell line and the 300-18 pre-B cell line compared to actin. In three plasmacytoma lines, representing very late stages of B cell differentiation, Rb mRNA was 8-fold higher. These observations are consistent with the above observations on MEL and S2 cells. All cell lines showed an increase in steady-state Rb mRNA in late stages of differentiation, a condition maintained in dividing cells. Based on these observations, Coppola, et al., (1990, ibid) concluded that in all three lineages (erythroid, muscle, and B-cell), differentiation is associated with increased Rb mRNA.

A study (Levine 1998¹⁰⁴⁰) immortalized an enriched epithelial cell population derived from 20-day fetal rat lungs with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of 21 h. At the non-permissive temperature (40° C.), doubling time increased to more than 80 h (Levine 1998, ibid, FIG. 4a). 20-3 cells incubated at the permissive temperature showed almost no Rb mRNA; while at the non-permissive temperature the cells showed a more than 100-fold increase in Rb mRNA (Levine 1998, ibid, FIG. 6b). The increase became significant 24 h after, the temperature up-shift and peaked at 48-72 h (Levine 1998, ibid, FIG. 7a). Terminally differentiated and growth-arrested alveolar type I cells are first observed at days 20-21 of gestation. Prior to this time, the lung shows active growth and cell proliferation. Total RNA was isolated from 17-, and 21-day fetal lungs and assayed for Rb mRNA. The results showed a 2.5-fold increase in Rb mRNA during this period relative to a control gene encoding elongation factor Tu (EFTu).

Another study (Slack 1993¹⁰⁴¹) induced murine PI 9 embryonal carcinoma cells to differentiate into neuroectoderm with retinoic acid (RA). Undifferentiated cells showed very low levels of both Rb mRNA and protein. Twenty-four hours following RA exposure, the cells showed a marked increase in Rb expression with mRNA levels increasing 15-fold by 4-6 days (Slack 1993, ibid, FIG. 2). The post-mitotic neurons developed in RA-treated cultures contained only hypophosphorylated pRb (Slack 1993, ibid, FIG. 7, 8). Note that RAC65, a mutant clone of P19 cells that fails to differentiate, contained a truncated RARα receptor and showed no respond to RA exposure with increased Rb mRNA expression (Slack 1993, ibid, FIG. 3). Based on these observations, Slack, et al., (1993, ibid) concluded that the increased Rb expression associated with cell differentiation appears to result from enhanced Rb transcription.

Another study (Richon 1992, ibid) demonstrated a prolonged G1 period in DS19/Sc9 cells, a MEL cell line, following treatment with hexamethylene bisacetamide (HMBA) (Richon 1992, ibid, FIG. 2A). The cells, which emerged from the prolonged G1, progressed through the cell cycle for at least another two to five generations (cycle time 10 to 12 h), and permanently arrested in G1/G0 expressing characteristics of terminal erythroid differentiation. Over 90% of the DS19/Sc9 cells became irreversibly committed to differentiate by 48 h of culture in the presence of HMBA. Protein extracts prepared from asynchronous cultures induced with HMBA showed a 2-to 3-fold increase in total pRb with no change in the proportions of hypo- or hyper-pRb (Richon 1992, ibid, FIG. 4A). An increase in the level of total pRb was detected as early as 24 h after initiating treatment with HMBA, and pRb levels increased as the number of cells recruited to terminal differentiation increased through 100 h of culture (Richon 1992, ibid, FIG. 4A). HMBA induced an increase in pRb levels in all phases of the cell cycle, while no change was detected in DS19/Sc9 cultured without HMBA. The increase in pRb in cells cultured with HMBA was accompanied by an increase in Rb mRNA, which resulted from a 3.6-fold increase in Rb transcription with no change in mRNA stability. DS19/VCR-C is a vincristine-resistant variant of the parental DS19/Sc9 with an accelerated rate of differentiation. HMBA treatment of DS19/VCR-C showed a more prolonged G1 arrest and a higher percentage of cells committed to terminal differentiation compared to the parental line. During G1 arrest, DS19/VCR-C also showed higher levels of hypo-pRb compared to DS19/Sc9. In HMBA-induced MEL cells, every cell division increased the absolute amount of pRb protein, whereas the degree of phosphorylation continued to fluctuate throughout cell cycle progression. The increase was accompanied by an increase in mRNA levels resulting from an increased rate of transcription.

Based on these observations, Richon 1992 (ibid) proposed a model. An inducer increases Rb transcription resulting in higher hypo- and total-pRb concentration. The increase in hypo-pRb prolongs G1, however, the initial increase in hypo-pRb is most likely insufficient for permanent G1 arrest. Therefore, cells reenter the cell cycle for a few more generations while they continue to accumulate hypo-pRb due to increased Rb transcription. When a critical hypo-pRb concentration is attained, the cells irreversibly commit to terminal differentiation. The model describes the determination of the commitment to differentiate as a stochastic process with progressive increases in the probability of both G1/G0 arrest and differentiation established through successive cell divisions.

Many studies reported a relation between Rb phosphorylation, cell cycle arrest, and differentiation. The studies exploit the differential mobility of un-Rb, hypo-Rb, and hyper-pRb on electrophoretic gels to detect protein phosphorylation or dephosphorylation. Since the studies are focused on the transition between the phosphorylation states, they do not typically report changes in concentration of each form of pRb. Specifically, the studies do not quantify protein levels with densitometry, phosphorimage analysis, or similar means. However, in some cases visual inspection of the gels can provide valuable information. For example, one study (Schwartz B 1998¹⁰⁴²) induced actively growing LS 174T colon cancer cells, which constitutively express pRb, to differentiate with sodium butyrate. Three days following exposure, a lower molecular weight, or unphosphorylated, pRb molecule became visible. After the fourth day of treatment, when significant growth inhibition was achieved, the unphosphorylated species was predominant (Schwartz B 1998, ibid, FIG. 5). A careful inspection of the blots in FIG. 5 suggests that the concentration of hypo-pRb on day 4 (lane 6) is higher than the initial concentration of hyper-pRb (lane 1 and 2). Even if we assume that dephosphorylation of hyper-pRb increases the levels of a hypo-pRb species associated with growth arrest, the differences in total concentration at days 0 and 4 indicate a potential need for increased transcription. However, an increase in mRNA stability or in the rate of translation is also possible.

(b) General Prediction

Consider a cell latently infected with a GABP virus. The cell should show excessive proliferation. Symbolically, [symbol]
[N-boxᵥ]→↓[p300·GABP·N-box_{Rb}]→↓[mRNA_{Rb}]→↑[Cell]
Sequence of quantitative events 88: Predicted effect of foreign N-boxes on cell number.

Microcompetition between the DNA of the GABP virus and Rb for the limiting p300 GABP complex (see chapter on microcompetition, p 128), decreases availability of the complex to the cellular gene, which decreases Rb transcription, and increases cell proliferation.

(c) Observations

(i) Transfection Studies

(a) Note

All studies in the following section used the same experimental design. The objective was to test the effect of a certain gene, viral or cellular, on cell function. To perform the test, the studies inserted a gene of interest into a standard plasmid and then transfected the “test gene” plasmid into certain cells. As controls, the studies used cells transfected with the “empty” vector, that is, the standard plasmid without the gene of interest, and non-transfected, or “wild-type” cells.

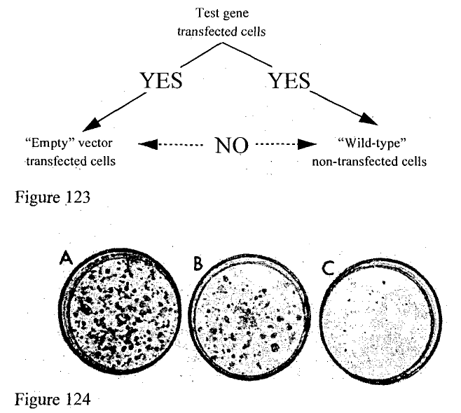

It is interesting that all studies compared, as expected, the cells transfected with the test gene to cells transfected with the “empty” vector, and to non-transfected, wild-type cells. However, no study compared the cells transfected with the “empty” vector to the wild-type cells. See FIG. 123.

(b) Cherington 1988

A study (Cherington 1988¹⁰⁴³) inserted the wild-type early region of SV40, which expresses the SV40 large T antigen into the pZIP-Neo plasmid (the test gene plasmid). The study transfected 3T3-F442A preadipocytes with the test gene or the “empty” pZIP-neo plasmid. Some cells were not transfected (wild-type, WT) (note that, in the paper, the test gene plasmid, and not a non-transfected cell, is labeled “wild-type”). Accumulation of triglyceride, assayed by oil red staining, was used as a marker of differentiation. Seven days post confluence, the study recorded the staining of cells.

pZIP-neo expresses the neomycin-resistance gene under control of the Moloney murine leukemia virus long terminal repeat (LTR) (Cepko 1984¹⁰⁴⁴). The LTR binds GABP (see above). Rb is a GABP stimulated gene (see above). A decrease in Rb expression decreases cellular differentiation. According to microcompetition with foreign DNA, [symbol]
[pZIP-Neo]→↓[p300·GABP·N-cox_{Rb}]→↓[mRNA_{Rb}]→↓[Triglyceride]
Sequence of quantitative events 89: Predicted effect of the “empty” pZip-Neo vector on triglyceride concentration.

Transfection with the “empty” vector, pZIP-neo, should decrease accumulation of triglycerides relative to non-transfected cells. ([Triglyceride] denotes the concentration of triglycerides.) Consider FIG. 124 (based on Cherington 1988, FIG. 4A, B and C). Darker staining indicates increased differentiation.

Transfection with the test gene plasmid, the vector expressing the SV40 large T antigen, decreased differentiation; compare triglyceride staining in C and A. Transfection with the “empty” vector, although less effective than the test gene vector, also decreased differentiation. Compare triglyceride staining in B relative to A and C. The observations are consistent with the predicted effect of the “empty” pZIP-Neo vector on cell differentiation.

(c) Higgins 1996

A study (Higgins 1996¹⁰⁴⁵) transfected murine 3T3-L1 preadipocytes with PVU0, a vector that carries an intact early region of the SV40 genome, which includes the SV40 large and small tumor antigens (the test gene plasmid). The cells were also transfected with HSV-neo, or pZIP-neo, as “empty” controls. Following transfection, the study cultured the cells under differentiation inducing conditions, and measured glycerophosphate dehydrogenase (GPD) activity as a marker of differentiation.

HSV-neo is a plasmid that expresses the neomycin-resistance gene under control of the murine Harvey sarcoma virus long terminal repeat (LTR) (Armelin 1984¹⁰⁴⁶). pZIP-neo expresses the neomycin-resistance gene under control of the Moloney murine leukemia virus (MMLV) long terminal repeat (LTR) (Cepko 1984, ibid). Both the LTRs bind GABP (see above). A decrease in mRNA_{Rb} decreases both cell arrest and differentiation. According to microcompetition with foreign DNA, [symbol]
[HSV-neo] or [symbol] [pZIP-neo]→↓[p300·GABP·N-box_{Rb}]→↓[mRNA_{Rb}]→↓[GPD]
Sequence of quantitative events 90: Predicted effect of the “empty” vectors HSV-neo or pZip-neo on glycerophosphate dehydrogenase (GPD) concentration.

Transfection with the “empty” vectors, HSV-neo or pZIP-neo, should decrease cell differentiation relative to non-transfected cells (WT control) ([GPD] denotes GPD activity.) Table 16 presents the results (Higgins 1996, ibid, Table 1, first four lines).

TABLE 16
Observed GPD activity in WT control, “empty” vector,
and test gene transfected cells.

| | | GPD activity | |
|---|---|---|---|
| | | (U/mg of | |
| Vector | Cell line | protein) | |
| None (WT control) | L1 | 2,063 | 1,599 |
| HSV-neo (“empty” | L1-HNeo | 1,519 | 1,133 |
| vector control A) | | | |
| pZIP-neo (“empty” | L1-ZNeo | 1,155 | 1,123 |
| vector control B) | | | |
| PVU0 (test gene) | L1-PVU0 | 47 | 25 |

| | |
|---|---|
| P value (EV-HSV vs. WT) | 0.118 |
| P value (EV-ZIP vs. WT) | 0.103 |

Transfection with PVU0, which expresses the large and small T antigens, significantly decreased GPD activity. Transfection of the “empty” vectors, HSV-neo and ZIP-neo, although less effective than PVU0, also decreased GPD activity. In a t-test, assuming unequal variances, the p-value for the difference between the HSV-neo vector and no vector is 0.118, and the p-value for the difference between ZIP-neo and no vector is 0.103. Given that the sample includes only two observations, a p-value around 10% for vectors carrying two different LTRs indicates a trend. The observations are consistent with the predicted effect of the “empty” HSV-neo and Zip-Neo vectors on cell differentiation.

(d) Awazu 1998

A study (Awazu 1998¹⁰⁴⁷) transfected HuH-7 human hepatoma cells with pBARB, a plasmid that expresses the Rb gene under the control of the P-actin promoter (hence, the BA RB in the name), and expresses the neomycin-resistance (neo) gene under the control of the simian virus (SV40) promoter. The study also transfected cells with the pSV40-neo plasmid, which only includes the SV40 promoter and the neo gene. Since pSV40-neo does not include the P-actin promoter and Rb gene, the study considered the pSV40-neo plasmid as “empty” and used it as a control. The cells were incubated in IS-RPMI, with or without 5% FBS, and the number of viable cells was counted at the indicated times.

The “empty” vector includes the SV40 promoter that binds p300·GABP. According to microcompetition with foreign DNA, [symbol]
[pSV40-neo]→↓[p300·GABP·N-box_{Rb}]→↓[mRNA_{Rb}]→↑[Cell]
Sequence of quantitative events 91: Predicted effect of the “empty” vector pSV40-neo vector on cell proliferation.

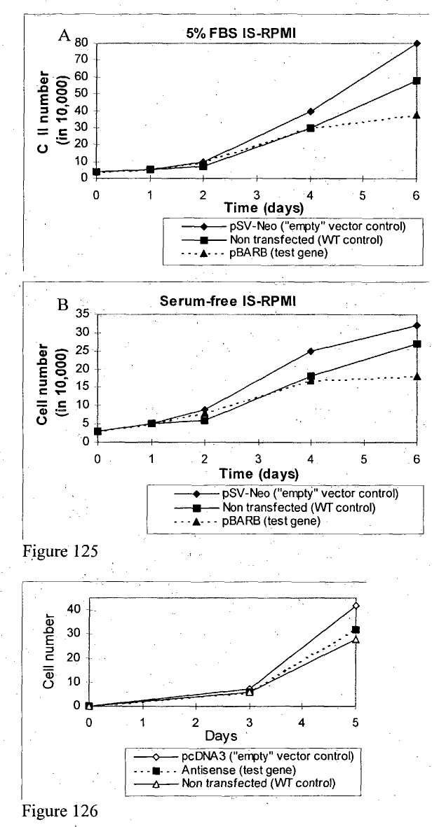

Transfection with the “empty” vector should increase the number of viable cells compared to non-transfected cells (WT), that is, it should induce cell proliferation. FIG. 125 summarizes the results (based on Awazu 1998, ibid, FIG. 2A). The standard deviation (SD) is about the size of the triangular/rectangular symbols.

The observations are consistent with the predicted effect of the “empty” pSV40-neo vector on cell proliferation.

(e) Choi 2001

Another study (Choi 2001¹⁰⁴⁸) stably transfected the human multiple myeloma (MM)-derived cell line ARH with the pcDNA3 plasmid carrying an antisense to the macrophage inflammatory protein 1-α (MIP-1α) (AS-ARH) (the test gene plasmid). As a control, the study transfected other ARH cells with the “empty” pcDNA3 vector (EV-ARH). To measure the effect of the antisense on cell growth, the study cultured 10⁵ non-transfected (WT control), pcDNA3 (“empty” vector control), and MIP-1a antisense (test gene plasmid)transfected ARH cells in six-well plates with RPMI-1640 media containing 10% FBS. At days 3 and 5, the cells were sampled, stained, and counted. The pcDNA3 vector carries the cytomegalovirus (CMV) promoter that binds p300·GABP. According to microcompetition with foreign DNA, [symbol]
[pcDNA3]→↓[p300·GABP·N-box_{Rb}]→↓[mRNA_{Rb}]→↑[Cell]
Sequence of quantitative events 92: Predicted effect of the “empty” vector pcDNA3 on cell number.

Transfection with the “empty” vector should increase the number of viable cells compared to non-transfected cells, that is, it should induce cell proliferation. Consider FIG. 126 Choi 2001, ibid, FIG. 2a).

As expected, after 5 days in culture, the number of cells transfected with the “empty” vector was larger than the number of non-transfected cells. The observations are consistent with the predicted effect of the “empty” pcDNA3 vector on cell proliferation.

(f) Hu 2001

Another study (Hu 2001¹⁰⁴⁹) measured the efficacy and safety of an immunoconjugate (icon) molecule, composed of a mutated mouse factor VII (mfVII) targeting domain, and the Fc effector domain of an IgG1 Ig (mfVII/Fc icon), in the severe combined immunodeficient (SCID) mouse model of human prostatic cancer. First, the study injected the mice s.c. in both rear flanks with the human prostatic cancer line c4-2. The injection resulted in skin tumors.

On days 0, 3, 6, 9, 12, 15, 33, 36, 39, and 42, the study injected into the skin tumor on one flank the pcDNA3.1 (+) vector carrying the icon (four mice), or the “empty” vector (four mice). The tumor on the other flank was left uninjected. The study measured tumor volume in the injected and non-injected flanks.

The pcDNA3.1 (+) vector carries the cytomegalovirus (CMV) promoter that binds p300·GABP. According to microcompetition with foreign DNA, [symbol]
[pcDNA3.1(+)]→↓[p300·GABP·N-box_{Rb}]→↓[mRNA_{Rb}]→↑[Cell]→↑Tumor volume
Sequence of quantitative events 93: Predicted effect of the “empty” pcDNA3.1(+) vector on tumor volume.

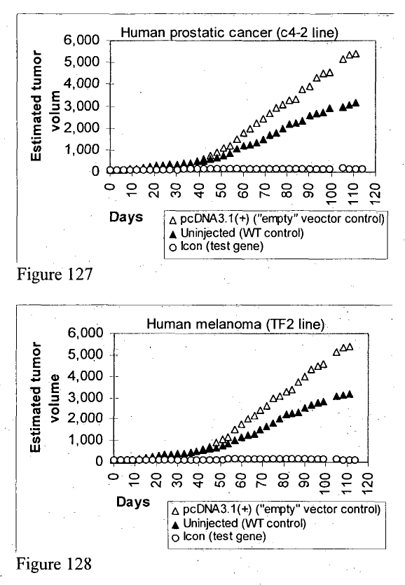

Injection of the “empty” vector transfected cells should increase the volume of the injected tumors compared to uninjected tumors. FIG. 127 presents the results (Hu 2001, ibid, FIG. 3).

As expected, tumors injected with the “empty” vector transfected cells showed higher volumes compared to uninjected tumors.

The experiment was repeated with the human melanoma line TF2 instead of the human prostatic cancer line C4-2. FIG. 128 presents the results (Hu 2001, ibid, FIG. 5).

As expected, tumors injected with the “empty” vector transfected cells showed higher volumes compared to uninjected tumors.

In both experiments, injection of the “empty” vector stimulated tumor growth. Compare (Δ)-tumors injected with the “empty” vector and (▴)-uninjected tumors in the “empty” vector injected mice (WT control). In a t-test, assuming unequal variances, the p-value for the difference between tumors injected with the “empty” vector (Δ) and uninjected tumors (▴), in both experiments, is 0.0265, which is considered statistically significant. The observations are consistent with the predicted effect of the “empty” pcDNA3.1 (+) vector on tumor growth.

(g) Summary

The following table lists some of the materials and methods used in the cited studies.

TABLE 17
Some of the materials and methods used in cited studies.

| | | | | “Empty” |
|---|---|---|---|---|
| | “Empty” | Promoter/ | Cell type/ | vector |
| Study | Vector | enhancer* | tissue | introduction |
| Cherington | pZIP-neo | MMLV | 3T3-F442A | Infection |
| 1988 | | | preadipocytes | with |
| | | | | retrovirus |
| Higgins | HSV-neo | HSV | 3T3-L1 | Calcium |
| 1996 | | | preadipocytes | phosphate |
| | | | | precipitate |
| | | | | transfection |
| | pZIP-neo | MMLV | 3T3-L1 | Calcium |
| | | | preadipocytes | phosphate |
| | | | | precipitate |
| | | | | transfection |
| Awazu | pSV-neo | SV40 | HuH-7 human | Lipofection |
| 1998 | | | hepatoma cells | transfection |
| Hu 2001 | pcDNA3 | CMV | Skin tumors | Injection |
| | | | | into skin |
| Choi 2001 | pcDNA3 | CMV | Human multiple | Lipofectamine |
| | | | myeloma (MM)- | plus |
| | | | derived cells | transfection |

*MMLV- Moloney Murine Leukemia Virus, SV40- Simian Virus No 40, CMV- Cytomegalovirus, HSV- Murine Harvey Sarcoma Virus

Although the cited studies used different materials and methods, such as different plasmids, different transfection methods, different cell types, and different organisms, the observations, in all studies, are consistent with the predicted effect of microcompetition with foreign DNA.

The argument for large sample sizes, randomization, independent verification by different laboratories, etc., is to even out specific peculiarities inherent in any single measurement. The same result under dissimilar conditions is considered reliable. Since the effect of the “empty” vector on cell function was observed under a variety of conditions using dissimilar materials and methods, the effect is, most likely, not an artifact of any specific study, and therefore, reliable. The added reliability increases the level of confidence in the validity of the proposed new concept.

One of the most powerful instruments in the scientific tool bag is the paradigm, the mental model that represents reality (Kuhn 1962¹⁰⁵⁰). However, a paradigm is also a filter for perception. The above studies illustrate the blinding power of a paradigm. The current view holds that viral proteins are the sole mediators of viral effects on the host cell. Such proteins include, for example, the papillomavirus type 16 E6 and E7 oncoproteins, SV40 large T antigen, Epstein-Barr virus BRLF1 protein, and adenovirus E1A. Following the “protein-dependent” assumption, the standard plasmid, which did not express the protein of interest, was called “empty,”⁵ and was used in preparing control cells. As expected, these cells were never compared with the other controls, the non-transfected cells. The viral “protein-dependent” view has such a strong hold on the scientists' mind that even when a “protein-independent” effect on cell function presented itself in the laboratory, the effect was ignored.
⁵ Why did the authors choose the word “empty,” Note that the word empty, like the number zero, has two meanings: a relative and an absolute one. In relative terms, empty means less than full. So one answer might be that the standard plasmid is “emptier” compared to the “fuller” test gene plasmid. However, empty also means “nothing,” and therefore, another reason for the choice might be that the word “empty” also indicates “no effect.” Since the “empty” plasmid had a significant effect on cell function, we chose to mark the word empty with quotation marks throughout the report.

(h) Note on Latent Infections

A latent infection with a GABP virus results in microcompetition between cellular genes and foreign DNA for a limiting factor. However, how can a few viral genomes, the typical viral copy number in latent infections, compete with all genes in the human genome?

To appreciate the potential effect of a few viral genomes, one should think in probabilistic rather than deterministic terms, and in chronic rather than acute disease terms.

(i) Activation Time

Some viruses show permanent, constitutive occupancy of their enhancer regions (Jacque 1996¹⁰⁵¹). In contrast, many cellular genes show promoter occupancy only upon signal stimulation (Garrity 1994¹⁰⁵²). The observation suggests that, at any given moment, a viral enhancer competes with only active cellular promoters. Non-active promoters are irrelevant.

How many cellular promoters are active at certain times is still an open question. However, the answer is certainly a subset of all genes in the genome. If this subset is small enough, a few latent viral genomes can have a sizeable effect.

(ii) GABP Regulated Genes

Viral enhancers do not compete with all genes for all transcription factors. Consider one specific complex: p300·GABP. How many cellular promoters bind this complex? Moreover, how many GABP regulated genes are active at any certain moment? Certainly, only a subset of all genes in the genome. As before, if this subset is small enough, a few latent viral genomes can have a sizeable effect.

(iii) Affinity

DNA binding affinity of gene-specific transcription factors ranges from 10⁻⁸M to 10⁻¹¹M in terms of their equilibrium dissociation constants. This is a 3 order of magnitude difference.

Specific information on the difference in affinity of the p300·GABP complex between viral enhancers and cellular gene promoters is lacking. However, a study (Szymczyna 2000¹⁰⁵³) reports more than 70-fold difference in dissociation constants depending on the polynucleotides in the DNA binding site for other members of the ETS family of transcription factors. If a viral N-box shows a higher affinity for the p300·GABP complex relative to certain cellular genes, this difference in affinity should be considered when evaluating the possible effect of microcompetition.

(iv) Viral Enhancers and Vectors

Many vectors use viral enhancers (for instance, the CMV enhancer). It is reasonable to suggest that the proven effectiveness of these enhancers in transfection studies may result from their superior capacity to microcompete for cellular transcription factors.

(v) Weak Effect

Unlike acute diseases, a chronic disease develops over years, sometimes decades. Hence, one should look for a disruption with a weak effect, an effect that modifies transcription of certain genes ever so slightly.

In the case of infection resulting in acute disease, the average incubation time, or the time between infection and appearance of clinical symptoms, is about 10 days. In cancer or cardiovascular disease, incubation time might be 30-40 years, or 10,950-14,600 days (mid point 12,775). The ratio of 10/12,775=0.08%, or less than 1 in 10,000 indicates the size relation between the rate of progression of chronic vs. acute disease, and the difference in magnitude of the effect in both cases. If chronic disease results from an infection, the effect of such infection should be a four order of magnitude smaller compared to the effect of infections resulting in acute disease. A few latent viral genomes, slightly disrupting transcription, fit such a scale.

(ii) BRCA1

(a) Conceptual Building Blocks

(i) BRCA1 and GABP

The breast cancer associated gene type 1 (BRCA1) promoter includes three N-boxes in the (−200, −178) region. A study (Atlas 2000¹⁰⁵⁴) transfected plasmids with point mutations in the central BRCA1 N-box, alone or in combination with mutations in the other N-boxes, into MCF-7 human breast cells. The mutated plasmids showed a 3-fold decrease in promoter activity. Nuclear extracts from MCF-7 formed a specific complex with the region carrying the N-boxes. Through cross-linking, electrophoretic gel supershift assays, and binding to recombinant GABPαβ, GABP was identified as the primary transcription factor interacting with the N-boxes. Finally, an artificial promoter containing the multimerized N-boxes region was transactivated by cotransfection with GABPα and GABPβ1 in both MCF-7 and T47D breast cells. The observations demonstrate that BRCA1 is a GABP stimulated gene.

The following symbolic presentation summarizes the observations in Atlas 2000 (ibid).
↑[p300·GABP·N-box_{BRCA1}]→↑[mRNA_{BRCA1}]
Sequence of quantitative events 94: Predicted effect of the p300·GABP·N-box_{BRCA1} complex on BRCA1 mRNA levels.

(ii) BRCA1 and Cell Proliferation

Transcriptional or translational inactivation of BRCA1 increases cell proliferation. The relation between BRCA1 and cell proliferation was illustrated in a study (Thompson 1995¹⁰⁵⁵) that treated normal mammary epithelial cells and MCF-7 breast cancer cells with unmodified 18 base oligodeoxyribonucleotides complementary to the BRCA1 translation initiation site. The anti-BRCA1 oligonucleotides, which decreased BRCA1 mRNA by 70-90% compared to control oligonucleotides (Thompson 1995, ibid, FIG. 6), induced accelerated cell proliferation in treated cells (Thompson 1995, ibid, FIG. 4a,c).

Another study (Rao 1996¹⁰⁵⁶) transfected NIH3T3 mouse fibroblasts with a vector expressing BRCA1 antisense RNA. The transfected cells, unlike parental and sense transfectants, showed decreased expression of the endogenous BRCA1 protein, accelerated growth rate, anchorage independent growth, and tumorigenicity in nude mice (Rao 1996, ibid, FIG. 4).

Retroviral transfer of a wild-type BRCA1 gene into breast and ovarian cancer cell lines inhibited growth in vitro. Transfection of wild-type BRCA1 also inhibited development of MCF-7 tumors in nude mice. Peritoneal treatment with retroviral vector expressing wild-type BRCA1 inhibited tumor growth and increased survival among mice with established MCF-7 tumors (Holt 1996¹⁰⁵⁷). A phase I clinical study (Tait 1997¹⁰⁵⁸) extended these observations. The study employed gene transfer of BRCA1 into 12 patients with extensive metastatic cancer. The observations showed stable disease for 4-16 weeks in eight patients, tumor decrease in three patients, and radiographic shrinkage of measurable disease in one patient.

Taken together, these observations suggest an inverse relation between transcription of BRCA1 and cell proliferation, or cell number. Symbolically,
↑[mRNA_{BRCA1}]→↓[Cell]
Sequence of quantitative events 95: Predicted effect of BRCA1 mRNA levels on cell number.

If cell proliferation increases the probability of developing cancer, mutations in BRCA1 gene should result in susceptibility to cancer. As expected many studies show that familial breast cancer results from germline mutations that decrease BRCA1 gene expression.

Symbolically,
↓[mRNA_{BRCA1}]→↑[Cell]→↑[Cancer]
Sequence of quantitative events 96: Predicted effect of BRCA1 mRNA levels on susceptibility to cancer.

(b) Prediction and Observations: BRCA1 in Tumors

An infection with a GABP virus should decreases BRCA1 expression, and increase cell proliferation, and the rate of cancer progression. Symbolically, [symbol]
[N-boxᵥ]→↓[p300·GABP·N-box_{BRCA1}]→↓[mRNA_{BRCA1}]→↑[Cell]→↑[Cancer]
Sequence of quantitative events 97: Predicted effect of foreign N-boxes on susceptibility to cancer, BRCA1 case.

The sequence of quantitative events does not exclude cell proliferation, or cancer, without a decrease in BRCA1 expression, or cancer with a decrease in BRCA1 expression for reasons other than microcompetition with foreign DNA. However, if microcompetition with foreign DNA is a prevalent cause of cancer, many tumors should show decreased BRCA1 transcription otherwise unexplained by traditional methods. Consider the following observations.

Many studies reported decreased BRCA1 transcription in the majority of sporadic breast and ovarian tumors (Russell 2000¹⁰⁵⁹, Rio 1999¹⁰⁶⁰, Rice 1998¹⁰⁶¹, Magdinier 1998¹⁰⁶², Ozcelik 1998¹⁰⁶³, Thompson 1995, ibid). For instance, Magdinier 1999 (ibid) reported a statistically significant decrease in BRCA1 mRNA in 94% of patients tested (Magdinier 1999, ibid, FIG. 2), Rio 1998 (ibid) reported a decrease in BRCA1 mRNA in 100% of the six cell lines tested, and Russell 2000 (ibid) reported a decrease in BRCA1 protein expression in 90% of the tested epithelial ovarian tumors.

Moreover, the cause of decreased transcription in sporadic breast and ovarian cancers is unknown. Two possible causes, somatic mutations and promoter methylation, do not seem to provide an explanation. Somatic mutations of the BRCA1 gene are rare in sporadic breast and ovarian tumors (Russell 2000, ibid, Rio 1999, ibid, Futreal 1994¹⁰⁶⁴, Merajver 1995¹⁰⁶⁵), and methylation of the BRCA1 promoter was demonstrated in only a small percentage of sporadic breast cancer samples (Catteau 1999¹⁰⁶⁶, Magdinier 1998 ibid, Rice 1998, ibid, Dobrovic 1997¹⁰⁶⁷). The majority of breast and ovarian tumors show neither somatic mutations nor BRCA1 promoter methylation.

The frequently observed decrease in BRCA1 transcription in sporadic breast and ovarian cancers is consistent with the predicted effect of microcompetition with foreign DNA on BRCA1 transcription and cancer. Moreover, the lack of somatic mutations or hypermethylation in the majority of these cancer, indicates that microcompetition with foreign DNA might be a prevalent cause of these cancers.

(iii) Fas

(a) Conceptual Building Blocks

(i) Fas and GABP

The promoter of the Fas (Fas, APO-1, CD95) gene includes two N-boxes at regions (−857, −852) and (−833, −828). A study (Li X R 1999¹⁰⁶⁸) transiently transfected Jurkat T-cells with a luciferase reporter gene under control of different length fragments of the Fas promoter. The cells were stimulated for 10 h with anti-CD3 mAb, PMA, or PMA/ionomycin. Deletion of the two N-boxes decreased activation by 50-75%. Mutation of the N-boxes also decreased luciferase activity. Cell stimulation stimulated formation of specific complexes on the N-boxes region. Mutation of the N-boxes and treatment with GABPα- and GABPβ-specific antibodies inhibited formation of these complexes. In complementary experiments, two or four copies of the Fas/GABP site (−863, −820) were inserted into a reporter plasmid carrying the pGL3/promoter. Anti-CD3 mAb, PMA, and PMA/ionomycin treatment stimulated luciferase activity 8-20-fold in Jurkat transfected cells. Mutation of the N-boxes significantly decreased luciferase activity in response to stimulation. These observations demonstrate that Fas is a GABP stimulated gene.

The following symbolic presentation summarizes the observations in Li X R 1999 (ibid).
↑[p300·GABP·N-box_{Fas}]→↑[mRNA_{Fas}]
Sequence of quantitative events 98: Predicted effect of the p300·GABP·N-box_{Fas} complex on Fas mRNA levlels.

(ii) Fas and Cell Death

Programmed cell death, or apoptosis, is the final step in a series of terminal morphological and biochemical events. The Fas antigen is a 48-kDA cell surface receptor homologous to the tumor necrosis factor (TNF) family of transmembrane proteins. Fas binding by the Fas ligand, or by antibodies, triggers rapid cell apoptosis.

Fas induced apoptosis was initially identified in the immune system where ligation of Fas induced apoptosis in activated T-cells, B cells, and natural killer cells. In addition, Fas was identified in many epithelial cells. Although the role of Fas in non-lymphoid tissues is not completely understood, maintenance of normal cell turnover and the removal of potentially oncogenic cells have been suggested. Consider, for example, the epithelial layer of colonic mucosa. These cells show a rapid rate of cell turnover accompanied by high levels of Fas expression. It is conceivable that Fas regulates the high rate of colonocyte removal.

Excessive cell death decreases cell number. Since Fas binding by Fas ligand, or by antibodies, triggers rapid cell apoptosis, an increase in Fas concentration increases the probability of apoptosis, which, in turn, decreases cell number. Symbolically,
↑[mRNA_{Fas}]→↓[Cell]
Sequence of quantitative events 99: Predicted effect of Fas mRNA on cell number.

If cell proliferation increases the probability of developing cancer, mutations in the Fas gene should result in susceptibility to cancer. As expected, Davidson 1998¹⁰⁶⁹ reported an association between germline mutations in the Fas gene and the spontaneous development of plasmacytoid tumors in lpr mice. Drappa 1996 ¹⁰⁷⁰ reported an association between such mutations and neoplasms in two autoimmune lymphoproliferative syndrome (ALPS) patients. Symbolically,
↓[mRNA_{Fas}]→↑[Cell]→↑[Cancer]
Sequence of quantitative events 100: Predicted effect of Fas mRNA on susceptibility to cancer.

(b) Predictions and Observations: Fas in Tumors

An infection with a GABP virus should decrease Fas expression, increase cell proliferation, and increase the rate of cancer progression. Symbolically, [symbol]
[N-boxᵥ]→↓[p300·GABP·N-box_{Fas}]→↓[mRNA_{Fas}]→↑[Cell]→↑[Cancer]
Sequence of quantitative events 101: Predicted effect of foreign N-boxes on susceptibility to cancer, Fas case.

If microcompetition with foreign DNA is a prevalent cause of cancer, many tumors should show decreased Fas transcription unexplained by traditional methods. As expected, several studies detect a progressive decrease in Fas expression in many cancers. Keane 1996¹⁰⁷¹ showed decreased Fas expression in breast carcinomas, Gratas 1998¹⁰⁷² in esophageal carcinomas, Strand 1996¹⁰⁷³ in hepatocellular carcinomas, Moller 1994¹⁰⁷⁴ in colon carcinomas, and Leithauser 1993¹⁰⁷⁵ in lung carcinomas. Moreover, decreased transcription is the cause of the observed decrease in Fas expression in cancer. For instance, Das 2000¹⁰⁷⁶ showed decreased Fas transcription in ovarian, cervical, and endometrial carcinoma tissues. The study also showed decreased Fas transcription in four ovarian, and three cervical carcinoma cell lines. Butler 1998¹⁰⁷⁷ showed decreased Fas transcription in colon tumors, and Keane 1996 (ibid) showed decreased Fas mRNA levels in six out of seven breast cancer cell lines.

As with BRCA1, the cause of decreased Fas transcription is unknown. The two possible causes, somatic mutations and promoter methylation, fail to explain the observed decrease in Fas transcription. Allelic loss or somatic mutations of the Fas gene are rare (Bertoni 2000¹⁰⁷⁸, Lee 1999A¹⁰⁷⁹, Lee 1999¹⁰⁸⁰, Shin 1999¹⁰⁸¹, Butler 1998, ibid), and no methylation was observed in the Fas promoter (Butler 2000¹⁰⁸²) The majority of carcinomas show no somatic mutations or Fas promoter methylation.

The frequently observed decrease in Fas transcription in various cancers is consistent with the predicted effect of microcompetition with foreign DNA on Fas transcription and cancer. Moreover, the lack of somatic mutations or promoter methylation, indicates that microcompetition with foreign DNA might be a prevalent cause of cancer.

Note that the studies with Fas reported a progressive decrease in Fas expression in many cancers. Virus replication under conditions of non-latent infection can result in a progressive increase in viral DNA, which progressively decreases mRNA_{Fas}. The prevalent progressive loss of Fas expression is, therefore, consistent with the predicted effect of microcompetition with foreign DNA on Fas transcription and cancer.

(d) Summary

The specifications report the discovery of microcompetition, a new biological mechanism, and show that disruption of this mechanism decreases cell differentiation and increases cell proliferation. Consider the discovery of the double helix. According to Watson and Crick (1953¹⁰⁸³): “It has not escaped our notice that the specific pairing we have postulated immediately suggests a possible copying mechanism for the genetic material.” The significance of the Watson and Crick discovery was not the description of the molecular structure of DNA, but the hinted mechanism for human inheritance.

When is the discovery of a new mechanism exciting? When the new mechanism can be used to answer important questions that resist conventional means. In the case of the double helix, inheritance was considered, at the time, one of the most intriguing and difficult questions in biology. The above sections presented one of today's important questions: Why is transcription of many wild-type genes decreased, or increased, in chronic disease, and specifically, in cancer?. The discovery of the microcompetition mechanism provides an answer to this question.

(2) Metastasis

Metastasis involves migration of cancer cells from one tissue to another. Assume the model of cell motility (see chapter on cell motility, p 145) applies to cancer cell migration.

(a) Prediction

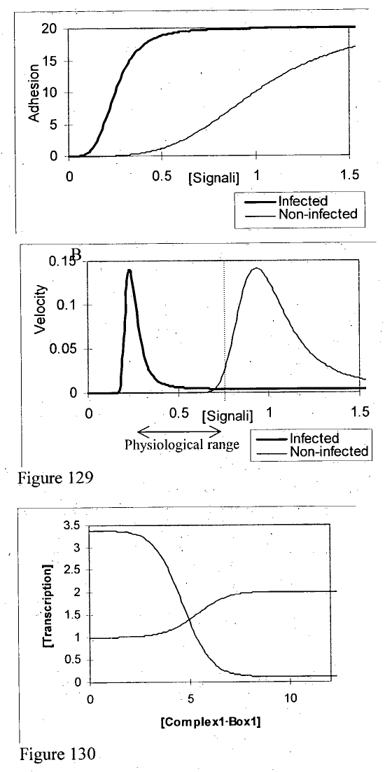

Consider signal; with a biological range of intensities [0,0.5]. Consider a cell with low sensitivity to signal; at that range, that is, an increase in intensity from 0 to 0.5, hardly increases adhesion, and therefore, produces no motility. Consider FIG. 129.

An infection with a GABP virus increases expression of a propulsion gene, say TF. The increase in TF shifts up the adhesion curve and increases the skewness of the velocity curve. As a result, in the physiological range, an infected cell shows cell motility, or metastasis.

Symbolically, [symbol]
[N-boxᵥ]_{cell}→↓[p300·GABP·N-box_{TF}]→↑[TF_{mRNA}]→↑Adhesion curve→↑Skewness of V curve→↑TotalD_{cell}→↑Metastasis
Sequence of quantitative events 102: Predicted effect of foreign N-boxes on metastasis.

The increase in number of viral N-boxes increases skewness of the TF propelled velocity curve, and increases the total distance traveled by the infected cell. Consider the following observations.

(b) Observations: TF and Metastasis

Several studies reported increased TF expression in metastatic tumors (Ohta 2002¹⁰⁸⁴ in prostate cancer, Guan 2002¹⁰⁸⁵ in glioma, Nakasaki 2002¹⁰⁸⁶ in colorectal cancer, Sawada 1999¹⁰⁸⁷ in non-small-cell lung cancers, Shigemori 1998¹⁰⁸⁸ in colorectal cancer, Mueller 1992¹⁰⁸⁹ in melanoma, Adamson 1993¹⁰⁹⁰ in prostate cancer, Kataoka 1997¹⁰⁹¹ in colorectal carcinoma cell lines and metastatic sublines to the liver, Sturm 1992¹⁰⁹² in breast cancer, Hu 1994¹⁰⁹³ in a variety of cancer cell lines). Moreover, TF expression directly correlated with tumor aggressiveness (see also recent reviews, Lee 2002¹⁰⁹⁹, Sampson 2002¹⁰⁹⁵, Gale 2001¹⁰⁹⁶, Rickles 2001¹⁰⁹⁷, Lwaleed 2001¹⁰⁹⁸, Ruf 2000¹⁰⁹⁹, and Schwartz J D 1998¹¹⁰⁰).

To examine the effect of TF on cell migration, a study (Kakkar 1999¹¹¹⁰) cloned the full-length TF gene into the pcDNA3 plasmid, in sense and antisense orientation, and used the plasmids to transfect MIA PaCa-2 human pancreatic adenocarcinoma cells. The study, then, measured TF expression and tumor cell invasion in vitro. Sense transfected cells showed higher TF cell content, and procoagulant activity compared to antisense transfected and wild-type cells (p=0.001 and p=0.008, respectively). Sense transfected cells also showed increased cell invasion compared to antisense transfected and wild-type cells (p 0.001). Based on these observations, Kakkar, et al., (1999, ibid) concluded: “Expression of TF enhances in vitro invasion.”

Another study (Bromberg 1995¹¹⁰²) used retroviral-mediated transfection of a nonmetastatic parental line to generate two matched sets of cloned human melanoma lines expressing different levels of human TF expression. The study injected the tumor cells into the tail vein of severe combined immunodeficiency (SCID) mice, and examined the lungs after 10-11 weeks. The results showed metastatic tumors in 86% of the mice injected with tumor cells expressing high levels of TF, and 5% of the mice injected with the cells expressing low levels of TF. Based on these results, Bromberg, et al., (1995, ibid) concluded: “high TF level promotes metastasis of human melanoma in the SCID mouse model.” A subsequent study (Song 2002¹¹⁰³) reports similar observations (see note below).

These observations are consistent with the predicted effect of microcompetition with foreign DNA on TF expression and metastasis.

Note that the original objective in Song 2002 (ibid) was to examine “whether TF activates an intracellular signaling pathway in human melanoma cells that results in altered gene expression and enhanced metastatic potential.” To that end, the study infected two clones derived from the human melanoma line YU-SIT1, which expresses a low level of TF and is weakly metastatic in SCID mice, with the retroviral vector LXSN that either contained (T2) or did not contain (L8) a TF cDNA insert. Consistent with the observations in Bromberg 1995 (ibid), T2 showed higher TF expression and stronger metastasis in SCID mice compared to L8 cells. To test for altered gene expression, the study screened cDNA libraries prepared from the RNA of the T2 and L8 clones. The results showed that T2 included cDNA of the mouse VL30 retrotransposable element (mVL30 retroelement), while L8 did not. The mouse VL30 retroelement is, most likely, a mutated non-infectious descendent of an infectious retrovirus. To test the effect of mVL30 on metastasis, the study generated 12 clones by infecting the parental line YU-SIT1 with LXSN only or LXSN that contained the TF insert. Testing for mVL30 showed presence of mVL30 RNA and cDNA in four of the eight high TF clones, and two of the four low TF clones. The study then compared the metastatic potential of the twelve clones in SCID mice by injecting the melanoma cells and counting the number of lung tumors 10-11 weeks later. The results showed an average of 2.1 lung tumors per mouse in the high TF clones without mVL30 and 26.7 lung tumors per mouse in the high TF clones with mVL30 RNA and cDNA. In clones expressing high level of TF, presence of mVL30 RNA and cDNA was associated with an increase in tumor metastasis.

The question is how did mVL30 increase tumor metastasis? One possibility is that mVL30 cDNA integration into the cell genome disrupted regulation or function of a key gene involved in oncogenesis. However, the study found that mVL30 cDNA integrated at a different site in every clone (Song 2002, ibid, FIG. 4), and concluded that the metastatic effect of mVL30 is, most likely, not related to the integration site. Another possibility it that metastasis is dependent on mVL30 RNA. However, according to Song, et al., (2002, ibid): “A role for the mVL30-1 RNA in metastasis and possibly other cell functions is an unexpected finding, because the RNA appears to lack significant coding potential for a functional protein.” Therefore, “The metastatic effect might be mediated directly by noncoding mVL30-1 RNA.” But how?

The mVL30 genome includes a 5′ and 3′ long terminal repeat (LTR) that functions as an enhancer of transcription (Pribnow 1996¹¹⁰⁴, Rodland 1993¹¹⁰⁵, Rotman 1986¹¹⁰⁶) Assume the mVL30 enhancer microcompetes with TF for a limiting complex that suppresses TF transcription, possibly, GABP. Then, an increase in mVL30 RNA is associated with an increase in binding of the limiting complex to the mVL30 LTR, decreased binding to TF, increased TF expression, and increased metastasis. Assume mVL30 binds GABP, then, [symbol]
[N-box_{mVL30}]_{cell}→↓[p300·GABP·N-box_{TF}]→↑[TF_{mRNA}]→↑Adhesioin curve→↑Skewness of V curve→↑TotalD_{cell}→↑Metastasis
Sequence of quantitative events 103: Predicted effect ofthe mVL30 genome on metastasis.

Song 2002 (ibid) does not report the expression levels of TF in mVL30 vs. non-mVL30 cells. However, according to the sequence of quantitative event, mVL30 cells should show higher TF expression.

(3) Viral Genomes in Tumors

Microcompetition with foreign DNA is only one disruption that can lead to cancer (see next section for other disruptions). However, frequent detection of viral genomes in human tumors suggests that microcompetition with foreign DNA might be a prevalent cause of cancer. Consider the following table.

TABLE 18
Viral genomes in human tumors.

| Virus | Cancer |
|---|---|
| Epstein-Bar virus (EBV) | Burkitt's lymphoma (BL) |
| | Nasopharyngeal carcinoma (NPC) |
| | Hodgkin's disease |
| | Some T-cell lymphomas |
| | Polymorphic B cell lymphomas |
| | B-cell lymphoproliferation in |
| | immunosuppressed individuals |
| | Breast cancer |
| SV40 | Brain tumors |
| | Osteosarcomas |
| | Mesotheliomas |
| Human T-cell lymphotrophic | Adult T-cell leukemia |
| virus - I (HTLV-I) | |
| Human papillomavirus (HPV) | Anogenital cancers |
| | Skin cancers |
| | Oral cancers |
| Hepatitis B virus (HBV) | Hepatocellular carcinoma |
| Hepatitis C virus (HCV) | Hepatocellular carcinoma |
| Human herpes virus 8 | Kaposi's sarcoma, |
| (HHV8, KSHV) | |
| | Body cavity lymphoma |

See also recent reviews on human tumor viruses by Butel 2000 (ibid), zur Hausen 1999-I¹¹⁰⁷, Hoppe-Seyler 1999¹¹⁰⁸. On EBV and breast cancer, see Bonnet 1999¹¹⁰⁹, Labrecque 1995¹¹¹⁰, and a recent editorial (Magrath 1999¹¹¹¹).

EBV, SV40, and HTLV-I are GABP viruses. The repeated detection of GABP viral DNA in many tumors is consistent with the suggested relation between GABP viruses and cancer.

An interesting observation in some of these studies is the detection of viral DNA without the expression of viral proteins. Consider, for example, the studies on EBV in breast cancer, which reported undetectable EBER expression in many cases positive for EBV DNA by in situ PCR (Bonnet 1999 (ibid) ^{and} Labrecque 1995 (ibid), see also discussion on this observation and more examples in the editorial by Magrath and Bhatia (1999, ibid). Moreover, in many studies, the detected viral DNA was replication defective. These observations are inconsistent with the current protein-dependent view of viral effects, yet are consistent with microcompetition with foreign DNA, a protein-independent approach.

b) Other Disruptions in p300 Allocation

(1) Allocation Model

Consider Rb as example. Rb is a GABP stimulated gene. Therefore,

$$\begin{matrix}[ {mRNA}_{Rb} ] = & {f}_{A \text{-} T} ( [ {DNA}_{Rb} ] , & [ {DNA}_{v \text{-} GABP} ] , & {Affinity}_{v / Rb} , & [ {GABPkinase}^{phos} ] , & OS ) \\ & ( + ) & ( - ) & ( - ) & ( + ) & ( - )\end{matrix} \qquad Function 38$$

See the chapter on signaling and allocation, p 330, for the meaning of the symbols and a discussion on the f_{A-T} function.

The following f_{A-CN} function relates microcompetition with foreign DNA, phosphorylation of GABP kinases, and oxidative stress to cell number. The subscript A-CN indicates a relation between p300 allocation and cell number.

$$\begin{matrix}[ Cell ] = & {f}_{A \text{-} CN} ( [ {DNA}_{Rb} ] , & [ {DNA}_{v \text{-} GABP} ] , & {Affinity}_{v / Rb} , & [ {GABPkinase}^{phos} ] , & OS ) \\ & ( - ) & ( + ) & ( + ) & ( - ) & ( + )\end{matrix} \qquad Function 39$$

Since mRNA_{Rb} expression decreases cell number, the signs under the independent variables in f_{A-CN} are reversed relative to f_{A-T}, for instance, the signs under [DNA_{Rb}] in f_{A-CN} and in f_{A-T} are (−) and (+), respectively.

Similar functions can be formulated for BRCA1 and Fas. A f_{A-CN} function will be called the “allocation model of cell proliferation.”

A system in stable equilibrium is a system that always returns to the equilibrium. Let a healthy biological system be identified with a certain stable equilibrium. Any exogenous event that produces a new stable equilibrium will be called “disruption.” Any stable equilibrium different from the healthy system equilibrium will be called “chronic disease.” Infection with a GABP virus is an exogenous event, or disruption. In contrast, phosphorylation/dephosphorylation of GABP kinases and oxidation/decrease are events endogenous to the biological system, and, therefore, by themselves, are not disruptions. Nevertheless, exogenous events, such as a mutation in the ERK agent transforming growth factor-β (TGFβ), which excessively decreases GABP kinase phosphorylation, or a sustained exposure to nicotine, which excessively increases oxidative stress, are disruptions. The excessive decrease in GABP kinase phosphorylation and excessive increase in oxidative stress, unlike normal variations, produce new p300 allocations and disease.

According to the “allocation model of cell proliferation,” an infection with a GABP virus is a disruption of p300 allocation. Excessive decrease in GABP kinase phosphorylation, and excessive oxidative stress are also disruptions of p300 allocations. These disruptions increase cell proliferation, and increase the probability of developing cancer.

(2) GABP Kinase Phosphorylation

Transforming growth factor-β (TGFβ) is an ERK agent (see chapter on signaling and allocation, p 330). Consider Rb as examples. According to the “allocation model of cell proliferation,” [symbol]
[TGFβ receptor type II]→↓[ERK^{phos}]→↓[GABP^{phos}]→↓[p300·GABP·N-box_{Rb}]→↓[mRNA_{Rb}]→↑[Cell]→↑[Cancer]
Sequence of quantitative events 104: Predicted effect of TGFβ receptor type II on susceptibility to cancer.

A mutation in the TGFβ receptor type II gene that results in receptor deficiency decreases ERK phosphorylation, GABP phosphorylation, GABP binding to p300, Rb transcription, and should increase cell proliferation, and the probability of developing cancer.

A study (Park 1994¹¹¹²) observed genetic changes in the TGFβ-type II receptor gene in human gastric cancer cell lines resistant to the growth inhibitory effect of TGFβ. Some of the TGFβ resistant cells showed truncated or no detectable TGFβ type II receptor mRNA and protein. Two other studies (Myeroff 1995¹¹¹³, Markowitz 1995¹¹¹⁴) observed mutations in the TGFβ receptor type II gene, decreased concentration of the receptor transcript, and decreased cell surface expression of the receptor in human colon cell lines and gastric cancers.

The observations in Park 1994 (ibid), Myeroff 1995 (ibid), and Markowitz 1995 (ibid) are consistent with the predicted effect of mutations in the TGFβ-type II receptor on cancer.

(3) Oxidative Stress

According to the “allocation model of cell proliferation,” oxidative stress decreases binding of GABP to the N-box, decreases transcription of GABP stimulated genes, and increases cell proliferation and the probability of developing cancer.

Oxidative stress also increases replication of some GABP viruses; see, for instance, the stimulatory effect of oxidative stress on cytomegalovirus (CMV) (Vossen 1997¹¹¹⁵, Scholz 1996¹¹¹⁶), Epstein-Barr virus (EBV) (Ranjan 1998¹¹¹⁷, Nakamura 1999¹¹¹⁸), and HIV (Allard 1998A¹¹¹⁹, Allard 1998B¹¹²⁰). According to the proposed model, infection with these GABP viruses amplifies the effect of oxidative stress on cellular GABP regulated gene transcription. In these cells, oxidative stress stimulates cell proliferation through the inhibition of GABP binding to the N-box and by accentuating microcompetition with foreign DNA. Consider Rb as example, then,
[OS]→↓[p300·GABP·N-box_{Rb}]→↓[mRNA_{Rb}]→↑[Cell]→↑[Cancer]
Sequence of quantitative events 105: Predicted effect of oxidative stress (OS) on susceptibility to cancer.

As predicted, many oxidative stress inducers are known carcinogens, for instance, nicotine (Helen 2000¹¹²¹, Yildiz 1999¹¹²², Yildiz 1998¹¹²³), and asbestos (Afaq 2000¹¹²⁴, Abidi 1999¹¹²⁵, Liu 2000¹¹²⁶, Marczynski 2000A¹¹²⁷, Marczynski 2000B¹¹²⁸, Fisher 2000¹¹²⁹, Brown 2000¹¹³⁰); The effect of these carcinogens on GABP binding and viral replication, might be the main reason for their carcinogenic capacity.

c) Treatment

Treatment is defined an exogenous event that decreases microcompetition with foreign DNA, increases GABP kinase phosphorylation, or decreases oxidative stress. Treatment can be viewed as a “reversed disruption.” The following section presents predicted and observed effects of some treatments on cell proliferation and differentiation. Additional treatments are discussed in the chapter on treatment, p 444.

(1) GABP Kinase Agents

(a) MEK1 and Differentiation

A study (Lessor 1998, ibid) transiently transfected AU565 breast carcinoma cells with a constitutively active MEK1 mutant (pMEK1) or control vector. Expression of the constitutively active MEK1 significantly increased ERK activity (Lessor 1998, FIG. 6A, B). Oil Red O staining was used as a measure of cell differentiation. Consider Rb as example. According to the “allocation model of cell proliferation,” [symbol]
[pMEK1]→↑[ERK^{phos}]→↑[GABP^{phos}]→↑[p300·GABP·N-box_{Rb}]→↑[mRNA_{Rb}]→↑Differentiation/Oil Red O
Sequence of quantitative events 106: Predicted effect of a vector expressing a constitutively active MEK1 mutant (pMEK1) on cell differentiation.

pMEK1 increases formation of the p300·GABP·N-box_{Rb} complex, increases Rb transcription, and cell differentiation as indicated by Oil Red O staining. (“Differentiation/Oil Red O” denotes differentiation as indicated by Oil Red 0 staining.)

As expected, transfection with pMEK1 increased the number of cells showing positive Oil Red O staining (53.6%) compared to transfection with a control vector (20.8%). Based on these observations, Lessor, et al., (1998, ibid) concluded that constitutive activation of the MEK/ERK pathway in AU565 cells is sufficient to mediate differentiation.

The observations in Lessor 1998 (ibid) are consistent with the predicted effect of the ERK agent MEK1 on cell differentiation.

Note: What if the AU565 breast cancer cells harbor a latent GABP virus? Latency means controlled viral replication, and, therefore, limited concentration of viral N-boxes in a cell. As an ERK agent, pMEK1 should stimulate formation of p300·GABP complexes on both viral and cellular N-boxes. However, because the infection is latent, the virus resists the stimulating effect of pMEK1, and prevents the increase in [p300·GABP·N-boxᵥ]. Otherwise, if the pMEK1 stimulating effect on [p300·GABP·N-boxᵥ] is greater than on [p300·GABP·N-box_{Rb}], transfection with pMEK1 would have decreased differentiation. To summarize, pMEK1 stimulates differentiation, even in cells that harbor a GABP virus, as long as the virus continues to replicate under latency conditions (see related note in chapter on signal resistance, p 340).

(b) HRGβ1 and Proliferation/Differentiation

Lessor 1998 (ibid) also treated the AU565 breast carcinoma cells with 10 ng/ml heregulin β1 (HRGβ1) for 7 days. HRGβ1 increased ERK activity 4-fold in as short as 10 min. The initial increase dropped to control levels by 15 min, however, following the drop, a second sustained increase in activity was observed for 105 min (Lessor 1998, ibid, FIG. 1). Following HRGβ1 treatment, the study counted the cells and examined cell differentiation. According to the “allocation model of cell proliferation,” as an ERK agent, HRGβ1 should decrease cell number and increase differentiation. Symbolically, [symbol]
[HRGβ1]→↑[ERK^{phos}]→↑[GABP^{phos}]→↑[p300·GABP·N-box_{Rb}]→↑[mRNA_{Rb}]→↑[Cell] and ↑Differentiation
Sequence of quantitative events 107: Predicted effect of heregulin β1 (HRGβ1) on cell number and differentiation.

As expected, the results showed a 56% decrease in number of cells following treatment with HRGβ1 compared to untreated controls (Lessor 1998, ibid, FIG. 4). Moreover, addition of 0-10 μM PD98059, a specific MEK inhibitor, dose-dependently reversed the HRGβ1-induced increase in cell growth arrest (Lessor 1998, ibid, FIG. 4). Also, as expected, treatment with HRGb1 increased cell differentiation, and pretreatment with PD98059 dose-dependently inhibited the HRGβ1-induced increase in cell differentiation(Lessor 1998, ibid, FIG. 5). Based on these observations, Lessor, et al., (1998, ibid) concluded that sustained⁶ activation of the MEK/ERK pathway is both essential and sufficient for HRGβ1-induced differentiation of AU565 cells.
6Exposure to low doses of HRG I (0.01 ng/ml) induced a 7-fold transient 5 min peak in ERK activation, which dropped to control levels by 90 min. This dose showed no sustained activation (Lessor 1998, ibid, FIG. 1). The 0.01 ng/ml HRGβ1 treatment resulted in cell proliferation.

The observations in Lessor 1998 (ibid) are consistent with the predicted effect of the ERK agent HRGβ1 on cell proliferation and differentiation.

(c) TPA and Proliferation/Differentiation

A study (He H 1999¹¹³¹) treated ML-1 human myeloblastic leukemic cells with 0.3 ng/ml phorbol ester (TPA, PMA). The treatment increased ERK activity 6- and 4-fold at 1 and 3 h, respectively, thereafter decreasing to subbasal levels (He H 1999, ibid, FIG. 1A). According to “allocation model of cell proliferation,” TPA treatment should decrease ML-1 cell proliferation and increase differentiation. Symbolically, [symbol]
[TPA]→↑[ERK^{phos}]→↑[GABP^{phos}]→↑[p300·GABP·N-box_{Rb}]→↑[mRNA_{Rb}]→↑[Cell] and ↑Differentiation
Sequence of quantitative events 108: Predicted effect of phrbol ester (TPA) on cell number and differentiation.

As predicted, 3-day treatment with 0.3 ng/ml TPA, followed by additional 3 days in culture after removal of TPA, induced ML-1 cells to cease proliferation and display morphological features typical of monocytes/macrophages (He H 1999, ibid, FIG. 6c). Exposure to PD98059, the MEK inhibitor, led to a 2- and 10-fold decrease in TPA-stimulated ERK2 activity at 1 and 3 h, respectively (He H 1999, ibid, FIG. 3). Cells treated simultaneously with 10 μM PD98059 and 0.3 ng/ml TPA continued to proliferate and exhibited the morphology of undifferentiated cells (He H 1999, ibid, FIG. 6A, D). Based on these observations, He H, et al., (1999, ibid) concluded that activation of the MEK/ERK signaling pathway is necessary for TPA-induced mononuclear cell differentiation.

The observations in He H 1999 (ibid) are consistent with the predicted effect of the ERK agent TPA on cell proliferation and differentiation.

(d) TGFβ1 and Proliferation

A study (Levine 1998, ibid) immortalized an enriched epithelial cell population from fetal rat lungs at 20-days of gestation with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of approximately 21 h. At the non-permissive temperature (40° C.), doubling time increased to more than 80 h (Levine 1998, ibid, FIG. 4a). The labeling index is a function of [³H]thymidine incorporation into DNA, and, therefore, correlates with cell proliferation. According to the “allocation model of cell proliferation,” treatment with the ERK agent transforming growth factor-β1 (TGFβ1) should decrease cell proliferation as indicated by a decreased labeling index. Symbolically, [symbol]
[TGFβ1]→↑[ERK^{phos}]→↑[GABP^{phos}]+43N ↑[p300·GABP·N-box_{Rb}]→↑[mRNA_{Rb}]→↑[Cell]/labeling index
Sequence of quantitative events 109: Predicted effect of TGFβ1 on cell number.

The symbol [Cell]/labeling index denotes cell number as indicated by the labeling index.

As predicted, the results showed a decrease in the labeling index from 95% to 80% following 72-hour treatment of 20-3 cells with 5 ng/ml TGFβ I at the permissive temperature, and from 20% to less than 5% at the non-permissive temperature (Levine 1998, ibid, FIGS. 5a and 5c). Treated cells cultured at the non-permissive temperature for 72 h and then shifted to the permissive temperature for additional 24 h showed a decrease in the labeling index from 60% to below 10%. Treatment with TGFβ1 decreased proliferation of the epithelial cells at both the permissive and non-permissive temperatures. The study also measured Rb mRNA. At the permissive temperature, mRNA_{Rb} was barely detectable. At the non-permissive temperature, mRNA_{Rb} was increased 100-fold (Levine 1998, ibid, FIG. 6b). The study does not report the effect of TGFβ1 on Rb expression.

The observations in Levine 1998 (ibid) are consistent with the predicted effect of the ERK agent TGFβ1 on cell proliferation

d) Summary

Microcompetition with foreign DNA explains many otherwise unexplained observations reported in the cancer literature. The observations include detection of protein free, replication defective viral genomes in many tumors, progressive decrease in BRCA1 and Fas transcription in many tumors, the carcinogenic effect of epigenetic carcinogens, and the anti-carcinogenic effect of GABP kinase agents.

13. Technical note: ΣS

(1) Signaling and S-Shaped Transcription

(a) S-Shaped Transcription

(i) Model

Assume a transcription complex, Complex₁, regulates transcription of a gene, G, by binding the DNA sequence, Box₁. [Complexᵢ·Boxᵢ] denotes Complexᵢ·Boxᵢ concentration, or the probability of detecting the complex bound to its box. Let Complexᵢ and Boxᵢ, i=2 . . . N, denote all other complexes and boxes, respectively, regulating G transcription. Assume that, for i=2 . . . N, [Complexᵢ·Boxᵢ] is fixed, that is, there is no change in binding of other transcription complexes. Let [Transcription]_{G} denote the rate of G transcription, and f_{G-Complex} the function relating [Transcription]_{G} and [Complex₁·Box₁], that is,
[Transcription]_{G}=f_{G-Complex}_{₁}([Complex₁·Box₁]) Function 40

According to the ΣS model of transcription, for every transcription complex, Complex₁, f_{G-Complex1} can be represented by either an increasing or decreasing S-shaped curve (hence, the S in the name of the model. The Σ is explained below). Consider the following illustration.

If Complex₁ is a stimulator of G transcription, f_{G-Complex1} can be represented by an increasing S-shaped curve. If Complex₁ is a suppressor, f_{G-Complex1} can be represented by a decreasing S-shaped curve. p300·GABP is a transcription complex. According to the ΣS model of transcription, for every GABP suppressed gene, G, f_{G-p300·GABP} can be represented by a decreasing S-shaped curve. Consider the following observations.

(ii) Predictions

(a) Androgen Receptor (AR) Gene

Observations in some studies suggest that GABP suppresses AR transcription (see chapter on alopecia, p 404). According to the ΣS model of transcription, f_{AR-p300·GABP} can be represented by a decreasing S-shaped curve:

$$\begin{matrix}{[ Transcription ]}_{AR} = & {f}_{AR \text{-} p300 \cdot GABP} ( [ p300 \cdot GABP \cdot N \text{-} {box}_{AR} ] ) \\ & ( - )\end{matrix} \qquad Function 41$$

HeLa cells show very low expression of endogenous AR, while LNCaP cells show high expression. Assume variations in GABP suppression as the cause of the difference in baseline AR expression, that is, assume greater [p300·GABP·N-box_{AR}] in HeLa compared to LNCaP cells.

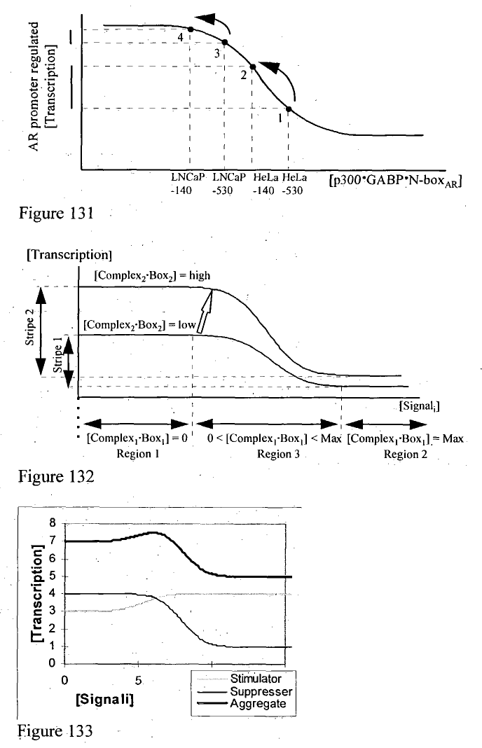

Consider the p-530ARCAT vector, which expresses the CAT reporter gene under control of the (−530, +500) segment of the human AR promoter. The relation between HeLa and LNCaP cells regarding [p300·GABP·N-box_{AR}], where N-box_{AR} refers to the endogenous AR gene, also holds for the p-530ARCAT vector. Following transfection, N-boxes in the p-530ARCAT vector should show increased occupancy in HeLa compared to LNCaP cells (consider points 1 and 3 in FIG. 131).

Consider another vector, p-140ARCAT, which expresses the CAT reporter gene under control of the (−140, +500) segment of the human AR gene. The difference between p-530ARCAT and p-140ARCAT is the (−530, −140) segment, which is exclusively included in the p-53.0ARCAT vector. The (−530, −140) segment shows seven N-boxes at positions (−460, −454), (−381, −375), (−357, −351), (−279, −273), (−243, −237), (−235, −229), and (−224, −218). Assume that at least some of the seven N-boxes bind GABP and suppress AR transcription. Since p-140ARCAT does not include the seven N-boxes, p-140ARCAT should show decreased suppression (consider in the figure points 2 relative to 1 and point 4 relative to 3).

Note that if all cells are transfected with the same concentration of the CAT reporter plasmids (8 μg in following study), the decrease in [p300·GABP·N-box_{AR}] on the two vectors should be the same in all cells. In the figure, the distance measured on the x-axis from point 1 to 2 should be equal to the distance measured from point 3 to 4.

Since [p300·GABP·N-box_{AR}] in HeLa cells is higher than in LNCaP cells, deletion of the AR (−530, −140) segment should increase [Transcription] in HeLa cells more than in LNCaP cells (compare the vertical lines next to the y-axis in the figure). Consider the following observations.

(iii) Observations

(a) Mizokami 1994

A study (Mizokami 1994¹¹³²) measured CAT activity following transfection of p-530ARCAT and p-140ARCAT into HeLa and LNCaP cells. The following table presents the observations (Mizokami 1994, ibid, FIG. 1A).

TABLE 19
Expression level of p-530ARCAT and
p-140ARCAT in HeLa and LNCaP cells.

| | HeLa | | LNCaP | |
|---|---|---|---|---|
| | CAT | Point in | CAT | Point in |
| | expression | figure | expression | figure |
| p-2330ARCAT | 100 | | 100 | |
| p-530ARCAT | 89 ± 25 | point 1 | 49 ± 19 | point 3 |
| p-140ARCAT | 216 ± 159 | point 2 | 60 ± 22 | point 4 |
| Relative increase | 242% | | 122% | |
| in CAT expression | | | | |
| across vectors | | | | |

Note: Mizokami 1994 (ibid) presented the expression levels of p-530ARCAT and p-140ARCAT relative to the expression of the p-2330ARCAT vector, which was set to 100. According to the ΣS model of transcription, p-530ARCAT expression should be higher in LNCaP compared to HeLa cells. However since Mizokami 1994 (ibid) set the expression of p-2330ARCAT to the same value in both HeLa and LNCaP cells, the actual observed expression in the two cell types, and hence the predicted cross-cell type difference, is unavailable for analysis.

As predicted, HeLa cells showed a larger increase in CAT activity following deletion of the seven N-boxes compared to LNCaP cells.

The observations in Mizokami 1994 (ibid) are consistent with the ΣS model of transcription, and with GABP suppression of AR transcription.

(b) S-Shaped Signaling

(i) Single Complex

Consider Agentᵢ. Denote the signal produced by Agentᵢ with Signalᵢ. Denote signal intensity with [Signalᵢ] (brackets denote intensity). The f_{Complex}_{₁}_{-Signal}_{ᵢ} function relates [Complex₁·Box₁] and [Signalᵢ],
[Complex₁·Box₁]=f_{Complex}_{₁}_{-Signal}_{ᵢ}([Signalᵢ]) Function 42

Assume that for every Signalᵢ, the f_{Complex}_{₁}_{-Signal}_{ᵢ} function can be represented by an increasing S— shaped function.

Inserting function f_{Complex}_{₁}_{-Signal}_{ᵢ} into f_{G-Complex}_{₁} yields f_{G-Complex}_{₁}_{-Signal}_{ᵢ}. Symbolically,
f_{G-complex}_{₁}_{-Signal}_{ᵢ}=f_{G-complex}_{₁}°f_{Complex}_{₁}_{-Signal}_{ᵢ} Function 43
or,
[Transcription]_{G}=f_{G-Complex}_{₁}_{-Signal}_{ᵢ}([Signalᵢ]) Function 44

Since f_{Complex}_{₁}_{-Signal}_{ᵢ} is an increasing S-shaped function, f_{G-Complex}_{₁}_{-Signal}_{ᵢ} is also S-shaped. Moreover, since f_{Complex}_{₁}_{-Signal}_{ᵢ} is increasing, the direction of f_{G-Complex}_{₁}_{-Signal}_{ᵢ} is determined by f_{G-Complex}_{₁}. For instance, if f_{G-Complex}_{₁} is increasing, f_{G-Complex}_{₁}_{-Signal}_{ᵢ} is increasing.

Let Complex₁ denote a suppresser. The S-shaped function representing the effect of Complex, on transcription can be divided into three regions. The first region is called “empty boxes” (see Region 1, “[Complex₁·Box₁]=0” in FIG. 132). For every [Signalᵢ] in this region, a decrease in Signalᵢ intensity produces no decrease in suppression, and therefore, no increase in [Transcription]. The second region is the called “full boxes” (see Region 2, “[Complex₁·Box₁]=Max” in FIG. 132). For every [Signalᵢ] in this region, an increase in Signalᵢ intensity produces no increase in suppression and no decrease in [Transcription]. The third region is called “variable boxes” (see Region 3, “0<[Complex₁·Box₁]<Max” in FIG. 132). For every [Signalᵢ] in this region, an increase or decrease in Signalᵢ intensity results in decreased or increased [Transcription], respectively.

Define a “Stripe” as the difference in [Transcription] between [Complex₁·Box₁]=0 and [Complex₁·Box₁]=Max. Let Complex₂ denote a stimulator not regulated by Signalᵢ. An increase in [Complex₂·Box₂] increases the size of the stripe (consider Stripe 1 and Stripe 2 in figure). Note that if Complex₂ is a necessary stimulator, and [Complex₂·Box₂]=0, the S-shaped curve is transformed into an horizontal line (a line is a special case of an S-shaped curve).

(ii) N complexes

(a) Model

Let f_{G-complex}_{_{K}}_{-Signal}_{ᵢ} represent the effect of Signalᵢ on transcription directed through the Complexₖ·Boxₖ complex. Define Aggregate [Transcription] as follows:

$${Aggregate [ Transcription ]}_{G} \\ = {f}_{G - {complex}_{1} - {Signal}_{i}} ( [ {Signal}_{i} ] ) + … + \\ {f}_{G - {complex}_{N} - {Signal}_{i}} ( [ {Signal}_{i} ] ) \\ = {\sum }_{J = 1}^{n} {f}_{G - {complex}_{N} - {Signal}_{i}} ( [ {Signal}_{i} ] ) \qquad Function 45$$

Aggregate [Transcription]G represents the combined effect on transcription of all complexes responsive to Signalᵢ. Note that the function representing Aggregate [Transcription]G is a sum of S-shaped functions, hence, the ΣS name of the model.

Assume N 2. Then,
Aggregate[Transcription]_{G}=f_{G-complex}_{₁}_{-Signal}_{ᵢ}([Signalᵢ])++f_{G-complex}_{₂}_{-Signal}_{ᵢ}([Signalᵢ]) Function 46

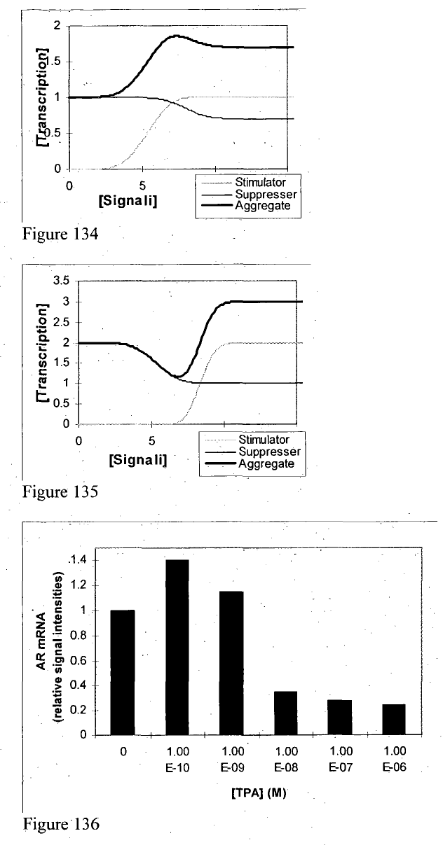

Assume Complex₁ is a suppressor, and Complex₂ is a stimulator of G transcription. According to the ΣS model of transcription, the individual curves representing the relation between [Transcription] and [Signalᵢ] for both Complex₁ and Complex₂, are S-shaped. However, the curve representing the relation between Aggregate [Transcription]_{G} and [Signalᵢ] can take many possible shapes. Call the set of all possible shapes the “topography.” Consider FIG. 133, FIG. 134, and FIG. 135 as examples. The graphs are drawn to scale.

The slope at any given point on the aggregate curve is a sum of the slopes of the individual curves. The aggregate slope is greater than zero at a given point (locally increasing curve), if the size of the stimulator slope is greater than the size of the suppresser slope at that point. The opposite holds for a locally decreasing aggregate slope. The aggregate slope is zero (horizontal line), if the sizes of the stimulator and suppresser slopes are equal.

Common to these shapes is the existence of a range where increasing [Signalᵢ] decreases Aggregate [Transcription]. The existence of such range depends on the existence of a suppresser. Without a suppresser, Aggregate [Transcription] increases monotonically (increases over the entire range).

Note that if a study observes a negative relation between signal intensity and transcription rate, that is, an Aggregate [Transcription] function with a decreasing range, according to the ES model of transcription, the regulators of transcription must include a suppresser. However, since the aggregate curve also includes an increasing section, measuring a positive relation over a given range does not exclude the existence of a suppresser. An observed positive relation is not a counter example for, the existence of a suppresser. It may simply reflect a section where the degree of stimulation is greater than the degree of suppression.

(b) Predictions and Observations: Endogenous Genes

The following studies measure transcription rate of an endogenous gene over a range of signal intensities.

(i) Androgen Receptor (AR) Gene and TPA

Consider the AR gene and the signal produced by treatment with TPA (PMA, phorbol ester).

Observations in some studies suggest that GABP suppresses AR transcription (see chapter on alopecia, p 404). TPA increases ERK phosphorylation in a variety of cells, and, most likely, also in Sertoli cells (Ree 1999¹¹³³ showed TPA induced activation and increased transcription of PKC in Sertoli cells). Since an increase in ERK phosphorylation increases [p300·GABP], an increase in signal intensity produced by TPA treatment of Sertoli cells regulates AR transcription through the AR N-box. However, TPA might regulate AR transcription through other DNA binding sites. Assume a case where TPA suppresses AR transcription through the p300·GABP·N-box_{AR} complex and stimulates transcription through at least one other complex. In other words, assume the signal produced by TPA is shared by the suppressing p300·GABP·N-box_{AR} complex and another stimulating complex. According to the ΣS model of transcription, the curve representing the relation between treatment with TPA and Aggregate [Transcription]_{AR} in Sertoli cells should show a shape included in the topography. Consider the following observations.

A study (Ree 1999, ibid) measured AR mRNA in Sertoli cells following 6 hours treatment with various concentrations of TPA. FIG. 136 presents the results (Ree 1999, ibid, FIG. 5).

Assume no change in AR mRNA stability. Then, the change in mRNA levels indicates a change in transcription. In such a case, the results show a shape similar to the early ridge in the topography (see above, p 394).

The observations in Ree 1999 (ibid) are consistent with the ES model of transcription, and with GABP suppression of AR transcription.

(ii) AR Gene and FSH

Consider the AR gene and the signal produced by treatment with follicle-stimulating hormone (FSH).

FSH stimulates ERK phosphorylation in a dose dependent manner. See, for instance, FSH treatment of oocytes (Su 2001¹¹³⁴, FIGS. 1 and 2) and granulosa cells (Seger 2001¹¹³⁵, Babu 2000¹¹³⁶, FIG. 5B, Das 1996¹¹³⁷, Table 1 and 2, Cameron 1996¹¹³⁸ Table 2). Assume a similar effect of FSH in Sertoli cells. Since an increase in ERK phosphorylation increases [p300·GABP], an increase in signal intensity produced by FSH treatment of Sertoli cells regulates AR transcription through the AR N-box. As with TPA above, FSH might regulate AR transcription through other DNA binding sites. Assume the p300·GABP·N-box_{AR} complex and another stimulating complex share the signal produced by FSH. According to the ΣS model of transcription, the curve representing the relation between treatment with FSH and Aggregate [Transcription]_{AR} in Sertoli cells should show a shape from the topography. Consider the following observations.

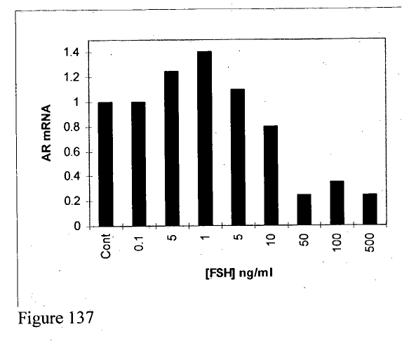

A study (Blok 1992A¹¹³⁹) measured AR mRNA in Sertoli cells from 21-day-old rats following 4 hours of treatment with various concentrations of FSH. FIG. 137 represents the results (Blok 1992A, ibid, FIG. 3)

Assume no change in AR mRNA stability. Then, the change in mRNA levels indicates a change in transcription. In such a case, the results show a shape similar to the early ridge in the topography (see above, p 394).

The observations in Blok 1992A (ibid) are consistent with the ΣS model of transcription, and with GABP suppression of AR transcription.

Notes:

1. In nuclear run-on experiments, the study observed no marked changes in the transcription rate of the AR endogenous gene following 2-4 hours treatment of Sertoli cells with FSH (Blok 1992A, ibid, FIG. 6). As suggested by the authors, the limited sensitivity of run-on assays might be the reason for the observed lack, of change in transcription levels.

2. The study also reports an increase in AR mRNA following 60 minutes of FSH treatment (500 ng/ml) of Sertoli cells from 21 day old rats (Blok 1992A, ibid, FIG. 2). Note that another study (Crepieux 2001¹¹⁴⁰) showed inhibition of ERK phosphorylation in Sertoli cells from 19-day-old rats following incubation with FSH (100 ng/ml) for 15 minutes. After incubation for 60 minutes (60 minutes is the incubation time in Blok 1992A (ibid)), ERK phosphorylation was still lower than controls, although higher than after 15 minutes (Crepieux 2001, ibid, FIG. 7). A decrease in ERK phosphorylation and a corresponding increase in AR transcription is also consistent with GABP suppression of AR transcription.

(iii) 5α-RI Gene and TPA, Ionomycin, IL-6

The (−848, −1) region of the 5 α-reductase type I (5α-RI, SRD5A1) promoter includes ten N-boxes. An overlapping pair at positions (−818, −812), (−814, −808), a pair separated by 25 base pair (bp), or 3 helical turns (HT) at positions (−732, −726) and (−701, −695), a single at position (−661, −655), a pair at positions (−521, −515) (−513, −507), a single at position (−363, −357), and an overlapping pair at positions (−306, −300) (−301, −295). The pair at (−521, −515) (−513, −507) is separated by 2 bp. There are 6 bp the in the N-box and 2 bp distance between the N-boxes, or a total of 8 bp from first nucleotide of the first N-box to first nucleotide of the second N-box. Since there are 10 base pairs per helical turn (HT), or 10 bp per HT, 8 bp is about 1.0 HT. Of the dozens known ETS factors, only GABP, as a tetrameric complex, binds two N-boxes. Typically, the N-boxes are separated by multiples of 0.5 helical turns (see more examples and a discussion in chapters on obesity, p 310, and alopecia, p 404).

Assume 5α-RI is a GABP suppressed gene. Consider the 5α-RI gene and the signal produced by treatment with either TPA, the calcium ionophore ionomycin, or IL-6.

Treatment with either TPA, the calcium ionophore ionomycin, or IL-6 stimulates ERK phosphorylation. (Wilson 1999¹¹⁴¹, FIG. 2C, and Li Y Q 1999¹¹⁴², FIGS. 1 and 2, show increased ERK phosphorylation in Jurkat cells, a human T-cell leukemia cell line, following treatment with TPA. Franklin 2000¹¹⁴³ and Atherfold 1999¹¹⁴⁴ show increased ERK phosphorylation in Jurkat cells following treatment with ionomycin. Daeipour 1993 (ibid) shows increased ERK phosphorylation in AF-10 cells, a human B cell line, following treatment with IL-6.) Since an increase in ERK phosphorylation increases [p300·GABP], an increase in signal intensity produced by treatment with these agents regulates 5α-RI transcription through the N-box. These agents might also regulate 5α-RI transcription through other DNA binding sites. Assume the p300·GABP·N-box_{AR} complex and another stimulating complex share the signal produced by these agents. According to the ES model of transcription, the curve representing the relation between treatment with either TPA, ionomycin, or IL-6, and Aggregate [Transcription]_{5α-RI} in Jurkat cells should show a shape from the topography. Consider the following observations.

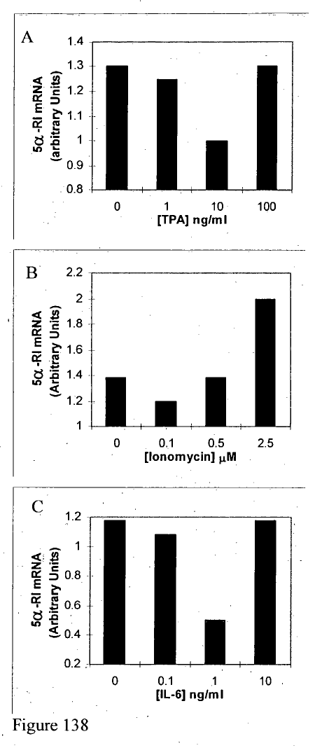

A study (Zhou Z 1999¹¹⁴⁵) measured 5α-RI mRNA levels in Jurkat cells following treatment with various concentrations of TPA, ionomycin, or IL-6. FIG. 138 summarizes the results (Zhou Z 1999, ibid, FIG. 3A,B and FIG. 4B).

Assume no change in 5α-RI mRNA stability. Then, the observed changes in mRNA levels indicate a change in transcription rates. In such a case, the results for all three agents show a shape similar to the early gorge in the topography (see above, p 394).

The observations in Zhou Z 1999 (ibid) are consistent with the ΣS model of transcription, and with GABP suppression of 5α-RI transcription.

The following studies compare transcription before and after a single change in signal intensity.

(iv) AR Gene and Cycloheximide

Gonadal tissues, and specifically rat Sertoli cells, are the only tissues that express high levels of c-mos (Herzog 1989¹¹⁴⁶). A study demonstrated that in mouse oocytes c-mos activates ERK through activation of MEBK and inhibition of a protein phosphatase (Verlhac 2000¹¹⁴⁷, FIG. 9). Treatment of oocytes with the translation inhibitor cycloheximide (CX) decreased expression of c-mos and decreased ERK phosphorylation (Hochegger 2001¹¹⁴⁸, FIG. 6A, see also Moos 1996¹¹⁴⁹). Similar inhibition of c-mos expression and ERK phosphorylation was demonstrated in starfish eggs following treatment with emetine, another translation inhibitor (Sasaki 2001¹¹⁵⁰). Based on the observations in oocytes and starfish eggs, it is reasonable to assume that cycloheximide also inhibits ERK phosphorylation in Sertoli cells. According to the ΣS model of transcription, the curve representing the relation between treatment with cycloheximide and Aggregate [Transcription]_{AR} in Sertoli cells should show a shape from the topography. Consider the following observations.

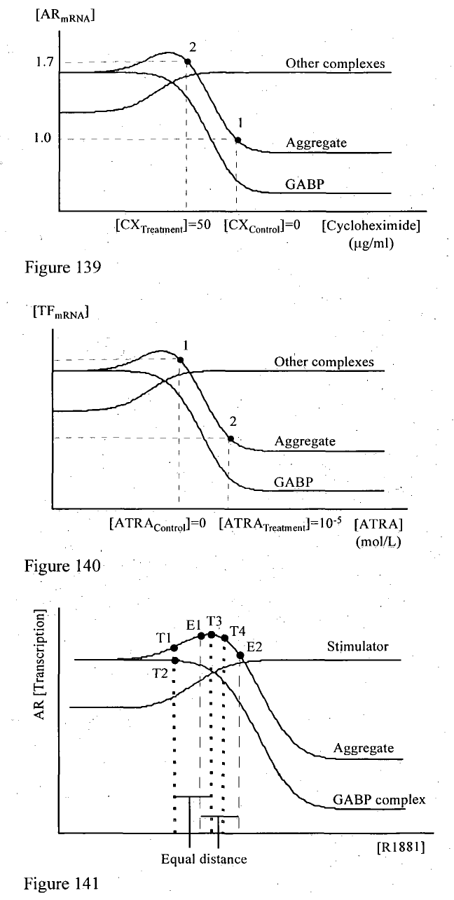

A study (Blok 1992A, ibid) measured AR mRNA in Sertoli cells from 21-day-old rats before and after 4 hours culture in the presence of cycloheximide (50 μg/ml). The results showed an increase in AR mRNA following culture with cycloheximide (Blok 1992A, ibid, FIG. 5). FIG. 139 presents the results in the context of the ΣS model of transcription.

Cycloheximide treatment increased AR mRNA 1.7±0.4-fold. Assume no change in stability of AR mRNA. Then, the observed change in mRNA levels indicates a change in transcription. In such a case, the observations show a curve with a decreasing region indicating inhibition by a suppresser. The observations are consistent with the ES model of transcription, and with GABP suppression of the AR gene.

(v) TF Gene and ATRA

Consider the tissue factor (TF) gene and the signal produced by treatment with all-trans retinoic acid (ATRA).

The effect of ATRA on ERK phosphorylation in THP-1 cells is unknown. However, treatment of in HL-60, another human myeloid leukemia cell line, with retinoic acid increased ERK phosphorylation (Yen 2001¹¹⁵¹, Wang X 2001¹¹⁵², Hong 2001¹¹⁵³, Yen 1999, ibid). Based on the observations in HL-60 cells, it is reasonable to assume that ATRA also increases ERK phosphorylation in THP-1 cells. As an ERK agent, ATRA increases formation of the p300·GABP·N-box_{TF} complex. Observations in some studies suggest that GABP suppresses TF transcription (see chapter on atherosclerosis, p 161). According to the ΣS model of transcription, the curve representing the relation between treatment with ATRA and Aggregate [Transcription]_{TF} should show a shape from the topography. Consider the following observations.

A study (Oeth 1998¹¹⁵⁴) measured TF mRNA levels in THP-1 cells before and after 30 minutes incubation with ATRA (10⁻⁵ mol/L). The results showed a decrease in TF mRNA following treatment with ATRA (Oeth 1998, ibid, FIG. 3A). FIG. 140 presents the results in the context of the ΣS model of transcription.

Assume no change in stability of TF mRNA. Then, the observed change in mRNA levels indicates a change in transcription rate. In such a case, the results show a curve with a decreasing region indicating involvement of a suppresser. The results are consistent with the ES model of transcription, and with GABP suppression of TF transcription.

In principle, an increase in ERK phosphorylation can decrease TF transcription through some mechanism other than the GABP complex. For instance, c-Fos/c-Jun, c-Rel/p65 and Sp1 also regulate TF transcription. However, the study showed no change in binding of these factors to their respective sites following 30 minutes ATRA treatment (10⁻⁵ mol/L) of THP-1 cells (Oeth 1998, ibid, FIG. 6). Another transcription factor that regulates TF transcription is Egr1. ERK stimulates Egr1 activity, and Egr1, in turn, stimulates TF transcription. Therefore, if ATRA stimulates Egr1, the ATRA induced increase in ERK phosphorylation should have increased, and not decreased, TF transcription. Moreover, the study showed that ATRA did not stimulate TF transcription in THP-1 cells (Oeth 1998, ibid, FIG. 2A, first and second column), or in freshly isolated human monocytes (Oeth 1998, ibid, FIG. 1A, first and second column), and, hence, most likely did not activate Egr1. Overall, the observations suggest that ATRA, most likely induced a decrease in TF transcription through an increase in [p300·GABP·N-box_{TF}].

In FIG. 3A (Oeth 1998, ibid), the stimulator of TF transcription, which induced the high baseline [Transcription]_{TF} point 1 in figure above), was unknown. In contrast, the following experiments specifically use LPS as stimulator of TF transcription in THP-1 cells. According to the ΣS model of transcription, the curve representing the relation between treatment with ATRA and [Transcription]_{TF} in the LPS treated cells should show a shape from the topography. Consider the following observations.

To test the effect of ATRA on TF transcription in LPS treated THP-1 cells, Oeth 1998 (ibid) performed nuclear run-on experiments. The study first incubated THP-1 cells with LPS (10 μg/ml) for 1 hour. The results showed an increase in rate of TF transcription (Oeth 1998, ibid, FIG. 5). In a follow-up experiment, the study treated the cells for 30 minutes with ATRA (10⁻⁵ mol/L) before LPS stimulation. The results showed decreased TF transcription relative to the LPS treated cells. Moreover, TF transcription was not only decreased relative to LPS treated cells but also relative to unstimulated cells (Oeth 1998, ibid, FIG. 5). Such a decrease in TF transcription indicates that ATRA is a “general” suppresser of TF transcription and not a specific inhibitor of the LPS signal (a specific LPS signal inhibitor can, at most, eliminate the effect of LPS on TF transcription but not lead to a lower than initial level of transcription). According to the ΣS model of transcription, the individual effect of the stimulating and suppressing complexes can be represented by S-shaped curves in all cells, specifically, LPS treated cells. Hence, the results of this study can be presented graphically in a figure similar to the figure above (the only difference is cell type, untreated THP-1 cells vs. LPS treated THP-1 cells).

The observations in Oeth 1998 (ibid) are consistent with the ES model of transcription, and with GABP suppression of TF transcription.

(c) Predictions and Observations: Transfected Genes

(i) AR Gene and R1881 Androgen

Consider the androgen receptor (AR) gene and the signal produced by treatment with the androgen R1881.

The pSLA3-H2/3-E3k vector expresses LUC under control of the (−1400, +966) segment of the AR promoter. Following transfection into LNCaP cells, microcompetition between the transfected AR promoter and endogenous genes, including AR, decreases availability of GABP to the transfected promoter. Consider the case of empty N-boxes on the transfected promoter, that is, [p300·GABP·N-box_{transfected AR}]=0. Basal Aggregate [Transcription] of LUC following transfection should be represented by a point (point T1 in FIG. 141) corresponding to a point on the suppresser curve positioned in the empty boxes region (point T2 in FIG. 141). FIG. 141 presents the predicted effect of treatment with increasing doses of R1881 on AR [Transcription] according to the ΣS model of transcription.

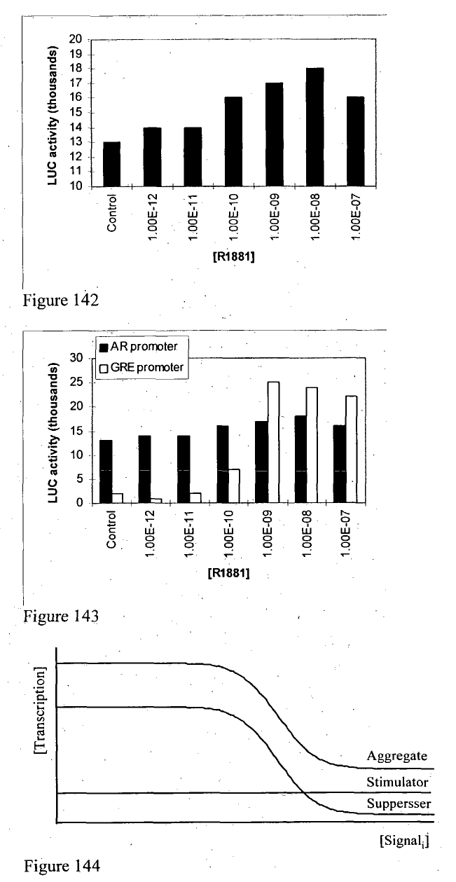

A study (Blok 1992B¹¹⁵⁵) transfected the pSLA3-H2/3-E3k vector into LNCaP cells. Following transfection, the cells were incubated for 24 hours in the presence of R1881. FIG. 142 presents the resulting LUC activity (Blok 1992B, ibid, based on FIG. 6).

As predicted, the curve representing the observations is similar to the curve representing the prediction (compare the results to the T1-T4 region in FIG. 141).

The results are consistent with the ES model of transcription, and with GABP suppression of AR transcription.

Using nuclear run-on experiments, the study also tested the effect of R1881 treatment on transcription of the endogenous gene. LNCaP cells were cultured in the presence of R1881 (10⁻⁸ M) for 8 or 24 hours. The results from the run-on assays showed that transcription of the endogenous AR gene decreased to 85% and 73% of control levels after 8 and 24 hours, respectively (Blok 1992B, ibid, FIG. 7).

In FIG. 142, the effect of 24 hours treatment with 10⁻⁸ M R1881 on transfected and non-transfected cells is illustrated by the shift from point T1 to T3, and from point E1 to E2, respectively. Since R1881 concentration and incubation time are the same in both experiments, the horizontal distance between T1 and T3, and between E1 and E2 should be the same (see figure). According to the figure, 24 hours treatment with 10⁻⁸ M R1881 should increase transcription of the transfected AR gene (consider the shift from point T1 to T3), while the same treatment of the same cells should decrease transcription of the endogenous AR gene (consider the shift from point E1 to E2). The prediction is consistent with the observations reported in Block 1992A.

Note that point E1 is positioned in the increasing region of the aggregate curve, or together with E2, in the region characterized by low negative slopes. Such positions translate, at most, to a moderate decrease in transcription of the endogenous AR gene. Note that Blok 1992A (ibid) described the observed effect of the R1881 treatment as “moderate.”

Blok 1992A (ibid) also reports transfection of LNCaP cells with the pSLA3-GRE-Oct vector, which includes a glucocorticoid response element (GRE) in front of the minimal Oct-6 promoter fused to the LUC reporter gene. Since pSLA3-GRE-Oct, most likely does not include a suppressing N-box, microcompetition between the transfected promoter and endogenous genes should not induce high basal LUC expression. Relative to pSLA3-H2/3-E3k, the AR driven vector, pSLA3-GRE-Oct should show lower basal LUC activity.

As expected, LUC activity in pSLA3-GRE-Oct transfected cells was 15% of the activity in pSLA3-H2/3-E3k transfected cells (see FIG. 143 based on Blok 1992B, ibid, FIG. 6).

(ii) TF Gene and ATRA

Consider a signal that is exclusively a suppresser of transcription. In such a case, the stimulator curve is an horizontal line. Consider FIG. 144.

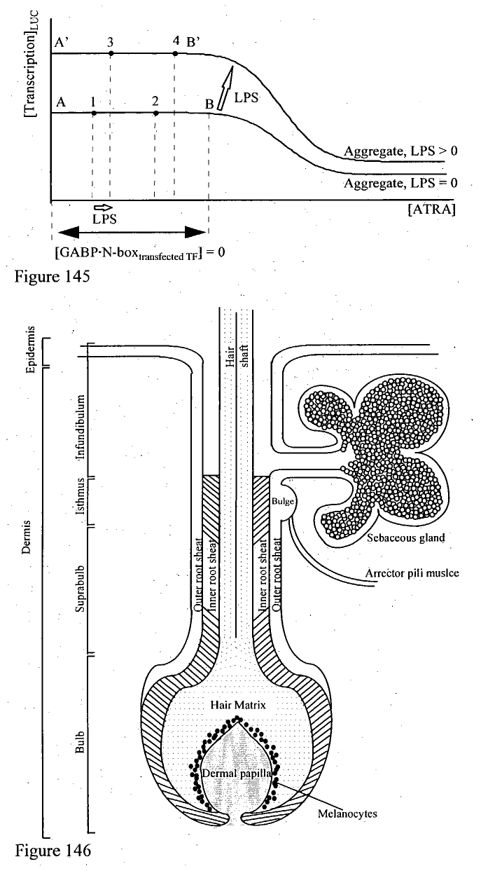

Consider a vector that expresses the reporter gene LUC under control of the TF promoter. Following transfection, microcompetition between the transfected TF promoter and endogenous genes, including the endogenous TF, decreases availability of GABP to the transfected promoter. Assume empty N-boxes in the transfected TF promoter, symbolically, [p300·GABP·N-box_{transfected TF}]=0. With empty N-boxes, basal [Transcription]Luc should be represented by a point positioned in the empty boxes region (point 1 in FIG. 145). ATRA, most likely, does not activate a stimulator of TF transcription (Oeth 1998, ibid, FIG. 2A, first and second column, see discussion above). FIG. 145 presents the effect of treatment with ATRA on [Transcription]_{LUC} according to the ΣS model of transcription.

Under such conditions, ATRA treatment can result in no decrease in reporter gene rate of transcription (illustrated by the shift from point 1 to 2). Consider the following observations

pTF(−2106)LUC contains the wild-type TF promoter (−2106 to +121 relative to the start site of transcription). The promoter includes the two N-boxes at (−363, −343) and (−191, −172) (see chapter on atherosclerosis, p 161). Oeth 1998 (ibid) transfected pTF(−2106)LUC into THP-1 cells. As predicted, 30 minutes ATRA treatment (10⁻⁵ mol/L) of the transfected cells resulted in no decrease in LUC activity relative to untreated cells (points 1 and 2).

Oeth 1998 (ibid) also tested the effect of a combined ATRA and LPS treatment on THP-1 cells. Incubation of THP-1 cells with LPS alone, specifically 5 hours of incubation time, induced no substantial change in ERK phosphorylation (Willis 1996¹¹⁵⁶, FIG. 3, Durando 1998¹¹⁵⁷). Since a small increase in ERK2 phosphorylation with no increase in ERK1 was observed after 15 minutes incubation time, assume that LPS induces, at most, a small increase in ERK phosphorylation. In addition to the effect on ERK phosphorylation, LPS treatment of THP-1 cells activates TF transcription through, for instance, the NF-κB site. Graphically, the two LPS effects, weak phosphorylation of ERK and activation of NF-κB, can be represented by an upward shift of the Aggregate [Transcription]_{LUC} curve, and a small shift to the right on the new curve (compare point 1 and 3 in FIG. 145).

Consider the effect of combined ATRA and LPS treatment on LUC expression in transfected cells. According to the ES model of transcription, transfected THP-1 cells treated with a combination of ATRA and LPS can show in the same level of LUC transcription as transfected cells treated with LPS alone (illustrated by the shift from point 3 to 4).

As expected, 5 hours of treatment with LPS (10 μg/mL) induced a 5-fold increase in luciferase activity (points 1 and 3). As described in the figure, 30-minute treatment with ATRA before the 5-hour treatment with LPS showed no decrease in the LPS induced increase in LUC transcription (points 3 and 4) (Oeth 1998, ibid, FIG. 8).

The observations in Oeth 1998 (ibid) are consistent with the ES model of transcription, and with GABP suppression of TF transcription.

14. Alopecia

a) Microcompetition Susceptible Genes

(1) Androgen Receptor (AR) Gene

(a) AR is a GABP Suppressed Gene

The following observations indicated that AR is a GABP suppressed gene.

(i) N-boxes

The (−381, −1) region of the AR promoter includes seven N-boxes at positions (−381, −375), (−357, −351), (−279, −273), (−243, −237), (−235, −229), (−224, −218), and (−103, −97). Among the seven boxes, a triple and a pair are located within a short distance of each other measured in base pairs (bp) or helical turns (HT). The pair at (−381, −375) and (−357, −351) is separated by 18 bp. There are 6 bp the in the N-box and 18 bp distance between the N-boxes, or a total of 24 bp from first nucleotide of the first N-box to first nucleotide of the second N-box. Since there are 10 base pairs per helical turn (HT), or 10 bp per HT, 24 bp is about 2.5 HT. The three N-boxes at (−243, −237), (−235, −229), and (−224, −218) are separated by 2 and 5 bp, or about 1.0 HT.

Based on the distances, the seven N-boxes are named the pair, the first single, the triple, and the last single. Consider the following table.

TABLE 20
N-boxes in the (−381, −1) region of the AR promoter.

| Name | Position |
|---|---|
| Pair | (−381, −375), (−357, −351) |
| First single | (−279, −273) |
| Triple | (−243, −237), (−235, −229), (−224, −218) |
| Last single | (−103, −97) |

Of the dozens of known ETS factors, only GABP binds, as a tetrameric complex, two N-boxes. Typically, the N-boxes are separated by multiples of 0.5 helical turns (see more examples and a discussion in chapter on obesity, p 310).

(ii) Nested Transfection of Promoter Regions

Two studies isolated a number of DNA regions from the human AR promoter, fused the DNA regions to a reporter gene, transfected the fused vectors into various cells, and measured reporter gene expression. Table 21 summarizes the results.

The observations in Takane 1996 (ibid) and Mizokami 1994 (ibid) show increased AR promoter activity following deletion of promoter segments that include N-boxes. The observations are consistent with GABP suppression of AR transcription.

TABLE 21
Observed effects of AR promoter segments that
include N-boxes on AR promoter activity.

| | | | Smaller promoter region | |
|---|---|---|---|---|
| | | | N-boxes missing in | |
| | | Larger promoter region | smaller promoter | Relative decline |
| | | Large promoter activity | Small promoter activity | in promoter activity |
| Study | Cells | (LPA) | (SPA) | (LPA/SPA) |
| Takane | T47D | (−571, +304) | (−278, +304) | |
| 1996¹¹⁵⁸ | | | pair, first single | |
| | | 116 | 164 | 116/164 = 0.71 |
| | | (−278, +87) | (−146, +87) | |
| | | | triple | |
| | | 6 | 22 | 6/22 = 0.27 |
| | | (−146, +87) | (−74, +87) | |
| | | | last single | |
| | | 22 | 131 | 22/131 = 0.17 |
| Mizokami | HeLa | (−530, +500) | (−140, +500) | |
| 1994 | | | pair, first single, | |
| (ibid) | | | triple | |
| | | 89 | 216 | 89/216 = 0.41 |
| | LNCaP | (−530, +500) | (−140, +500) | |
| | | | pair, first single, | |
| | | | triple | |
| | | 49 | 60 | 49/60 = 0.82 |

(iii) ERK and Endogenous AR Gene Expression

(a) Prediction

Consider a GABP suppressed gene G. An increase in concentration of an ERK agent decreases the concentration of mRNA_{G} (see chapter on signaling and allocation, p 330), assuming the agent does not modify mRNA_{G} stability, and does not modify transcription of G through additional mechanisms, such as modification of other transcription factors. Consider the following sequence of quantitative events.
↑[Agent]→↑[ERK^{phos}]→↑[GABP^{phos}]→↑[p300·GABP]→↓[mRNA_{G}]
Sequence of quantitative events 109: Predicted effect of an ERK agent on transcription of a GABP regulated gene.

(b) Observations

If GABP suppresses AR transcription, an agent that increases ERK phosphorylation should decrease transcription of the AR gene. Consider the observations reported in the following studies. The observations are presented in two tables. The first table lists agents that increase ERK phosphorylation. The second table shows that these agents decrease AR transcription.

TABLE 22
Agents that increase ERK phosphorylation.

| | | | Effect on |
|---|---|---|---|
| Agent | Study | Cells | [ERK^{phos}] |
| Testosterone | Brown JW | SW-13 | ↑[ERK^{phos}] |
| | 2001¹¹⁵⁹ | human adrenal | Table 1, |
| | | carcinoma | |
| DHT | Peterziel | primary genital skin | ↑[ERK^{phos}] |
| | 1999¹¹⁶⁰* | fibroblasts | |
| | | primary prostatic | ↑[ERK^{phos}] |
| | | stormal cells | |
| | | LNCaP | ↑[ERK^{phos}] |
| | | | FIG. ID |
| | | | FIG. 2: dose |
| | | | dependent |
| R1881 | Zhu 1999¹¹⁶¹ | PMC42 | ↑[ERK^{phos}] |
| (androgen) | | human breast cancer | FIG. 1: time |
| | | cells | dependent |
| | | | FIG. 2: dose |
| | | | dependent |
| Flutamide | Zhu 1999 (ibid) | PMC42 | ↑[ERK^{phos}] |
| (antiandrogen) | | human breast cancer | |
| | | cells | |
| EGF | Guo 2000¹¹⁶²** | LNCaP | ↑[ERK^{phos}] |
| | | | FIG. 1B, |
| | | PC-3 | ↑[ERK^{phos}] |
| | Kue 2000¹¹⁶³ | PC-3 | ↑[ERK^{phos}] |
| | Chen T 1999¹¹⁶⁴ | LNCaP | ↑[ERK^{phos}] |
| | | | FIG. 1A, B |
| | Putz 1999¹¹⁶⁵ | LNCaP | ↑[ERK^{phos}] |
| | | DU145 | ↑[ERK^{Phos}] |
| TPA | Chen T 1999 | LNCaP | ↑[ERK^{phos}] |
| | (ibid) | | |
| TNFα | | A variety of cell | ↑[ERK^{phos}] |
| | | types | |
| Serum (20%) | Chen T 1999 | LNCaP | ↑[ERK^{phos}] |
| | (ibid) | | |
| | Guo 2000 (ibid) | LNCaP | Low basal |
| | | | ERK^{phos} in |
| | | | serum |
| | | | free medium |
| | Guo 2000 (ibid) | PC-3 | Low basal |
| | | | ERK^{phos} in |
| | | | serum |
| | | | free medium |
| | Kue 2000 (ibid) | PC-3 | ↑[ERK^{phos}] |
| | Magi-Galluzzi | high-grade prostatic | ↑[ERK^{phos}] |
| | 1998¹¹⁶⁶ | intraepithelial | |
| | | neoplasia | |
| | | (precursor of prostate | |
| | | cancer) | |
| | Kue 2000 (ibid) | PC-3 | ↑[ERK^{phos}] |
| *The study also showed an increase in Elk-1 transcription activity following treatment with the androgen DHT, or the antiandrogens casodex and hydroxyflutamide. ERK phosphorylation activates Elk-1, which is a member of the ETS family. Therefore, the increase in Elk-1 | | | |
| # activity also indicates a possible increase in ERK phosphorylation by the treatments. The increase in Elk-1 transcription activity was dependent on the presence of AR. In contrast, Elk-1 activation by EGF, another ERK agent, was independent of AR. | | | |

**The study reports no increase in ERK phosphorylation in LNCaP or PC-3 cells following treatment with DHT.

The study also showed an increase in Elk-1 transcription activity following treatment with the androgen DHT, or the antiandrogens casodex and hydroxyflutamide. ERK phosphorylation activates Elk-1, which is a member of the ETS family. Therefore, the increase in Elk-1 activity also indicates a possible increase in ERK phosphorylation by the treatments. The increase in Elk-1 transcription activity was dependent on the presence of AR. In contrast, Elk-1 activation by EGF, another ERK agent, was independent of AR.
The study reports no increase in ERK phosphorylation in LNCaP or PC-3 cells following treatment with DHT.

Table 23 shows that treatment with the above listed ERK agents decreased AR mRNA. Since the agents can also decrease mRNA through a decrease in mRNA stability, the table lists the studies that specifically measured transcription using run-on experiments. As predicted, treatment with agents that stimulate ERK phosphorylation decreased transcription of the AR gene.

Notes:

1. The studies referenced in the table measure the effect of the listed agents on transcription of the endogenous AR gene. For studies that measure the effect of ERK agents on a transfected AR gene, see chapter on ΣS, p 390.

TABLE 23
Effect of ERK agents on AR mRNA levels.

| ERK agent | Study | Cells | Effect on [mRNA_{AR}] |
|---|---|---|---|
| DHT | Mizokami | LNCaP | ↓[mRNA_{AR}] |
| | 1992¹¹⁶ | | (FIG. 2A) |
| | Yeap | LNCaP | ↓[mRNA_{AR}] |
| | 1999¹¹⁶⁸ | | (FIG. 3B) |
| | | | Decreased transcription |
| | | | in run-on assays |
| | | | Increased mRNA half life |
| | Yeap 1999 | MDA453 | ↓[mRNA_{AR}] |
| | (ibid) | | (FIG. 3C) |
| | | | No change in run-on |
| | | | assays |
| | | | Decreased mRNA half life |
| Testosterone | Quarmby | LNCaP | ↓[mRNA_{AR}] |
| | 1990¹¹⁶⁹ | | |
| R1881 | Quarmby | LNCaP | ↓[mRNA_{AR}] |
| (methyltrienolon [symbol] | 1990 (ibid) | | |
| synthetic | | | |
| androgen) | | | |
| Cyproterone | Quarmby | LNCaP | ↓[mRNA_{AR}] |
| acetate | 1990 (ibid) | | |
| (antiandrogen) | | | |
| EGF | Henttu | LNCaP | ↓[mRNA_{AR}] |
| | 1993¹¹⁷⁰ | | FIG. 3, FIG. 7 |
| | | | FIG. 4: time dependent |
| TPA | Ree 1999 | Sertoli | ↓[mRNA_{AR}] |
| | (ibid) | 19 days | FIG. 4: time dependent |
| | | old rats | FIG. 5: dose dependent |
| TNFα | Mizokami | LNCaP | ↓[mRNA_{AR}] |
| | 2000¹¹⁷ | | Dose dependent |
| | | | No change in run-on |
| | | | assays |
| | Sokoloff | LNCaP | ↓[mRNA_{AR}] |
| | 1996¹¹⁷² | | |
| | Henttu | LNCaP | ↓[mRNA_{AR}] |
| | 1993 (ibid) | | |
| Serum (10% | Quarmby | LNCaP | ↓[mRNA_{AR}] |
| fetal | 1990 (ibid) | | |
| calf serum) | | | |

2. If AR is a GABP suppressed gene, cells with a constitutive increase in ERK phosphorylation should show low expression of AR. Consistent with such prediction, two studies (Segawa 2001¹¹⁷³, Putz 1999, ibid) reported no AR expression in DU145 cells, which show constitutively active ERK2.

3. Some papers reported increased AR mRNA following treatment with an ERK agent. For instance, one study (Kumar 1998¹¹⁷⁴) showed an increase in mouse AR mRNA following treatment with TPA. The study identified a TPA response element in the AR promoter that drives the increase in AR mRNA. Such element could not be found in the human AR gene. Another study (Chen T 1999, ibid) showed that IL-6 treatment of LNCaP cells increased ERK phosphorylation. However, in contrast to other ERK agents, treatment with IL-6 decreased AR mRNA in LNCaP cells (Lin 2001¹¹⁷⁵, FIG. 7). (In addition to the increase in AR mRNA, FIG. 7 presents another surprising result. In contrast to other studies, the figure shows no AR mRNA in untreated, control LNCaP cells.) A possible explanation for the unexpected observation might be the IL-6 phosphorylation of Stat3. Stat3 binds AR (Chen T 2000¹¹⁷⁶) and might induce an increase in AR transcription offsetting the decrease in AR mRNA induced by the increase in GABP suppression.

(iv) AR Mediated Cellular Events

(a) Effect on Cell Proliferation and Differentiation

(i) Prediction

Dermal papilla cells express AR (Diani 1994¹¹⁷⁷ Ando 1999¹¹⁷⁸). Let AG denote androgen, CN, cell number, subscript DP, “in a dermal papilla cell,”: (for instance, CN_{DP} denotes dermal papilla cell number), CD, cell differentiation, ₚAR, androgen receptor protein, and _{mRNA}AR, androgen receptor mRNA. Consider the following sequence of quantitative events.

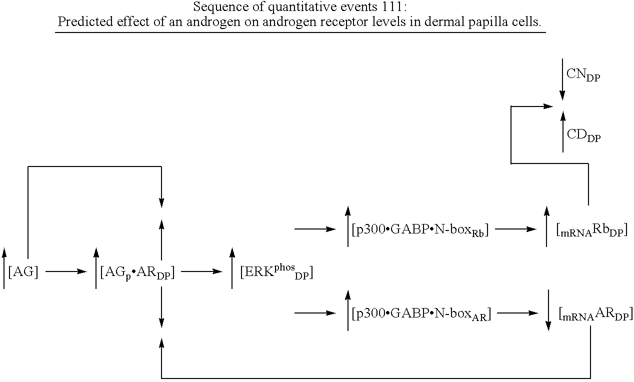

[ₚAR_{DP}] denotes the concentration of androgen receptor protein in dermal papilla cells. Androgen can either increase [ₚAR_{DP}], since androgen stabilizes AR protein, decrease [ₚAR_{DP}], since androgen decreases AR mRNA, or maintain the level of [ₚAR_{DP}], if the effects cancel each other out. Consider a case where ↑[Androgen]→↑[AG·ₚAR_{DP}], that is, an increase in androgen concentration that increases the concentration of androgen bound to the androgen receptor. In such a case, the increase in androgen concentration should decrease dermal papilla cell number. Treatment of dermal papilla cells with androgen should decrease cell proliferation. Consider the following observations.

(ii) Observations

A study (Kiesewetter 1993¹¹⁷⁹) measured growth rates of papilla cells grown in control medium, or medium supplemented with testosterone (345 nM), or DHT (345 nM) for 14 days. The results showed an increase in doubling time, decrease in cell number per dish, and decrease in ³H-thymidine incorporation for both treatments. As expected, both androgens significantly decreased papilla cell proliferation. The study also showed decreased outer root sheath (ORS) keratinocyte proliferation relative to interfollicular keratinocytes, and relative to cells cultured in control medium.

Note: Another study (Obana 1997¹¹⁸⁰) reports no effect of testosterone on dermal papilla cell proliferation when cultured alone (10⁻¹⁰ to 10⁻⁷ M testosterone concentrations, data not shown), or co-cultured with outer root sheath cells (10⁻¹⁰ M testosterone concentration, table 2). The seemingly conflicting results are actually consistent with the observations of Kiesewetter 1993 (ibid). According to Kiesewetter 1993 (ibid) testosterone concentrations “lower than 173 nM” (1.73×10⁻⁷ M) produced no significant effect on papilla cell proliferation (Kiesewetter 1993, ibid, FIG. 2). Only concentrations higher than 1.73×10⁻⁷ M, specifically 3.45×10⁻⁷ M (Kiesewetter 1993, ibid, Table I), decreased proliferation.

Sebocytes also express AR (Diani 1994, ibid, Choudhry 1992¹¹⁸¹). Hence, the prediction should also hold for sebocytes.

A study (Deplewski 1999¹¹⁸²) isolated sebocytes from preputial glands of young adult male Sprague-Dawley rats, and measured their cell proliferation following treatment with DHT (10⁻⁶ M). The results showed a 40% decrease in DNA synthesis measured by ³H-thymidine uptake relative to untreated controls (Deplewski 1999, ibid, FIG. 3B). By measuring lipid accumulation in sebocyte colonies, the study also evaluated the effect of DHT on cell differentiation. The results showed a small increase (statistically insignificant) in sebocyte differentiation following DHT treatment (Deplewski 1999, ibid, FIG. 2B). The DHT effect on sebocyte differentiation was amplified to statistically significant levels in the presence of insulin (10⁻⁶M) (Deplewski 1999, ibid, FIG. 2A).

Both dermal papilla cells and sebocytes express AR. As expected, androgen treatment of both AR expressing cell types decreased proliferation and increased differentiation.

(2) 50α Reductase, Type I (5α-RI) Gene

(a) 5α-RI is a GABP Suppressed Gene

Some evidence shows that 5α-RI is a GABP suppressed gene (see chapter on ΣS, p 390).

Note: FSH receptor knockout (FORKO) mice showed higher expression of 3β-hydroxysteroid dehydrogenase (3β-HSD) (Krishnamurthy 2001¹¹⁸³) FSH is an ERK agent. Gene activation, in an ERK agent deficient environment, is consistent with suppression by GABP (other animal models for ERK agent deficient environments include, for instance, the OB mouse with the mutation in the ERK agent leptin, the Zucker rat with the mutation in the receptor for the ERK agent leptin, see also chapter on obesity, p 310).

(3) Human sIL-1ra Gene

(a) Human sIL-1ra is a GABP Stimulated Gene

Human secretory interleukin-1 receptor antagonist (sIL-1ra) is a GABP stimulated gene (Smith 1998, ibid).

b) Male Pattern Alopecia (MPA)

MPA is also called male pattern baldness (MPB), and androgenic alopecia (AGA).

(1) Introduction

(a) Hair Follicle

(i) Anatomy

FIG. 146 describes the structure of the hair follicle, also called pilosebaceous unit.

(ii) Life Cycle

A hair follicle perpetually cycles through three stages: growth (anagen), regression (catagen), and rest (telogen). In anagen, formation of the new lower hair follicle begins with proliferation of secondary germ cells in the bulge. During middle anagen, (anagen VI), matrix cells, which produce the hair shaft, proliferate at a rate comparable to bone marrow and intestinal epithelium. At the end of anagen, the matrix keratinocytes cease proliferation, and the hair follicle enters catagen. During catagen, the hair follicle goes through a process of involution. Toward the end of catagen, the dermal papilla condenses and moves upward coming to rest underneath the bulge. During telogen, the hair shaft matures into a club hair, composed of non-proliferating, terminally differentiated keratinocytes. The club hair is shed from the follicle during the next growth cycle.

In human scalp, anagen lasts approximately 3-4 years, catagen, 2-3 weeks, and telogen 3 months. Approximately 84% of scalp hair follicles are in anagen, 1-2%, in catagen, and 10-15% in telogen.

(iii) Dihydrotestosterone (DHT) Synthesis

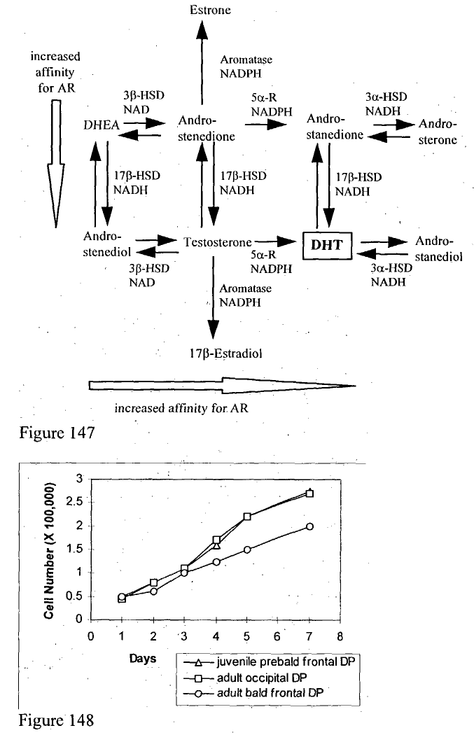

DHEA is a 19-carbon steroid hormone secreted primarily by the adrenal glands. DHEA is synthesized from pregnenolone, a cholesterol derivative. DHEA is converted to dehydroepiandrosterone sulfate (DHEAS), the predominant form circulating in plasma. In the hair follicle, DHEA is metabolized to DHT (see FIG. 147).

Abbreviations:

DHEA—Dehydroepiandrosterone
17β-HSD—17β-hydroxysteroid dehydrogenase
3β-HSD—3β-hydroxysteroid dehydrogenase-Δ⁵-Δ⁴-isomerase
3β-HSD—3β-hydroxysteroid dehydrogenase
5α-R—5α Reductase
AR—Androgen receptor

5 α-R occurs in two isoforms, type I (5α-RI), located primarily in sebocytes, and type II (5 α-RII), located primarily in the inner layer of the outer root sheath, and in the inner root sheath of the hair follicle (Thiboutot 2000¹¹⁸⁴, Bayne 1999¹¹⁸⁵, Chen 1998¹¹⁸⁶, Chen W 1996¹¹⁸⁷). 5 α-R metabolizes testosterone into DHT. In hair follicles, the sebaceous glands account for the majority of androgen metabolism (Deplewski 2000¹¹⁸⁸, Table 1). Moreover, sebocytes are the key regulators of androgen homeostasis in human skin (Fritsch 2001¹¹⁸⁹).

(2) Microcompetition with Foreign DNA

Sebocytes are permissive to a latent infection with a GABP virus. A study (Clements 1989¹¹⁹⁰) inoculated male and female Bozzi mice, via the right rear footpads, with 2×10⁶ pfu of HSV-1, a GABP virus. All mice survived and none showed ill effects except for a slight FP swelling for the first few days. Six months after inoculation, a latent viral infection was detected in cells of the sebaceous glands, hair root sheath, and within the epidermis. Another study (Moriyama 1992¹¹⁹¹) showed persistence of HSV-1 in cells of the sebaceous glands. A third study (Okimoto 1999¹¹⁹²) subcutaneously inoculated NIH Swiss mice with 10⁶ pfu Moloney murine leukemia virus (M-MuLV). Four to six weeks post inoculation, an immunohistochemistry analysis detected the M-MuLV capsid antigen in cells of the sebaceous glands and of the outer root sheath (ORS).

Consider sebocytes infected with a GABP virus. The viral DNA increases the number of N-boxes in infected cells. Microcompetition with viral N-boxes disrupts transcription of cellular genes. The following sections present predicted effects of the disrupted transcription on a molecular, cellular, and clinical level, and compare the predicted effects with observation reported in studies with MPA patients.

(3) Mechanism Based Predictions and Observations

The following sections use symbolic presentations. In these presentations, subscript “S” denotes “synthesized in, or expressed by a sebocyte.” For instance, AR_{S} means “androgen receptor expressed by a sebocyte,” and DHT_{S} means “DHT synthesized by a sebocyte.” Subscript “DP” denotes the same for a dermal papilla cell. For instance, AR_{DP} means “androgen receptor expressed by a dermal papilla cell.”

(a) Sebaceous Gland Hyperplasia

(i) Prediction

Assume sebocytes harbor a latent infection with a GABP virus. Consider the following sequence of quantitative events.

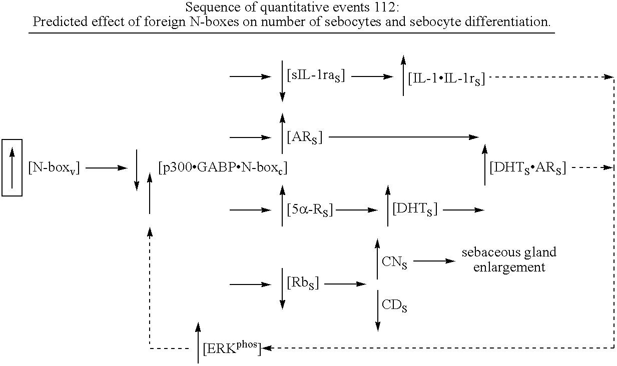

Assume the secondary effects of DHT and IL-1, marked with doted lines, decrease, but do not eliminate the primary effect of microcompetition on [p300·GABP·N-box_{c}]. The different size arrows next to [p300·GABP·N-box_{c}] illustrate this assumption. Under the assumption, microcompetition with a GABP virus increases proliferation and decreases differentiation of infected sebocytes, symbolically, ↑CN_{S} and ↓CD_{S}. Since an increase in sebocyte proliferation results in gland enlargement, microcompetition with a GABP virus results in a larger sebaceous glands.

If MPA results from microcompetition with a GABP virus in infected sebocytes, hair follicles in the balding area of MPA patients should show an increase in number of sebocytes, i.e. sebaceous gland hyperplasia, and larger sebaceous gland. Consider the following observations.

(ii) Observations

A study (Lattanand 1975¹¹⁹³) collected 347 tissue specimens from the balding area of 23 MPA patients. A histopathological analysis showed moderate to marked sebaceous gland enlargement in 76% of the specimens (Lattanand 1975, ibid, FIG. 2, 4, 5). The gland showed no atrophy. According to Lattanand and Johnson (1975, ibid): “a prominent enlargement of sebaceous glands was a constant feature in our material of the middle and late stages of MPA.”

Another study (Puerto 1990¹¹⁹⁴) reports that “histological controls of our biopsies demonstrated that in alopecic area sebaceous glands occupy the greater part of the tissue, accounting for 80%, whereas in hairy skin these glands were of normal size, accounting for about 15% of the pieces.” In a follow-up study, the authors describe the observation as “hyperplastic glands” (Giralt 1996¹¹⁹⁵).

As expected, hair follicles in the balding area of MPA patients showed sebaceous gland hyperplasia, sebaceous gland enlargement, and no cell atrophy.

(b) Sebaceous Gland Centered T-cell Infiltration

(i) Background: IL-1

The IL-1 family includes the IL-1α and IL-1β cytokines, the type I and II receptors, denoted IL-1RI and IL-1RII, respectively, and the IL-1 receptor antagonist, denoted IL-1ra. Two major structural variants of IL-1ra have been described: a secreted isoform, sIL-1ra, and an intracellular isoform, icIL-1ra. A single gene, under control of different promoters, transcribes both isoforms. According to a recent review on IL-1ra (Arend 1998¹¹⁹⁶) “sIL-1ra protein is produced by virtually any cell that is capable of synthesizing IL-1, possible with the exception of endothelial cells and hepatocytes.” Sebocytes express IL-1α and IL-1β (Anttila 1992¹¹⁹⁷). Hence, it is reasonable to assume that sebocytes synthesize sIL-1ra. Moreover, consistent with the assumption, a study showed constitutive expression of sIL-1ra in all rabbit tissue examined, including lung, liver, spleen, thymus, caecum, kidney, heart, brain, and specifically skin (Matsukawa 1997¹¹⁹⁸, FIG. 2). In addition, another study, although not specific to the secreted isoform, showed expression of IL-1 ra in sebaceous glands (Kristensen 1992¹¹⁹⁹).

IL-1 is not a potent chemoattractant. However, IL-1 induces expression of the potent chemoattractant growth regulated oncogene-α (GROα, melanoma growth-stimulatory activity (MGSA), cytokine-induced neutrophil chemoattractant (CINC), neutrophil-activating protein-3 (NAP-3), KC, N51) by stimulating its transcription through activation of the NF-κB transcription factor, and by stabilizing the chemoattractant mRNA (Tebo 2000¹²⁰⁰, Awane 1999¹²⁰¹, Hybertson 1996¹²⁰², Koh 1995¹²⁰³). GROα is a chemoattractant for both T-cells and neutrophils (Fujimori 2001¹²⁰⁴, Jinquan 1995¹²⁰⁵, Aust 2001¹²⁰⁶). Sebocytes express GROα (Tettelbach 1993). Hence, it is reasonable to conclude that an increase in IL-1 concentration around sebocytes chemoattracts T-cells to that region.

(ii) Prediction

Consider the following sequence of quantitative events. [symbol]
[N-boxᵥ]→↓[p300·GABP·N-box_{c}]→↓[sIL-1ra_{Sebo}]→↑[IL-1ₜₒₜₐₗ]/[sIL-1ra_{Sebo}]→↑[T-cell] around the sebaceous gland
Sequence of quantitative events 113: Predicted effect of foreign N-boxes on number of T-cells around the sebaceous gland.

Assume that the GABP virus does not affect IL-1 secretion from infected sebocytes, denoted [IL-1_{Sebo}] Also, assume that other cells in the hair follicle are not infected, and therefore, secrete IL-1 at levels comparable to controls. Denote secretion by other cells with [IL-1_{Other}]. Total IL-1 concentration around infected sebocytes, denoted [IL-1ₜₒₜₐₗ], is equal to [IL-1ₜₒₜₐₗ]=[IL-1_{Sebo}]+[IL-1_{Other}]. Since [IL-1_{Sebo}] and [IL-1_{Other}] are fixed, [IL-1ₜₒₜₐₗ] is fixed. sIL-1ra is a GABP stimulated gene. Therefore, microcompetition with the GABP virus decreases IL-1ra secretion from infected sebocytes. Since [IL-1ₜₒₜₐₗ] is fixed and [sIL-1ra_{Sebo}] decreases, [IL-1ₜₒₜₐₗ]/[sIL-1ra_{Sebo}] increases around infected sebocytes. The decrease in secreted IL-1ra is equivalent to an increase in IL-1 around infected sebocytes. If MPA results from microcompetition with a GABP virus in infected sebocytes, hair follicles from the balding area of MPA patients should show an increase in T-cell concentration around the sebaceous gland. Moreover, the other regions of the hair follicle should show no increase in T-cell concentrations. Consider the following observations.

(iii) Observations

A study (Sueki 1999¹²⁰⁸) collected 6-mm punch biopsy specimens from 19 male MPA patients and 6 normal male controls. The specimens were taken from the area between hairy and balding regions on the vertex, termed the transitional zone between alopecic and non-alopecic scalp. The study also collected hairy specimens from the occipital region of each MPA patient. Histopathological analysis of the transitional specimens showed “patchy inflammatory infiltrates consisting predominantly of lymphoid cells around the lower portion of the infundibulum, isthmus and/or sebaceous glands in all specimens” (Sueki 1999, ibid, FIG. 1A-D). No inflammatory infiltrates were observed around the majority of the bulbs in these specimens. A morphometric analysis showed a significant increase in the number of infiltrates per 0.1 mm² in the transitional zone specimens collected from the MPA patients, relative to controls, and relative to the occipital specimens (Sueki, 1999, ibid, FIG. 2).

Another study (Jaworsky 1992¹²⁰⁹) reports that “in biopsies of transitional scalp, the thin zone of partial hair loss separating non-alopecic and alopecic scalp, lower portions of follicular infundibula showed extensive infiltration by mononuclear cells, the majority of which (>95%) were leu 1-positive T-cells (Jaworsky 1992, ibid, FIG. 2). The infiltrates were centered around infundibular epithelium in the vicinity of the sebaceous duct orifice and near the origins of sebaceous lobules. The lowermost bulbar region of the follicle was uninvolved.”

A third study (Lattanand 1975, ibid) reports: “about one-half of the specimens in this study showed a significant increase of inflammatory cells in MPA.”

As expected, hair follicles in the balding area of MPA patients showed an increase in the number of T-cells around the sebaceous gland, and no change in T-cell concentration around other regions of the hair follicle.

(c) Short Anagen (Premature Catagen)

(i) Background: IL-1 as Catagen Inducer

Several clinical and experimental studies reported observations consistent with IL-1 as inducer of catagen. A study (Hoffmann 1998¹²¹⁰) measured mRNA levels of IL-1α, IL-1β, IL-1RI, IL-RII, and IL-1ra during hair follicle cycling induced by depilation. The results showed an increase in IL-1α and IL-1β mRNA with onset of spontaneous catagen (around day 19), with peak expression during telogen (day 25). Changes in IL-1RI expression paralleled the changes in IL-1α and IL-1, mRNA. Based on these observations, Hoffmann, et al., (1998, ibid) concluded: “our findings are consistent with the concept that IL-1α, IL-1β, and IL-1RI are involved in the control of catagen development.” Another study (Philpott 1996¹²¹¹) tested the effect of treatment with low concentration of IL-1α or IL-1β (0.01-0.1 ng/ml) on the hair follicle. In normal hair follicles, melanocytes are located within the follicle bulb closely surrounding, but not penetrating the dermal papilla. In contrast, IL-1 treated hair follicles showed melanin granules within the dermal papilla (Philpott 1996, ibid). Consistent with Hoffmann's conclusion, Tobin 1998¹²¹² reported that catagen hair follicles exhibited pigment incontinence in the dermal papilla.

Different compartments of the hair follicle express receptors for the IL-1 cytokine, and, therefore, are potential targets for its biological activity. A study (Ahmed 1996¹²¹³) investigated the immunoreactivity of hair follicles to members of the IL-1 family. The study showed intense cellular staining of IL-1RI and variable staining of IL-1ra in the inner root sheath at the border close to the outer root sheath, corresponding to the Henle layer, beginning at the suprapapillary level, and extending into the isthmus and infundibulum (Ahmed 1996, ibid, FIG. 2b and FIG. 1g). The outer root sheath showed weak to moderate staining for the receptors (Ahmed 1996, ibid, FIG. 2a-c), and weak staining for IL-1ra (Ahmed 1996, ibid, FIG. 1g). An earlier study (Deyerle 1992¹²¹⁴), using in situ hybridization, showed expression of IL-1RI, but not IL-1RII in follicular epithelial cells. (Note that dermal papilla cells showed no IL-1RI expression). To summarize, the target cells for IL-1 biological activity in the hair follicle are located in the isthmus and infundibulum regions, in the inner root sheath at the border close to the outer root sheath, and in the other root sheath.

In support of Hoffmann's conclusion about IL-1 as catagen inducer, two other catagen inducers, neurotrophin 3 (NT-3) and transforming growth factor β1 (TGFβ1) (Botchkarev 2000¹²¹⁵, Botchkarev 1998¹²¹⁶, Foitzik 2000¹²¹⁷) target cells in the hair follicle in locations similar to IL-1 (see details below). Moreover, similar to IL-1, NT-3, and TGFβ1 are ERK agents.

Neurotrophin 3 (NT-3) and transforming growth factor β1 (TGFβ1), two other ERK agents, share target cells with IL-1. NT-3 is a member of the neurotrophin family. Two types of receptors mediated the biological effects of NT-3: the tyrosine kinase receptor TrkC, and p75NTR, the low affinity neurotrophin receptor. NT-3 also binds with low affinity to TrkA, the high affinity receptor for the nerve growth factor (NGF), and TrkB, the high-affinity receptor for the brain-derived neurotrophic factor (BDNF/NT-4).

To correlate NT-3 and TrkC expression in situ during hair follicle cycling, a study (Botchkarev 1998, ibid) used immunohistochemistry to assess NT-3 and TrkC immunoreactivity. The study found expression of NT-3 and TrkC in normal mouse skin in hair cycle dependent manner with expression peaking shortly before or during catagen development. Specifically, the study observed NR-3 immunoreactivity in all unmanipulated telogen hair follicles in the innermost outer root sheath, located in close proximity to the hair shaft (Botchkarev 1998, ibid, FIG. 2A). Moreover, during late anagen (anagen IV), NT-3 immunoreactivity became visible in single cells in the isthmus region. In even later anagen (anagen VI), NT-3 immunoreactivity was also observed in the innermost layer of the outer root sheath, in the region of the isthmus where the inner root sheath disappears (Botchkarev 1998, ibid, FIG. 2E). The expression pattern of NT-3 in the upper outer root sheath remained constant during anagen to catagen transformation (Botchkarev 1998, ibid, FIG. 3 summarizes these observations).

Another study (Foitzik 2000, ibid) correlated TGFβ1 and TGFβ receptor II (TGFβRII) expression during hair follicle cycling. The study observed strong expression of TGFβ1 and TGFβRII during late anagen and onset of catagen in the proximal and central regions of the outer root sheath (Foitzik 2000, ibid, FIG. 1 and FIG. 2) (see also Welker 1997¹²¹³).

(ii) Prediction

Assume that development of premature catagen results in shorter anagen or a decrease in anagen time interval. Consider the following sequence of quantitative events. [symbol]
[N-boxᵥ]→↓[p300·GABP·N-box_{c}]→↓[sIL-1ra_{S}]→↑[IL-1ₜₒₜₐₗ]/[sIL-1ra_{Sebo}]→↑Premature catagen development→↓[Anagen time interval]
Sequence of quantitative events 114: Predicted effect of viral N-boxes on length of anagen time interval.

Microcompetition with a GABP virus in infected sebocytes increases [IL-1ₜₒₜₐₗ]/[sIL-1ra_{Sebo}]. If MPA results from microcompetition with a GABP virus in infected sebocytes, hair follicles in the balding area of MPA patients should show shorter anagen. Consider the following observations.

(iii) Observations

A study (Courtois 1994¹²¹⁹) of the human hair cycle gathered data over a period of 14 years in a group of 10 subjects, with or without MPA. The study used the phototrichogram technique to measure the anagen and telogen time interval of each follicle in a group of 100 follicles identified in a 1 cm² scalp area. The technique is not suitable for measuring the brief catagen phase, however, it permits quantification of the latency interval (also called lag) between hair shedding and the onset of anagen. For each subject, the study took two photographs of the same area once a month at a 2-day interval for 144 successive months. The study recorded and characterized about 9,000 hair cycles for a total of about 930 hair follicles followed monthly over more than a decade.

The results showed premature transformation from anagen to telogen resulting in a decreased anagen time interval for a certain proportion of hairs. The proportion increased in size with increased extent of alopecia. The premature transformation from anagen to telogen was associated with an increase in the rate of hair loss. The results also showed parallel decline in hair diameter, and longer latency, leading to a decreased number of hairs on the scalp. The shorter finer (vellus) hair showed even longer and more frequent latency. See also Courtois 1995¹²²⁰.

As expected, MPA is associated with shorter anagen.

(d) Small Dermal Papilla

(i) Prediction

Microcompetition with a GABP virus increases expression of 5α-R in infected sebocytes. As a result, DHT synthesis increases. The extra DHT binds androgen receptors in dermal papilla cells, increasing ERK phosphorylation and Rb transcription. The excess unphosphorylated Rb protein decreases dermal papilla cell proliferation and dermal papilla size. Consider the following sequence of quantitative events. [symbol]
[N-boxᵥ]→↓[p300·GABP·N-box_{5α-R}]→↑[_{mRNA}5α-R₅]→↑[DHT_{S}]→↑[DHT_{S}·AR_{DP}]→↑[p300·GABP·N-box_{Rb}]→↑[Rb_{DP}]→↓[CN_{DP}]→↓DP size
Sequence of quantitative events 115: Predicted effect of viral N-boxes on size of dermal papilla.

If MPA results from microcompetition with a GABP virus in infected sebocytes, hair follicles in the balding area of MPA patients should show decreased dermal papilla cell proliferation and a small dermal papilla. Moreover, since the decreased proliferation depends on excess DHT synthesis in sebocytes, prepubertal dermal papilla cells should show a proliferation rate similar to controls. Consider the following observations.

(ii) Observations

Alopecia in frontal scalps of postpubertal stumptailed macaques is a recognized animal model for human MPA. A study (Obana 1997, ibid) isolated dermal papilla cells from anagen hair follicles of prepubertal juvenile prebald frontal scalp (“juvenile prebald frontal DP”), adult bald frontal scalp (“adult bald frontal DP”), and adult occipital scalp (“adult occipital DP”) of stumptailed macaques. The study then cultured the cells following inoculation at a density of 4×10⁴ cells/35-mm dish. FIG. 148 presents the growth curves of the cultured cells (Obana 1997, ibid, FIG. 2).

After 5 and 7 days in culture, cell number in the “adult bald frontal DP” culture was significantly lower than cell number in “juvenile prebald frontal DP” and “adult occipital DP” cultures. Moreover, during the log phase, the mean population doubling time (69.02±5.92 h) of dermal papilla cells in the “adult bald frontal DP” culture was significantly longer (p<0.01) than those in “juvenile prebald frontal DP” (37.0±1.63 h) and “adult occipital DP” (39.49±4.13 h) cultures (Obana 1997, ibid).

As expected, hair follicles in the balding area showed decreased dermal papilla cell proliferation. Moreover, as expected, prepubertal dermal papilla cells in prebald frontal scalp showed proliferation similar to controls.

The study also recorded the mean length of the dermal papilla measured from dome to base in frontal and occipital hair in juvenile and adult stumptailed macaques. The following table presents the results.

TABLE 24
Length of the dermal papilla measured from dome to base in frontal
and occipital hair in juvenile and adult stumptailed macaques.

| | Frontal scalp | Occipital scalp |
|---|---|---|
| Adult | 75.0 ± 5.2 μm | 153.1 ± 4.8 μm |
| (postpubertal) | n = 12 | n = 12 |
| Juvenile | 81.2 ± 3.7 μm | 84.0 ± 4.4 μm |
| (prepubertal) | n = 12 | n = 12 |

(See also Obana 1997, ibid, FIG. 1.) As expected, alopecia was associated with smaller dermal papilla. Moreover, as expected, the size of the juvenile dermal papilla in frontal scalp was similar to controls.

Another study (Randall 1996¹²²¹) compared proliferation and size of dermal papilla collected from balding and non-balding sites using by-products of normal surgical procedures. The balding samples were obtained from frontal and vertex regions of individuals undergoing corrective surgery for MPA. Non-balding specimens were obtained from the nape of the neck of these patients (similar to occipital hair). The dermal papilla cells were seeded into 35-mm Petri dishes for cell growth studies. To establish primary cultures, microdissected dermal papilla were individually transferred to a 35 mm tissue culture plate supplemented with 20% fetal calf serum (FCS) or 20% human serum (HS). The cells grown out from the dermal papilla to subculture were seeded into 35-mm Petri dishes treated with FCS or HS, and counted every 2-3 days over a 14-day period.

FIG. 149 presents the results (Randall 1996, ibid, FIG. 4b). As expected, the results showed slower growth of balding dermal papilla cells compared to non-balding cells under both growth conditions. The study also measured the size of isolated dermal papilla. As expected, the results showed a 50-75% decrease in size of balding compared to non-balding dermal papilla (Randall 1996, ibid, FIG. 2).

Another study (Alcaraz 1993¹²²²) measured dermal papilla cell number in normal and balding scalp of MPA patients. The results showed a significant decrease in the number of dermal papilla cell nuclei per unit volume in scalps with established baldness compared to controls. The total number of papilla cell nuclei in follicles from alopecic scalp was about 50% of normal scalp (Alcaraz 1993, ibid, FIG. 2). The study also measured dermal papilla volume. The results showed an inverse relation between volume and degree of alopecia.

See also a recent review discussing the relation between dermal papilla size and MPA (Whiting 2001¹²²³).

As expected, hair follicles in the balding area of MPA patients showed decreased dermal papilla cell number and dermal papilla volume.

(e) Extended Lag

(i) Background: DHT as Delayer of Anagen Onset

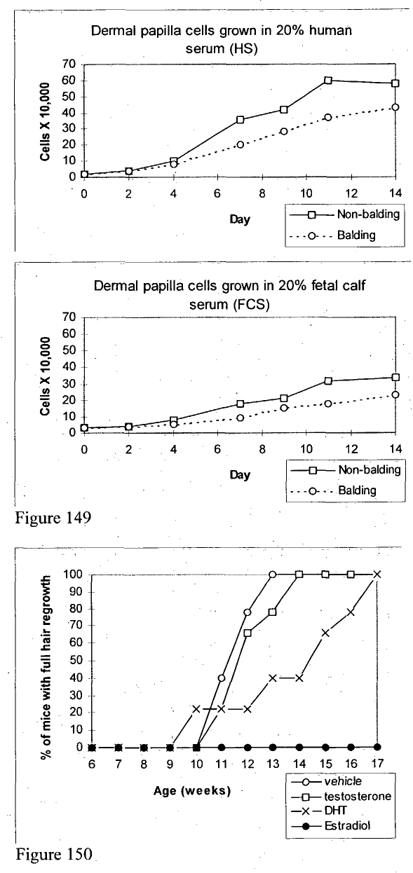

The hair follicle cycle of mice is highly synchronized from birth to 12 weeks of age showing fixed periods of anagen, telogen, and catagen. The second telogen in CD-1 mice begins at about 6 weeks of age and lasts until about 9 weeks of age, at which time synchronous onset of the third anagen can be observed. To measure the effect of certain agents on onset of anagen, a study (Chanda 2000¹²²⁴) clipped hair in the dorsal region (about 4×2.5 cm area) of female CD-1 mice with electric clippers. At 6 weeks of age, when hair follicles are synchronously in their second telogen, the study started applying, topically, 10 nmol of testosterone, DHT, 17β-estradiol, or acetone vehicle alone. The treatment was repeated twice weekly until week 17. The effect of treatment on hair regrowth is summarized in FIG. 150 (Chanda 2000, ibid, FIG. 1A).

Vehicle treated control mice regrow a full coat of hair by week 13. Mice treated with testosterone showed a small delay in hair regrowth, whereas mice treated with DHT showed a 3-4 week delay. Mice treated with 17β-estradiol showed an indefinite delay in hair regrowth. These observations indicate that DHT delays the onset of anagen. Since the latency interval, or lag, is defined as the time between hair shedding and onset of anagen, higher DHT concentrations extend the lag.

(ii) Prediction

Consider the following sequence of quantitative events. [symbol]
[N-boxᵥ]→↓[p300·GABP·N-box_{5α-R}]→↑[_{mRNA}5α-R_{S}]→↑[DHT_{S}]→↑Delay in onset of anagent→↑Lag
Sequence of quantitative events 116: Predicted effect of viral N-boxes on the lag between hair shedding and onset of anagen.

Microcompetition with a GABP virus increases expression of 5α-R in infected sebocytes. As a result, infected sebocytes increase DHT synthesis. The increase in DHT increases the delay in onset of anagen, which increases the lag. If MPA results from microcompetition with a GABP virus in infected sebocytes, hair follicles in the balding area of MPA patients should show an extended lag. Consider the following observations.

(iii) Observations

Courtois 1994 (ibid) reported that hair follicles in the balding area of MPA male patients showed an extended lag (see description of study above). See also Courtois 1995 (ibid).

Guarrera 1996¹²²⁵ called a hair follicle during the lag phase “empty space.” In monthly phototrichograms of two women with Ludwig type I-II patterned baldness for 2 years the study observed higher number and longer lasting “empty spaces” in the women with more severe alopecia. Based on this observation, Guarrera and Rebora (1996, ibid) concluded: “in Ludwig I and II patterned baldness, the increase in lag duration may be important in the balding process.”

As expected, hair follicles in the balding area of MPA patients show extended lag, or long lasting empty spaces.

Research indicates that the DP produces a signal that initiates anagen and directs the bulge follicular stem cells to divide (Oh 1996, ibid). Dermal papilla cells express AR in telogen (Diani 1994, ibid, Choudhry 1992, ibid). Assume that the intensity of the signal produced by dermal papilla cells is a function of the number of these cells. Then, the decreased proliferation of the dermal papilla cells (see above) decreases signal intensity and delays the onset of anagen. The decreased proliferation is a result of excess ERK phosphorylation in DP cells. Consistent with this model, treatment with 17β-estradiol, another ERK agent with receptors in DP cells during telogen, also delayed the onset of anagen (see figure above and observations in Oh 1996¹²²⁶, Smart 1999¹²²⁷) The stronger effect of estradiol might be explained by a stronger effect, relative to DHT, on ERK phosphorylation in DP cells during telogen. See also discussion regarding the relation between dermal papilla size and lag duration in Whiting 2001 (ibid).

(f) Increased AR Expression in Sebocytes

(i) Prediction

Consider the following sequences of quantitative events. [symbol]
[N-boxᵥ]→↓[p300·GABP·N-box_{AR}]→↑[_{mRNA}AR_{S}]
Sequence of quantitative events 117: Predicted effect of viral N-boxes on adrogen receptor mRNA levels in sebocytes.

Microcompetition with a GABP virus increases AR expression in infected sebocytes. If MPA results from microcompetition with a GABP virus in infected sebocytes, hair follicles in the balding area of MPA patients should show increased AR expression in sebocytes. Consider the following observations.

(ii) Observations

A study (Sawaya 1989¹²²⁸) collected specimens of bald scalp from men with MPA undergoing hair transplant or scalp decrease surgery (“bald-surgery”). Specimens of balding scalp were also collected from male trauma victims at autopsy within 3 hours post-mortem (“bald-autopsy”). At autopsy, specimens of hairy scalp were also collected and used as controls (“non-bald”).

Sebaceous glands were isolated by manual dissections under a microscope. Binding of the [³H]DHT and [³H]methyltrienolone (R1881) androgens in the sebocyte cytosol fraction was measured using dextran coated charcoal and sucrose gradient methods. Table 25 summarizes the observed dissociation constant (Kd), and binding capacity (Bmax) (Sawaya 1989, ibid, Table I).

TABLE 25
Observed dissociation constant (Kd) and binding capacity (Bmax)
of the [³H]DHT and [³H]methyltrienolone (R1881) androgens
in sebocytes isolated from balding and non-balding scalps.

| | | | [3H]methyltrienolone | |
|---|---|---|---|---|
| | [3H]DHT | | (R1881) | |
| | | Bmax | | Bmax |
| | | fmol/mg | | fmol/mg |
| | Kd nM | protein | Kd nM | protein |
| Bald-surgery | 0.79 ± 0.04 | 34.1 ± 14.1 | 0.90 ± 0.08 | 30.1 ± 4.3 |
| Bald-autopsy | 0.95 ± 0.09 | 27.0 ± 3.1 | 0.90 ± 0.03 | 26.8 ± 3.0 |
| Non-bald | 1.89 ± 0.79 | 20.0 ± 4.6 | 2.05 ± 0.56 | 18.7 ± 4.4 |

The balding specimens showed lower Kd and higher Bmax compared to non-balding specimens indicating stronger affinity and greater binding capacity, respectively, for the tested androgens in the cytosol of sebocytes from the balding relative to the non-balding specimens. The observations are consistent with increased AR expression in balding specimens.

The study also measured androgen content in nuclei of the isolated sebocytes. The following table summarizes the results (Sawaya 1989, ibid, Table IV).

TABLE 26
Observed dissociation constant (Kd) and binding
capacity (Bmax) of AR Type I and II in sebocytes
isolated from balding and non-balding scalps.

| | AR Type I | | AR Type II | |
|---|---|---|---|---|
| | | Bmax fmol/mg | | Bmax fmol/mg |
| | Kd nM | protein | Kd nM | protein |
| Bald | 0.68 | 311 | 8.0 | 1,786 |
| Non-bald | 0.55 | 239 | 8.5 | 665 |

The balding and non-balding specimens showed similar dissociation constants. However, the balding specimens showed higher Bmax relative to the non-bald specimens, consistent with increased androgen content in balding specimens.

As expected, the study reports observations consistent with increased sebocyte expression of androgen in hair follicles in the balding area of MPA patients.

(g) Decreased AR Expression in Dermal Papilla Cells

(i) Prediction

Consider the following sequence of quantitative events. [symbol]
[N-boxᵥ]→↓[p300·GABP·N-box_{5α-R}]→↑[_{mRNA}5α-R_{S}]→↑[DHT_{S}]→↑[DHT_{S}·AR_{DP}]→↑[ERK^{phos}_{DP}]→↑[p300·GABP·N-box_{AR}]→↓[_{mRNA}AR_{DP}]
Sequence of quantitative events 118: Predicted effect of viral N-boxes on androgen receptor mRNA levels in dermal papilla cells.

Microcompetition with a GABP virus in infected sebocytes decreases AR expression in dermal papilla cells. If MPA results from microcompetition with a GABP virus in infected sebocytes, hair follicles in the balding area of MPA patients should show decreased AR expression in dermal papilla cells. Consider the following observations.

(ii) Observations

A study (Hodgins 1991¹²²⁹) measured AR protein concentration in dermal papilla cells isolated from vertex and occipital scalp skin obtained from healthy balding and non-balding men. AR concentrations were 13.67±2.55, 17.5±6.75, and 20.89±13.18, for the balding, occipital, and non-balding specimens, respectively (mean±SD) (p=0.063 for the difference between “balding” and “non-balding” specimens, and p=0.032 for the difference between “balding” and “non-balding”+“occipital” specimens) (Hodgins 1991, ibid, data taken from FIG. 1). As expected, dermal papilla cells showed significantly lower AR protein concentrations in balding compared to non-balding vertex regions.

Another study (Hibberts 1998¹²³⁰) measured a significantly higher level of androgen receptors (Bmax) in primary lines of cultured dermal papilla cells derived from balding compared to non-balding scalp (Hibberts 1998, ibid, FIG. 3). As stated, these results are inconsistent with the predicted decrease in dermal papilla cell AR expression, and with the results reported in Hodgins 1991 (ibid). However, a comparison of the data in Hibberts 1998 (ibid) and Hodgins 1991 (ibid) may suggest another conclusion.

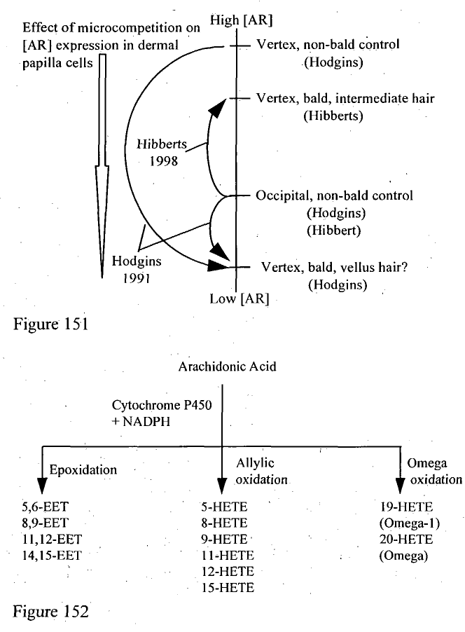

Hodgins 1991 (ibid) compared balding vertex dermal papilla cells to non-balding vertex papilla cells. Unlike Hodgins 1991 (ibid), Hibberts 1998 (ibid) compared balding vertex cells to non-balding occipital cells, which show lower AR concentration relative to vertex non-balding cells. [AR] in dermal papilla cells isolated from occipital and vertex non-balding cells were 17.5±6.75, n=6, and 20.89±13.18, n=9, respectively (Hodgins 1991, ibid). Moreover, two studies (Ando 1999, ibid, and Itami 1995¹²³¹) showed very low levels of AR in dermal papilla cells isolated from occipital scalp hair. In addition, Hibberts 1998 (ibid) used dermal papilla isolated from intermediate and not vellus follicles (Hodgins 1991 (ibid) provides no description of the hair follicles). Consider FIG. 151.

Although Hibberts 1998 (ibid) measured a higher AR concentration in vertex balding follicles relative to occipital follicles, if the study would have compared the vertex balding concentrations to vertex non-balding concentrations, the results would have probably been similar to those reported in Hodgins 1991 (ibid).

The use of occipital hair as non-balding controls is standard in MPA research. In cross tissue analysis, use of such controls might provide insightful information. However, in dynamic analysis, where a study wishes to compare biological entities “before and after” a disruption modifies their environment, use of occipital hair as control, or as the “before,” might be misleading.

Moreover, according to the prediction, the increase in DHT synthesis in sebocytes increases ERK phosphorylation in DP cells, which decreases AR mRNA. However, since DHT also stabilized AR protein, a study can still observe elevated AR protein in DP cells in MPA.

(4) Transitive Deduction

(a) DHT

(i) Microcompetition Decreases DP Size

Microcompetition with a GABP virus in infected sebocytes decreases dermal papilla cell proliferation and dermal papilla size (see above). Symbolically, [symbol]
[N-boxᵥ]→↓[CN_{DP}]→↓DP size
Sequence of quantitative events 119: Predicted effect of viral N-boxes on size of dermal papilla.

(ii) Decrease in DP Size Increases Hair Loss

A study showed a correlation between size of the dermal papilla and hair diameter (Elliott 1999¹²³²). Moreover, according to a recent review (Whiting 2001, ibid), “In androgenic alopecia, follicles undergo miniaturization, shrinking from terminal to vellus-like hairs. . . . When does follicular miniaturization occur in androgenic alopecia? It may occur at some stage in early catagen or early anagen. . . . Follicular miniaturization does not occur during established anagen, since anagen hairs maintain the same diameter during each hair cycle, nor in the telogen where there is no metabolic activity. . . . How does miniaturization occur? It is unlikely that rapid hair loss in androgenic alopecia can be explained simply by a series of progressively shorter anagen cycles. . . . An important factor here is the size of the dermal papilla, which determines the size of both hair bulb matrix and hair shaft. Human follicle dermal papilla miniaturization is the direct result of decrease in papillary cell numbers.” However, since “cell loss by apoptosis has not been reported in dermal papilla cells in normal cycling,” it is likely that the decreased size is a result of decreased cell proliferation (see above). To conclude, “it is hypothesized that the miniaturization seen with pattern hair loss may be the direct result of decrease in the cell number and, hence, size of the dermal papilla.”

Assume a decrease in dermal papilla size increases hair loss. Symbolically,
↓DP size→↑[hair loss]
Sequence of quantitative events 120: Predicted effect of dermal papilla size on hair loss.

(iii) Logical Summary

According to the principle of transitive deduction,
If (↑[N-boxᵥ]→↓DP size) AND (↓DP size→↑[Hair loss]) Then (↑[N-boxᵥ]→↑[hair loss])

Since microcompetition decreases dermal papilla size, and since a decrease in dermal papilla size increases hair loss, microcompetition with a GABP virus in infected sebocytes increases hair loss.

(iv) Dermal Papilla, ERK Agents and Hair Loss

(a) Prediction

Microcompetition with a GABP virus in infected sebocytes increases DHT expression, which increases ERK phosphorylation in DP cells. Consider an agent With a similar effect on ERK phosphorylation in DP cells.

Let “dermal papilla ERK agent” (DP ERK agent) denote an agent that increases ERK phosphorylation in dermal papilla cells. Note that treatment of a pilosebaceous unit with such agent also increases ERK phosphorylation in sebocytes, which decreases expression of 5α-RI, decreases DHT synthesis, and decreased ERK phosphorylation in DP. Assume the direct effect on ERK phosphorylation in DP cells is larger then the effect mediated thought DHT, that is, assume a greater than zero “net” effect of the DP ERK agent on [ERK^{phos}_{DP}]. Call such agent, “net” DP ERK agent. Consider the following sequence of quantitative events. [symbol]
[Agent]→↑[ERK^{phos}_{DP}]→↑[p300·GABP·N-box_{Rb}]→↑[Rb_{DP}]→↓[CN_{DP}]→↑[Hair loss]
Sequence of quantitative events 121: Predicted effect of a net DP ERK agent on hair loss.

According to the principle of transitive deduction, microcompetition with a GABP virus in infected sebocytes increases sebocyte synthesis of DHT, which increases hair loss. Similar to DHT, treatment with another net DP ERK agent should also increase hair loss. Consider the following observations.

(b) Observations

(i) Treatment of Isolated Hair Follicles

TPA, the calcium ionophore A 23187, TNFα, testosterone, and estrogen increase ERK phosphorylation in a variety of cells. Assume that these agents also increase ERK phosphorylation in dermal papilla cells. As DP ERK agents, they should decrease hair growth in isolated hair follicles.

A study (Harmon 1995¹²³³) isolated anagen hair follicles from scalp skin of females undergoing facelift surgery, and placed the isolated hair follicles in suspension culture. Treatment with TPA resulted in potent, dose-dependent inhibition of total cumulative hair follicle growth (IC₅₀=1 nM) (Harmon 1995, ibid, FIG. 1). Another study (Hoffmann 1997¹²³⁴) isolated scalp hair from 20 healthy volunteers. Intact, viable anagen hair was isolated by microdissection and placed in culture for 6 days. Presence of the calcium ionophore A 23187 (2 μM), or TPA (1 μM) significantly inhibited hair growth (Hoffmann 1997, ibid, FIG. 1). A third study (Philpott 1996, ibid) reported inhibition of scalp hair growth following treatment of isolated hair follicles with TNFα (Philpott 1996, ibid, FIG. 1). Finally, a study (Kondo 1990¹²³⁵) observed similar growth inhibition of isolated hair follicles following treatment with testosterone or estrogen.

As expected, treatment of isolated hair follicles with a variety of DP ERK agents decreased hair growth.

(ii) Topical Application

The studies described above used isolated hair follicles. In contrast, the following studies reported the effect of topical, in vivo, application of a DP ERK agent on hair growth. According to Chanda 2000 (ibid), topical application of the DP ERK agent 17β-etradiol decreased hair growth (see study details above, see also Oh 1996, ibid). As expected, topical application of a DP ERK agent decreased hair growth.

(b) IL-1

(i) Viral N-boxes and [IL-1]/[IL-1ra]

Microcompetition with a GABP virus in infected sebocytes decreases [sIL-1 ra_{Sebo}], which increases [IL-1]/[IL-1ra] in the hair follicle (see above). Symbolically, [symbol]
[N-boxᵥ]→↑[IL-1]/[IL-1ra]
Sequence of quantitative events 122: Predicted effect of viral N-boxes on the ratio between interleukin 1 and interleukin 1 receptor antagonist.

(ii) [IL-1]/[1L-1ra] and Hair Loss

Several studies reported observations consistent with IL-1 as inducer of hair loss (see, for instance, a recent review, Hoffmann 1999¹²³⁶).

A study (Groves 1995¹²³⁷) generated two lines of transgenic mice (TgIL-1.1 and TgIL-1.2), which overexpress IL-1α in basal keratinocytes. TgIL-1.2 mice, which had lower levels of transgene expression and milder phenotype compared to TgIL-1.1, showed pronounced sparseness of hair, particularly over the scalp and the base of the tail. Unlike TgIL-1.1 mice, TgIL-1.2 mice showed no spontaneous focal cutaneous inflammatory lesions. Moreover, although TgIL-1.2 mice showed a diffuse increase in dermal mononuclear cells, hair follicles were relatively unaffected. These observations indicate that a mild increase in IL-1α expression might result in loss of seemingly normal scalp hair.

Another study (Hoffmann 1997, ibid) isolated scalp hair from 20 healthy volunteers. Intact, viable anagen hair was isolated by microdissection and placed in culture. Six days of incubation with IL-1 (100 ng per ml) significantly inhibited hair growth (Hoffmann 1997, ibid, FIG. 1). Philpott 1996 (ibid) also reported inhibition of scalp hair growth following treatment of isolated hair follicles with IL-1α or IL-β (Philpott 1996, ibid, FIG. 1). Xiong 1997¹²³⁸ also reported similar IL-1β induced growth inhibition of isolated scalp hair.

The observations in these studies suggest that an increase in [IL-1]/[IL-1ra] increases hair loss. Symbolically,
↑[IL-1]/[IL-1ra]→↑[Hair loss]
Sequence of quantitative events 1239: Predicted effect of the ratio between interleukin 1 and interleukin 1 receptor antagonist on hair loss.

(iii) Logical Summary

According to the principle of transitive deduction:
If (↑[N-boxᵥ]→↑[IL-1]/[IL-1ra]) AND (↑[IL-1]/[IL-1ra]→↑[hair loss]) Then (↑[N-boxᵥ]→↑[Hair loss])

Since microcompetition increases [IL-1]/[IL-1ra], and since an increase in [IL-1]/[IL-1ra] increases hair loss, microcompetition with a GABP virus in infected sebocytes increases hair loss.

c) MPA and Other Chronic Diseases

(1) MPA and Cardiovascular Disease

(a) Prediction

Infection with a GABP virus increases susceptibility to atherosclerosis (see chapter on atherosclerosis, p 161). Atherosclerosis increases susceptibility to cardiovascular disease. If MPA results from microcompetition with a GABP virus in infected sebocytes, MPA should be associated with cardiovascular disease. Consider the following observations.

(b) Observations

Several recent studies reported an association between MPA and cardiovascular disease initially reported in Cotton 1972²³⁹. Consider the following examples.

A study (Lesko 1993¹²⁴⁰) compared the extent of baldness in men under the age of 55 years admitted to a hospital for a first nonfatal myocardial infarction (n=665) and in controls, men admitted to the same hospitals with noncardiac diagnoses (n=772). The results showed an age adjusted relative risk (RR) of 0.9 (95% confidence interval (95% CI), 0.6-1.3) for myocardial infarction in men with frontal baldness compared to men with no hair loss. However, relative risk (RR) of myocardial infarction in men with vertex baldness was 1.4 (95% CI, 1.2-1.9). Moreover, the results showed an increase in RR of myocardial infarction with the degree of vertex baldness (p<0.01), reaching 3.4 (95% CI, 1.7-7.0) for severe vertex baldness. Based on these observations, Lesko, et al., (1993, ibid) concluded: “these data support the hypothesis that male pattern baldness involving the vertex scalp is associated with coronary artery disease in men under the age of 55 years.”

Another study (Herrera 1995¹²⁴¹) used a Cox proportional hazards regression to evaluate the relation between the extent and progression of baldness, determined in 1956 and in 1962 in a cohort of 2,017 men from Framingham, Mass., and the incidence of coronary heart disease (CHD), CHD mortality, cardiovascular mortality, noncardiovascular mortality, and all-cause mortality in the same cohort during the subsequent 24 years (1962-1986). The results showed lack of association between extent of baldness and occurrence of a cardiovascular event or death. However, for men with rapid progression of baldness, the relative risk, adjusted for age and other cardiovascular disease risk factors, was 2.4 (95% CI, 1.3-4.4) for a coronary heart disease event, 3.8 (95% CI, 1.9-7.7), for coronary heart disease mortality, and 2.4 (95% CI, 1.5-3.8), for all-cause mortality. Based on these observations, Herrera, et al., (1995, ibid) concluded: “rapid hair loss may be a marker for coronary heart disease.”

Another study (Lotufo 2000¹²⁴²) examined the relation, between male pattern baldness and CHD events. A CHD event was defined as nonfatal myocardial infarction (MI), angina pectoris, and/or coronary revascularization. The study asked 19,112 US male physicians aged 40 to 84 years enrolled in the Physicians' Health Study to complete a questionnaire at the 11-year follow-up concerning their pattern of hair loss at age 45 years. All participants were free of CHD at baseline. During the 11 follow-up years, 1,446 CHD events were recorded in this cohort. The results showed an age-adjusted relative risk of CHD equal to 1.09 (95% CI, 0.94-1.25) for men with frontal baldness relative to men with no hair loss. However, RR for men with mild, moderate, or severe vertex baldness was 1.23 (95% CI, 1.05-1.43), 1.32 (95% CI, 1.10-1.59), and 1.36 (95% CI, 1.11-1.67), respectively (p for trend, <0.001). RR of CHD for men with vertex baldness increased with hypertension (multivariate RR=1.79; 95% CI, 1.31-2.44), or high cholesterol levels (multivariate RR=2.78; 95% CI, 1.09-7.12). Multivariate adjustment for age, parental history of MI, height, BMI, smoking, history of hypertension, diabetes, high cholesterol level, physical activity, and alcohol intake, did not significantly change the results. Independent analysis of nonfatal MI, angina, and coronary revascularization, or analysis of events among men older and younger than 55 years at baseline, produced similar results. Based on these observations, Lotufo, et al., (2000, ibid) concluded: “vertex pattern baldness appears to be a marker for increased risk of CHD events, especially among men with hypertension or high cholesterol levels.”

Another study (Matilainen 2001¹²⁴³) measured onset of MPA in all 85 males living on 31 Dec. 1999 in a Finnish town with total population of 7,200, who had had a coronary revascularization procedure between March 1987 and January 1999. The onset of MPA was also measured in individually selected age-matched controls living in the same town. MPA was defined as grade 3 vertex or more on the alopecia classification scale of Hamilton, modified by Norwood. The results showed an unadjusted odds ratio (OR) of 3.57 (95% CI, 1.19-10.72) for coronary revascularization under the age of 60 years in men with early onset of MPA compared to men with late onset of MPA or no hair loss. Unadjusted OR for men at any age was 2.14 (95% CI, 1.08-4.23). OR, adjusted to the traditional cardiovascular disease risk factors, was 3.18 (95% CI, 1.01-10.03). Based on these observations, Matilainen, et al., (2001, ibid) concluded: “our results support the hypothesis that the early onset of androgenic alopecia is a risk factor for an early onset of severe coronary heart disease.”

As expected, MPA is associated with cardiovascular disease.

(2) MPA and Obesity, Insulin Resistance/Hyperinsulinemia

(a) Prediction

Infection with a GABP virus increases susceptibility to obesity, insulin resistance, and hyperinsulinemia (see chapters on obesity, p 310, and signal resistance, p 340). If MPA results from microcompetition with a GABP virus in infected sebocytes, MPA should be associated with obesity and insulin resistance/hyperinsulinemia. Consider the following observations.

(b) Observations

A study (Matilainen 2000¹²¹¹) compared body mass index (BMI) in patients with early-onset MPA (younger than 35 years) and age-matched controls. The 154 cases were men aged 19-50 from a town in Finland with a total population of 7,300, including 1,253 eligible men of that age group. For each case, the study selected an individually age-matched control living in the same town. The results showed strong association between early-onset of MPA and moderate overweight (BMI>27 kg/m², p<0.001, odd ratio (OR) 2.9 CI, 1.76-4.79) or severe overweight (BMI>30 kg/m², p=0.012, OR=2.56, CI, 1.24-4.88). The results also showed a strong association between early-onset of MPA, and antihypertensive (p=0.024), or lipid-lowering (p=0.003) medications. In addition, the results showed a two-fold increase in the risk for hyperinsulinemia (OR=1.91, CI, 1.02-3.56) in men with MPA compared to controls. Based on these observations, Matilainen, et al., (2000, ibid) concluded: “our practice-based case-control study in men aged 19-50 years showed a strikingly increased risk of hyperinsulinemia and insulin-resistance-associated disorders such as obesity, hypertension, and dyslipidemia in men with early onset of alopecia (<35), compared with age-matched controls.”

Another study (Piacquadio 1994¹²⁴⁵) compared BMI in 48 females with MPA, ages 24-48, with BMI in the general population. No MPA patient had a significant medical history or was on medication known to interfere with hair growth. All patients were premenopausal. None had a history of known hormonal abnormalities, including amenorrhea, hirsutism, and polycystic ovarian disease, however, four patients had oligomenorrhea and/or hypomenorrhea of unknown origin. Four patients had undergone hysterectomy without oophorectomy. The results showed a significant increase in BMI compared to the general population. The most striking difference was observed within the morbidly obese category (8.3% of patients vs. 1% in general population). Based on these observations, Piacquadio, et al., (1994, ibid) concluded: “overall, there appeared to be a possible positive correlation between the degree of obesity and the prevalence of alopecia.”

As expected, MPA is associated with obesity, insulin resistance, and hyperinsulinemia.

(3) MPA and Cancer

(a) Prediction

Infection with a GABP virus increases susceptibility to cancer (see chapter on cancer, p 358). If MPA results from microcompetition with a GABP virus in infected sebocytes, MPA should be associated with cancer. Consider the following observations.

(b) Observations

Although some earlier studies failed to show an association between MPA and prostate cancer (see discussions in the two studies referenced below for possible limitations in the earlier studies), two recent studies reported observing such an association.

The first study (Denmark-Wahnefried 2000¹²⁴⁶) provided prostate cancer patients and controls with an illustration of the Hamilton Scale of Baldness and asked participants to select the diagrams that best represent their hair patterning at age 30 and 40. The study collected information from two sources, participants in the Duke-based study (n=149; 78 cases; 71 controls), and participants in the community-based study (n=130; 56 cases; 74 controls). The following table presents the age-adjusted odds ratios (OR) for early and late onset of vertex baldness (Demark-Wahnefried 2000, ibid, from Table 3).

TABLE 27
Observed association between early and late onset
of vertex baldness and prostate cancer.

| | Duke-based study | | | Community-based study | | |
|---|---|---|---|---|---|---|
| | N | OR | 95% CI | N | OR | 95% CI |
| Early onset of | Cases: 10 | 2.11 | 0.66-6.73 | Cases: 6 | 2.44 | 0.57-10.46 |
| vertex baldness | Control: 5 | | | Control: 3 | | |
| (<30 yr. old) | | | | | | |
| Late onset of | Cases: 9 | 1.37 | 0.47-4.06 | Cases: 8 | 2.10 | 0.63-7.00 |
| vertex baldness | Control: 7 | | | Control: 5 | | |
| (<40 yr. old) | | | | | | |

Although the sample sizes are small, results in the community-based study are borderline statistically significant. Based on these observations, Demark-Wahnefried, et al., (2000, ibid) concluded: “the concordance between these results lends strength to our conclusion that early onset vertex baldness may place men at “moderate risk” for prostate cancer.”

A second study (Hawk 2000¹²⁴⁷) used a Cox proportional hazards regression to evaluate the relation between MPA and clinical prostate cancer in a cohort of 4,421 men 25-75 years old without a history of prostate cancer. Participants were followed from baseline (1971-1974) through 1992. Prostate cancer was diagnosed in 214 subjects over 17-21 years of follow-up. The results showed an increase in cumulative incidence of prostate cancer for bald men compared to men with no hair loss (p=0.02). The results also showed greater age-standardized incidence of prostate cancer among men with baldness at baseline (17.5 versus 12.5 per 10,000 person-years). The adjusted relative risk (RR) for prostate cancer among men with baldness was 1.50 (95% CI, 1.12-2.00, p=0.01). RRs were similar after inclusion of additional covariates, such as educational status, region, race, family history of prostate cancer, to the Cox model. RRs were independent of the extent of baldness. Based on these observations, Hawk, et al., (2000, ibid) concluded: “we found a significantly increased risk for prostate cancer among men with MPB, independent of established risk factors.”

Another study (Oh 1998¹²⁴⁸) showed an association between MPA and benign prostatic hyperplasia (BPH). The study compared baldness in 225 BPH patients (mean age 69.3±6.5 years) and 160 controls (mean age 68.5±6.4 years). All subjects were over 60 years old. The results showed higher grade of MPA (median value of grade IV versus III, p<0.001) in BPH patients compared to controls. The proportion of men with grade IV or higher in the BPH group was significantly larger than controls (53.8% vs. 36.9%, p<0.01). The results showed no significant correlation between extent of baldness and International Prostate Symptom Score in either group. Based on these observations, Oh, et al., (1998, ibid) concluded: “this study demonstrates a strong association of BPH with male pattern baldness.”

As expected, MPA is associated with prostate cancer and benign prostatic hyperplasia.

15. Technical Note: Other Disruptions

a) Drug Induced Molecular Disruptions

Microcompetition with foreign DNA disrupts the p300·GABP allocation to cellular genes. Some drugs also disrupt this allocation. As a result, the drugs induce “side effects” similar to the clinical symptoms characteristic of microcompetition with foreign DNA. Some of these side effects are weight gain, insulin resistance, and hypertension. The following sections propose the mechanism underlying these side effects.

(1) Cytochrome P450

Three distinct pathways of arachidonic acid (AA) oxidation have been described. The enzyme systems involved are regiospecific and stereospecific. Of the three pathways, the products of the cyclooxygenase and lipoxygenase pathways have been extensively researched. Research on the products of the “third pathway,” the cytochrome P450-dependent monooxygenases, is less extensive. The “third pathway,” mediated by CYP enzymes, uses NADPH and molecular oxygen in a 1:1 stoichiometry. Three types of oxidative reactions are known to occur. Olefin epoxidation (epoxygenases) produces 4 sets of regioisomers, the epoxyeicosatrienoic acids (EETS), specifically, the (5,6-), (8,9-), (11,12-) and 14,15-EETs. Allylic oxidation produces hydroxyeicosatetraenoic acids (HETEs), specifically, (5-), (8-), (9-), (1]-), (12-) and 15-HETEs. Omega oxidation produces the 19- and 20-HETEs. FIG. 152 presents the three pathways of arachidonic acid oxidation.

(2) Arachidonic Acid Metabolites Activate ERK

A study (Muthalif 1998¹²⁴⁹) treated rabbit VSMCs with the vehicle dimethyl sulfoxide (DMSO) alone or 20 μM PD98059 (PD) for 4 h, and then exposed the cells to 0.25 μM 12(R)-, 12(S)-, 15, or 20-hydroxyeicosatetraenoic acid (HETE) for 10 min. The results showed that 20-HETE specifically activated ERK1 and ERK2 (Muthalif 1998, ibid, FIG. 3D). Wen 1996¹²⁵⁰ and Rao 1994¹²⁵¹ reported similar activation of MAPK by 12-, and 15-HETE. Another study (Chen J K 1999¹²⁵²) tested the effect of 14,15-epoxyeicosatrienoic acid (EET) on ERK activation. LLCPKc14, established proximal tubule epithelial cells derived from pig kidney, were treated with 14,15-EET (20 μm) for 15 min. The results showed that 14,15-EET stimulated ERK1 and ERK2 phosphorylation (Chen J K 1999, ibid, FIG. 2D).

To summarize, 12(S)-, 15, or 20-HETE and 14,15-EET activate ERK. In other words, the arachidonic acid metabolites are ERK agents.

(3) 12(S)—, 15, or 20-HETE and 14,15-EET CYP Enzymes

The following table lists a few cytochrome P450 enzymes that produce metabolites of ERK agents. Call these enzymes CYP-ERKs. When the study is tissue specific, the tissue type is mentioned in the reference column.

TABLE 28
Few cytochrome P450 enzymes that produce
metabolites of ERK agents.

| Enzyme | ERK agent product | Reference* |
|---|---|---|
| CYP1A2 | 14, 15-EET | Rifkind 1995 (human liver) |
| CYP2B4 | 14(R), 15(S)-EET | Zeldin 1995 (lung) |
| CYP2C8 | 14, 15-EET | Rifkind 1995 (human liver) |
| CYP2C9 | 15(R)-HETE | Bylund 1998, |
| | 12-HETE | Rifkind 1995 (human liver) |
| CYP2C19 | 14, 15-EET | Bylund 1998, Keeney 1998 (14S 15R, |
| | | skin keratinocytes) |
| | 12R-HETE | Keeney 1998 (skin keratinocytes) |
| | 15R-HETE | Keeney 1998 (skin keratinocytes) |
| CYP2C23 | 14, 15-EET | Imaoka 1993 (rat kidney) |
| CYP2C29 | 14, 15-EET | Luo 1998 |
| CYP2C39 | 14, 15-EET | Luo 1998 |
| CYP2C37 | 12-HETE | Luo 1998 |

*Bylund 1998¹²⁵³, Imaoka 1993¹²⁵⁴, Zeldin 1995¹²⁵⁵, Rifkind 1995¹²⁵⁶, Luo 1998¹²⁵⁷, Keeney 1998¹²⁵⁸

(4) Inhibition of CYP-ERK and Microcompetition-Like Diseases

Microcompetition with foreign DNA decreases concentration of the p300·GABP·N-box complex of cellular genes. Inhibition of an ERK agent produces the same effect. Consider a drug that only inhibits CYP-ERK. That is, the drug has no other chemical reactions, such as inhibition of another enzyme. Call such a drug an “empty” drug. An empty drug should produce a clinical profile similar to the clinical profile produced by microcompetition with foreign DNA.

The following table lists drugs that inhibit CYP-ERKs and their microcompetition with foreign DNA-like side effects (mostly weight gain, some insulin resistance and atherosclerosis).

TABLE 29
Few drugs that inhibit CYP-ERKs and their microcompetition
with foreign DNA-like side effects.

| | | |
|---|---|---|
| | | Microcompetition-like |
| Drug | Cytochrome P450 (CYP type) | symptoms |

Cytochrome P450 inhibitors

| | | |
|---|---|---|
| Phenytoin | Kidd 1999¹²⁵⁹ (CYP2C9) | Egger 1981¹²⁶² |
| | Ring 1996¹²⁶⁰ (CYP2C9) | |
| | Miners 1998¹²⁶¹ (CYP2C9) | |
| Glipizide | Kidd 1999 (ibid) (CYP2C9) | Campbell 1994¹²⁶³ |
| Carbamazepin | Petersen 1995¹²⁶⁴ (CYP2C9) | Hogan 2000¹²⁶⁶ |
| | Meyer 1996¹²⁶⁵ (through | Mattson 1992¹²⁶⁷ |
| | drug interaction) | |
| Valproic Acid, | Sadeque 1997¹²⁶⁸ (check) | Bruni 1979¹²⁶⁹ |
| Sodium | (CYP2C9) | Egger 1981 (ibid) |
| Valproate | | Zaccara 1987¹²⁷⁰ |
| | | Mattson 1992 (ibid) |
| | | Sharpe 1995¹²⁷¹ |
| Losartan | Song 2000¹²⁷² (CYP2C9) | Camargo 1991¹²⁷⁴ |
| | Meadowcroft 1999¹²⁷³ (CYP2C9) | |
| | Miners 1998 (ibid) (CYP2C9) | |
| Simvastatin | Transon 1996¹²⁷⁵ (CYP2C9) | Matthews 1993^{1276,I} |
| Olanzapine | Ring 1996 (ibid) (CYP2C9) | Osser 1999¹²⁷⁷ |
| | | Koran 2000¹²⁷⁸ |
| Clozapine | Ring 1996 (ibid) (CYP2C9) | Osser 1999 (ibid) |
| | Fang 1998¹²⁷⁹ (CYP2C9) | |
| | Prior 1999¹²⁸⁰ (CYP1A2, CYP2C19) | |
| Fluvoxamine | Olesen 2000¹²⁸¹ (CYP1A2, CYP2C19) | Harvey 2000^{1283,II} |
| Fluoxetine | Miners 1998 (ibid) (CYP2C9) | Sansone 2000¹²⁸⁴ |
| (Prozac) | Schmider 1997¹²⁸² (CYP2C9) | Michelson 1999^{1285,II} |
| | | Darga 1991^{1286,II} |
| Tolbutamide | Ring 1996 (ibid) (CYP2C9) | Wissler 1975^{1288,III} |
| | Miners 1998 (ibid) (CYP2C9) | Ballagi-Pordany 1991^{1289,III} |
| | Lasker 1998¹²⁸⁷ (CYP2C9, CYP2C19) | |
| Anastrozole | Grimm 1997¹²⁹⁰ (CYP1A2, GYP2C9) | Wiseman 1998¹²⁹¹ |
| | | Lonning 1998¹²⁹² |
| | | Buzdar 1998¹²⁹³ |
| | | Jonat 1997¹²⁹⁴ |
| | | Buzdar 1997A¹²⁹⁵ |
| | | Hannaford 1997¹²⁹⁶ |
| | | Buzdar 1997B¹²⁹⁷ |
| | | Buzdar 1996¹²⁹⁸ |
| | | Jonat 1996¹²⁹⁹ |
| Nelfinavir (PI) | Khaliq 2000¹³⁰⁰ (CYP2C19) | VI |
| | Lillibridge 1998¹³⁰¹ | |
| | (CYP2C19, CYP1A2)^{V} | |
| Ritonavir (PI) | Muirhead 2000¹³⁰² (CYP2C9) | VI |
| | Kumar 1999¹³⁰³ (CYP2C9, CYP2C19) | |
| | Kumar 1996¹³⁰⁴ (CYP2C9) | |
| | Eagling 1997¹³⁰⁵ (CYP2C9) | |
| Amprenavir | Fung 2000¹³⁰⁶ (CYP2C9) | VI |
| (PI) | | |
| Saquinavir (PI) | Eagling 1997 (ibid) (CYP2C9) | VI |

Cytochrome P450 inducers

| | | |
|---|---|---|
| Nifedipine | Fisslthaler 2000¹³⁰⁷ (CYP2C9) | Krakoff 1993¹³⁰⁸ |
| | | Maccario 1998¹³⁰⁹ |
| | | Andronico 1991^{1310,IV} |

^{I}Increase in BMI was associated with smaller decrease in common femoral arterial stiffness.
^{II}Fluoxetine produces a transient weight loss leading to gain in body weight in the long term.
^{III}Tolbutamide induced atherosclerosis.
^{IV}Nifedipine decreased insulin resistance.
^{V}Inhibition occurs at supratherapeutic concentrations.
VI Replacing, or not including a protease inhibitor in therapy was associated with attenuated fat distribution abnormalities and insulin resistance (Barreiro 2000¹³¹¹, Mulligan 2000¹³¹², Gervasoni 1999¹³¹³, Carr 2000¹³¹⁴, Martinez 2000¹³¹⁵, see also review, Passalaris 2000¹³¹⁶).

Drugs are not “empty.” Drugs have other chemical reactions aside from inhibition of CYP-ERK. Take a clinical symptom resulting from microcompetition with foreign DNA, such as weight gain. There are three possible events. The other chemical reactions might increase, decrease, or not change body weight. Take the combined effect of CYP-ERK inhibition and the other chemical reactions. The Ho hypothesis assumes a uniform (random) distribution of these events, that is, the probability of every such event is ⅓ so that the probability that a CYP-ERK inhibitor causes weight gain is ⅓. The probability that each of two CYP-ERK different inhibitors cause weight gain is (⅓)*(⅓). In the table above, there are 16 drugs, 15 CYP-ERK inhibitors, and 1 CYP-ERK inducer. The probability that the 15 inhibitors increase weight and the 1 inducer decreases weight, under the H₀ assumption, is (⅓)¹⁶ or <0.0001. Therefore, treatment with CYP-ERK agents shows a statistically significant disruption of body weight.

b) Mutation, Injury, and Diet Induced Disruptions

See chapter on obesity, p 310.

16. Treatment

a) Introduction

(1) Direction

The preceding chapters showed that a decrease in p300·GABP availability to cellular genes increases the rate of disease progression. Let the symbol G denote a cellular gene and [Disease], susceptibility to disease or rate of disease progression. Then,
↓[p300·GABP·N-box_{G}]→↑[Disease]
Sequence of quantitative events 124: Predicted effect of the p300·GABP·N-box_{G} complex on susceptibility to chronic disease, the disruption direction.

This chapter examines the effect of an increase in p300·GABP availability to cellular genes. As expected, the chapter will show that such increase decreases susceptibility to disease or the rate of disease progression. Symbolically,
↑[p300·GABP·N-box_{G}]→↓[Disease]
Sequence of quantitative events 109: Predicted effect of the p300·GABP·N-box_{G} complex on susceptibility to chronic disease, the pretreatment direction.

The chapter is divided into three sections. The first section includes studies with GABP kinase agents. These agents stimulate phosphorylation of a GABP kinase, such as ERK or JNK. The second section includes studies with anti-oxidation agents. The third section includes studies with viral N-box agents. These agents decrease the concentration of viral DNA in the host. All three types of agents increase concentration of p300·GABP·N-box_{G}, and therefore, should decrease the rate of disease progression.

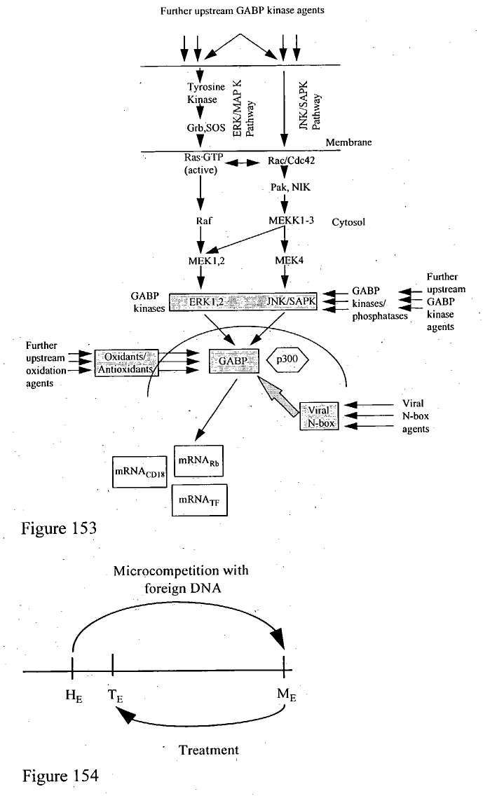

Consider FIG. 153. The targets of the agents are marked with filled boxes. Microcompetition between viral N-box and cellular genes for GABP is marked with a thick arrow.

(2) Magnitude of Change

A healthy system is in stable equilibrium. Microcompetition with foreign DNA establishes a new stable equilibrium, which reflects decreased availability of transcription resources to cellular genes. Assume that the two equilibria are points in a measure space, that is, a space with a unit and direction. In fact, almost all molecular and clinical measurements define such a space. Assume that any point in the space indicates a disease, and that the severity of the disease increases with the distance from the healthy system equilibrium. In this space, the distance between the microcompetition equilibrium and the healthy system equilibrium is small. The small distance between equilibria results in slow progression of the microcompetition disease. Atherosclerosis or cancer, for instance, may take years to become clinically evident. Consider FIG. 154.

Denote the difference between equilibria with Δ, and the difference between the microcompetition equilibrium (M_{E}) and the healthy system equilibrium (H_{E}) with A(M_{E}-H_{E}). Most successful treatments create a new equilibrium (T_{E}) somewhere between M_{E} and H_{E}. The small distance between the microcompetition equilibrium and the healthy system equilibrium poses a challenge in measuring the effectiveness of such treatments. Since T_{E} is between M_{E} and H_{E}, the distance between T_{E} and M_{E} is even smaller than the distance between H_{E} and M_{E}, A(T_{E}-H_{E})<A(M_{E}-H_{E}). Let us assume that the rate of disease progression/regression of the microcompetition diseases is a function of the distance between equilibria. Hence, the decrease in rate of disease progression following treatment is even smaller than the rate of disease progression during microcompetition with foreign DNA. Since the clinical changes induced by the move from point H_{E} to M_{E} are usually difficult to measure, the clinical changes induced by the move from point M_{E} to T_{E} are as difficult, if not more difficult to measure.

To address the issue, the following sections report results of studies that meet two conditions. One, since treatment effectiveness is a reflection of the distance between two states of system equilibria, the following sections mostly include in vivo studies. Second, since the effect of treatment is slow to occur, the following sections only include results of clinical and animal studies conducted over extended periods, at least a few weeks. In some cases, the included studies reported results obtained after years of treatment.

b) GABP Kinase Agents

(1) General Prediction

A GABP kinase agent stimulates phosphorylation of a GABP kinase, such as ERK or JNK. An increase in the GABP kinase phosphorylation increases transcription of GABP stimulated genes and decreases transcription of GABP suppressed genes. Microcompetition with foreign DNA produces the opposite effect on these classes of genes. Therefore, GABP kinase agents should slow the progression of the microcompetition diseases.

Consider a GABP stimulated gene G. Assume a GABP kinase that phosphorylates ERK. Denote the rate of disease progression, or susceptibility to disease with [Disease]. Consider the following sequence of quantitative events. [symbol]
[N-boxᵥ]→ [symbol] [GABP kinase agent]→↑[ERK^{phos}]→^{↑↓[p300·GABP·N-Box}^{_{G}}^{]→}↑↓[mRNA_{G}]→↑↓[Disease]
Sequence of quantitative events 126: Predicted effect of a GABP kinase agent on susceptibility to a microcomposition disease.

The boxed arrows denote the two exogenous events, infection with a GABP virus, and treatment with a GABP kinase agent. The two arrows facing in opposite directions indicate the opposite effect of the two exogenous events on gene transcription and rate of disease progression.

(2) Dietary Fiber

(a) Conceptual Background

(i) Effect on ERK

Dietary fiber produces sodium butyrate, a short chain fatty acid (SCFA), during anaerobic fermentation in the colon. Sodium butyrate is an ERK agent (see above). As a result, sodium butyrate phosphorylates GABP. Symbolically, [symbol]
[Dietary fiber]→↑[Sodium butyrate]→↑[ERK^{phos}]→↑[GABP^{phos}]
Sequence of quantitative events 127: Predicted effect of dietary fiber on GABP phosphorylation.

According to the prediction, consumption of dietary fiber should increase transcription of genes susceptible to microcompetition with foreign DNA, and decrease the rate of disease progression.

(b) Prediction and Observations: Effect on Transcription

(i) Metallothionein (MT)

Microcompetition with a GABP virus decreases MT expression (see chapter on microcompetition, p 128). Therefore, treatment with sodium butyrate should increase MT expression. Symbolically, [symbol]
[Sodium butyrate]→↑[ERK^{phos}]→↑[GABP^{phos}]→↑[p300·GABP·N-Box_{MT}]→↑[mRNA_{MT}]
Sequence of quantitative events 128: Predicted effect of sodium butyrate on metallothionein (MT) mRNA levels.

Consider the following observations. Different embryonic carcinoma cell lines show different basal levels of MT mRNA. For instance, F9 cells show intermediate basal levels of MT expression, while similar PC13 cells show high levels. Since OC15S1 stem cells usually show very low basal levels, a study (Andrews 1987¹³¹⁷) chose the cells for testing the effect of sodium butyrate on MT mRNA. The study treated OC15 embryonic carcinoma cells (OC15 EC), and OC15 cells differentiated in a 4-day culture in presence of retinoic acid (OC 15 END) with sodium butyrate, and measured MT mRNA levels by densitometry of Northern blots. FIG. 155 presents the results (Andrews 1987, ibid, FIG. 1).

As expected, the results showed increased MT mRNA levels in both undifferentiated OC15 EC and differentiated OC15 END cells following treatment with sodium butyrate. F9 EC cells, although having higher MT basal mRNA levels, responded similarly to sodium butyrate treatment. Note that the effect of sodium butyrate was specific since sodium propionate and sodium acetate, the other two products of bacterial fermentation in the colon, showed no effect on MT mRNA levels.

Another study (Thomas 1991¹³¹⁸) used ROS 17/2.8, cloned rat osteosarcoma cells. The results showed a dose-dependent increase in MT synthesis following treatment with sodium butyrate.

A third study (Liu 1992¹³¹⁹) used rat primary non-transformed hepatocytes. Sodium butyrate treatment of these cells produced a 2-4-fold increase in MT mRNA (Liu 1992, ibid, FIG. 6).

Note: Sodium butyrate increased MT mRNA 2-4-fold in the non-transformed hepatocytes (Liu 1992, ibid), and 20-fold in the OC15 carcinoma cells (Andrews 1987, ibid). A possible explanation for the observed difference in MT transactivation is the existence of a higher copy number of foreign DNA in the OC15 cells. The higher copy number may explain the relative low basal level of MT mRNA in these cells, and the larger effect of sodium butyrate in OC15 carcinoma cells relative to the non-transformed hepatocytes (see details on the relation between microcompetition with foreign DNA and cancer in the chapter on cancer, p 358).

The observations in Andrews 1987 (ibid), Thomas 1991 (ibid), and Liu 1992 (ibid) are consistent with the predicted effect of sodium butyrate on a gene susceptible to microcompetition with foreign DNA.

(c) Prediction and Observations: Effect on Clinical Symptoms

(i) Obesity and Insulin Resistance

Dietary fiber consumption should decrease the rate of obesity progression. Dietary fiber should produce a similar effect on insulin resistance. Symbolically, [symbol]
[Dietary fiber]→↑[Sodium butyrate]→↑[ERK^{phos}]→↑[GABP^{phos}]→ . . . →↓[Obesity] and ↓[Insulin resistance]
Sequence of quantitative events 129: Predicted effect of dietary fiber on susceptibility to obesity and insulin resistance.

Consider the following observations. The Coronary Artery Risk Development in Young Adults (CARDIA) Study, a multi-center population-based cohort study, tested the change in cardiovascular disease (CVD) risk factors over a 10-year period (1985-1986 to 1995-1996) in Birmingham, Ala.; Chicago, Ill.; Minneapolis, Minn.; and Oakland, Calif. 2,909 healthy black and white adults, age 18 to 30 years at enrollment, were included in the study. The results showed an inverse relation between consumption of dietary fiber and body weight in both blacks and whites. At all levels of fat intake, subjects consuming the most fiber gained less weight compared to subjects consuming the least fiber. Moreover, the results also showed an inverse relation between fiber consumption, fasting insulin levels, and systolic and diastolic blood pressure, in both black and white subjects (Ludwig 1999¹³²⁰).

A study (Rigaud 1990¹³²¹) enrolled 52 overweight patients with a mean body mass index (BMI) of 29.3 in a 6 month, randomized, double blind, placebo-controlled, parallel group design, study. The treatment included an energy-restricted diet, supplemented with dietary fiber (7 g/day), or placebo. The results showed a significant decrease in body weight in patients treated with fiber compared to patients treated with placebo (5.5±0.7 kg, vs. 3.0±0.5 kg, p=0.005). The fiber treated group also showed a significant decrease in hunger feelings, measured using visual analogue scales (VAS), while the placebo treated group showed a significant increase in hunger feelings (p<0.02).

Another study (Ryttig 1989¹³²²) enrolled 97 mildly obese females in 52 week, randomized, double blind, placebo-controlled trial study. The treatment consisted of a restricted diet (1,200 kcal/day) supplemented with dietary fiber (7 g/day) for 11 weeks, (part I), followed by a richer diet (1,600 kcal/day) supplemented with less dietary fiber (6 g/day) for 16 weeks (part II). Another group was treated with the same diets supplemented with a placebo. At the end of the 25 weeks, all compliant subjects, on fiber and placebo, were given a dietary fiber supplement of 6 g/day and an ad libium diet for the rest of the period (part III). The results showed a significantly larger decrease in body weight, during part I, in the fiber-supplemented group compared to the placebo group (4.9 kg vs. 3.3 kg, respectively, p=0.05). The total decrease in body weight during part I+II remained significantly larger in the fiber-supplemented group compared to the placebo group (3.8 kg vs. 2.8 kg, respectively, p<0.05). The probability of adherence to the diet was significantly higher in the fiber group from week 13 and onwards (p<0.01). The results also showed a significant decrease in systolic blood pressure in both groups. However, only the fiber group showed a significant decrease in diastolic blood pressure (p<0.05).

Note: Average weight loss in the fiber group after 52 weeks was 6.7 kg.

The observations in Ludwig 1999 (ibid), Rigaud 1990 (ibid), and Ryttig 1989 (ibid) are consistent with the predicted effect of dietary fiber on obesity and insulin resistance, both microcompetition diseases (see chapter on obesity, p 310, and chapter on signal resistance, p 340).

(ii) Atherosclerosis

Soybean hull is a rich source of dietary fiber. Therefore, a diet enriched with soybean hull should decrease the rate of atherosclerosis progression. Symbolically, [symbol]
[Soybean hull]→↑[Dietary fiber]→↑[Sodium butyrate]→↑[ERK^{phos}]→↑[GABP^{phos}]→ . . . →↓[Atherosclerosis]
Sequence of quantitative events 130: Predicted effect of soybean hull on susceptibility to atherosclerosis.

Consider the following observations. A study (Piliang 1996¹³²³) divided 25 monkeys into 5 groups. The T1 group received basal diet; T2, basal diet plus palm oil; T3, basal diet plus palm oil and soybean hull; T4, basal diet plus cholesterol, and T5, basal diet plus cholesterol and soybean hull. Water was provided ad lib. The treatment lasted 8 months. At the end of the experiment, the aorta was removed and stained with hematoxylin and cosine. Histopathological observation of the aorta showed that addition of soybean hull to the diet decreased the rate of atherosclerotic lesion formation under basal diet (41.67 vs. 31.25%, for T2 and T3 diets, respectively), and under a cholesterol rich diet (86.25 vs. 53.38%, for T5 and T4 diets, respectively). Based on these observations, Piliang, et al., (1996, ibid) concluded: “the soybean hull given in the diet has the ability to prevent the development of atherosclerosis in the aorta of the experimental animals.”

The results in Piliang 1996 (ibid) are consistent with the predicted effect of soybean hull on atherosclerosis, a microcompetition disease (see chapter on atherosclerosis, p 161).

(iii) Cancer

Cancer is another microcompetition disease. Therefore, dietary fiber should decrease the rate of cancer progression. Symbolically, [symbol]
[Dietary fiber]→↑[Sodium butyrate]→↑[ERK^{phos}]→↑[GABP^{phos}]→ . . . →↓[Cancer]
Sequence of quantitative events 131: Predicted effect of dietary fiber on susceptibility to cancer.

As expected, a number of studies reported an inverse relation between consumption of dietary fiber and risk of several types of cancer (Kim 2000¹³²⁴, Madar 1999¹³²⁵, Camire 1999¹³²⁶, Mohandas 1999¹³²⁷, Heaton 1999¹³²⁸, Cummings 1999¹³²⁹, Ravin 1999¹³³⁰, Reddy 1999A¹³³¹, Reddy 1999B¹³³², Earnest 1999¹³³³, Kritchevsky 1999¹³³⁴, Cohen 1999¹³³⁵).

(3) Acarbose

(a) Conceptual Building Blocks

(i) Effect on Sodium Butyrate

Acarbose is a α-glucosidase inhibitor, a new class of drugs used in treatment of type I and type II diabetes mellitus. α-glucosidases are enzymes released from the brush border of the small intestine. The enzymes hydrolyze di-, and oligosaccharides, derived from diet and luminal digestion of starch by pancreatic amylase, into monosaccharides. Since only monosaccharides are transported across intestinal cell membranes, α-glucosidase inhibition decreases carbohydrate absorption.

Microbial fermentation in the colon produces acetate, propionate, and butyrate. Acarbose inhibits starch digestion in human small intestine, and therefore, increases the concentration of starch available for microbial fermentation. A study (Wolin 1999¹³³⁶) examined fecal suspensions obtained from participants in an acarbose-placebo crossover trial. The results showed 57, 13, and 30% of total short-chain fatty acids for acetate, propionate, and butyrate, respectively, in acarbose treated subjects, and 57, 20, and 23% in placebo treated subjects (Wolin. 1999, ibid, Table 1, p<0.002 for propionate, p<0.02 for butyrate). Based on these observations, Wolin, et al., (1999, ibid) concluded: “our results show that acarbose treatment results in decreases in the activities of colonic bacteria . . . that form propionate and an increase in the activity of bacteria that produce butyrate.” To determine the effects of acarbose on colonic fermentation, the study treated subjects with 50-200 mg acarbose, or placebo (cornstarch), three times per day, with meals, in a double-blind crossover study. Fecal concentrations of starch and starch-fermenting bacteria were measured, and fecal fermentation products were determined after incubation of fecal suspensions with and without added substrate for 6 and 24 h. Substrate additions were cornstarch, cornstarch plus acarbose, and potato starch. Dietary starch consumption was similar during acarbose and placebo treatment periods. The results showed significantly more butyrate in feces, measured as absolute concentration or percentage of total short-chain fatty acids, following treatment with acarbose compared to placebo, and significantly less propionate (Wolin 1999, ibid, Table 1, p<0.0001). Moreover, samples collected during acarbose treatment showed increased production of butyrate, and decreased production of acetate and propionate, during in vitro fermentations. Based on their results, Wolin, et al., (1999, ibid) concluded: “acarbose effectively augmented colonic butyrate production by several mechanisms; it decreased starch absorption, expanded concentrations of starch-fermenting and butyrate-producing bacteria, and inhibited starch use by acetate- and propionate-producing bacteria.”

Sodium butyrate is an ERK agent. Therefore, acarbose should increase ERK and GABP phosphorylation. Symbolically, [symbol]
[Acarbose]→↑[Sodium butyrate]→↑[ERK^{phos}]→↑[GABP^{phos}]
Sequence of quantitative events 132: Predicted effect of acarbose on GABP phosphorylation.

(b) Prediction and Observations: Effect on Clinical Symptoms

(i) Obesity

Treatment with acarbose should decrease the rate of obesity progression. Symbolically, [symbol]
[Acarbose]→↑[Sodium butyrate]→↑[ERK^{phos}]→↑[GABP^{phos}]→ . . . →↓[Obesity]
Sequence of quantitative events 133: Predicted effect of acarbose on susceptibility to obesity.

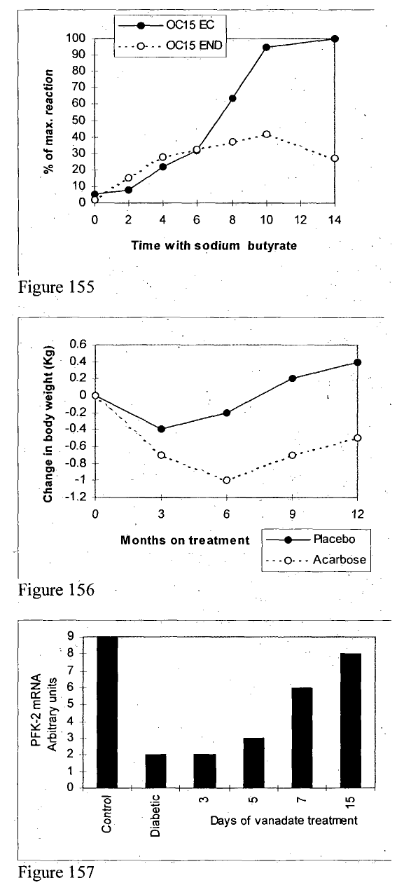

Consider the following observations. A study (Wolever 1997¹³³⁷) treated non-insulin-dependent diabetes (NIDDM) patients with acarbose or placebo for 1 year in a randomized, double blind, placebo-controlled, parallel design study. FIG. 156 presents the effect of acarbose treatment on body weight (Wolever 1997, ibid, FIG. 1).

After one year, the 130 subjects treated with acarbose showed an average weight loss of 0.46±0.28 kg. In contrast, the 149 subject treated with placebo showed a weight gain of 0.33±0.25 kg (p=0.027). Acarbose had no effect on energy intakes, nutrient intakes, or dietary patterns.

The observations in Wolever 1997 (ibid) are consistent with the predicted effect of acarbose on obesity, a microcompetition disease (see chapter on obesity, p 310).

(4) Vanadate

(a) Conceptual Building Blocks

(i) Introduction

An ERK phosphatase is an enzyme that inactivates ERK by dephosphorylation of either Thy, Tyr, or both residues (see chapter on signal resistance, p 340). The class of ERK phosphatases includes PP2A, a type 1/2 serine/threonine phosphatase, PTP1B, a protein tyrosine phosphatase, and MKP-1, a dual specificity phosphatase. Inhibition of an ERK phosphatase stimulates ERK phosphorylation. The increase in ERK phosphorylation increases transcription of GABP stimulated genes and decreases transcription of GABP suppressed genes. Since, microcompetition with foreign DNA has the opposite effect on these classes of genes, inhibition of an ERK phosphatase decreases the rate of microcompetition disease progression.

(ii) Effect on PTP

Vanadate (VO₄⁻³) and vanadate derivatives are general protein tyrosine phosphatase (PTP) inhibitors. Specifically, a study (Huyer 1997¹³³⁸) showed inhibition of the protein-tyrosine phosphatase PTP1B by vanadate and pervanadate (a general term for the variety of complexes formed between vanadate and hydrogen peroxide).

(iii) Effect on ERK

PTPs dephosphorylate and deactivate ERK (see above). As general PTP inhibitors, vanadate and vanadate derivatives activate ERK (Wang 2000¹³³⁹, Zhao 1996¹³⁴⁰, Pandey 1995¹³⁴¹, D'Onofrio 1994¹³⁴²), and therefore, should increase GABP phosphorylation. Symbolically, [symbol]
[Vanadate]→↓[PTP]→↑[ERK^{phos}]→↑[GABP^{phos}]
Sequence of quantitative events 134: Predicted effect of vanadate on GABP phosphorylation.

(b) Prediction and Observations: Effect on Genes

(i) F-type PFK-2/FBPase-2 is GABP Stimulated Gene

The F-type PFK-2/FBPase-2 is a GABP stimulated gene. Consider the following observations. The bifunctional enzyme 6-phosphofructo-2-kinase (EC 2.7.1.105, PFK-2)/fructose-2,6-bisphosphatase (EC 3.1.3.46 FBPase-2) catalyzes the synthesis and degradation of fructose-2,6-bisphosphate. The rat PFK-2/FBPase-2 gene (gene A) codes for the fetal (F), muscle (M), and liver (L) mRNA. Each of these mRNA forms originates from a different promoter. The F-type promoter includes an enhancer in the (−1809-1615) region with three N-boxes at (−1747, −1742), (−1716, −1710), and (−1693, −1688) (Darville 1992¹³⁴³, FIG. 4). The enhancer stimulated transcription, especially in FTO2B hepatoma cells (Darville 1992, ibid, Table 1). DNase I protection experiments using the enhancer and extracts from FTO2B cell, from C2C12 myoblasts or myocytes, or from liver, but not from muscle, showed one specific footprint corresponding to the middle N-box (Darville 1992, ibid, FIG. 5). Gel retardation assays with extracts from FTO2B and HTC cells, L6 myoblasts and myocytes, and liver, but not muscle, showed a major complex (Darville 1992, ibid, FIG. 6A). When the enhancer fragment was methylated at single purines using dimethylsulfate and subsequently incubated with FTO2B extracts, three contact points were detected within the N-box (Darville 1992, ibid, FIG. 4). The three points of methylation interference coincide with contact points identified by the same technique in the two N-boxes of the adenovirus E1A core enhancer that binds GABP. A subsequent study (Dupriez 1993¹³⁴⁴) showed that changing the GG, essential for ETS DNA binding, to CC in both distal and proximal N-boxes decreased promoter activity by 15-20%. Changing GG to CC in the middle N-box decreased promoter activity by 75%. The study also showed that anti-GABPα and anti-GABPβ antibodies inhibited formation of complexes on the middle N-box by FTO2B proteins (Dupriez 1993, ibid, FIG. 4, lane 5 and 6). Transfection with recombinant GABPα and GABPβ produced shifts that co-migrated with these complexes and were inhibited by anti-GABPα antibodies (Dupriez 1993, ibid, FIG. 4, lane 12-16). These observations suggest that the F-type PFK-2/FBPase-2 is a GABP stimulated gene.

(ii) Transcription of F-type PFK-2/FBPase-2

Rat F-type PFK-2/FBPase-2 gene is a GABP stimulated gene. Therefore, vanadate should stimulate transcription of F-type PFK-2/FBPase-2. Symbolically, [symbol]
[Vanadate]→↓[PTP]→↑[ERK^{phos}]→↑[GABP^{phos}]→↑[p300·GABP·N-Box_{F-type PFK-2}]→↑[mRNA_{F-type PFK-2}]
Sequence of quantitative events 135: Predicted effect of vanadate on mRNA levels of the rat F-type PFK-2/FBPase-2 gene.

Consider the following observations. A study (Miralpeix 1992¹³⁴⁵) measured the effect of treatment with sodium orthovanadate on liver PFK-2/FBPase-2 mRNA content of rats with streptozotocin (STZ)-induced diabetes. mRNA content was measured 3, 5, 7 and 15 days post treatment. FIG. 157 presents the results (Miralpeix 1992, ibid, FIG. 3).

Vanadate treatment of diabetic animals produced a progressive increase in liver PFK-0.2/FBPase-2 mRNA content, reaching nearly normal levels after 15 days. Inoue 1994¹³⁴⁶ reports similar observations.

Note that Miralpeix 1992 (ibid) used a “1.4 kilobase rat liver PFK-2/FBPase-2 cDNA probe which corresponds to the mRNA for liver PFK-2/FBPase-2 devoid of the 5′ end coding for amino acids 1-90.” The probe does not distinguish between F-type and L-type PFK-2/FBPase-2 mRNA. Therefore, the question is what type of gene showed an increase in mRNA following treatment with sodium orthovanadate, the F-type, which is a GABP stimulated gene, or L-type? To answer the question, we need to combine observations from several studies.

First, consider Dupriez 1993 (ibid), which measured expression of the PFK-2/FBPase-2 gene in various tissues. The observation showed expression of F-type PFK-2/FBPase-2 mRNA in hepatoma, fibroblast, and myoblasts cell lines. Expression Was also found in fetal liver and muscle, the only two fetal tissues examined. In adult tissues, F-type PFK-2/FBPase-2 mRNA was found in the lung and thymus. In the other adult tissues tested, the mRNA was present at much lower concentrations or was undetectable. The highest concentration was in preterm placenta, with a decrease at term. The concentration decreased upon differentiation of L6 myoblasts into myocytes (Dupriez 1993, ibid, FIG. 2), and in Rat-1 fibroblasts made quiescent by lowering serum concentration in culture from 10 to 0.1%. Moreover, F-type mRNA concentration increased in FTO2B cells upon dexamethasone treatment. Based on these observations, Dupriez, et al., (1993, ibid) concluded: the “expression of the F-type mRNA appears to correlate with cell proliferation.”

Usually, liver tissue shows limited cell proliferation. However, in Miralpeix 1992 (ibid), vanadate was administered to male Sprague-Dawley rats one week after the animals were treated with a single intravenous injection of streptozotocin (STZ). As it turns out, STZ injection to Sprague-Dawley rats induces high levels of hepatocyte proliferation. Consider the following observations.

A study (Herrman 1999¹³⁴⁷) measured hepatocyte proliferation in Sprague-Dawley rats made diabetic by IV injection of STZ. The results showed a 12% increase in ratio of liver weight to body weight in diabetic rats 8 days after injection compare to normal rats, and a 44% increase at 30 days. The results also showed an increase in hepatocyte mitosis to 300% of normal at 8 days, a return to normal at 30 days, and a decrease to 25% of normal at 90 days (Herrman 1999, ibid, FIG. 1). Based on these results, Herrman, et al., (1999, ibid) concluded: “hepatomegaly observed in streptozotocin-induced experimental diabetes may be due primarily to early hyperplasia.”

The combined observations in Dupriez 1993 (ibid) and Herrman 1999 (ibid) suggest that although the probe used in Miralpeix 1992 (ibid) does not distinguish between F-type and L-type PFK-2/FBPase-2 mRNA, the gene that showed an increase in expression following treatment with sodium vanadate (Miralpeix 1992, ibid) is, most likely, the F-type, a GABP stimulated gene. The streptozotocin injection in Miralpeix 1992 (ibid) increased hepatocyte proliferation and mRNA levels of the F-type PFK-2/FBPase-2 gene, which were further increased by treatment with sodium orthovanadate.

Note that the PFK-2/FBPase-2 in controls (see figure above) is probably the L-type (liver type).

The observations in Miralpeix 1992 (ibid) are consistent with the predicted effect of sodium orthovanadate on transcription of the F-type PFK-2/FBPase-2, a GABP stimulated gene.

(c) Prediction and Observations: Effect on Clinical Symptoms

(i) Obesity

Treatment with vanadate should decrease the rate of obesity progression. Symbolically, [symbol]
[Vanadate]→↓[PTP]→↑[ERK^{phos}]→↑[GABP^{phos}]→ . . . →↓[Obesity]
Sequence of quantitative events 136: Predicted effect of vanadate on susceptibility to obesity.

A study (Pugazhenthi 1995¹³⁴⁸) treated 5 week-old Zucker rats (6 animals), an animal model of obesity and insulin resistance, with sodium orthovanadate delivered through drinking water for 4 months. The results showed a 43% decrease in body weight levels compared to untreated obese (fa/fa) controls (6 animals). At the end of the experiment, the treated rats showed body weight levels comparable to lean (Fa/fa) control (6 animals) (Pugazhenthi 1995, ibid, Table 1).

Another study (McNeill 1996¹³⁴⁹) treated Wistar rats (11 animals) with bis(maltolato)oxovanadium (0.3-0.5 mmol/kg/day) delivered in drinking water over a 77 day period. Beginning at day 56, the treated animals showed decreased weight gain compared to controls (8 animals) (McNeill 1996, ibid, FIG. 1, group 2 vs. group 1). (See also Dai 1994¹³⁵⁰, and Bhanot 1994¹³⁵¹.)

The observations in Pugazhenthi 1995 (ibid) and McNeill 1996 (ibid) are consistent with the predicted effect of vanadate on obesity, a microcompetition disease.

(i) Cancer

Treatment with vanadate should decrease the rate of cancer progression. Symbolically, [symbol]
[Vanadate]→↓[PTP]→↑[ERK^{phos}]→↑[GABP^{phos}]→ . . . →↓[Cancer]
Sequence of quantitative events 137: Predicted effect of vanadate on susceptibility to cancer.

A study (Cruz 1995¹³⁵²) tested the antineoplastic effect of orthovanadate on a subcutaneous MDAY-D2 tumor mouse model. Ten week old DBA/2j female mice were injected subcutaneously in the posterior lateral side with 4×10⁵ cells in 100 μl of PBS. On day 5, the mice were divided into two groups. One group received subcutaneous injections of 100 μl of PBS and the other group received 100 μl of PBS containing 500 μg of orthovanadate daily. The orthovanadate was administrated subcutaneously on the opposite, tumor-free, posterior lateral side. On day 14, the mice were sacrificed, weighed and tumors were resected and weighed. The results showed decreased tumor growth in treated mice compared to controls (Cruz 1995, ibid, FIG. 6). In control mice, the tumor weights varied from 0.86-1.74 g, whereas in orthovanadate treated mice, four mice showed no detectable tumors, and 11 mice showed tumors varying from 0.08-0.47 g. Orthovanadate treatment decreased tumor growth by more than 85%, sometimes completely inhibiting tumor formation.

Another study (Bishayee 1995¹³⁵³) tested the chemoprotective effect of vanadium against chemically induced hepatocarcinogenesis in rats. A single intraperitoneal injection of diethylnitrosamine (DENA; 200 mg kg⁻¹) was used to induce tumors, and phenobarbital (0.05%) in diet to promote tumor growth. Vanadium (0.5 ppm) was provided ad libium throughout the experiment in drinking water. The results showed a decrease in incidence (p<0.01), total number, and multiplicity (p<0.001), and altered distribution of the size of visible persistent nodules (PNs), after 20 weeks in vanadium treated animals compared to controls. Mean nodular volume (p<0.05), and nodular volume as a percent of liver volume (p<0.01), was also decreased. Vanadium also decreased the number (p<0.001), and surface area (p<0.01) of gamma-glutamyltranspeptidase (GGT)-positive hepatocyte foci, and decreased the labeling index (p<0.001) of focal cells. Vanadium also decreased activity of GGT in PNs and non-nodular surrounding parenchyma of treated rats (p<0.01). Histopathological analysis of liver sections showed well-maintained hepatocellular architecture in treated animals compared to control. Based on these observations, Bishayee and Chatterjee (1995 ibid) concluded: “our results, thus, strongly suggest that vanadium may have a unique anti-tumor potential.” See also Liasko 1998¹³⁵⁴.

The observations in Cruz 1995 (ibid), Bishayee 1995 (ibid), and Liasko 1998 (ibid) are consistent with the predicted effect of vanadate on cancer, a microcompetition disease.

(iii) Insulin Resistance and Hyperinsulinemia

Vanadate should decrease insulin resistance and hyperinsulinemia. Symbolically, [symbol]
[Vanadate]→↓[PTP]→↑[ERK^{phos}]→↑[GABP^{phos}]→ . . . →↓[Insulin resistance] and ↓[Hyperinsulinemia]
Sequence of quantitative events 138: Predicted effect of vanadate on susceptibility to insulin resistance and hyperinsulinemia.

As expected, numerous in vivo studies demonstrated decreased blood glucose in insulin deficient diabetic animals, and improved glucose homeostasis in obese, insulin-resistant diabetic animals, following treatment with vanadate. In human studies, insulin sensitivity improved in NIDDM patients and in some IDDM patients after treatment with vanadate (see reviews Goldfine 1995¹³⁵⁵, Brichard 1995¹³⁵⁶)

As example, consider Pugazhenthi 1995 (ibid, see above). The study also tested the effect of vanadate on hyperinsulinemia. The obese Zucker rats showed elevated plasma levels of glucose and insulin. Vanadate treatment decreased plasma glucose and insulin levels by 36% and 80%, respectively (Pugazhenthi 1995, ibid, Table 1).

(5) PTP1B Gene Disruption

(a) Conceptual Building Blocks

(i) Effect on PTP and ERK

Gene disruption is a specific case of an exogenous event. PTP1B gene disruption results in PTP1B enzyme deficiency. Vanadate inhibits PTP1B (Huyer 1997, ibid). Therefore, disruption of PTP1B and administration of vanadate both decrease activity of PTP1B. Considering the discussion above, the effects of a PTP1B gene disruption on ERK and GABP phosphorylation should be similar to the effects of vanadate treatment. Symbolically, [symbol]
PTP1B(−/−)→↓[PTP1B]→↑[ERK^{phos}]→↑[GABP^{phos}]
Sequence of quantitative events 139: Predicted effect of PTP1B gene disruption on GABP phosphorylation.

(b) Prediction and Observations: Effect on Clinical Symptoms

(i) Obesity

PTP1B gene disruption should decrease the rate of obesity progression. Symbolically, [symbol]
PTP1B(−/−)→↓[PTP1B]→↑[ERK^{phos}]→↑[GABP^{phos}]→ . . . →↓[Obesity]
Sequence of quantitative events 140: Predicted effect of PTP1B gene disruption on susceptibility to obesity.

A study (Elchebly 1999¹³⁵⁷) generated transgenic mice by replacing exon 5 and the tyrosine phosphatase active site in exon 6 of the mouse homolog of the PTP1B gene with the neomycin resistance gene. The study then microinjected two separate embryonic stem cell clones, which showed single integration following homologous recombination, into Balb/c blastocytes. Chimeric males were mated with wild-type Balb/c females, and heterozygotes from the cross were mated to product animals homozygous for the PTP1B mutation (Elchebly 1999, ibid, FIG. 1A). PTP1B null mice (PTP1B(−/−)) showed no PTB1B protein, and heterozygotes (PTP1B(+/−)) showed about one half the wild-type expression levels (Elchebly 1999, ibid, FIG. 1B). PTP1B null mice grew normally on regular diet, showed similar weight gain compared to wild-type, lived longer than 1.5 years without signs of abnormality, and were fertile. To examine the effect of PTP1B gene disruption on obesity, the study fed PTP1B(−/−), PTP1B(+/−), and wild-type mice a high-fat diet for 10 weeks. The results showed a diminished increase in body weight in PTP1B(−/−) and PTP1B(+/−) compared to wild-type mice (Elchebly, ibid, FIG. 5). Based on these results, Elchebly, et al., (1999, ibid) concluded that PTP1B deficiency results in obesity resistance.

Another study (Klaman 2000¹³⁵⁸) reported results of a PTP1B gene disruption. The study generated PTP1B-null mice by targeted disruption of the ATG coding exon (exon 1). The PTP1B-deficient mice showed low adiposity and protection from diet-induced obesity. The decreased adiposity resulted from decreased fat cell mass with no decrease in adipocyte number. Leanness in PTP1B-deficient mice was associated with increased basal metabolic rate and total energy expenditure.

The observation in Elchebly 1999 (ibid) and Klaman 2000 (ibid) are consistent with the predicted effect of PTP1B gene disruption on obesity, a microcompetition disease.

(ii) Insulin Resistance and Hyperinsulinemia

PTP1B gene disruption should decrease insulin resistance and hyperinsulinemia. Symbolically, [symbol]
PTP1B(−/−)→↓[PTP1B]→↑[ERK^{phos}]→↑[GABP^{phos}]→ . . . →↓[Insulin resistance] and ↓[Hyperinsulinemia]
Sequence of quantitative events 141: Predicted effect of PTP1B gene disruption on susceptibility to insulin resistance and hyperinsulinemia.

Elchebly 1999 (ibid) also tested the effect of PTP1B gene disruption on insulin resistance. Fed PTP(−/−) mice on a regular diet showed a 13% decrease, and PTP(+/−) mice a 8% decrease in blood glucose concentration relative to wild-type mice (Elchebly 1999, ibid, FIG. 2A). Fed PTP1B(−/−) mice on regular diet also showed a decrease in circulating insulin levels to about one half of wild-type fed animals (Elchebly 1999, ibid, FIG. 2B). Increased insulin sensitivity of PTP 1 B(−/−) mice was also observed in glucose and insulin tolerance tests (Elchebly 1999, ibid, FIGS. 3A and 3B). The study also fed the PTP1B(−/−), PTP1B(+/−), and wild-type mice a high-fat diet. The wild-type mice became insulin resistant. In contrast, the PTP1B(−/−) mice showed glucose and insulin concentrations similar to animals on regular diet (Elchebly 1999, ibid, Table 1). PTP1B(−/−) mice on high-fat diet also showed increased insulin sensitivity relative to wild-type in both glucose and insulin tolerance tests (Elchebly 1999, ibid, FIG. 6A, 6B). On high-fat diet, the PTP1B(+/−) mice showed increased fasting concentrations of circulating insulin but similar fasting glucose concentrations relative to animals on regular diet (Elchebly 1999, ibid, Table 1). Based on these results, Elchebly, et al., (1999, ibid) concluded that a decrease in PTP1B expression increases insulin sensitivity.

The PTP1B-deficient mice in Klaman 2000 (ibid) showed a similar increase in insulin-stimulated whole-body glucose disposal.

The observations in Elchebly 1999 (ibid) and Klaman 2000 (ibid) are consistent with the predicted effect of PTP1B gene disruption on insulin resistance and hyperinsulinemia.

Note: It is reasonable to conclude that a disruption of the PTP1B gene will induce resistance to cancer in a manner similar to treatment with vanadate.

c) Antioxidants

(1) General Prediction

Microcompetition with foreign DNA and oxidative stress both decrease formation of the GABP·N-box complex. Therefore, microcompetition with foreign DNA can be viewed as “excessive oxidative stress.” Some antioxidants decrease intracellular oxidative stress. These antioxidants stimulate binding of GABP to the N-box, thereby attenuating the effect of microcompetition with foreign DNA on cellular gene transcription, which decreases the rate of microcompetition disease progression. Symbolically, [symbol]
[N-boxᵥ]→ [symbol] [Antioxidant]→^{↑↓[p300·GABPN-Box}^{_{G}}^{]→↑↓[Disease]}
Sequence of quantitative events 142: Predicted effect of antioxidant treatment on microcompetition disease.

The boxed arrows denote the two exogenous events, infection with a GABP virus, and treatment with an antioxidant. The two arrows facing in opposite directions indicate the opposite effect of the two exogenous events on formation of the p300·GABP·N-Box_{G} complex, gene transcription, and rate of disease progression.

Note: A study (Ojuka 2003¹³⁵⁹) recently showed an increase of GABP binding to the N-box of the cytochrome oxidase subunit IV promoter following treatment with caffeine, an agent which increases cytosolic calcium (Ojuka 2003, ibid, FIG. 3B, D, E). Moreover, exposure to dantrolene, which blocks Ca²⁺ release from the sarcoplasmic reticulum (SR), prevented the effect. Consider the following sequence of quantitative events. [symbol]
[N-Boxᵥ]→ [symbol] [Caffeine]→^{↑↓[p300·GABP·N-Box}^{_{G}}^{]→↑↓[mRNA}^{_{G}}^{]→↑↓[Disease]}
Sequence of quantitative events 143: Predicted effect of caffeine on microcompetition disease.

Another study (Baar 2002¹³⁶⁰) showed a similar increase of GABP binding to the N-box of the cytochrome oxidase subunit IV promoter following a bout of exercise (Baar 2002, ibid, FIG. 4B, C). Consider the following sequences of quantitative events. [symbol]
[N-Boxᵥ]→ [symbol] [Exercise]→^{↑↓[p300·GABP·N-Box}^{_{G}}^{]→↑↓[mRNA}^{_{G}}^{]→↑↓[Disease]}
Sequence of quantitative events 144: Predicted effect of exercising on microcompetition disease.

These sequences of quantitative events might explain the observed protective effect of caffeine and exercising against several microcompetition diseases.

(2) Garlic

(a) Conceptual Building Blocks

(i) Effect on Oxidative Stress

A study (Prasad 1996¹³⁶¹) investigated the ability of unheated or heated garlic extract to scavenge hydroxyl radical (•OH) generated by photolysis of H₂O₂ (1.2-10 μmoles/ml) with ultraviolet (UV) light and trapped with salicylic acid (500 nmoles/ml). H₂O₂ produced •OH in a concentration-dependent manner as estimated by the •OH adduct products 2,3-dihydroxybenzoic acid (DHBA) and 2,5-DHBA. Garlic extract (5-100 μl/ml) inhibited (30-100%) 2,3-DHBA and 2,5-DHBA production in a concentration-dependent manner (Prasad 1996, ibid, FIG. 3). Heating to 100° C. for 20, 40, or 60 min decreased garlic activity by about 10%. Garlic extract also prevented the •OH-induced formation of malondialdehyde (MDA) in rabbit liver homogenate in a concentration-dependent manner (Prasad 1996, ibid, FIG. 10). In absence of •OH, garlic did not affect MDA levels. Based on these results, Prasad, et al., (1996, ibid) concluded: “garlic extract is a powerful scavenger of •OH.”

Another study (Ide 1999¹³⁶²) examined the antioxidant effect of garlic extract in a cellular system using bovine pulmonary artery endothelial cells (PAEC) and murine macrophages (J774). The study used intracellular glutathione (GSH) depletion as an index of oxidative stress. Oxidized LDL (Ox-LDL) depleted GSH. Pretreatment with, aged garlic extract inhibited the Ox-LDL induced peroxides in PAEC, and suppressed peroxides in macrophages in a dose-dependent manner. In a cell free system, aged garlic extract showed similar scavenging activity of H₂O₂. The observations indicate that aged garlic extract can prevent the Ox-LDL-induced depletion of GSH in endothelial cells and macrophages.

The following symbolic presentation summarizes the observations in Prasad 1996 (ibid) and Ide 1999 (ibid).
↑[Garlic]→↓OS
Sequence of quantitative events 145: Predicted effect of garlic on oxidative stress (OS).

(b) Predictions and Observations: Effect on Clinical Symptoms

(i) Atherosclerosis

Garlic is an antioxidant in macrophages. Therefore, treatment with garlic should decrease the rate of atherosclerosis progression. Symbolically, [symbol]
[Garlic]→↓OS→↑[p300·GABP·N-box_{G}]→ . . . →↓[Atherosclerosis]
Sequence of quantitative events 146: Predicted effect of garlic on susceptibility to atherosclerosis.

Consider the following observations. A study (Efendy 1997¹³⁶³) induced de-endothelialization of the right carotid artery of 24 rabbits by balloon catheterization. After 2 weeks, the study randomly assigned the rabbits to receive four diets: standard diet (Group I), standard diet supplemented with 800 μl/kg body weight/day of the aged garlic extract “Kyolic” (Group II), a standard diet supplemented with 1% cholesterol (Group III), and standard diet supplemented with 1% cholesterol and Kyolic (Group IV). After 6 weeks, rabbits on the cholesterol-rich diet (Group III) showed a 6-fold increase in serum cholesterol levels compared to rabbits on standard diet (Group I) (p<0.05) (Efendy 1997, ibid, FIG. 1). The rabbits on the cholesterol-rich diet (Group III) also showed fatty streak lesions covering approximately 70±8% of the surface area of the thoracic aorta. Rabbits on standard diet showed no lesions (Group I and II). Rabbit on the cholesterol-rich+Kyolic diet (Group IV) showed fatty lesions in 25±3% of the same surface area (Efendy 1997, ibid, FIG. 2A and 2B), representing a decrease of about 64% in lesion area compared to rabbits on cholesterol-rich diet without Kyolic (Group III). Rabbits on the cholesterol-rich diet+Kyolic (Group IV) also showed decreased aortic arch cholesterol compared to rabbits on cholesterol-rich diet without Kyolic (Group III) (1.7±0.2 vs. 2.1±0.1 mg cholesterol/g tissue, p<0.05). Kyolic also significantly decreased the size of the neointima (23.8±2.3% vs. 42.6±6.5%, intima+media as percent of artery wall in Group IV vs. Group III, respectively, p<0.01). Kyolic showed little effect in rabbits on a standard diet. Based on these results, Efendy, et al., (1997 ibid) concluded: “Kyolic treatment decreases fatty streak development, vessel wall cholesterol accumulation and the development of fibro fatty plaques in neointimas of cholesterol-fed rabbits, thus providing protection against the onset of atherosclerosis.”

Jain (1978¹³⁶⁴), Jain (1976¹³⁶⁵), and Bordia (1975¹³⁶⁶) reported similar observations. Jain 1978 (ibid) and Jain 1976 (ibid) used rabbits on a 16-week standard or cholesterol-rich diet supplemented with or without garlic extract. In both studies, the results showed marked atherosclerotic lesions in animals fed a cholesterol-rich diet relative to standard diet. The animals on a cholesterol-rich diet supplemented with garlic extract showed decreased rate of lesion formation. Jain 1978 (ibid) also reports decreased aorta cholesterol content in garlic treated animals. Bordia 1975 (ibid) used rabbits fed similar diets for 3 months. The results showed a decreased rate of atherosclerotic plaque formation and decreased lipid content in aorta of rabbits on a diet supplemented with garlic extract.

Garlic treatment resulted in other favorable effects associated with attenuated atherosclerosis. A study (Breithaupt-Grogler 1997¹³⁶⁷) measured the elastic properties of the aorta using pulse wave velocity (PWV), and pressure-standardized elastic vascular resistance (EVR) techniques. The subjects included healthy adults (n=101; age 50 to 80 years) treated with standardized garlic powder (300 mg/d or more) for at least 2 years and 101 age- and sex-matched controls. The two groups showed similar levels of blood pressure, heart rate, and plasma lipids. The results showed a significant decrease in PWV (8.3±1.46 vs. 9.8±2.45 m/s; p<0.0001) and EVR (0.63±0.21 vs. 0.9±0.44 m²·s²·mm Hg⁻¹; p<0.0001) in the garlic compared to control group (Breithaupt-Grogler 1997, ibid, Table 1, FIG. 1). Regression analysis demonstrated that age and SBP are the most important determinants of PWV, and that an increase in age or SBP increases PWV. The garlic treated group showed an attenuated effect of age and SBP on PWV (p<0.0001) (Breithaupt-Grogler 1997, ibid, FIG. 3, FIG. 4). Based on these observations, Breithaupt-Grogler, et al., (1997, ibid) concluded: “The data suggested that the elastic properties of the aorta were maintained better in the garlic group than in the control group.”

Note: In experimental animals, changes in the ratio of intimal to medial area during progression and regression of atherosclerosis showed a positive relation with changes in indices of aortic elastic properties. Progression of atherosclerosis increased PWV, and regression decreased PWV (Farrar 1991¹³⁶⁸)

See also studies in the special supplement of the British Journal of Clinical Practice (1990, Supplement 69) dedicated to the clinical effects of garlic in ischemic heart disease.

The observations in Efendy 1997 (ibid), Jain 1978 (ibid), Jain 1976 (ibid), Bordia 1975 (ibid), and Breithaupt-Grogler 1997 (ibid) are consistent with the predicted effect of garlic on atherosclerosis, a microcompetition disease.

(ii) Cancer

Garlic should decrease susceptibility to cancer, and rate of cancer progression. Symbolically, [symbol]
[Garlic →↓OS →↑[p300·GABP·N-box_{G}]→ . . . →↓[Cancer]
Sequence of quantitative events 147: Predicted effect of garlic on susceptibility to cancer.

The anticancer properties of garlic were recognized thousands of years ago. The ancient Egyptians used garlic externally for treatment of tumors. Hippocrates and physicians in ancient India are also reported to have used garlic externally for cancer treatment. Recent studies confirmed these properties. See, for instance, the section “Garlic, Onions and Cancer,” in Ali 2000¹³⁶⁹, a recent review, the meta-analysis of the epidemiologic literature on garlic consumption and the risk of stomach and colon cancer (Fleischauer 2000¹³⁷⁰), and specific animals studies demonstrating garlic suppression of chemically induced tumors (Singh A 1998¹³⁷¹, Singh 1996¹³⁷²).

d) Viral N-Box Agents

(1) General Prediction

A viral N-box agent decreases the number of active viral N-boxes in the host cell nucleus. The decrease can be accomplished by an overall decrease in the copy number of viral genomes present in the nucleus, or by inhibition of viral N-boxes (for instance by antisense). The decreased number of active viral N-boxes eases microcompetition and consequently decreases susceptibility or slows progression of the microcompetition diseases. Symbolically, [symbol]
[Antiviral agent]→↓[N-boxᵥ]→↑[p300·GABP·N-Box_{G}]→ . . . →↓[Disease]
Sequence of quantitative events 148: Predicted effect of an antiviral agent on microcompetition disease.

The boxed arrows denote the two exogenous events, infection with a GABP virus, and treatment with an antiviral agent. The two arrows facing in opposite directions indicate the opposite effect of the two exogenous events on formation of the p300·GABP·N-box_{G} complex, gene transcription, and rate of disease progression.

(2) Direct Antiviral Agents

(a) Ganciclovir

(i) Effect on Viral DNA Elongation

Ganciclovir (Cytovene, DHPG) is a guanosine analogue. The prodrug is phosphorylated by thymidine kinase to the active triphosphate form after uptake into infected cell. The triphosphate form inhibits viral DNA polymerase by competing with cellular deoxyguanosine triphosphate for incorporation into viral DNA causing chain termination. Ganciclovir is effective against herpes simplex virus 1 and 2 (HSV-1, HSV-2), cytomegalovirus (CMV), Epstein-Barr virus (EBV), and varicella-zoster virus (Spector 1999¹³⁷³).

Aciclovir (acyclovir) and its oral form valacyclovir, and penciclovir, and it oral form famciclovir, are guanosine analogues similar to ganciclovir. These drugs are effective against HSV-1, HSV-2, and CMV, see, for instance, a recent meta-analysis of 30 aciclovir clinical trials in HSV infections (Leflore 2000¹³⁷⁴), a review on aciclovir recommended treatments in HSV infections (Kesson 1998¹³⁷⁵), reviews on valaciclovir effectiveness in HSV and CMV infections (Ormrod 2000¹³⁷⁶, Bell 1999¹³⁷⁷), and a review of famciclovir and penciclovir (Sacks 1999¹³⁷⁸).

(ii) Effect on Latent Viral DNA Load

The load of viral DNA during latent infection is directly correlated with the extent of viral replication during the preceding productive infection (Reddehase 1994¹³⁷⁹, Collins 1993¹³⁸⁰). Therefore, a decrease in viral replication should decrease the load of viral DNA during a subsequent latent infection. Consider the following observations.

A study (Steffens 1998¹³⁸¹) performed bone marrow transplantation (BMT) as a syngeneic BMT with female BALB/c (H-2^{d}) mice. Both donor and recipient mice were 8 weeks old. Two hours after BMT, the mice were infected subcutaneously in the left hind footpad with murine CMV. The mice were than divided into four groups. Three groups received therapy with increasing doses of CD8 T-cells. The forth groups served as controls. The results showed a significant dose-dependent decrease in extent and duration of virus replication in vital organs, such as lungs and adrenal glands, following treatment with CD8 T-cells (Steffens 1998, ibid, FIG. 2). Moreover, 12 months after BMT, the groups on CD8 T-cells therapy showed a decrease in the amount of viral DNA compared to controls. The viral DNA load in the lungs of mice given no immunotherapy was 5,000 viral genomes per 10⁶ lung cells. Viral load following treatment with 10⁵ and 10⁶ CD8 T-cells was 3,000 and 1,000 per 10⁶ lung cells, respectively. The study indicates that attenuated viral replication during the acute phase decreases viral DNA load during the subsequent latent phase.

The study (Steffens 1998, ibid) also measured the recurrence of viral infection following therapy. Five latently infected mice with no therapy, and five mice treated with 10⁷ CD8 T-cells were subjected to immunoablative γ-ray treatment of 6.5 Gy. Recurrence of viral infectivity was measured 14 days later in separate lobes of the lungs. The group receiving no therapy showed a high latent DNA load and recurrence of infectivity in all five mice in all five lobes of the lungs (with some variance). In contrast, the group receiving CD8 T-cells showed low viral load and recurrence of infectivity in only two mice and only in a single lobe in each mouse (Steffens 1998, ibid, FIG. 7). These observations indicate that a decrease in viral replication also decreases both latent viral DNA load and the probability viral disease.

Thackray and Field, in a series of studies, also tested the effect of preemptive therapy against viral infection. However, instead of CD8 T-cells, the studies administered famciclovir (FCV), valaciclovir (VACV), or human immunoglobulin (IgG), to mice infected via the ear pinna or the left side of the neck with HSV-1 or HSV-2 (Thackray 2000A¹³⁸², Thackray 2000B¹³⁸³, Thackray 2000C¹³⁸⁴, Field 2000¹³⁸⁵ Thackray 1998¹³⁸⁶). The results showed that 9-10 days of FCV treatment, early in infection, was effective in limiting the establishment of viral latency several months after treatment. Based on their observations, Field and Thackray (Field 2000, ibid) concluded: “Thus, the implication of our results is that even intensive antiviral therapy starting within a few hour of exposure is unlikely to completely abrogate latency. However, our results also show a significant reduction in the number of foci that are established and imply that there may also be a quantitative reduction in the latent genomes.”

Another study (LeBlanc 1999¹³⁸⁷) compared the effect of aciclovir (ACV) and immunoglobulin (IgG) preemptive therapy on mice infected with HSV-1 via scarified corneas. Both therapies were administered for 7 days starting on the first day post infection. The results showed that ACV treatment decreased the copy number of latent HSV-1 genomes on day 44-post infection relative to IgG (LeBlanc 1999, ibid, FIG. 5). Since untreated mice did not survive the infection, the study could not compare ACV treatment to no treatment. However, if we assume that IgG treatment did not change the copy number of latent viral genomes, we can conclude that ACV preemptive treatment decreases the load of latent viral DNA.

Ganciclovir is similar to aciclovir and penciclovir. Therefore, a reasonable conclusion from these studies is that preemptive treatment with ganciclovir will also decrease the load of latent viral DNA. Symbolically, [symbol]
[Ganciclovir]_{acute}→↓[N-boxᵥ]ₗₐₜₑₙₜ
Sequence of quantitative events 149: Predicted effect of ganciclovir on number of latent foreign N-boxes.

The symbol [Ganciclovir]_{acute} indicates treatment of ganciclovir during the acute phase, and the symbol [N-boxᵥ]ₗₐₜₑₙₜ indicates viral, N-box copy number during the latent phase.

(iii) Effect on Clinical Symptoms

(a) Atherosclerosis

Treatment with ganciclovir should decrease the rate of atherosclerosis progression. Accelerated coronary atherosclerosis can be observed in the donor heart following heart transplantation (TxCAD). Transplanting a heart from a CMV seropositive donor to a seronegative recipient increases the probability of a primary infection in the recipient (Bowden 1991¹³⁸⁸, Chou 1988¹³⁸⁹, Chou 1987¹³⁹⁰, Chou 1986¹³⁹¹, Grundy 1988¹³⁹², Grundy 1987¹³⁹³, Grundy 1986¹³⁹⁴). The Thackray and LeBlanc studies (see above) demonstrated that administration of aciclovir or penciclovir prophylaxis early in primary infection decreases the load of the subsequent latent viral DNA in the infected animals. Since microcompetition between viral and cellular DNA results in atherosclerosis, prophylactic administration of ganciclovir, a drug similar to aciclovir and penciclovir, early after heart transplantation, should decrease atherosclerosis. Symbolically, [symbol]
[Ganciclovir]prophylaxis→↓[N-boxᵥ]ₗₐₜₑₙₜ→↑[p30019 GABP·N-box_{G}]→ . . . →↓[Atherosclerosis]
Sequence of quantitative events 150: Predicted effect of ganciclover prophylaxis on susceptibility to atherosclerosis.

Consider the following observations. A study (Valantine 1999¹³⁹⁵) randomly treated 149 patients (131 men and 18 women, aged 48±13 years) with ganciclovir or placebo. Drug treatment started on the first postoperative day and was administered for 28 days. In 22% of patients, drug administration was delayed by up to 6 days due to acute-care problems. The study performed coronary angiography annually after heart transplantation, with mean follow-up time of 4.7±1.3 years. TxCAD was defined as the presence of any stenosis irrespective of severity because of the recognized underestimation of TxCAD by angiography. The actuarial incidence of TxCAD was determined from the annual angiograms and from autopsy data. CMV infection was determined in recipient and donor. The results showed a decrease in actuarial incidence of TxCAD at follow-up in patients treated with ganciclovir compared to patients treated with placebo (43±8% vs. 60±11%, p<0.1). Moreover, the protective effect of ganciclovir was more evident in the population of CMV seronegative recipients. In the CMV seronegative recipients, 4 (28%) of the 14 patients randomized to receive ganciclovir developed TxCAD compared to 9 (69%) of the 13 patients randomized to receive placebo. Base on these results, Valantine, et al., (1999, ibid) concluded: “prophylactic treatment with ganciclovir initiated immediately after heart transplantation decreases the incidence of TxCAD.”

Note: In a multivariate analysis, the study found that the variable “CMV illness” was not an independent predictor of TxCAD when the “lack of ganciclovir” and “donor age” variables were included in the analysis. It is possible that high correlation (multicollinearity) between “lack of ganciclovir” and “CMV illness” is responsible for the observed dependency. Such correlation was demonstrated in numerous studies. See, for instance, table 5 in Sia 2000¹³⁹⁶, which lists 10 clinical studies showing decreased CMV disease following early administration of ganciclovir prophylaxis in solid-organ transplantation compared to no treatment, administration of placebo, treatment with immunoglobulin, or treatment with acyclovir. From the correlation, it can be deduced that Valantine 1999 (ibid) also measured decreased CMV disease (the study is mute on the statistic). The key parameter that determines the overall and organ-specific risks of CMV disease is the copy number of latent viral genomes in various tissues (Reddehase 1994, ibid). Therefore, the decrease in CMV disease indicates a decrease in the copy number of latent viral genome, which explains the decrease in observed atherosclerosis.

The observations in Valantine 1999 (ibid) are consistent with the predicted effect of ganciclovir on atherosclerosis, a microcompetition disease.

(b) Zidovudine (AZT), Didanosine (ddI), Zalcitabine (ddC)

(i) Effect on Viral DNA Elongation

Didanosine (2′,3′-dideoxyinosine, ddI) is a synthetic purine nucleoside analogue used against HIV infection. After passive diffusion into the cell, the drug undergoes phosphorylation by cellular (rather than viral) enzymes to dideoxyadenosine-5′-triphosphate (ddATP), the active moiety. ddATP competes with the natural substrate for HIV-1 reverse transcriptase (deoxyadenosine 5′-triphosphate) and cellular DNA polymerase. Because ddATP lacks the 3′-hydroxyl group present in the naturally occurring nucleoside, incorporation into viral DNA leads to termination of DNA chain elongation and inhibition of viral DNA growth (see a recent review of ddI in Perry 1999¹³⁹⁷).

Zidovudine (retrovir, ZDV, AZT) and zalcitabine (ddC) are nucleosides similar to ddI.

(ii) Effect on Latent Viral DNA Load

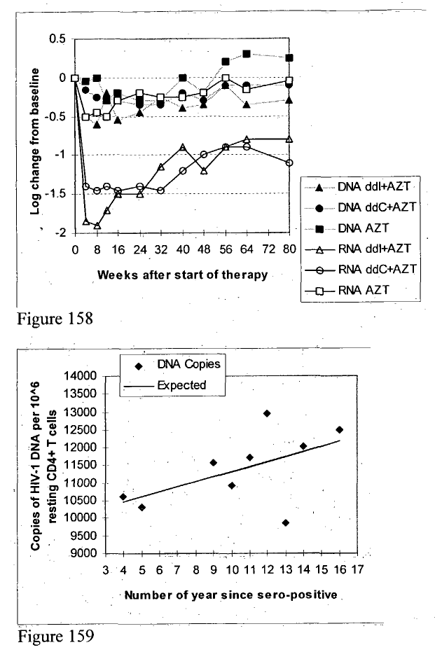

A study (Bruisten 1999¹³⁹⁸) measured the change in HIV-1 DNA and RNA load relative to baseline in 42 antiretroviral naive HIV-1 infected persons treated with AZT monotherapy, a combination of AZT+ddC, or a combination of AZT+ddI over a period of 80 weeks. FIG. 158 presents the results (Bruisten 1998, ibid, FIG. 1A).

At week 80, AZT treatment was associated with an increase, ddC+AZT with a small decrease and ddI+AZT with a larger decrease in viral DNA. To compare the results statistically, the mean log change from baseline over all time points was compared between ddI+AZT and ddC+AZT. The mean change was −0.3375 and −0.20458 for ddI+AZT and ddC+AZT, respectively (p=0.02). Note that, although not significant statistically (p=0.29), rank order of the ddI+AZT and ddC+AZT effect on RNA is reversed, that is, the mean effect of ddC+AZT on viral RNA was larger than ddI+AZT. Since the combination therapy of AZT and ddC is additive (Magnani 1997¹³⁹⁹), the ddC monotherapy effect on viral DNA was calculated as the ddC+AZT effect minus the AZT monotherapy effect. The calculated effect of ddC monotherapy on viral DNA was compared to the effect of AZT monotherapy. The mean log change from baseline over all time points was −0.15458 and −0.05 for ddC and AZT, respectively (p=0.09). The statistical analysis suggests that the ranking of ddI>ddC>AZT in terms of their effect on viral DNA, is significant. Moreover, the results suggest that at later time points, AZT tend to be associated with increased levels of viral DNA.

The above statistical analysis is different from the analysis reported in Bruisten 1998 (ibid). To test whether an “early” response occurred, the study averaged the values of weeks 4, 8, and 12, and for a “late” response, the study averaged the values of weeks 32, 40, and 48. The test showed that only the ddI+AZT treatment decreased HIV-1 viral DNA during the “early” and “late” periods. The p value for the “early” period compared to baseline was 0.002; the p value for the “late” period compare to baseline was 0.052. The same values for ddC+AZT during the “early” and “late” periods were 0.191 and 0.08, respectively. These values also indicate that ddI is more effective than ddC in decreasing viral DNA.

Another study (Pauza 1994¹⁴⁰⁰) measured total HIV-1 DNA in 51 infected patients by polymerase chain reaction (PCR) amplification of the viral LTR sequence. The assay detects linear, circular, and integrated HIV-1 DNA, including preintegration complexes that completed the first translocation step. Twenty patients were treated with AZT, 4 patients with ddI, and 7 patients with ddC. The measured LTR DNA levels were expressed on a scale of 1 to 5 (1 is lowest). Negative samples were labeled zero. The average ranking of viral DNA load for patients treated with ddI, ddC, and AZT, was 2.25, 2.71, and 2.74, respectively. The difference between ddC and AZT was small. However, the average CD4/μl count for ddC and AZT treated patients was 82 and 191.55, respectively (p<0.03 for the difference). Hence, the viral DNA load of the AZT group was most likely biased downward. Overall, the ranking of treatment effectiveness, measured in terms of decreased viral DNA load, is identical to the ranking reported in Bruisten 1998 (ibid) (see above).

Another study (Chun 1997¹⁴⁰¹) measured total HIV-1 DNA in 9 patients. Eight patients were on triple therapy including two nucleosides and one protease inhibitor. One patient received two nucleosides and two protease inhibitors. Six patients had undetectable plasma HIV RNA. The other three patients had 814, 2,800, and 6,518 copies/ml. The study also reports the year of seroconversion. A regression analysis with viral DNA level as dependent variable and number of years since seroconversion as independent variable produces the following results.
Viral DNA load 9,909+142×Years since seroconversion

The viral DNA load is measured in copies of HIV-1 DNA per 10⁶ resting CD4+ T-cells. The p values for the intercept and coefficient are 1.31 E-05 and 0.131481, respectively. Since the sample size is small, the p value for the coefficient are borderline significant. However, the direction of the regression line indicates that even in patients on triple and quadruple therapies, who show undetectable levels of plasma HIV RNA, viral DNA load increases with the number of years since seroconversion.

The difference between the expected and the observed number of viral DNA copies was calculated for each patient. The therapy of two patients included ddI and the average difference for these patients was −828 copies. The therapy of five patients included AZT and the average difference for these patients was +317 copies. These results suggest that ddI is associated with a decrease and AZT with an increase in the number of viral DNA copies in this group of patients.

To conclude: the observations in Bruisten 1998 (ibid), Pauza 1994 (ibid), and Chun 1997 (ibid) indicate that ddI is associated with a larger decrease in viral DNA load compared to ddC, and AZT is associated with an increase in viral DNA load. Note that the studies were performed under a variety of experimental conditions, with monotherapy, triple, and quadruple therapy with a protease inhibitor, and with detectable and undetectable RNA. Yet, the results are consistent in all studies.

(iii) Predictions and Observations: Effect on Clinical Symptoms

(a) Obesity

According to their effect on HIV DNA load, treatment with both ddI and ddC should decrease the rate of obesity progression. Moreover, treatment with ddI should be more effective than ddC in decreasing obesity. In contrast, treatment with AZT should increase the rate of obesity progression. Consider the following observations.

A study (Gervasoni 1999, ibid) observed 306 six HIV-infected women between December 1997 and February 1998. The women were treated with two or more antiretroviral drugs, 162 patients were treated with two nucleosides (double therapy), and 144 with three or more drugs including at least one protease inhibitor (PI) (triple therapy). Fat redistribution (FR) was confirmed by means of a physical examination and dual-energy X-ray absorptiometry (DEXA). FR was observed in 32 women (10.5%, 12 on double therapy, 20 on triple therapy). The body changes were reported to gradually emerge over a period of 12-72 weeks. A statistical analysis showed that a combination treatment that included ddI was significantly associated with absence of FR (p=0.019). A combination treatment that included ddC was also significantly associated with the absence of FR (p=0.049). The p values indicate that a ddI-including combination was more effective than a ddC-including therapy in preventing FR. Contrary to ddI and ddC, a combination therapy that included AZT was associated with a low risk of developing FR(OR 0.3).

The association between ddI-, ddC- and AZT-including therapeutic combinations with fat redistribution is consistent with their effect on viral DNA load. Consider the following table. Two arrows indicates larger change.

TABLE 30
Effect of ddI, ddC, and AZT treatment on HIV DNA and fat
redistribution.

| Treatment | Effect on HIV DNA | Effect on fat redistribution |
|---|---|---|
| ddI | ↓↓ | ↓↓ |
| ddC | ↓ | ↓ |
| AZT | ↑ | ↑ |

Another observation in Gervasoni 1999 (ibid) was the longer median total duration of antiretroviral drug treatment in women with FR compared to those without FR (1,187 versus 395 days). Only one of the 32 women with FR received antiretroviral drug therapy for less than 1,000 days. The risk of FR for women on antiretroviral drug therapy for more than 1,000 days was 10 times greater than in those who received shorter drug therapy (OR 10.8, p=0.0207). A statistical analysis of the results in Chun 1997 (ibid, see above) showed that viral DNA load increases with an increase in the number of years since seroconversion. Since the duration of antiretroviral drug treatment, most often, increases with the number of years since seroconversion, longer duration correlates with higher viral DNA load. Higher viral DNA load results in more intense microcompetition with the viral DNA, and therefore, increases fat redistribution.

The observations in Gervasoni 1999 (ibid) are consistent with the predicted effect of ddI, ddC, and AZT on obesity, a microcompetition disease.

(c) Garlic

(i) Effect on Viral Infectivity

Garlic shows antiviral activity; see, for instance, Guo 1993¹⁴⁰², and Weber 1992¹⁴⁰³.

(ii) Effect on Clinical Symptoms

See above.

(3) Immune Stimulating Agents

The balance between two forces, the virus drive to replicate, and the capacity of the immune system to control or clear the infection, determines the copy number of viral genome present in infected cells. A stable equilibrium between these two forces determines the copy number in persistent, latent infections. A major determinant of the immune system capacity to clear or control and infection is the efficiency of the Th 1 response. An increase in efficiency decreases viral copy number.

(a) Infection with non-GABP Viruses

Data obtained in animals indicate that neonatal immune responses are biased toward Th2. Consider a productive infection with a GABP virus during early life. The extent of viral replication during the productive phase determines the load of viral DNA during the subsequent latent phase (see discussion above). The lower the Th1 efficiency during the productive phase, the higher the copy number of viral genomes in the subsequent latent period. Infection with some viruses, such as measles, hepatitis A, and Mycobacterium tuberculosis, induces a strong polarized Th1-type response in early life. A concurrent infection with these viruses decreases replication of the GABP virus, which, in turn, decreases the copy number of the GABP virus genomes during the subsequent latent phase. The decreased copy number attenuates microcompetition between the viral DNA and cellular genes, which, in turn, decreases the probability and severity of microcompetition diseases. Let [Th1] denote Th1 efficiency, then,
↑[Th1]→↓[N-boxᵥ]ₗₐₜₑₙₜ→↑[p300·GABP·N-box_{G}]→ . . . →↓[Disease]
Sequence of quantitative events 151: Predicted effect of TH1 efficiency on susceptibility to a microcompetition disease.

BCG is a freeze-dried preparation made from a living culture of the Calmette-Guerin strain of mycobacterium Bovis. BCG was first developed in 1921 as a vaccine against tuberculosis but it had also been used as an immunotherapeutic treatment against carcinoma. Vaccination with BCG induces a Th1-type immune response in newborn and adult humans (Marchant 1999¹⁴⁰⁴). Moreover, BCG immunization prior to challenge with herpes simplex virus increased survival rate of newborn mice (Starr 1976¹⁴⁰⁵). Therefore, [symbol]
[BCG vaccination]→↑[Th1]→↓[N-boxᵥ]ₗₐₜₑₙₜ→↑[p300·GABP·N-box_{G}]→ . . . →↓[Disease]
Sequence of quantitative events 152: Predicted effect of BCG vaccination on susceptibility to a microcompetition disease.

Vaccination with BCG should decrease the rate of microcompetition disease progression. Consider the following observations.

A study (Aaby 2000¹⁴⁰⁶) compared the prevalence of atopy in children vaccinated with BCG in infancy and unvaccinated children. The study measured skin test reactivity to three allergens, Dermatophagoides pteronyssinus, D. farinae, and cockroach, in 400 children, aged 3-14 years, in an urban area of Bissau, the capital of Guinea-Bissau in West Africa. The results showed that 57 (21%) of the 271 vaccinated children were atopic (any reaction greater or equal 2 mm) compared with 21 (40%) of the 53 unvaccinated children (odds ratio=0.19 (95% CI 0.06-0.59) after controlling for potential confounding factors). When atopy was defined using a 3-mm criterion, earlier vaccination with BCD was associated with greater decrease in atopy, with the largest decrease observed in children vaccinated during the first week of life. Based on these results, Aaby, et al., (2000, ibid) concluded: “BCG vaccination given early in infancy may prevent the development of atopy in African children.”

The observations in Aaby 2000 (ibid) are consistent with the predicted effect of BCG vaccination on atopy, a microcompetition disease (see chapter on autoimmune disease, p 272).

Results of numerous studies suggest that an infection with measles, hepatitis A, or Mycobacterium tuberculosis in early life may prevent subsequent development of atopic diseases. In humans, immunomodulation during the first two years of life is most successful in producing long-lasting prevention effects (von Hertzen 2000¹⁴⁰⁷, see also von Mutius 2000¹⁴⁰⁸, von Hertzen 1999¹⁴⁰⁹). Encouraged by these effects, there are currently attempts to use BCG as a vaccine for asthma (see review Scanga 2000¹⁴¹⁰).

Another study (Shehadeh 1997¹⁴¹¹) evaluated the protective effect of repeated BCG vaccinations on type I diabetes in NOD mice. The results showed that 53% (17/32) of the control mice, 26% (8/31) of the single vaccine-treated mice (vaccination at age 35 days), 30% (7/23) of the single vaccine-treated mice (vaccination at age 90 days), and 0% (0/14) of the repeated BCG treated mice (vaccination at age 35 & 90 days), developed diabetes (p<0.05, repeated-vaccination group compared with controls and each of the single-vaccination groups). The mice repeatedly vaccinated with BCG did not develop the disease up to 250 days of age. The repeated BCG vaccination decreased the severity of insulitis at age 120 days as compared with controls and single BCG-vaccination groups. Based on the observations Shehadeh, et al., (1997 ibid) concluded: “Our report demonstrates that repeated BCG vaccination is safe and more effective than a single dose in preventing type I diabetes in NOD mice.”

The observations in Shehadeh 1997 (ibid) are consistent with the predicted effect of BCG vaccination on type I diabetes, a microcompetition disease (see chapter on autoimmune disease, p 272). On the relation between BCG immunization and type 1 diabetes, see also Qin 1997¹⁴¹², Harada 1990¹⁴¹³ and a recent review Hiltunen 1999¹⁴¹⁴.

Another study (Martins 1999¹⁴¹⁵) showed that an infection of NOD mice with Mycobacterium avium, before the mice show overt diabetes, results in permanent protection of the animals from diabetes. The protective effect was associated with increased numbers of CD4+ T-cells and B220+B cells. The study also showed that the protection was associated with changes in the expression of Fas (CD95) and FasL by immune cells, and alterations in cytotoxic activity, IFNγ and IL-4 production and activation of T-cells of infected animals. Based on these results, Martins and Aguas (1999, ibid) concluded: the “data indicate that protection of NOD mice from diabetes is a Th I-type response that is mediated by up-regulation of the Fas-FasL pathway and involves an increase in the cytotoxicity of T-cells.” See also Bras 1996¹⁴¹⁶.

(b) Breast-Feeding

Several studies showed an increase in efficiency of the Th1 immune response in breast-fed children. Pabst 1997A¹⁴¹⁷ compared the blast transformation and cytokine production by lymphocytes, and T-cells, in 59 formula-fed, and 64 breast-fed 12-month-old children, before and after measles-mumps-rubella vaccination (MMR). The results showed decreased levels of blast transformation without antigen (p<0.001), with tetanus toxoid (p<0.02), or Candida (p<0.04), and lower IFNγ production (p<0.03), in breast-fed children before vaccination. Fourteen days following vaccination with a live virus, breast-fed children showed an increase in production of IFNγ (p<0.02), and an increase in percentages of CD56+(p<0.022) and CD8+ cells (p<0.004). Formula-fed children showed no significant response to vaccination. Based on these results, Pabst, et al., (1997, ibid) concluded: “these findings are consistent with a Th1 type response by breast fed children, not evident in formula-fed children. Feeding mode has an important long-term immunomodulating effect on infants beyond weaning.” See also the review Pabst 1997B¹⁴¹⁸.

Another study showed immunophenotypic differences between breast-fed and formula-fed infants consistent with accelerated development of immune system in breast-fed infants (Hawkes 1999¹⁴¹⁹).

Since breast-feeding increases the efficiency of the Th1 immune response, it should decrease the rate of microcompetition disease progression. Symbolically, [symbol]
[Breast-feeding]→↑[Th1]→↓[N-boxᵥ]ₗₐₜₑₙₜ→↑[p30019 GABP·N-box_{G}]→ . . . →↓[Disease]
Sequence of quantitative events 153: Predicted effect of breast-feeding on susceptibility to a microprocompetition disease.

Consider the following observations. A study (Pettitt 1997¹⁴²⁰) examined the relation between breast-feeding and type II diabetes (non-insulin-dependent diabetes, or NIDDM) in 720 Pima Indians, age 10-39 years, 144 exclusively breast-fed, 325 exclusively bottle-fed, and 251 mixed-fed. The exclusively breast-fed participants, or mixed-fed participants, showed lower age-adjusted and sex-adjusted mean relative body weight compared to bottle-fed participants (140%, 139%, and 146%, p=0.019). The exclusively breast-fed participants also showed a significant lower rate of NIDDM compared to the exclusively bottle-fed participants in all age groups (odds ratio=0.41 (95% CI 0.18-0.93), adjusted for age, sex, birth date, parental diabetes, and birth weight). Based on these observations, Pettitt, et al., (1997, ibid) concluded: “exclusive breast-feeding for the first 2 months of life is associated with a significantly lower rate of NIDDM in Pima Indians.”

Several other studies reported a similar inverse relation between breast-feeding and diabetes (Virtanen 1992¹⁴²¹, Virtanen 1991¹⁴²², Borch-Johnsen 1984¹⁴²³).

Another study (von Kries 2000¹⁴²⁴) measured the effect of breast-feeding on overweight and obesity in 9,206 children at school entry (age 5 or 6) in a cross sectional study in Bavaria in 1997. Overweight was defined as BMI>90th percentile (calculated using data on 134,577 German children seen at the 1997 school entry health examination in Bavaria), and obesity as BMI>97th percentile. Out of the 9,206 children in the study, 56% had been breast-fed for any length of time. The results showed a decrease in the upper tail of the BMI distribution for breast-fed children compared to non breast-fed children. The median of the two BMI distributions was similar. Prevalence of obesity was 2.8% and 4.5% in breast-fed children and non breast-fed children, respectively. Duration of breast-feeding showed a dose response effect on prevalence of obesity. Exclusive breast-feeding for up to 2, 3 to 5, 6 to 12, and more than 12 months, was associated with 3.8%, 2.3%, 1.7%, and 0.8% prevalence of obesity, respectively. Breast-feeding and overweight showed similar relations. The odds ratios of breast-feeding, for any length of time, adjusted for differences in social class or lifestyle, was 0.71 (95% CI 0.56-0.90) for obesity, and 0.77 (95% CI 0.66-0.88) for overweight. The odds ratios suggest that differences in social class or lifestyle cannot explain the effect of breast-feeding on overweight and obesity. Moreover, the study notes that, in a similar study (von Kries 1999¹⁴²⁵), maternal overweight also could not explain the effect of breastfeeding on overweight and obesity. Based on these observations, von Kries, et al., (2000, ibid) concluded: “The reduction in the risk for overweight and obesity is therefore more likely to be related to the properties of human milk than to factors associated with breast-feeding.

Several other studies reported a similar inverse relation between breast-feeding and obesity (Bergmann 2003¹⁴²⁶, Armstrong 2002¹⁴²⁷, Toschke 2002¹⁴²⁸, Liese 2001¹⁴²⁹, Hediger 2001¹⁴³⁰, Gillman 2001¹⁴³¹, see also editorials Gillman 2002¹⁴³², Dietz 2001¹⁴³³).

The observations in these studies are consistent with the predicted effect of breast-feeding on obesity and type II diabetes, two microcompetition diseases.

17. Concluding Remarks

The examples section of the specifications presents a theory that identifies the origin of many chronic diseases. According to Albert Einstein:
“A theory is more impressive the greater the simplicity of its premises, the more different kinds of things it relates, and the more extended its area of applicability” (Einstein 1951, ibid, p. 33).

The theory presented in the specifications is based on one basic premise: microcompetition with foreign DNA causes chronic disease. The derived conclusions (the subsequent events in the different sequences of quantitative events) relate numerous seemingly unrelated observations reported in studies with animals, humans, in vitro, in vivo, on a molecular level, cellular level, clinical level, on atherosclerosis, cancer, obesity, osteoarthritis, type II diabetes, alopecia, type I diabetes, multiple sclerosis, asthma, lupus, thyroiditis, inflammatory bowel disease, rheumatoid arthritis, psoriasis, atopic dermatitis, graft versus host disease, and other chronic diseases. To use Einstein's criteria, a theory based on a single premise, which relates so many seemingly unrelated observations, from such a diversity of topics, is a good theory.

Using this theory about the cause of chronic disease, new methods were developed to evaluate the effectiveness of a compound for use in modulating the progression of chronic disease, to determine whether a subject has a chronic disease, or has an increased risk of developing clinical symptoms associated with such disease, and to treat chronic disease.

### TABLE OF CITED REFERENCES

¹ Harmon, Richard. http://www.siliconinvestor.com/stocktalk/msg.gsp?msgid=18211591.
² http://www.bartleby.com/61.
³ http://www.merck.com/!!EIB2jbottEIB2jbo/careers/mrl/basic.html.
⁴ http://www.glaxowellcom.co.uk/science/targint.html.
⁵ Werth, Barry. The Billion Dollar Molecule, One Company's Quest for the Perfect Drug. New York, Simon and Schuster. 1995; 360-361.
⁶ Werth, Barry. The Billion Dollar Molecule, One Company's Quest for the Perfect Drug. New York, Simon and Schuster. 1995; 234.
⁷ Capell, Kerry. http://www.businessweek.com:/print/premium/content/03_{—}21/b3834028_mz044.htm?gb.
⁸ Molecular target drug discovery for cancer: exploratory grants, Natiaonal Cancer Institute, Feb. 16, 2000, http://grants.nih.gov/grants/guide/pa-files/PAR-00-060.html
⁹ Gonelli A, Boccia S, Boni M, Pozzoli A, Rizzo C, Querzoli P, Cassai E, Di Luca D. Human herpesvirus 7 is latent in gastric mucosa. J Med Virol. 2001 April; 63(4):277-83.
¹⁰ Smith R L, Morroni J, Wilcox C L. Lack of effect of treatment with penciclovir or acyclovir on the establishment of latent HSV-1 in primary sensory neurons in culture. Antiviral Res. 2001 October; 52(1):19-24.
¹¹ Young L S, Dawson C W, Eliopoulos A G. The expression and function of Epstein-Barr virus encoded latent genes. Mol Pathol. 2000 October; 53(5):238-47.
¹² Vo N, Goodman R H. CREB-binding protein and p300 in transcriptional regulation. J Biol. Chem. 2001 Apr. 27; 276(17):13505-8.
¹³ Blobel G A. CREB-binding protein and p300: molecular integrators of hematopoietic transcription. Blood. 2000 Feb. 1; 95(3):745-55.
¹⁴ Goodman R H, Smolik S. CBP/p300 in cell growth, transformation, and development. Genes Dev. 2000 Jul. 1; 14(13):1553-77.
¹⁵ Hottiger M O, Nabel G J. Viral replication and the coactivators p300 and CBP. Trends Microbiol. 2000 December; 8(12):560-5.
¹⁶ Giordano A, Avantaggiati M L. p300 and CBP: partners for life and death. J Cell Physiol. 1999 November; 181(2):218-30.
¹⁷ Eckner R. p300 and CBP as transcriptional regulators and targets of oncogenic events. Biol. Chem. 1996 November; 377(11):685-8.
¹⁸ Kitabayashi I, Yokoyama A, Shimizu K, Ohki M. Interaction and functional cooperation of the leukemia-associated factors AML1 and p300 in myeloid cell differentiation. EMBO J. 1998 Jun. 1; 17(11):2994-3004.
¹⁹ Facchinetti V, Loffarelli L, Schreek S, Oelgeschlager M, Luscher B, Introna M, Go. Regulatory domains of the A-Myb transcription factor and its interaction with the CBP/p300 adaptor molecules. Biochem J. 1997 Jun. 15; 324 (Pt 3):729-36.
²⁰ Duyndam M C, van Dam H, Smits P H, Verlaan M, van der Eb A J, Zantema A. The N-terminal transactivation domain of ATF2 is a target for the co-operative activation of the c-jun promoter by p300 and 12S E1A. Oncogene. 1999 Apr. 8; 18(14):2311-21.
²¹ Yukawa K, Tanaka T, Tsuji S, Akira S. Regulation of transcription factor C/ATF by the cAMP signal activation in hippocampal neurons, and molecular interaction of C/ATF with signal integrator CBP/p300. Brain Res Mol Brain Res. 1999 May 21; 69(1):124-34.
²² Mink S, Haenig B, Klempnauer K H. Interaction and functional collaboration of p300 and C/EBPbeta. Mol Cell Biol. 1997 November; 17(11):6609-17.
²³ Yanagi Y, Masuhiro Y, Mori M, Yanagisawa J, Kato S. p300/CBP acts as a coactivator of the cone-rod homeobox transcription factor. Biochem Biophys Res Commun. 2000 Mar. 16; 269(2):410-4.
²⁴ Lamprecht C, Mueller C R. D-site binding protein transactivation requires the proline- and acid-rich domain and involves the coactivator p300. J Biol. Chem. 1999 Jun. 18; 274(25):17643-8.
²⁵ Marzio G, Wagener C, Gutierrez M I, Cartwright P, Helin K, Giacca M. E2F family members are differentially regulated by reversible acetylation. J Biol Chem 2000 Apr. 14; 275(15):10887-92.
²⁶ Silverman E S, Du J, Williams A J, Wadgaonkar R, Drazen J M, Collins T. cAMP-response-element-binding-protein-binding protein (CBP) and p300 are transcriptional co-activators of early growth response factor-1 (Egr-1). Biochem J. 1998 Nov. 15; 336 (Pt 1): 183-9.
²⁷ Kim M Y, Hsiao S J, Kraus W L. A role for coactivators and histone acetylation in estrogen receptor alpha-mediated transcription initiation. EMBO J. 2001 Nov. 1; 20(21):6084-94.
²⁸ Wang C, Fu M, Angeletti R H, Siconolfi-Baez L, Reutens A T, Albanese C, Lisanti M P, Katzenellenbogen B S, Kato S, Hopp T, Fuqua S A, Lopez G N, Kushner P J, Pestell R G. Direct acetylation of the estrogen receptor alpha hinge region by p300 regulates transactivation and hormone sensitivity. J Biol. Chem. 2001 May 25; 276(21): 18375-83.
²⁹ Speir E, Yu Z X, Takeda K, Ferrans V J, Cannon R O 3rd. Competition for p300 regulates transcription by estrogen receptors and nuclear factor-kappaB in human coronary smooth muscle cells. Circ Res. 2000 Nov. 24; 87(11):1006-11.
³⁰ Kobayashi Y, Kitamoto T, Masuhiro Y, Watanabe M, Kase T, Metzger D, Yanagisawa J, Kato S. p300 mediates functional synergism between AF-1 and AF-2 of estrogen receptor alpha and beta by interacting directly with the N-terminal A/B domains. J Biol Chem 2000 May 26; 275(21): 15645-51.
³¹ Papoutsopoulou S, Janknecht R. Phosphorylation of ETS transcription factor ER81 in a complex with its coactivators CREB-binding protein and p300. Mol Cell Biol. 2000 October; 20(19):7300-10.
³² Jayaraman G, Srinivas R, Duggan C, Ferreira E, Swaminathan S, Somasundaram K, Williams J, Hauser C, Kurkinen M, Dhar R, Weitzman S, Buttice G, Thimmapaya B. p300/cAMP-responsive element-binding protein interactions with ets-1 and ets-2 in the transcriptional activation of the human stromelysin promoter. J Biol. Chem. 1999 Jun. 11; 274(24):17342-52.
³³ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.
³⁴ Bhattacharya S, Michels C L, Leung M K, Arany Z P, Kung A L, Livingston D M. Functional role of p35srj, a novel p300/CBP binding protein, during transactivation by HIF-1. Genes Dev. 1999 Jan. 1; 13(1):64-75.
³⁵ Kallio P J, Okamoto K, O'Brien S, Carrero P, Makino Y, Tanaka H, Poellinger L. Signal transduction in hypoxic cells: inducible nuclear translocation and recruitment of the CBP/p300 coactivator by the hypoxia-inducible factor-1alpha. EMBO J. 1998 Nov. 16; 17(22):6573-86.
³⁶ Ema M, Hirota K, Mimura J, Abe H, Yodoi J, Sogawa K, Poellinger L, Fujii-Kuriyama Y. Molecular mechanisms of transcription activation by HLF and HIF1alpha in response to hypoxia: their stabilization and redox signal-induced interaction with CBP/p300. EMBO J. 1999 Apr. 1; 18(7): 1905-14.
³⁷ Soutoglou E, Papafotiou G, Katrakili N, Talianidis I. Transcriptional activation by hepatocyte nuclear factor-1 requires synergism between multiple coactivator proteins. J Biol. Chem. 2000 Apr. 28; 275(17):12515-20.
³⁸ Yoneyama M, Suhara W, Fukuhara Y, Fukuda M, Nishida E, Fujita T. Direct triggering of the type I interferon system by virus infection: activation of a transcription factor complex containing IRF-3 and CBP/p300. EMBO J. 1998 Feb. 16; 17(4):1087-95.
³⁹ Sato S, Roberts K, Gambino G, Cook A, Kouzarides T, Goding C R. CBP/p300 as a co-factor for the Microphthalmia transcription factor. Oncogene. 1997 Jun. 26; 14(25):3083-92.
⁴⁰ Sartorelli V, Huang J, Hamamori Y, Kedes L. Molecular mechanisms of myogenic coactivation by p300: direct interaction with the activation domain of MyoD and with the MADS box of MEF2C. Mol Cell Biol. 1997 February; 17(2):1010-26.
⁴¹ Garcia-Rodriguez C, Rao A. Nuclear factor of activated T cells (NFAT)-dependent transactivation regulated by the coactivators p300/CREB-binding protein (CBP). J Exp Med. 1998 Jun. 15; 187(12):2031-6.
⁴² Sisk T J, Gourley T, Roys S, Chang C H. MHC class II transactivator inhibits IL-4 gene transcription by competing with NF-AT to bind the coactivator CREB binding protein (CBP)/p300. J. Immunol. 2000 Sep. 1; 165(5):2511-7.
⁴³ Li Q, Herrler M, Landsberger N, Kaludov N, Ogryzko V V, Nakatani Y, Wolffe A P. Xenopus NF-Y pre-sets chromatin to potentiate p300 and acetylation-responsive transcription from the Xenopus hsp70 promoter in vivo. EMBO J. 1998 Nov. 2; 17(21):6300-15.
⁴⁴ Faniello M C, Bevilacqua M A, Condorelli G, de Crombrugghe B, Maity S N, Avvedimento V E, Cimino F, Costanzo F. The B subunit of the CAAT-binding factor NFY binds the central segment of the Co-activator p300. J Biol. Chem. 1999 Mar. 19; 274(12):7623-6.
⁴⁵ Gerritsen M E, Williams A J, Neish A S, Moore S, Shi Y, Collins T. CREB-binding protein/p300 are transcriptional coactivators of p65. Proc Natl Acad Sci USA. 1997 Apr. 1; 94(7):2927-32.
⁴⁶ Iannone M A, Consler T G, Pearce K H, Stimmel J B, Parks D J, Gray J G. Multiplexed molecular interactions of nuclear receptors using fluorescent microspheres. Cytometry 2001 Aug. 1; 44(4):326-37.
⁴⁷ Kodera Y, Takeyama K, Murayama A, Suzawa M, Masuhiro Y. Kato S. Ligand type-specific interactions of peroxisome proliferator-activated receptor gamma with transcriptional coactivators. J Biol Chem 2000 Oct. 27; 275(43):33201-4.
⁴⁸ Han B, Liu N, Yang X, Sun H B, Yang Y C. MRG1 expression in fibroblasts is regulated by Sp1/Sp3 and an Ets transcription factor. J Biol. Chem. 2001 Mar. 16; 276(11):7937-42.
⁴⁹ Avantaggiati M L, Ogryzko V, Gardner K, Giordano A, Levine A S, Kelly K. Recruitment of p300/CBP in p53-dependent signal pathways. Cell. 1997 Jun. 27; 89(7):1175-84.
⁵⁰ Van Orden K, Giebler H A, Lemasson I, Gonzales M, Nyborg J K. Binding of p53 to the KIX domain of CREB binding protein. A potential link to human T-cell leukemia virus, type I-associated leukemogenesis. J Biol. Chem. 1999 Sep. 10; 274(37):26321-8.
⁵¹ Yang W, Hong Y H, Shen X Q, Frankowski C, Camp H S, Leff T. Regulation of transcription by AMP-activated protein kinase: phosphorylation of p300 blocks its interaction with nuclear receptors. J Biol Chem 2001 Oct. 19; 276(42):38341-4.
⁵² Janknecht R, Wells N J, Hunter T. TGF-beta-stimulated cooperation of smad proteins with the coactivators CBP/p300. Genes Dev. 1998 Jul. 15; 12(14):2114-9.
⁵³ Feng X H, Zhang Y, Wu R Y, Derynck R. The tumor suppressor Smad4/DPC4 and transcriptional adaptor CBP/p300 are coactivators for smad3 in TGF-beta-induced transcriptional activation. Genes Dev. 1998 Jul. 15; 12(14):2153-63.
⁵⁴ de Caestecker M P, Yahata T, Wang D, Parks W T, Huang S, Hill C S, Shioda T, Roberts A B, Lechleider R J. The Smad4 activation domain (SAD) is a proline-rich, p300-dependent transcriptional activation domain. J Biol Chem 2000 Jan. 21; 275(3):2115-22.
⁵⁵ Pearson K L, Hunter T, Janknecht R. Activation of Smad1-mediated transcription by p300/CBP. Biochim Biophys Acta 1999 Dec. 23; 1489(2-3):354-64.
⁵⁶ Pouponnot C, Jayaraman L, Massague J. Physical and functional interaction of SMADs and p300/CBP. J Biol. Chem. 1998 Sep. 4; 273(36):22865-8.
⁵⁷ Oliner J D, Andresen J M, Hansen S K, Zhou S, Tjian R. SREBP transcriptional activity is mediated through an interaction with the CREB-binding protein. Genes Dev 1996 Nov. 15; 10(22):2903-11.
⁵⁸ Paulson M, Pisharody S, Pan L, Guadagno S, Mui A L, Levy D E. Stat protein transactivation domains recruit p300/CBP through widely divergent sequences. J Biol. Chem. 1999 Sep. 3; 274(36):25343-9.
⁵⁹ Zhang J J, Vinkemeier U, Gu W, Chakravarti D, Horvath C M, Darnell J E Jr. Two contact regions between Stat1 and CBP/p300 in interferon gamma signaling. Proc Natl Acad Sci USA. 1996 Dec. 24; 93(26):15092-6.
⁶⁰ Bhattacharya S, Eckner R, Grossman S, Oldread E, Arany Z, D'Andrea A, Livingston D M. Cooperation of Stat2 and p300/CBP in signalling induced by interferon-alpha. Nature. 1996 Sep. 26; 383(6598):344-7.
⁶¹ Pfitzner E, Jahne R, Wissler M, Stoecklin E, Groner B. p300/CREB-binding protein enhances the prolactin-mediated transcriptional induction through direct interaction with the transactivation domain of Stat5, but does not participate in the Stat5-mediated suppression of the glucocorticoid response. Mol Endocrinol. 1998 October; 12(10):1582-93.
⁶² Gingras S, Simard J, Groner B, Pfitzner E. p300/CBP is required for transcriptional induction by interleukin-4 and interacts with Stat6. Nucleic Acids Res. 1999 Jul. 1; 27(13):2722-9.
⁶³ Hamamori Y, Sartorelli V, Ogryzko V, Puri P L, Wu H Y, Wang J Y, Nakatani Y, Kedes L. Regulation of histone acetyltransferases p300 and PCAF by the bHLH protein twist and adenoviral oncoprotein E1A. Cell 1999 Feb. 5; 96(3):405-13.
⁶⁴ Manning E T, Ikehara T, Ito T, Kadonaga J T, Kraus W L. p300 forms a stable, template-committed complex with chromatin: role for the bromodomain. Mol Cell Biol. 2001 June; 21(12):3876-87.
⁶⁵Kraus W L, Manning E T, Kadonaga J T. Biochemical analysis of distinct activation functions in p300 that enhance transcription initiation with chromatin templates. Mol Cell Biol. 1999 December; 19(12):8123-35.
⁶⁶ Kraus W L, Kadonaga J T. p300 and estrogen receptor cooperatively activate transcription via differential enhancement of initiation and reinitiation. Genes Dev. 1998 Feb. 1; 12(3):331-42.
⁶⁷ Rosmarin A G, Luo M, Caprio D G, Shang J, Simkevich C P. Sp1 Cooperates with the ets Transcription Factor, GABP, to Activate the CD18 (62 Leukocyte Integrin) Promoter. Journal of Biological Chemistry 1998 273(21): 13097-13103.
⁶⁸ Avots A, Hoffmeyer A, Flory E, Cimanis A, Rapp U R, Serfling E. GABP factors bind to a distal interleukin 2 (IL-2) enhancer and contribute to c-Raf-mediated increase in IL-2 induction. Molecular and Cellular Biology 1997 17(8):4381-4389.
⁶⁹ Lin J X, Bhat N K, John S, Queale W S, Leonard W J. Characterization of the human interleukin-2 receptor beta-chain gene promoter: regulation of promoter activity by ets gene products. Mol Cell Biol. 1993 October; 13(10):6201-10.
⁷⁰ Markiewicz S, Bosselut R, Le Deist F, de Villartay J P, Hivroz C, Ghysdael J, Fischer A, de Saint Basile G. Tissue-specific activity of the gammac chain gene promoter depends upon an Ets binding site and is regulated by GA-binding protein. J Biol. Chem. 1996 Jun. 21; 271(25):14849-55.
⁷¹ Smith M F Jr, Carl V S, Lodie T, Fenton M J. Secretory interleukin-1 receptor antagonist gene expression requires both a PU.1 and a novel composite NF-kappaB/PU.1/GA-binding protein binding site. J Biol. Chem. 1998 Sep. 11; 273(37):24272-9.
⁷² Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. 1997 Aug. 1; 57(15):3145-8.
⁷³ Kamura T, Handa H, Hamasaki N, Kitajima S. Characterization of the human thrombopoietin gene promoter. A possible role of an Ets transcription factor, E4TF1/GABP. J Biol. Chem. 1997 Apr. 25; 272(17):11361-8.
⁷⁴ Wang K, Bohren K M, Gabbay K H. Characterization of the human aldose reductase gene promoter. J Biol. Chem. 1993 Jul. 25; 268(21):16052-8.
⁷⁵ Nuchprayoon I, Shang J, Simkevich C P, Luo M, Rosmarin A G, Friedman A D. An enhancer located between the neutrophil elastase and proteinase 3 promoters is activated by Sp1 and an Ets factor. J Biol. Chem. 1999 Jan. 8; 274(2): 1085-91.
⁷⁶ Nuchprayoon I, Simkevich C P, Luo M, Friedman A D, Rosmarin A G. GABP cooperates with c-Myb and C/EBP to activate the neutrophil elastase promoter. Blood. 1997 Jun. 15; 89(12):4546-54.
⁷⁷ Sadasivan E, Cedeno M M, Rothenberg S P. Characterization of the gene encoding a folate-binding protein expressed in human placenta. Identification of promoter activity in a G-rich SP1 site linked with the tandemly repeated GGAAG motif for the ets encoded GA-binding protein. J Biol. Chem. 1994 Feb. 18; 269(7):4725-35.
⁷⁸ Basu A, Park K, Atchison M L, Carter R S, Avadhani N G. Identification of a transcriptional initiator element in the cytochrome c oxidase subunit Vb promoter which binds to transcription factors NF-E1 (YY-1, delta) and Sp 1. J Biol. Chem. 1993 Feb. 25; 268(6):4188-96.
⁷⁹ Sucharov C, Basu A, Carter R S, Avadhani N G. A novel transcriptional initiator activity of the GABP factor binding ets sequence repeat from the murine cytochrome c oxidase Vb gene. Gene Expr. 1995; 5(2):93-111.
⁸⁰ Carter R S, Avadhani N G. Cooperative binding of GA-binding protein transcription factors to duplicated transcription initiation region repeats of the cytochrome c oxidase subunit IV gene. J Biol. Chem. 1994 Feb. 11; 269(6):4381-7.
⁸¹ Carter R S, Bhat N K, Basu A, Avadhani N G. The basal promoter elements of murine cytochrome c oxidase subunit IV gene consist of tandemly duplicated ets motifs that bind to GABP-related transcription factors. J Biol. Chem. 1992 Nov. 15; 267(32):23418-26.
⁸² Virbasius J V, Scarpulla R C. Activation of the human mitochondrial transcription factor A gene by nuclear respiratory factors: a potential regulatory link between nuclear and mitochondrial gene expression in organelle biogenesis. Proc Natl Acad Sci USA. 1994 Feb. 15; 91(4):1309-13.
⁸³ Villena J A, Vinas O, Mampel T, Iglesias R, Giralt M, Villarroya F. Regulation of mitochondrial biogenesis in brown adipose tissue: nuclear respiratory factor-2/GA-binding protein is responsible for the transcriptional regulation of the gene for the mitochondrial ATP synthase beta subunit. Biochem J. 1998 Apr. 1; 331 (Pt 1):121-7.
⁸⁴ Ouyang L, Jacob K K, Stanley F M. GABP mediates insulin-increased prolactin gene transcription. J Biol. Chem. 1996 May 3; 271(18):10425-8.
⁸⁵ Hoare S, Copland J A, Wood T G, Jeng Y J, Izban M G, Soloff M S. Identification of a GABP alpha/beta binding site involved in the induction of oxytocin receptor gene expression in human breast cells, potentiation by c-Fos/c-Jun. Endocrinology. 1999 May; 140(5):2268-79.
⁸⁶ Mantovani R. A survey of 178 NF-Y binding CCAAT boxes. Nucleic Acids Res. 1998 Mar. 1; 26(5): 1135-43.
⁸⁷ Espinos E, Le Van Thai A, Pomies C, Weber M J. Cooperation between phosphorylation and acetylation processes in transcriptional control. Mol Cell Biol 1999 May; 19(5):3474-84.
⁸⁸ Shiraishi M, Hirasawa N, Kobayashi Y, Oikawa S, Murakami A, Ohuchi K. Participation of mitogen-activated protein kinase in thapsigargin- and TPA-induced histamine production in murine macrophage RAW 264.7 cells. Br J Pharmacol 2000 February; 129(3):515-24.
⁸⁹ Herrera R, Hubbell S, Decker S, Petruzzelli L. A role for the MEK/MAPK pathway in PMA-induced cell cycle arrest: modulation of megakaryocytic differentiation of K562 cells. Exp Cell Res 1998 Feb. 1; 238(2):407-14.
⁹⁰ Stadheim T A, Kucera G L. Extracellular signal-regulated kinase (ERK) activity is required for TPA-mediated inhibition of drug-induced apoptosis. Biochem Biophys Res Commun 1998 Apr. 7; 245(1):266-71.
⁹¹ Yen A, Roberson M S, Varvayanis S. Retinoic acid selectively activates the ERK2 but not JNK/SAPK or p38 MAP kinases when inducing myeloid differentiation. In vitro Cell Dev Biol Anim. 1999 October; 35(9):527-32.
⁹² Liu M K, Brownsey R W, Reiner N E. F interferon induces rapid and coordinate activation of mitogen-activated protein kinase (extracellular signal-regulated kinase) and calcium-independent protein kinase C in human monocytes. Infect Immun, July 1994, 2722-2731, Vol 62, No. 7.
⁹³ Nishiya T, Uehara T, Edamatsu H, Kaziro Y, Itoh H, Nomura Y. Activation of Stat1 and subsequent transcription of inducible nitric oxide synthase gene in C6 glioma cells is independent of interferon-γ-induced MAPK activation that is mediated by p21ras. FEBS Lett 1997 May 12; 408(1):33-8.
⁹⁴ Lessor T, Yoo J Y, Davis M, Hamburger A W. Regulation of heregulin beta1-induced differentiation in a human breast carcinoma cell line by the extracellular-regulated kinase (ERK) pathway. J Cell Biochem 1998 Sep. 15; 70(4):587-95.
⁹⁵ Marte B M, Graus-Porta D, Jeschke M, Fabbro D, Hynes N E, Taverna D. NDF/heregulin activates MAP kinase and p70/p85 S6 kinase during proliferation or differentiation of mammary epithelial cells. Oncogene 1995 Jan. 5; 10(1):167-75.
⁹⁶ Sepp-Lorenzino L, Eberhard I, Ma Z, Cho C, Serve H, Liu F. Rosen N, Lupu R. Signal transduction pathways induced by heregulin in MDA-MB-453 breast cancer cells. Oncogene 1996 Apr. 18; 12(8):1679-87.
⁹⁷ Fiddes R J, Janes P W, Sivertsen S P, Sutherland R L, Musgrove E A, Daly R J. Inhibition of the MAP kinase cascade blocks heregulin-induced cell cycle progression in T-47D human breast cancer cells. Oncogene 1998 May 28; 16(21):2803-13.
⁹⁸ Park J A, Koh J Y. Induction of an immediate early gene egr-1 by zinc through extracellular signal-regulated kinase activation in cortical culture: its role in zinc-induced neuronal death. J. Neurochem. 1999 August; 73(2):450-6.
⁹⁹ Kiss Z, Crilly K S, Tomono M. Bombesin and zinc enhance the synergistic mitogenic effects of insulin and phosphocholine by a MAP, kinase-dependent mechanism in Swiss 3T3 cells. FEBS Lett. 1997 Sep. 22; 415(1):71-4.
¹⁰⁰ Wu W, Graves L M, Jaspers I, Devlin R B, Reed W, Samet J M. Activation of the EGF receptor signaling pathway in human airway epithelial cells exposed to metals. Am J. Physiol. 1999 November; 277(5 Pt 1):L924-31.
¹⁰¹ Samet J M, Graves L M, Quay J, Dailey L A, Devlin R B, Ghio A J, Wu W, Bromberg P A, Reed W. Activation of MAPKs in human bronchial epithelial cells exposed to metals. Am J. Physiol. 1998 September; 275(3 Pt 1):L551-8.
¹⁰² Migliaccio A, Di Domenico M, Castoria G, de Falco A, Bontempo P, Nola E, Auricchio F. Tyrosine kinase/p21ras/MAP-kinase pathway activation by estradiol-receptor complex in MCF-7 cells. EMBO J. 1996 Mar. 15; 15(6):1292-300.
¹⁰³ Ruzycky A L. Effects of 17 beta-estradiol and progesterone on mitogen-activated protein kinase expression and activity in rat uterine smooth muscle. Eur J. Pharmacol. 1996 Apr. 11; 300(3):247-54.
¹⁰⁴ Nuedling S, Kahlert S, Loebbert K, Meyer R, Vetter H, Grohe C. Differential effects of 17beta-estradiol on mitogen-activated protein kinase pathways in rat cardiomyocytes. FEBS Lett. 1999 Jul. 9; 454(3):271-6.
¹⁰⁵ Laporte J D, Moore P E, Abraham J H, Maksym G N, Fabry B, Panettieri R A Jr, Shore S A. Role of ERK MAP kinases in responses of cultured human airway smooth, muscle cells to IL-1 beta. Am J. Physiol. 1999 November; 277(5 Pt 1):L943-51.
¹⁰⁶ Larsen C M, Wadt K A, Juhl L F, Andersen H U, Karlsen A E, Su M S, Seedorf K, Shapiro L, Dinarello C A, Mandrup-Poulsen T. Interleukin-1beta-induced rat pancreatic islet nitric oxide synthesis requires both the p38 and extracellular signal-regulated kinase 1/2 mitogen-activated protein kinases. J Biol. Chem. 1998 Jun. 12; 273(24): 15294-300.
¹⁰⁷ Daeipour M, Kumar G, Amaral M C, Nel A E. Recombinant IL-6 activates p42 and p44 mitogen-activated protein kinases in the IL-6 responsive B cell line, AF-10. J. Immunol. 1993 Jun. 1; 150(11):4743-53.
¹⁰⁸ Leonard M, Ryan M P, Watson A J, Schramek H, Healy E. Role of MAP kinase pathways in mediating IL-6 production in human primary mesangial and proximal tubular cells. Kidney Int. 1999 October; 56(4):1366-77.
¹⁰⁹ Hartsough M T, Mulder K M. Transforming growth factor beta activation of p44mapk in proliferating cultures of epithelial cells. J Biol. Chem. 1995 Mar. 31; 270(13):7117-24.
¹¹⁰ Yonekura A, Osaki M, Hirota Y, Tsukazaki T, Miyazaki Y, Matsumoto T, Ohtsuru A, Namba H, Shindo H, Yamashita S. Transforming growth factor-beta stimulates articular chondrocyte cell growth through p44/42 MAP kinase (ERK) activation. Endocr J. 1999 August; 46(4):545-53.
¹¹¹ Strakova Z, Copland J A, Lolait S J, Soloff M S. ERK2 mediates oxytocin-stimulated PGE2 synthesis. Am J. Physiol. 1998 April; 274(4 Pt 1):E634-41.
¹¹² Copland J A, Jeng Y J, Strakova Z, Ives K L, Hellmich M R, Soloff M S. Demonstration of functional oxytocin receptors in human breast Hs578T cells and their up-regulation through a protein kinase C-dependent pathway. Endocrinology. 1999 May; 140(5):2258-67.
¹¹³ Subramanian C, Hasan S, Rowe M, Hottiger M, Orre R, Robertson E S. Epstein-Barr virus nuclear antigen 3C and prothymosin alpha interact with the p300 transcriptional coactivator at the CH1 and CH3/HAT domains and cooperate in regulation of transcription and histone acetylation. J. Virol. 2002 May; 76(10):4699-708.
¹¹⁴ Banas B, Eberle J, Banas B, Schlondorff D, Luckow B. Modulation of HIV-1 enhancer activity and virus production by cAMP. FEBS Lett. 2001 Dec. 7; 509(2):207-12.
¹¹⁵ Deng L, de la Fuente C, Fu P, Wang L, Donnelly R, Wade J D, Lambert P, Li H, Lee C G, Kashanchi F. Acetylation of HIV-1 Tat by CBP/P300 increases transcription of integrated HIV-1 genome and enhances binding to core histones. Virology. 2000 Nov. 25; 277(2):278-95.
¹¹⁶ Cho S, Tian Y, Benjamin T L. Binding of p300/CBP co-activators by polyoma large T antigen. J Biol. Chem. 2001 Sep. 7; 276(36):33533-9.
¹¹⁷ Wong H K, Ziff E B. Complementary functions of E1a conserved region 1 cooperate with conserved region 3 to activate adenovirus serotype 5 early promoters. J. Virol. 1994 August; 68(8):4910-20.
¹¹⁸ Asano M, Murakami Y, Furukawa K, Yamaguchi-Iwai Y, Stake M, Ito Y. A Polayomavirus Enhancers Binding Protein, PEBP5, Responsive to 12-O-Tetradecanoylphorbol-13-Acetate but Distinct From AP-1. Journal of Virology 1990 64(12):5927-5938.
¹¹⁹ Higashino F, Yoshida K, Fujinaga Y, Kamio K, Fujinaga K. Isolation fo a cDNA Encoding the Adenovirus E1A Enhancer Binding Protein: A New Human Member of the ets Oncogene Family. Nucleic Acids Research 1993 21(3):547-553.
¹²⁰ Laimins L A, Tsichlis P, Khoury G. Multiple Enhancer Domains in the 3′ Terminus of the Prague Strain of Rous Sarcoma Virus. Nucleic Acids Research 1984 12(16):6427-6442.
¹²¹ LaMarco K L, McKnight S L. Purification of a set of cellular polypeptides that bind to the purine-rich cis-regulatory element of herpes simplex virus immediate early genes. Genes Dev 1989 3(9):1372-83.
¹²² Douville P, Hagmann M, Georgiev O, Schaffner W. Positive and negative regulation at the herpes simplex virus ICP4 and ICP0 TAATGARAT motifs. Virology. 1995 Feb. 20; 207(1):107-16.
¹²³ Boshart M, Weber F, Jahn G, Dorsch-Hasler K, Fleckenstein B, Schaffner W. A very strong enhancer is located upstream of an immediate early gene of human cytomegalovirus. Cell 1985 June; 41(2):521-30.
¹²⁴ Gunther C V, Graves B J. Identification of ETS domain proteins in murine T lymphocytes that interact with the Moloney murine leukemia virus enhancer. Mol Cell Biol 1994 14(11): 7569-80
¹²⁵ Flory E, Hoffmeyer A, Smola U, Rapp U R, Bruder J T. Raf-1 kinase targets GA-binding protein in transcriptional regulation of the human immunodeficiency virus type I promoter. J Virol 1996 April; 70(4):2260-8.
¹²⁶ Rawlins D R, Milman G, Hayward S D, Hayward G S. Sequence-specific DNA binding of the Epstein-Barr virus nuclear antigen (EBNA-1) to clustered sites in the plasmid maintenance region. Cell 1985 October; 42(3):859-68.
¹²⁷ Mauclere P, Mahieux R, Garcia-Calleja J M, Salla R, Tekaia F, Millan J, De The G, Gessain A. A new HTLV-II subtype A isolate in an HIV-1 infected prostitute from Cameroon, Central Africa. AIDS Res Hum Retroviruses. 1995 August; 11(8):989-93.
¹²⁸ Kornfeld H, Riedel N, Viglianti G A, Hirsch V, Mullins J I. Cloning of HTLV-4 and its relation to simian and human immunodeficiency viruses. Nature 1987 326(6113); 610-613.
¹²⁹ Bruder J T, Hearing P. Cooperative binding of EF-1A to the E1A enhancer region mediates synergistic effects on E1A transcription during adenovirus infection. J. Virol. 1991 September; 65(9):5084-7.
¹³⁰ Bruder J T, Hearing P. Nuclear factor EF-1A binds to the adenovirus E1A core enhancer element and to other transcriptional control regions. Mol Cell Biol. 1989 November; 9(11):5143-53.
¹³¹ Ostapchuk P, Diffley J F, Bruder J T, Stillman B, Levine A J, Hearing P. Interaction of a nuclear factor with the polyomavirus enhancer region. Proc Natl Acad Sci USA. 1986 November; 83(22):8550-4.
¹³² Scholer H R, Gruss P. Specific interaction between enhancer-containing molecules and cellular components. Cell. 1984 February; 36(2):403-11.
¹³³ Nuclear Respiratory Factor 2 should not be confused with NF-E2 Related Factor 2 which is also abbriviated NRF2 or NRF-2.
¹³⁴ Watanabe H, Imai T, Sharp P A, Handa H. Identification of two transcription factors that bind to specific elements in the promoter of the adenovirus early-region 4: Mol Cell Biol 1988 8(3):1290-300. The transcription factor binds to the promoter of the adenovirus early-region 4 (E4). Hence the name E4 transcription factor 1.
¹³⁵ Enhancer Factor 1A should not be confused with Elongation Factor 1A which is also abbriviated EF-1A.
¹³⁶ Suzuki F, Goto M, Sawa C, Ito S, Watanabe H, Sawada J, Handa H. Functional interactions of transcription factor human GA-binding protein subunits. J Biol. Chem. 1998 Nov. 6; 273(45):29302-8.
¹³⁷ Sambrok J, MacCallum P, Russell D, eds. Molecular Cloning: A Laboratory Manual, 3rd Edition. Cold Spring Harbor Laboratory Press., 2001.
¹³⁸ Ausubel, et al., ets. Current Protocols in Molecular Biology. NY: John Wiley & Sons. 1998.
¹³⁹ Friedmann T, ed. The Development of Human Gene Therapy, Cold Spring Harbor Press, 1999.
¹⁴⁰ Jones P, Ramji D, Gacesa P, eds. Vectors: Expression Systems: Essential Techniques. (John Wiley and Sons, 1998
¹⁴¹ Deshmukh, R. R., Cole, D. L. and Sanghvi, Y. S. Purification of Antisense Oligonucleotides in Methods in Enzymology, ed. M. Ian Phillips, v313, 1999, Academic Press, pp203-226.
¹⁴² Daftary, G. S. and Taylor, H. S. Efficient liposome-mediated gene transfection and expression in the intact human uterus. Hum Gene Ther 12,2121-2127 2001. Yale University School of Medicine, New Haven, Conn. 06520-8063, USA.
¹⁴³ Doherty, E. A and Doudna. Ribozyme structures and mechanisms. J.A. Ann. Rev. Biophys. Biomol Struct. 30, 457-475, 2001.
¹⁴⁴ Goodchild, J. Hamerhead ribozymes: biochemical and chemical considerations. Curr. Opin. Mol. Ther 2, 272-281, 2000. Review.
¹⁴⁵ Francois, J.-C., Lacoste, J., Lacroix, L. and J.-L. Mergny. Design of Antisense and Triplex-Forming Oligonucleotides. In Methods in Enzymology, ed. M Ian Phillips, v313, 1999, Academic Press, pp74-95
¹⁴⁶ Hyrup, B and Nielseni P. E. Peptide nucleic acids (PNA): synthesis, properties and potential applications. Bioorg. Med. Chem. 4: 5-23, 1996
¹⁴⁷ Perry-O'Keefe, H., Yao, X. W, Coull, J. M., Fuchs, M. and Egholm, M. Peptide nucleic acid pre-gel hybridization: an alternative to Southern hybridization. Proc. Natl. Acad. Sci. USA 93: 14670-14675, 1996.
¹⁴⁸ Nielsen, P. E. (1999) Antisense Properties of Peptide Nucleic Acid. In Methods in Enzymology, ed. M. Ian Phillips, v313. 1999, Academic Press Academic Press, pp156-164.
¹⁴⁹ Harlow and Lane, eds. Using Antibodies: A Laboratory Manual. Cold Spring Harbor Press, 1999.
¹⁵⁰ Sambrook J, Fritsch E F, and Maniatis T. Molecular Cloning, Cold Spring Harbor Press, 1989.
¹⁵¹ Kohler G, Milstein C. Continuous cultures of fused cells secreting antibody of predefined specificity. Nature 256: 495-497, 1975.
¹⁵² Zola H. Monoclonal Antibodies: Preparation and Use of Monoclonal Antibodies and Engineered Antibody Derivatives (Basics: From Background to Bench), 2000.
¹⁵³ Ausubel F M, Brent R, Kingston R E, Moore D D, Seidman J G, Smith J A, Struhl K. Short protocols in molecular biology (4th ed.), John Wiley and Sons, Inc., 1999.
¹⁵⁴ Sapan C V, Lundblad R L, Price N C. Colorimetric protein assay techniques. Biotechnol Appl Biochem 29, 99-108, 1999.
¹⁵⁵ Manchester K L. Value of A260/A280 ratios for measurement of purity of nucleic acids. Biotechniques 19, 208-210, 1995.
¹⁵⁶ Davis L G, Dibner M D, Battey J F. Basic methods in molecular biology, Elsevier Science Publishing Co., Inc. 1986.
¹⁵⁷ Gizard F, Lavallee B. DeWitte F, Hum D W. A novel zinc finger protein TreP-132 interacts with CBP/p300 to regulate human p450scc gene expression. J. Biol. Chem. May 2001, in press.
¹⁵⁸ Heid C A, Stevens J, Livak K J, Williams, P M. Real time quantitative PCR. Genome Res. 6, 986-994, 1996.
¹⁵⁹ Nuchprayoon I, Shang J, Simkevich C P, Luo M, Rosmarin A G, Friedman A D. An enhancer located between the neutrophil elastase and proteinase 3 promoters is activated by Sp1 and an Ets Factor. J. Biol. Chem. 274, 1085-1091, 1999.
¹⁶⁰ Creighton, 1983, Proteins: Structures and Molecular Principles, W. H. Freeman & Co., NY, pp. 34-49
¹⁶¹ Sanger F, Nicklen S, Coulson A R. DNA sequencing with chain-terminating inhibitors. Proc. Natl. Acad. Sci. USA 74, 5463-5467, 1977.
¹⁶² Kristensen V N, Kelefiotis D, Kristensen T, Borresen-Dale A L. High-throughput methods for detection of genetic variation. Biotechniques 2001 February; 30(2):318-22, 324, 326 passim.
¹⁶³ Tawata M, Aida K, Onaya T. Screening for genetic mutations. A review. Comb Chem High Throughput Screen. 2000 February; 3(1):1-9. Review.
¹⁶⁴ Pecheniuk N M, Walsh T P, Marsh N A. DNA technology for the detection of common genetic variants that predispose to thrombophilia. Blood Coagul Fibrinolysis 2000 December; 11(8):683-700.
¹⁶⁵ Coffon R G. Current methods of mutation detection. Mutat Res. 1993 January; 285(1):125-44. Review.
¹⁶⁶ Prosser J. Detecting single-base mutations. Trends Biotechnol. 1993 June; 11(6):238-46. Review.
¹⁶⁷ Abrams E S, Murdaugh S E, Lerman L S. Comprehensive detection of single base changes in human genomic DNA using denaturing gradient gel electrophoresis and a GC clamp. Genomics. 1990 August; 7(4):463-75.
¹⁶⁸ Forrest S, Cotton R G. Methods of detection of single base substitutions in clinical genetic practice. Mol Biol Med. 1990 October; 7(5):451-9. Review.
¹⁶⁹ Graham C A, Hill A J. Introduction to DNA sequencing. Methods Mol. Biol. 2001; 167:1-12. Review.
¹⁷⁰ Rapley R. eds. PCR sequencing protocols. Humana Press, Totowa, N.J., 1996.
¹⁷¹ Marziali A, Akeson M. New DNA sequencing methods. Annu Rev Biomed Eng 2001; 3:195-223.
¹⁷² Dovichi N J, Zhang J. DNA sequencing by capillary array electrophoresis. Methods Mol Biol 2001; 167:225-39. Review.
¹⁷³Huang G M. High-throughput DNA sequencing: a genomic data manufacturing process. DNA Seq. 1999; 10(3):149-53. Review.
¹⁷⁴ Schmalzing D, Koutny L, Salas-Solano O, Adourian A, Matsudaira P, Ehrlich D. Recent developments in DNA sequencing by capillary and microdevice electrophoresis. Electrophoresis. 1999 October; 20(15-16):3066-77. Review.
¹⁷⁵ Murray K K. DNA sequencing by mass spectrometry. J Mass Spectrom. 1996 November; 31(11):1203-15.
¹⁷⁶ Cohen A S, Smisek D L, Wang B H. Emerging technologies for sequencing antisense oligonucleotides: capillary electrophoresis and mass spectrometry. Adv Chromatogr. 1996; 36:127-62. Review.
¹⁷⁷ Griffin H G, Griffin A M. DNA sequencing. Recent innovations and future trends. Appl Biochem Biotechnol. 1993 January-February; 38(1-2):147-59. Review.
¹⁷⁸ Watts D, MacBeath J R. Automated fluorescent DNA sequencing on the ABI PRISM 310 Genetic Analyzer. Methods Mol. Biol. 2001; 167:153-70. Review.
¹⁷⁹ MacBeath J R, Harvey S S, Oldroyd N J. Automated fluorescent DNA sequencing on the ABI PRISM 377. Methods Mol. Biol. 2001; 167:119-52. Review.
¹⁸⁰ Smith L M, Brumley R L Jr, Buxton E C, Giddings M, Marchbanks M, Tong X. High-speed automated DNA sequencing in ultrathin slab gels. Methods Enzymol. 1996; 271:219-37. Review.
¹⁸¹ Maxam A M, Gilbert W. A new method for sequencing DNA. Proc Natl Acad Sci USA. 1977 February; 74(2):560-4.
¹⁸² Saleeba J A, Cotton R G. Chemical cleavage of mismatch to detect mutations. Methods Enzymol. 1993; 217:286-95.
¹⁸³ Takahashi N, Hiyama K, Kodaira M, Satoh C. An improved method for the detection of genetic variations in DNA with denaturing gradient gel electrophoresis. Mutat Res. 1990 April; 234(2):61-70.
¹⁸⁴ Cotton R G, Rodrigues N R, Campbell R D. Reactivity of cytosine and thymine in single-base-pair mismatches with hydroxylamine and osmium tetroxide and its application to the study of mutations. Proc Natl Acad Sci USA. 1988 June; 85(12):4397-401.
¹⁸⁵ Myers R M, Larin Z, Maniatis T. Detection of single base substitutions by ribonuclease cleavage at mismatches in RNA:DNA duplexes. Science. 1985 Dec. 13; 230(4731):1242-6.
¹⁸⁶ Myers R M, Lumelsky N, Lerman L S, Maniatis T. Detection of single base substitutions in total genomic DNA. Nature. 1985 Feb. 7-13; 313(6002):495-8.
¹⁸⁷ Xu J F, Yang Q P, Chen J Y, van Baalen M R, Hsu I C. Determining the site and nature of DNA mutations with the cloned MutY mismatch repair enzyme. Carcinogenesis. 1996 February; 17(2):321-6.
¹⁸⁸ Hsu I C, Yang Q, Kahng M W, Xu J F. Detection of DNA point mutations with DNA mismatch repair enzymes. Carcinogenesis. 1994 August; 15(8): 1657-62.
¹⁸⁹ Miterski B. Kruger R, Wintermeyer P, Epplen J T. PCR/SSCP detects reliably and efficiently DNA sequence variations in large scale screening projects. Comb Chem High Throughput Screen 2000 June; 3(3):211-8.
¹⁹⁰ Jaeckel S, Epplen J T, Kauth M, Miterski B, Tschentscher F, Epplen C. Polymerase chain reaction-single strand conformation polymorphism or how to detect reliably and efficiently each sequence variation in many samples and many genes. Electrophoresis. 1998 December; 19(18):3055-61. Review.
¹⁹¹ Hayashi K. PCR-SSCP: a method for detection of mutations. Genet Anal Tech Appl. 1992 June; 9(3):73-9. Review.
¹⁹² Lipshutz R J, Morris D, Chee M, Hubbell E, Kozal M J, Shah N, Shen N, Yang R, Fodor S P. Using oligonucleotide probe arrays to access genetic diversity. Biotechniques 1995 September; 19(3):442-7.
¹⁹³ Guo Z, Guilfoyle R A, Thiel A J, Wang R. Smith L M. Direct fluorescence analysis of genetic polymorphisms by hybridization with oligonucleotide arrays on glass supports. Nucleic Acids Res 1994 Dec. 11; 22(24):5456-65.
¹⁹⁴ Saiki R K, Walsh P S, Levenson C H, Erlich H A. Genetic analysis of amplified DNA with immobilized sequence-specific oligonucleotide probes. Proc Natl Acad Sci USA. 1989 August; 86(16):6230-4.
¹⁹⁵ Efremov D G, Dimovski A J, Jankovic L, Efremov G D. Mutant oligonucleotide extension amplification: a nonlabeling polymerase-chain-reaction-based assay for the detection of point mutations. Acta Haematol 1991; 85(2):66-70.
¹⁹⁶ Gibbs R A, Nguyen P N, Caskey C T. Detection of single DNA base differences by competitive oligonucleotide priming. Nucleic Acids Res. 1989 Apr 11; 17(7):2437-48.
¹⁹⁷ Geisler J P, Hatterman-Zogg M A, Rathe J A, Lallas T A, Kirby P, Buller R E. Ovarian cancer BRCA1 mutation detection: Protein truncation test (PTT) outperforms single strand conformation polymorphism analysis (SSCP). Hum Mutat. 2001 October; 18(4):337-44.
¹⁹⁸ Moore W, Bogdarina I, Patel U A, Perry M, Crane-Robinson C. Mutation detection in the breast cancer gene BRCA1 using the protein truncation test. Mol Biotechnol. 2000 February; 14(2):89-97.
¹⁹⁹ van der Luijt R, Khan P M, Vasen H, van Leeuwen C, Tops C, Roest P, den Dunnen J, Fodde R. Rapid detection of translation-terminating mutations at the adenomatous polyposis coli (APC) gene by direct protein truncation test. Genomics. 1994 Mar. 1; 20(1):1-4.
²⁰⁰ Roest P A, Roberts R G, Sugino S, van Ommen G J, den Dunnen J T. Protein truncation test (PTT) for rapid detection of translation-terminating mutations. Hum Mol Genet. 1993 October; 2(10):1719-21.
²⁰¹ Burnett W N. Western blotting electrophoretic transfer of proteins from SDS-polyacrylamide to unmodified nitrocellulose and autoradiographic detection with antibody and radioiodinated protein A. Ann Biochem, 112:195-203 1981.
²⁰² Virts E L, Raschke W C. The Role of Intron Sequences in High Level Expression from CD45 cDNA Constructs. J Biol Chem, 276, 19913-19920, 2001.
²⁰³ Chen C, Okayama H. Calcium phosphate-mediated gene transfer: A highly efficient system for stably transforming cells with plasmid DNA. BioTech. 6, 632-638, 1988.
²⁰⁴ Lopata M A, Cleveland D W, Sollner-Webb B. High-level expression of a chloramphenicol acetyltransferase gene by DEAE-dextran-mediated DNA transfection coupled with a dimethyl sulfoxide or glycerol shock treatment. Nucl. Acids Res. 12, 5707-5717, 1984.
²⁰⁵ Gorman C M, Moffat L F, Howard B H. Recombinant genomes which express chloramphenicol acetyltransferase in mammalian cells. Mol. Cell. Biol. 2, 1044-1051, 1982.
²⁰⁶ Luo R Z, Peng H, Xu F, Bao J, Pang Y, Pershad R, Issa J J, Liao W S, Bast R C, Yu Y. Genomic structure and promoter characterization of an imprinted tumor suppressor gene ARH1(1). Biochim Biophys Acta, 1519, 216-222, 2001.
²⁰⁷ Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF 1. Cancer Res. 57, 3145-3148, 1997.
²⁰⁸ Sucharov C, Basu A, Carter R S, Avadhani N G. A novel transcriptional initiator activity of the GABP factor binding ets sequence repeat from the murine cytochrome c oxidase Vb gene. Gene Expr. 5, 93-111, 1995.
²⁰⁹ Ouyang L, Jacob K K, Stanley F M. GABP mediates insulin-increased prolactin gene transcription. J. Biol. Chem. 271, 10425-10428, 1.996.
²¹⁰ Staskus K A, Embretson J E, Retzel E F, Beneke J, Haase A T. PCR in situ: new technologies with single cell resolution for the detection and investigation of viral latency and persistence.
http://www.cbc.umn.edu/VirtLibrary/Staskus/chap-shoot2.fm.html, 1994.
²¹¹ Schuurhuis G J, Muijen M M, Oberink J W, de Boer F, Ossenkoppele G J, Broxterman H J. Large populations of non-clonogenic early apoptotic CD34-positive cells are present in frozen-thawed peripheral blood stem cell transplants. Bone Marrow Transplant 27, 487-498, 200.1.
²¹² Weyers A, Gorla N, Ugnia L, Garcia Ovando H, Chesta C. Increase of tissue lipid hydroperoxides as determination of oxidative stress. Biocell 25, 11-5 2001.
²¹³ Brubacher J L, Bols N C. Chemically de-acetylated 2′,7′-dichlorodihydrofluorescein diacetate as a probe of respiratory burst activity in mononuclear phagocytes. J. Immunol. Tethods 25, 81-91, 2001.
²¹⁴ Watson J D, Crick F H C. Molecular Structure of Nucleic Acid. Nature, 1953, 737-738.
²¹⁵ Friedman M, Friedland G W. Medicine's 10 greatest discoveries. Yale University Press. 1998.
²¹⁶ Einstein A. Autobiographical notes. In, Schilpp P A (ed). Albert Einstein: Philosopher-Scientist. Tudor Publishing Company, NY. 1951.
²¹⁷ Scholer H R, Gruss P. Specific interaction between enhancer-containing molecules and cellular components. Cell. 1984 February; 36(2):403-11.
²¹⁸ Mercola M, Goverman J, Mirell C, Calame K. Immunoglobulin heavy-chain enhancer requires one or more tissue-specific factors. Science. 1985 Jan. 18; 227(4684):266-70.
²¹⁹ Scholer H, Haslinger A, Heguy A, Holtgreve H, Karin M. In Vivo Competition Between a Metallothionein Regulatory Element and the SV40 Enhancer. Science 1986 232: 76-80.
²²⁰ Adam G I, Miller S J, Ulleras E, Franklin G C. Cell-type-specific modulation of PDGF-B regulatory elements via viral enhancer competition: a caveat for the use of reference plasmids in transient transfection assays. Gene. 1996 Oct. 31; 178(1-2):25-9.
²²¹ Hofman K, Swinnen J V, Claessens F, Verhoeven G, Heyns W. Apparent coactivation due to interference of expression constructs with nuclear receptor expression. Mol Cell Endocrinol. 2000 Oct. 25; 168(1-2):21-9.
²²² Watanabe H, Imai T, Sharp P A, Handa H. Identification of two transcription factors that bind to specific elements in the promoter of the adenovirus early-region 4. Mol Cell Biol 1988 8(3): 1290-300.
²²³ Suzuki F, Goto M, Sawa C, Ito S, Watanabe H, Sawada J, Handa H. Functional interactions of transcription factor human GA-binding protein subunits. J Biol. Chem. 1998 Nov. 6; 273(45):29302-8.
²²⁴ Rosmarin A G, Luo M, Caprio D G, Shang J, Simkevich C P. Sp1 cooperates with the ets transcription factor, GABP, to activate the CD18 (beta2 leukocyte integrin) promoter. J Biol. Chem. 1998 May 22; 273(21): 13097-103.
²²⁵ Bannert R, Avots A, Baier M, Serfling E, Kurth R. GA-binding protein factors, in concert with the coactivator CREB binding protein/p300, control the induction of the interleukin 16 promoter in T lymphocytes. Proc. Natl. Acad. Sci. USA 1999 96:1541-1546.
²²⁶ Avots A, Hoffmeyer A, Flory E, Cimanis A, Rapp U R, Serfling E. GABP factors bind to a distal interleukin 2 (IL-2) enhancer and contribute to c-Raf-mediated increase in IL-2 induction. Molecular and Cellular Biology 1997 17(8):4381-4389.
²²⁷ Lin J X, Bhat N K, John S, Queale W S, Leonard W J. Characterization of the human interleukin-2 receptor beta-chain gene promoter: regulation of promoter activity by ets gene products. Mol Cell Biol. 1993 October; 13(10):6201-10.
²²⁸ Markiewicz S, Bosselut R, Le Deist F, de Villartay J P, Hivroz C, Ghysdael J, Fischer A, de Saint Basile G. Tissue-specific activity of the gammac chain gene promoter depends upon an Ets binding site and is regulated by GA-binding protein. J Biol. Chem. 1996 Jun. 21; 271(25):14849-55.
²²⁹ Smith M F Jr, Carl V S, Lodie T, Fenton M J. Secretory interleukin-1 receptor antagonist gene expression requires both a PU.1 and a novel composite NF-kappaB/PU.1/GA-binding protein binding site. J Biol. Chem. 1998 Sep. 11; 273(37):24272-9.
²³⁰ Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. 1997 Aug. 1; 57(15):3145-8.
²³¹ Kamura T, Handa H, Hamasaki N, Kitajima S. Characterization of the human thrombopoietin gene promoter. A possible role of an Ets transcription factor, E4TF1/GABP. J Biol. Chem. 1997 Apr. 25; 272(17):11361-8.
²³² Wang K, Bohren K M, Gabbay K H. Characterization of the human aldose reductase gene promoter. J Biol. Chem. 1993 Jul. 25; 268(21):16052-8.
²³³ Nuchprayoon I, Shang J, Simkevich C P, Luo M, Rosmarin A G, Friedman A D. An enhancer located between the neutrophil elastase and proteinase 3 promoters is activated by Sp1 and an Ets factor. J Biol. Chem. 1999 Jan. 8; 274(2): 1085-91.
²³⁴ Nuchprayoon 1, Simkevich C P, Luo M, Friedman A D, Rosmarin A G. GABP cooperates with c-Myb and C/EBP to activate the neutrophil elastase promoter. Blood. 1997 Jun. 15; 89(12):4546-54.
²³⁵ Sadasivan E, Cedeno M M, Rothenberg S P. Characterization of the gene encoding a folate-binding protein expressed in human placenta. Identification of promoter activity in a G-rich SP1 site linked with the tandemly repeated GGAAG motif for the ets encoded GA-binding protein. J Biol. Chem. 1994 Feb. 18; 269(7):4725-35.
²³⁶ Basu A, Park K, Atchison M L, Carter R S, Avadhani N G. Identification of a transcriptional initiator element in the cytochrome c oxidase subunit Vb promoter which binds to transcription factors NF-E1 (YY-1, delta) and S21. J Biol. Chem. 1993 Feb. 25; 268(6):4188-96.
²³⁷ Sucharov C, Basu A, Carter R S, Avadhani N G. A novel transcriptional initiator activity of the GABP factor binding ets sequence repeat from the murine cytochrome c oxidase Vb gene. Gene Expr. 1995; 5(2):93-111.
²³⁸ Carter R S, Avadhani N G. Cooperative binding of GA-binding protein transcription factors to duplicated transcription initiation region repeats of the cytochrome c oxidase subunit IV gene. J Biol. Chem. 1994 Feb. 11; 269(6):4381-7.
²³⁹ Carter R S, Bhat N K, Basu A, Avadhani N G. The basal promoter elements of murine cytochrome c oxidase subunit IV gene consist of tandemly duplicated ets motifs that bind to GABP-related transcription factors. J Biol. Chem. 1992 Nov. 15; 267(32):23418-26.
²⁴⁰ Virbasius J V, Scarpulla R C. Activation of the human mitochondrial transcription factor A gene by nuclear respiratory factors: a potential regulatory link between nuclear and mitochondrial gene expression in organelle biogenesis. Proc Natl Acad Sci USA. 1994 Feb. 15; 91(4):1309-13.
²⁴¹ Villena J A, Vinas O, Mampel T, Iglesias R, Giralt M, Villarroya F. Regulation of mitochondrial biogenesis in brown adipose tissue: nuclear respiratory factor-2/GA-binding protein is responsible for the transcriptional regulation of the gene for the mitochondrial ATP synthase beta subunit. Biochem J. 1998 Apr. 1; 331 (Pt 1): 121-7.
²⁴² Ouyang L, Jacob K K, Stanley F M. GABP mediates insulin-increased prolactin gene transcription. J Biol Chem. 1996 May 3; 271(18):10425-8.
²⁴³ Hoare S, Copland J A, Wood T G, Jeng Y J, Izban M G, Soloff M S. Identification of a GABP alpha/beta binding site involved in the induction of oxytocin receptor gene expression in human breast cells, potentiation by c-Fos/c-Jun. Endocrinology. 1999 May; 140(5):2268-79.
²⁴⁴ Asano M, Murakami Y, Furukawa K, Yamaguchi-Iwai Y, Stake M, Ito Y. A Polayomavirus Enhancers Binding Protein, PEBP5, Responsive to 12-O-Tetradecanoylphorbol-13-Acetate but Distinct From AP-1. Journal of Virology 1990 64(12):5927-5938.
²⁴⁵ Higashino F, Yoshida K, Fujinaga Y, Kamio K, Fujinaga K. Isolation fo a cDNA Encoding the Adenovirus E1A Enhancer Binding Protein: A New Human Member of the ets Oncogene Family. Nucleic Acids Research 1993 21(3):547-553.
²⁴⁶ Laimins L A, Tsichlis P, Khoury G. Multiple Enhancer Domains in the 3′ Terminus of the Prague Strain of Rous Sarcoma Virus. Nucleic Acids Research 1984 12(16):6427-6442.
²⁴⁷ LaMarco K L, McKnight S L. Purification of a set of cellular polypeptides that bind to the purine-rich cis-regulatory element of herpes simplex virus immediate early genes. Genes Dev 1989 3(9):1372-83.
²⁴⁸ Douville P, Hagmann M, Georgiev O, Schaffner W. Positive and negative regulation at the herpes simplex virus ICP4 and ICP0 TAATGARAT motifs. Virology. 1995 Feb. 20; 207(1):1.07-16.
²⁴⁹ Boshart M, Weber F, Jahn G, Dorsch-Hasler K, Fleckenstein B, Schaffner W. A very strong enhancer is located upstream of an immediate early gene of human cytomegalovirus. Cell 1985 June; 41(2):521-30.
²⁵⁰ Gunther C V, Graves B J. Identification of ETS domain proteins in murine T lymphocytes that interact with the Moloney murine leukemia virus enhancer. Mol Cell Biol 1994 14(11): 7569-80
²⁵¹ Flory E, Hoffmeyer A, Smola U, Rapp U R, Bruder J T. Raf-1 kinase targets GA-binding protein in transcriptional regulation of the human immunodeficiency virus type I promoter. J Virol 1996 April; 70(4):2260-8.
²⁵² Rawlins D R, Milman G, Hayward S D, Hayward G S. Sequence-specific DNA binding of the Epstein-Barr virus nuclear antigen (EBNA-1) to clustered sites in the plasmid maintenance region. Cell 1985 October; 42(3):859-68.
²⁵³ Mauclere P, Mahieux R, Garcia-Calleja J M, Salia R, Tekaia F, Millan J, De The G, Gessain A. A new HTLV-II subtype A isolate in an HIV-1 infected prostitute from Cameroon, Central Africa. AIDS Res Hum Retroviruses. 1995 August; 11(8):989-93.
²⁵⁴ Kornfeld H, Riedel N, Viglianti G A, Hirsch V, Mullins J I. Cloning of HTLV-4 and its relation to simian and human immunodeficiency viruses. Nature 1987 326(6113); 610-613.
²⁵⁵ Bruder J T, Hearing P. Cooperative binding of EF-1A to the E1A enhancer region mediates synergistic effects on E1A transcription during adenovirus infection. J. Virol. 1991 September; 65(9):5084-7.
²⁵⁶ Bruder J T, Hearing P. Nuclear factor EF-1A binds to the adenovirus E1A core enhancer element and to other transcriptional control regions. Mol Cell Biol. 1989 November; 9(11):5143-53.
²⁵⁷ Ostapchuk P, Diffley J F, Bruder J T, Stillman B, Levine A J, Hearing P. Interaction of a nuclear factor with the polyomavirus enhancer region. Proc Natl Acad Sci USA. 1986 November; 83(22):8550-4.
²⁵⁸ Kamei Y, Xu L, Heinzel T, Torchia J, Kurokawa R, Gloss B, Lin S C, Heyman R A, Rose D W, Glass C K, Rosenfeld M G. A CBP integrator complex mediates transcriptional activation and AP-1 inhibition by nuclear receptors. Cell. 1996 May 3; 85(3):403-14.
²⁵⁹ Horvai A E, Xu L, Korzus E, Brard G, Kalafus D, Mullen T M, Rose D W, Rosenfeld M G, Glass C K. Nuclear integration of JAK/STAT and Ras/AP-1 signaling by CBP and p300. Proc Natl Acad Sci USA. 1997 Feb. 18; 94(4):1074-9.
²⁶⁰ Hottiger M O, Felzien L K, Nabel G J. Modulation of cytokine-induced HIV gene expression by competitive binding of transcription factors to the coactivator p300. EMBO J. 1998 Jun. 1; 17(11):3124-34.
²⁶¹ Pise-Masison C A, Mahieux R, Radonovich M, Jiang H, Brady J N. Human T-lymphotropic virus type I Tax protein utilizes distinct pathways for p53 inhibition that are cell type-dependent. J Biol. Chem. 2001 Jan. 5; 276(1):200-5.
²⁶² Banas B, Eberle J, Banas B, Schlondorff D, Luckow B. Modulation of HIV-1 enhancer activity and virus production by cAMP. FEBS Lett. 2001 Dec. 7; 509(2):207-12.
²⁶³ Wang C, Fu M, D'Amico M, Albanese C, Zhou J N, Brownlee M, Lisanti M P, Chatterjee V K, Lazar M A, Pestell R G. Inhibition of cellular proliferation through IkappaB kinase-independent and peroxisome proliferator-activated receptor gamma-dependent repression of cyclin D1. Mol Cell Biol. 2001 May; 21(9):3057-70.
²⁶⁴ Ernst P, Wang J, Huang M, Goodman R H, Korsmeyer S J. MLL and CREB bind cooperatively to the nuclear coactivator CREB-binding protein. Mol Cell Biol. 2001 April; 21(7):2249-58.
²⁶⁵ Yuan W, Varga J. Transforming growth factor-beta repression of matrix metalloproteinase-1 in dermal fibroblasts involves Smad3. J Biol. Chem. 2001 Oct. 19; 276(42):38502-10.
²⁶⁶ Ghosh A K, Yuan W, Mori Y, Chen Sj, Varga J. Antagonistic regulation of type I collagen gene expression by interferon-gamma and transforming growth factor-beta. Integration at the level of p300/CBP transcriptional coactivators. J Biol. Chem. 2001 Apr. 6; 276(14):11041-8.
²⁶⁷ Li M, Pascual G, Glass C K. Peroxisome proliferator-activated receptor gamma-dependent repression of the inducible nitric oxide synthase gene. Mol Cell Biol. 2000 July; 20(13):4699-707.
²⁶⁸ Nagarajan R P, Chen F, Li W, Vig E, Harrington M A, Nakshatri H, Chen Y. Repression of transforming-growth-factor-beta-mediated transcription by nuclear factor kappaB. Biochem J. 2000 Jun. 15; 348 Pt 3:591-6.
²⁶⁹ Speir E, Yu Z X, Takeda K, Ferrans V J, Cannon R O 3rd. Competition for p300 regulates transcription by estrogen receptors and nuclear factor-kappaB in human coronary smooth muscle cells. Circ Res. 2000 Nov. 24; 87(11):1006-11.
²⁷⁰ Chen Y H, Ramos K S. A CCAAT/enhancer-binding protein site within antioxidant/electrophile response element along with CREB-binding protein participate in the negative regulation of rat GST-Ya gene in vascular smooth muscle cells. J Biol. Chem. 2000 Sep. 1; 275(35):27366-76.
²⁷¹ Werner F, Jain M K, Feinberg M W, Sibinga N E, Pellacani A, Wiesel P, Chin M T, Topper J N, Perrella M A, Lee M E. Transforming growth factor-beta 1 inhibition of macrophage activation is mediated via Smad3. J Biol. Chem. 2000 Nov. 24; 275(47):36653-8.
²⁷² Bush T S, St Coeur M, Resendes K K, Rosmarin A G. GA-binding protein (GABP) and Sp1 are required, along with retinoid receptors, to mediate retinoic acid responsiveness of CD18 (beta 2 leukocyte integrin): a novel mechanism of transcriptional regulation in myeloid cells. Blood. 2003 Jan. 1; 101(1):311-7.
²⁷³ Rosmarin A G, Caprio D, Levy R, Simkevich C. CD18 (beta 2 leukocyte integrin) promoter requires PU.1 transcription factor for myeloid activity. Proc Natl Acad Sci USA. 1995 Jan. 31; 92(3):801-5.
²⁷⁴ Li S L, Schlegel W, Valente A J, Clark R A. Critical flanking sequences of PU.1 binding sites in myeloid-specific promoters. J Biol. Chem. 1999 Nov. 5; 274(45):32453-60.
²⁷⁵ Panopoulos A D, Bartos D, Zhang L, Watowich S S. Control of myeloid-specific integrin alpha Mbeta 2 (CD11b/CD18) expression by cytokines is regulated by Stat3-dependent activation of PU.1. J Biol Chem. 2002 May 24; 277(21):19001-7.
²⁷⁶ Rosmarin A G, Caprio D G, Kirsch D G, Handa H. Simkevich C P. GABP and PU.1 compete for binding, yet cooperate to increase CD18 (beta 2 leukocyte integrin) transcription. J Biol. Chem. 1995 Oct. 6; 270(40):23627-33.
²⁷⁷ Niwa H, Yamamura K, Miyazaki J. Efficient selection for high-expression transfectants with a novel eukaryotic vector. Gene. 1991 Dec. 15; 108(2):193-9.
²⁷⁸ Muller S, Maas A, Islam T C, Sideras P, Suske G, Philipsen S, Xanthopoulos K G, Hendriks R W, Smith C I. Synergistic activation of the human Btk promoter by transcription factors Sp1/3 and PU.1. Biochem Biophys Res Commun. 1999 Jun. 7; 259(2):364-9.
²⁷⁹ Bottinger E P, Shelley C S, Farokhzad O C, Arnaout M A. The human beta 2 integrin CD18 promoter consists of two inverted Ets cis elements. Mol Cell Biol. 1994 April; 14(4):2604-15.
²⁸⁰ Anderson K L, Smith K A, Perkin H I Hermanson G, Anderson C G, Jolly D J, Maki R A, Torbett B E. PU.1 and the granulocyte- and macrophage colony-stimulating factor receptors play distinct roles in late-stage mycloid cell differentiation. Blood. 1999 Oct. 1; 94(7):2310-8.
²⁸¹ DeKoter R P, Walsh J C, Singh H. PU.1 regulates both cytokine-dependent proliferation and differentiation of granulocyte/macrophage progenitors. EMBO J. 1998 Aug. 3; 17(15):4456-68.
²² Anderson K L, Smith K A, Conners K, McKercher S R, Maki R A, Torbett B E. Myeloid development is selectively disrupted in PU.1 null mice. Blood. 1998 May 15; 91(10):3702-10.
²⁸³ Anderson K L, Perkin H, Surh C D, Venturini S, Maki R A, Torbett B E. Transcription factor PU.1 is necessary for development of thymic and myeloid progenitor-derived dendritic cells. J. Immunol. 2000 Feb. 15,164(4):1855-61.
²⁸⁴ Guerriero A, Langmuir P B, Spain L M, Scott E W. PU.1 is required for myeloid-derived but not lymphoid-derived dendritic cells. Blood. 2000 Feb. 1; 95(3):879-85.
²⁸⁵ Cheng T, Shen H, Giokas D, Gere J, Tenen D G, Scadden D T. Temporal mapping of gene expression levels during the differentiation of individual primary hematopoietic cells. Proc Natl Acad Sci USA. 1996 Nov. 12; 93(23): 13158-63.
²⁸⁶ Voso M T, Burn T C, Wulf G, Lim B, Leone G, Tenen D G. Inhibition of hematopoiesis by competitive binding of transcription factor PU.1. Proc Natl Acad Sci USA. 1994 Aug. 16; 91(17):7932-6.
²⁸⁷ Chinenov Y, Schmidt T, Yang X Y, Martin M E. Identification of redox-sensitive cysteines in GA-binding protein-alpha that regulate DNA binding and heterodimerization. J Biol. Chem. 1998 Mar. 13; 273(11):6203-9.
²⁸⁸ Islam M R, Fan C, Fujii Y, Hao L J, Suzuki S, Kumatori A, Yang D, Rusvai E, Suzuki N, Kikuchi H, Nakamura M. PU.1 is dominant and HAF-1 supplementary for activation of the gp91 (phox) promoter in human monocytic PLB-985 cells. J Biochem (Tokyo). 2002 April; 131(4):533-40.
²⁸⁹ Voo K S, Skalnik D G. Elf-1 and PU.1 induce expression of gp91 (phox) via a promoter element mutated in a subset of chronic granulomatous disease patients. Blood. 1999 May 15; 93(10):3512-20.
²⁹⁰ Suzuki S, Kumatori A, Haagen I A, Fujii Y, Sadat M A, Jun H L, Tsuji Y, Roos D, Nakamura M. PU.1 as an essential activator for the expression of gp91 (phox) gene in human peripheral neutrophils, monocytes, and B lymphocytes. Proc Natl Acad Sci USA. 1998 May 26; 95(11):6085-90.
²⁹¹ Kalinina N, Agrotis A, Tararak E, Antropova Y, Kanellakis P, Ilyinskaya O, Quinn M T, Smirnov V, Bobik A. Cytochrome b558-dependent NAD(P)H oxidase-phox units in smooth muscle and macrophages of atherosclerotic lesions. Arterioscler Thromb Vasc Biol. 2002 Dec. 1; 22(12):2037-43.
²⁹² Rosen G D, Barks J L, Iademarco M F, Fisher R J, Dean D C. An intricate arrangement of binding sites for the Ets family of transcription factors regulates activity of the alpha 4 integrin gene promoter. J Biol. Chem. 1994 Jun. 3; 269(22): 15652-60.
²⁹³ DiMilla P A, Barbee K, Lauffenburger D A. Mathematical model for the effects of adhesion and mechanics on cell migration speed. Biophys J. 1991 July; 60(1):15-37.
²⁹⁴ Palecek S P, Loftus J C, Ginsberg M H, Lauffenburger D A, Horwitz A F. Integrin-ligand binding properties govern cell migration speed through cell-substratum adhesiveness. Nature. 1997 Feb. 6; 385(6616):537-40.
²⁹⁵ Parkhurst M R, Saltzman W M. Quantification of human neutrophil motility in three-dimensional collagen gels. Effect of collagen concentration. Biophys J. 1992 February; 61(2):306-15.
²⁹⁶ Palecek S P, Huttenlocher A, Horwitz A F, Lauffenburger D A. Physical and biochemical regulation of integrin release during rear detachment of migrating cells. J Cell Sci. 1998 April; 111 (Pt 7):929-40.
²⁹⁷ Palecek S P, Schmidt C E, Lauffenburger D A, Horwitz A F. Integrin dynamics on the tail region of migrating fibroblasts. J Cell Sci. 1996 May; 109 (Pt 5):941-52.
²⁹⁸ Friedl P, Borgmann S, Brocker E B. Amoeboid leukocyte crawling through extracellular matrix: lessons from the Dictyostelium paradigm of cell movement. J Leukoc Biol. 2001 October; 70(4):491-509.
²⁹⁹ Holly S P, Larson M K, Parise L V. Multiple roles of integrins in cell motility. Exp Cell Res. 2000 Nov. 25; 261(1):69-74.
³⁰⁰ Bienvenu K, Harris N, Granger D N. Modulation of leukocyte migration in mesenteric interstitium. Am J Physiol. 1994 October; 267(4 Pt 2):H1573-7.
³⁰¹ Weber C, Springer T A. Interaction of very late antigen-4 with VCAM-1 supports transendothelial chemotaxis of monocytes by facilitating lateral migration. J, Immunol. 1998 Dec. 15; 161(12):6825-34.
³⁰² Weber C, Alon R, Moser B, Springer T A. Sequential regulation of alpha 4 beta 1 and alpha 5 beta I integrin avidity by CC chemokines in monocytes: implications for transendothelial chemotaxis. J. Cell Biol. 1996 August; 134(4):1063-73.
³⁰³ Chigaev A, Blenc A M, Braaten J V, Kumaraswamy N, Kepley C L, Andrews R P, Oliver J M, Edwards B S, Prossnitz E R, Larson R S, Sklar L A. Real time analysis of the affinity regulation of alpha 4-integrin. The physiologically activated receptor is intermediate in affinity between resting and Mn(2+) or antibody activation. J Biol. Chem. 2001 Dec. 28; 276(52):48670-8.
³⁰⁴ Cunningham F M, Wong E, Woollard P M, Greaves M W. The chemokinetic response of psoriatic and normal polymorphonuclear leukocytes to arachidonic acid lipoxygenase products. Arch Dermatol Res. 1986; 278(4):270-3.
³⁰⁵ Loike J D, Cao L, Budhu S, Hoffman S, Silvetsteini S C. Blockade of alpha 5 beta I integrins reverses the inhibitory effect of tenascin on chemotaxis of human monocytes and polymorphonuclear leukocytes through three-dimensional gels of extracellular matrix proteins. J. Immunol. 2001 Jun. 15; 166(12):7534-42.
³⁰⁶ Brady H R, Persson U, Ballermann B J, Brenner B M, Serhan C N. Leukotrienes stimulate neutrophil adhesion to mesangial cells: modulation with lipoxins. Am J. Physiol. 1990 November; 259(5 Pt 2):F809-15.
³⁰⁷ Lindstrom P, Lerner R, Palmblad J, Patarroyo M. Rapid adhesive responses of endothelial cells and of neutrophils induced by leukotriene B4 are mediated by leucocytic adhesion protein CD18. Scand J. Immunol. 1990 June; 31(6):737-44.
³⁰⁸ Seo S M, McIntire L V, Smith C W. Effects of IL-8, Gro-alpha, and LTB(4) on the adhesive kinetics of LFA-1 and Mac-1 on human neutrophils. Am J Physiol Cell Physiol. 2001 November; 281(5):C1568-78.
³⁰⁹ Nilsson E, Lindstrom P, Patarroyo M, Ringertz B. Lerner R, Rincon J, Palmblad J. Ethanol impairs certain aspects of neutrophil adhesion in vitro: comparisons with inhibition of expression of the CD18 antigen. J Infect Dis 1991 March; 163(3):591-7.
³¹⁰ Fretland D, Widomski D, Anglin C, Gaginella T. CD18 monoclonal antibody inhibits neutrophil diapedesis in the murine dermis induced by leukotriene B4 and 12(R)-hydroxyeicosatetraenoic acid. Eicosanoids. 1990; 3(3):171-4.
³¹¹ Sun R Z, Zhou D Y, Zheng M R, Yue T L. Chemotaxis of polymorphonuclear leukocytes towards LTB4 in patients with psoriasis in vitro. Chin Med J (Engl). 1990 July; 103(7):595-8.
³¹² Nordestgaard B G, Hjelms E, Stender S, Kjeldsen K. Different efflux pathways for high and low density lipoproteins from porcine aortic intima. Arteriosclerosis. 1990 May-June; 10(3):477-85.
³¹³ Pentikainen M O, Oorni K, Ala-Korpela M, Kovanen P T. Modified LDL—trigger of atherosclerosis and inflammation in the arterial intima. J Intern Med. 2000 March; 247(3):359-70.
³¹⁴ Bjornheden T, Bondjers G, Wiklund O. Direct assessment of lipoprotein outflow from in vivo-labeled arterial tissue as determined in an in vitro perfusion system. Arterioscler Thromb Vasc Biol. 1998 December; 18(12): 1927-33.
³¹⁵ Boren J, Olin K, Lee 1, Chait A, Wight T N, Innerarity TL. Identification of the principal proteoglycan-binding site in LDL. A single-point mutation in apo-B 100 severely affects proteoglycan interaction without affecting LDL receptor binding. J Clin Invest. 1998 Jun. 15; 101(12):2658-64.
³¹⁶ Nordestgaard B G, Tybjaerg-Hansen A, Lewis B. Influx in vivo of low density, intermediate density, and very low density lipoproteins into aortic intimas of genetically hyperlipidemic rabbits. Roles of plasma concentrations, extent of aortic lesion, and lipoprotein particle size as determinants. Arterioscler Thromb. 1992 January; 12(1):6-18.
³¹⁷ Schwenke D C. Comparison of aorta and pulmonary artery: IL LDL transport and metabolism correlate with susceptibility to atherosclerosis. Circ Res. 1997 September; 81(3):346-54.
³¹⁸ Kao C H, Chen J K, Yang V C. Ultrastructure and permeability of endothelial cells in branched regions of rat arteries. Atherosclerosis. 1994 January; 105(1):97-114.
³¹⁹ Kao C H, Chen J K, Kuo J S, Yang V C. Visualization of the transport pathways of low density lipoproteins across the endothelial cells in the branched regions of rat arteries. Atherosclerosis. 1995 July; 116(1):27-41.
³²⁰ Nordestgaard B G, Wootton R, Lewis B. Selective retention of VLDL, IDL, and LDL in the arterial intima of genetically hyperlipidemic rabbits in vivo. Molecular size as a determinant of fractional loss from the intima-inner media. Arterioscler Thromb Vasc Biol 1995 April; 15(4):534-42.
³²¹ Sanserson C M, Smith G L. Cell motility and cell morphology: how some viruses take control. 4 May 1999, http://www-ermm.cbcu.cam.ac.uk/99000629h.htm
³²² Kita T, Kume N, Minami M, Hayashida K, Murayama T, Sano H, Moriwaki H, Kataoka H, Nishi E, Horiuchi H, Arai H, Yokode M. Role of oxidized LDL in atherosclerosis. Ann N Y Acad. Sci. 2001 December; 947:199-205; discussion 205-6. Review.
³²³ Valente A J, Rozek M M, Sprague E A, Schwartz C J. Mechanisms in intimal monocyte-macrophage recruitment. A special role for monocyte chemotactic protein-1. Circulation. 1992 December; 86(6 Suppl):11120-5. Review.
³²⁴ Gerrity R G. The role of the monocyte in atherogenesis: II. Migration of foam cells from atherosclerotic lesions. Am J Pathol. 1981 May; 103(2):191-200.
³²⁵ Faggiotto A, Ross R, Harker L. Studies of hypercholesterolemia in the nonhuman primate. I. Changes that lead to fatty streak formation. Arteriosclerosis. 1984 July-August; 4(4):323-40.
³²⁶ Faggiotto A, Ross R. Studies of hypercholesterolemia in the nonhuman primate. II. Fatty streak conversion to fibrous plaque. Arteriosclerosis. 1984 July-August; 4(4)341-56.
³²⁷ Kling D, Holzschuh T, Betz E. Recruitment and dynamics of leukocytes in the formation of arterial intimal thickening—a comparative study with normo- and hypercholesterolemic rabbits. Atherosclerosis. 1993 June; 101(1):79-96.
³²⁸ Hynes R O. Integrins: versatility, modulation, and signaling in cell adhesion. Cell 1992 Apr. 3; 69(1): 11-25.
³²⁹ Duperray A, Languino L R, Plescia J, McDowall A, Hogg N, Craig A G, Berendt A R, Altieri D C. Molecular identification of a novel fibrinogen binding site on the first domain of ICAM-1 regulating leukocyte-endothelium bridging. J Biol. Chem. 1997 Jan. 3; 272(1):435-41.
³³⁰ D'Souza S E, Byers-Ward V J, Gardiner E E, Wang H, Sung S S. Identification of an active sequence within the first immunoglobulin domain of intercellular cell adhesion molecule-1 (ICAM-1) that interacts with fibrinogen. J Biol. Chem. 1996 Sep. 27; 271(39):24270-7.
³³¹ Languino L R, Duperray A, Joganic K J, Formaro M, Thornton G B, Altieri D C. Regulation of leukocyte-endothelium interaction and leukocyte transendothelial migration by intercellular adhesion molecule 1-fibrinogen recognition. Proc Natl Acad Sci USA. 1995 Feb. 28; 92(5):1505-9.
³³² Altieri D C, Duperray A, Plescia J, Thornton G B, Languino L R. Structural recognition of a novel fibrinogen gamma chain sequence (117-133) by intercellular adhesion molecule-1 mediates leukocyte-endothelium interaction. J Biol. Chem. 1995 Jan. 13; 270(2):696-9.
³³³ Shang X Z, Issekutz A C. Contribution of CD11a/CD18, CD11b/CD18, ICAM-1 (CD54) and -2 (CD102) to human monocyte migration through endothelium and connective tissue fibroblast barriers. Eur J. Immunol. 1998 June; 28(6): 1970-9.
³³⁴ Shang X Z, Lang B J, Issekutz A C. Adhesion molecule mechanisms mediating monocyte migration through synovial fibroblast and endothelium barriers: role for CD 11/CD18, very late antigen-4 (CD49d/CD29), very late antigen-5 (CD49e/CD29), and vascular cell adhesion molecule-1 (CD106). J. Immunol. 1998 Jan. 1; 160(1):467-74.
³³⁵ Meerschaert J, Furie M B. The adhesion molecules used by monocytes for migration across endothelium include CD11a/CD18, CD11b/CD138, and VLA-4 on monocytes and ICAM-1, VCAM-1, and other ligands on endothelium. J. Immunol. 1995 Apr. 15; 154(8):4099-112.
³³⁶ Meerschaert J, Furie M B. Monocytes use either CD11/CD18 or VLA-4 to migrate across human endothelium in vitro. J. Immunol. 1994 Feb. 15; 152(4):1915-26.
³³⁷ Chuluyan H E, Issekutz A C. VLA-4 integrin can mediate CD11/CD18-independent transendothelial migration of human monocytes. J Clin Invest. 1993 December; 92(6):2768-77.
³³⁸ Kavanaugh A F, Lightfoot E, Lipsky P E, Oppenheimer-Marks N. Role of CD11/CD18 in adhesion and transendothelial migration of T-cells. Analysis utilizing CD18-deficient T-cell clones. J Immunol. 1991Jun. 15; 146(12):4149-56.
³³⁹ Fernandez-Segura E, Garcia J M, Campos A. Topographic distribution of CD18 integrin on human neutrophils as related to shape changes and movement induced by chemotactic peptide and phorbol esters. Cell Immunol. 1996 Jul. 10; 171(1):120-5.
³⁴⁰ Carson S D, Pirruccello S J. Immunofluorescent studies of tissue factor on U87MG cells: evidence for non-uniform distribution. Blood Coagul Fibrinolysis. 1993 December; 4(6):911-20.
³⁴¹ Lewis J C, Bennett-Cain A L, DeMars C S, Doellgast G J, Grant K W, Jones N L, Gupta M. Procoagulant activity after exposure of monocyte-derived macrophages to minimally oxidized low density lipoprotein. Co-localization of tissue factor antigen and nascent fibrin fibers at the cell surface. Am J Pathol. 1995 October; 147(4):1029-40.
³⁴² Muller M, Albrecht S, Golfert F, Hofer A, Funk R H, Magdolen V, Flossel C, Luther T. Localization of tissue factor in actin-filament-rich membrane areas of epithelial cells. Exp Cell Res. 1999 Apr. 10; 248(1):136-47.
³⁴³ Ott I, Fischer E G, Miyagi Y, Mueller B M, Ruf W. A role for tissue factor in cell adhesion and migration mediated by interaction with actin-binding protein 280. J. Cell Biol. 1998 Mar. 9; 146(5):1241-53.
³⁴⁴ Cunningham C C, Gorlin J B, Kwiatkowski D J, Hartwig J H, Janmey P A, Byers H R, Stossel T P. Actin-binding protein requirement for cortical stability and efficient locomotion. Science. 1992 Jan. 17; 255(5042):325—7.
³⁴⁵ Randolph G J, Luther T, Albrecht S, Magdolen V, Muller W A. Role of tissue factor in adhesion of mononuclear phagocytes to and trafficking through endothelium in vitro. Blood. 1998 Dec. 1; 92(11):4167-77.
³⁴⁶ Fan S T, Mackman N, Cui M Z, Edgington T S. Integrin regulation of an inflammatory effector gene. Direct induction of the tissue factor promoter by engagement of beta I or alpha 4 integrin chains. J. Immunol. 1995 Apr 1; 154(7):3266-74.
³⁴⁷ McGilvray I D, Lu Z, Bitar R, Dackiw A P, Davreux C J, Rotstein O D. VLA-4 integrin cross-linking on human monocytic THP-1 cells induces tissue factor expression by a mechanism involving mitogen-activated protein kinase. J Biol. Chem. 1997 Apr 11; 272(15):10287-94.
³⁴⁸ McGilvray I D, Lu Z, Wei A C, Rotstein O D. MAP-kinase dependent induction of monocytic procoagulant activity by beta2-integrins. J Surg Res. 1998 December; 80(2):272-9.
³⁴⁹ Fan S T, Edgington T S. Coupling of the adhesive receptor CD11b/CD18 to functional enhancement of effector macrophage tissue factor response. J Clin Invest. 1991 January; 87(1):50-7.
³⁵⁰ Marx N, Neumann F J, Zohlnhofer D, Dickfeld T, Fischer A, Heimerl S, Schomig A. Enhancement of monocyte procoagulant activity by adhesion on vascular smooth muscle cells and intercellular adhesion molecule-1-transfected Chinese hamster ovary cells. Circulation. 1998 Sep. 1; 98(9):906-11.
³⁵¹ Lund T. Osterud B. Fibrinogen increases lipopolysaccharide-induced tumor necrosis factor-alpha and interleukin-8 release, and enhances tissue factor activity in monocytes in a modified whole blood system. Blood Coagul Fibrinolysis. 2001 December; 12(8):667-75.
³⁵² Jones P L, Cowan K N, Rabinovitch M. Tenascin-C, proliferation and subendothelial fibronectin in progressive pulmonary vascular disease. Am J Pathol. 1997 April; 150(4):1349-60.
³⁵³ Tanouchi J, Uematsu M, Kitabatake A, Masuyama T, Ito H, Doi Y, Inoue M, Kamada T. Sequential appearance of fibronectin, collagen and elastin during fatty streak initiation and maturation in hypercholesterolemic fat-fed rabbits. Jpn Circ J. 1992 July; 56(7):649-56.
³⁵⁴ Shekhonin B V, Domogatsky S P, Idelson G L, Koteliansky V E, Rukosuev V S. Relative distribution of fibronectin and type I, III, IV, V collagens in normal and atherosclerotic intima of human arteries. Atherosclerosis. 1987 September; 67(1):9-16.
³⁵⁵ Lou X J, Boonmark N W, Horrigan F T, Degen J L, Lawn R M. Fibrinogen deficiency decreases vascular accumulation of apolipoprotein(a) and development of atherosclerosis in apolipoprotein(a) transgenic mice. Proc Natl Acad Sci USA. 1998 Oct. 13; 95(21):12591-5.
³⁵⁶ Xiao Q, Danton M J, Witte D P, Kowala M C, Valentine M T, Degen J L. Fibrinogen deficiency is compatible with the development of atherosclerosis in mice. J Clin Invest. 1998 Mar. 1; 101(5):1184-94.
³⁵⁷ O'Brien K D, Allen M D, McDonald T O, Chait A, Harlan J M, Fishbein D, McCarty J, Ferguson M, Hudkins K, Benjamin C D, et al. Vascular cell adhesion molecule-1 is expressed in human coronary atherosclerotic plaques. Implications for the mode of progression of advanced coronary atherosclerosis. J Clin Invest. 1993 August; 92(2):945-51.
³⁵⁸ Li H, Cybulsky M I, Gimbrone M A Jr, Libby P. Inducible expression of vascular cell adhesion molecule-1 by vascular smooth muscle cells in vitro and within rabbit atheroma. Am J Pathol. 1993 December; 143(6):1551-9.
³⁵⁹ Thibault G, Lacombe M J, Schnapp L M, Lacasse A, Bouzeghrane F, Lapalme G. Upregulation of alpha(8)beta(1)-integrin in cardiac fibroblast by angiotensin II and transforming growth factor-beta1. Am J Physiol Cell Physiol. 2001 November; 281(5):C1457-67.
³⁶⁰ Sixt M, Hallmann R, Wendler O, Scharffetter-Kochanek K, Sorokin L M. Cell adhesion and migration properties of beta 2-integrin negative polymorphonuclear granulocytes on defined extracellular matrix molecules. Relevance for leukocyte extravasation. J Biol. Chem. 2001 Jun. 1; 276(22):18878-87.
³⁶¹ Tall A R, Costet P, Wang N. Regulation and mechanisms of macrophage cholesterol efflux. J Clin Invest. 2002 October; 110(7):899-904. Review.
³⁶² von Eckardstein A, Nofer J R, Assmann G. High density lipoproteins and arteriosclerosis. Role of cholesterol efflux and reverse cholesterol transport. Arterioscler Thromb Vasc Biol. 2001 January; 21(1):13-27. Review.
³⁶³ Rothblat G H, de la Llera-Moya M, Atger V, Kellner-Weibel G, Williams D L, Phillips M C. Cell cholesterol efflux: integration of old and new observations provides new insights. J Lipid Res. 1999 May; 40(5):781-96. Review.
³⁶⁴ Phillips M C, Gillotte K L, Haynes M P, Johnson W J, Lund-Katz S, Rothblat G H. Mechanisms of high density lipoprotein-mediated efflux of cholesterol from cell plasma membranes. Atherosclerosis. 1998 April; 137 Suppl:S13-7. Review.
³⁶⁵ Yokoyama S. Apolipoprotein-mediated cellular cholesterol efflux. Biochim Biophys Acta. 1998 May 20; 1392(1):1-15. Review.
³⁶⁶ Rong J X, Li J, Reis E D, Choudhury R P, Dansky H M, Elmalem V I, Fallon J T, Breslow J L, Fisher E A. Elevating high-density lipoprotein cholesterol in apolipoprotein E-deficient mice remodels advanced atherosclerotic lesions by decreasing macrophage and increasing smooth muscle cell content. Circulation. 2001 Nov. 13; 104(20):2447-52.
³⁶⁷ Ishiguro H, Yoshida H, Major A S, Zhu T, Babaev V R, Linton M F, Fazio S. Retrovirus-mediated expression of apolipoprotein A-I in the macrophage protects against atherosclerosis in vivo. J Biol. Chem. 2001 Sep. 28; 276(39):36742-8.
³⁶⁸ Major A S, Dove D E, Ishiguro H, Su Y R, Brown A M, Liu L, Carter K J, Linton M F, Fazio S. Increased cholesterol efflux in apolipoprotein AI (ApoAI)-producing macrophages as a mechanism for decreased atherosclerosis in ApoAI((−/−)) mice. Arterioscler Thromb Vasc Biol. 2001 November; 21(11):1790-5.
³⁶⁹ Duverger N, Kruth H, Emmanuel F, Caillaud J M, Viglietta C, Castro G, Tailleux A, Fievet C, Fruchart J C, Houdebine L M, Denefle P. Inhibition of atherosclerosis development in cholesterol-fed human apolipoprotein A-I-transgenic rabbits. Circulation. 1996 Aug. 15; 94(4):713-7.
³⁷⁰ Plump A S, Scott C J, Breslow J L. Human apolipoprotein A-I gene expression increases high density lipoprotein and suppresses atherosclerosis in the apolipoprotein E-deficient mouse. Proc Natl Acad Sci USA. 1994 Sep. 27; 91(20):9607-11.
³⁷¹ Shah P K, Yano J, Reyes O, Chyu K Y, Kaul S, Bisgaier C L, Drake S, Cercek B. High-dose recombinant apolipoprotein A-I(milano) mobilizes tissue cholesterol and rapidly decreases plaque lipid and macrophage content in apolipoprotein e-deficient mice. Potential implications for acute plaque stabilization. Circulation. 2001 Jun. 26; 103(25):3047-50.
³⁷² Dansky H M, Charlton S A, Barlow C B, Tamminen M, Smith J D, Frank J S, Breslow J L. Apo A-I inhibits foam cell formation in Apo E-deficient mice after monocyte adherence to endothelium. J Clin Invest. 1999 July; 104(1):31-9.
³⁷³ Therond P, Abella A, Laurent D, Couturier M, Chalas J, Legrand A, Lindenbaum A. In vitro study of the cytotoxicity of isolated oxidized lipid low-density lipoproteins fractions in human endothelial cells: relation with the glutathione status and cell morphology. Free Radic Biol Med. 2000 Feb. 15; 28(4):585-96.
³⁷⁴ Lizard G, Gueldry S, Sordet O, Monier S, Athias A, Miguet C, Bessede G, Lemaire S, Solary E, Gambert P. Glutathione is implied in the control of 7-ketocholesterol-induced apoptosis, which is associated with radical oxygen species production. FASEB J. 1998 December; 12(15):1651-63.
³⁷⁵ Crutchley D J, Que B G. Copper-induced tissue factor expression in human monocytic THP-1 cells and its inhibition by antioxidants. Circulation. 1995 Jul. 15; 92(2):238-43.
³⁷⁶ Caspar-Bauguil S, Tkaczuk J, Haure M J, Durand M, Alcouffe J, Thomsen M, Salvayre R, Benoist H. Mildly oxidized low-density lipoproteins decrease early production of interleukin 2 and nuclear factor kappaB binding to DNA in activated T-lymphocytes. Biochem J. 1999 Jan. 15; 337 (Pt 2):269-74.
³⁷⁷ Matsumura T, Sakai M, Matsuda K, Furukawa N, Kaneko K, Shichiri M. Cis-acting DNA elements of mouse granulocyte/macrophage colony-stimulating factor gene responsive to oxidized low density lipoprotein. J Biol. Chem. 1999 Dec. 31; 274(53):37665-72.
³⁷⁸ Hamilton T A, Major J A, Armstrong D, Tebo J M. Oxidized LDL modulates activation of NFkappaB in mononuclear phagocytes by altering the degradation if IkappaBs. J Leukoc Biol. 1998 November; 64(5):667-74.
³⁷⁹ Schackelford R E, Misra U K, Florine-Casteel K, Thai S F, Pizzo S V, Adams D O. Oxidized low density lipoprotein suppresses activation of NF kappa B in macrophages via a pertussis toxin-sensitive signaling mechanism. J Biol. Chem. 1995 Feb. 24; 270(8):3475-8.
³⁸⁰ Ohlsson B G, Englund M C, Karlsson A L, Knutsen E, Erixon C, Skribeck H, Liu Y, Bondjers G, Wiklund O. Oxidized low density lipoprotein inhibits lipopolysaccharide-induced binding of nuclear factor-kappaB to DNA and the subsequent expression of tumor necrosis factor-alpha and interleukin-1 beta in macrophages. J Clin Invest 1996 Jul. 1; 98(1):78-89.
³⁸¹ Ares M P, Kallin B, Eriksson P, Nilsson J. Oxidized LDL induces transcription factor activator protein-I but inhibits activation of nuclear factor-kappa B in human vascular smooth muscle cells. Arterioscler Thromb Vasc Biol. 1995 October; 15(10):1584-90.
³⁸² Yan S D, Schmidt A M, Anderson G M, Zhang J, Brett J, Zou Y S, Pinsky D, Stern D. Enhanced cellular oxidant stress by the interaction of advanced glycation end products with their receptors/binding proteins. J Biol. Chem. 1994 Apr. 1; 269(13):9889-97.
³⁸³ Khechai F, Ollivier V, Bridey F, Amar M, Hakim J, de Prost D. Effect of advanced glycation end product-modified albumin on tissue factor expression by monocytes. Role of oxidant stress and protein tyrosine kinase activation. Arterioscler Thromb Vasc Biol. 1997 November; 17(11):2885-90.
³⁸⁴ Brisseau G F, Dackiw A P, Cheung P Y, Christie N, Rotstein O D. Posttranscriptional regulation of macrophage tissue factor expression by antioxidants. Blood 1995 Feb. 15; 85(4):1025-35.
³⁸⁵ Ichikawa K, Yoshinari M, Iwase M, Wakisaka M, Doi Y, Iino K, Yamamoto M, Fujishima M. Advanced glycosylation end products induced tissue factor expression in human monocyte-like U937 cells and increased tissue factor expression in monocytes from diabetic patients. Atherosclerosis. 1998 February; 136(2):281-7.
³⁸⁶ Lesnik P, Rouis M, Skarlatos S. Kruth H S, Chapman M J. Uptake of exogenous free cholesterol induces upregulation of tissue factor expression in human monocyte-derived macrophages. Proc Natl Acad Sci USA. 1992Nov. 1; 89(21):10370-4.
³⁸⁷ Ohsawa M, Koyama T, Yamamoto K, Hirosawa S, Kamei S, Kamiyania R. 1alpha,25-dihydroxyvitamin D(3) and its potent synthetic analogs downregulate tissue factor and upregulate thrombomodulin expression in monocytic cells, counteracting the effects of tumor necrosis factor and oxidized LDL. Circulation. 2000 Dec. 5; 102(23):2867-72.
³⁸⁸ Cui M Z, Penn M S, Chisolm G M. Native and oxidized low density lipoprotein induction of tissue factor gene expression in smooth muscle cells is mediated by both Egr-1 and Sp1. J Biol. Chem. 1999 Nov. 12; 274(46):32795-802.
³⁸⁹ Penn M S, Cui M Z, Winokur A L, Bethea J, Hamilton T A, DiCorleto P E, Chisolm G M. Smooth muscle cell surface tissue factor pathway activation by oxidized low density lipoprotein requires cellular lipid peroxidation. Blood. 2000 Nov. 1; 96(9):3056-63.
³⁹⁰ Penn M S, Patel C V, Cui M Z, DiCorleto P E, Chisolm G M. LDL increases inactive tissue factor on vascular smooth muscle cell surfaces: hydrogen peroxide activates latent cell surface tissue factor. Circulation. 1999 Apr. 6; 99(13):1753-9.
³⁹¹ Fei H, Berliner J A, Parhami F, Drake T A. Regulation of endothelial cell tissue factor expression by minimally oxidized LDL and lipopolysaccharide. Arterioscler Thromb. 1993 November; 13(11):1711-7.
³⁹² Verhamme P, Quarck R, Hao H, Knaapen M, Dymarkowski S, Bermar H, Van Cleemput J, Janssens S, Vermylen J, Gabbiani G, Kockx M, Holvoet P. Dietary cholesterol withdrawal decreases vascular inflammation and induces coronary plaque stabilization in miniature pigs. Cardiovasc Res. 2002 October; 56(1): 135-44.
³⁹³ Okura Y, Brink M, Itabe H, Scheidegger K J, Kalangos A, Delafontaine P. Oxidized low-density lipoprotein is associated with apoptosis of vascular smooth muscle cells in human atherosclerotic plaques. Circulation. 2000 Nov. 28; 102(22):2680-6.
³⁹⁴ Trach C C, Wulfroth P M, Severs N J, Robenek H. Influence of native and modified lipoproteins on migration of mouse peritoneal macrophages and the effect of the antioxidants vitamin E and Probucol. Eur J. Cell Biol. 1996 October; 71(2):199-205.
³⁹⁵ Pataki M, Lusztig G, Robenek H. Endocytosis of oxidized LDL and reversibility of migration inhibition in macrophage-derived foam cells in vitro. A mechanism for atherosclerosis regression? Arterioscler Thromb. 1992 August; 12(8):936-44.
³⁹⁶ Wissler R W, Vesselinovitch D. Can atherosclerotic plaques regress? Anatomic and biochemical evidence from nonhuman animal models. Am J Cardiol. 1990 Mar. 20; 65(12):33F40F.
³⁹⁷ Dudrick S J. Regression of atherosclerosis by the intravenous infusion of specific biochemical nutrient substrates in animals and humans. Ann Surg. 1987 September; 206(3):296-315.
³⁹⁸ Tucker C F, Catsulis C, Strong J P, Eggen D A. Regression of early cholesterol-induced aortic lesions in rhesus monkeys. Am J Pathol. 1971 December; 65(3):493-514.
³⁹⁹ Skalen K, Gustafsson M, Rydberg E K, Hulten L M, Wiklund O, Innerarity T L, Boren J. Subendothelial retention of atherogenic lipoproteins in early atherosclerosis. Nature. 2002 Jun. 13; 417(6890):750-4.
⁴⁰⁰ Proctor S D, Vine D F, Mamo J C. Arterial retention of apolipoprotein B(48)- and B(100)-containing lipoproteins in atherogenesis. Curr Opin Lipidol. 2002 October; 13(5):461-70.
⁴⁰¹ Williams K J, Tabas I. The response-to-retention hypothesis of atherogenesis reinforced. Curr Opin Lipidol. 1998 October; 9(5):471-4. Review.
⁴⁰² Malek A M, Alper S L, Izumo S. Hemodynamic shear stress and its role in atherosclerosis. JAMA. 1999 Dec. 1; 282(21):2035-42.
⁴⁰³ Utermann G. The mysteries of lipoprotein(a). Science. 1989 Nov. 17; 246(4932):904-10. Review.
⁴⁰⁴ Fan Z, Larson P J, Bognacki J, Raghunath P N, Tomaszewski J E, Kuo A, Canziani G, Chaiken I, Cines D B, Higazi A A. Tissue factor regulates plasminogen binding and activation. Blood. 1998 Mar. 15; 91(6):1987-98.
⁴⁰⁵ Moser T L, Enghild J J, Pizzo S V, Stack M S. The extracellular matrix proteins laminin and fibronectin contain binding domains for human plasminogen and tissue plasminogen activator. J Biol. Chem. 1993 Sep. 5; 268(25): 18917-23.
⁴⁰⁶ Salonen E M, Saksela O, Vartio T, Vaheri A, Nielsen L S, Zeuthen J. Plasminogen and tissue-type plasminogen activator bind to immobilized fibronectin. J Biol. Chem. 1985 Oct. 5; 260(22):12302-7.
⁴⁰⁷ Bendixen E, Borth W, Harpel P C. Transglutaminases catalyze cross-linking of plasminogen to fibronectin and human endothelial cells. J Biol. Chem. 1993 Oct. 15; 268(29):21962-7.
⁴⁰⁸ Xia J, May L F, Koschinsky M L. Characterization of the basis of lipoprotein [a]lysine-binding heterogeneity. J Lipid Res. 2000 October; 41(10):1578-84.
⁴⁰⁹ Kochl S, Fresser F, Lobentanz E, Baier G, Utermann G. Novel interaction of apolipoprotein(a) with beta-2 glycoprotein I mediated by the kringle IV domain. Blood. 1997 Aug. 15; 90(4):1482-9.
⁴¹⁰ Salonen E M, Jauhiainen M, Zardi L, Vaheri A, Ehnholm C. Lipoprotein(a) binds to fibronectin and has serine proteinase activity capable of cleaving it. EMBO J. 1989 Dec. 20; 8(13):403540.
⁴¹¹ Ehnholm C, Jauhiainen M, Metso J. Interaction of lipoprotein(a) with fibronectin and its potential role in atherogenesis. Eur Heart J. 1990 August; 11 Suppl E:190-5. Review.
⁴¹² van der Hoek Y Y, Sangrar W, Cote G P, Kastelein J J, Koschinsky M L. Binding of recombinant apolipoprotein(a) to extracellular matrix proteins. Arterioscler Thromb. 1994 November; 14(11):1792-8.
⁴¹³ Pekelharing H L, Kleinveld H A, Duif P F, Bouma B N, van Rijn H J. Effect of lipoprotein(a) and LDL on plasminogen binding to extracellular matrix and on matrix-dependent plasminogen activation by tissue plasminogen activator. Thromb Haemost. 1996 March; 75(3):497-502.
⁴¹⁴ Kark J D, Sandholzer C, Friedlander Y, Utermann G. Plasma Lp(a), apolipoprotein(a) isoforms and acute myocardial infarction in men and women: a case-control study in the Jerusalem population. Atherosclerosis. 1993 Jan. 25; 98(2):139-51.
⁴¹⁵ Wild S H, Fortmann S P, Marcovina S M. A prospective case-control study of lipoprotein(a) levels and apo(a) size and risk of coronary heart disease in Stanford Five-City Project participants. Arterioscler Thromb Vasc Biol. 1997 February; 17(2):23945.
⁴¹⁶ Rhoads G G, Dahlen G, Berg K, Morton N E, Dannenberg A L. Lp(a) lipoprotein as a risk factor for myocardial infarction. JAMA. 1986 Nov. 14; 256(18):2540-4.
⁴¹⁷ Kronenberg F, Kronenberg M F, Kiechl S, Trenkwalder E, Santer P, Oberhollenzer F, Egger G, Utermann G, Willeit J. Role of lipoprotein(a) and apolipoprotein(a) phenotype in atherogenesis: prospective results from the Bruneck study. Circulation. 1999 Sep. 14; 100(11):1154-60.
⁴¹⁸ Thillet J, Doucet C, Chapman J, Herbeth B, Cohen D, Faure-Delanef L. Elevated lipoprotein(a) levels and small apo(a) isoforms are compatible with longevity: evidence from a large population of French centenarians. Atherosclerosis. 1998 February; 136(2):389-94.
⁴¹⁹ Baggio G, Donazzan S, Monti D, Mari D, Martini S, Gabelli C, Dalla Vestra M, Previato L, Guido M, Pigozzo S, Cortella I, Crepaldi G, Franceschi C. Lipoprotein(a) and lipoprotein profile in healthy centenarians: a reappraisal of vascular risk factors. FASEB J. 1998 April; 12(6):433-7.
⁴²⁰ DePrince K, McGarvey S T, McAllister A E, Bausserman L, Aston C E, Ferrell R E, Kamboh M I. Genetic effect of two APOA repeat polymorphisms (kringle 4 and pentanucleotide repeats) on plasma Lp(a) levels in American Samoans. Hum Biol. 2001 February; 73(1):91-104.
⁴²¹ Chiu L, Hamman R F, Kamboh M I. Apolipoprotein A polymorphisms and plasma lipoprotein(a) concentrations in non-Hispanic Whites and Hispanics. Hum Biol. 2000 October; 72(5):821-35.
⁴²² Valenti K, Aveynier E, Leaute S, Laporte F, Hadjian A J. Contribution of apolipoprotein(a) size, pentanucleotide TTTTA repeat and C/T(+93) polymorphisms of the apo(a) gene to regulation of lipoprotein(a) plasma levels in a population of young European Caucasians. Atherosclerosis. 1999 Nov. 1; 147(1):17-24.
⁴²³ Gaw A, Brown E A, Ford I. Impact of apo(a) length polymorphism and the control of plasma Lp(a) concentrations: evidence for a threshold effect. Arterioscler Thromb Vasc Biol. 1998 December; 18(12):1870-6.
⁴²⁴ Valenti K, Aveynier E, Laporte F, Hadjian A J. Evaluation of the genotyping and phenotyping approaches in the investigation of apolipoprotein (a) size polymorphism. Clin Chim Acta. 1997 Jul. 25; 263(2):249-60.
⁴²⁵ de la Pena-Diaz A, Izaguirre-Avila R, Angles-Cano E. Lipoprotein Lp(a) and atherothrombotic disease. Arch Med Res 2000 July-August; 31(4):353-9.
⁴²⁶ Pati U, Pati N. Lipoprotein(a), atherosclerosis, and apolipoprotein(a) gene polymorphism. Mol Genet Metab. 2000 September-October; 71(1-2):87-92.
⁴²⁷ Beisiegel U, Niendorf A, Wolf K, Reblin T, Rath M. Lipoprotein(a) in the arterial wall. Eur Heart J. 1990 August; 11 Suppl E:174-83.
⁴²⁸ Rath M, Niendorf A, Reblin T, Dietel M, Krebber H J, Beisiegel U. Detection and quantification of lipoprotein(a) in the arterial wall of 107 coronary bypass patients. Arteriosclerosis. 1989 September-October; 9(5):579-92.
⁴²⁹ Ichikawa T, Unoki H. Sun H, Shimoyamada H, Marcovina S, Shikama H, Watanabe T, Fan J. Lipoprotein(a) promotes smooth muscle cell proliferation and dedifferentiation in atherosclerotic lesions of human apo(a) transgenic rabbits. Am J Pathol. 2002 January; 160(1):227-36.
⁴³⁰ Fan J, Shimoyamada H, Sun H, Marcovina S, Honda K, Watanabe T. Transgenic rabbits expressing human apolipoprotein(a) develop more extensive atherosclerotic lesions in response to a cholesterol-rich diet. Arterioscler Thromb Vasc Biol. 2001 January; 21(1):88-94.
⁴³¹ Dangas G, Mehran R, Harpel P C, Sharma S K, Marcovina S M, Dube G, Ambrose J A, Fallon J T. Lipoprotein(a) and inflammation in human coronary atheroma: association with the severity of clinical presentation. J Am Coll Cardiol. 1998 December; 32(7):2035-42,
⁴³² Reblin T, Meyer N, Labeur C, Henne-Bruns D, Beisiegel U. Extraction of lipoprotein(a), apo B, and apo E from fresh human arterial wall and atherosclerotic plaques. Atherosclerosis. 1995 March; 113(2):179-88.
⁴³³ Hoff H F, O'Neil J, Yashiro A. Partial characterization of lipoproteins containing apo[a] in human atherosclerotic lesions. J Lipid Res. 1993 May; 34(5):789-98.
⁴³⁴ Kusumi Y, Scanu A M, McGill H C, Wissler R W. Atherosclerosis in a rhesus monkey with genetic hypercholesterolemia and elevated plasma Lp(a). Atherosclerosis 1993 March; 99(2):165-74.
⁴³⁵ Pepin J M, O'Neil J A, Hoff H F. Quantification of apo[a] and apoB in human atherosclerotic lesions. J Lipid Res. 1991 February; 32(2):317-27.
⁴³⁶ Boonmark N W, Lou X J, Yang Z J, Schwartz K, Zhang J L, Rubin E M, Lawn R M. Modification of apolipoprotein(a) lysine binding site decreases atherosclerosis in transgenic mice. J Clin Invest. 1997 Aug. 1; 100(3):558-64.
⁴³⁷ Lawn R M, Wade D P, Hammer R E, Chiesa G, Verstuyft J G, Rubin E M. Atherogenesis in transgenic mice expressing human apolipoprotein(a). Nature. 1992 Dec. 17; 360(6405):670-2.
⁴³⁸ Griffioen A W, Molema G. Angiogenesis: potentials for pharmacologic intervention in the treatment of cancer, cardiovascular diseases, and chronic inflammation. Pharmacol Rev. 2000 June; 52(2):237-68. Review.
⁴³⁹ Reijerkerk A, Voest E E, Gebbink M F. No grip, no growth: the conceptual basis of excessive proteolysisin the treatment of cancer. Eur J Cancer. 2000 August; 36(13 Spec No):1695-705. Review.
⁴⁴⁰ O'Reilly M S, Holmgren L, Shing Y, Chen C, Rosenthal R A, Moses M, Lane W S, Cao Y, Sage E H, Folkman J. Angiostatin: a novel angiogenesis inhibitor that mediates the suppression of metastases by a Lewis lung carcinoma. Cell. 1994 Oct 21; 79(2):315-28.
⁴⁴¹ Cao Y, Veitonmaki N, Keough K, Cheng H, Lee L S, Zurakowski D. Elevated levels of urine angiostatin and plasminogen/plasmin in cancer patients. Int J Mol Med. 2000 May; 5(5):547-51.
⁴⁴² Ribatti D, Vacca A, Giacchetta F, Cesaretti S, Anichini M, Roncali L, Damacco F. Lipoprotein (a) induces angiogenesis on the chick embryo chorioallantoic membrane. Eur J Clin Invest. 1998 July; 28(7):533-7.
⁴⁴³ Bdeir K, Cane W, Canziani G, Chaiken I, Weisel J, Koschinsky M L, Lawn R M, Bannerman P G, Sachais B S, Kuo A, Hancock M A, Tomaszewski J, Raghunath P N, Ganz T, Higazi A A, Cines D B. Defensin promotes the binding of lipoprotein(a) to vascular matrix. Blood. 1999 Sep. 15; 94(6):2007-19.
⁴⁴⁴ Higazi A A, Lavi E, Bdeir K, Ulrich A M, Jamieson D G, Rader D J, Usher D C, Kane W, Ganz T, Cines D B. Defensin stimulates the binding of lipoprotein (a) to human vascular endothelial and smooth muscle cells. Blood. 1997 Jun. 15; 89(12):4290-8.
⁴⁴⁵ Yano Y, Shimokawa K, Okada Y, Noma A. Immunolocalization of lipoprotein(a) in wounded tissues. J Histochem Cytochem. 1997 April; 45(4):559-68.
⁴⁴⁶ Ryan M J, Emig L L, Hicks G W, Ramharack R, Spahr M A, Kreick J S, Brammer D W, Chien A J, Keiser J A. Localization of lipoprotein(a) in a monkey model of rapid neointimal growth. Arterioscler Thromb Vasc Biol. 1997 January; 17(1):181-7.
⁴⁴⁷ Ryan M J, Emig L L, Hicks G W, Ramharack R, Brammer D W, Gordon D, Auerbach B J, Keiser J A. Influence of lipoprotein(a) plasma concentration on neointimal growth in a monkey model of vascular injury. Atherosclerosis. 1998 July; 139(1):137-45.
⁴⁴⁸ Nielsen L B, Stender S, Kjeldsen K, Nordestgaard B G. Specific accumulation of lipoprotein(a) in balloon-injured rabbit aorta in vivo. Circ Res. 1996 April; 78(4):615-26.
⁴⁴⁹ Maeda S, Abe A, Seishima M, Makino K, Noma A, Kawade M. Transient changes of serum lipoprotein(a) as an acute phase protein. Atherosclerosis. 1989 August; 78(2-3):145-50.
⁴⁵⁰ Noma A, Abe A, Maeda S, Seishima M, Makino K, Yano Y, Shimokawa K. Lp(a): an acute-phase reactant? Chem Phys Lipids. 1994 January; 67-68:411-7.
⁴⁵¹ Min W K, Lee J O, Huh J W. Relation between lipoprotein(a) concentrations in patients with acute-phase response and risk analysis for coronary heart disease. Clin Chem. 1997 October; 43(10):1891-5.
⁴⁵² Kronenberg F, Auinger M, Trenkwalder E, Irsigler K, Utermann G, Dieplinger H. Is apolipoprotein(a) a susceptibility gene for type I diabetes mellitus and related to long-term survival? Diabetologia. 1999 August; 42(8): 1021-7.
⁴⁵³ Wahn F, Daniel V, Kronenberg F, Opelz G, Michalk D V, Querfeld U. Impact of apolipoprotein(a) phenotypes on long-term renal transplant survival. J Am Soc Nephrol. 2001 May; 12(5):1052-8.
⁴⁵⁴ Witzenbichler B, Kureishi Y, Luo Z, Le Roux A, Branellec D, Walsh K. Regulation of smooth muscle cell migration and integrin expression by the Gax transcription factor. J Clin Invest. 1999 November; 104(10):1469-80.
⁴⁵⁵ Lippi G, Guidi G. Lipoprotein(a): from ancestral benefit to modern pathogen? QJM. 2000 February; 93(2):75-84. Review.
⁴⁵⁶ Kostner K M, Kostner G M. Lipoprotein(a): still an enigma? Curr Opin Lipidol. 2002 August; 13(4):391-6.
⁴⁵⁷ Scanu A M. Atherothrombogenicity of lipoprotein(a): the debate. Am J Cardiol. 1998 Nov. 5; 82(9A):26Q-33Q. Review.
⁴⁵⁸ Hobbs H H, White A L. Lipoprotein(a): intrigues and insights. Curr Opin Lipidol. 1999 June; 10(3):225-36. Review.
⁴⁵⁹ Goldstein M R. Lipoprotein(a): friend or foe? Am J Cardiol. 1995 Feb. 1; 75(4):319.
⁴⁶⁰ Mac Neil S, Wagner M, Rennie I G. Tamoxifen inhibition of ocular melanoma cell attachment to matrix proteins. Pigment cell Res. 1994 August; 7(4):222-6.
⁴⁶¹ Mac Neil S, Wagner M, Rennie I G. Investigation of the role of signal transduction in attachment of ocular melanoma cells to matrix proteins: inhibition of attachment by calmodulin antagonists including tamoxifen. Clin Exp Metastasis. 1994 November; 12(6):375-84.
⁴⁶² Millon R, Nicora F, Muller D, Eber M, Klein-Soyer C, Abecassis J. Modulation of human breast cancer cell adhesion by estrogens and antiestrogens. Clin Exp Metastasis. 1989 July-August; 7(4):405-15.
⁴⁶³ Wagner M, Benson M T, Rennie I G, MacNeil S. Effects of pharmacological modulation of intracellular signalling systems on retinal pigment epithelial cell attachment to extracellular matrix proteins. Curr Eye Res. 1995 May; 14(5):373-84.
⁴⁶⁴ Mohindroo A, Ahluwalia P. Effect of trifluoperazine on certain arterial wall lipid-metabolizing enzymes inducing atherosclerosis in rhesus monkeys. Lipids. 1997 August; 32(8):867-72.
⁴⁶⁵ Mohindroo A, Kukreja R S, Kaul D. Preventive effect of trifluoperazine on atherosclerosis induced by cholesterol & adrenaline in rabbits. Indian J Med Res. 1989 June; 90:215-9.
⁴⁶⁶ Kaul D, Kukreja R S, Sapru R P. Preventive effect of trifluoperazine on cholesterol-induced atherosclerosis in rabbits. Indian J Med Res. 1987 November; 86:678-84.
⁴⁶⁷ Kaul D, Kukreja R S. Atherogenesis. Preventive action of trifluoperazine. Atherosclerosis. 1987 April; 64(2-3):211-4.
⁴⁶⁸ McGilvray I D, Tsai V, Marshall J C, Dackiw A P, Rotstein O D. Monocyte adhesion and transmigration induce tissue factor expression: role of the mitogen-activated protein kinases. Shock. 2002 July; 18(1):51-7.
⁴⁶⁹ Probstmeier R, Pesheva P. Tenascin-C inhibits beta1 integrin-dependent cell adhesion and neurite outgrowth on fibronectin by a disialoganglioside-mediated signaling mechanism. Glycobiology. 1999 February; 9(2):101-14.
⁴⁷⁰ Hauzenberger D, Olivier P, Gundersen D, Ruegg C. Tenascin-C inhibits beta I integrin-dependent T lymphocyte adhesion to fibronectin through the binding of its fnIII 1-5 repeats to fibronectin. Eur J. Immunol. 1999 May; 29(5):1435-47.
⁴⁷¹ Huang W, Chiquet-Ehrismann R, Moyano J V, Garcia-Pardo A, Orend G. Interference of tenascin-C with syndecan-4 binding to fibronectin blocks cell adhesion and stimulates tumor cell proliferation. Cancer Res. 2001 Dec. 1; 61(23):8586-94.
⁴⁷² Pesheva P, Probstmeier R, Skubitz A P, McCarthy J B, Furcht L T, Schachner M. Tenascin-R (JI 160/180) inhibits fibronectin-mediated cell adhesion—functional relatedness to tenascin-C. J Cell Sci. 1994 August; 107 (Pt 8):2323-33.
⁴⁷³ Bourdon M A, Ruoslahti E. Tenascin mediates cell attachment through an RGD-dependent receptor. J. Cell Biol. 1989 March; 108(3):1149-55.
⁴⁷⁴ Chiquet-Ehrismann R, Kalla P, Pearson C A, Beck K, Chliquet M. Tenascin interferes with fibronectin action. Cell. 1988 May 6; 53(3):383-90.
⁴⁷⁵ Doane K J, Bhattacharya R, Marchant J. Pertubation of beta1 integrin function using anti-sense or function-blocking antibodies on corneal cells grown on fibronectin and tenascin. Cell Biol Int. 2002; 26(2):131-44.
⁴⁷⁶ Deryugina E I, Bourdon M A. Tenascin mediates human glioma cell migration and modulates cell migration on fibronectin. J Cell Sci. 1996 March; 109 (Pt 3):643-52.
⁴⁷⁷ Andresen J L, Ledet T, Hager H, Josephsen K, Ehlers N. The influence of corneal stromal matrix proteins on the migration of human corneal fibroblasts. Exp Eye Res. 2000 July; 71(1):33-43.
⁴⁷⁸ Midwood K S, Schwarzbauer J E. Tenascin-C modulates matrix contraction via focal adhesion kinase- and Rho-mediated signaling pathways. Mol Biol Cell. 2002 October; 13(10):3601-13.
⁴⁷⁹ Wallner K, Li C, Shah P K, Fishbein M C, Forrester J S, Kaul S, Sharifi B G. Tenascin-C is expressed in macrophage-rich human coronary atherosclerotic plaque. Circulation. 1999 Mar. 16; 99(10):1284-9.
⁴⁸⁰ Yegin O. Chemotaxis in childhood. Pediatr Res. 1983 March; 17(3):183-7.
⁴⁸¹ Stary H C. Evolution and progression of atherosclerotic lesions in coronary arteries of children and young adults. Arteriosclerosis. 1989January-February; 9(1 Suppl):119-32.
⁴⁸² Oeth P, Mackman N. Salicylates inhibit lipopolysaccharide-induced transcriptional activation of the tissue factor gene in human monocytic cells. Blood. 1995 Dec. 1; 86(11):4144-52.
⁴⁸³ Osnes L T, Foss K B, Joo G B, Okkenhaug C, Westvik A B, Ovstebo R, Kierulf P. Acetylsalicylic acid and sodium salicylate inhibit LPS-induced NF-kappa B/c-Rel nuclear translocation, and synthesis of tissue factor (TF) and tumor necrosis factor alfa (TNF-alpha) in human monocytes. Thromb Haemost. 1996 December; 76(6):970-6.
⁴⁸⁴ Osnes L T, Haug K B, Joo G B, Westvik A B, Ovstebo R, Kierulf P. Aspirin potentiates LPS-induced fibrin formation (FPA) and TNF-alpha-synthesis in whole blood. Thromb Haemost. 2000 June; 83(6):868-73.
⁴⁸⁵ Osterud B, Olsen J O, Wilsgard L. Increased lipopolysaccharide-induced tissue factor activity and tumour necrosis factor production in monocytes after intake of aspirin: possible role of prostaglandin E2. Blood Coagul Fibrinolysis. 1992 June; 3(3):309-13.
⁴⁸⁶ Matetzky S, Tani S, Kangavari S, Dimayuga P, Yano J, Xu H, Chyu K Y, Fishbein M C, Shah P K, Cercek B. Smoking increases tissue factor expression in atherosclerotic plaques: implications for plaque thrombogenicity. Circulation. 2000 Aug. 8; 102(6):602-4.
⁴⁸⁷ Brown K A, Collins A J. Action of nonsteroidal, anti-inflammatory drugs on human and rat peripheral leucocyte migration in vitro. Ann Rheum Dis. 1977 June; 36(3):239-43.
⁴⁸⁸ Brown K A, Collins A J. In vitro effects of non-steroidal anti-inflammatory drugs on human polymorphonuclear cells and lymphocyte migration. Br J. Pharmacol. 1978 November; 64(3):347-52.
⁴⁸⁹ Egger G, Burda A, Obernosterer A, Mitterhammer H, Kager G, Jurgens G, Hofer H P, Fabjan J S, Pilger E. Blood polymorphonuclear leukocyte activation in atherosclerosis: effects of aspirin. Inflammation. 2001 April; 25(2): 129-35.
⁴⁹⁰ Higgs G A, Eakins K E, Mugridge K G, Moncada S, Vane J R. The effects of non-steroid anti-inflammatory drugs on leukocyte migration in carrageenin-induced inflammation. Eur J. Pharmacol. 1980 Aug. 22; 66(1):81-6.
⁴⁹¹ Cyrus T, Sung S, Zhao L, Funk C D, Tang S, Pratico D. Effect of low-dose aspirin on vascular inflammation, plaque stability, and atherogenesis in low-density lipoprotein receptor-deficient mice. Circulation. 2002 Sep. 3; 106(10):1282-7.
⁴⁹² Schonbeck U, Mach F, Sukhova G K, Herman M, Graber P, Kehry M R, Libby P. CD40 ligation induces tissue factor expression in human vascular smooth muscle cells. Am J Pathol. 2000 January; 156(1):7-14.
⁴⁹³ Mach F, Schonbeck U, Bonnefoy J Y, Pober J S, Libby P. Activation of monocyte/macrophage functions related to acute atheroma complication by ligation of CD40: induction of collagenase, stromelysin, and tissue factor. Circulation. 1997 Jul. 15; 96(2):396-9.
⁴⁹⁴ Lutgens E, Cleutjens K B, Heeneman S, Koteliansky V E, Burkly L C, Daemen M J. Both early and delayed anti-CD40L antibody treatment induces a stable plaque phenotype. Proc Natl Acad Sci USA. 2000 Jun. 20; 97(13):7464-9.
⁴⁹⁵ Schonbeck U, Sukhova G K, Shimizu K, Mach F, Libby P. Inhibition of CD40 signaling limits evolution of established atherosclerosis in mice. Proc Natl Acad Sci USA. 2000 Jun. 20; 97(13):7458-63.
⁴⁹⁶ Mach F, Schonbeck U, Sukhova G K, Atkinson E, Libby P. Reduction of atherosclerosis in mice by inhibition of CD40 signalling. Nature. 1998 Jul. 9; 394(6689):200-3.
⁴⁹⁷ Lutgens E, Gorelik L, Daemen M J, de Muinck E D, Grewal I S, Koteliansky V E, Flavell R A. Requirement for CD154 in the progression of atherosclerosis. Nat Med. 1999 November; 5(11):1313-6.
⁴⁹⁸ Viinikainen A, Nyman T, Fyhrquist F, Saijonmaa O. Downregulation of angiotensin converting enzyme by TNF-alpha in differentiating human macrophages. Cytokine. 2002 Jun. 21; 18(6):304-10.
⁴⁹⁹ Diet F, Pratt R E, Berry G J, Momose N, Gibbons G H, Dzau V J. Increased accumulation of tissue ACE in human atherosclerotic coronary artery disease. Circulation. 1996 Dec. 1; 94(11):2756-67.
⁵⁰⁰ Aschoff J M, Lazarus D, Fanburg B L, Lanzillo J J. Relative quantification of angiotensin-converting enzyme mRNA in human smooth muscle cells, monocytes, and lymphocytes by the polymerase chain reaction. Anal Biochem. 1994 June; 219(2):218-23.
⁵⁰¹ Lazarus D S, Aschoff J, Fanburg B L, Lanzillo J J. Angiotensin converting enzyme (kininase II) mRNA production and enzymatic activity in human peripheral blood monocytes are induced by GM-CSF but not by other cytokines. Biochim Biophys Acta. 1994 Apr. 12; 1226(1): 12-8.
⁵⁰² Unger T. The role of the renin-angiotensin system in the development of cardiovascular disease. Am J Cardiol. 2002 Jan. 24; 89(2A):3A-9A; discussion 10A. Review.
⁵⁰³ Tham D M, Martin-McNulty B, Wang Y X, Wilson D W, Vergona R, Sullivan M E, Dole W, Rutledge J C. Angiotensin II is associated with activation of NF-kappaB-mediated genes and downregulation of PPARs. Physiol Genomics. 2002 Oct. 2; 1 l(1):21-30.
⁵⁰⁴ Wolf G, Wenzel U, Burns K D, Harris R C, Stahl R A, Thaiss F. Angiotensin II activates nuclear transcription factor-kappaB through AT1 and AT2 receptors. Kidney Int. 2002 June; 61(6):1986-95.
⁵⁰⁵ Diep Q N, El Mabrouk M, Cohn J S, Endemann D, Amiri F, Virdis A, Neves M F, Schiffrin E L. Structure, endothelial function, cell growth, and inflammation in blood vessels of angiotensin 11-infused rats: role of peroxisome proliferator-activated receptor-gamma. Circulation. 2002 May 14; 105(19):2296-302.
⁵⁰⁶ Chen H, Li D, Mehta J L. Modulation of matrix metalloproteinase-1, its tissue inhibitor, and nuclear factor-kappa B by losartan in hypercholesterolemic rabbits. J Cardiovasc Pharmacol. 2002 March; 39(3):332-9.
⁵⁰⁷ Theuer J, Dechend R, Muller D N, Park J K, Fiebeler A, Barta P, Ganten D, Haller H, Dietz R, Luft F C. Angiotensin II induced inflammation in the kidney and in the heart of double transgenic rats. BMC Cardiovasc Disord. 2002; 2(1):3.
⁵⁰⁸ Muller D N, Dechend R, Mervaala E M, Park J K, Schmidt F, Fiebeler A, Theuer J, Breu V, Ganten D, Haller H, Luft F C. NF-kappaB inhibition ameliorates angiotensin II-induced inflammatory damage in rats. Hypertension. 2000 January; 35(1 Pt 2):193-201.
⁵⁰⁹ Muller D N, Mervaala E M, Schmidt F, Park J K, Dechend R, Genersch E, Breu V, Loffler B M, Ganten D, Schneider W, Haller H, Luft F C. Effect of bosentan on NF-kappaB, inflammation, and tissue factor in angiotensin II-induced end-organ damage. Hypertension. 2000 August; 36(2):282-90.
⁵¹⁰ Muller D N, Mervaala E M, Dechend R, Fiebeler A, Park J K, Schmidt F, Theuer J, Breu V, Mackman N, Luther T, Schneider W, Gulba D, Ganten D, Haller H, Luft F C. Angiotensin II (AT(1)) receptor blockade decreases vascular tissue factor in angiotensin II-induced cardiac vasculopathy. Am J Pathol. 2000 July; 157(1):111-22.
⁵¹¹ Dechend R, Fiebeler A, Park J K, Muller D N, Theuer J, Mervaala E, Bieringer M, Gulba D, Dietz R, Luft F C, Haller H. Amelioration of angiotensin II-induced cardiac injury by a 3-hydroxy-3-methylglutaryl coenzyme a reductase inhibitor. Circulation. 2001 Jul. 31; 104(5):576-81.
⁵¹² Dechend R, Fiebler A, Lindschau C, Bischoff H, Muller D, Park J K, Dietz R, Haller H, Luft F C. Modulating angiotensin 11-induced inflammation by HMG Co-A reductase inhibition. Am J Hypertens. 2001 June; 14(6 Pt 2):55S-61S. Review.
⁵¹³ Gomez-Garre D, Largo R, Tejera N, Fortes J, Manzarbeitia F, Egido J. Activation of NF-kappaB in tubular epithelial cells of rats with intense proteinuria: role of angiotensin II and endothelin-1. Hypertension. 2001 Apr.; 37(4):1171-8.
⁵¹⁴ Ruiz-Ortega M, Lorenzo O, Ruperez M, Suzuki Y, Egido J. Angiotensin II activates nuclear transcription factor-kappaB in aorta of normal rats and in vascular smooth muscle cells of AT1 knockout mice. Nephrol Dial Transplant. 2001; 16 Suppl 1:27-33.
⁵¹⁵ Ruiz-Ortega M, Lorenzo O, Ruperez M, Blanco J, Egido J. Systemic infusion of angiotensin II into normal rats activates nuclear factor-kappaB and AP-1 in the kidney: role of AT(1) and AT(2) receptors. Am J Pathol. 2001 May; 158(5):1743-56.
⁵¹⁶ Ruiz-Ortega M, Lorenzo O, Ruperez M, Konig S, Wittig B, Egido J. Angiotensin II activates nuclear transcription factor kappaB through AT(1) and AT(2) in vascular smooth muscle cells: molecular mechanisms. Circ Res. 2000 Jun. 23; 86(12):1266-72.
⁵¹⁷ Ruiz-Ortega M, Lorenzo O, Egido J: Angiotensin III increases MCP-1 and activates NF-kappaB and AP-1 in cultured mesangial and mononuclear cells. Kidney Int. 2000 June; 57(6):2285-98.
⁵¹⁸ Brasier A R, Jamaluddin M, Han Y, Patterson C, Runge M S. Angiotensin II induces gene transcription through cell-type-dependent effects on the nuclear factor-kappaB (NF-kappaB) transcription factor. Mol Cell Biochem. 2000 September; 212(1-2):155-69. Review.
⁵¹⁹ Rouet-Benzineb P, Gontero B, Dreyfus P, Lafuma C. Angiotensin II induces nuclear factor-kappa B activation in cultured neonatal rat cardiomyocytes through protein kinase C signaling pathway. J Mol Cell Cardiol. 2000 October; 32(10):1767-78.
⁵²⁰ Park J K, Muller D N, Mervaala E M, Dechend R, Fiebeler A, Schmidt F, Bieringer M, Schafer O, Lindschau C, Schneider W, Ganten D, Luft F C, Haller H. Cerivastatin prevents angiotensin II-induced renal injury independent of blood pressure-and cholesterol-lowering effects. Kidney Int. 2000 October; 58(4):1420-30.
⁵²¹ Hernandez-Presa M, Bustos C, Ortego M, Tunon J, Renedo G, Ruiz-Ortega M, Egido J. Angiotensin-converting enzyme inhibition prevents arterial nuclear factor-kappa B activation, monocyte chemoattractant protein-1 expression, and macrophage infiltration in a rabbit model of early accelerated atherosclerosis. Circulation. 1997 Mar. 18; 95(6):1532-41.
⁵²² Hernandez-Presa M A, Bustos C, Ortego M, Tunon J, Ortega L, Egido J. ACE inhibitor quinapril decreases the arterial expression of NF-kappaB-dependent proinflammatory factors but not of collagen I in a rabbit model of atherosclerosis. Am J Pathol. 1998 December; 153(6):1825-37.
⁵²³ Napoleone E, Di Santo A, Camera M, Tremoli E, Lorenzet R. Angiotensin-converting enzyme inhibitors downregulate tissue factor synthesis in monocytes. Circ Res. 2000 Feb. 4; 86(2):139-43.
⁵²⁴ Nagata K, Ishibashi T, Sakamoto T, Nakazato K, Seino Y. Yokoyama K, Ohkawara H, Teramoto T, Maruyama Y. Effects of blockade of the renin-angiotensin system on tissue factor and plasminogen activator inhibitor-1 synthesis in human cultured monocytes. J Hypertens. 2001 April; 19(4):775-83.
⁵²⁵ Zaman A K, Fujii S, Sawa H, Goto D, Ishimori N, Watano K, Kaneko T, Furumoto T, Sugawara T, Sakuma 1, Kitabatake A, Sobel B E. Angiotensin-converting enzyme inhibition attenuates hypofibrinolysis and decreases cardiac perivascular fibrosis in genetically obese diabetic mice. Circulation. 2001 Jun. 26; 103(25):3123-8.
⁵²⁶ Soejima H, Ogawa H, Yasue H, Suefuji H, Kaikita K, Tsuji I, Kumeda K, Aoyama N. Effects of enalapril on tissue factor in patients with uncomplicated acute myocardial infarction. Am J Cardiol. 1996 Aug. 1; 78(3):336-40.
⁵²⁷ Soejima H, Ogawa H, Yasue H, Kaikita K, Takazoe K, Nishiyama K, Misumi K, Miyamoto S, Yoshimura M, Kugiyama K, Nakamura S, Tsuji I. Angiotensin-converting enzyme inhibition decreases monocyte chemoattractant protein-1 and tissue factor levels in patients with myocardial infarction. J Am Coll Cardiol. 1999 October; 34(4):983-8.
⁵²⁸ Soejima H, Ogawa H, Suefuji H, Kaikita K, Takazoe K, Miyamoto S, Kajiwara I, Shimomura H, Sakamoto T, Yoshimura M, Nakamura S. Comparison of effects of losartan versus enalapril on fibrinolysis and coagulation in patients with acute myocardial infarction. Am J Cardiol. 2001 Jun. 15; 87(12):1408-11.
⁵²⁹ Elferink J G, de Koster B M. The stimulation of human neutrophil migration by angiotensin IL: its dependence on Ca2+ and the involvement of cyclic GMP. Br J. Pharmacol. 1997 June; 121(4):643-8.
⁵³⁰ Liu G, Espinosa E, Oemar B S, Luscher T F. Bimodal effects of angiotensin II on migration of human and rat smooth muscle cells. Direct stimulation and indirect inhibition via transforming growth factor-beta 1. Arterioscler Thromb Vasc Biol. 1997 July; 17(7):1251-7.
⁵³¹ Hoshida S, Nishida M, Yamashita N, Igarashi J, Aoki K, Hori M, Kuzuya T, Tada M. Vascular angiotensin-converting enzyme activity in cholesterol-fed rabbits: effects of enalapril. Atherosclerosis. 1997 April; 130(1-2):53-9.
⁵³² Kowala M C, Valentine M, Recce R, Beyer S, Goller N, Durham S, Aberg G. Enhanced reduction of atherosclerosis in hamsters treated with pravastatin and captopril: ACE in atheromas provides cellular targets for captopril. J Cardiovasc Pharmacol. 1998 July; 32(1):29-38.
⁵³³ Ohishi M, Ueda M, Rakugi H, Okamura A, Naruko T, Becker A E, Hiwada K, Kamitani A, Kamide K, Higaki J, Ogihara T. Upregulation of angiotensin-converting enzyme during the healing process after injury at the site of percutaneous transluminal coronary angioplasty in humans. Circulation. 1997 Nov. 18; 96(10):3328-37.
⁵³⁴ Daugherty A, Manning M W, Cassis L A. Angiotensin II promotes atherosclerotic lesions and aneurysms in apolipoprotein E-deficient mice. J Clin Invest. 2000 June; 105(11):1605-12.
⁵³⁵ Allaire E, Muscatelli-Groux B, Mandet C, Guinault A M, Bruneval P, Desgranges P, Clowes A, Melliere D, Becquemin J P. Paracrine effect of vascular smooth muscle cells in the prevention of aortic aneurysm formation. J Vasc Surg. 2002 November; 36(5):1018-26.
⁵³⁶ Theocharis A D, Tsolakis I, Hjerpe A, Karamanos N K. Human abdominal aortic aneurysm is characterized by decreased versican concentration and specific downregulation of versican isoform V(O). Atherosclerosis. 2001 Feb. 1; 154(2):367-76.
⁵³⁷ Raymond J, Desfaits A C, Roy D. Fibrinogen and vascular smooth muscle cell grafts promote healing of experimental aneurysms treated by embolization. Stroke. 1999 August; 30(8):1657-64.
⁵³⁸ Raymond J, Venne D, Alias S, Roy D, Oliva V L, Denbow N, Salazkin I, Leclerc G. Healing mechanisms in experimental aneurysms. I. Vascular smooth muscle cells and neointima formation. J Neuroradiol. 1999 March; 26(1):7-20.
⁵³⁹ Keidar S, Attias J, Heinrich R, Coleman R, Aviram M. Angiotensin II atherogenicity in apolipoprotein E deficient mice is associated with increased cellular cholesterol biosynthesis. Atherosclerosis. 1999 October; 146(2):249-57.
⁵⁴⁰ Warnholtz A, Nickenig G, Schulz E, Macharzina R, Brasen J H, Skatchkov M, Heitzer T, Stasch J P, Griendling K K, Harrison D G, Bohm M, Meinertz T, Munzel T. Increased NADH-oxidase-mediated superoxide production in the early stages of atherosclerosis: evidence for involvement of the renin-angiotensin system. Circulation. 1999 Apr. 20; 99(15):2027-33.
⁵⁴¹ de Nigris F, D'Armiento F P, Somma P, Casini A, Andreini I, Sarlo F, Mansueto G, De Rosa G, Bonaduce D, Condorelli M, Napoli C. Chronic treatment with sulfhydryl angiotensin-converting enzyme inhibitors decrease susceptibility of plasma LDL to in vitro oxidation, formation of oxidation-specific epitopes in the arterial wall, and atherogenesis in apolipoprotein E knockout mice. Int J Cardiol. 2001 December; 81(2-3):107-15; discusssion 115-6.
⁵⁴² Keidar S, Attias J, Coleman R, Wirth K, Scholkens B, Hayek T. Attenuation of atherosclerosis in apolipoprotein E-deficient mice by ramipril is dissociated from its antihypertensive effect and from potentiation of bradykinin. J Cardiovasc Pharmacol. 2000 January; 35(1):64-72.
⁵⁴³ Kowala M C, Recce R, Beyer S, Aberg G. Regression of early atherosclerosis in hyperlipidemic hamsters induced by fosinopril and captopril. J Cardiovasc Pharmacol. 1995 February; 25(2):179-86.
⁵⁴⁴ Napoli C, Cicala C, D'Armiento F P, Roviezzo F, Somma P, de Nigris F, Zuliani P, Bucci M, Aleotti L, Casini A, Franconi F, Cirino G. Beneficial effects of ACE-inhibition with zofenopril on plaque formation and low-density lipoprotein oxidation in watanabe heritable hyperlipidemic rabbits. Gen Pharmacol. 1999 December; 33(6):467-77.
⁵⁴⁵ Yusuf S, Sleight P, Pogue J, Bosch J, Davies R, Dagenais G. Effects of an angiotensin-converting-enzyme inhibitor, ramipril, on cardiovascular events in high-risk patients. The Heart Outcomes Prevention Evaluation Study Investigators. N Engl J. Med. 2000 Jan. 20; 342(3):145-53.
⁵⁴⁶ MacMahon S, Sharpe N, Gamble G, Clague A, Mhurchu C N, Clark T, Hart H, Scott J, White H. Randomized, placebo-controlled trial of the angiotensin-converting enzyme inhibitor, ramipril, in patients with coronary or other occlusive arterial disease. PART-2 Collaborative Research Group. Prevention of Atherosclerosis with Ramipril. J Am Coll Cardiol. 2000 August; 36(2):438-43.
⁵⁴⁷ Teo K K, Burton J R, Buller C E, Plante S, Catellier D, Tymchak W, Dzavik V, Taylor D, Yokoyama S, Montague T J. Long-term effects of cholesterol lowering and angiotensin-converting enzyme inhibition on coronary atherosclerosis: The Simvastatin/Enalapril Coronary Atherosclerosis Trial (SCAT). Circulation. 2000 Oct. 10; 102(15):1748-54.
⁵⁴⁸ Lonn E, Yusuf S, Dzavik V, Doris C, Yi Q, Smith S, Moore-Cox A, Bosch J, Riley W, Teo K; SECURE Investigators. Effects of ramipril and vitamin E on atherosclerosis: the study to evaluate carotid ultrasound changes in patients treated with ramipril and vitamin E (SECURE). Circulation. 2001 Feb. 20; 103(7):919-25.
⁵⁴⁹ Halkin A, Keren G. Potential indications for angiotensin-converting enzyme inhibitors in atherosclerotic vascular disease. Am J. Med. 2002 Feb. 1; 112(2): 126-34. Review.
⁵⁵⁰ Takemoto M, Liao J K. Pleiotropic effects of 3-hydroxy-3-methylglutaryl coenzyme a reductase inhibitors. Arterioscler Thromb Vasc Biol. 2001 November; 21(11):1712-9. Review.
⁵⁵¹ Reyes-Reyes M, Mora N, Zentella A, Rosales C. Phosphatidylinositol 3-kinase mediates integrin-dependent NF-kappaB and MAPK activation through separate signaling pathways. J Cell Sci. 2001 April; 114(Pt 8):1579-89.
⁵⁵² Guha M, O'Connell M A, Pawlinski R, Hollis A, McGovern P, Yan S F, Stern D, Mackman N. Lipopolysaccharide activation of the MEK-ERK1/2 pathway in human monocytic cells mediates tissue factor and tumor necrosis factor alpha expression by inducing Elk-1 phosphorylation and Egr-1 expression. Blood. 2001 Sep. 1; 98(5):1429-39.
⁵⁵³ Golovchenko I, Goalstone M L, Watson P, Brownlee M, Draznin B. Hyperinsulinemia enhances transcriptional activity of nuclear factor-kappaB induced by angiotensin II, hyperglycemia, and advanced glycosylation end products in vascular smooth muscle cells. Circ Res. 2000 Oct. 27; 87(9):746-52.
⁵⁵⁴ Montaner S, Perona R, Saniger L, Lacal J C. Activation of serum response factor by RhoA is mediated by the nuclear factor-kappaB and C/EBP transcription factors. J Biol. Chem. 1999 Mar. 26; 274(13):8506-15.
⁵⁵⁵ Montaner S, Perona R, Saniger L, Lacal J C. Multiple signalling pathways lead to the activation of the nuclear factor kappaB by the Rho family of GTPases. J Biol. Chem. 1998 May 22; 273(21):12779-85.
⁵⁵⁶ Hernandez-Presa M A, Ortego M, Tunon J, Marti;n-Ventura J L, Mas S, Blanco-Colio L M, Aparicio C, Ortega L, Gomez-Gerique J, Vivanco F, Egido J. Simvastatin decreases NF-kappaB activity in peripheral mononuclear and in plaque cells of rabbit atheroma more markedly than lipid lowering diet. Cardiovasc Res. 2003 January; 57(1):168-177.
⁵⁵⁷ Inoue I, Itoh F, Aoyagi S, Tazawa S, Kusama H, Akahane M, Mastunaga T, Hayashi K, Awata T, Komoda T, Katayama S. Fibrate and statin synergistically increase the transcriptional activities of PPARalpha/RXRalpha and decrease the transactivation of NFkappaB. Biochem Biophys Res Commun. 2002 Jan. 11; 290(1): 131-9.
⁵⁵⁸ Zelvyte I, Dominaitiene R, Crisby M, Janciauskiene S. Modulation of inflammatory mediators and PPARgamma and NFkappaB expression by pravastatin in response to lipoproteins in human monocytes in vitro. Pharmacol Res. 2002 February; 45(2): 147-54.
⁵⁵⁹ Rasmussen L M, Hansen P R, Nabipour M T, Olesen P, Kristiansen M T, Ledet T. Diverse effects of inhibition of 3-hydroxy-3-methylglutaryl-CoA reductase on the expression of VCAM-1 and E-selectin in endothelial cells. Biochem J. 2001 Dec. 1; 360(Pt 2):363-70.
⁵⁶⁰ Teupser D, Bruegel M, Stein O, Stein Y, Thiery J. HMG-CoA reductase inhibitors decrease adhesion of human monocytes to endothelial cells. Biochem Biophys Res Commun. 2001 Dec. 14; 289(4):838-44.
⁵⁶¹ Ortego M, Bustos C, Hernandez-Presa M A, Tunon J, Diaz C, Hernandez G, Egido J. Atorvastatin decreases NF-kappaB activation and chemokine expression in vascular smooth muscle cells and mononuclear cells. Atherosclerosis. 1999 December; 147(2):253-61.
⁵⁶² Bustos C, Hernandez-Presa M A, Ortego M, Tunon J, Ortega L, Perez F, Diaz C, Hernandez G, Egido J. HMG-CoA reductase inhibition by atorvastatin decreases neointimal inflammation in a rabbit model of atherosclerosis. J Am Coll Cardiol. 1998 December; 32(7):2057-64.
⁵⁶³ Nagata K, Ishibashi T, Sakamoto T, Ohkawara H, Shindo J, Yokoyama K, Sugimoto K, Sakurada S, Takuwa Y, Nakamura S, Teramoto T. Maruyama Y. Rho/Rho-kinase is involved in the synthesis of tissue factor in human monocytes. Atherosclerosis. 2002 July; 163(1):39-47.
⁵⁶⁴ Ferro D, Basili S, Alessandri C, Cara D, Violi F. Inhibition of tissue-factor-mediated thrombin generation by simvastatin. Atherosclerosis. 2000 March; 149(1):111-6.
⁵⁶⁵ Colli S, Eligini S. Lalli M, Camera M, Paoletti R, Tremoli E. Vastatins inhibit tissue factor in cultured human macrophages. A novel mechanism of protection against atherothrombosis. Arterioscler Thromb Vasc Biol. 1997 February; 17(2):265-72.
⁵⁶⁶ Baetta R, Camera M, Comparato C, Altana C, Ezekowitz M D, Tremoli E. Fluvastatin decreases tissue factor expression and macrophage accumulation in carotid lesions of cholesterol-fed rabbits in the absence of lipid lowering. Arterioscler Thromb Vasc Biol. 2002 Apr. 11; 22(4):692-8.
⁵⁶⁷ Sukhova G K, Williams J K, Libby P. Statins decrease inflammation in atheroma of nonhuman primates independent of effects on serum cholesterol. Arterioscler Thromb Vasc Biol. 2002 Sep. 1; 22(9):1452-8.
⁵⁶⁸ Aikawa M, Rabkin E, Sugiyama S, Voglic S J, Fukumoto Y, Furukawa Y, Shiomi M, Schoen F J, Libby P. An HMG-CoA Reductase Inhibitor, Cerivastatin, Suppresses Growth of Macrophages Expressing Matrix Metalloproteinases and Tissue Factor In Vivo and In Vitro. Circulation. 2001 Jan. 16; 103(2):276-283.
⁵⁶⁹ Libby P, Aikawa M. Stabilization of atherosclerotic plaques: new mechanisms and clinical targets. Nat Med. 2002 November; 8(11)1257-62. Review.
⁵⁷⁰ Holschermann H, Terhalle H M, Zakel U, Maus U, Parviz B, Tillmanns H, Haberbosch W. Monocyte tissue factor expression is enhanced in women who smoke and use oral contraceptives. Thromb Haemost. 1999 December; 82(6): 1614-20.
⁵⁷¹ Simons L A, Simons J, Friedlander Y, McCallum J, Palaniappan L. Risk functions for prediction of cardiovascular disease in elderly Australians: the Dubbo Study. Med J Aust. 2003 Feb. 3; 178(3): 113-6.
⁵⁷² Jee S H, Suh I, Kim I S, Appel L J. Smoking and atherosclerotic cardiovascular disease in men with low levels of serum cholesterol: the Korea Medical Insurance Corporation Study. JAMA. 1999 Dec. 8; 282(22):2149-55.
⁵⁷³ Kawachi I, Colditz G A. Workplace exposure to passive smoking and risk of cardiovascular disease: summary of epidemiologic studies. Environ Health Perspect. 1999 December; 107 Suppl 6:847-51. Review.
⁵⁷⁴ Iribarren C, Tekawa I S, Sidney S, Friedman G D. Effect of cigar smoking on the risk of cardiovascular disease, chronic obstructive pulmonary disease, and cancer in men. N Engl J. Med. 1999 Jun. 10; 340(23):1773-80.
⁵⁷⁵ He J, Vupputuri S, Allen K, Prerost M R, Hughes J, Whelton P K. Passive smoking and the risk of coronary heart disease—a meta-analysis of epidemiologic studies. N Engl J. Med. 1999 Mar. 25; 340(12):920-6.
⁵⁷⁶ Ockene I S, Miller N H. Cigarette smoking, cardiovascular disease, and stroke: a statement for healthcare professionals from the American Heart Association. American Heart Association Task Force on Risk Reduction. Circulation. 1997 Nov. 4; 96(9):3243-7.
⁵⁷⁷ Blanco-Colio L M, Valderrama M, Alvarez-Sala L A, Bustos C, Ortego M, Hernandez-Presa M A, Cancelas P, Gomez-Gerique J, Millan J, Egido J. Red wine intake prevents nuclear factor-kappaB activation in peripheral blood mononuclear cells of healthy volunteers during postprandial lipemia. Circulation. 2000 Aug. 29; 102(9):1020-6.
⁵⁷⁸ de Gaetano G, Cerletti C; European project. FAIR CT 97 3261 Project participants. Wine and cardiovascular disease. Nutr Metab Cardiovasc Dis. 2001 August; 11(4 Suppl):47-50. Review.
⁵⁷⁹ Rotondo S, Di Castelnuovo A, de Gaetano G. The relation between wine consumption and cardiovascular risk: from epidemiological evidence to biological plausibility. Ital Heart J. 2001 January; 2(1):1-8. Review.
⁵⁸⁰ Sato M, Maulik N, Das D K. Cardioprotection with alcohol: role of both alcohol and polyphenolic antioxidants. Ann N Y Acad. Sci. 2002 May; 957:122-35.
⁵⁸¹ Wollin S D, Jones P J. Alcohol, red wine and cardiovascular disease. J Nutr. 2001 May; 131(5):1401-4. Review.
⁵⁸² Langer C, Huang Y, Cullen P, Wiesenhutter B, Mahley R W, Assmann G, von Eckardstein A. Endogenous apolipoprotein E modulates cholesterol efflux and cholesteryl ester hydrolysis mediated by high-density lipoprotein-3 and lipid-free apolipoproteins in mouse peritoneal macrophages. J Mol Med. 2000; 78(4):217-27.
⁵⁸³ Mazzone T, Reardon C. Expression of heterologous human apolipoprotein E by J774 macrophages enhances cholesterol efflux to HDL3. J Lipid Res. 1994 August; 35(8):1345-53.
⁵⁸⁴ Huang Y, von Eckardstein A, Wu S, Maeda N, Assmann G. A plasma lipoprotein containing only apolipoprotein E and with gamma mobility on electrophoresis releases cholesterol from cells. Proc Natl Acad Sci USA. 1994 Mar. 1; 91(5):1834-8.
⁵⁸⁵ Tsukamoto K, Tangirala R, Chun S H, Pure E, Rader D J. Rapid regression of atherosclerosis induced by liver-directed gene transfer of ApoE in ApoE-deficient mice. Arterioscler Thromb Vasc Biol. 1999 September; 19(9):2162-70.
⁵⁸⁶ Wilson S H, Best P J, Edwards W D, Holmes D R Jr, Carlson P J, Celermajer D S, Lerman A. Nuclear factor-kappaB immunoreactivity is present in human coronary plaque and enhanced in patients with unstable angina pectoris. Atherosclerosis. 2002 January; 160(1):147-53.
⁵⁸⁷ Westmuckett A D, Lupu C, Goulding D A, Das S, Kakkar V V, Lupu F. In situ analysis of tissue factor-dependent thrombin generation in human atherosclerotic vessels. Thromb Haemost. 2000 November; 84(5):904-11.
⁵⁸⁸ Crawley J, Lupu F, Westmuckett A D, Severs N J, Kakkar V V, Lupu C. Expression, localization, and activity of tissue factor pathway inhibitor in normal and atherosclerotic human vessels. Arterioscler Thromb Vasc Biol. 2000 May; 20(5):1362-73.
⁵⁸⁹ Kaikita K, Takeya M, Ogawa H, Suefuji H, Yasue H, Takahashi K. Co-localization of tissue factor and tissue factor pathway inhibitor in coronary atherosclerosis. J Pathol. 1999 June; 188(2): 180-8.
⁵⁹⁰ Hatakeyama K, Asada Y, Marutsuka K, Sato Y, Kamikubo Y, Sumiyoshi A. Localization and activity of tissue factor in human aortic atherosclerotic lesions. Atherosclerosis. 1997 September; 133(2):213-9.
⁵⁹¹ Kato K, Elsayed Y A, Namoto M, Nakagawa K, Sueishi K. Enhanced expression of tissue factor activity in the atherosclerotic aortas of cholesterol-fed rabbits. Thromb Res. 1996 May 15; 82(4):335-47.
⁵⁹² Sueishi K, Ichikawa K, Nakagawa K, Kato K, Elsayed Y A, Namoto M. Procoagulant properties of atherosclerotic aortas. Ann N Y Acad. Sci. 1995 Jan. 17; 748:185-92; discussion 192-3.
⁵⁹³ Landers S C, Gupta M, Lewis J C. Ultrastructural localization of tissue factor on monocyte-derived macrophages and macrophage foam cells associated with atherosclerotic lesions. Virchows Arch. 1994; 425(1):49-54.
⁵⁹⁴ Wilcox J N, Smith K M, Schwartz S M, Gordon D. Localization of tissue factor in the normal vessel wall and in the atherosclerotic plaque. Proc Natl Acad Sci USA. 1989 April; 86(8):2839-43.
⁵⁹⁵ Moons A H, Levi M, Peters R J. Tissue factor and coronary artery disease. Cardiovasc Res. 2002 Feb. 1; 53(2):313-25. Review.
⁵⁹⁶ Tremoli E, Camera M, Toschi V, Colli S. Tissue factor in atherosclerosis. Atherosclerosis. 1999 June; 144(2):273-83. Review.
⁵⁹⁷ Taubman M B, Fallon J T, Schecter A D, Giesen P, Mendlowitz M, Fyfe B S, Marmur J D, Nemerson Y. Tissue factor in the pathogenesis of atherosclerosis. Thromb Haemost. 1997 July; 78(1):200-4. Review.
⁵⁹⁸ Osterud B. Tissue factor expression by monocytes: regulation and pathophysiological roles. Blood Coagul Fibrinolysis. 1998 March; 9 Suppl 1:S9-14. Review.
⁵⁹⁹ Osterud B. Tissue factor: a complex biological role. Thromb Haemost. 1997 July; 78(1):755-8. Review.
⁶⁰⁰ Hatakeyama K, Asada Y, Marutsuka K, Kataoka H, Sato Y, Sumiyoshi A. Expression of tissue factor in the rabbit aorta after balloon injury. Atherosclerosis. 1998 August; 139(2):265-71.
⁶⁰¹ Aikawa M, Voglic S J, Sugiyama S, Rabkin E, Taubman M B, Fallon J T, Libby P. Dietary lipid lowering decreases tissue factor expression in rabbit atheroma. Circulation. 1999 Sep. 14; 100(11):1215-22.
⁶⁰² Taylor-Wiedeman J, Sissons P, Sinclair J. Induction of endogenous human cytomegalovirus gene expression after differentiation of onocytes from healthy carriers. J. Virol. 1994 March; 68(3):1597-604.
⁶⁰³ Guetta E, Guetta V, Shibutani T, Epstein S E. Monocytes harboring cytomegalovirus: interactions with endothelial cells, smooth muscle cells, and oxidized low-density lipoprotein. Possible mechanisms for activating virus delivered by monocytes to sites of vascular injury. Circ Res 1997 July; 81(1):8-16.
⁶⁰⁴ Ikuta K, Luftig R B. Inhibition of cleavage of Moloney murine leukemia virus gag and env coded precursor polyproteins by cerulenin. Virology. 1986 Oct. 15; 154(1):195-206.
⁶⁰⁵ Katoh I, Yoshinaka Y, Luftig R B. The effect of cerulenin on Moloney murine leukemia virus morphogenesis. Virus Res. 1986 August; 5(2-3):265-76.
⁶⁰⁶ Goldfine H, Harley J B, Wyke J A. Effects of inhibitors of lipid synthesis on the replication of Rous sarcoma virus. A specific effect of cerulenin on the processing of major non-glycosylated viral structural proteins. Biochim Biophys Acta 1978 Sep. 22; 512(2):22940.
⁶⁰⁷ Ibanez C E, Schrier R, Ghazal P, Wiley C, Nelson J A. Human cytomegalovirus productively infects primary differentiated macrophages. J. Virol. 1991 December; 65(12):6581-8.
⁶⁰⁸ Lathey J L, Spector S A. Unrestricted replication of human cytomegalovirus in hydrocortisone-treated macrophages. J. Virol. 1991 November; 65(11):6371-5.
⁶⁰⁹ Weinshenker B G, Wilton S, Rice G P. Phorbol ester-induced differentiation permits productive human cytomegalovirus infection in a monocytic cell line. J. Immunol. 1988 Mar. 1; 140(5):1625-31.
⁶¹⁰ Gonczol E, Andrews P W, Plotkin S A. Cytomegalovirus replicates in differentiated but not in undifferentiated human embryonal carcinoma cells. Science. 1984 Apr. 13; 224(4645):159-61.
⁶¹¹ Zhou Y F, Yu Z X, Wanishsawad C, Shou M, Epstein S E. The immediate early gene products of human cytomegalovirus increase vascular smooth muscle cell migration, proliferation, and expression of PDGF beta-receptor. Biochem Biophys Res Commun. 1999 Mar. 24; 256(3):608-13.
⁶¹² Zhou Y F, Guetta E, Yu Z X, Finkel T, Epstein S E. Human cytomegalovirus increases modified low density lipoprotein uptake and scavenger receptor mRNA expression in vascular smooth muscle cells. J Clin Invest 1996 Nov. 1; 98(9):2129-38.
⁶¹³ Tumilowicz J J, Gawlik M E, Powell B B, Trentin J J. Replication of cytomegalovirus in human arterial smooth muscle cells. J. Virol. 1985 December; 56(3):839-45.
⁶¹⁴ Melnick J L, Petrie B L, Dreesman G R, Burek J, McCollum C H, DeBakey M E. Cytomegalovirus antigen within human arterial smooth muscle cells. Lancet. 1983 Sep. 17; 2(8351):644-7.
⁶¹⁵ Benditt E P, Barrett T, McDougall J K. Viruses in the etiology of atherosclerosis. Proc Natl Acad Sci USA 1983 October; 80(20):6386-9.
⁶¹⁶ Shirasaki F, Makhluf H A, LeRoy C, Watson D K, Trojanowska M. Ets transcription factors cooperate with Sp1 to activate the human tenascin-C promoter. Oncogene. 1999 Dec. 16; 18(54):7755-64.
⁶¹⁷ Stary H C, Chandler A B, Dinsmore R E, Fuster V, Glagov S, Insull W Jr, Rosenfeld M E, Schwartz C J, Wagner W D, Wissler R W. A definition of advanced types of atherosclerotic lesions and a histological classification of atherosclerosis. A report from the Committee on Vascular Lesions of the Council on Arteriosclerosis, American Heart Association. Arterioscler Thromb Vasc Biol. 1995 September; 15(9): 1512-31.
⁶¹⁸ Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol. 2000 May; 20(5):1262-75.
⁶¹⁹ Guyton J R. The role of lipoproteins in atherogenesis. Adv Exp Med Biol. 1995; 369:29-38.
⁶²⁰ Loukas M, Dabrowski M, Wagner T, Walczak E, Witkowski A, Ruzyllo W. Fibrinogen and smooth muscle cell detection in atherosclerotic plaques from stable and unstable angina—an immunohistochemical study. Med Sci Monit. 2002 April; 8(4):BR144-8.
⁶²¹ Bauriedel G, Hutter R, Welsch U, Bach R, Sievert H, Luderitz B. Role of smooth muscle cell death in advanced coronary primary lesions: implications for plaque instability. Cardiovasc Res. 1999 February; 41(2):480-8.
⁶²² Newby A C, Zaltsman A B. Fibrous cap formation or destruction—the critical importance of vascular smooth muscle cell proliferation, migration and matrix formation. Cardiovasc Res. 1999 February; 41(2):345-60. Review.
⁶²³ Nakashima Y, Chen Y X, Kinukawa N, Sueishi K. Distributions of diffuse intimal thickening in human arteries: preferential expression in atherosclerosis-prone arteries from an early age. Virchows Arch. 2002 September; 441(3):279-88.
⁶²⁴ Stary H C, Blankenhom D H, Chandler A B, Glagov S, Insull W Jr, Richardson M, Rosenfeld M E, Schaffer S A, Schwartz C J, Wagner W D, et al. A definition of the intima of human arteries and of its atherosclerosis-prone regions. A report from the Committee on Vascular Lesions of the Council on Arteriosclerosis, American Heart Association. Circulation. 1992 January; 85(1):391-405. Review.
⁶²⁵ Pan J, Xia L, McEver R P. Comparison of promoters for the murine and human P-selectin genes suggests species-specific and conserved mechanisms for transcriptional regulation in endothelial cells. J Biol. Chem. 1998 Apr. 17; 273(16):10058-67.
⁶²⁶ Chiu B. Multiple infections in carotid atherosclerotic plaques. Am Heart J. 1999 November; 138(5 Pt 2):534-536.
⁶²⁷ Nieto F J. Viruses and atherosclerosis: A critical review of the epidemiologic evidence. Am Heart J 1999 November; 138(5 Pt 2):453-460.
⁶²⁸ Adam E, Melnick J L, Probtsfield J L, Petrie B L, Burek J, Bailey K R, McCollum C H, DeBakey M E. High levels of cytomegalovirus antibody in patients requiring vascular surgery for atherosclerosis. Lancet. 1987 Aug. 8; 2(8554):291-3.
⁶²⁹ Li B, Xu C, Wang Q. The detection of the antibodies of human cytomegalovirus in the sera of patients with coronary heart disease. Chung Hua Nei Ko Tsa Chih. 1996 November; 35(11):741-3. (in Chinese).
⁶³⁰ Liuzzo G, Caligiuri G, Grillo R L, et al. Helicobacter pylori and cytomegalovirus infectoins are strongly associated with atherosclerosis, but are not responsible for the instability of angina [abstract]. J Am Coll Cardiol 1997; 29(suppl A):217A.
⁶³¹ Blum A, Giladi M, Weinberg M, Kaplan G, Pasternack H, Laniado S, Miller H. High anti-cytomegalovirus (CMV) IgG antibody titer is associated with coronary artery disease and may predict post-coronary balloon angioplasty restenosis. Am J. Cardiol. 1998 Apr 1; 81(7):866-8.
⁶³² Sorlie P D, Nieto F J, Adam E, Folsom A R, Shahar E, Massing M. A prospective study of cytomegalovirus, herpes simplex virus 1, and coronary heart disease: the atherosclerosis risk in communities (ARIC) study. Arch Intern Med. 2000 Jul. 10; 160(13):2027-32.
⁶³³ Fabricant C G, Fabricant J. Atherosclerosis induced by infection with Marek's disease herpesvirus in chickens. Am Heart J 1999 November; 138(5 Pt 2):S465-8.
⁶³⁴ Dodet B, Plotkin S A. Infection and atherosclerosis. Am Heart J 1999 November; 138(5 Pt 2):417-418.
⁶³⁵ Fong I W. Emerging relations between infectious diseases and coronary artery disease and atherosclerosis. CMAJ. 2000 Jul. 11; 163(1):49-56.
⁶³⁶ zur Hausen H. Viruses in human cancers. Eur J Cancer. 1999 December; 35(14):1878-85.
⁶³⁷ Crawford L. Criteria for establishing that a virus is oncogenic. Ciba Found Symp. 1986; 120: 104-16.
⁶³⁸ Butel J S. Viral carcinogenesis: revelation of molecular mechanisms and etiology of human disease. Carcinogenesis. 2000 March; 21(3):405-26.
⁶³⁹ Donovan-Peluso M, George L D, Hassett A C. Lipopolysaccharide induction of tissue factor expression in THP-1 monocytic cells. Protein-DNA interactions with the promoter. J Biol. Chem. 1994 Jan. 14; 269(2):1361-9.
⁶⁴⁰ Groupp E R, Donovan-Peluso M. Lipopolysaccharide induction of THP-1 cells activates binding of c-Jun, Ets, and Egr-1 to the tissue factor promoter. J Biol. Chem. 1996 May 24; 271(21):12423-30.
⁶⁴¹ Holzmuller H, Moll T, Hofer-Warbinek R, Mechtcheriakova D, Binder B R, Hofer E. A transcriptional suppressor of the tissue factor gene in endothelial cells. Arterioscler Thromb Vasc Biol. 1999 July; 19(7):1804-11.
⁶⁴² Hall A J, Vos H L, Bertina R M. Lipopolysaccharide induction of tissue factor in THP-1 cells involves Jun protein phosphorylation and nuclear factor kappaB nuclear translocation. J Biol. Chem. 1999 Jan. 1; 274(1):376-83.
⁶⁴³ Moll T, Czyz M, Holzmuller H, Hofer-Warbinek R, Wagner E, Winkler H, Bach F H, Hofer E. Regulation of the tissue factor promoter in endothelial cells. Binding of NF kappa B-, AP-1-, and Sp1-like transcription factors. J Biol. Chem. 1995 Feb. 24; 270(8):3849-57:
⁶⁴⁴ Nathwani A C, Gale K M, Pemberton K D, Crossman D C, Tuddenham E G, McVey J H. Efficient gene transfer into human umbilical vein endothelial cells allows functional analysis of the human tissue factor gene promoter. Br J Haematol. 1994 September; 88(1):122-8.
⁶⁴⁵ Mackman N, Fowler B J, Edgington T S, Morrissey J H. Functional analysis of the human tissue factor promoter and induction by serum. Proc Natl Acad Sci USA. 1990 March; 87(6):2254-8.
⁶⁴⁶ Nemerson Y, Giesen P L. Some thoughts about localization and expression of tissue factor. Blood Coagul Fibrinolysis. 1998 March; 9 Suppl 1:S45-7.
⁶⁴⁷ Bach R R, Moldow C F. Mechanism of tissue factor activation on HL-60 cells. Blood. 1997 May 1; 89(9):3270-6.
⁶⁴⁸ Lammie G A, Sandercock P A, Dennis M S. Recently occluded intracranial and extracranial carotid arteries. Relevance of the unstable atherosclerotic plaque. Stroke. 1999 July; 30(7):1319-25.
⁶⁴⁹ Chambless L E, Folsom A R, Clegg L X, Sharrett A R, Shahar E, Nieto F J, Rosamond W D, Evans G. Carotid wall thickness is predictive of incident clinical stroke: the Atherosclerosis Risk in Communities (ARIC) study. Am J Epidemiol 2000 Mar. 1; 151(5):478-87.
⁶⁵⁰ O'Leary D H, Polak J F, Kronmal R A, Manolio T A, Burke G L, Wolfson S K. Carotid-artery intima and media thickness as a risk factor for myocardial infarction and stroke in older adults. Cardiovascular Health Study Collaborative Research Group. N Engl J. Med. 1999 Jan. 7; 340(1):14-22.
⁶⁵¹ Hart D N. Dendritic cells: unique leukocyte populations which control the primary immune response. Blood. 1997 Nov. 1; 90(9):3245-87.
⁶⁵² Zhuravskaya T, Maciejewski J P, Netski D M, Bruening E, Mackintosh F R, St Jeor S. Spread of human cytomegalovirus (HCMV) after infection of human hematopoietic progenitor cells: model of HCMV latency. Blood 1997 Sep. 15; 90(6):2482-91.
⁶⁵³ Maciejewski J P, St Jeor S C. Human cytomegalovirus infection of human hematopoietic progenitor cells. Leuk Lymphoma 1999 March; 33(1-2):1-13.
⁶⁵⁴ Sindre H, Tjoonnfjord G E, Rollag H, Ranneberg-Nilsen T, Veiby O P, Beck S, Degre M, Hestdal K. Human cytomegalovirus suppression of and latency in early hematopoietic progenitor cells. Blood 1996 Dec. 15; 88(12):4526-33.
⁶⁵⁵ Kappelmayer J, Berecki D, Misz M, Olah L, Fekete I, Csiba L, Blasko G. Monocytes express tissue factor in young patients with cerebral ischemia. Cerebrovasc Dis. 1998 July-August; 8(4):235-9.
⁶⁵⁶ Constant S L, Bottomly K. Induction of Th1 and Th2 CD4+ T-cell responses: the alternative approaches. Annu Rev Immunol. 1997; 15:297-322.
⁶⁵⁷ Rogers P R, Croft M. Peptide dose, affinity, and time of differentiation can contribute to the Th1/Th2 cytokine balance. J. Immunol. 1999 Aug. 1; 163(3):1205-13.
⁶⁵⁸ Rovere P, Fazzini F, Sabbadini M G, Manfredi A A. Apoptosis and systemic autoimmunity: the dendritic cell connection. Eur J Histochem 2000; 44(3):229-36.
⁶⁵⁹ Rovere P, Vallinoto C, Bondanza A, Crosti M C, Rescigno M, Ricciardi-Castagnoli P, Rugarli C, Manfredi A A. Bystander apoptosis triggers dendritic cell maturation and antigen-presenting function. J. Immunol. 1998 Nov. 1; 161(9):4467-71.
⁶⁶⁰ Heath W R, Kurts C, Miller J F, Carbone F R. Cross-tolerance: a pathway for inducing tolerance to peripheral tissue antigens. J Exp Med. 1998 May 18; 187(10):1549-53.
⁶⁶¹ Sallusto F, Lanzavecchia A. Mobilizing dendritic cells for tolerance, priming, and chronic inflammation. J Exp Med. 1999 Feb. 15; 189(4):611-4.
⁶⁶² O'Brien B A, Harmon B V, Cameron D P, Allan D J. Beta-cell apoptosis is responsible for the development of IDDM in the multiple low-dose streptozotocin model. J Pathol. 1996 February; 178(2):176-81.
⁶⁶³ Lenzen S, Drinkgern J, Tiedge M. Low antioxidant enzyme gene expression in pancreatic islets compared with various other mouse tissues. Free Radic Biol Med. 1996; 20(3):463-6.
⁶⁶⁴ Tiedge M, Lortz S, Drinkgem J, Lenzen S. Relation between antioxidant enzyme gene expression and antioxidative defense status of insulin-producing cells. Diabetes 1997 November; 46(11):1733-42.
⁶⁶⁵ Harman A W, McKenna M, Adamson G M. Postnatal development of enzyme activities associated with protection against oxidative stress in the mouse. Biol Neonate. 1990; 57(3-4):187-93.
⁶⁶⁶ O'Brien B A, Harmon B V, Cameron D P, Allan D J. Nicotinamide prevents the development of diabetes in the cyclophosphamide-induced NOD mouse model by reducing beta-cell apoptosis. J Pathol. 2000 May; 191(1):86-92.
⁶⁶⁷ Hotta M, Tashiro F, Ikegami H, Niwa H, Ogihara T, Yodoi J, Miyazaki J. Pancreatic beta cell-specific expression of thioredoxin, an antioxidative and antiapoptotic protein, prevents autoimmune and streptozotocin-induced diabetes. J Exp Med; 1998 Oct. 19; 188(8):1445-51.
⁶⁶⁸ Kubisch H M, Wang J, Bray T M, Phillips J P. Targeted overexpression of Cu/Zn superoxide dismutase protects pancreatic beta-cells against oxidative stress. Diabetes 1997 October; 46(10):1563-6.
⁶⁶⁹ Kim J Y, Chi J K, Kim E J, Park S Y, Kim Y W, Lee S K. Inhibition of diabetes in non-obese diabetic mice by nicotinamide treatment for 5 weeks at the early age. J Korean Med Sci. 1997 August; 12(4):293-7.
⁶⁷⁰ Reddy S, Bibby N J, Elliott R B. Early nicotinamide treatment in the NOD mouse: effects on diabetes and insulitis suppression and autoantibody levels. Diabetes Res. 1990 October; 15(2):95-102.
⁶⁷¹ Beales P E, Williams A J, Albertini M C, Pozzilli P. Vitamin E delays diabetes onset in the non-obese diabetic mouse. Horm Metab Res. 1994 October; 26(10):450-2.
⁶⁷² Faust A, Burkart V, Ulrich H, Weischer C H, Kolb H. Effect of lipoic acid on cyclophosphamide-induced diabetes and insulitis in non-obese diabetic mice. Int J Immunopharmacol. 1994 January; 16(1):61-6.
⁶⁷³ Rabinovitch A, Suarez W L, Power R F. Lazaroid antioxidant decreases incidence of diabetes and insulitis in nonobese diabetic mice. J Lab Clin Med. 1993 April; 121(4):603-7.
⁶⁷⁴ Sevilla N, Kunz S, Holz A, Lewicki H, Homann D, Yamada H, Campbell K P, de La Torre J C, Oldstone M B. Immunosuppression and resultant viral persistence by specific viral targeting of dendritic cells. J Exp Med. 2000 Nov. 6; 192(9):1249-60.
⁶⁷⁵ Oldstone M B, Nerenberg M, Southern P, Price J, Lewicki H. Virus infection triggers insulin-dependent diabetes mellitus in a transgenic model: role of anti-self(virus) immune response. Cell. 1991 Apr. 19; 65(2):319-31.
⁶⁷⁶ Ohashi P S, Oehen S, Buerki K, Pircher H, Ohashi C T, Odermatt B, Malissen B, Zinkernagel R M, Hengartner H. Ablation of “tolerance” and induction of diabetes by virus infection in viral antigen transgenic mice. Cell. 1991 Apr. 19; 65(2):305-17.
⁶⁷⁷ Garza K M, Chan S M, Suri R, Nguyen L T, Odermatt B, Schoenberger S P, Ohashi P S. Role of antigen-presenting cells in mediating tolerance and autoimmunity. J Exp Med. 2000 Jun. 5; 191(11):2021-7.
⁶⁷⁸ Pradier O, Willems F, Abramowicz D, Schandene L, de Boer M, Thielemans K, Capel P, Goldman M. CD40 engagement induces monocyte procoagulant activity through an interleukin-10 resistant pathway. Eur J. Immunol. 1996 December; 26(12):3048-54.
⁶⁷⁹Moodycliffe A M, Shreedhar V, Ullrich S E, Walterscheid J, Bucana C, Kripke M L, Flores-Romo L. CD40-CD40 ligand interactions in vivo regulate migration of antigen-bearing dendritic cells from the skin to draining lymph nodes. J Exp Med. 2000 Jun. 5; 191(11):2011-20.
⁶⁸⁰ von Herrath M, Holz A. Pathological changes in the islet milieu precede infiltration of islets and destruction of beta-cells by autoreactive lymphocytes in a transgenic model of virus-induced IDDM. J Autoimmun. 1997 June; 10(3):231-8.
⁶⁸¹ Ehl S, Hombach J, Aichele P, Rulicke T, Odermatt B, Hengartner H, Zinkernagel R, Pircher H. Viral and bacterial infections interfere with peripheral tolerance induction and activate CD8+ T-cells to cause immunopathology. J Exp Med. 1998 Mar. 2; 187(5):763-74.
⁶⁸² Ilowite N T. Premature atherosclerosis in systemic lupus erythematosus. J Rheumatol. 2000 April; 27 Suppl 58:15-9.
⁶⁸³ Urowitz M, Gladman D, Bruce I. Atherosclerosis and Systemic Lupus Erythematosus. Curr Rheumatol Rep 2000 February; 2(1):19-23
⁶⁸⁴ Dobado-Berrios P M, Lopez-Pedrera C, Velasco F, Aguirre M A, Torres A, Cuadrado M J. Increased levels of tissue factor mRNA in mononuclear blood cells of patients with primary antiphospholipid syndrome. Thromb Haemost 1999 December; 82(6):1578-82.
⁶⁸⁵ Ludewig B, Odermatt B, Landmann S, Hengartner H, Zinkernagel R M. Dendritic cells induce autoimmune diabetes and maintain disease via de novo formation of local lymphoid tissue. J Exp Med. 1998 Oct. 19; 188(8):1493-501.
⁶⁸⁶ Fearnley D B, Whyte L F, Carnoutsos S A, Cook A H, Hart D N. Monitoring human blood dendritic cell numbers in normal individuals and in stem cell transplantation. Blood. 1999 Jan. 15; 93(2):728-36.
⁶⁸⁷ Barratt-Boyes S M, Zimmer M I, Harshyne L A, Meyer E M, Watkins S C, Capuano S, Murphey-Corb M, Falo L D, Donnenberg A D. Maturation and trafficking of monocyte-derived dendritic cells in monkeys: implications for dendritic cell-based vaccines. J. Immunol. 2000 Mar. 1; 164(5):2487-95.
⁶⁸⁸ Hermans I F, Ritchie D S, Yang J, Roberts J M, Ronchese F. CD8+ T-cell-dependent elimination of dendritic cells in vivo limits the induction of antitumor immunity. J. Immunol. 2000 Mar. 15; 164(6):3095-101.
⁶⁸⁹ Ludewig B, Ochsenbein A F, Odermatt B, Paul in D, Hengartner H, Zinkernagel R M. Immunotherapy with dendritic cells directed against tumor antigens shared with normal host cells results in severe autoimmune disease. J Exp Med. 2000 Mar. 6; 191(5):795-804.
⁶⁹⁰ Roskrow M A, Dilloo D, Suzuki N, Zhong W, Rooney C M, Brenner M K. Autoimmune disease induced by dendritic cell immunization against leukemia. Leuk Res 1999 June; 23(6):549-57.
⁶⁹¹ Lipton H L, Pritchard A E, Calenoff M A. Attenuation of neurovirulence of Theiler's murine encephalomyelitis virus strain GDVII is not sufficient to establish persistence in the central nervous system. J Gen Virol. 1998 May; 79 (Pt 5):1001-4.
⁶⁹² Tsunoda I, Kurtz C I, Fujinami R S. Apoptosis in acute and chronic-central nervous system disease induced by Theiler's murine encephalomyelitis virus. Virology. 1997 Feb. 17; 228(2):388-93.
⁶⁹³ Ha-Lee Y M, Dillon K, Kosaras B, Sidman R, Revell P, Fujinami R, Chow M. Mode of spread to and within the central nervous system after oral infection of neonatal mice with the DA strain of Theiler's murine encephalomyelitis virus. J. Virol. 1995 November; 69(11):7354-61.
⁶⁹⁴ Lipton H L, Twaddle G, Jelachich M L. The predominant virus antigen burden is present in macrophages in Theiler's murine encephalomyelitis virus-induced demyelinating disease. J. Virol. 1995 April; 69(4):2525-33.
⁶⁹⁵ Miller S D, Vanderlugt C L, Begolka W S, Pao W, Yauch R L, Neville K L, Katz-Levy Y, Carrizosa A, Kim B S. Persistent infection with Theiler's virus leads to CNS autoimmunity via epitope spreading. Nat Med. 1997 October; 3(10):1133-6.
⁶⁹⁶ Vermaelen K Y, Carro-Muino I, Lambrecht B N, Pauwels R A. Specific Migratory Dendritic Cells Rapidly Transport Antigen from the Airways to the Thoracic Lymph Nodes. J Exp Med 2001 Jan. 1; 193(1):51-60.
⁶⁹⁷ Lambrecht B N, Pauwels R A, Fazekas De St Groth B. Induction of rapid T-cell activation, division, and recirculation by intratracheal injection of dendritic cells in a TCR transgenic model. J. Immunol. 2000 Mar. 15; 164(6):2937-46.
⁶⁹⁸ Lambrecht B N, De Veerman M, Coyle A J, Gutierrez-Ramos J C, Thielemans K, Pauwels R A. Myeloid dendritic cells induce Th2 responses to inhaled antigen, leading to eosinophilic airway inflammation. J Clin Invest. 2000 August; 106(4):551-9.
⁶⁹⁹ Bertorelli G. Bocchino V, Zhou X, Zanini A, Bernini M V, Damia R, Di Comite V, Grima P, Olivieri D. Dendritic cell number is related to IL-4 expression in the airways of atopic asthmatic subjects. Allergy. 2000 May; 55(5):449-54.
⁷⁰⁰ Lambrecht B N, Salomon B, Klatzmann D, Pauwels R A. Dendritic cells are required for the development of chronic eosinophilic airway inflammation in response to inhaled antigen in sensitized mice. J. Immunol. 1998 Apr. 15; 160(8):4090-7.
⁷⁰¹ Mathur M, Herrmann K, Qin Y, Gulmen F, Li X, Krimins R, Weinstock J, Elliott D, Bluestone J A, Padrid P. CD28 interactions with either CD80 or CD86 are sufficient to induce allergic airway inflammation in mice. Am J Respir Cell Mol. Biol. 1999 October; 21(4):498-509.
⁷⁰² Haczku A, Takeda K, Redai I, Hamelmann E, Cieslewicz G, Joetham A, Loader J, Lee J J, Irvin C, Gelfand E W. Anti-CD86 (B7.2) treatment abolishes allergic airway hyperresponsiveness in mice. Am J Respir Crit Care Med. 1999 May; 159(5 Pt 1): 1638-43.
⁷⁰³ Padrid P A, Mathur M, Li X, Herrmann K, Qin Y, Cattamanchi A, Weinstock J, Elliott D, Sperling A I, Bluestone J A. CTLA41g inhibits airway eosinophilia and hyperresponsiveness by regulating the development of Th1/Th2 subsets in a murine model of asthma. Am J Respir Cell Mol. Biol. 1998 April; 18(4):453-62.
⁷⁰⁴ Keane-Myers A M, Gause W C, Finkelman F D, Xhou X D, Wills-Karp M. Development of murine allergic asthma is dependent upon B7-2 costimulation. J. Immunol. 1998 Jan. 15; 160(2):1036-43.
⁷⁰⁵ Balsa A, Dixey J, Sansom D M, Maddison P J, Hall N D. Differential expression of the costimulatory molecules B7.1 (CD80) and B7.2 (CD86) in rheumatoid synovial tissue. Br J Rheumatol 1996 January; 35(1):33-7.
⁷⁰⁶ Liu M F, Kohsaka H, Sakurai H, Azuma M, Okumura K, Saito I, Miyasaka N. The presence of costimulatory molecules CD86 and CD28 in rheumatoid arthritis synovium. Arthritis Rheum. 1996 January; 39(1):110-4.
⁷⁰⁷ Watanabe H, Inaba M, Adachi Y, Sugiura K, Hisha H, Iguchi T, Ito T, Yasumizu R, Inaba K, Yamashita T, Ikehara S. Experimental autoimmune thyroiditis induced by thyroglobulin-pulsed dendritic cells. Autoimmunity 1999; 31(4):273-82.
⁷⁰⁸ Tandon N, Metcalfe R A, Barnett D, Weetman A P. Expression of the costimulatory molecule B7/BB1 in autoimmune thyroid disease. Q J. Med. 1994 April; 87(4):2316.
⁷⁰⁹ Signore A, Chianelli M, Parisella M G, Capriotti &, Giacalone P. Di Leve G. Barone R. In vivo imaging of insulitis in autoimmune diabetes. J Endocrinol Invest. 1999 February; 22(2):151-8.
⁷¹⁰ Foulis A K, McGill M, Farquharson M A. Insulitis in type I (insulin-dependent) diabetes mellitus in man—macrophages, lymphocytes, and interferon-gamma containing cells. J Pathol. 1991 October; 165(2):97-103.
⁷¹¹ Foulis A K, Stewart J A. The pancreas in recent-onset type I (insulin-dependent) diabetes mellitus: insulin content of islets, insulitis and associated changes in the exocrine acinar tissue. Diabetologia. 1984 June; 26(6):456-61.
⁷¹² Bitsch A, Bruck W. Differentiation of multiple sclerosis subtypes: implications for treatment. CNS Drugs. 2002; 16(6):405-18. Review.
⁷¹³ Brown K A. Factors modifying the migration of lymphocytes across the blood-brain barrier. Int Immunopharmacol. 2001 November; 1(12):2043-62. Review.
⁷¹⁴ Pouly S, Antel J P. Multiple sclerosis and central nervous system demyelination. J Autoimmun. 1999 November; 13(3):297-306. Review.
⁷¹⁵ Bata-Csorgo Z, Hammerberg C, Voorhees J J, Cooper K D. Intralesional T-lymphocyte activation as a mediator of psoriatic epidermal hyperplasia. J Invest Dermatol. 1995 July; 105(1 Suppl): 89S-94S. Review.
⁷¹⁶ Baadsgaard O, Fisher G, Voorhees J J, Cooper K D. The role of the immune system in the pathogenesis of psoriasis. J Invest Dermatol. 1990 November; 95(5):32S-34S. Review.
⁷¹⁷ Hoffman R W. T-cells in the pathogenesis of systemic lupus erythematosus. Front Biosci. 2001 Oct. 1; 6:D1369-78. Review.
⁷¹⁸ Chan O T, Madaio M P, Shlomchik M J. The central and multiple roles of B cells in lupus pathogenesis. Immunol Rev. 1999 June; 169:107-21. Review.
⁷¹⁹ Trautmann A, Schmid-Grendelmeier P, Kruger K, Crameri R, Akdis M, Akkaya A, Brocker E B, Blaser K, Akdis C A. T-cells and eosinophils cooperate in the induction of bronchial epithelial cell apoptosis in asthma. J Allergy Clin Immunol. 2002 February; 109(2):329-37.
⁷²⁰ Poston R N, Chanez P, Lacoste J Y, Litchfield T, Lee T H, Bousquet J. Immunohistochemical characterization of the cellular infiltration in asthmatic bronchi. Am Rev Respir Dis. 1992 April; 145(4 Pt 1):918-21.
⁷²¹ Strober S, Holoshitz J. Mechanisms of immune injury in rheumatoid arthritis: evidence for the involvement of T-cells and heat-shock protein. Immunol Rev. 1990 Dec; 118:233-55. Review.
⁷²² Stassi G, Zeuner A, Di Liberto D, Todaro M, Ricci-Vitiani L, De Maria R. Fas-FasL in Hashimoto's thyroiditis. J Clin Immunol. 2001 January; 21(1):19-23. Review.
⁷²³ Eguchi K. Apoptosis in autoimmune diseases. Intern Med. 2001 April; 40(4):275-84. Review.
⁷²⁴ De Simone R, Giampaolo A, Giometto B, Gallo P, Levi G, Peschle C, Aloisi F. The costimulatory molecule B7 is expressed on human microglia in culture and in multiple sclerosis acute lesions. J Neuropathol Exp Neurol. 1995 March; 54(2): 175-87.
⁷²⁵ Windhagen A, Newcombe J, Dangond F, Strand C, Woodroofe M N, Cuzner M L, Hafler D A. Expression of costimulatory molecules B7-1 (CD80), B7-2 (CD86), and interleukin 12 cytokine in multiple sclerosis lesions. J Exp Med. 1995 Dec. 11; 82(6):1985-96.
⁷²⁶ Ohki O, Yokozeki H, Katayama I, Umeda T, Azuma M, Okumura K, Nishioka K. Functional CD86 (B7-2/B70) is predominantly expressed on Langerhans cells in atopic dermatitis. Br J Dermatol 1997 June; 136(6):838-45.
⁷²⁷ Agea E, Forenza N, Piattoni S, Russano A, Monaco A, Flenghi L, Bistoni O, Gillies D A, Azuma M, Bertotto A, Spinozzi F. Expression of B7 co-stimulatory molecules and CD1a antigen by alveolar macrophages in allergic bronchial asthma. Clin Exp Allergy. 1998 November; 28(11):1359-67.
⁷²⁸ Balbo P, Silvestri M, Rossi G A, Crimi E, Burastero S E. Differential role of CD80 and CD86 on alveolar macrophages in the presentation of allergen to T lymphocytes in asthma. Clin Exp Allergy. 2001 April; 31(4):625-36.
⁷²⁹ Burastero S E, Magnani Z, Confetti C, Abbruzzese L, Oddera S, Balbo P, Rossi G A, Crimi E. Increased expression of the CD80 accessory molecule by alveolar macrophages in asthmatic subjects and its functional involvement in allergen presentation to autologous TH2 lymphocytes. Allergy Clin Immunol. 1999 June; 103(6):1136-42.
⁷³⁰ Hofer M F, Jirapongsananuruk O, Trumble A E, Leung D Y. Upregulation of B7.2, but not B7.1, on B cells from patients with allergic asthma. J Allergy Clin Immunol. 1998 January; 101(1 Pt 1):96-102.
⁷³¹ Rogler G, Hausmann M, Spottl T, Vogl D, Aschenbrenner E, Andus T, Falk W, Scholmerich J, Gross V. T-cell co-stimulatory molecules are upregulated on intestinal macrophages from inflammatory bowel disease mucosa. Eur J Gastroenterol Hepatol. 1999 October; 11(10): 1105-11.
⁷³² Rugtveit J, Bakka A, Brandtzaeg P. Differential distribution of B7.1 (CD80) and B7.2 (CD86) costimulatory molecules on mucosal macrophage subsets in human inflammatory bowel disease (IBD). Clin Exp Immunol. 1997 October; 110(1):104-13.
⁷³³ Hara J, Ohtani H, Matsumoto T, Nakamura S, Kitano A, Arakawa T, Nagura H, Kobayashi K. Expression of costimulatory molecules B7-1 and B7-2 in macrophages and granulomas of Crohn's disease: demonstration of cell-to-cell contact with T lymphocytes. Lab Invest. 1991 August; 77(2):175-84.
⁷³⁴ Liu Z X, Hiwatashi N, Noguchi M, Toyota T. Increased expression of costimulatory molecules on peripheral blood monocytes in patients with Crohn's disease. Scand J Gastroenterol. 1997 December; 32(12):1241-6.
⁷³⁵ Shimoyama Y, Nagafuchi H, Suzuki N, Ochi T, Sakane T. Synovium infiltrating T-cells induce excessive synovial cell function through CD28/B7 pathway in patients with rheumatoid arthritis. J Rheumatol. 1999 October; 26(10):2094-101.
⁷³⁶ Thomas R, Quinn C. Functional differentiation of dendritic cells in rheumatoid arthritis: role of CD86 in the synovium. J. Immunol. 1996 Apr. 15; 156(8):3074-86.
⁷³⁷ Denfeld R W, Kind P, Sontheimer R D, Schopf E, Simon J C. In situ expression of B7 and CD28 receptor families in skin lesions of patients with lupus erythematosus. Arthritis Rheum. 1997 May; 40(5):814-21.
⁷³⁸ Simon J C, Dietrich A, Mielke V, Wuttig C, Vanscheidt W, Linsley P S, Schopf E, Sterry W. Expression of the B7/BB1 activation antigen and its ligand CD28 in T-cell-mediated skin diseases. J Invest Dermatol. 1994 October; 103(4):539-43.
⁷³⁹ Boven L A, Montagne L, Nottet H S, De Groot C J. Macrophage inflammatory protein-1alpha (MIP-1 alpha), MIP-1beta, and RANTES mRNA semiquantification and protein expression in active demyelinating multiple sclerosis (MS) lesions. Clin Exp Immunol. 2000 November; 122(2):257-63.
⁷⁴⁰ Alam R, York J, Boyars M, Stafford S, Grant J A, Lee J, Forsythe P, Sim T, Ida N. Increased MCP-1, RANTES, and MIP-t alpha in bronchoalveolar lavage fluid of allergic asthmatic patients. Am J Respir Crit Care Med. 1996 April; 153(4 Pt 1): 1398404.
⁷⁴¹ Hsieh K H, Chou C C, Chiang B L. Immunotherapy suppresses the production of monocyte chemotactic and activating factor and augments the production of IL-8 in children with asthma. J Allergy Clin Immunol. 1996 September; 98(3):580-7.
⁷⁴² Holgate S T, Bodey K S, Janezic A, Frew A J, Kaplan A P, Teran L M. Release of RANTES, MIP-1 alpha, and MCP-1 into asthmatic airways following endobronchial allergen challenge. Am J Respir Crit Care Med. 1997 November; 156(5): 1377-83.
⁷⁴³ Banks C, Bateman A, Payne R, Johnson P, Sheron N. Chemokine expression in IBD. Mucosal chemokine expression is unselectively increased in both ulcerative colitis and Crohn's disease. J Pathol. 2003 January; 199(1):28-35.
⁷⁴⁴ Uguccioni M, Gionchetti P, Robbiani D F, Rizzello F, Peruzzo S, Campieri M, Baggiolini M. Increased expression of IP-10, IL-8, MCP-1, and MCP-3 in ulcerative colitis. Am J Pathol. 1999 August; 155(2):331-6.
⁷⁴⁵ Vainer B, Nielsen O H, Horn T. Expression of E-selectin, sialyl Lewis X, and macrophage inflammatory protein-1alpha by colonic epithelial cells in ulcerative colitis. Dig Dis Sci. 1998 March; 43(3):596-608.
⁷⁴⁶ Hatano Y, Katagiri K, Takayasu S. Increased levels in vivo of mRNAs for IL-8 and macrophage inflammatory protein-1 alpha (MIP-1 alpha), but not of RANTES mRNA in peripheral blood mononuclear cells of patients with atopic dermatitis (AD). Clin Exp Immunol. 1999 August; 117(2):237-43.
⁷⁴⁷ Katrib A, Tak P P, Bertouch J V, Cuello C, McNeil H P, Smeets T J, Kraan M C, Youssef P P. Expression of chemokines and matrix metalloproteinases in early rheumatoid arthritis. Rheumatology (Oxford). 2001 September; 40(9):988-94.
⁷⁴⁸ Volin M V, Shah M R, Tokuhira M, Haines G K, Woods J M, Koch A E. RANTES expression and contribution to monocyte chemotaxis in arthritis. Clin Immunol Immunopathol 1998 October; 89(1):44-53.
⁷⁴⁹ al-Mughales J, Blyth T H, Hunter J A, Wilkinson P C. The chemoattractant activity of rheumatoid synovial fluid for human lymphocytes is due to multiple cytokines. Clin Exp Immunol. 1996 November; 106(2):230-6.
⁷⁵⁰ Hosaka S, Akahoshi T, Wada C, Kondo H. Expression of the chemokine superfamily in rheumatoid arthritis. Clin Exp Immunol. 1994 September; 97(3):451-7.
⁷⁵¹ Cuello C, Palladinetti P, Tedla N, Di Girolamo N, Lloyd A R, McCluskey P J, Wakefield D. Chemokine expression and leucocyte infiltration in Sjogren's syndrome. Br J Rheumatol. 1998 Ju1; 37(7):779-83.
⁷⁵² Katrib A, Smith M D, Ahem M J, Slavotinek J, Stafford L, Cuello C, Bertouch J V, McNeil H P, Youssef P P. Reduced chemokine and matrix metalloproteinase expression in patients with rheumatoid arthritis achieving remission. J Rheumatol. 2003 January; 30(1):10-21.
⁷⁵³ Matteucci E, Giampietro O. Oxidative stress in families of type 1 diabetic patients. Diabetes Care. 2000 August; 23(8):1182-6.
⁷⁵⁴ Serban V, Dabelea D, Deutsch G, Pataki C, Dan I. Apolipoprotein (a) concentrations in type I (insulin-dependent) diabetes mellitus. Rom J Intern Med. 1995 January-June; 33(1-2):77-83.
⁷⁵⁵ Busso N, Dudler J, Salvi R, Peclat V, Lenain V, Marcovina S, Darioli R, Nicod P, So A K, Mooser V. Plasma apolipoprotein(a) co-deposits with fibrin in inflammatory arthritic joints. Am J Pathol. 2001 October; 159(4):1445-53.
⁷⁵⁶ Asanuma Y, Kawai S, Aoshima H, Kaburaki J, Mizushima Y. Serum lipoprotein(a) and apolipoprotein(a) phenotypes in patients with rheumatoid arthritis. Arthritis Rheum. 1999 March; 42(3):443-7.
⁷⁵⁷ Lee Y H, Choi S J, Ji J D, Seo H S, Song G G. Lipoprotein(a) and lipids in relation to inflammation in rheumatoid arthritis. Clin Rheumatol. 2000; 19(4):324-5.
⁷⁵⁸ Park Y B, Lee S K, Lee W K, Suh C H, Lee C W, Lee C H, Song C H, Lee J. Lipid profiles in untreated patients with rheumatoid arthritis. J Rheumatol. 1999 August; 26(8): 1701-4.
⁷⁵⁹ Sari R A, Polat M F, Taysi S, Bakan E, Capoglu I. Serum Lipoprotein(a) Level and its Clinical Significance in Patients with Systemic Lupus Erythematosus. Clin Rheumatol. 2002 November; 21(6):520-4.
⁷⁶⁰ Yamazaki M, Asakura H, Jokaji H, Saito M, Uotani C, Kumabashiri I, Morishita E, Aoshima K, Ikeda T, Matsuda T. Plasma levels of lipoprotein(a) are elevated in patients with the antiphospholipid antibody syndrome. Thromb Haemost. 1994 April; 71(4):424-7.
⁷⁶¹ Atsumi T, Khamashta M A, Andujar C, Leandro M J, Amengual O, Ames P R, Hughes G R. Elevated plasma lipoprotein(a) level and its association with impaired fibrinolysis in patients with antiphospholipid syndrome. J Rheumatol. 1998 January; 25(1):69-73.
⁷⁶² Tzotzas T, Krassas G E, Konstantinidis T, Bougoulia M. Changes in lipoprotein(a) levels in overt and subclinical hypothyroidism before and during treatment. Thyroid. 2000 September; 10(9):803-8.
⁷⁶³ Kung A W, Pang R W, Janus E D. Elevated serum lipoprotein(a) in subclinical hypothyroidism. Clin Endocrinol (Oxf). 1995 October; 43(4):445-9.
⁷⁶⁴ Klausen I C, Nielsen F E, Hegedus L, Gerdes L U, Charles P, Faergeman O. Treatment of hypothyroidism decreases low-density lipoproteins but not lipoprotein(a). Metabolism. 1992 August; 41(8):911-4.
⁷⁶⁵ Engler H, Riesen W F. Effect of thyroid function on concentrations of lipoprotein(a). Clin Chem. 1993 December; 39(12):2466-9.
⁷⁶⁶ de Bruin T W, van Barlingen H, van Linde-Sibenius Trip M, van Vuurst de Vries A R, Akveld M J, Erkelens D W. Lipoprotein(a) and apolipoprotein B plasma concentrations in hypothyroid, euthyroid, and hyperthyroid subjects. J Clin Endocrinol Metab. 1993 January; 76(1):121-6.
⁷⁶⁷ van Bodegraven A A, Meuwissen S G. Lipoprotein (a), thrombophilia and inflammatory bowel disease. Eur J Gastroenterol Hepatol. 2001 December; 13(12):1407-9. Review.
⁷⁶⁸ Koutroubakis I E, Malliaraki N, Vardas E, Ganotakis E, Margioris A N, Manousos O N, Kouroumalis E A. Increased levels of lipoprotein (a) in Crohn's disease: a relation to thrombosis? Eur J Gastroenterol Hepatol. 2001 December; 13(12):1415-9.
⁷⁶⁹ Kawabata S, Katagiri S, Negoro H, Nogami A, Yabuuchi I, Gomi M, Arima R, Tarui S. Elevated serum lipoprotein (a) levels associated with ulcerative colitis in a young Japanese patient. Intern Med. 1997 June; 36(6):389-91.
⁷⁷⁰ Uyanik B S, Ari Z, Onur E, Gunduz K, Tanulku S, Durkan K. Serum lipids and apolipoproteins in patients with psoriasis. Clin Chem Lab Med. 2002 January; 40(1):65-8.
⁷⁷¹ Rocha-Pereira P, Santos-Silva A, Rebelo I, Figueiredo A, Quintanilha A, Teixeira F. Dislipidemia and oxidative stress in mild and in severe psoriasis as a risk for cardiovascular disease. Clin Chim Acta. 2001 January; 303(1-2):33-9.
⁷⁷² Camp R D. The variation of serum lipoprotein (a) level with disease activity in psoriasis. Br J. Dermatol. 1999 March; 140(3):566-7.
⁷⁷³ Cimsit G, Orem A, Deger O, Alpay K, Kiran E, Orem C. The variation of serum lipoprotein (a) level with disease activity in psoriasis. Br J Dermatol 1998 May; 138(5):917-9.
⁷⁷⁴ Seckin D, Tokgozoglu L, Akkaya S. Are lipoprotein profile and lipoprotein (a) levels altered in men with psoriasis? J Am Acad Dermatol. 1994 September; 31(3 Pt 1):445-9.
⁷⁷⁵ Kozawa O, Hatakeyama D, Yoshida M, Kamiya Y, Kondo C, Matsuno H, Uematsu T. Activation of p44/p42 mitogen-activated protein kinase limits triiodothyronine-stimulated alkaline phosphatase activity in osteoblasts. Biochem Biophys Res Commun. 2001 Sep. 7; 286(5):1140-3.
⁷⁷⁶ Lin H Y, Davis F B, Gordinier J K, Martino L J, Davis P J. Thyroid hormone induces activation of mitogen-activated protein kinase incultured cells. Am J. Physiol. 1999 May; 276(5 Pt 1):C1014-24.
⁷⁷⁷ Hoppichler F, Sandlolzer C, Moncayo R, Utermann G, Kraft H G. Thyroid hormone (fT4) decreases lipoprotein(a) plasma levels. Atherosclerosis. 1995 May; 115(1):65-71.
⁷⁷⁸ Kung A W, Pang R W, Lauder I, Lam K S, Janus E D. Changes in serum lipoprotein(a) and lipids during treatment of hyperthyroidism. Clin Chem. 1995 February; 41(2):226-31.
⁷⁷⁹ Loots M A, Lamme E N, Zeegelaar J, Mekkes J R, Bos J D, Middelkoop E. Differences in cellular infiltrate and extracellular matrix of chronic diabetic and venous ulcers versus acute wounds. J Invest Dermatol. 1998 November; 111(5):850-7.
⁷⁸⁰ Spirin K S, Saghizadeh M, Lewin S L, Zardi L, Kenney M C, Ljubimov A V. Basement membrane and growth factor gene expression in normal and diabetic human retinas. Curr Eye Res. 1999 June; 18(6):490-9.
⁷⁸¹ Amin K, Ludviksdottir D, Janson C, Nettelbladt O, Bjornsson E, Roomans G M, Boman G, Seveus L, Venge P. Inflammation and structural changes in the airways of patients with atopic and nonatopic asthma. BHR Group. Am J Respir Crit Care Med. 2000 December; 162(6):2295-301.
⁷⁸² Karjalainen E M, Laitinen A, Sue-Chu M, Altraja A, Bjermer L, Laitinen L A. Evidence of airway inflammation and remodeling in ski athletes with and without bronchial hyperresponsiveness to methacholine. Am J Respir Crit Care Med. 2000 June; 161(6):2086-91.
⁷⁸³ Laitinen A, Altraja A, Kampe M, Linden M, Virtanen I, Laitinen L A. Tenascin is increased in airway basement membrane of asthmatics and decreased by an inhaled steroid. Am J Respir Crit Care Med. 1997 September; 156(3 Pt 1):951-8.
⁷⁸⁴ Laitinen L A, Laitinen A, Altraja A, Virtanen I, Kampe M, Simonsson B G, Karlsson S E, Hakansson L, Venge P, Sillastu H. Bronchial, biopsy findings in intermittent or “early” asthma. J Allergy Clin Immunol. 1996 November; 98(5 Pt 2)S3-6; discussion S33-40.
⁷⁸⁵ Back W, Heubner C, Winter J, Bleyl U. Expression of tenascin in lymphocytic autoimmune thyroiditis. J Clin Pathol. 1997 October; 50(10):863-6.
⁷⁸⁶ Amin K, Ludviksdottir D, Janson C, Nettelbladt O, Gudbjornsson B, Valtysdottir S, Bjornsson E, Roomans G M, Boman G, Seveus L, Venge P; BHR-Group. Bronchial hyper-responsiveness. Inflammation and structural changes in the airways of patients with primary Sjogren's syndrome. Respir Med. 2001 November; 95(11):904-10.
⁷⁸⁷ Geboes K, El-Zine M Y, Dalle I, El-Haddad S, Rutgeerts P, Van Eyken P. Tenascin and strictures in inflammatory bowel disease: an immunohistochemical study. Int J Surg Pathol. 2001 October; 9(4):281-6.
⁷⁸⁸ Riedl S, Tandara A, Reinshagen M, Hinz U, Faissner A, Bodenmuller H, Buhr H J, Herfarth C, Moller P. Serum tenascin-C is an indicator of inflammatory bowel disease activity. Int J Colorectal Dis. 2001 September; 16(5):285-91.
⁷⁸⁹ Riedi S, Kadmon M, Tandara A, Hinz U, Moller P, Herfarth C, Faissner A. Mucosal tenascin C content in inflammatory and neoplastic diseases of the large bowel. Dis Colon Rectum. 1998 January; 41(1):86-92.
⁷⁹⁰ Riedl S, Kadmon M, Tandara A, Hinz U, Moller P, Faissner A. Tenascin-C tissue concentration in inflammatory and neoplastic diseases of the colon mucosa. Anticancer Res. 1997 July-August; 17(4B):3165-6.
⁷⁹¹ Latijnhouwers M A, Bergers M, Kuijpers A L, van der Vleuten C J, Dijkman H, van de Kerkhof P C, Schalkwijk J. Tenascin-C is not a useful marker for disease activity in psoriasis. Acta Derm Venereol. 1998 September; 78(5):331-4.
⁷⁹² Schalkwijk J, Van Vlijmen I, Oosterling B, Perret C, Koopman R, Van den Bom J, Mackie E J. Tenascin expression in hyperproliferative skin diseases. Br J. Dermatol. 1991 January; 124(1):13-20.
⁷⁹³ Salter D M. Tenascin is increased in cartilage and synovium from arthritic knees. Br J Rheumatol. 1993 September; 32(9):780-6.
⁷⁹⁴ Chevalier X, Groult N, Larget-Piet B, Zardi L, Hornebeck W. Tenascin distribution in articular cartilage from normal subjects and from patients with osteoarthritis and rheumatoid arthritis. Arthritis Rheum. 1994 July; 37(7):1013-22.
⁷⁹⁵ Li X H, Li T L, Yang Z, Liu Z Y, Wei Y D, Jin S X, Hong C, Qin R L, Li Y Q, Dorman J S, Laporte R E, Wang K A. A nine-year prospective study on the incidence of childhood type 1 diabetes mellitus in China. Biomed Environ Sci. 2000 December; 13(4):263-70.
⁷⁹⁶ Huen K F, Low L C, Wong G W, Tse W W, Yu A C, Lam Y Y, Cheung P C, Wong L M, Yeung W K, But B W, Cheung P T, Kwan E Y, Karlberg J P, Lee C. Epidemiology of diabetes mellitus in children in Hong Kong: the Hong Kong childhood diabetes register. J Pediatr Endocrinol Metab 2000 March; 13(3):297-302.
⁷⁹⁷ Karjalainen J, Salmela P, Ilonen J, Surcel H M, Knip M. A comparison of childhood and adult type I diabetes mellitus. N Engl J. Med. 1989 Apr. 6; 320(14):881-6.
⁷⁹⁸ Green A, Andersen P K. Epidemiological studies of diabetes mellitus in Denmark: 3. Clinical characteristics and incidence of diabetes among males aged 0 to 19 years. Diabetologia. 1983 September; 25(3):226-30.
⁷⁹⁹ Swanbeck G, Inerot A, Martinsson T, Wahlstrom J, Enerback C, Enlund F, Yhr M. Age at onset and different types of psoriasis. Br J. Dermatol. 1995 November; 133(5):768-73.
⁸⁰⁰ Khanna K V, Markham R B. A perspective on cellular immunity in the elderly. Clin Infect Dis 1999 April; 28(4):710-3.
⁸⁰¹ Ginaldi L, De Martinis M, D'Ostilio A, Marini L, Loreto M F, Martorelli V, Quaglino D. The immune system in the elderly: 11. Specific cellular immunity. Immunol Res 1999; 20(2):109-15.
⁸⁰² Liedtke W, Opalka B, Zimmermann C W, Lignitz E. Age distribution of latent herpes simplex virus I and varicella-zoster virus genome in human nervous tissue. J Neurol Sci. 1993 May; 116(1):6-11.
⁸⁰³ Beer W E, Smith A E, Kassab J Y, Smith P H, Rowland Payne C M. Concomitance of psoriasis and atopic dermatitis. Dermatology. 1992; 184(4):265-70.
⁸⁰⁴ Asadullah K, Prosch S, Audring H, Buttnerova I, Volk H D, Sterry W, Docke W D. A high prevalence of cytomegalovirus antigenaemia in patients with moderate to severe chronic plaque psoriasis: an association with systemic tumour necrosis factor alpha overexpression. Br J. Dermatol. 1999 July; 141(1):94-102.
⁸⁰⁵ Steigleder G K, Rasokat H, Wemmer U. [Psoriasis in HTLV-III-induced immunologic defect—status of cellular immunity and immunohistologic findings]. Z Hautkr 1986 Dec. 1; 61(23):1671-8. [Article in German]
⁸⁰⁶ Mallon E. Retroviruses and psoriasis. Curr Opin Infect Dis. 2000 April; 13(2):103-107.
⁸⁰⁷ Montazeri A, Kanitakis J, Bazex J. Psoriasis and HIV infection. Int J. Dermatol. 1996 July; 35(7):475-9.
⁸⁰⁸ Fischer T, Schworer H, Vente C, Reich K, Ramadori G. Clinical improvement of HIV-associated psoriasis parallels a decrease of HIV viral load induced by effective antiretroviral therapy. AIDS. 1999 Apr. 1; 13(5):628-9.
⁸⁰⁹ Olsson J, Poles M, Spetz A L, Elliott J, Hultin L, Giorgi J, Andersson J, Anton P. Human immunodeficiency virus type I infection is associated with significant mucosal inflammation characterized by increased expression of CCR5, CXCR4, and beta-chemokines. J Infect Dis. 2000 December; 182(6):1625-35.
⁸¹⁰ Roivainen M, Rasilainen S, Ylipaasto P, Nissinen R, Ustinov J, Bouwens L, Eizirik D L, Hovi T, Otonkoski T. Mechanisms of coxsackievirus-induced damage to human pancreatic beta-cells. J Clin Endocrinol Metab 2000 January; 85(1):432-40.
⁸¹¹ Andreoletti L, Hober D, Hober-Vandenberghe C, Fajardy I, Belaich S, Lambert V, Vantyghem M C, Lefebvre J, Wattre P. Coxsackie B virus infection and beta cell autoantibodies in newly diagnosed IDDM adult patients. Clin Diagn Virol. 1998 April; 9(2-3):125-33.
⁸¹² Andreoletti L, Hober D, Hober-Vandenberghe C, Belaich S, Vantyghem M C, Lefebvre J, Wattre P. Detection of coxsackie B virus RNA sequences in whole blood samples from adult patients at the onset of type I diabetes mellitus. J Med Virol. 1997 June; 52(2): 121-7.
⁸¹³ Frisk G, Diderholm H. Antibody responses to different strains of coxsackie B4 virus in patients with newly diagnosed type I diabetes mellitus or aseptic meningitis. J. Infect. 1997 May; 34(3):205-10.
⁸¹⁴ Clements G B, Galbraith D N, Taylor K W. Coxsackie B virus infection and onset of childhood diabetes. Lancet. 1995 Jul. 22; 346(8969):221-3.
⁸¹⁵ Amin K, Ekberg-Jansson A, Lofdahl C G, Venge P. Relation between inflammatory cells and structural changes in the lungs of asymptomatic and never smokers: a biopsy study. Thorax. 2003 February; 58(2):135-42.
⁸¹⁶ Abrams J R, Kelley S L, Hayes E, Kikuchi T, Brown M J, Kang S, Lebwohl M G, Guzzo C A, Jegasothy B V, Linsley P S, Krueger J G. Blockade of T lymphocyte costimulation with cytotoxic T lymphocyte-associated antigen 4-immunoglobulin (CTLA41g) reverses the cellular pathology of psoriatic plaques, including the activation of keratinocytes, dendritic cells, and endothelial cells. J Exp Med. 2000 Sep. 4; 192(5):681-94.
⁸¹⁷ Cazes E, Giron-Michel J, Baouz S, Doucet C, Cagnoni F, Oddera S, Korner M, Dasic G, Testi R, Azzarone B, Canonica G W. Novel anti-inflammatory effects of the inhaled corticosteroid fluticasone propionate during lung myofibroblastic differentiation. J. Immunol. 2001 Nov. 1; 167(9):5329-37.
⁸¹⁸ Jaffuel D, Demoly P, Gougat C, Balaguer P, Mautino G, Godard P, Bousquet I, Mathieu M. Transcriptional potencies of inhaled glucocorticoids. Am J Respir Crit Care Med. 2000 July; 162(1):57-63.
⁸¹⁹ Hart L, Lim S, Adcock I, Barnes P J, Chung K F. Effects of inhaled corticosteroid therapy on expression and DNA-binding activity of nuclear factor kappaB in asthma. Am J Respir Crit Care Med. 2000 January; 161(1):224-31.
⁸²⁰ Bocchino V, Bertorelli G, Zhuo X, Grima P, Di Comite V, Damia R, Chetta A, Del Donno M, Foresi A, Casalini A, Testi R, Olivieri D. Short-term treatment with a low dose of inhaled fluticasone propionate decreases the number of CD1a+dendritic cells in asthmatic airways. Pulm Pharmacol Ther. 1997 October-December; 10(5-6):253-9.
⁸²¹ Lawrence T E, Millecchia L L, Fedan J S. Fluticasone propionate and pentamidine isethionate decrease airway hyperreactivity, pulmonary eosinophilia and pulmonary dendritic cell response in a guinea pig model of asthma. J Pharmacol Exp Ther. 1998 January; 284(1):222-7.
⁸²² Johnson M. Development of fluticasone propionate and comparison with other inhaled corticosteroids. J Allergy Clin Immunol. 1998 April; 101(4 Pt 2):S434-9. Review.
⁸²³ Fokkens W J, Godthelp T, Holm A F, Blom H, Klein-Jan A. Allergic rhinitis and inflammation: the effect of nasal corticosteroid therapy. Allergy 1997; 52(36 Suppl):29-32.
⁸²⁴ Nelson D J, McWilliam A S, Haining S, Holt P G. Modulation of airway intraepithelial dendritic cells following exposure to steroids. Am J Respir Crit Care Med. 1995 February; 151(2 Pt 1):475-81.
⁸²⁵ Moller G M, Overbeek S E, Van Helden-Meeuwsen C G, Van Haarst J M, Prens E P, Mulder P G, Postma D S, Hoogsteden H C. Increased numbers of dendritic cells in the bronchial mucosa of atopic asthmatic patients: downregulation by inhaled corticosteroids. Clin Exp Allergy. 1996 May; 26(5):517-24.
⁸²⁶ Mokdad A H, Serdula M K, Dietz W H, Bowman B A, Marks J S, Koplan J P. The spread of the obesity epidemic in the United States, 1991-1998. JAMA. 1999 Oct. 27; 282(16):1519-22.
⁸²⁷ Weinsier R L, Hunter G R, Heini A F, Goran M I, Sell S M. The etiology of obesity: relative contribution of metabolic factors, diet, and physical activity. Am J. Med. 1998 August; 105(2):145-50.
⁸²⁸ Heini A F, Weinsier R L. Divergent trends in obesity and fat intake patterns: the American paradox. Am J. Med. 1997 March; 102(3):259-64.
⁸²⁹ Hill J O, Peters J C. Environmental contributions to the obesity epidemic. Science. 1998 May 29; 280(5368): 1371-4.
⁸³⁰ Hebebrand J, Wulftange H, Goerg T, Ziegler A, Hinney A, Barth N, Mayer H, Remschmidt H. Epidemic obesity: are genetic factors involved via increased rates of assortative mating? Int J Obes Relat Metab Disord. 2000 March; 24(3):345-53.
⁸³¹ Koplan J P, Dietz W H. Caloric imbalance and public health policy. JAMA. 1999 Oct. 27; 282(16):1579-81.
⁸³² Gabbay D M. What is a logical system. In Gabbay D M, editor, What is a logical system? Clarendon Press, Oxford, 1994. pp 179-216.
⁸³³ Weisz P. Elements of Biology (New York: McGraw-Hill). 1965. p 8
⁸³⁴ Beattie J H, Wood A M, Newman A M, Bremner I, Choo K H A, Michalska A E, Duncan J S, Trayhum P. Obesity and hyperleptinemia in metallothionein (-I and -II) null mice. Proc. Natl. Acad. Sci. USA 1998 95(1): 358-363.
⁸³⁵ Yu M, Yang X Y, Schmidt T, Chinenov Y, Wang R, Martin M E. GA-binding protein-dependent transcription initiator elements. Effect of helical spacing between polyomavirus enhancer a factor 3(PEA3)/Ets-binding sites on initiator activity. J Biol. Chem. 1997 Nov. 14; 272(46):29060-7.
⁸³⁶ Talmud P J, Palmen J, Walker M. Identification of genetic variation in the human hormone-sensitive lipase gene and 5′ sequences: homology of 5′ sequences with mouse promoter and identification of potential regulatory elements. Biochem Biophys Res Commun. 1998 Nov. 27; 252(3):661-8.
⁸³⁷ Grober J, Laurell H, Blaise R, Fabry B, Schaak S, Holm C, Langin D. Characterization of the promoter of human adipocyte hormone-sensitive lipase. Biochem J. 1997 Dec. 1; 328 (Pt 2):453-61.
⁸³⁸ Blaise R, Grober J, Rouet P, Tavernier G, Daegelen D, Langin D. Testis expression of hormone-sensitive lipase is conferred by a specific promoter that contains four regions binding testicular nuclear proteins. J Biol. Chem. 1999 Apr. 2; 274(14):9327-34.
⁸³⁹ Kawamura M, Jensen D F, Wancewicz E V, Joy L L, Khoo J C, Steinberg D. Hormone-sensitive lipase in differentiated 3T3-L1 cells and its activation by cyclic AMP-dependent protein kinase. Proc Natl Acad Sci USA. 1981 February; 78(2):732-6.
⁸⁴⁰ Gordeladze J O, Hovik K E, Merendino J J, Hermouet S, Gutkind S, Accili D. Effect of activating and inactivating mutations of Gs- and Gi2-alpha protein subunits on growth and differentiation of 3T3-L1 preadipocytes. J Cell Biochem. 1997 February; 64(2):242-57.
⁸⁴¹ Osuga J, Ishibashi S, Oka T, Yagyu H, Tozawa R, Fujimoto A, Shionoiri F, Yahagi N, Kraemer F B, Tsutsumi O, Yamada N. Targeted disruption of hormone-sensitive lipase results in male sterility and adipocyte hypertrophy, but not in obesity. Proc Natl Acad Sci USA. 2000 Jan. 18; 97(2):787-92.
⁸⁴² Large V, Reynisdottir S, Langin D, Fredby K, Klannemark M, Holm C, Amer P. Decreased expression and function of adipocyte hormone-sensitive lipase in subcutaneous fat cells of obese subjects. J Lipid Res. 1999 November; 40(11):2059-66.
⁸⁴³ Elizalde M, Ryden M, van Harmelen V, Eneroth P, Gyllenhammar H, Holm C, Ramel S, Olund A, Amer P, Andersson K. Expression of nitric oxide synthases in subcutaneous adipose tissue of nonobese and obese humans. J Lipid Res. 2000 August; 41(8):1244-51.
⁸⁴⁴ Garaulet M, Perez-Llamas F, Zamora S, Tebar F J. Interrelation between serum lipid profile, serum hormones and other components of the metabolic syndrome. J Physiol Biochem. 2002 September; 58(3):151-60.
⁸⁴⁵ Classon M, Kennedy B K, Mulloy R, Harlow E. Opposing roles of pRB and p 107 in adipocyte differentiation. Proc Natl Acad Sci USA. 2000 Sep. 26; 97(20): 10826-31.
⁸⁴⁶ Puigserver P, Ribot J. Serra F, Gianotti M, Bonet M L, Nadal-Ginard B, Palou A. Involvement of the retinoblastoma protein in brown and white adipocyte cell differentiation: functional and physical association with the adipogenic transcription factor C/EBPalpha. Eur J Cell Biol: 1998 October; 77(2):117-23.
⁸⁴⁷ Richon V M, Rilkind R A, Marks P A. Expression and phosphorylation of the retinoblastoma protein during induced differentiation of murine erythroleukemia cells. Cell Growth Differ. 1992 July; 3(7):413-20.
⁸⁴⁸ Roncari D A, Kindler S, Hollenberg C H. Excessive proliferation in culture of reverted adipocytes from massively obese persons. Metabolism. 1986 January; 35(1):1-4.
⁸⁴⁹ Roncari D A, Lau D C, Kindler S. Exaggerated replication in culture of adipocyte precursors from massively obese persons. Metabolism. 1981 May; 30(5):425-7.
⁸⁵⁰ Dhurandhar N V, Israel B A, Kolesar J M, Mayhew G F, Cook M E, Atkinson R L. Increased adiposity in animals due to a human virus. Int J Obes Relat Metab Disord. 2000 August; 24(8):989-96.
⁸⁵¹ Behrens G M, Stoll M, Schmidt R E. Lipodystrophy syndrome in HIV infection: what is it, what causes it and how can it be managed? Drug Saf. 2000 July; 23(1):57-76.
⁸⁵² Engelson E S, Kotler D P, Tan Y, Agin D, Wang J, Pierson R N Jr, Heymsfield S B. Fat distribution in HIV-infected patients reporting truncal enlargement quantified by whole-body magnetic resonance imaging. Am J Clin Nutr. 1999 June; 69(6): 1162-9.
⁸⁵³ Hui D Y. HIV protease inhibitors and atherosclerosis. J Clin Invest. 2003 February; 111(3):317-8.
⁸⁵⁴ Rabinstein A A. Stroke in HIV-Infected Patients: A Clinical Perspective. Cerebrovasc Dis. 2003; 15(1-2):37-44.
⁸⁵⁵ Beregszaszi M, Jaquet D, Levine M, Ortega-Rodriguez E, Baltakse V, Polak M, Levy-Marchal C. Severe insulin resistance contrasting with mild anthropometric changes in the adipose tissue of HIV-infected children with lipohypertrophy. Int J Obes Relat Metab Disord. 2003 January; 27(1):25-30.
⁸⁵⁶ Madamanchi N R, Patterson C, Runge M S. HIV therapies and atherosclerosis: answers or questions? Arterioscler Thromb Vasc Biol. 2002 Nov. 1; 22(11):1758-60. Review.
⁸⁵⁷ Seminari E, Pan A, Voltini G, Carnevale G, Maserati R, Minoli L, Meneghetti G, Tinelli C, Testa S. Assessment of atherosclerosis using carotid ultrasonography in a cohort of HIV-positive patients treated with protease inhibitors. Atherosclerosis. 2002 June; 162(2):433-8.
⁸⁵⁸ Depairon M, Chessex S, Sudre P, Rodondi N, Doser N, Chave J P, Riesen W, Nicod P, Darioli R, Telenti A, Mooser V; Swiss HIV Cohort Study. Premature atherosclerosis in HIV-infected individuals—focus on protease inhibitor therapy. AIDS 2001 Feb. 16; 15(3):329-34.
⁸⁵⁹ Norris S, Kosar Y, Donaldson N, Smith H M, Zolfino T, O'Grady J G, Muiesan P, Rela M, Heaton N. Cytomegalovirus infection after liver transplantation: viral load as a guide to treating clinical infection. Transplantation. 2002 Aug. 27; 74(4):527-31.
⁸⁶⁰ Kogan-Liberman D, Burroughs M, Emre S, Moscona A, Shneider B L. The role of quantitative Epstein-Barr virus polymerase chain reaction and preemptive immunosuppression reduction in pediatric liver transplantation: a preliminary experience. J Pediatr Gastroenterol Nutr. 2001 October; 33(4):445-9.
⁸⁶¹ Rao M, Finny G J, Abraham P, Juneja R, Thomas P P, Jacob C K, Sridharan G. Cytomegalovirus infection in a seroendemic renal transplant population: a longitudinal study of virological markers. Nephron. 2000 April; 84(4):367-73.
⁸⁶² Baum C L, Thielke K, Westin E, Kogan E, Cicalese L, Benedetti E. Predictors of weight gain and cardiovascular risk in a cohort of racially diverse kidney transplant recipients. Nutrition. 2002 February; 18(2):139-46.
⁸⁶³ Richardson R A, Garden O J, Davidson H I. Reduction in energy expenditure after liver transplantation. Nutrition. 2001 July-August; 17(7-8):585-9.
⁸⁶⁴ Clunk J M, Lin C Y, Curtis J J. Variables affecting weight gain in renal transplant recipients. Am J Kidney Dis. 2001 August; 38(2):349-53.
⁸⁶⁵ van den Ham E C, Kooman J P, Christiaans M H, Leunissen K M, van Hooff J P. Posttransplantation weight gain is predominantly due to an increase in body fat mass. Transplantation. 2000 Jul. 15; 70(1):241-2.
⁸⁶⁶ Mor E, Facklam D, Hasse J, Sheiner P, Emre S. Schwartz M, Miller C. Weight gain and lipid profile changes in liver transplant recipients: long-term results of the American FK506 Multicenter Study. Transplant Proc. 1995 February; 27(1):1126.
⁸⁶⁷ Johnson C P, Gallagher-Lepak S, Zhu Y R, Porth C, Kelber S, Roza A M, Adams M B. Factors influencing weight gain after renal transplantation. Transplantation. 1993 October; 56(4):822-7.
⁸⁶⁸ Palmer M, Schaffner F, Thung S N. Excessive weight gain after liver transplantation. Transplantation. 1991 April; 51 (4):797-800.
⁸⁶⁹ Tebourbi L, Emile J F, Cerutti 1. Cyclophosphamide-immunodepressed FVB/N mice: potentiating the effects of testicular cytomegalovirus infection. Intervirology. 2002; 45(2):119-24.
⁸⁷⁰ Palmon A, Tel-or S, Shai E, Rager-Zisman B, Burstein Y. Development of a highly sensitive quantitative competitive PCR assay for the detection of murine cytomegalovirus DNA. J Virol Methods. 2000 May; 86(2): 107-14.
⁸⁷¹ Qamruddin A O, Oppenheim B A, Guiver M, Mutton K J, Chopra R. Screening for cytomegalovirus (CMV) infection in allogeneic bone marrow transplantation using a quantitative whole blood polymerase chain reaction (PCR) method: analysis of potential risk factors for CMV infection. Bone Marrow Transplant 2001 February; 27(3):301-6.
⁸⁷² Schmader K E, Rahija R, Porter K R, Daley G, Hamilton J D. Aging and reactivation of latent murine cytomegalovirus. J Infect Dis 1992 December; 166(6):1403-7.
⁸⁷³ Price P, Hopkins R M, Teo H K, Papadimitriou J M, Shellam G R. Modulation of immunocompetence by cyclosporin A, cyclophosphamide or protein malnutrition potentiates murine cytomegalovirus pneumonitis. Pathol Res Pract. 1991 December; 187(8):993-1000.
⁸⁷⁴ Smee D F, Burger R A, Coombs J, Huffman J H, Sidwell R W. Progressive murine cytomegalovirus disease after termination of ganciclovir therapy in mice immunosuppressed by cyclophosphamide treatment. J Infect Dis. 1991 November; 164(5):958-61.
⁸⁷⁵ Bale J F Jr, O'Neil M E, Folberg R. Murine cytomegalovirus ocular infection in immunocompetent and cyclophosphamide-treated mice. Potentiation of ocular infection by cyclophosphamide. Invest Ophthalmol Vis Sci. 1991 May; 32(6):1749-56.
⁸⁷⁶ Del Rio G, Zironi S, Valeriani L, Menozzi R, Bondi M, Bertolini M, Piccinini L, Banzi M C, Federico M. Weight gain in women with breast cancer treated with adjuvant cyclophosphomide, methotrexate and 5-fluorouracil. Analysis of resting energy expenditure and body composition. Breast Cancer Res Treat. 2002 June; 73(3):267-73.
⁸⁷⁷ Lankester K J, Phillips J E, Lawton P A. Weight gain during adjuvant and neoadjuvant chemotherapy for breast cancer: an audit of 100 women receiving FEC or CMF chemotherapy. Clin Oncol (R Coll Radiol). 2002 February; 14(1):64-7.
⁸⁷⁸ Aslani A, Smith R C, Allen B J, Pavlakis N, Levi J A. Changes in body composition during breast cancer chemotherapy with the CMF-regimen. Breast Cancer Res Treat. 1999 October; 57(3):285-90.
⁸⁷⁹ Sitzia J, Huggins L. Side effects of cyclophosphamide, methotrexate, and 5-fluorouracil (CMF) chemotherapy for breast cancer. Cancer Pract. 1998 January-February; 6(1):13-21.
⁸⁸⁰ Demark-Wahnefried W, Hars V, Conaway M R, Havlin K, Rimer B K, McElveen G, Winer E P. Reduced rates of metabolism and decreased physical activity in breast cancer patients receiving adjuvant chemotherapy. Am J Clin Nutr 1997 May; 65(5):1495-501.
⁸⁸¹ Kutynec C L, McCargar L, Barr S I, Hislop T G. Energy balance in women with breast cancer during adjuvant treatment. J Am Diet Assoc. 1999 October; 99(10):1222-7.
⁸⁸² Dubbert P M, Carithers T, Sumner A E, Barbour K A, Clark B L, Hall J E, Crook E D. Obesity, physical inactivity, and risk for cardiovascular disease. Am J Med Sci. 2002 September; 324(3): 116-26. Review.
⁸⁸³ Wilson P W, Kannel W B. Obesity, diabetes, and risk of cardiovascular disease in the elderly. Am J Geriatr Cardiol. 2002 March-April; 11(2):119-23,125. Review.
⁸⁸⁴ Jousilahti P, Tuomilehto J, Vartiainen E, Pekkanen J, Puska P. Body weight, cardiovascular risk factors, and coronary mortality. 15-year follow-up of middle-aged men and women in eastern Finland. Circulation. 1996 Apr. 1; 93(7): 1372-9.
⁸⁸⁵ Licata G, Scaglione R, Avellone G, Parrinello G, Merlino G, Corrao S. Obesity, hypertension and atherosclerosis. Int Angiol 1993 December; 12(4):326-30.
⁸⁸⁶ Kannel W B, Cupples L A, Ramaswami R, Stokes J 3rd, Kreger B E, Higgins M. Regional obesity and risk of cardiovascular disease; the Framingham Study. J Clin Epidemiol. 1991; 44(2):183-90.
⁸⁸⁷ Hubert H B, Feinleib M, McNamara P M, Castelli W P. Obesity as an independent risk factor for cardiovascular disease: a 26-year follow-up of participants in the Framingham Heart Study. Circulation. 1983 May; 67(5):968-77.
⁸⁸⁸ Gordon T, Kannel W B. Obesity and cardiovascular diseases: the Framingham study. Clin Endocrinol Metab. 1976 July; 5(2):367-75.
⁸⁸⁹ Kannel W B, LeBauer E J, Dawber T R, McNamara P M. Relation of body weight to development of coronary heart disease. The Framingham study. Circulation. 1967 April; 35(4):734-44.
⁸⁹⁰ Bianchini F, Kaaks R, Vainio H. Overweight, obesity, and cancer risk. Lancet Oncol. 2002 September; 3(9):565-74. Review.
⁸⁹¹ Bergstrom A, Pisani P, Tenet V, Wolk A, Adami H O. Overweight as an avoidable cause of cancer in Europe. Int J Cancer. 2001 Feb. 1; 91(3):421-30.
⁸⁹² McTiernan A. Associations between energy balance and body mass index and risk of breast carcinoma in women from diverse racial and ethnic backgrounds in the U.S. Cancer. 2000 Mar. 1; 88(5 Suppl):1248-55. Review.
⁸⁹³ Guthrie N, Carroll K K. Specific versus non-specific effects of dietary fat on carcinogenesis. Prog Lipid Res. 1999 May; 38(3):261-71.
⁸⁹⁴ Carroll K K. Obesity as a risk factor for certain types of cancer. Lipids. 1998 November; 33(11):1055-9.
⁸⁹⁵ Kasahara S, Wago H, Cooper E L. Dissociation of innate and adaptive immunity by UVB irradiation. Int J Immunopathol Pharmacol. 2002 January-April; 15(1): 1-11.
⁸⁹⁶ Kasahara S, Aizawa K, Okamiya M, Kazuno N, Mutoh S, Fugo H, Cooper E L, Wago H. UVB irradiation suppresses cytokine production and innate cellular immune functions in mice. Cytokine. 2001 Apr. 21; 14(2):104-11.
⁸⁹⁷ Garssen J, Vandebriel R J, De Gruijl F R, Wolvers D A, Van Dijk M, Fluitman A, Van Loveren H. UVB exposure-induced systemic modulation of Th1- and Th2-mediated immune responses. Immunology 1999 July; 97(3):506-14.
⁸⁹⁸ Keadle T L, Morrison L A, Morris J L, Pepose J S, Stuart P M. Therapeutic immunization with a virion host shutoff-defective, replication-incompetent herpes simplex virus type I strain limits recurrent herpetic ocular infection. J. Virol. 2002 April; 76(8):3615-25.
⁸⁹⁹ El-Ghorr A A, Norval M. The effect of UV-B irradiation on primary and secondary HSV-1 infections in interleukin-4 knockout mice. Arch Dermatol Res. 1999 July-August; 291(7-8):459-65.
⁹⁰⁰ Walker J, Laycock K A, Pepose J S, Leib D A. Postexposure vaccination with a virion host shutoff defective mutant decreases UV-B radiation-induced ocular herpes simplex virus shedding in mice. Vaccine 1998 January; 16(1):6-8.
⁹⁰¹ Blatt A N, Laycock K A, Brady R H, Traynor P, Krogstad D J, Pepose J S. Prophylactic acyclovir effectively decreases herpes simplex virus type I reactivation after exposure of latently infected mice to ultraviolet B. Invest Ophthalmol Vis Sci. 1993 November; 34(12):3459-65.
⁹⁰² Miller J K, Laycock K A, Nash M M, Pepose J S. Corneal Langerhans cell dynamics after herpes simplex virus reactivation. Invest Ophthalmol Vis Sci. 1993 June; 34(7):2282-90.
⁹⁰³ Rooney J F, Straus S E, Mannix M L, Wohlenberg C R, Banks S, Jagannath S, Brauer J E, Notkins A L. UV light-induced reactivation of herpes simplex virus type 2 and prevention by acyclovir. J Infect Dis. 1992 September; 166(3):500-6.
⁹⁰⁴ Laycock K A, Lee S F, Brady R H, Pepose J S. Characterization of a murine model of recurrent herpes simplex viral keratitis induced by ultraviolet B radiation. Invest Ophthalmol Vis Sci. 1991 September; 32(10):2741-6.
⁹⁰⁵ Clydesdale G J, Dandie G W, Muller H K. Ultraviolet light induced injury: immunological and inflammatory effects. Immunol Cell Biol. 2001 December; 79(6):547-68. Review.
⁹⁰⁶ Garssen J, van Loveren H. Effects of ultraviolet exposure on the immune system. Crit Rev Immunol. 2001; 21(4):359-97. Review.
⁹⁰⁷ Kaufman F R. Type 2 diabetes mellitus in children and youth: a new epidemic. J Pediatr Endocrinol Metab. 2002 May; 15 Suppl 2:737-44. Review.
⁹⁰⁸ Seidell J C. Obesity, insulin resistance and diabetes—a worldwide epidemic. Br I Nutr. 2000 March; 83 Suppl 1:S5-8. Review.
⁹⁰⁹ Rosenbloom A L, Joe J R, Young R S, Winter W E. Emerging epidemic of type 2 diabetes in youth. Diabetes Care. 1999 February; 22(2):345-54. Review.
⁹¹⁰ Jovanovic L, Gondos B. Type 2 diabetes: the epidemic of the new millennium. Ann Clin Lab Sci. 1999 January-March; 29(1):33-42. Review.
⁹¹¹ Deedwania P C. Diabetes and vascular disease: common links in the emerging epidemic of coronary artery disease. Am J Cardiol. 2003 Jan. 1; 91(1):68-71. Review.
⁹¹² Bonow R O, Smaha L A, Smith S C Jr, Mensah G A, Lenfant C. World Heart Day 2002: the international burden of cardiovascular disease: responding to the emerging global epidemic. Circulation 2002 Sep. 24; 106(13):1602-5.
⁹¹³ Reddy K S, Yusuf S. Emerging epidemic of cardiovascular disease in developing countries. Circulation. 1998 Feb. 17; 97(6):596-601. Review.
⁹¹⁴ el-Serag H B. The epidemic of esophageal adenocarcinoma. Gastroenterol Clin North Am. 2002 June; 31(2):421-40, viii. Review.
⁹¹⁵ Limmer B L. Nonmelanoma skin cancer: today's epidemic. Tex Med. 2001 February; 97(2):56-8. Review.
⁹¹⁶ Dennis L K. Analysis of the melanoma epidemic, both apparent and real: data from the 1973 through 1994 surveillance, epidemiology, and end results program registry. Arch Dermatol. 1999 March; 135(3):275-80.
⁹¹⁷ Weisenburger D D. Epidemiology of non-Hodgkin's lymphoma: recent findings regarding an emerging epidemic. Ann Oncol. 1994; 5 Suppl 1: 19-24. Review.
⁹¹⁸ Kheradmand F, Rishi K, Corry D B. Environmental contributions to the allergic asthma epidemic. Environ Health Perspect. 2002 August; 110 Suppl 4:553-6. Review.
⁹¹⁹ Umetsu D T, Mclntire J J, Akbari O, Macaubas C, DeKruyff R H. Asthma: an epidemic of dysregulated immunity. Nat Immunol. 2002 August; 3(8):715-20. Review.
⁹²⁰ Holgate S T. The epidemic of allergy and asthma. Nature. 1999 Nov. 25; 402(6760 Suppl):B2-4.
⁹²¹ Silink M. Childhood diabetes: a global perspective. Horm Res 2002; 57 Suppl 1:1-5.
⁹²² Kida K, Mimura G, Ito T, Murakami K, Ashkenazi I, Laron Z. Incidence of Type I diabetes mellitus in children aged 0-14 in Japan, 1986-1990, including an analysis for seasonality of onset and month of birth: JDS study. The Data Committee for Childhood Diabetes of the Japan Diabetes Society (JDS). Diabet Med 2000 January; 17(1):59-63.
⁹²³ Das U N. Is metabolic syndrome X an inflammatory condition? Exp Biol. Med (Maywood). 2002 December; 227(11):989-97. Review.
⁹²⁴ Das U N. Metabolic syndrome X is common in South Asians, but why and how? Nutrition. 2002 September; 18(9):774-6.
⁹²⁵ Das U N. Is obesity an inflammatory condition? Nutrition. 2001 November-December; 17(11-12):953-66. Review.
⁹²⁶ Yamashita T, Murakami T, Otani S, Kuwajima M, Shima K. Leptin receptor signal transduction: OBRa and OBRb of fa type. Biochem Biophys Res Commun. 1998 May 29; 246(3):752-9.
⁹²⁷ Clement K, Vaisse C, Lahlou N, Cabrol S, Pelloux V, Cassuto D, Gourmelen M, Dina C, Chambaz J, Lacorte J M, Basdevant A, Bougneres P, Lebouc Y, Froguel P, Guy-Grand B. A mutation in the human leptin receptor gene causes obesity and pituitary dysfunction. Nature. 1998 Mar. 26; 392(6674):398-401.
⁹²⁸ Chen H, Charlat O, Tartaglia L A, Woolf E A, Weng X, Ellis S J, Lakey N D, Culpepper J, Moore K J, Breitbart R E, Duyk G M, Tepper R I, Morgenstern J P. Evidence that the diabetes gene encodes the leptin receptor: identification of a mutation in the leptin receptor gene in db/db mice. Cell 1996 Feb. 9; 84(3):491-5.
⁹²⁹ da Silva B A, Bjorbaek C, Uotani S, Flier J S. Functional properties of leptin receptor isoforms containing the gln-->pro extracellular domain mutation of the fatty rat. Endocrinology. 1998 September; 139(9):3681-90.
⁹³⁰ Caruso A, Fortini A, Fulghesu A M, Pistilli E, Cucinelli F, Lanzone A, Mancuso S. Ovarian sensitivity to follicle-stimulating hormone during the follicular phase of the human menstrual cycle and in patients with polycystic ovarian syndrome. Fertil Steril. 1993 January; 59(1):115-20.
⁹³¹ Wronski T J, Schenck P A, Cintron M, Walsh C C. Effect of body weight on osteopenia in ovariectomized rats. Calcif Tissue Int. 1987 March; 40(3):155-9.
⁹³² Flaherty S F, Golenbock D T, Milham F H, Ingalls R R. CD11/CD18 leukocyte integrins: new signaling receptors for bacterial endotoxin. J Surg Res. 1997 November; 73(1):85-9.
⁹³³ Ingalls R R, Golenbock D T. CD11c/CD18, a transmembrane signaling receptor for lipopolysaccharide. J Exp Med. 1995 Apr. 1; 181(4): 1473-9.
⁹³⁴ Ingalls R R, Arnaout M A, Golenbock D T. Outside-in signaling by lipopolysaccharide through a tailless integrin. J. Immunol. 1997 Jul. 1; 159(1):433-8.
⁹³⁵ Dong Z M, Gutierrez-Ramos J C, Coxon A, Mayadas T N, Wagner D D. A new class of obesity genes encodes leukocyte adhesion receptors. Proc Natl Acad Sci USA. 1997 Jul. 8; 94(14):7526-30.
⁹³⁶ Ledikwe J H, Smiciklas-Wright H. Mitchell D C, Jensen G L, Friedmann J M, Still C D. Nutritional risk assessment and obesity in rural older adults: a sex difference. Am J Clin Nutr. 2003 March; 77(3):551-8.
⁹³⁷ Ozata M, Mergen M, Oktenli C, Aydin A, Yavuz Sanisoglu S. Bolu E, Yilmaz M I, Sayal A, Isimer A, Ozdemir I C. Increased oxidative stress and hypozincemia in male obesity. Clin Biochem. 2002 November; 35(8):627-31.
⁹³⁸ Marreiro Ddo N, Fisberg M, Cozzolino S M. Zinc nutritional status in obese children and adolescents. Biol Trace Elem Res. 2002 May; 86(2):107-22.
⁹³⁹ Singh R B, Niaz M A, Rastogi S S, Bajaj S, Gaoli Z, Shoumin Z. Current zinc intake and risk of diabetes and coronary artery disease and factors associated with insulin resistance in rural and urban populations of North India. J Am Coll Nutr. 1998 December; 17(6):564-70.
⁹⁴⁰ Ilyin S E, Plata-Salaman C R. Molecular regulation of the brain interleukin-1 beta system in obese (fa/fa) and lean (Fa/Fa) Zucker rats. Brain Res Mol Brain Res. 1996 Dec. 31; 43(1-2):209-18.
⁹⁴¹ del Rey A, Besedovsky H. Antidiabetic effects of interleukin 1. Proc Natl Acad Sci USA 1989 August; 86(15):5943-7.
⁹⁴² Luheshi G N, Gardner J D, Rushforth D A, Loudon A S, Rothwell N J. Leptin actions on food intake and body temperature are mediated by IL-1. Proc Natl Acad Sci USA 1999 Jun. 8; 96(12):7047-52.
⁹⁴³ Plata-Salaman C R, Vasselli J R, Sonti G. Differential responsiveness of obese (fa/fa) and lean (Fa/Fa) Zucker rats to cytokine-induced anorexia. Obes Res 1997 January; 5(1):36-42.
⁹⁴⁴ Faggioni R, Fuller J, Moser A, Feingold K R, Grunfeld C. LPS-induced anorexia in leptin-deficient (ob/ob) and leptin receptor-deficient (db/db) mice. Am J. Physiol. 1997 July; 273(1 Pt 2):R181-6.
⁹⁴⁵ Hipskind R A, Bilbe G. MAP kinase signaling cascades and gene expression in osteoblasts. Front Biosci. 1998 Aug. 1; 3:D804-16.
⁹⁴⁶ Espinos E, Le Van Thai A, Pomies C, Weber M J. Cooperation between phosphorylation and acetylation processes in transcriptional control. Mol Cell Biol 1999 May; 19(5):3474-84.
⁹⁴⁷ Shiraishi M, Hirasawa N, Kobayashi Y, Oikawa S, Murakami A, Ohuchi K. Participation of mitogen-activated protein kinase in thapsigargin- and TPA-induced histamine production in murine macrophage RAW 264.7 cells. Br J Pharmacol 2000 February; 129(3):515-24.
⁹⁴⁸ Herrera R, Hubbell S, Decker S, Petruzzelli L. A role for the MEK/MAPK pathway in PMA-induced cell cycle arrest: modulation of megakaryocytic differentiation of K562 cells. Exp Cell Res 1998 Feb. 1; 238(2):407-14.
⁹⁴⁹ Stadheim T A, Kucera G L. Extracellular signal-regulated kinase (ERK) activity is required for TPA-mediated inhibition of drug-induced apoptosis. Biochem Biophys Res Commun 1998 Apr. 7; 245(1):266-71.
⁹⁵⁰ Yen A, Roberson M S, Varvayanis S. Retinoic acid selectively activates the ERK2 but not JNK/SAPK or p38 MAP kinases when inducing myeloid differentiation. In Vitro Cell Dev Biol Anim. 1999 October; 35(9):527-32.
⁹⁵¹ Liu M K, Brownsey R W, Reiner N E. Γ interferon induces rapid and coordinate activation of mitogen-activated protein kinase (extracellular signal-regulated kinase) and calcium-independent protein kinase C in human monocytes. Infect Immun, July 1994, 2722-2731, Vol 62, No. 7.
⁹⁵² Nishiya T, Uehara T, Edamatsu H, Kaziro Y, Itoh H, Nomura Y. Activation of Stat1 and subsequent transcription of inducible nitric oxide synthase gene in C6 glioma cells is independent of interferon-γ-induced MAPK activation that is mediated by p21ras. FEBS Lett 1997 May 12; 408(1):33-8.
⁹⁵³ Lessor T, Yoo J Y, Davis M, Hamburger A W. Regulation of heregulin beta1-induced differentiation in a human breast carcinoma cell line by the extracellular-regulated kinase (ERK) pathway. J Cell Biochem 1998 Sep. 15; 70(4):587-95.
⁹⁵⁴ Marte B M, Graus-Porta D, Jeschke M, Fabbro D, Hynes N E, Taverna D. NDF/heregulin activates MAP kinase and p70/p85 S6 kinase during proliferation or differentiation of mammary epithelial cells. Oncogene 1995 Jan. 5; 10(1):161-75.
⁹⁵⁵ Sepp-Lorenzino L, Eberhard I, Ma Z, Cho C, Serve H, Liu F, Rosen N, Lupu R. Signal transduction pathways induced by heregulin in MDA-MB-453 breast cancer cells. Oncogene 1996 Apr. 18; 12(8):1679-87.
⁹⁵⁶ Fiddes R J, Janes P W, Sivertsen S P, Sutherland R L, Musgrove E A, Daly R J. Inhibition of the MAP kinase cascade blocks heregulin-induced cell cycle progression in T-47D human breast cancer cells. Oncogene 1998 May 28; 16(21):2803-13.
⁹⁵⁷ Park J A, Koh J Y. Induction of an immediate early gene egr-1 by zinc through extracellular signal-regulated kinase activation in cortical culture: its role in zinc-induced neuronal death. J. Neurochem. 1999 August; 73(2):450-6.
⁹⁵⁸ Kiss Z, Crilly K S, Tomono M. Bombesin and zinc enhance the synergistic mitogenic effects of insulin and phosphocholine by a MAP kinase-dependent mechanism in Swiss 3T3 cells. FEBS Lett. 1997 Sep. 22; 415(1):71-4.
⁹⁵⁹ Wu W, Graves L M, Jaspers I, Devlin R B, Reed W, Samet J M. Activation of the EGF receptor signaling pathway in human airway epithelial cells exposed to metals. Am J. Physiol. 1999 November; 277(5 Pt 1):L924-31.
⁹⁶⁰ Samet J M, Graves L M, Quay J, Dailey L A, Devlin R B, Ghio A J, Wu W, Bromberg P A, Reed W. Activation of MAPKs in human bronchial epithelial cells exposed to metals. Am J. Physiol. 1998 September; 275(3 Pt 1):L551-8.
⁹⁶¹ Migliaccio A, Di Domenico M, Castoria G, de Falco A, Bontempo P, Nola E, Auricchio F. Tyrosine kinase/p21ras/MAP-kinase pathway activation by estradiol-receptor complex in MCF-7 cells. EMBO J. 1996 Mar. 15; 15(6):1292-300.
⁹⁶² Ruzycky A L. Effects of 17 beta-estradiol and progesterone on mitogen-activated protein kinase expression and activity in rat uterine smooth muscle. Eur J. Pharmacol. 1996 Apr 11; 300(3):247-54.
⁹⁶³ Nuedling S, Kahlert S, Loebbert K, Meyer R, Vetter H, Grohe C. Differential effects of 17beta-estradiol on mitogen-activated protein kinase pathways in rat cardiomyocytes. FEBS Lett. 1999 Jul. 9; 454(3):271-6.
⁹⁶⁴ Laporte J D, Moore P E, Abraham J H, Maksym G N, Fabry B, Panettieri R A Jr, Shore S A. Role of ERK MAP kinases in responses of cultured human airway smooth muscle cells to IL-1beta. Am J. Physiol. 1999 November; 277(5 Pt 1):L943-51.
⁹⁶⁵ Larsen C M, Wadt K A, Juhl L F, Andersen H U, Karlsen A E, Su M S, Seedorf K, Shapiro L, Dinarello C A, Mandrup-Poulsen T. Interleukin-1beta-induced rat pancreatic islet nitric oxide synthesis requires both the p38 and extracellular signal-regulated kinase 1/2 mitogen-activated protein kinases. J Biol. Chem. 1998 Jun. 12; 273(24):15294-300.
⁹⁶⁶ Daeipour M, Kumar G, Amaral M C, Nel A E. Recombinant IL-6 activates p42 and p44 mitogen-activated protein kinases in the IL-6 responsive B cell line, AF-10. J. Immunol. 1993 Jun. 1; 150(11):4743-53.
⁹⁶⁷ Leonard M, Ryan M P, Watson A J, Schramek H, Healy E. Role of MAP kinase pathways in mediating IL-6 production in human primary mesangial and proximal tubular cells. Kidney Int. 1999 October; 56(4):1366-77.
⁹⁶⁸ Hartsough M T, Mulder K M. Transforming growth factor beta activation of p44mapk in proliferating cultures of epithelial cells. J Biol. Chem. 1995 Mar. 31; 270(13):7117-24.
⁹⁶⁹ Yonekura A, Osaki M, Hirota Y, Tsukazaki T, Miyazaki Y, Matsumoto T, Ohtsuru A, Namba H, Shindo H, Yamashita S. Transforming growth factor-beta stimulates articular chondrocyte cell growth through p44/42 MAP kinase (ERK) activation. Endocr J. 1999 August; 46(4):545-53.
⁹⁷⁰ Strakova Z, Copland J A, Lolait S J, Soloff M S. ERK2 mediates oxytocin-stimulated PGE2 synthesis. Am J. Physiol. 1998 April; 274(4 Pt 1):E634-41.
⁹⁷¹ Copland J A, Jeng Y J, Strakova Z, Ives K L, Hellmich M R, Soloff M S. Demonstration of functional oxytocin receptors in human breast Hs578 T cells and their up-regulation through a protein kinase C-dependent pathway. Endocrinology. 1999 May; 140(5):2258-67.
⁹⁷² Hoffmeyer A, Avots A, Flory E, Weber C K, Serfling E, Rapp U R. The GABP-responsive element of the interleukin-2 enhancer is regulated by JNK/SAPK-activating pathways in T lymphocytes. J Biol. Chem. 1998 Apr. 24; 273(17): 10112-9.
⁹⁷³ Tomaras G D, Foster D A, Burrer C M, Taffet S M. ETS transcription factors regulate an enhancer activity in the third intron of TNF-alpha. J Leukoc Biol 1999 July; 66(1):183-93.
⁹⁷⁴ Zhong H, Voll R E, Ghosh S. Phosphorylation of NF-kappa B p65 by PKA stimulates transcriptional activity by promoting a novel bivalent interaction with the coactivator CBP/p300. Mol Cell. 1998 April; 1(5):661-71.
⁹⁷⁵ Bevilacqua M A, Faniello M C, Cimino F, Costanzo F. Okadaic acid stimulates H ferritin transcription in HeLa cells by increasing the interaction between the p300 CO-activator molecule and the transcription factor Bbf. Biochem Biophys Res Commun. 1997 Nov. 7; 240(1):179-82.
⁹⁷⁶ Kuo M H, Allis C D. Roles of histone acetyltransferases and deacetylases in gene regulation. BioEassays 1998 20:615-626.
⁹⁷⁷ Hebbes T R, Clayton A L, Thome A W, Crane-Robinson C. Core histone hyperacetylation co-maps with generalized DNase I sensitivity in the chicken beta-globin chromosomal domain. EMBO J. 1994 Apr. 15; 13(8):1823-30.
⁹⁷⁸ Kuhnert P, Peterhans E, Pauli U. Chromatin structure and DNase I hypersensitivity in the transcriptionally active and inactive porcine tumor necrosis factor gene locus. Nucleic Acids Res. 1992 Apr. 25; 20(8):1943-8.
⁹⁷⁹ Herschlag D, Johnson F B. Synergism in transcriptional activation: a kinetic view. Genes Dev. 1993 February; 7(2):173-9. Review.
⁹⁸⁰ Eckner R, Ewen M E, Newsome D, Gerdes M, DeCaprio J A, Lawrence J B, Livingston D M. Molecular cloning and functional analysis of the adenovirus E1A-associated 300-kD protein (p300) reveals a protein with properties of a transcriptional adaptor. Genes Dev. 1994 Apr. 15; 8(8):869-84.
⁹⁸¹ Martin M E, Chinenov Y, Yu M, Schmidt T K, Yang X Y. Redox regulation of GA-binding protein-alpha DNA binding activity. J Biol. Chem. 1996 Oct 11; 271(41):25617-23.
⁹⁸² Schaeffer L, Duclert N, Huchet-Dymanus M, Changeux J P. Implication of a multisubunit Ets-related transcription factor in synaptic expression of the nicotinic acetylcholine receptor. EMBO J. 1998 Jun. 1; 17(l 1):3078-90.
⁹⁸³ Duclert A, Savatier N, Schaeffer L, Changeux J P. Identification of an element crucial for the sub-synaptic expression of the acetylcholine receptor epsilon-subunit gene. J Biol. Chem. 1996 Jul. 19; 271(29):17433-8.
⁹⁸⁴ Koike S, Schaeffer L, Changeux J P. Identification of a DNA element determining synaptic expression of the mouse acetylcholine receptor delta-subunit gene. Proc Natl Acad Sci USA. 1995 Nov. 7; 92(23):10624-8.
⁹⁸⁵ Fromm L, Burden S J. Synapse-specific and neuregulin-induced transcription require an ets site that binds GABPalpha/GABPbeta. Genes Dev. 1998 Oct. 1; 12(19):3074-83.
⁹⁸⁶ Tansey M G, Chu G C, Merlie J P. ARIA/HRG regulates AChR epsilon subunit gene expression at the neuromuscular synapse via activation of phosphatidylinositol 3-kinase and Ras/MAPK pathway. J. Cell Biol. 1996 July; 134(2):465-76.
⁹⁸⁷ Gramolini A O, Angus L M, Schaeffer L, Burton E A, Tinsley J M, Davies K E, Changeux J P, Jasmin B J. Induction of utrophin gene expression by heregulin in skeletal muscle cells: role of the N-box motif and GA binding protein. Proc Natl Acad Sci USA. 1999 Mar. 16; 96(6):3223-7.
⁹⁸⁸ Maudsley S, Pierce K L, Zamah A M, Miller W E, Ahn S, Daaka Y, Lefkowitz R J, Luttrell L M. The beta(2)-adrenergic receptor mediates extracellular signal-regulated kinase activation via assembly of a multi-receptor complex with the epidermal growth factor receptor. J Biol. Chem. 2000 Mar. 31; 275(13):9572-80.
⁹⁸⁹ Pierce K L, Maudsley S, Daaka Y, Luttrell L M, Lefkowitz R J. Role of endocytosis in the activation of the extracellular signal-regulated kinase cascade by sequestering and nonsequestering G protein-coupled receptors. Proc Natl Acad Sci USA. 2000 Feb. 15; 97(4):1489-94.
⁹⁹⁰ Elorza A, Sarnago S, Mayor F. Jr. Agonist-dependent modulation of G protein-coupled receptor kinase 2 by mitogen-activated protein kinases. Mol Pharmacol. 2000 April; 57(4):778-83.
⁹⁹¹ Luttrell L M, Ferguson S S, Daaka Y, Miller W E, Maudsley S, Della Rocca G J, Lin F, Kawakatsu H, Owada K, Luttrell D K, Caron M G, Lefkowitz R J. Beta-arrestin-dependent formation of beta2 adrenergic receptor-Src protein kinase complexes. Science. 1999 Jan. 29; 283(5402):655-61.
⁹⁹² Daaka Y, Luttrell L M, Ahn S, Della Rocca G J, Ferguson S S, Caron M G, Lefkowitz R J. Essential role for G protein-coupled receptor endocytosis in the activation of mitogen-activated protein kinase. J Biol. Chem. 1998 Jan. 9; 273(2):685-8.
⁹⁹³ Cao W, Luttrell L M, Medvedev A V, Pierce K L, Daniel K W, Dixon T M, Lefkowitz R J, Collins S. Direct Binding of Activated c-Src to the Beta3-Adrenergic Receptor is Required for MAP Kinase Activation. J Biol. Chem. 2000 Sep. 29.
⁹⁹⁴ Gerhardt C C, Gros J, Strosberg A D, Issad T. Stimulation of the extracellular signal-regulated kinase 1/2 pathway by human beta-3 adrenergic receptor: new pharmacological profile and mechanism of activation. Mol Pharmacol. 1999 February; 55(2):255-62.
⁹⁹⁵ Soeder K J, Snedden S K, Cao W, Della Rocca G J, Daniel K W, Luttrell L M, Collins S. The beta3-adrenergic receptor activates mitogen-activated protein kinase in adipocytes through a Gi-dependent mechanism. J Biol. Chem. 1999 Apr. 23; 274(17):12017-22.
⁹⁹⁶ Hellstrom L, Langin D, Reynisdottir S, Dauzats M, Arner P. Adipocyte lipolysis in normal weight subjects with obesity among first-degree relatives. Diabetologia. 1996 August; 39(8):921-8.
⁹⁹⁷ Shimizu Y, Tanishita T, Minokoshi Y, Shimazu T. Activation of mitogen-activated protein kinase by norepinephrine in brown adipocytes from rats. Endocrinology. 1997 January; 138(1):248-53.
⁹⁹⁸ Yarwood S J, Kilgour E, Anderson N G. Cyclic AMP stimulates the phosphorylation and activation of p42 and p44 mitogen-activated protein kinases in 3T3-F442A preadipocytes. Biochem Biophys Res Commun. 1996 Jul. 25; 224(3):734-9.
⁹⁹⁹ Hellstrom L, Reynisdottir S. Influence of heredity for obesity on adipocyte lipolysis in lean and obese subjects. Int J Obes Relat Metab Disord. 2000 March; 24(3):340-4.
¹⁰⁰⁰ Bougneres P, Stunff C L, Pecqueur C, Pinglier E, Adnot P, Ricquier D. In vivo resistance of lipolysis to epinephrine. A new feature of childhood onset obesity. J Clin Invest. 1997 Jun. 1; 99(11):2568-73.
¹⁰⁰¹ Horowitz J F, Klein S. Whole body and abdominal lipolytic sensitivity to epinephrine is suppressed in upper body obese women. Am J Physiol Endocrinol Metab. 2000 June; 278(6):E1144-52.
¹⁰⁰² Kimura T, Takemura M, Nomura S, Nobunaga T, Kubota Y, Inoue T, Hashimoto K, Kumazawa I, Ito Y. Ohashi K, Koyama M, Azuma C, Kitamura Y, Saji F. Expression of oxytocin receptor in human pregnant myometrium. Endocrinology. 1996 February; 137(2):780-5.
¹⁰⁰³ Myerson D, Hackman R C, Nelson J A, Ward D C, McDougall J K. Widespread presence of histologically occult cytomegalovirus. Hum Pathol 1984 May; 15(5):430-9.
¹⁰⁰⁴ Voog E, Ricksten A, Olofsson S, Ternesten A, Ryd W, Kjellstrom C, Forslund O, Lowhagen G B. Demonstration of Epstein-Barr virus DNA and human papillomavirus DNA in acetowhite lesions of the penile skin and the oral mucosa. Int J STD AIDS. 1997 December; 8(12):772-5.
¹⁰⁰⁵ Taylor Y, Melvin W T, Sewell H F, Flannelly G, Walker F. Prevalence of Epstein-Barr virus in the cervix. J Clin Pathol. 1994 January; 47(1):92-3.
¹⁰⁰⁶ Johnson S R, Kolberg B H, Varner M W, Railsback L D. Maternal obesity and pregnancy. Surg Gynecol Obstet. 1987 May; 164(5):431-7.
¹⁰⁰⁷ Chapman D J, Perez-Escamilla R. Identification of risk factors for delayed onset of lactation. J Am Diet Assoc. 1999 April; 99(4):450-4; quiz 455-6.
¹⁰⁰⁸ Donath S M, Amir L H. Does maternal obesity adversely affect breastfeeding initiation and duration? Breastfeed Rev. 2000 November; 8(3):29-33.
¹⁰⁰⁹ Hilson J A, Rasmussen K M, Kjolhede C L. Maternal obesity and breast-feeding success in a rural population of white women. Am J Clin Nutr. 1997 December; 66(6):1371-8.
¹⁰¹⁰ Kahn B B, Flier J S. Obesity and insulin resistance. J Clin Invest. 2000 August; 106(4):473-81.
¹⁰¹¹ Dube M P. Disorders of glucose metabolism in patients infected with human immunodeficiency virus. Clin Infect Dis 2000 December; 31(6):1467-75.
¹⁰¹² Stock S, Granstrom L, Backman L, Matthiesen A S, Uvnas-Moberg K. Elevated plasma levels of oxytocin in obese subjects before and after gastric banding. Int J Obes. 1989; 13(2):213-22.
¹⁰¹³ Yakinci C, Pac A, Kucukbay F Z, Tayfun M, Gul A. Serum zinc, copper, and magnesium levels in obese children. Acta Paediatr Jpn. 1997 June; 39(3):339-41.
¹⁰¹⁴ D'Ocon C, Alonso de Armino V, Frasquet I. Levels of Zn and Cu in the serum of a diabetic population. Rev Esp Fisiol. 1987 September; 43(3):335-8. Spanish.
¹⁰¹⁵ Taneja S K, Mahajan M, Arya P. Excess bioavailability of zinc may cause obesity in humans. Experientia. 1996 Jan. 16; 52(1):31-3.
¹⁰¹⁶ Cauley J A, Gutai J P, Kuller L H, Scott J, Nevitt M C. Black-white differences in serum sex hormones and bone mineral density. Am J Epidemiol. 1994 May 15; 139(10):1035-46.
¹⁰¹⁷ Cauley J A, Gutai J P, Kuller L H, LeDonne D, Powell J G. The epidemiology of serum sex hormones in postmenopausal women. Am J Epidemiol. 1989 June; 129(6):1120-31.
¹⁰¹⁸ de Waard F, Poortman J, de Pedro-Alvarez Ferrero M, Baanders-van Halewijn E A. Weight reduction and oestrogen excretion in obese post-menopausal women. Maturitas. 1982 August; 4(2):155-62.
¹⁰¹⁹ Pickup J C, Crook M A. Is type II diabetes mellitus a disease of the innate immune system? Diabetologia. 1998 October; 41(10):1241-8.
¹⁰²⁰ Pickup J C, Mattock M B, Chusney G D, Burt D. NIDDM as a disease of the innate immune system: association of acute-phase reactants and interleukin-6 with metabolic syndrome X. Diabetologia. 1997 November; 40(11):1286-92.
¹⁰²¹ Allebach E S, Boettiger D, Pacifici M, Adams S L. Control of types 1 and 11 collagen and fibronectin gene expression in chondrocytes delineated by viral transformation. Mol Cell Biol. 1985 May; 5(5):1002-8.
¹⁰²² Parker I M, Smith A A, Gevers W. Absence of α2(1) Procollagen Synthesis in a Clone of SV40-transformed WI-38 Human Fibroblasts. The Journal of Biological Chemistry, 1989, 264(13): 7147-7152.
¹⁰²³ Czuwara-Ladykowska J, Shirasaki F. Jackers P, Watson D K, Trojanowska M. Fli-1 inhibits collagen type I production in dermal fibroblasts via an Spl-dependent pathway. J Biol. Chem. 2001 Jun. 15; 276(24):20839-48.
¹⁰²⁴ Grahame R. Joint hypermobility and genetic collagen disorders: are they related? Arch Dis Child, 1999, 80: 188-191.
¹⁰²⁵ Byers P H, Duvic M, Atkinson M, Robinow M, Smith L T, Krane S M, Greally M T, Ludman M, Matalon R, Pauker S, Quanbeck D, Schwarze U. Ehlers-Danlos syndrome type VIIA and VIIB result from splice-junction mutations or genomic deletions that involve exon 6 in the COL1A1 and COL1A2 genes of type I collagen. Am J Med Genet 1997 Oct. 3; 72(1):94-105.
¹⁰²⁶ Giunta C, Superti-Furga A, Spranger S, Cole W G, Steinmann B. Ehlers-Danlos syndrome type VII: clinical features and molecular defects. J Bone Joint Surg Am 1999 February; 81(2):225-38.
¹⁰²⁷ Grahame R. How often, when and how does joint hypermobility lead to osteoarthritis? Br J Rheumatol 1989 August; 28(4):320.
¹⁰²⁸ Scott D, Bird H, Wright V. Joint laxity leading to osteoarthrosis. Rheumatol Rehabil. 1979 August; 18(3):167-9.
¹⁰²⁹ Sharma L, Lou C, Felson D T, Dunlop D D, Kirwan-Mellis G, Hayes K W, Weinrach D, Buchanan T S. Laxity in healthy and osteoarthritic knees. Arthritis Rheum 1999 May; 42(5):861-70.
¹⁰³⁰ Jonsson H, Valtysdottir S T, Kjartansson O, Brekkan A. Hypermobility associated with osteoarthritis of the thumb base: a clinical and radiological subset of hand osteoarthritis. Ann Rheum Dis 1996 August; 55(8):540-3.
¹⁰³¹ Batti'e M C, Bigos S J, Sheehy A, Wortley M D. Spinal flexibility and individual factos that influence it. Physical Therapy 1987 67(5):653-658.
¹⁰³² Cicuttini F M., Baker J R, Spector T D. The Association of Obesity with Osteoarthritis of the Hand and Knee in Women: A Twin Study. The Journal of Rheumatology, 1996,23:7, 1221-1226.
¹⁰³³ Carman W J, Sowers M, Hawthorne V M, Weissfeld L A. Obesity as a risk factor for osteoarthritis of the hand and wrist: a prospective study. Am J Epidemiol 1994 Jan. 15; 139(2):119-29.
¹⁰³⁴ Van Saase J L C M, Vandenbroucke M P, van Romunde L K J, Valkenburg H A. Osteoarthritis and Obesity in the General Population. A Relation Calling for and Explanation. The Journal of Rheumatology, 1988, 15(7): 1152-1158.
¹⁰³⁵ Ferguson K A, Love L L, Ryan C F. Effect of mandibular and tongue protrusion on upper airway size during wakefulness. Am J Respir Crit Care Med 1997 May; 155(5):1748-54.
¹⁰³⁶ Miyamoto K, Ozbek M M, Lowe A A, Sjoholm T T, Love L L, Fleetham J A, Ryan C F. Mandibular posture during sleep in patients with obstructive sleep apnoea. Arch Oral Biol 1999 August; 44(8):657-64.
¹⁰³⁷ Sellers W R, Kaelin W G Jr. Role of the retinoblastoma protein in the pathogenesis of human cancer. J Clin Oncol 1997 November; 15(11):3301-12.
¹⁰³⁸ Dou Q P, An B. RB and apoptotic cell death. Front Biosci. 1998 Apr. 6; 3:d419-30.
¹⁰³⁹ Coppola J A, Lewis B A, Cole M D. Increased retinoblastoma gene expression is associated with late stages of differentiation in many different cell types. Oncogene 1990 November; 5(11):1731-3.
¹⁰⁴⁰ Levine R A, Hopman T, Guo L, Chang M J, Johnson N. Induction of retinoblastoma gene expression during terminal growth arrest of a conditionally immortalized fetal rat lung epithelial cell line and during fetal lung maturation. Exp Cell Res. 1998 Mar. 15; 239(2):264-76.
¹⁰⁴¹ Slack R S, Hamel P A, Bladon T S, Gill R M, McBurney M W. Regulated expression of the retinoblastoma gene in differentiating embryonal carcinoma cells. Oncogene. 1993 June; 8(6):1585-91.
¹⁰⁴² Schwartz B, Avivi-Green C, Polak-Charcon S. Sodium butyrate induces retinoblastoma protein dephosphorylation, p16 expression and growth arrest of colon cancer cells. Mol Cell Biochem. 1998 November; 188(1-2):21-30.
¹⁰⁴³ Cherington V, Brown M, Paucha E, St Louis I, Spiegelman B M, Roberts T M. Separation of simian virus 40 large-T-antigen-transforming and origin-binding functions from the ability to block differentiation. Mol Cell Biol. 1988 March; 8(3):1380-4.
¹⁰⁴⁴ Cepko C L, Roberts B E, Mulligan R C. Construction and applications of a highly transmissible murine retrovirus shuttle vector. Cell. 1984 July; 37(3):1053-62.
¹⁰⁴⁵ Higgins C, Chatterjee S, Cherington V. The block of adipocyte differentiation by a C-terminally truncated, but not by full-length, simian virus 40 large tumor antigen is dependent on an intact retinoblastoma susceptibility protein family binding domain. J. Virol. 1996 February; 70(2):745-52.
¹⁰⁴⁶ Armelin H A, Armelin M C, Kelly K, Stewart T, Leder P, Cochran B H, Stiles C D. Functional role for c-myc in mitogenic response to platelet-derived growth factor. Nature. 1984 Aug. 23-29; 310(5979):655-60.
¹⁰⁴⁷Awazu S, Nakata K, Hida D, Sakamoto T, Nagata K, Ishii N, Kanematsu T. Stable transfection of retinoblastoma gene promotes contact inhibition of cell growth and hepatocyte nuclear factor-1-mediated transcription in human hepatoma cells. Biochem Biophys Res Commun. 1998 Nov. 9; 252(1):269-73.
¹⁰⁴⁸ Choi S J, Oba Y, Gazitt Y, Alsina M, Cruz J, Anderson J, Roodman G D. Antisense inhibition of macrophage inflammatory protein 1-alpha blocks bone destruction in a model of myeloma bone disease. J Clin Invest. 2001 December; 108(12):1833-41.
¹⁰⁴⁹ Hu Z. Garen A. Targeting tissue factor on tumor vascular endothelial cells and tumor cells for immunotherapy in mouse models of prostatic cancer. Proc Natl Acad Sci USA. 2001 Oct. 9; 98(21):12180-5.
¹⁰⁵⁰ Kuhn S T. The Structure of Scientific Revolution. The University of Chicago Press. 1962.
¹⁰⁵¹ Jacque J M, Fernandez B, Arenzana-Seisdedos F, Thomas D, Baleux F, Virelizier J L, Bachelerie F. Permanent occupancy of the human immunodeficiency virus type I enhancer by NF-kappa B is needed for persistent viral replication in monocytes. J. Virol. 1996 May; 70(5):2930-8.
¹⁰⁵² Garrity P A, Chen D, Rothenberg E V, Wold B J. Interleukin-2 transcription is regulated in vivo at the level of coordinated binding of both constitutive and regulated factors. Mol Cell Biol. 1994 March; 14(3):2159-69.
¹⁰⁵³ Szymczyna B R, Arrowsmith C H. DNA binding specificity studies of four ETS proteins support an indirect read-out mechanism of protein-DNA recognition. J Biol. Chem. 2000 Sep. 15; 275(37):28363-70.
¹⁰⁵⁴ Atlas E, Stramwasser M, Whiskin K, Mueller C R. GA-binding protein alpha/beta is a critical regulator of the BRCA1 promoter. Oncogene 2000 Apr. 6; 19(15):1933-40.
¹⁰⁵⁵ Thompson M E, Jensen R A, Obermiller P S, Page D L, Holt J T. Decreased expression of BRCA1 accelerates growth and is often present during sporadic breast cancer progression. Nat Genet 1995 April; 9(4):444-50.
¹⁰⁵⁶ Rao V N, Shao N, Ahmad M, Reddy E S. Antisense RNA to the putative tumor suppressor gene BRCA1 transforms mouse fibroblasts. Oncogene. 1996 Feb. 1; 12(3):523-8.
¹⁰⁵⁷ Holt J T, Thompson M E, Szabo C, Robinson-Benion C, Arteaga C L, King M C, Jensen R A. Growth retardation and tumour inhibition by BRCA1. Nat Genet. 1996 March; 12(3):298-302.
¹⁰⁵⁸ Tait D L, Obermiller P S, Redlin-Frazier S, Jensen R A, Welcsh P, Dann J, King M C, Johnson D H, Holt J T. A phase I trial of retroviral BRCA1sv gene therapy in ovarian cancer. Clin Cancer Res. 1997 November; 3(11):1959-68.
¹⁰⁵⁹ Russell P A, Pharoab P D, De Foy K, Ramus S J, Symmonds I, Wilson A, Scott I, Ponder B A, Gayther S A. Frequent loss of BRCA1 mRNA and protein expression in sporadic ovarian cancers. Int J Cancer. 2000 August; 87(3):317-321.
¹⁰⁶⁰ Rio P G, Maurizis J C, Peffault de Latour M, Bignon Y J, Bernard-Gallon D J. Quantification of BRCA1 protein in sporadic breast carcinoma with or without loss of heterozygosity of the BRCA1 gene. Int J Cancer 1999 Mar. 15; 80(6):823-6.
¹⁰⁶¹ Rice J C, Massey-Brown K S, Futscher B W. Aberrant methylation of the BRCA1 CpG island promoter is associated with decreased BRCA1 mRNA in sporadic breast cancer cells. Oncogene. 1998 Oct. 8; 17(14):1807-12.
¹⁰⁶² Magdinier F, Ribieras S, Lenoir G M, Frappart L, Dante R. Down-regulation of BRCA1 in human sporadic breast cancer; analysis of DNA methylation patterns of the putative promoter region. Oncogene. 1998 Dec. 17; 17(24):3169-76.
¹⁰⁶³ Ozcelik H, To M D, Couture J, Bull S B, Andrulis I L. Preferential allelic expression can lead to decreased expression of BRCA1 in sporadic breast cancers. Int J Cancer 1998 Jul. 3; 77(1):1-6.
¹⁰⁶⁴ Futreal P A, Liu Q, Shattuck-Eidens D, Cochran C, Harshman K, Tavtigian S, Bennett L M, Haugen-Strano A, Swensen J, Miki Y, et al. BRCA1 mutations in primary breast and ovarian carcinomas. Science 1994 Oct. 7; 266(5182): 120-2.
¹⁰⁶⁵ Merajver S D, Pham T M, Caduff R F, Chen M, Poy E L, Cooney K A, Weber B L, Collins F S, Johnston C, Frank T S. Somatic mutations in the BRCA1 gene in sporadic ovarian tumours. Nat Genet. 1995 April; 9(4):439-43.
¹⁰⁶⁶ Catteau A, Harris W H, Xu C F, Solomon E. Methylation of the BRCA1 promoter region in sporadic breast and ovarian cancer: correlation with disease characteristics. Oncogene. 1999 Mar. 18; 18(11): 1957-65.
¹⁰⁶⁷ Dobrovic A, Simpfendorfer D. Methylation of the BRCA1 gene in sporadic breast cancer. Cancer Res. 1997 Aug. 15; 57(16):3347-50.
¹⁰⁶⁸ Li X R, Chong A S, Wu J, Roebuck K A, Kumar A, Parrillo J E, Rapp U R, Kimberly R P, Williams J W, Xu X. Transcriptional regulation of Fas gene expression by GA-binding protein and AP-1 in T-cell antigen receptor CD3 complex-stimulated T-cells. J Biol. Chem. 1999 Dec. 3; 274(49):35203-10.
¹⁰⁶⁹ Davidson W F, Giese T, Fredrickson T N. Spontaneous development of plasmacytoid tumors in mice with defective Fas-Fas ligand interactions. J Exp Med. 1998 Jun. 1; 187(11):1825-38.
¹⁰⁷⁰ Drappa J, Vaishnaw A K, Sullivan K E, Chu J L, Elkon K B. Fas gene mutations in the Canale-Smith syndrome, an inherited lymphoproliferative disorder associated with autoimmunity. N Engl J. Med. 1996 Nov. 28; 335(22):1643-9.
¹⁰⁷¹ Keane M M, Ettenberg S A, Lowrey G A, Russell E K, Lipkowitz S. Fas expression and function in normal and malignant breast cell lines. Cancer Res. 1996 Oct. 15; 56(20):4791-8.
¹⁰⁷² Gratas C, Tohma Y, Barnas C, Taniere P, Hainaut P, Ohgaki H. Up-regulation of Pas (APO-1/CD95) ligand and down-regulation of Fas expression in human esophageal cancer. Cancer Res. 1998 May 15; 58(10):2057-62.
¹⁰⁷³ Strand S, Hofmann W J, Hug H, Muller M, Otto G, Strand D, Mariani S M, Stremmel W, Krammer P H, Galle P R. Lymphocyte apoptosis induced by CD95 (APO-1/Fas) ligand-expressing tumor cells—a mechanism of immune evasion? Nat Med. 1996 December; 2(12):1361-6.
¹⁰⁷⁴ Moller P, Koretz K, Leithauser F, Bruderlein S, Henne C, Quentmeier A, Krammer P H. Expression of APO-1 (CD95), a member of the NGF/TNF receptor superfamily, in normal and neoplastic colon epithelium. Int J Cancer. 1994 May 1; 57(3):371-7.
¹⁰⁷⁵ Leithauser F, Dhein J, Mechtersheimer G, Koretz K, Bruderlein S, Henne C, Schmidt A, Debatin K M, Krammer P H, Moller P. Constitutive and induced expression of APO-1, a new member of the nerve growth factor/tumor necrosis factor receptor superfamily, in normal and neoplastic cells. Lab Invest. 1993 October; 69(4):415-29.
¹⁰⁷⁶ Das H, Koizumi T, Sugimoto T, Chakraborty S, Ichimura T, Hasegawa K, Nishimura R. Quantitation of Fas and Fas ligand gene expression in human ovarian, cervical and endometrial carcinomas using real-time quantitative RT-PCR. Br J Cancer. 2000 May; 82(10):1682-8.
¹⁰⁷⁷ Butler L M, Hewett P J, Butler W J, Cowled P A. Down-regulation of Fas gene expression in colon cancer is not a result of allelic loss or gene rearrangement. Br J Cancer. 1998 May; 77(9):1454-9.
¹⁰⁷⁸ Bertoni F, Conconi A, Carobbio S, Realini C, Codegoni A M, Zucca E, Cavalli F. Analysis of Fas/CD95 gene somatic mutations in ovarian cancer cell lines. Int J Cancer. 2000 May 1; 86(3):450.
¹⁰⁷⁹ Lee S H, Shin M S, Park W S, Kim S Y, Kim H S, Han J Y, Park G S, Dong S M, Pi J H, Kim C S, Kim S H, Lee J Y, Yoo N J. Alterations of Fas (Apo-1/CD95) gene in non-small cell lung cancer. Oncogene. 1999 Jun. 24; 18(25):3754-60.
¹⁰⁸⁰ Lee S H, Shin M S, Park W S, Kim S Y, Dong S M, Pi J H, Lee H K, Kim H S, Jang J J, Kim C S, Kim S H, Lee JY, Yoo N J. Alterations of Fas (APO-1/CD95) gene in transitional cell carcinomas of urinary bladder. Cancer Res 1999 Jul. 1; 59(13):3068-72.
¹⁰⁸¹ Shin M S, Park W S, Kim S Y, Kim H S, Kang S J, Song K Y, Park J Y, Dong S M, Pi J H, Oh R R, Lee J Y, Yoo N J, Lee S H. Alterations of Fas (Apo-1/CD95) gene in cutaneous malignant melanoma. Am J Pathol 1999 June; 154(6):1785-91.
¹⁰⁸² Butler L M, Dobrovic A, Bianco T, Cowled P A. Promoter region methylation does not account for the frequent loss of expression of the Fas gene in colorectal carcinoma. Br J Cancer. 2000 January; 82(1):131-5.
¹⁰⁸³ Watson J D, Crick F H C. Molecular Structure of Nucleic Acid. Nature, 1953, 737-738.
¹⁰⁸⁴ Ohta S, Wada H, Nakazaki T. Maeda Y, Nobori T, Shiku H. Nakamura S, Nagakawa O, Furuya Y, Fuse H. Expression of tissue factor is associated with clinical features and angiogenesis in prostate cancer. Anticancer Res. 2002 September-October; 22(5):2991-6.
¹⁰⁸⁵ Guan M, Su B, Lu Y. Quantitative reverse transcription-PCR measurement of tissue factor mRNA in glioma. Mol Biotechnol. 2002 February; 20(2): 123-9.
¹⁰⁸⁶ Nakasaki T, Wada H, Shigemori C, Miki C, Gabazza E C, Nobori T, Nakamura S, Shiku H. Expression of tissue factor and vascular endothelial growth factor is associated with angiogenesis in colorectal cancer. Am J Hematol. 2002 April; 69(4):247-54.
¹⁰⁸⁷ Sawada M, Miyake S, Ohdama S, Matsubara O, Masuda S, Yakumaru K, Yoshizawa Y. Expression of tissue factor in non-small-cell lung cancers and its relation to metastasis. Br J Cancer. 1999 February; 79(3-4):472-7.
¹⁰⁸⁸ Shigemori C, Wada H, Matsumoto K, Shiku H, Nakamura S, Suzuki H. Tissue factor expression and metastatic potential of colorectal cancer. Thromb Haemost. 1998 December; 80(6):894-8.
¹⁰⁸⁹ Mueller B M, Reisfeld R A, Edgington T S, Ruf W. Expression of tissue factor by melanoma cells promotes efficient hematogenous metastasis. Proc Natl Acad Sci USA. 1992 Dec. 15; 89(24):11832-6.
¹⁰⁹⁰ Adamson A S, Francis J L, Witherow R O, Snell M E. Urinary tissue factor levels in prostatic carcinoma: a potential marker of metastatic spread? Br J Urol. 1993 May; 71(5):587-92.
¹⁰⁹¹ Kataoka H, Uchino H, Asada Y, Hatakeyama K, Nabeshima K, Sumiyoshi A, Koono M. Analysis of tissue factor and tissue factor pathway inhibitor expression in human colorectal carcinoma cell lines and metastatic sublines to the liver. Int J Cancer. 1997 Sep. 4; 72(5):878-84.
¹⁰⁹² Sturm U, Luther T, Albrecht S, Flossel C, Grossmann H. Muller M. Immunohistological detection of tissue factor in normal and abnormal human mammary glands using monoclonal antibodies. Virchows Arch A Pathol Anat Histopathol. 1992; 421(2):79-86.
¹⁰⁹³ Hu T, Bach R R, Horton R, Konigsberg W U, Todd M B. Procoagulant activity in cancer cells is dependent on tissue factor expression. Oncol Res. 1994; 6(7):321-7.
¹⁰⁹⁴ Lee A Y. Cancer and thromboembolic disease: pathogenic mechanisms. Cancer Treat Rev. 2002 June; 28(3):137-40. Review.
¹⁰⁹⁵ Sampson M T, Kakkar A K. Coagulation proteases and human cancer. Biochem Soc Trans. 2002 April; 30(2):201-7. Review.
¹⁰⁹⁶ Gale A J, Gordon S G. Update on tumor cell procoagulant factors. Acta Haematol. 2001; 106(1-2):25-32. Review.
¹⁰⁹⁷ Rickles F R, Shoji M, Abe K. The role of the hemostatic system in tumor growth, metastasis, and angiogenesis: tissue factor is a bifunctional molecule capable of inducing both fibrin deposition and angiogenesis in cancer. Int J Hematol. 2001 February; 73(2):145-50. Review.
¹⁰⁹⁸ Lwaleed B A, Bass P S, Cooper A J. The biology and tumour-related properties of monocyte tissue factor. J Pathol. 2001 January; 193(1):3-12. Review.
¹⁰⁹⁹ Ruf W, Fischer E G, Huang H Y, Miyagi Y, Ott I, Riewald M, Mueller B M. Diverse functions of protease receptor tissue factor in inflammation and metastasis. Immunol Res. 2000; 21(2-3):289-92.
¹¹⁰⁰ Schwartz J D, Simantov R. Thrombosis and malignancy: pathogenesis and prevention. In Vivo. 1998 November-December; 12(6):619-24.
¹¹⁰¹ Kakkar A K, Chinswangwatanakul V, Lemoine N R, Tebbutt S, Williamson R C. Role of tissue factor expression on tumour cell invasion and growth of experimental pancreatic adenocarcinoma. Br J. Surg. 1999 July; 86(7):890-4.
¹¹⁰² Bromberg M E, Konigsberg W H, Madison J F, Pawashe A, Garen A. Tissue factor promotes melanoma metastasis by a pathway independent of blood coagulation. Proc Natl Acad Sci USA. 1995 Aug. 29; 92(18):8205-9.
¹¹⁰³ Song X, Wang B, Bromberg M, Hu Z, Konigsberg W, Garen A. Retroviral-mediated transmission of a mouse VL30 RNA to human melanoma cells promotes metastasis in an immunodeficient mouse model. Proc Natl Acad Sci USA. 2002 Apr. 30; 99(9):6269-73.
¹¹⁰⁴ Pribnow D, Chen S L, Zhang Y, Magun B E. Complex interactions with direct repeats of a mitogen-responsive VL30 enhancer. Biochim Biophys Acta. 1996 Jun. 3; 1307(1):55-65.
¹¹⁰⁵ Rodland K D, Pribnow D, Lenormand P, Chen S L, Magun B E. Characterization of a unique enhancer element responsive to cyclic adenosine 3′,5′-monophosphate and elevated calcium. Mol Endocrinol. 1993 June; 7(6):787-96.
¹¹⁰⁶ Rotman G, Itin A, Keshet E. Promoter and enhancer activities of long terminal repeats associated with cellular retrovirus-like (VL30) elements. Nucleic Acids Res. 1986 Jan. 24; 14(2):645-58.
¹¹⁰⁷ zur Hausen H. Viruses in human cancers. Eur J Cancer. 1999 August; 35(8): 1174-81.
¹¹⁰⁸ Hoppe-Seyler F, Butz K. Human tumor viruses. Anticancer Res. 1999 November-December; 19(6A):4747-58.
¹¹⁰⁹ Bonnet M, Guinebretiere J M, Kremmer E, Grunewald V, Benhamou E, Contesso G, Joab I. Detection of Epstein-Barr virus in invasive breast cancers. J Natl Cancer Inst. 1999 Aug. 18; 91(16): 1376-81.
¹¹¹⁰ Labrecque L G, Barnes D M, Fentiman I S, Griffin B E. Epstein-Barr virus in epithelial cell tumors: a breast cancer study. Cancer Res. 1995 Jan. 1; 55(1):39-45.
¹¹¹¹ Magrath I, Bhatia K. Breast cancer: a new Epstein-Barr virus-associated disease? J Natl Cancer Inst. 1999 Aug. 18; 91(16).1349-50.
¹¹¹² Park K, Kim S J, Bang Y J, Park J G, Kim N K, Roberts A B, Sporn M B. Genetic changes in the transforming growth factor beta (TGF-beta) type II receptor gene in human gastric cancer cells: correlation with sensitivity to growth inhibition by TGF-beta. Proc Natl Acad Sci USA. 1994 Sep. 13; 91(19):8772-6.
¹¹¹³ Myeroff L L, Parsons R, Kim S J, Hedrick L, Cho K R, Orth K, Mathis M, Kinzler K W, Lutterbaugh J, Park K, et al. A transforming growth factor beta receptor type II gene mutation common in colon and gastric but rare in endometrial cancers with microsatellite instability. Cancer Res. 1995 Dec. 1; 55(23):5545-7.
¹¹¹⁴ Markowitz S, Wang J, Myeroff L, Parsons R, Sun L, Lutterbaugh J, Fan R S, Zborowska E, Kinzler K W, Vogelstein B, et al. Inactivation of the type II TGF-beta receptor in colon cancer cells with microsatellite instability. Science. 1995Jun. 2; 268(5215):1336-8.
¹¹¹⁵ Vossen R C, Persoons M C, Slobbe-van Drunen M E, Bruggeman C A, van Dam-Mieras M C. Intracellular thiol redox status affects rat cytomegalovirus infection of vascular cells. Virus Res. 1997 May; 48(2):173-83.
¹¹¹⁶ Scholz M, Cinatl J, Gross V, Vogel J U, Blaheta R A, Freisleben H J, Markus B H, Doerr H W. Impact of oxidative stress on human cytomegalovirus replication and on cytokine-mediated stimulation of endothelial cells. Transplantation. 1996 Jun. 27; 61(12): 1763-70.
¹¹¹⁷ Ranjan D, Siquijor A, Johnston T D, Wu G, Nagabhuskahn M. The effect of curcumin on human B-cell immortalization by Epstein-Barr virus. Am Surg. 1998 January; 64(1):47-51; discussion 51-2.
¹¹¹⁸ Nakamura Y, Kawamoto N, Ohto Y, Torikai K, Murakami A, Ohigashi H. A diacetylenic spiroketal enol ether epoxide, AL-1, from Artemisia lactiflora inhibits 12-O-tetradecanoylphorbol-13-acetate-induced tumor promotion possibly by suppression of oxidative stress. Cancer Lett. 1999 Jun. 1; 140(1-2):37-45.
¹¹¹⁹ Allard J P, Aghdassi E, Chau J, Salit I, Walmsley S. Oxidative stress and plasma antioxidant micronutrients in humans with HIV infection. Am J Clin Nutr 1998 January; 67(1): 143-7.
¹¹²⁰ Allard J P, Aghdassi E, Chau J, Tam C, Kovacs C M, Salit I E, Walmsley S L. Effects of vitamin E and C supplementation on oxidative stress and viral load in HIV-infected subjects. AIDS. 1998 Sep. 10; 12(13):1653-9.
¹¹²¹ Helen A, Krishnakumar K, Vijayammal P L, Augusti K T. Antioxidant effect of onion oil (Allium cepa. Linn) on the damages induced by nicotine in rats as compared to alpha-tocopherol. Toxicol Lett. 2000 Jul. 27; 116(1-2):61-8.
¹¹²² Yildiz D, Liu Y S, Ercal N, Armstrong D W. Comparison of pure nicotine- and smokeless tobacco extract-induced toxicities and oxidative stress. Arch Environ Contam Toxicol. 1999 November; 37(4):434-9.
¹¹²³ Yildiz D, Ercal N, Armstrong D W. Nicotine enantiomers and oxidative stress. Toxicology 1998 Sep. 15; 130(2-3):155-65.
¹¹²⁴ Afaq F, Abidi P, Rahman Q. N-acetyl L-cysteine attenuates oxidant-mediated toxicity induced by chrysotile fibers. Toxicol Lett. 2000 Sep. 30; 117(1-2):53-60.
¹¹²⁵ Abidi P, Afaq F, Arif J M, Lohani M, Rahman Q. Chrysotile-mediated imbalance in the glutathione redox system in the development of pulmonary injury. Toxicol Lett 1999 May 20; 106(1):31-9.
¹¹²⁶ Liu W, Ernst J D, Courtney Broaddus V. Phagocytosis of crocidolite asbestos induces oxidative stress, DNA damage, and apoptosis in mesothelial cells. Am J Respir Cell Mol. Biol. 2000 September; 23(3):371-8.
¹¹²⁷ Marczynski B, Kraus T, Rozynek P, Raithel H J, Baur X. Association between 8-hydroxy-2′-deoxyguanosine levels in DNA of workers highly exposed to asbestos and their clinical data, occupational and non-occupational confounding factors, and cancer. Mutat Res. 2000 Jul. 10; 468(2):203-12.
¹¹²⁸ Marczynski B, Rozynek P, Kraus T, Schlosser S, Raithel H J, Baur X. Levels of 8-hydroxy-2′-deoxyguanosine in DNA of white blood cells from workers highly exposed to asbestos in Germany. Mutat Res. 2000 Jul. 10; 468(2):195-202.
¹¹²⁹ Fisher C E, Rossi A G, Shaw J, Beswick P H, Donaldson K. Release of TNFalpha in response to SiC fibres: differential effects in rodent and human primary macrophages, and in macrophage-like cell lines. Toxicol In Vitro. 2000 February; 14(1):25-31.
¹¹³⁰ Brown D M, Beswick P H, Bell K S, Donaldson K. Depletion of glutathione and ascorbate in lung lining fluid by respirable fibres. Ann Occup Hyg. 2000 March; 44(2j: 01 i-8.
¹¹³¹ He H, Wang X, Gorospe M, Holbrook N J, Trush M A. Phorbol ester-induced mononuclear cell differentiation is blocked by the mitogen-activated protein kinase kinase (MEK) inhibitor PD98059. Cell Growth Differ 1999 May; 10(5):307-15.
¹¹³² Mizokami A, Yeh S Y, Chang C. Identification of 3′,5′-cyclic adenosine monophosphate response element and other cis-acting elements in the human androgen receptor gene promoter. Mol Endocrinol. 1994 January; 8(1):77-88.
¹¹³³ Ree A H, Hansson V, Walaas S I, Eskild W, Tasken K A. Calcium/phospholipid-dependent protein kinases in rat Sertoli cells: regulation of androgen receptor messenger ribonucleic acid. Biol Reprod. 1999 May; 60(5): 1257-62.
¹¹³⁴ Su Y Q, Rubinstein S, Luria A, Lax Y, Breitbart H. Involvement of MEK-mitogen-activated protein kinase pathway in follicle-stimulating hormone-induced but not spontaneous meiotic resumption of mouse oocytes. Biol Reprod. 2001 August; 65(2):358-65.
¹¹³⁵ Seger R, Hanoch T, Rosenberg R, Dantes A, Merz W E, Strauss J F 3rd, Amsterdam A. The ERK signaling cascade inhibits gonadotropin-stimulated steroidogenesis. J Biol. Chem. 2001 Apr. 27; 276(17):13957-64.
¹¹³⁶ Babu P S, Krishnamurthy H, Chedrese P J, Sairam M R. Activation of extracellular-regulated kinase pathways in ovarian granulosa cells by the novel growth factor type I follicle-stimulating hormone receptor. Role in hormone signaling and cell proliferation. J Biol. Chem. 2000 Sep. 8; 275(36):27615-26.
¹¹³⁷ Das S, Maizels E T, DeManno D, St Clair E, Adam S A, Hunzicker-Dunn M. A stimulatory role of cyclic adenosine 3′,5′-monophosphate in follicle-stimulating hormone-activated mitogen-activated protein kinase signaling pathway in rat ovarian granulosa cells. Endocrinology. 1996 March; 137(3):967-74.
¹¹³⁸ Cameron M R, Foster J S, Bukovsky A, Wimalasena J. Activation of mitogen-activated protein kinases by gonadotropins and cyclic adenosine 5′-monophosphates in porcine granulosa cells. Biol Reprod. 1996 July; 55(1): 111-9.
¹¹³⁹ Blok L J, Hoogerbrugge J W, Themmen A P, Baarends W M, Post M, Grootegoed J A. Transient down-regulation of androgen receptor messenger ribonucleic acid (mRNA) expression in Sertoli cells by follicle-stimulating hormone is followed by up-regulation of androgen receptor mRNA and protein. Endocrinology. 1992 September; 131(3):1343-9.
¹¹⁴⁰ Crepieux P, Marion S, Martinat N, Fafeur V, Vern Y L, Kerboeuf D, Guillou F, Reiter E. The ERK-dependent signalling is stage-specifically modulated by FSH, during primary Sertoli cell maturation. Oncogene. 2001 Aug. 2; 20(34):4696-709.
¹¹⁴¹ Wilson D J, Alessandrini A, Budd R C. MEK1 activation rescues Jurkat T-cells from Fas-induced apoptosis. Cell Immunol. 1999 May 25; 194(1):67-77.
¹¹⁴² Li Y Q, Hii C S, Costabile M, Gob D, Der C J, Ferrante A. Regulation of lymphotoxin production by the p21ras-raf-MEK-ERK cascade in PHA/PMA-stimulated Jurka T cells. J. Immunol. 1999 Mar. 15; 162(6):3316-20.
¹¹⁴³ Franklin R A, Atherfold P A, McCubrey J A. Calcium-induced ERK activation in human T lymphocytes occurs via p56(Lck) and CaM-kinase. Mol Immunol. 2000 August; 37(11):675-83.
¹¹⁴⁴ Atherfold P A, Norris M S, Robinson P J, Gelfand E W, Franklin R A. Calcium-induced ERK activation in human T lymphocytes. Mol Immunol. 1999 June; 36(8):543-9.
¹¹⁴⁵ Zhou Z, Speiser P W. Regulation of HSD17B1 and SRD5A1 in lymphocytes. Mol Genet Metab. 1999 November; 68(3):410-7.
¹¹⁴⁶ Herzog N K, Ramagli L S, Khorana S, Arlinghaus R B. Evidence for somatic cell expression of the c-mos protein [corrected]. Oncogene. 1989 November; 4(11):1307-15.
¹¹⁴⁷ Verlhac M H, Lefebvre C, Kubiak J Z, Umbhauer M, Rassinier P, Colledge W, Maro B. Mos activates MAP kinase in mouse oocytes through two opposite pathways. EMBO J. 2000 Nov. 15; 19(22):6065-74.
¹¹⁴⁸ Hochegger H, Klotzbucher A, Kirk J, Howell M, le Guellec K, Fletcher K, Duncan T, Sohail M, Hunt T. New B-type cyclin synthesis is required between meiosis I and II during Xenopus oocyte maturation. Development. 2001 October; 128(19):3795-807.
¹¹⁴⁹ Moos J, Kopf G S, Schultz R M. Cycloheximide-7induced activation of mouse eggs: effects on cdc2/cyclin B and MAP kinase activities. J Cell Sci. 1996 April; 109 (Pt 4):739-48.
¹¹⁵⁰ Sasaki K, Chiba K. Fertilization blocks apoptosis of starfish eggs by inactivation of the MAP kinase pathway. Dev Biol. 2001 Sep. 1; 237(1): 18-28.
¹¹⁵¹ Yen A, Norman A W, Varvayanis S. Nongenomic vitamin D3 analogs activating ERK2 in HL-60 cells show that retinoic acid-induced differentiation and cell cycle arrest require early concurrent MAPK and RAR and RXR activation. In Vitro Cell Dev Biol Anim. 2001 February; 37(2):93-9.
¹¹⁵² Wang X, Studzinski G P. Activation of extracellular signal-regulated kinases (ERKs) defines the first phase of 1,25-dihydroxyvitamin D3-induced differentiation of HL60 cells. J Cell Biochem. 2001; 80(4):471-82.
¹¹⁵³ Hong H Y, Varvayanis S, Yen A. Retinoic acid causes MEK-dependent RAF phosphorylation through RARalpha plus RXR activation in HL-60 cells: Differentiation. 2001 August; 68(1):55-66.
¹¹⁵⁴ Oeth P, Yao J, Fan S T, Mackman N. Retinoic acid selectively inhibits lipopolysaccharide induction of tissue factor gene expression in human monocytes. Blood. 1998 Apr. 15; 91(8):2857-65.
¹¹⁵⁵ Blok L J, Themmen A P, Peters A H, Trapman J, Baarends W M, Hoogerbrugge J W, Grootegoed J A. Transcriptional regulation of androgen receptor gene expression in Sertoli cells and other cell types. Mol Cell Endocrinol. 1992 October; 88(1-3):153-64.
¹¹⁵⁶ Willis S A, Nisen P D. Differential induction of the mitogen-activated protein kinase pathway by bacterial lipopolysaccharide in cultured monocytes and astrocytes. Biochem J. 1996 Jan. 15; 313 (Pt 2):519-24.
¹¹⁵⁷ Durando M M, Meier K E, Cook J A. Endotoxin activation of mitogen-activated protein kinase in THP-1 cells; diminished activation following endotoxindesensitization. J Leukoc Biol. 1998 August; 64(2):259-64.
¹¹⁵⁸ Takane K K, McPhaul M J. Functional analysis of the human androgen receptor promoter. Mol Cell Endocrinol. 1996 May 17; 119(1):83-93.
¹¹⁵⁹ Brown J W, Kesler C T, Neary T, Fishman L M. Effects of androgens and estrogens and catechol and methoxy-estrogen derivatives on mitogen-activated protein kinase (ERK(1,2)) activity in SW-13 human adrenal carcinoma cells. Horm Metab Res. 2001 March; 33(3):127-30.
¹¹⁶⁰ Peterziel H, Mink S, Schonert A, Becker M, Klocker H, Cato A C. Rapid signalling by androgen receptor in prostate cancer cells. Oncogene. 1999 Nov. 4; 18(46):6322-9.
¹¹⁶¹ Zhu X, Li H, Liu J P, Funder J W. Androgen stimulates mitogen-activated protein kinase in human breast cancer cells. Mol Cell Endocrinol. 1999 Jun. 25; 152(1-2):199-206.
¹¹⁶² Guo C, Luttrell L M, Price D T. Mitogenic signaling in androgen sensitive and insensitive prostate cancer cell lines. J Urol. 2000 March; 163(3):1027-32.
¹¹⁶³ Kue P F, Daaka Y. Essential role for G proteins in prostate cancer cell growth and signaling. J Urol. 2000 December; 164(6):2162-7.
¹¹⁶⁴ Chen T, Cho R W, Stork P J, Weber M J. Elevation of cyclic adenosine 3′,5′-monophosphate potentiates activation of mitogen-activated protein kinase by growth factors in LNCaP prostate cancer cells. Cancer Res. 1999 Jan. 1; 59(1):213-8.
¹¹⁶⁵ Putz T, Culig Z, Eder I E, Nessler-Menardi C, Bartsch G, Grunicke H, Uberall F, Klocker H. Epidermal growth factor (EGF) receptor blockade inhibits the action of EGF, insulin-like growth factor I, and a protein kinase A activator on the mitogen-activated protein kinase pathway in prostate cancer cell lines. Cancer Res. 1999 Jan. 1; 59(1):227-33. Magi-Galluzzi C, Montironi R, Cangi M G, Wishnow K, Loda M. Mitogen-activated protein kinases and apoptosis in PIN. Virchows Arch. 1998 May; 432(5):407-13.
¹¹⁶⁷ Mizokami A, Saiga H, Matsui T, Mita T, Sugita A. Regulation of androgen receptor by androgen and epidermal growth factor in a human prostatic cancer cell line, LNCaP. Endocrinol Jpn. 1992 June; 39(3):235-43.
¹¹⁶⁸ Yeap B B, Krueger R G, Leedman P J. Differential posttranscriptional regulation of androgen receptor gene expression by androgen in prostate and breast cancer cells. Endocrinology. 1999 July; 140(7):3282-91.
¹¹⁶⁹ Quarmby V E, Yarbrough W G, Lubahn D B, French F S, Wilson E M. Autologous down-regulation of androgen receptor messenger ribonucleic acid. Mol Endocrinol. 1990 January; 4(1):22-8.
¹¹⁷⁰ Henttu P, Vihko P. Growth factor regulation of gene expression in the human prostatic carcinoma cell line LNCaP. Cancer Res. 1993 Mar. 1; 53(5):1051-8.
¹¹⁷¹ Mizokami A, Gotoh A, Yamada H, Keller E T, Matsumoto T. Tumor necrosis factor-alpha represses androgen sensitivity in the LNCaP prostate cancer cell line. J Urol. 2000 September; 164(3 Pt 1):800-5.
¹¹⁷² Sokoloff M H, Tso C L, Kaboo R, Taneja S. Pang S, deKernion J B, Belldegrun A S. In vitro modulation of tumor progression-associated properties of hormone refractory prostate carcinoma cell lines by cytokines. Cancer. 1996 May 1; 77(9): 1862-72.
¹¹⁷³ Segawa N, Nakamura M, Nakamura Y, Mori I, Katsuoka Y, Kakudo K. Phosphorylation of mitogen-activated protein kinase is inhibited by calcitonin in DU145 prostate cancer cells. Cancer Res. 2001 Aug. 15; 61(16):6060-3.
¹¹⁷⁴ Kumar M V, Jones E A, Felts S J, Blexrud M D, Grossmann M E, Blok L J, Schmidt L J, Tindall D J. Characterization of a TPA-response element in the 5′-flanking region of the androgen receptor gene. J Androl. 1998 September-October; 19(5):595-602.
¹¹⁷⁵ Lin D L, Whitney M C, Yao Z, Keller E T. Interleukin-6 induces androgen responsiveness in prostate cancer cells through up-regulation of androgen receptor expression. Clin Cancer Res. 2001 June; 7(6):1773-81.
¹¹⁷⁶ Chen T, Wang L H, Farrar W L. Interleukin 6 activates androgen receptor-mediated gene expression through a signal transducer and activator of transcription 3-dependent pathway in LNCaP prostate cancer cells. Cancer Res. 2000 Apr. 15; 60(8):2132-5.
¹¹⁷⁷ Diani A R, Mills C J. Immunocytochemical localization of androgen receptors in the scalp of the stumptail macaque monkey, a model of androgenetic alopecia. J Invest Dermatol. 1994 April; 102(4):511-4.
¹¹⁷⁸ Ando Y, Yamaguchi Y, Hamada K, Yoshikawa K, Itami S. Expression of mRNA for androgen receptor, 5alpha-reductase and 17beta-hydroxysteroid dehydrogenase in human dermal papilla cells. Br J. Dermatol. 1999 November; 141 (5):840-5.
¹¹⁷⁹ Kiesewetter F, Arai A, Schell H. Sex hormones and antiandrogens influence in vitro growth of dermal papilla cells and outer root sheath keratinocytes of human hair follicles. J Invest Dermatol. 1993 July; 101(1 Suppl):98S-105S.
¹¹⁸⁰ Obana N, Chang C, Uno H. Inhibition of hair growth by testosterone in the presence of dermal papilla cells from the frontal bald scalp of the postpubertal stumptailed macaque. Endocrinology. 1997 January; 138(1):356-61.
¹¹⁸¹ Choudhry R, Hodgins M B, Van der Kwast T H, Brinkmann A O, Boersma W J. Localization of androgen receptors in human skin by immunohistochemistry: implications for the hormonal regulation of hair growth, sebaceous glands and sweat glands. J Endocrinol. 1992 June; 133(3):467-75.
¹¹⁸² Deplewski D, Rosenfield R L. Growth hormone and insulin-like growth factors have different effects on sebaceous cell growth and differentiation. Endocrinology. 1999 September; 140(9):4089-94.
¹¹⁸³ Krishnamurthy H, Kats R, Danilovich N, Javeshghani D, Ram Sairam M. Intercellular communication between sertoli cells and leydig cells in the absence of follicle-stimulating hormone-receptor signaling. Biol Reprod. 2001 October; 65(4):1201-7.
¹¹⁸⁴ Thiboutot D, Bayne E, Thorne J, Gilliland K, Flanagan J, Shao Q, Light J, Heim K. Immunolocalization of 5alpha-reductase isozymes in acne lesions and normal skin. Arch Dermatol. 2000 September; 136(9): 1125-9.
¹¹⁸⁵ Bayne E K, Flanagan J, Einstein M, Ayala J, Chang B, Azzolina B, Whiting D A, Mumford R A, Thiboutot D, Singer I I, Harris G. Immunohistochemical localization of types 1 and 2 5alpha-reductase in human scalp. Br J. Dermatol. 1999 September; 141(3):481-91.
¹¹⁸⁶ Chen W, Zouboulis C C, Fritsch M, Blume-Peytavi U, Kodelja V, Goerdt S, Luu-The V, Orfanos C E. Evidence of heterogeneity and quantitative differences of the type 1 5alpha-reductase expression in cultured human skin cells—evidence of its presence in melanocytes. J Invest Dermatol. 1998 January; 110(1):84-9.
¹¹⁸⁷ Chen W, Zouboulis C C, Orfanos C E. The 5 alpha-reductase system and its inhibitors. Recent development and its perspective in treating androgen-dependent skin disorders. Dermatology. 1996; 193(3): 177-84.
¹¹⁸⁸ Deplewski D, Rosenfield R L. Role of hormones in pilosebaceous unit development. Endocr Rev. 2000 August; 21(4):363-92. Review.
¹¹⁸⁹ Fritsch M, Orfanos C E, Zouboulis C C. Sebocytes are the key regulators of androgen homeostasis in human skin. J Invest Dermatol. 2001 May; 116(5):793-800.
¹¹⁹⁰ Clements G B, Jamieson F E. Reactivation of latent herpes simplex virus-1 (HSV) from mouse footpad cells demonstrated by in situ hybridization. Arch Virol. 1989; 104(1-2):95-106.
¹¹⁹¹ Moriyama K, Imayama S, Mohri S, Kurata T, Mori R. Localization of herpes simplex virus type I in sebaceous glands of mice. Arch Virol. 1992; 123(1-2): 13-27.
¹¹⁹² Okimoto M A, Fan H. Moloney murine leukemia virus infects cells of the developing hair follicle after neonatal subcutaneous inoculation in mice. J. Virol. 1999 March; 73(3):2509-16.
¹¹⁹³ Lattanand A, Johnson W C. Male pattern alopecia a histopathologic and histochemical study. J Cutan Pathol. 1975; 2(2):58-70.
¹¹⁹⁴ Puerto A M, Mallol J. Regional scalp differences of the androgenic metabolic pattern in subjects affected by male pattern baldness. Rev Esp Fisiol. 1990 September; 46(3):289-96.
¹¹⁹⁵ Giralt M, Cervello I, Nogues M R, Puerto A M, Ortin F, Argany N, Mallol J. Glutathione, glutathione S-transferase and reactive oxygen species of human scalp sebaceous glands in male pattern baldness. J Invest Dermatol. 1996 August; 107(2): 154-8.
¹¹⁹⁶ Arend W P, Malyak M, Guthridge C J, Gabay C. Interleukin-1 receptor antagonist: role in biology. Annu Rev Immunol. 1998; 16:27-55.
¹¹⁹⁷ Anttila H S, Reitamo S, Saurat J H. Interleukin I immunoreactivity in sebaceous glands. Br J. Dermatol. 1992 December; 127(6):585-8.
¹¹⁹⁸ Matsukawa A, Fukumoto T, Maeda T, Ohkawara S, Yoshinaga M. Detection and characterization of IL-1 receptor antagonist in tissues from healthy rabbits: IL-1 receptor antagonist is probably involved in health. Cytokine. 1997 May; 9(5):307-15.
¹¹⁹⁹ Kristensen M, Deleuran B, Eedy D J, Feldmann M, Breathnach S M, Brennan F M. Distribution of interleukin 1 receptor antagonist protein (IRAP), interleukin 1 receptor, and interleukin 1 alpha in normal and psoriatic skin. Decreased expression of IRAP in psoriatic lesional epidermis. Br J. Dermatol. 1992 October; 127(4):305-11.
¹²⁰⁰ Tebo J M, Datta S, Kishore R, Kolosov M, Major J A, Ohmori Y, Hamilton T A. Interleukin-1-mediated stabilization of mouse KC mRNA depends on sequences in both 5′- and 3′-untranslated regions. J Biol Chem 2000 Apr. 28; 275(17):12987-93.
¹²⁰¹ Awane M, Andres P G, Li D J, Reinecker H C. NF-kappa B-inducing kinase is a common mediator of IL-17-, TNF-alpha-, and IL-1 beta-induced chemokine promoter activation in intestinal epithelial cells. J. Immunol. 1999 May 1; 162(9):5337-44.
¹²⁰² Hybertson B M, Jepson E K, Clarke J H, Spelts R J, Repine J B. Interleukin-1 stimulates rapid release of cytokine-induced neutrophil chemoattractant (CINC) in rat lungs. Inflammation. 1996 October; 20(5):471-83.
¹²⁰³ Koh Y, Hybertson B M, Jepson E K, Cho O J, Repine J E. Cytokine-induced neutrophil chemoattractant is necessary for interleukin-1-induced lung leak in rats. J Appl Physiol 1995 August; 79(2):4728.
¹²⁰⁴ Fujimori H, Miura S, Koseki S, Hokari R, Tsuzuki Y, Komoto S, Hara Y, Suzuki H, Serizawa H. Ishii H. Intravital demonstration of modulation of T lymphocyte migration by CINC/gro in rat Peyer's patches. Digestion. 2001; 63 Suppl 1:97-102.
¹²⁰⁵ Jinquan T, Frydenberg J, Mukaida N, Bonde J, Larsen C G, Matsushima K, Thestrup-Pedersen K. Recombinant human growth-regulated oncogene-alpha induces T lymphocyte chemotaxis. A process regulated via IL-8 receptors by IFN-gamma, TNF-alpha, IL-4, IL-10, and IL-13. J Immunol 1995 Dec. 1; 155(11):5359-68.
¹²⁰⁶ Aust G, Steinert M, Boltze C, Kiessling S, Simchen C. GRO-alpha in normal and pathological thyroid tissues and its regulation in thyroid-derived cells. J Endocrinol. 2001 September; 170(3):513-20.
¹²⁰⁷ Tettelbach W, Nanney L, Ellis D, King L, Richmond A. Localization of MGSA/GRO protein in cutaneous lesions. J Cutan Pathol. 1993 June; 20(3):259-66.
¹²⁰⁸ Sueki H, Stoudemayer T, Kligman A M, Murphy G F. Quantitative and ultrastructural analysis of inflammatory infiltrates in male pattern alopecia. Acta Dern Venereol. 1999 September; 79(5):347-50.
¹²⁰⁹ Jaworsky C, Kligman A M, Murphy G F. Characterization of inflammatory infiltrates in male pattern alopecia: implications for pathogenesis. Br J. Dermatol. 1992 September; 127(3):239-46.
¹²¹⁰ Hofin R, Happle R, Paus R. Elements of the interleukin-1 signaling system show hair cycle-dependent gene expression in murine skin. Eur J Dermatol 1998 October-November; 8(7):475-7.
¹²¹¹ Philpott M P, Sanders D A, Bowen J, Kealey T. Effects of interleukins, colony-stimulating factor and tumour necrosis factor on human hair follicle growth in vitro: a possible role for interleukin-1 and tumour necrosis factor-alpha in alopecia areata. Br J. Dermatol. 1996 December; 135(6):942-8.
¹²¹² Tobin D J, Hagen E, Botchkarev V A, Paus R. Do hair bulb melanocytes undergo apoptosis during hair follicle regression (catagen)? Invest Dermatol. 1998 December; 111(6):941-7.
¹²¹³ Ahmed A A, Nordlind K, Schultzberg M, Braketihoff J, Bristulf J, Novick D, Svenson S B, Azizi M, Liden S. Immunohistochemical studies of proinflammatory cytokines and their receptors in hair follicles of normal human skin. Acta Derm Venereol. 1996 September; 76(5):348-52.
¹²¹⁴ Deyerle K L, Sims J E, Dower S K, Bothwell M A. Pattern of IL-1 receptor gene expression suggests role in noninflammatory processes. J. Immunol. 1992 Sep. 1; 149(5):1657-65.
¹²¹⁵ Botchkarev V A, Botchkareva N V, Albers K M, Chen L H, Welker P, Paus R. A role for p75 neurotrophin receptor in the control of apoptosis-driven hair follicle regression. FASEB J. 2000 October; 14(13):1931-42.
¹²¹⁶ Botchkarev V A, Welker P, Albers K M, Botchkareva N V, Metz M, Lewin G R, Bulfone-Paus S, Peters E M, Lindner G, Paus R. A new role for neurotrophin-3: involvement in the regulation of hair follicle regression (catagen). Am J Pathol. 1998 September; 153(3):785-99.
¹²¹⁷ Foitzik K, Lindner G, Mueller-Roever S, Maurer M, Botchkareva N, Botchkarev V, Handjiski B. Metz M, Hibino T, Soma T, Dotto G P, Paus R. Control of murine hair follicle regression (catagen) by TGF-beta1 in vivo. FASEB J. 2000 April; 14(5):752-60.
¹²¹⁸ Welker P, Foitzik K, Bulfone-Paus S, Henz B M, Paus R. Hair cycle-dependent changes in the gene expression and protein content of transforming factor beta 1 and beta 3 in murine skin. Arch Dermatol Res. 1997 August; 289(9):554-7.
¹²¹⁹ Courtois M, Loussouarn G, Hourseau C, Grollier J F. Hair cycle and alopecia. Skin Pharmacol. 1994; 7(1-2):84-9.
¹²²⁰ Courtois M, Loussouam G, Hourseau C, Grollier J F. Ageing and hair cycles. Br J Dennatol. 1995 January; 132(1):86-93.
¹²²¹ Randall V A, Hibberts N A, Hamada K. A comparison of the culture and growth of dermal papilla cells from hair follicles from non-balding and balding (androgenetic alopecia) scalp. Br J. Dermatol. 1996 March; 134(3):437-44.
¹²²² Alcaraz M V, Villena A, Perez de Vargas I. Quantitative study of the human hair follicle in normal scalp and androgenetic alopecia. J Cutan Pathol. 1993 August; 20(4):344-9.
¹²²³ Whiting D A. Possible mechanisms of miniaturization during androgenetic alopecia or pattern hair loss. J Am Acad Dermatol. 2001 September; 45(3 Suppl):S81-6.
¹²²⁴ Chanda S, Robinette C L, Couse J F, Smart R C. 17beta-estradiol and ICI-182780 regulate the hair follicle cycle in mice through an estrogen receptor-alpha pathway. Am J Physiol Endocrinol Metab. 2000 February; 278(2):E202-10.
¹²²⁵ Guarrera M, Rebora A. Anagen hairs may fail to replace telogen hairs in early androgenic female alopecia. Dermatology. 1996; 192(1):28-31.
¹²²⁶ Oh H S, Smart R C. An estrogen receptor pathway regulates the telogen-anagen hair follicle transition and influences epidermal cell proliferation. Proc Natl Acad Sci USA. 1996 Oct. 29; 93(22): 12525-30.
¹²²⁷ Smart R C, Oh H S, Chanda S, Robinette C L. Effects of 17-beta-estradiol and ICI 182 780 on hair growth in various strains of mice. J Investig Dermatol Symp Proc. 1999 December; 4(3):285-9.
¹²²⁸ Sawaya M E, Honig L S, Hsia S L. Increased androgen binding capacity in sebaceous glands in scalp of male-pattern baldness. J Invest Dermatol. 1989 January; 92(1):91-5.
¹²²⁹ Hodgins M B, Choudhry R, Parker G, Oliver R F, Jahoda C A, Withers A P, Brinkmann A O, van der Kwast T H, Boersma W J, Lammers K M, Wong T K, Wawrzyniak C J, Warren R. Androgen receptors in dermal papilla cells of scalp hair follicles in male pattern baldness. Ann N Y Acad. Sci. 1991 Dec. 26; 642:448-51.
¹²³⁰ Hibberts N A, Howell A E, Randall V A. Balding hair follicle dermal papilla cells contain higher levels of androgen receptors than those from non-balding scalp. J Endocrinol. 1998 January; 156(1):59-65.
¹²³¹ Itami S, Kurata S, Sonoda T, Takayasu S. Interaction between dermal papilla cells and follicular epithelial cells in vitro: effect of androgen. Br J. Dermatol. 1995 April; 132(4):527-32.
¹²³² Elliott K, Stephenson T J, Messenger A G. Differences in hair follicle dermal papilla volume are due to extracellular matrix volume and cell number: implications for the control of hair follicle size and androgen responses. J Invest Dermatol. 1999 December; 113(6):873-7.
¹²³³ Harmon C S, Nevins T D, Bollag W B. Protein kinase C inhibits human hair follicle growth and hair fibre production in organ culture. Br J. Dermatol. 1995 November; 133(5):686-93.
¹²³⁴ Hoffmann R, Eicheler W, Wenzel E, Happle R. Interleukin-1beta-induced inhibition of hair growth in vitro is mediated by cyclic AMP. J Invest Dermatol. 1997 January; 108(1):40-2.
¹²³⁵ Kondo S, Hozumi Y, Aso K. Organ culture of human scalp hair follicles: effect of testosterone and oestrogen on hair growth. Arch Dermatol Res. 1990; 282(7):442-5.
¹²³⁶ Hoffmann R. The potential role of cytokines and T-cells in alopecia areata. J Investig Dermatol Symp Proc. 1999 December; 4(3):235-8. Review.
¹²³⁷ Groves R W, Mizutani H, Kieffer J D, Kupper T S. Inflammatory skin disease in transgenic mice that express high levels of interleukin 1 alpha in basal epidermis. Proc Natl Acad Sci USA. 1995 Dec. 5; 92(25):11874-8.
¹²³⁸ Xiong Y, Harmon C S. Interleukin-1 beta is differentially expressed by human dermal papilla cells in response to PKC activation and is a potent inhibitor of human hair follicle growth in organ culture. J Interferon Cytokine Res. 1997 March; 17(3):151-7.
¹²³⁹ Cotton S G, Nixon J M, Carpenter R G, Evans D W. Factors discriminating men with coronary heart disease from healthy controls. Br Heart J 1972 May; 34(5):458-64.
¹²⁴⁰ Lesko S M, Rosenberg L, Shapiro S. A case-control study of baldness in relation to myocardial infarction in men. JAMA. 1993 Feb. 24; 269(8):998-1003.
¹²⁴¹ Herrera C R, D'Agostino R B, Gerstman B B, Bosco L A, Belanger A J. Baldness and coronary heart disease rates in men from the Framingham Study. Am J Epidemiol. 1995 Oct. 15; 142(8):828-33.
¹²⁴² Lotufo P A, Chae C U, Ajani U A, Hennekens C H, Manson J E. Male pattern baldness and coronary heart disease: the Physicians' Health Study. Arch Intern Med. 2000 Jan. 24; 160(2):165-71.
¹²⁴³ Matilainen V A, Makinen P K, Keinanen-Kiukaanniemi S M. Early onset of androgenetic alopecia associated with early severe coronary heart disease: a population-based, case-control study. J Cardiovasc Risk. 2001 June; 8(3): 147-51.
¹²⁴⁴ Matilainen V, Koskela P, Keinanen-Kiukaanniemi S. Early androgenetic alopecia as a marker of insulin resistance. Lancet. 2000 Sep. 30; 356(9236):1165-6:
¹²⁴⁵ Piacquadio D J, Rad F S, Spellman M C, Hollenbach K A. Obesity and female androgenic alopecia: a cause and an effect? J Am Acad Dermatol. 1994 June; 30(6):1028-30.
¹²⁴⁶ Denmark-Wahnefried W, Schildkraut J M, Thompson D, Lesko S M, McIntyre L, Schwingl P, Paulson D F, Robertson C N, Anderson E E, Walther P J. Early onset baldness and prostate cancer risk. Cancer Epidemiol Biomarkers Prev. 2000 March; 9(3):325-8.
¹²⁴⁷ Hawk E, Breslow R A, Graubard B I. Male pattern baldness and clinical prostate cancer in the epidemiologic follow-up of the first National Health and Nutrition Examination Survey. Cancer Epidemiol Biomarkers Prev. 2000 May; 9(5):523-7.
¹²⁴⁸ Oh B R, Kim S J, Moon J D, Kim H N, Kwon D D, Won Y H, Ryu S B, Park Y I. Association of benign prostatic hyperplasia with male pattern baldness. Urology. 1998 May; 51(5):744-8.
¹²⁴⁹ Muthalif M M, Benter I F, Karzoun N, Fatima S, Harper J, Uddin M R, Malik K U. 20-Hydroxyeicosatetraenoic acid mediates calcium/calmodulin-dependent protein kinase II-induced mitogen-activated protein kinase activation in vascular smooth muscle cells. Proc Natl Acad Sci USA. 1998 Oct. 13; 95(21):12701-6.
¹²⁵⁰ Wen Y, Nadler J L, Gonzales N, Scott S, Clauser E, Natarajan R. Mechanisms of ANG II-induced mitogenic responses: role of 12-lipoxygenase and biphasic MAP kinase. Am J. Physiol. 1996 October; 271(4 Pt 1):C1212-20.
¹²⁵¹ Rao G N, Baas A S, Glasgow W C, Eling T E, Runge M S, Alexander R W. Activation of mitogen-activated protein kinases by arachidonic acid and its metabolites in vascular smooth muscle cells. J Biol. Chem. 1994 Dec. 23; 269(51):32586-91.
¹²⁵² Chen J K, Wang D W, Falck J R, Capdevila J, Harris R C. Transfection of an active cytochrome P450 arachidonic acid epoxygenase indicates that 14,15-epoxyeicosatrienoic acid functions as an intracellular second messenger in response to epidermal growth factor. J Biol. Chem. 1999 Feb. 19; 274(8):4764-9.
¹²⁵³ Bylund J, Ericsson J, Oliw E H. Analysis of cytochrome P450 metabolites of arachidonic and linoleic acids by liquid chromatography-mass spectrometry with ion trap MS. Anal Biochem. 1998 Dec. 1; 265(1):55-68.
¹²⁵⁴ Imaoka S, Wedlund P J, Ogawa H, Kimura S, Gonzalez F J, Kim H Y. Identification of CYP2C23 expressed in rat kidney as an arachidonic acid epoxygenase. J Pharmacol Exp Ther. 1993 November; 267(2): 1012-6.
¹²⁵⁵ Zeldin D C, Plitman J D, Kobayashi J, Miller R F, Snapper J R, Falck J R, Szarek J L, Philpot R M, Capdevila J H. The rabbit pulmonary cytochrome P450 arachidonic acid metabolic pathway: characterization and significance. J Clin Invest. 1995 May; 95(5):2150-60.
¹²⁵⁶ Rifkind A B, Lee C, Chang T K, Waxman D J. Arachidonic acid metabolism by human cytochrome P450s 2C8, 2C9, 2E1, and 1A2: regioselective oxygenation and evidence for a role for CYP2C enzymes in arachidonic acid epoxygenation in human liver microsomes. Arch Biochem Biophys. 1995 Jul. 10; 320(2):380-9.
¹²⁵⁷ Luo G, Zeldin D C, Blaisdell J A, Hodgson E, Goldstein J A. Cloning and expression of murine CYP2Cs and their ability to metabolize arachidonic acid. Arch Biochem Biophys. 1998 Sep. 1; 357(1):45-57.
¹²⁵⁸ Keeney D S, Skinner C, Travers J B, Capdevila J H, Nanney L B, King L E Jr, Waterman M R. Differentiating keratinocytes express a novel cytochrome P450 enzyme, CYP2B19, having arachidonate monooxygenase activity. J Biol. Chem. 1998 Nov. 27; 273(48):32071-9.
¹²⁵⁹ Kidd R S, Straughn A B, Meyer M C, Blaisdell J, Goldstein J A, Dalton J T. Pharmacokinetics of chlorpheniramine, phenyloin, glipizide and nifedipine in an individual homozygous for the CYP2C9*3 allele. Pharmacogenetics. 1999 February; 9(1):71-80.
¹²⁶⁰ Ring B J, Binkley S N, Vandenbranden M, Wrighton S A. In vitro interaction of the antipsychotic agent olanzapine with human cytochromes P450 CYP2C9, CYP2C19, CYP2D6 and CYP3A. Br J Clin Pharmacol. 1996 March; 41(3):181-6.
¹²⁶¹ Miners J O, Birkett D J. Cytochrome P4502C9: an enzyme of major importance in human drug metabolism. Br J Clin Pharmacol. 1998 June; 45(6):525-38. Review.
¹²⁶² Egger J, Brett E M. Effects of sodium valproate in 100 children with special reference to weight. Br Med J (Clin Res Ed). 1981 Aug. 29; 283(6291):577-81.
¹²⁶³ Campbell I W, Menzies D G, Chalmers J, McBain A M, Brown I R. One year comparative trial of metformin and glipizide in type 2 diabetes mellitus. Diabete Metab 1994 July-August; 20(4):394-400.
¹²⁶⁴ Petersen K U. Review article: omeprazole and the cytochrome P450 system. Aliment Pharmacol Ther 1995 February; 9(1): 1-9.
¹²⁶⁵ Meyer U A. Interaction of proton pump inhibitors with cytochromes P450: consequences for drug interactions. Yale J Biol Med 1996 May-June; 69(3):203-9.
¹²⁶⁶ Hogan R E, Bertrand M E, Deaton R L, Sommerville K W. Total percentage body weight changes during add-on therapy with tiagabine, carbamazepine and phenyloin. Epilepsy Res. 2000 August; 41(1):23-8.
¹²⁶⁷ Mattson R H, Cramer J A, Collins J F. A comparison of valproate with carbamazepine for the treatment of complex partial seizures and secondarily generalized tonic-clonic seizures in adults. The Department of Veterans Affairs Epilepsy Cooperative Study No. 264, Group. N Engl J. Med. 1992 Sep. 10; 327(11):765-71.
¹²⁶⁸ Sadeque A J M, Fisher M B, Korzekwa K R, Gonzalez F J, Rettie A E. Human CYP2C9 and CYP2A6 mediate formation of the hepatotoxin 4-ene-valproic acid. J Pharmacol Exp Ther. 1997 November; 283(2):698-703.
¹²⁶⁹ Bruni J, Wilder B J. Valproic acid. Review of a new antiepileptic drug. Arch Neurol 1979 July; 36(7):393 8.
¹²⁷⁰ Zaccara G, Campostrini R. Paganini M, Messori A, Valenza T, Arnetoli G, Zappoli R. Long-term treatment with sodium valproate: monitoring of venous ammonia concentrations and adverse effects. Ther Drug Monit. 1987; 9(1):34-40.
¹²⁷¹ Sharpe C, Buchanan N. Juvenile myoclonic epilepsy: diagnosis, management and outcome. Med J Aust. 1995 Feb. 6; 162(3):133-4.
¹²⁷² Song J C, White C M. Pharmacologic, pharmacokinetic, and therapeutic differences among angiotensin 11 receptor antagonists. Pharmacotherapy. 2000 February; 20(2):130-9. Review
¹²⁷³ Meadowcroft A M, Williamson K M, Patterson J H, Hinderliter A L, Pieper J A. The effects of fluvastatin, a CYP2C9 inhibitor, on losartan pharmacokinetics in healthy volunteers. J Clin Pharmacol. 1999 April; 39(4):418-24.
¹²⁷⁴ Camargo M J, von Lutterotti N, Pecker M S, James G D, Timmermans P B, Laragh J H. DuP 753 increases survival in spontaneously hypertensive stroke-prone rats fed a high sodium diet. Am J Hypertens. 1991 April; 4(4 Pt 2):341S-345S.
¹²⁷⁵ Transon C, Leemann T, Dayer P. In vitro comparative inhibition profiles of major human drug metabolising cytochrome P450 isozymes (CYP2C9, CYP2D6 and CYP3A4) by HMG-CoA reductase inhibitors. Eur J Clin Pharmacol. 1996; 50(3):209-15.
¹²⁷⁶ Matthews P G, Wahlqvist M L, Marks S J, Myers K A, Hodgson J M. Improvement in arterial stiffness during hypolipidaemic therapy is offset by weight gain. Int J Obes Relat Metab Disord. 1993 October; 17(10):579-83.
¹²⁷⁷ Osser D N, Najarian D M, Dufresne R L. Olanzapine increases weight and serum triglyceride levels. J Clin Psychiatry. 1999 November; 60(11):767-70.
¹²⁷⁸ Koran L M, Ringold A L, Elliott M A. Olanzapine augmentation for treatment-resistant obsessive-compulsive disorder. J Clin Psychiatry. 2000 July; 61(7):514-7.
¹²⁷⁹ Fang J, Coutts R T, McKenna K F, Baker G B. Elucidation of individual cytochrome P450 enzymes involved in the metabolism of clozapine. Naunyn Schmiedebergs Arch Pharmacol. 1998 November; 358(5):592-9.
¹²⁸⁰ Prior T I, Chue P S, Tibbo P, Baker G B. Drug metabolism and atypical antipsychotics. Eur Neuropsychopharmacol. 1999 June; 9(4):301-9. Review.
¹²⁸¹ Olesen O V, Linnet K. Fluvoxamine-Clozapine drug interaction: inhibition in vitro of five cytochrome P450 isoforms involved in clozapine metabolism. J Clin Psychopharmacol. 2000 February; 20(1): 35-42.
¹²⁸² Schmider J, Greenblatt D J, von Moltke L L, Karsov D, Shader R I. Inhibition of CYP2C9 by selective serotonin reuptake inhibitors in vitro: studies of phenyloin p-hydroxylation. Br J Clin Pharmacol. 1997 November; 44(5):495-8.
¹²⁸³ Harvey B H, Bouwer C D. Neuropharmacology of paradoxic weight gain with selective serotonin reuptake inhibitors. Clin Neuropharmacol. 2000 March-April; 23(2):90-7. Review.
¹²⁸⁴ Sansone R A, Wiederman M W, Shrader J A. Naturalistic study of the weight effects of amitriptyline, fluoxetine, and sertraline in an outpatient medical setting. J Clin Psychopharmacol. 2000 April; 20(2):272-4.
¹²⁸⁵ Michelson D, Amsterdam J D, Quitkin F M, Reimherr F W, Rosenbaum J F, Zajecka J, Sundell K L, Kim Y, Beasley C M Jr. Changes in weight during a 1-year trial of fluoxetine. Am J Psychiatry. 1999 August; 156(8):1170-6.
¹²⁸⁶ Darga L L, Carroll-Michals L, Botsford S J, Lucas C P. Fluoxetine's effect on weight loss in obese subjects. Am J Clin Nutr. 1991 August; 54(2):321-5.
¹²⁸⁷ Lasker J M, Wester M R, Aramsombatdee E, Raucy J L. Characterization of CYP2CI9 and CYP2C9 from human liver: respective roles in microsomal tolbutamide, S-mephenyloin, and omeprazole hydroxylations. Arch Biochem Biophys. 1998 May 1; 353(1):16-28.
¹²⁸⁸ Wissler R W, Borensztajn J, Rubenstein A, Getz G, Vesselinovitch D The effects of tolbutamide on the development of atherosclerosis in rhesus monkeys fed an average American table-prepared diet. Adv Exp Med Biol. 1975; 63:379-80.
¹²⁸⁹ Ballagi-Pordany G, Koltai M Z, Aranyi Z, Pogatsa G. Direct effect of hypoglycemic sulphonylureas on the cardiovascular system of dogs. Diabetes Res Clin Pract. 1991 January; 11(1):47-52.
¹²⁹⁰ Grimm S W, Dyroff M C. Inhibition of human drug metabolizing cytochromes P450 by anastrozole, a potent and selective inhibitor of aromatase. Drug Metab Dispos. 1997 May; 25(5):598-602.
¹²⁹¹ Wiseman L R, Adkins J C. Anastrozole. A review, of its use in the management of postmenopausal women with advanced breast cancer. Drugs Aging. 1998 October; 13(4):321-32. Review.
¹²⁹² Lonning P E. Aromatase inhibitors and their future role in post-menopausal women with early breast cancer. Br J Cancer. 1998 September; 78 Suppl 4:12-5.
¹²⁹³ Buzdar A U. Anastrozole: a new addition to the armamentarium against advanced breast cancer. Am J Clin Oncol. 1998 April; 21(2):161-6. Review.
¹²⁹⁴ Jonat W. Clinical overview of anastrozole—a new selective oral aromatase inhibitor. Oncology. 1997; 54 Suppl 2:15-8. Review.
¹²⁹⁵ Buzdar A U, Jonat W, Howell A, Plourde P V. ARIMIDEX: a potent and selective aromatase inhibitor for the treatment of advanced breast cancer. J Steroid Biochem Mol. Biol. 1997 April; 61(3-6):145-9. Review.
¹²⁹⁶ Hannaford M. Role of new selective aromatase inhibitor in therapy for metastatic breast cancer in postmenopausal women. Nurse Pract. 1997 March; 22(3): 195-6, 201-2.
¹²⁹⁷ Buzdar A U, Jones S E, Vogel C L, Wolter J, Plourde P, Webster A. A phase III trial comparing anastrozole (1 and 10 milligrams), a potent and selective aromatase inhibitor, with megestrol acetate in postmenopausal women with advanced breast carcinoma. Arimidex Study Group. Cancer. 1997 Feb. 15; 79(4):730-9.
¹²⁹⁸ Buzdar A, Jonat W, Howell A, Jones S E, Blomqvist C, Vogel C L, Eiermann W, Wolter J M, Azab M, Webster A, Plourde P V. Anastrozole, a potent and selective aromatase inhibitor, versus megestrol acetate in postmenopausal women with advanced breast cancer: results of overview analysis of two phase III trials. Arimidex Study Group. J Clin Oncol. 1996 July; 14(7):2000-11.
¹²⁹⁹ Jonat W, Howell A, Blomqvist C, Eiermann W, Winblad G, Tyrrell C, Mauriac L, Roche H, Lundgren S, Hellmund R, Azab M. A randomised trial comparing two doses of the new selective aromatase inhibitor anastrozole (Arimidex) with megestrol acetate in postmenopausal patients with advanced breast cancer. Eur J Cancer. 1996 March; 32A(3):404-12.
¹³⁰⁰ Khaliq Y, Gallicano K, Seguin I, Fyke K, Carignan G, Bulman D, Badley A, Cameron D W. Single and multiple dose pharmacokinetics of nelfinavir and CYP2C19 activity in human immunodeficiency virus-infected patients with chronic liver disease. Br J Clin Pharmacol. 2000 August; 50(2):108-15.
¹³⁰¹ Lillibridge J H, Liang B H, Kerr B M, Webber S, Quart B, Shetty B V, Lee C A. Characterization of the selectivity and mechanism of human cytochrome P450 inhibition by the human immunodeficiency virus-protease inhibitor nelfinavir mesylate. Drug Metab Dispos. 1998 July; 26(7):609-16.
¹³⁰² Muirhead G J, Wulff M B, Fielding A, Kleinermans D, Buss N. Pharmacokinetic interactions between sildenafil and saquinavir/ritonavir. Br J Clin Pharmacol. 2000 August; 50(2):99-107.
¹³⁰³ Kumar G N, Dykstra J, Roberts E M, Jayanti V K, Hickman D, Uchic J, Yao Y, Surber B, Thomas S, Granneman GRPotent inhibition of the cytochrome P-450 3A-mediated human liver microsomal metabolism of a novel HIV protease inhibitor by ritonavir: A positive drug-drug interaction. Drug Metab Dispos. 1999 August; 27(8):902-8.
¹³⁰⁴ Kumar G N, Rodrigues A D, Buko A M, Denissen J F. Cytochrome P450-mediated metabolism of the HIV-1 protease inhibitor ritonavir (ABT-538) in human liver microsomes. J Pharmacol Exp Ther. 1996 April; 277(1):423-31.
¹³⁰⁵ Eagling V A, Back D J, Barry M G. Differential inhibition of cytochrome P450 isoforms by the protease inhibitors, ritonavir, saquinavir and indinavir. Br J Clin Pharmacol. 1997 August; 44(2):190-4.
¹³⁰⁶ Fung H B, Kirschenbaum H L, Hameed R. Amprenavir: a new human immunodeficiency virus type I protease inhibitor. Clin Ther. 2000 May; 22(5):549-72. Review.
¹³⁰⁷ Fisslthaler B, Hinsch N, Chataigneau T, Popp R, Kiss L, Busse R, Fleming I. Nifedipine increases cytochrome P4502C expression and endothelium-derived hyperpolarizing factor-mediated responses in coronary arteries. Hypertension. 2000 August; 36(2):270-5.
¹³⁰⁸ Krakoff L R. Effectiveness of nifedipine gastrointestinal therapeutic system for treatment of hypertension: results of the MATH Trial. J Cardiovasc Pharmacol. 1993; 21 Suppl 2:S14-7.
¹³⁰⁹ Maccario M, Oleandri S E, Avogadri E, Rossetto R, Grottoli S, Procopio M, Camanni F, Ghigo E. Effects of 3-month nifedipine treatment on endocrine-metabolic parameters in patients with abdominal obesity and mild hypertension. J Endocrinol Invest. 1998 January; 21(1):56-63.
¹³¹⁰ Andronico G, Piazza G, Mangano M T, Mule G, Carone M B, Cerasola G. Nifedipine vs. enalapril in treatment of hypertensive patients with glucose intolerance. J Cardiovasc Pharmacol. 1991; 18 Suppl 10:S52-4.
¹³¹¹ Barreiro P, Soriano V, Blanco F, Casimiro C, de la Cruz J J, Gonzalez-Lahoz J. Risks and benefits of replacing protease inhibitors by nevirapine in HIV-infected subjects under long-term successful triple combination therapy. AIDS. 2000 May 5; 14(7):807-12.
¹³¹² Mulligan K, Grunfeld C, Tai V W, Algren H, Pang M, Chernoff D N, Lo J C, Schambelan M. Hyperlipidemia and insulin resistance are induced by protease inhibitors independent of changes in body composition in patients with HIV infection. J Acquir Immune Defic Syndr. 2000 Jan. 1; 23(1):35-43.
¹³¹³ Gervasoni C, Ridolfo A L, Trifiro G, Santambrogio S, Norbiato G, Musicco M, Clerici M, Galli M, Moroni M. Redistribution of body fat in HIV-infected women undergoing combined antiretroviral therapy. AIDS. 1999 Mar. 11; 13(4):465-71.
¹³¹⁴ Carr A, Copper D A. A randomized, multicenter study of protease inhibitor (PI) substitution in aviremic patients with antiretroviral (ARV) lipodystrophy syndrome. 7th Conference on Retroviruses and Opportunistic Infections. 2000 (Abstract).
¹³¹⁵ Martinez E, Blanco J L, Garcia M A, Buira E, Bianchi L, Conget I, Casamitjana R, Gatell J M. Impact of switching from HIV-1 protease Inhibitors (PI) to efavirenz (EFV) in patients with lipodystrophy. 7th Conference on Retroviruses and Opportunistic Infections. 2000 (Abstract).
¹³¹⁶ Passalaris J D, Sepkowitz K A, Glesby M J. Coronary artery disease and human immunodeficiency virus infection. Clin Infect Dis. 2000 September; 31(3):787-97.
¹³¹⁷ Andrews G K, Adamson E D. Butyrate selectively activates the metallothionein gene in teratocarcinoma cells and induces hypersensitivity to metal induction. Nuclein Acids Research. 1987 15(13): 5461-5475.
¹³¹⁸ Thomas D J, Angle C R, Swanson S A, Caffrey T C. Effect of sodium butyrate on metallothionein induction and cadmium cytotoxicity in ROS 17/2.8 cells. Toxicology 1991 Feb. 11; 66(1):35-46.
¹³¹⁹ Liu J, McKim J M Jr, Liu Y P, Klaassen C D. Effects of butyrate homologues on metallothionein induction in rat primary hepatocyte cultures. In Vitro Cell Dev Biol 1992 May; 28A(5):320-6.
¹³²⁰ Ludwig D S, Pereira M A, Kroenke C H, Hilner J E, Van Horn L, Slattery M L, Jacobs D R Jr. Dietary fiber, weight gain, and cardiovascular disease risk factors in young adults. JAMA. 1999 Oct. 27; 282(16): 1539-46.
¹³²¹ Rigaud D, Ryttig K R, Angel L A, Apfelbaum M. Overweight treated with energy restriction and a dietary fibre supplement: a 6-month randomized, double-blind, placebo-controlled trial. Int J Obes. 1990 September; 14(9):763-9.
¹³²² Ryttig K R, Telines G, Haegh L, Boe E, Fagerthun H. A dietary fibre supplement and weight maintenance after weight reduction: a randomized, double-blind, placebo-controlled long-term trial. Int J Obes. 1989; 13(2):165-71.
¹³²³ Piliang W G, Djojosoebagio S, Suprayogi A. Soybean hull and its effect on atherosclerosis in non-human primates (Macaca fascicularis). Biomed Environ Sci 1996 September; 9(2-3):137-43.
¹³²⁴ Kim Y I. AGA technical review: impact of dietary fiber on colon cancer occurrence. Gastroenterology. 2000 June; 118(6): 1235-57.
¹³²⁵ Madar Z, Stark A. Dietary fiber and colorectal cancer. N Engl J. Med. 1999 Jun. 17; 340(24): 1925-6; discussion 1926.
¹³²⁶ Camire M E. Dietary fiber and colorectal cancer. N Engl J. Med. 1999 Jun. 17; 340(24):1926.
¹³²⁷ Mohandas K M. Dietary fiber and colorectal cancer. N Engl J. Med. 1999 Jun. 17; 340(24):1925; discussion 1926.
¹³²⁸ Heaton K W, Lewis S J. Dietary fiber and colorectal cancer. N Engl J. Med. 1999 Jun. 17; 340(24):1925; discussion 1926.
¹³²⁹ Cummings J H, Southgate D A. Dietary fiber and colorectal cancer. N Engl J. Med. 1999 Jun. 17; 340(24):1925; discussion 1926.
¹³³⁰ Ravin N D. Dietary fiber and colorectal cancer. N Engl J. Med. 1999 Jun. 17; 340(24):1924-5; discussion 1926.
¹³³¹ Reddy B S. Role of dietary fiber in colon cancer: an overview. Am J. Med. 1999 Jan. 25; 106(1A): 16s⁻¹⁹S; discussion 50S-51 S.
¹³³² Reddy B S. Prevention of colon carcinogenesis by components of dietary fiber. Anticancer Res. 1999 September-October; 19(5A):3681-3.
¹³³³ Earnest D L, Einspahr J G, Alberts D S. Protective role of wheat bran fiber: data from marker trials. Am J. Med. 1999 Jan. 25; 106(1A):32S-37S.
¹³³⁴ Kritchevsky D. Protective role of wheat bran fiber: preclinical data. Am J. Med. 1999 Jan. 25; 106(1A):28S-31S.
¹³³⁵ Cohen L A. Dietary fiber and breast cancer. Anticancer Res. 1999 September-October; 19(5A):3685-8.
¹³³⁶ Wolin M J, Miller T L, Yerry S, Zhang Y, Bank S, Weaver G A. Changes of fermentation pathways of fecal microbial communities associated with a drug treatment that increases dietary starch in the human colon. Appl Environ Microbiol 1999 July; 65(7):2807-12.
¹³³⁷ Wolever T M, Chiasson J L, Josse R G, Hunt J A, Palmason C, Rodger N W, Ross S A, Ryan E A, Tan M H. Small weight loss on long-term acarbose therapy with no change in dietary pattern or nutrient intake of individuals with non-insulin-dependent diabetes. Int J Obes Relat Metab Disord. 1997 September; 21(9):756-63.
¹³³⁸ Huyer G, Liu S, Kelly J, Moffat J, Payette P, Kennedy B, Tsaprailis G, Gresser M J, Ramachandran C. Mechanism of inhibition of protein-tyrosine phosphatases by vanadate and pervanadate. J Biol Chem 1997 Jan. 10; 272(2):843-51.
¹³³⁹ Wang Y Z, Bonner J C. Mechanism of extracellular signal-regulated kinase (ERK)-1 and ERK-2 activation by vanadium pentoxide in rat pulmonary myofibroblasts. Am J Respir Cell Mol. Biol. 2000 May; 22(5):590-6.
¹³⁴⁰ Zhao Z, Tan Z, Diltz C D, You M, Fischer E H. Activation of mitogen-activated protein (MAP) kinase pathway by pervanadate, a potent inhibitor of tyrosine phosphatases. J Biol. Chem. 1996 Sep. 6; 271(36):22251-5.
¹³⁴¹ Pandey S K, Chiasson J L, Srivastava A K. Vanadium salts stimulate mitogen-activated protein (MAP) kinases and ribosomal S6 kinases. Mol Cell Biochem. 1995 Dec. 6-20; 153(1-2):69-78.
¹³⁴² D'Onofrio F, Le M Q, Chiasson J L, Srivastava A K. Activation of mitogen activated protein (MAP) kinases by vanadate is independent of insulin receptor autophosphorylation. FEBS Lett. 1994 Mar. 7; 340(3):269-75.
¹³⁴³ Darville M I, Antoine I V, Rousseau G G. Characterization of an enhancer upstream from the muscle-type promoter of a gene encoding 6-phosphofructo-2-kinase/fructose-2,6-bisphosphatase. Nucleic Acids Res. 1992 Jul. 25; 20(14):3575-83.
¹³⁴⁴ Dupriez V J, Darville M I, Antoine I V, Gegonne A, Ghysdael J, Rousseau G G. Characterization of a hepatoma mRNA transcribed from a third promoter of a 6-phosphofructo-2-kinase/fructose-2,6-bisphosphatase-encoding gene and controlled by ets oncogene-related products. Proc Natl Acad Sci USA. 1993 Sep. 1; 90(17):8224-8.
¹³⁴⁵ Miralpeix M, Carballo E, Bartrons R, Crepin K, Hue L, Rousseau G G. Oral administration of vanadate to diabetic rats restores liver 6-phosphofructo-2-kinase content and mRNA. Diabetologia. 1992 March; 35(3):243-8.
¹³⁴⁶ Inoue H, Kaku K, Matsutani A, Tao T, Ayame H, Kaneko T. Insulin-like effects of vanadate on rat liver 6-phosphofructo-2-kinase/fructose-2,6-bisphosphatase mRNA and protein inductions in diabetic rats. Endocr J. 1994 February; 41(1):75-82.
¹³⁴⁷ Herrman C E, Sanders R A, Klaunig J E, Schwarz L R, Watkins J B 3rd. Decreased apoptosis as a mechanism for hepatomegaly in streptozotocin-induced diabetic rats. Toxicol Sci 1999 July; 50(1): 146-51.
¹³⁴⁸ Pugazhenthi S, Tanha F, Dahl B, Khandelwal R L. Decrease in protein tyrosine phosphatase activities in vanadate-treated obese Zucker (fa/fa) rat liver. Mol Cell Biochem. 1995 Dec. 6-20; 153(1-2): 125-9.
¹³⁴⁹ McNeill J H, Orvig C. Bis(maltolato)oxovanadium compositions for the treatment of elevated blood sugar. U.S. Pat. No. 5,527,790, Jun. 18, 1996.
¹³⁵⁰ Dai S, Thompson K H, McNeill J H. One-year treatment of streptozotocin-induced diabetic rats with vanadyl sulphate. Pharmacol Toxicol 1994 February; 74(2):101-9.
¹³⁵¹ Bhanot S, McNeill J H. Vanadyl sulfate lowers plasma insulin and blood pressure in spontaneously hypertensive rats. Hypertension. 1994 November; 24(5):625-32.
¹³⁵² Cruz T F, Morgan A, Min W. In vitro and in vivo antineoplastic effects of orthovanadate. Mol Cell Biochem. 1995 Dec. 6-20; 153(1-2):161-6.
¹³⁵³ Bishayee A, Chatterjee M. Inhibitory effect of vanadium on rat liver carcinogenesis initiated with diethylnitrosamine and promoted by phenobarbital. Br J Cancer. 1995 June; 71(6):1214-20.
¹³⁵⁴ Liasko R, Kabanos T A, Karkabounas S, Malamas M, Tasiopoulos A J, Stefanou D, Collery P, Evangelou A. Beneficial effects of a vanadium complex with cysteine, administered at low doses on benzo(alpha)pyrene-induced leiomyosarcomas in Wistar rats. Anticancer Res. 1998 September-October; 18(5A):3609-13.
¹³⁵⁵ Goldfine A B, Simonson D C, Folli F, Patti M E, Kahn C R. In vivo and in vitro studies of vanadate in human and rodent diabetes mellitus. Mol Cell Biochem. 1995 Dec. 6-20; 153(1-2):217-31.
¹³⁵⁶ Brichard S M, Henquin J C. The role of vanadium in the management of diabetes. Trends Pharmacol Sci. 1995 August; 16(8):265-70.
¹³⁵⁷ Elchebly M, Payette P, Michaliszyn E, Cromlish W, Collins S, Loy A L, Normandin D, Cheng A, Himms-Hagen J, Chan C C, kamachandran C, Gresser M J, Tremblay M L, Kennedy B P. Increased insulin sensitivity and obesity resistance in mice lacking the protein tyrosine phosphatase 1B gene. Science. 1999. Mar. 5; 283(5407): 1544-8;
¹³⁵⁸ Klaman L D, Boss O, Peroni O D, Kim J K, Martino J L, Zabolotny J M, Moghal N, Lubkin M, Kim Y B, Sharpe A H, Stricker-Krongrad A, Shulman G I, Neel B G, Kahn B B. Increased energy expenditure, decreased adiposity, and tissue-specific insulin sensitivity in protein-tyrosine phosphatase 1B-deficient mice. Mol Cell Biol. 2000 August; 20(15):5479-89.
¹³⁵⁹ Ojuka E O, Jones T E, Han D H, Chen M, Holloszy J O. Raising Ca2+ in L6 myotubes mimics effects of exercise on mitochondrial biogenesis in muscle. FASEB J. 2003 April; 17(6):675-81.
¹³⁶⁰ Baar K, Wende A R, Jones T E, Marison M, Nolte L A, Chen M, Kelly D P, Holloszy J O. Adaptations of skeletal muscle to exercise: rapid increase in the transcriptional coactivator PGC-1. FASEB J. 2002 December; 16(14):1879-86.
¹³⁶¹ Prasad K, Laxdal V A, Yu M, Raney B L. Evaluation of hydroxyl radical-scavenging property of garlic. Mol Cell Biochem. 1996 Jan. 12; 154(1):55-63.
¹³⁶² Ide N, Lau B H. Aged garlic extract attenuates intracellular oxidative stress. Phytomedicine 1999 May; 6(2): 125-31.
¹³⁶³ Efendy J L, Simmons D L, Campbell G R, Campbell J H. The effect of the aged garlic extract, ‘Kyolic’, on the development of experimental atherosclerosis. Atherosclerosis. 1997 Jul. 11; 132(1):37-42.
¹³⁶⁴ Jain R C, Konar DBEffect of garlic oil in experimental cholesterol atherosclerosis. Atherosclerosis. 1978 February; 29(2): 125-9.
¹³⁶⁵ Jain R C. Onion and garlic in experimental cholesterol induced atherosclerosis. Indian J Med Res. 1976 October; 64(10): 1509-15.
¹³⁶⁶ Bordia A, Arora S K, Kothari L K, Jain K C, Rathore B S, Rathore A S, Dube M K, Bhu N. The protective action of essential oils of onion and garlic in cholesterol-fed rabbits. Atherosclerosis. 1975 July-August; 22(1):103-9.
¹³⁶⁷ Breithaupt-Grogler K, Ling M, Boudoulas H, Belz G G. Protective effect of chronic garlic intake on elastic properties of aorta in the elderly. Circulation. 1997 Oct. 21; 96(8):2649-55.
¹³⁶⁸ Farrar D J, Bond M G, Riley W A, Sawyer J K. Anatomic correlates of aortic pulse wave velocity and carotid artery elasticity during atherosclerosis progression and regression in monkeys. Circulation. 1991 May; 83(5):1754-63.
¹³⁶⁹ Ali M, Thomson M, Afzal M. Garlic and onions: their effect on eicosanoid metabolism and its clinical relevance. Prostaglandins Leukot Essent Fatty Acids. 2000 February; 62(2):55-73.
¹³⁷⁰ Fleischauer A T, Poole C, Arab L. Garlic consumption and cancer prevention: meta-analyses of colorectal and stomach cancers. Am J Clin Nutr. 2000 October; 72(4):1047-52.
¹³⁷¹ Singh A, Shukla Y. Antitumour activity of diallyl sulfide on polycyclic aromatic hydrocarbon-induced mouse skin carcinogenesis. Cancer Lett. 1998 Sep. 25; 131(2):209-14.
¹³⁷² Singh S V, Mohan R R, Agarwal R, Benson P J, Hu X, Rudy M A, Xia H, Katoh A, Srivastava S K, Mukhtar H, Gupta V, Zaren H A. Novel anti-carcinogenic activity of an organosulfide from garlic: inhibition of H-RAS oncogene transformed tumor growth in vivo by diallyl disulfide is associated with inhibition of p211 Hr processing. Biochem Biophys Res Commun. 1996 Aug. 14; 225(2):660-5.
¹³⁷³ Spector S A. Oral ganciclovir. Adv Exp Med Biol 1999; 458:121-7.
¹³⁷⁴ Leflore S, Anderson P L, Fletcher C V. A risk-benefit evaluation of aciclovir for the treatment and prophylaxis of herpes simplex virus infections. Drug Saf 2000 August; 23(2):131-42.
¹³⁷⁵ Kesson A M. Use of aciclovir in herpes simplex virus infections. J Paediatr Child Health. 1998 February; 34(1); 9-13.
¹³⁷⁶ Ormrod D, Scott L J, Perry C M. Valaciclovir: a review of its long term utility in the management of genital herpes simplex virus and cytomegalovirus infections. Drugs 2000 April; 59(4):839-63.
¹³⁷⁷ Bell A R. Valaciclovir update. Adv. Exp Med Biol. 1999; 458:149-57.
¹³⁷⁸ Sacks S L, Wilson B. Famciclovir/penciclovir. Adv Exp Med Biol. 1999; 458:135-47.
¹³⁷⁹ Reddehase M J, Balthesen M, Rapp M, Jonjic S, Pavic I, Koszinowski U H. The conditions of primary infection define the load of latent viral genome in organs and the risk of recurrent cytomegalovirus disease. J Exp Med 1994 Jan. 1; 179(1):185-93.
¹³⁸⁰ Collins T, Pomeroy C, Jordan M C. Detection of latent cytomegalovirus DNA in diverse organs of mice. J Infect Dis 1993 September; 168(3):725-9.
¹³⁸¹ Steffens H P, Kurz S, Holtappels R, Reddehase M J. Preemptive CD8 T-cell immunotherapy of acute cytomegalovirus infection prevents lethal disease, limits the burden of latent viral genomes, and decreases the risk of virus recurrence. J. Virol. 1998 March; 72(3):1797-804.
¹³⁸² Thackray A M, Field H J. The effects of antiviral therapy on the distribution of herpes simplex virus type I to ganglionic neurons and its consequences during, immediately following and several months after treatment. J Gen Virol 2000 October; 81 Pt 10:2385-96.
¹³⁸³ Thackray A M, Field H J. Further evidence from a murine infection model that famciclovir interferes with the establishment of HSV-1 latent infections. J Antimicrob Chemother. 2000 June; 45(6):825-33.
¹³⁸⁴ Thackray A M, Field H J. Effects of famciclovir and valacyclovir on herpes simplex virus type I infection, latency, and reactivation in mice: how dissimilar are study results? J Infect Dis 2000 April; 181(4): 1517-8.
¹³⁸⁵ Field H J, Thackray A M. Early therapy with valaciclovir or famciclovir decreases but does not abrogate herpes simplex virus neuronal latency. Nucleosides Nucleotides Nucleic Acids. 2000 January-February; 19(1-2):461-70.
¹³⁸⁶ Thackray A M, Field H J. Famciclovir and valaciclovir differ in the prevention of herpes simplex virus type I latency in mice: a quantitative study. Antimicrob Agents Chemother 1998 July; 42(7):1555-62.
¹³⁸⁷ LeBlanc R A, Pesnicak L, Godleski M, Straus S E. Treatment of HSV-1 infection with immunoglobulin or acyclovir: comparison of their effects on viral spread, latency, and reactivation. Virology. 1999 Sep. 15; 262(1):230-6.
¹³⁸⁸ Bowden R A. Cytomegalovirus infections in transplant patients: methods of prevention of primary cytomegalovirus. Transplant Proc 1991 June; 23(3 Suppl 3):136-8, discussion 138.
¹³⁸⁹ Chou S W, Norman D J. The influence of donor factors other than serologic status on transmission of cytomegalovirus to transplant recipients. Transplantation 1988 July; 46(1):89-93.
¹³⁹⁰ Chou S W. Cytomegalovirus infection and reinfection transmitted by heart transplantation. J Infect Dis. 1987 May; 155(5):1054-6.
¹³⁹¹ Chou S W. Acquisition of donor strains of cytomegalovirus by renal-transplant recipients. N Engl J. Med. 1986 May 29; 314(22):1418-23.
¹³⁹² Grundy J E, Lui S F, Super M, Berry N J, Sweny P, Fernando O N, Moorhead J, Griffiths P D. Symptomatic cytomegalovirus infection in seropositive kidney recipients: reinfection with donor virus rather than reactivation of recipient virus. Lancet 1988 Jul. 16; 2(8603):132-5.
¹³⁹³ Grundy J E, Super M, Lui S, Sweny P, Griffiths P D. The source of cytomegalovirus infection in seropositive renal allograft recipients is frequently the donor kidney. Transplant Proc. 1987 February; 19(1 Pt 3):2126-8.
¹³⁹⁴ Grundy J E, Super M, Lui S F, Griffiths P D. Donor strains of cytomegalovirus in renal-transplant recipients. N Engl J. Med. 1986 Nov. 6; 315(19):1229.
¹³⁹⁵ Valantine H A, Gao S Z, Menon S G, Renlund D G, Hunt S A, Oyer P, Stinson E B, Brown B W Jr, Merigan T C, Schroeder J S. Impact of prophylactic immediate posttransplant ganciclovir on development of transplant atherosclerosis: a post hoc analysis of a randomized, placebo-controlled study. Circulation. 1999 Jul. 6; 100(1):61-6.
¹³⁹⁶ Sia I G, Patel R. New strategies for prevention and therapy of cytomegalovirus infection and disease in solid-organ transplant recipients. Clin Microbiol Rev. 2000January; 13(1):83-121.
¹³⁹⁷ Perry C M, Noble S. Didanosine: an updated review of its use in HIV infection. Drugs. 1999 December; 58(6):1099-135.
¹³⁹⁸ Bruisten S M, Reiss P, Loeliger A E, van Swieten P, Schuurman R, Boucher C A, Weverling G J, Huisman J G. Cellular proviral HIV type I DNA load persists after long-term RT-inhibitor therapy in HIV type I infected persons. AIDS Res Hum Retroviruses. 1998 Aug. 10; 14(12):1053-8.
¹³⁹⁹ Magnani M, Rossi L, Fraternale A, Casablanca A, Brandi G, Benatti U, De Flora A. Targeting antiviral nucleotide analogues to macrophages. J Leukoc Biol 1997 July; 62(1): 133-7.
¹⁴⁰⁰ Pauza C D, Trivedi P, McKechnie T S, Richman D D, Graziano F M. 2-LTR circular viral DNA as a marker for human immunodeficiency virus type I infection in vivo. Virology. 1994 December; 205(2):470-8.
¹⁴⁰¹ Chun T W, Stuyver L, Mizell S B, Ehler L A, Mican J A, Baseler M, Lloyd A L, Nowak M A, Fauci A S. Presence of an inducible HIV-1 latent reservoir during highly active antiretroviral therapy. Proc Natl Acad Sci USA. 1997 Nov. 25; 94(24):13193-7.
¹⁴⁰² Guo N L, Lu D P, Woods G L, Reed E, Zhou G Z, Zhang L B, Waldman R H. Demonstration of the anti-viral activity of garlic extract against human cytomegalovirus in vitro. Chin Med J (Engl) 1993 February; 106(2):93-6.
¹⁴⁰³ Weber N D, Andersen D O, North J A, Murray B K, Lawson L D, Hughes B G. In vitro virucidal effects of Allium sativum (garlic) extract and compounds. Planta Med. 1992 October; 58(5):417-23.
¹⁴⁰⁴ Marchant A, Goetghebuer T, Ota M O, Wolfe I, Ceesay S J, De Groote D, Corrah T, Bennett S, Wheeler J, Huygen K, Aaby P. McAdam K P, Newport M J. Newborns develop a Th1-type immune response to Mycobacterium bovis bacillus Calmette-Guerin vaccination. J. Immunol. 1999 Aug. 15; 163(4):2249-55.
¹⁴⁰⁵ Starr S E, Visintine A M, Tomeh M O, Nahmias A J. Effects of immunostimulants on resistance of newborn mice to herpes simplex type 2 infection. Proc Soc Exp Biol Med. 1976 May; 152(i):57-60.
¹⁴⁰⁶ Aaby P, Shaheen S O, Heyes C B, Goudiaby A, Hall A J, Shiell A W, Jensen H, Marchant A. Early BCG vaccination and reduction in atopy in Guinea-Bissau. Clin Exp Allergy 2000 May; 30(5):644-50.
¹⁴⁰⁷ von Hertzen L C. Puzzling associations between childhood infections and the later occurrence of asthma and atopy. Ann Med 2000 September; 32(6):397-400.
¹⁴⁰⁸ von Mutius E, Pearce N, Beasley R. Cheng S, von Ebrenstein O, Bjorksten B, Weiland S. International patterns of tuberculosis and the prevalence of symptoms of asthma, rhinitis, and eczema. Thorax 2000 June; 55(6):449-53.
¹⁴⁰⁹ von Hertzen L, Klaukka T, Mattila H, Haahtela T. Mycobacterium tuberculosis infection and the subsequent development of asthma and allergic conditions. J Allergy Clin Immunol. 1999 December; 104(6):1211-4.
¹⁴¹⁰ Scanga C B, Le Gros G. Development of an asthma vaccine: research into BCG. Drugs. 2000 June; 59(6):1217-21.
¹⁴¹¹ Shehadeh N, Etzioni A, Cahana A, Teninboum G, Gorodetsky B, Barzilai D, Kamieli E. Repeated BCG vaccination is more effective than a single dose in preventing diabetes in non-obese diabetic (NOD) mice. Isr J Med Sci. 1997 November; 33(11):711-5.
¹⁴¹² Qin H Y, Singh B. BCG vaccination prevents insulin-dependent diabetes mellitus (IDDM) in NOD mice after disease acceleration with cyclophosphamide. J Autoimmun. 1997 June; 10(3):271-8.
¹⁴¹³ Harada M, Kishimoto Y, Makino S. Prevention of overt diabetes and insulitis in NOD mice by a single BCG vaccination. Diabetes Res Clin Pract. 1990 January; 8(2):85-9.
¹⁴¹⁴ Hiltunen M, Lonnrot M, Hyoty H. Immunisation and type I diabetes mellitus: is there a link? Drug Saf. 1999 March; 20(3):207-12.
¹⁴¹⁵ Martins T C, Aguas A P. Mechanisms of Mycobacterium avium-induced resistance against insulin-dependent diabetes mellitus (IDDM) in non-obese diabetic (NOD) mice: role of Fas and Th1 cells. Clin Exp Immunol 1999 February; 115(2):248-54.
¹⁴¹⁶ Bras A, Aguas A P. Diabetes-prone NOD mice are resistant to Mycobacterium avium and the infection prevents autoimmune disease. Immunology. 1996 September; 89(1):20-5.
¹⁴¹⁷ Pabst H F, Spady D W, Pilarski L M, Carson M M, Beeler J A, Krezolek M P. Differential modulation of the immune response by breast- or formula-feeding of infants. Acta Paediatr. 1997 December; 86(12):1291-7.
¹⁴¹⁸ Pabst H F. Immunomodulation by breast-feeding. Pediatr Infect Dis J. 1997 October; 16(10):991-5.
¹⁴¹⁹ Hawkes J S, Neumann M A, Gibson R A. The effect of breast-feeding on lymphocyte subpopulations in healthy term infants at 6 months of age. Pediatr Res 1999 May; 45(5 Pt 1):648-51.
¹⁴²⁰ Pettitt D J, Forman M R, Hanson R L, Knowler W C, Bennett P H. Breastfeeding and incidence of non-insulin-dependent diabetes mellitus in Pima Indians. Lancet. 1997 Jul. 19; 350(9072):166-8.
¹⁴²¹ Virtanen S M, Rasanen L, Aro A, Ylonen K, Lounamaa R, Tuomilehto J, Akerblom H K. Feeding in infancy and the risk of type 1 diabetes mellitus in Finnish children. The ‘Childhood Diabetes in Finland’ Study Group. Diabet Med. 1992 November; 9(9):815-9.
¹⁴²² Virtanen S M, Rasanen L, Aro A, Lindstrom J, Sippola H, Lounamaa R, Toivanen L, Tuomilehto J, Akerblom H K. Infant feeding in Finnish children less than 7 yr of age with newly diagnosed IDDM. Childhood Diabetes in Finland Study Group. Diabetes Care 1991 May; 14(5):415-7.
¹⁴²³Borch-Johnsen K, Joner C, Mandrup-Poulsen T, Christy M, Zachau-Christiansen B, Kastrup K, Nerup J. Relation between breast-feeding and incidence rates of insulin-dependent diabetes mellitus. A hypothesis. Lancet. 1984 Nov. 10; 2(8411):1083-6.
¹⁴²⁴ von Kries R, Koletzko B, Sauerwaid T, von Mutius E. Does breast-feeding protect against childhood obesity? Adv Exp Med Biol. 2000; 478:29-39.
¹⁴²⁵ von Kries R, Koletzko B, Sauerwald T, von Mutius E, Barnert D, Grunert V, von Voss H. Breast-feeding and obesity: cross sectional study. BMJ. 1999 Jul. 17; 319(7203):147-50.
¹⁴²⁶ Bergmann K E, Bergmann R L, Von Kries R, Bohm O, Richter R, Dudenhausen J W, Wahn U. Early determinants of childhood overweight and adiposity in a birth cohort study: role of breast-feeding. Int J Obes Relat Metab Disord. 2003 February; 27(2):162-72.
¹⁴²⁷ Armstrong J, Reilly J J; Child Health Information Team. Breastfeeding and lowering the risk of childhood obesity. Lancet. 2002 Jun. 8; 359(9322):2003-4.
¹⁴²⁸ Toschke A M, Vignerova J, Lhotska L, Osancova K, Koletzko B, Von Kries R. Overweight and obesity in 6- to 14-year-old Czech children in 1991: protective effect of breast-feeding. J Pediatr. 2002 December; 141(6):764-9.
¹⁴²⁹ Liese A D, Hirsch T, von Mutius E, Keil U, Leupold W, Weiland S K. Inverse association of overweight and breast feeding in 9 to 10-y-old children in Germany. Int J Obes Relat Metab Disord. 2001 November; 25(11):1644-50.
¹⁴³⁰ Hediger M L, Overpeck M D, Kuczmarski R J, Ruan W J. Association between infant breastfeeding and overweight in young children. JAMA. 2001 May 16; 285(19):2453-60.
¹⁴³¹ Gillman M W, Rifas-Shiman S L, Camargo C A Jr, Berkey C S, Frazier A L, Rockett H R, Field A E, Colditz G A. Risk of overweight among adolescents who were breastfed as infants. JAMA. 2001 May 16; 285(19):2461-7.
¹⁴³² Gillman M W. Breast-feeding and obesity. J Pediatr. 2002 December; 141(6):749-57.
¹⁴³³ Dietz W H. Breastfeeding may help prevent childhood overweight. JAMA 2001 May 16; 285(19):2506-7.

1. A method for evaluating the ability of a compound to affect expression of a gene natural to a cell, the method comprising the steps of:
a. selecting a transcription complex natural to the cell, wherein the transcription complex is limiting;
b. selecting a polynucleotide foreign to the cell, wherein the foreign polynucleotide can bind the transcription complex;
c. selecting a compound of interest;
d. combining the compound with a system, wherein the system includes a known copy number of the foreign polynucleotide;
e. assaying the copy number of the foreign polynucleotide in the system after the combination; and
f. identifying whether the compound can modify the copy number.

2. The method of claim 1, wherein the cellular transcription complex includes a protein selected from the group consisting of p300 and cbp.

3. The method of claim 1, wherein the cellular transcription complex includes a GABP trans-acting regulatory protein.

4. The method of claim 1, wherein the foreign polynucleotide is a viral polynucleotide.

5. A method for evaluating an effectiveness of a compound for use in modulating progression of a disease, the method comprising the steps of:
a. selecting a transcription complex natural to a cell, wherein the transcription complex is limiting;
b. selecting a polynucleotide foreign to the cell, wherein the polynucleotide can bind the transcription complex;
c. selecting a compound of interest;
d. combining the compound with a system, wherein the system includes a known copy number of the foreign polynucleotide;
e. assaying the copy number of the foreign polynucleotide in the system after the combination; and
f. identifying whether the compound can modify the copy number.

6. The method of claim 5, wherein the cellular transcription complex includes a protein selected from the group consisting of p300 and cbp.

7. The method of claim 5 wherein the cellular transcription complex includes a GABP trans-acting regulatory protein

8. The method of claim 5, wherein the foreign polynucleotide is a viral polynucleotide.

9. The method of claim 5, wherein said chronic disease is selected from the group consisting of atherosclerosis, cancer, obesity, osteoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases.

10. A method for evaluating the ability of a compound to affect expression of a gene natural to a cell, the method comprising the steps of:
a. selecting a transcription complex natural to the cell, wherein the transcription complex is limiting;
b. selecting a polynucleotide foreign to the cell, wherein the foreign polynucleotide can bind the transcription complex;
c. selecting a compound of interest;
d. combining the compound with a system, wherein the system includes the transcription complex and the foreign polynucleotide;
e. assaying the complex between the transcription complex and the foreign polynucleotide in the system after the combination; and
f. identifying whether the compound can modify the complex between the transcription complex and the foreign polynucleotide.

11. The method of claim 10, wherein the cellular transcription complex includes a protein selected from the group consisting of p300 and cbp.

12. The method of claim 10, wherein the cellular transcription complex includes a GABP trans-acting regulatory protein.

13. The method of claim 10, wherein the foreign polynucleotide is a viral polynucleotide.

14. The method of claim 10, wherein the compound can modify the structure or the concentration of the complex between the transcription complex and the foreign polynucleotide.

15. A method for evaluating an effectiveness of a compound for use in modulating progression of a disease, the method comprising the steps of:
a. selecting a transcription complex natural to a cell, wherein the transcription complex is limiting;
b. selecting a polynucleotide foreign to the cell, wherein the foreign polynucleotide can bind the transcription complex;
c. selecting a compound of interest;
d. combining the compound with a system, wherein the system includes the transcription complex;
e. assaying the transcription complex in the system after the combination; and
f. identifying whether the compound can modify the transcription complex.

16. The method of claim 15, wherein the cellular transcription complex includes a protein selected from the group consisting of p300 and cbp.

17. The method of claim 15, wherein the cellular transcription complex includes a GABP trans-acting regulatory protein

18. The method of claim 15, wherein the foreign polynucleotide is a viral polynucleotide.

19. The method of claim 15, wherein the compound can modify the structure or concentration of the transcription complex.

20. The method of claim 15, wherein said chronic disease is selected from the group consisting of atherosclerosis, cancer, obesity, osteoarthritis, type II diabetes, type I diabetes, multiple sclerosis, asthma, lupus, thyroiditis, inflammatory bowel disease, rheumatoid arthritis, psoriasis, atopic dermatitis, graft versus host disease, and other autoimmune diseases.
